(19) United States
(12) Patent Application Publication
(10) Pub. No.: US 2025/0197811 A1
(43) Pub. Date: Jun. 19, 2025

(54) COMPOSITIONS AND METHODS FOR GENERATING CELLS WITH REDUCED IMMUNOGENICITY

(71) Applicant: Celyntra Therapeutics SA, Mont-Saint-Guibert (BE)

(72) Inventors: Tanya Warnecke, Boulder, CO (US); Roland Baumgartner, Boulder, CO (US); John Schiel, Boulder, CO (US); Nicholas Eion Timmins, Boulder, CO (US)

(73) Assignee: Celyntra Therapeutics SA, Mont-Saint-Guibert (BE)

(21) Appl. No.: 18/847,689

(22) PCT Filed: Mar. 22, 2023
(86) PCT No.: PCT/US2023/015978

§ 371 Date: Sep. 17, 2024

Related U.S. Application Data
(60) Provisional application No. 63/322,634, filed on Mar. 22, 2022.

(51) Int. Cl. C12N 5/074; C12N 5/0783; C12N 9/22; C12N 15/62; C12N 15/85

(52) U.S. Cl. CPC C12N 5/0696; C12N 5/0636; C12N 9/22; C12N 15/62; C12N 15/85; C12N 2310/20; C12N 2510/00; C12N 2810/00

(57) ABSTRACT

CRISPR-Cas systems have been engineered for various purposes, such as genomic DNA cleavage, base editing, epigenome editing, and genomic imaging. Although significant developments have been made, there still remains a need for new and useful CRISPR-Cas systems as powerful precise genome targeting tools. The invention disclosed herein comprises CRISPR-Cas based methods for high integration and expression efficiency of transgenes together with high post-transfection cell viability in eukaryotic cells.

### REFERENCE TO RELATED APPLICATIONS

This application claims the benefit of and priority to U.S. Provisional Patent Application No. 63/322,634, filed Mar. 22, 2022, the disclosure of which is hereby incorporated by reference in its entirety for all purposes.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Current cell therapy products, e.g., CAR T cells, recover cells from the prospective patient wherein those cells are then modified, optionally expanded, and then used for one or more treatments. The overall process is time consuming, which can negatively impact the success the treatment outcome, and expensive. As a result, there is a strong need to develop on-demand, reasonably priced, allogeneic cell therapy products that demonstrate reduced immunogenicity, e.g., reduced Graft versus Host and/or Host versus Graft response.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows a schematic representation showing the structure of an exemplary single guide Type V-A CRISPR system. FIG. 1B is a schematic representation showing the structure of an exemplary dual guide Type V-A CRISPR system.

FIGS. 2A-C show a series of schematic representation showing incorporation of a protecting group (e.g., a protective nucleotide sequence or a chemical modification) (FIG. 2A), a donor template-recruiting sequence (FIG. 2B), and an editing enhancer (FIG. 2C) into a Type V-A CRISPR-Cas system. These additional elements are shown in the context of a dual guide Type V-A CRISPR system, but it is understood that they can also be present in other CRISPR systems, including a single guide Type V-A CRISPR system, a single guide Type II CRISPR system, or a dual guide Type II CRISPR system.

FIG. 3 shows percent of treated cell populations (A) triple knock-out of TCR, HLA-I, and HLA-II, or (B) triple KO TCR, HLA-I, HLA-II, and insertion of a CAR after treatment as measured by flow cytometry; FL=full length, ldsPLA074=linear DNA used to insert CAR.

FIG. 4 shows reduced HLA-I, HLA-II, and/or TCR surface expression (y-axis) in cells transfected with RNPs comprising a nucleic acid-guided nuclease complexed with various gCD3D gNAs.

FIG. 5 shows reduced HLA-I, HLA-II, and/or TCR surface expression (y-axis) in cells treated with various RNPs comprising a nucleic acid-guided nuclease complexed with CD247, CD3G, or TRAC gNAs.

FIG. 6A shows reduced TCR surface expression (y-axis) in cells transfected with RNPs comprising a nucleic acid-guided nuclease complexed with TRBC gNAs.

FIG. 6B shows simultaneous TRBC KO and CAAR KI (CAAR expression, y-axis) in cells transfected with RNPs comprising a nucleic acid-guided nuclease complexed with TRBC gNAs and repair template.

FIG. 7 shows reduced TRC surface expression (7A, y-axis) in cells transfected with RNPs comprising a nucleic acid-guided nuclease complexed with CD3E gNAs; and simultaneous CD3E KO and CAR KI (CAR expression, y-axis, 7B) in cells transfected with RNPs comprising a nucleic acid-guided nuclease complexed with TRBC gNAs and repair template.

### DETAILED DESCRIPTION

### Outline

I. Cells with reduced immunogenicity
A. Compositions comprising cells
   1. Cells comprising genomic modifications
   2. Cell populations comprising genomic modifications
   3. Guide nucleic acids and nucleic acid-guided nuclease complexes for generating genomic modifications
B. Methods for reducing immunogenicity of cells
II. Engineered non-naturally occurring dual guide CRISPR-cas systems
A. Cas proteins
B. Guide nucleic acids
C. gNA modifications
III. Composition and methods for targeting, editing, and/or modifying genomic DNA
A. Ribonucleoprotein (RNP) delivery and “cas RNA” delivery
B. CRISPR expression systems
C. Donor templates
D. Efficiency and specificity
E. Multiplex
F. Genomic safe harbors
IV. Pharmaceutical compositions
V. Therapeutic uses
A. Gene therapies
VI. Kits
VII. Embodiments
VIII. Examples
IX. Equivalents

### I. CELLS WITH REDUCED IMMUNOGENICITY

The immune system recognizes specific antigen patterns on the cell surface, e.g., in humans, human leukocyte antigen (HLA) proteins. These patterns of protein antigens are genetically determined and vary between individuals, where an individual's immune system recognizes its own specific antigen pattern as “self” and those antigen patterns that differ as “non-self” or “foreign”. Typically, foreign cells, e.g., allogeneic cells (cells from a genetically dissimilar individual), and/or those demonstrating HLA patterns different than expected, elicit one or more immune responses in the host. In the context of cell therapy applications, this immune response, termed “Host versus Graft” (HvG), can hinder and/or reduce the efficacy of the one or more therapeutic agents as the body recognizes the therapeutic agent as foreign and targets the therapeutic agent for removal.

Further, engineered cells, e.g., modified cells, used in cell therapy can recognize the antigen pattern of host cells as foreign and elicit an immune response. This immune response, as herein termed “Graft versus Host” (GvH), can result in the therapy demonstrating a negative and/or harmful effect on the recipient.

Provided herein are compositions, methods, and/or kits for generating a cell that demonstrates reduced immunogenicity. In certain embodiments, provided herein are cells comprising one or more modifications that result in reduced HvG, GvH, and/or both. In certain embodiments, the cell comprises eukaryotic cells. In certain embodiments, the cell comprises human cells. In certain embodiments, the cell comprises a human immune cell such as a neutrophil, cosinophil, basophil, mast cell, monocyte, macrophage, dendritic cell, natural killer cell, a lymphocyte, or a combination thereof, for example a T cell. In preferred embodiments, the cell comprises a T cell. In certain embodiments, the cell comprises an engineered immune cell, for example a chimeric antigen receptor (CAR)-T cell comprising one or more CAR polypeptides or portions thereof and/or a dual CAR. In certain embodiments, the cell comprises a human stem cell such as a human pluripotent, multipotent stem cell, embryonic stem cell, induced pluripotent stem cell, hematopoietic stem cell, a CD34+ cell, or a combination thereof. In preferred embodiments, the human stem cell comprises hematopoietic stem cells, CD34+ stem cells, and/or induced pluripotent stem cells (iPSC). In certain embodiments, the cell comprises an allogeneic cell. As used herein, the term “allogeneic” includes cells from the same species that are genetically dissimilar and hence immunologically incompatible with the host.

In certain embodiments, provided herein are compositions, methods, and/or kits comprising dual CARs, e.g., a CAR fusion protein or two separate CARs. As used herein, the term “dual CAR” includes a polypeptide comprising a first CAR or portion thereof and a second CAR or portion thereof, either separate, or connected via one or more polypeptide linkers. In certain embodiments, the second CAR or portion thereof targets the same antigen as the first CAR or portion thereof. In certain embodiments, the second CAR or portion thereof targets a different antigen than the first CAR or portion thereof. Additionally disclosed herein are polypeptides comprising any number of CARs or portions thereof, separate or connected via one or more polypeptide linkers. In certain embodiments, a cell can comprise at least 1, 2, 3, 4, 5, 6, 7, 8, 9, 10, 11, 12, 13, or 14 and/or no more than 15, 14, 13, 12, 11, 10, 9, 8, 7, 6, 5, 4, 3, or 2 CARs or portions thereof, for example 1-15, preferably 1-10, more preferably, 2-10, even more preferably 2-7, yet more preferably 2-5 CARs or portions thereof, separately or connected via one or more polypeptide linkers. The polypeptide linker can comprise any suitable linker comprising natural or unnaturally occurring amino acids.

In certain embodiments, a cell can be engineered to comprise one or more genomic modifications. In certain embodiments, the cell can be engineered to comprise one or more genomic modifications that reduce the immunogenicity of the cells, e.g., the modified cell results in little to no immune response in vitro and/or in vivo. In certain embodiments, an allogeneic cell with respect to a host (recipient, patient, or suitable alternative) can be engineered to comprise one or more genomic modifications that reduce the immunogenicity of the one or more allogeneic cells in the host. In certain embodiments, the cell can be engineered to elicit no more than 90, 80, 70, 60, 50, 40, 30, 25, 20, 15, 10, 9, 8, 7, 6, 5, 4, 3, 2, or 1% of the immune response as compared to an un-engineered equivalent. In certain embodiments, the cell can be engineered to elicit no immune response in a host. The immune response can be measured using any suitable technique, for example, flow cytometry or an ELISA.

In certain embodiments, the cell comprises (1) one or more genomic modifications that partially or completely inactivates one or more genes that codes for a subunit of an HLA-1 protein, (2) one or more genomic modifications that partially or completely inactivates one or more genes coding for a subunit of an HLA-2 protein or a transcription factor regulating the expression of one or more subunits of an HLA-2 protein, and/or (3) one or more genomic modifications that partially or completely inactivates one or more genes that codes for a subunit of a TCR protein. In a preferred embodiment, the cell comprises all three genomic modifications. In certain embodiments, the one or more genomic modifications completely inactivates the one or more genes. In certain embodiments, the one or more genomic modifications at least partially or completely eliminates surface expression of active (immunogenic) proteins. In certain embodiments, the one or more genomic modifications completely eliminates surface expression of active (immunogenic) proteins. In certain embodiments, the cell comprising the one or more genomic modifications can further comprise one or more additional modifications including, but not limited to, introduction of one or more heterologous genes, e.g., transgenes. The one or more transgenes can be introduced into any suitable location in the genome. In certain embodiments, the one or more transgenes are introduced into a safe harbor site (SHS), e.g., a safe harbor, as discussed in the Genomic safe harbors section below. In certain embodiments, the one or more transgenes are introduced into one or more of the sites comprising a genomic modification (1) through (3), for example, a CAR transgene can be introduced into one or more genes coding for a subunit of a TCR protein, e.g., a TRAC gene, and/or a B2M-HLA-E and/or a B2M HLA-G fusion protein can be introduced into one or more genes coding for a subunit of an HLA-1 protein, e.g., a B2M gene.

In certain embodiments, provided herein are compositions comprising one or more populations of cells having genetic modifications as described herein. In certain embodiments, the composition comprises a single cell population, wherein each of the cells comprises the same set of genomic modifications (1) through (3). In certain embodiments, provided herein are compositions comprising a plurality of cell populations, wherein each cell population comprises a different set of genomic modifications. In general, at least one cell population comprises cells that comprise all of (1) one or more genomic modifications that partially or completely inactivates one or more genes that codes for a subunit of an HLA-1 protein, (2) one or more genomic modifications that partially or completely inactivates one or more genes coding for a subunit of an HLA-2 protein or a transcription factor regulating the expression of one or more subunits of an HLA-2 protein, and (3) one or more genomic modifications that partially or completely inactivates one or more genes that codes for a subunit of a TCR protein, in addition to one or more additional cell populations that do not comprise all three genetic modifications. In certain embodiments, the one or more additional cell populations comprise cells comprising (1) one or more genomic modifications that partially or completely inactivates one or more genes that codes for a subunit of an HLA-1 protein, (2) one or more genomic modifications that partially or completely inactivates one or more genes coding for a subunit of an HLA-2 protein or a transcription factor regulating the expression of one or more subunits of an HLA-2 protein, and/or (3) one or more genomic modifications that partially or completely inactivates one or more genes that codes for a subunit of a TCR protein, but not all of (1)-(3). In a preferred embodiment, the subunit of an HLA-1 protein comprises B2M. In a preferred embodiment, the transcription factor regulating the expression of one or more subunits of an HLA-2 protein comprises CIITA. In certain embodiments, the subunit of a TCR protein is an alpha subunit or a beta subunit. In a preferred embodiment, the gene that codes for a subunit of a TCR protein is a TRAC gene. In certain embodiments, the subunit of a TCR protein is a TRAC, TRBC, CD3E, CD3D, CD3G, or CD3Z protein. In a more preferred embodiment, at least one cell population comprises cells that comprise all of (1) one or more genomic modifications that partially or completely inactivates a B2M gene, (2) one or more genomic modifications that partially or completely inactivates a CIITA gene, and (3) one or more genomic modifications that partially or completely inactivates a TRC subunit gene, e.g., a TRAC gene, in addition to one or more additional cell populations one or more, but not all three, genomic modifications. In certain embodiments, the one or more genomic modifications at least partially or completely eliminates surface expression of active (immunogenic) proteins. In certain embodiments, the one or more genomic modifications completely eliminates surface expression of active (immunogenic) proteins. In certain embodiments, the one or more cells comprising the one or more genomic modifications can further comprise one or more additional modifications including, but not limited to, introduction of one or more heterologous genes, e.g., transgenes. The one or more transgenes can be introduced into any suitable location in the genome. In certain embodiments, the one or more transgenes are introduced into a safe harbor site (SHS), e.g., a safe harbor, as discussed in the Genomic safe harbors section below. In certain embodiments, the one or more transgenes are introduced into one or more of the sites comprising a genomic modification (1) through (3), for example, a CAR transgene can be introduced into one or more genes coding for a subunit of a TCR protein, e.g., a TRAC gene, and/or a B2M-HLA-E and/or a B2M HLA-G fusion protein can be introduced into one or more genes coding for a subunit of an HLA-1 protein, e.g., a B2M gene. In certain embodiments, the plurality of cell populations comprise at least 1, 2, 3, 4, 5, 6, 7, 8, 9, 10, 11, 12, 13, 14, 15, 16, 17, 18, 19, 20, 25, 30, 35, 40, or 45 and/or no more than 2, 3, 4, 5, 6, 7, 8, 9, 10, 11, 12, 13, 14, 15, 16, 17, 18, 19, 20, 25, 30, 35, 40, 45, or 50 cell populations, for example 1-50 cell populations.

Cells can be engineered using any suitable composition and method. In certain embodiments, a cell can be engineered by delivering to the cell a composition comprising a site-specific nuclease and/or one or more polynucleotides encoding for the site-specific nuclease. The site-specific nuclease can be any suitable nuclease, such as a homing endonuclease, a TALEN, a meganuclease, an argonaut, and/or a CRISPR/Cas nuclease, i.e., a nucleic acid guided nuclease. In preferred embodiments, the site-specific nuclease comprises a nucleic acid-guided nuclease. The site-specific nuclease can hydrolyze the backbone, i.e., generate one or more cuts or strand breaks, in the DNA duplex, at or near the nuclease's recognition site, i.e., the target site. The one or more strand breaks in at least one strand of the DNA can be repaired via any suitable innate cell repair mechanism, such as non-homologous recombination (NHEJ) and/or homology directed repair (HDR). In certain embodiments, repair one or more strand breaks in at least one strand of the DNA by NHEJ results in one or more genomic modifications, such as insertions and/or deletions (INDELS). In certain embodiments, one or more portions of heterologous DNA, e.g., donor template, can be introduced into the cells and at least a portion of the heterologous DNA can be inserted by the cell at or near the one or more strand breaks in the DNA by HDR.

In certain embodiments, the site-specific nuclease comprises a nucleic acid-guided nuclease, e.g., a CRISPR/Cas nuclease. In certain embodiments, nucleic acid-guided nuclease comprises one or more engineered, non-naturally occurring components. In certain embodiments, the nucleic acid-guided nuclease comprises a Class 1 or Class 2 Cas nuclease, such as a Type V-A, V-B, V-C, V-D, or V-E. In certain embodiments, the nucleic acid-guided nuclease comprises an amino acid sequence at least 80%, at least 85%, at least 90%, at least 95%, at least 99%, or 100% identical to an amino acid sequence of a MAD, ART, or ABW nuclease, such as a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, MAD20, ARTI, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, and/or ART35 nuclease. In preferred embodiments, the nucleic acid-guided nuclease comprises a MAD2, MAD7, ART11, ART11*, or ART2 nuclease. In more preferred embodiments, the nucleic acid-guided nuclease comprises an amino acid sequence at least 80%, at least 85%, at least 90%, at least 95%, at least 99%, or 100% identical, to the amino acid sequence of MAD2, MAD7, ART2, ART11, or ART11*. In even more preferred embodiments, the nucleic acid-guided nuclease comprises an amino acid sequence that is at least 80%, at least 85%, at least 90%, at least 95%, 99%, or 100% identical, to the amino acid sequence of SEQ ID NO: 37. In certain embodiments, the nucleic acid-guided nuclease comprises one or more nuclear localization signals (NLS), for example 1, 4, or 5 nuclear localization signals, such as 1-5 NLS at the carboxy terminus, 1-5 NLS at the amino terminus, or a combination thereof. In certain embodiments, provided herein the nucleic-acid guided nuclease comprises one N-terminal NLS and 3 C-terminal NLS. In certain embodiments, the one or more NLS comprises SEQ ID NOS: 40, 51, and 56. Additional nucleases and modifications thereof may be found in the Cas proteins section below.

In certain embodiments, the nucleic acid-guided nuclease further comprises a guide nucleic acid. In certain embodiments, the guide nucleic acid comprises a targeter nucleic acid and a modulator nucleic acid. In certain embodiments, the targeter nucleic acid comprises a targeter nucleic acid comprising a targeter stem sequence and a spacer sequence. In certain embodiments, the modulator nucleic acid comprises a modulator stem sequence complementary to the targeter stem sequence, and, optionally, a 5′ sequence. In certain embodiments, the guide nucleic acid comprises a single polynucleotide. In certain embodiments, the guide nucleic acid comprises an engineered, non-naturally occurring guide nucleic acid. In certain embodiments, the guide nucleic acid comprises a dual guide nucleic acid (as described in the Guide nucleic acids section below), wherein the targeter nucleic acid and the modulator nucleic acid are separate polynucleotides. In certain embodiments wherein the guide nucleic acid is a dual guide nucleic acid, the stem of the targeter nucleic acid and the stem of the modulator nucleic acid hybridize. In certain embodiments, the dual guide nucleic acid is capable of binding to and activating a nucleic acid-guided nuclease, that, in a naturally occurring system, is activated by a single cRNA in the absence of a tracrRNA.

In certain embodiments, the guide nucleic acid comprises one or more chemical modifications to one or more nucleotides and/or internucleotide linkages at or near the 5′ end, the 3′ end, and/or both as described in the gNA modifications section below. In certain embodiments, the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, or a combination thereof.

In certain embodiments, provided herein are guide nucleic acids comprising a spacer sequence at least partially complementary to a site (1) within one or more genes that codes for a subunit of an HLA-1 protein, (2) within one or more genes coding for a subunit of an HLA-2 protein or a transcription factor regulating the expression of one or more subunits of an HLA-2 protein, and/or (3) within one or more genes that codes for a subunit of a TCR protein.

In certain embodiments, the one or more guide nucleic acids can be complexed with one or more nucleases, e.g., a nucleic acid-guided nuclease complex. In certain embodiments, provided herein are nucleic acid-guided nuclease complexes comprising a nucleic acid-guided nuclease and a compatible guide nucleic acid comprising a spacer sequence at least partially complementary to a site (1) within one or more genes that codes for a subunit of an HLA-1 protein, (2) within one or more genes coding for a subunit of an HLA-2 protein or a transcription factor regulating the expression of one or more subunits of an HLA-2 protein, and/or (3) within one or more genes that codes for a subunit of a TCR protein. In certain embodiments, the one or more guide nucleic acids, one or more nucleic acid guided nucleases, and/or the one or more nucleic acid-guided nucleases may further comprise a one or more additives that stabilize the nucleic acid-guided nuclease complex.

Such cells and/or populations of cells with lowered immunogenicity can be used for a variety of purposes, one such purpose can be a CAR T cell.

### A. Compositions Comprising Cells

### 1. Cells Comprising Genomic Modifications

In certain embodiments, provided herein are compositions comprising cells comprising one or more genomic modifications that reduce or eliminate an immune response to the cells in an allogeneic host. The one or more genomic modifications can alter the surface expression of one or more antigens affecting the immunogenicity of the one or more modified cells, e.g., by partially or completely inactivating a gene that codes for the antigen, or part of the antigen. In certain embodiments, the cell comprising one or more genomic modifications are generated from an initial cell not comprising genomic modifications affecting immunogenicity, e.g., a primary cell or a stem cell. In certain embodiments, an initial, unmodified, cell is modified so that all desired genetic modifications are introduced into the cell. In other embodiments, a sequential process is used, e.g., a cell is modified so that part of the desired modifications is introduced, then one or more of its progeny is further modified; this sequential approach can be two steps, three steps, four steps, or more. That is, a cell comprising one or more genomic modifications is, optionally expanded and used as a starting point for introduction of one or more additional genomic modifications. In certain embodiments wherein the cell comprises a stem cell, the stem cell can be differentiated before and/or after introduction of one or more genomic modifications. Additional methods are described in the Methods for reducing immunogenicity of cells section below. In certain embodiments, a composition comprising the one or more cells comprising one or more genomic modifications further comprises a pharmaceutically acceptable excipient.

a. Cells Comprising Modifications that Result in Partial or Complete Inactivation of a Gene Coding for a Subunit of HLA-1

In certain embodiments, provided herein are compositions comprising a cell comprising a first genomic modification in a gene that codes for a subunit of an HLA-1 protein. In certain embodiments, the first genomic modification partially or completely inactivates the gene that codes for a subunit of an HLA-1 protein. In certain embodiments, the first genomic modification completely inactivates the gene that codes for a subunit of an HLA-1 protein. In certain embodiments, the first genomic modification reduces or eliminates surface expression of active (immunogenic) HLA-1 proteins. In certain embodiments, the first genomic modification completely eliminates surface expression of active (immunogenic) HLA-1 proteins. In certain embodiments, the gene that codes for a subunit of an HLA-1 protein comprises a B2M gene. In certain embodiments, the first genomic modification comprises a substitution, an insertion, a deletion, a nonsense mutation, a truncation, or a combination thereof. In certain embodiments, the first genomic modification comprises insertion of heterologous DNA, e.g., a transgene, for example a transgene comprising a polynucleotide coding for a B2M-fusion protein, such as a B2M-HLA fusion protein, e.g., a B2M-HLA-E fusion protein or a B2M-HLA-G fusion protein.

In certain embodiments, the cell is a human cell, such as human stem cell or human immune cell, such as an immune cell comprising a neutrophil, cosinophil, basophil, mast cell, monocyte, macrophage, dendritic cell, natural killer cell, or a lymphocyte. In a preferred embodiment, the human immune cell is a T cell. In certain embodiments, the T cell comprises a chimeric antigen receptor (CAR) T cell. In certain embodiments, the CAR T cell expresses a plurality of different CARs, e.g., two different CARs (dual CAR T cell). In certain embodiments, the human cell is a human stem cell comprising a human pluripotent stem cell, multipotent stem cell, embryonic stem cell, induced pluripotent stem cell, hematopoietic stem cell, or a CD34+ cell. In a preferred embodiment, the cell is a hematopoietic stem cell. In a more preferred embodiment, the cell is a CD34+ stem cell. In an even more preferred embodiment, the cell is an induced pluripotent stem cell (iPSC).

In certain embodiments, the cell further comprises one or more nucleic acid-guided nucleases, one or more guide nucleic acids, and/or one or more polynucleotides encoding the one or more nucleic acid-guided nucleases and/or guide nucleic acids. In a preferred embodiment, the cell comprises a nucleic acid-guided nuclease complexed with a gRNA. In certain embodiments, one or more of the nucleic acid-guided nucleases (see Cas nucleases section below) are complexed with one or more of the guide nucleic acids (see Guide nucleic acids section below). In certain embodiments, the nuclease comprises a Type V nuclease. In a preferred embodiment, the nuclease comprises a Type V-A nuclease. In an even more preferred embodiment, the nuclease comprises MAD7, e.g., MAD7 comprising one or more nuclear localization signals (NLS), for example one to four NLS, preferably four NLS, more preferably one N-terminal NLS and three C-terminal NLS. In certain embodiments, the cell further comprises a donor template, such as a donor template described herein, e.g., a donor template comprising a polynucleotide coding for one or more CARs or portions thereof.

In certain embodiments, the cell further comprises a second genomic modification comprising a first transgene inserted into the genome. The first transgene can be inserted into any suitable location in the genome of the cell. In certain embodiments, the first transgene is inserted into a safe harbor site. The safe harbor site can be any suitable safe harbor site (see Genomic safe harbors section below). In certain embodiments, the safe harbor site comprises an AAVS1 or Rosa 26 locus. In certain embodiments the safe harbor site comprises any one of SEQ ID NOS: 2020-2043. In preferred embodiments, the first transgene is inserted into the gene that codes for the subunit of an HLA-1 protein, e.g., a B2M gene. In certain embodiments, the first transgene comprises a polynucleotide coding for B2M fusion protein, such as a B2M-HLA-1 subunit fusion protein. In certain embodiments, the HLA-1 subunit comprises HLA-C, HLA-E, or HLA-G, preferably HLA-E or HLA-G. In a preferred embodiment the subunit is HLA-E. In a more preferred embodiment, the subunit is HLA-G. Additionally or alternatively, the cell can comprise a transgene comprising a polynucleotide coding for a CAR or portion thereof. In certain embodiments, the transgene comprises a polynucleotide coding for a dual CAR or portions thereof, e.g., a CAR or portion thereof fusion protein. In certain embodiments, the dual CAR comprises a first CAR or portion thereof and a second CAR or portion thereof, wherein the second CAR or portion thereof is different from the first CAR or portion thereof. In certain embodiments, the CAR or portion thereof comprises a polypeptide that binds at least one of B7H3, BCMA, GPRC5D, CD8, CD8a, CD19, CD20, CD22, CD28, 4-1BB, or CD3zeta. In a preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-124. In a more preferred embodiment, the CAR or portion thereof comprises a polypeptide that binds at least one of B7H3, BCMxA, GPRC5D, CD8, CD8a, CD20, CD22, CD28, 4-1BB, or CD3zeta. In an even more preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

b. Cells Comprising Modifications that Result in Partial or Complete Inactivation of a Gene Coding for a Subunit of HLA-1 and HLA-2

In certain embodiments, provided herein are compositions comprising a cell comprising a first genomic modification in a gene that codes for a subunit of an HLA-1 protein, as described above, and a second genomic modification in a gene that codes for a subunit of an HLA-2 protein or a transcription factor regulating the expression of one or more subunits of an HLA-2 protein. In certain embodiments, the first genomic modification partially or completely inactivates the gene that codes for a subunit of an HLA-1 protein and/or the second genomic modification partially or completely inactivates the gene that codes for a subunit of an HLA-2 protein or transcription factor regulating the expression of one or more subunits of an HLA-2 protein. In certain embodiments, the first genomic modification completely inactivates the gene that codes for a subunit of an HLA-1 protein and/or the second genomic modification partially or completely inactivates the gene that codes for a subunit of an HLA-2 protein or transcription factor regulating the expression of one or more subunits of an HLA-2 protein. In certain embodiments, the first and/or second genomic modification reduces or eliminates surface expression of active (immunogenic) HLA-1 and/or HLA-2 proteins. In certain embodiments, the first and/or second genomic modification completely eliminates surface expression of active (immunogenic) HLA-1 and/or HLA-2 proteins. In certain embodiments, the gene that codes for a subunit of an HLA-1 protein comprises a B2M gene. In certain embodiments, the gene that codes for a transcription factor regulating the expression of one or more subunits of an HLA-2 protein comprises a CIITA gene. In certain embodiments, the first and/or second genomic modification comprises a substitution, an insertion, a deletion, a nonsense mutation, a truncation, or a combination thereof. In certain embodiments, the first genomic modification comprises insertion of heterologous DNA, e.g., a transgene, for example a transgene comprising a polynucleotide coding for a B2M-fusion protein, such as a B2M-HLA fusion protein, e.g., a B2M-HLA-E fusion protein or a B2M-HLA-G fusion protein.

In certain embodiments, the cell is a human cell, such as human stem cell or human immune cell, such as an immune cell comprising a neutrophil, cosinophil, basophil, mast cell, monocyte, macrophage, dendritic cell, natural killer cell, or a lymphocyte. In a preferred embodiment, the human immune cell is a T cell. In certain embodiments, the T cell comprises a chimeric antigen receptor (CAR) T cell. In certain embodiments, the CAR T cell expresses a plurality of different CARs, e.g., two different CARs (dual CAR T cell). In certain embodiments, the human cell is a human stem cell comprising a human pluripotent stem cell, multipotent stem cell, embryonic stem cell, induced pluripotent stem cell, hematopoietic stem cell, or a CD34+ cell. In a preferred embodiment, the cell is a hematopoietic stem cell. In a more preferred embodiment, the cell is a CD34+ stem cell. In an even more preferred embodiment, the cell is an induced pluripotent stem cell (iPSC).

In certain embodiments, the cell further comprises one or more nucleic acid-guided nucleases, one or more guide nucleic acids, and/or one or more polynucleotides encoding the one or more nucleic acid-guided nucleases and/or guide nucleic acids. In a preferred embodiment, the cell comprises a nucleic acid-guided nuclease complexed with a gRNA. In certain embodiments, one or more of the nucleic acid-guided nucleases (see Cas nucleases section below) are complexed with one or more of the guide nucleic acids (see Guide nucleic acids section below). In certain embodiments, the nuclease comprises a Type V nuclease. In a preferred embodiment, the nuclease comprises a Type V-A nuclease. In an even more preferred embodiment, the nuclease comprises MAD7, e.g., MAD7 comprising one or more nuclear localization signals (NLS), for example one to four NLS, preferably four NLS, more preferably one N-terminal NLS and three C-terminal NLS. In certain embodiments, the cell further comprises a donor template, such as a donor template described herein, e.g., a donor template comprising a polynucleotide coding for one or more CARs or portions thereof.

In certain embodiments, the cell further comprises a third genomic modification comprising a first transgene inserted into the genome. The first transgene can be inserted into any suitable location in the genome of the cell. In certain embodiments, the first transgene is inserted into a safe harbor site. The safe harbor site can be any suitable safe harbor site (see Genomic safe harbors section below). In certain embodiments, the safe harbor site comprises an AAVS1 or Rosa 26 locus. In certain embodiments the safe harbor site comprises any one of SEQ ID NOS: 2020-2043. In preferred embodiments, the first transgene is inserted into the gene that codes for the subunit of an HLA-1 protein, e.g., a B2M gene. In certain embodiments, the first transgene comprises a polynucleotide coding for B2M fusion protein, such as a B2M-HLA-1 subunit fusion protein. In certain embodiments, the HLA-1 subunit comprises HLA-C, HLA-E, or HLA-G, preferably HLA-E or HLA-G. In a preferred embodiment the subunit is HLA-E. In a more preferred embodiment, the subunit is HLA-G. Additionally or alternatively, the cell can comprise a transgene comprising a polynucleotide coding for a CAR or portion thereof. In certain embodiments, the transgene comprises a polynucleotide coding for a dual CAR or portions thereof, e.g., a CAR or portion thereof fusion protein. In certain embodiments, the dual CAR comprises a first CAR or portion thereof and a second CAR or portion thereof, wherein the second CAR or portion thereof is different from the first CAR or portion thereof. In certain embodiments, the CAR or portion thereof comprises a polypeptide that binds at least one of B7H3, BCMA, GPRC5D, CD8, CD8a, CD19, CD20, CD22, CD28, 4-1BB, or CD3zeta. In a preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-124. In a more preferred embodiment, the CAR or portion thereof comprises a polypeptide that binds at least one of B7H3, BCMxA, GPRC5D, CD8, CD8a, CD20, CD22, CD28, 4-1BB, or CD3zeta. In an even more preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

c. Cells Comprising Modifications that Result in Partial or Complete Inactivation of a Gene Coding for a Subunit of HLA-1, HLA-2, and TCR

In certain embodiments, provided herein are compositions comprising a cell comprising a first genomic modification in a gene that codes for a subunit of an HLA-1 protein, a second genomic modification in a gene that codes for a subunit of an HLA-2 protein or a transcription factor regulating the expression of one or more subunits of an HLA-2 protein, as described above, and a third genomic modification in a gene that codes for a subunit of a TCR protein. In certain embodiments, the first genomic modification partially or completely inactivates the gene that codes for a subunit of an HLA-1 protein, the second genomic modification partially or completely inactivates the gene that codes for a subunit of an HLA-2 protein or transcription factor regulating the expression of one or more subunits of an HLA-2 protein, and/or the third genomic modification partially or completely inactivates the gene that codes for a subunit of a TCR protein. In certain embodiments, the first genomic modification completely inactivates the gene that codes for a subunit of an HLA-1 protein, the second genomic modification partially or completely inactivates the gene that codes for a subunit of an HLA-2 protein or transcription factor regulating the expression of one or more subunits of an HLA-2 protein, and/or the third genomic modification partially or completely inactivates the gene that codes for a subunit of a TCR protein. In certain embodiments, the first, second, and/or third genomic modification reduces or eliminates surface expression of active (immunogenic) HLA-1, HLA-2 proteins, and/or TCR proteins. In certain embodiments, the first, second, and/or third genomic modifications completely eliminate surface expression of active (immunogenic) HLA-, HLA-2, and/or TCR proteins. In certain embodiments, the gene that codes for a subunit of an HLA-1 protein comprises a B2M gene. In certain embodiments, the gene that codes for a transcription factor regulating the expression of one or more subunits of an HLA-2 protein comprises a CIITA gene. In certain embodiments, the subunit of a TCR protein comprises an alpha or a beta subunit. In certain embodiments, the subunit of a TCR protein is a TRAC, TRBC, CD3E, CD3D, CD3G, or CD3Z protein. In certain embodiments, the subunit of a TCR protein comprises an alpha subunit. In certain embodiment, the gene that codes for a subunit of a TCR protein comprises a TRAC gene. In certain embodiments, the first, second, and/or third genomic modifications comprise a substitution, an insertion, a deletion, a nonsense mutation, a truncation, or a combination thereof. In certain embodiments, the first genomic modification comprises insertion of heterologous DNA, e.g., a transgene, for example a transgene comprising a polynucleotide coding for a B2M-fusion protein, such as a B2M-HLA fusion protein, e.g., a B2M-HLA-E fusion protein or a B2M-HLA-G fusion protein. In certain embodiments, the third genomic modification comprises insertion of heterologous DNA, e.g., a transgene, for example a polynucleotide coding for a CAR protein or a dual CAR protein.

In certain embodiments, the cell is a human cell, such as human stem cell or human immune cell, such as an immune cell comprising a neutrophil, cosinophil, basophil, mast cell, monocyte, macrophage, dendritic cell, natural killer cell, or a lymphocyte. In a preferred embodiment, the human immune cell is a T cell. In certain embodiments, the T cell comprises a chimeric antigen receptor (CAR) T cell. In certain embodiments, the CAR T cell expresses a plurality of different CARs, e.g., two different CARs (dual CAR T cell). In certain embodiments, the human cell is a human stem cell comprising a human pluripotent stem cell, multipotent stem cell, embryonic stem cell, induced pluripotent stem cell, hematopoietic stem cell, or a CD34+ cell. In a preferred embodiment, the cell is a hematopoietic stem cell. In a more preferred embodiment, the cell is a CD34+ stem cell. In an even more preferred embodiment, the cell is an induced pluripotent stem cell (iPSC).

In certain embodiments, the cell further comprises one or more nucleic acid-guided nucleases, one or more guide nucleic acids, and/or one or more polynucleotides encoding the one or more nucleic acid-guided nucleases and/or guide nucleic acids. In a preferred embodiment, the cell comprises a nucleic acid-guided nuclease complexed with a gRNA. In certain embodiments, one or more of the nucleic acid-guided nucleases (see Cas nucleases section below) are complexed with one or more of the guide nucleic acids (see Guide nucleic acids section below). In certain embodiments, the nuclease comprises a Type V nuclease. In a preferred embodiment, the nuclease comprises a Type V-A nuclease. In an even more preferred embodiment, the nuclease comprises MAD7, e.g., MAD7 comprising one or more nuclear localization signals (NLS), for example one to four NLS, preferably four NLS, more preferably one N-terminal NLS and three C-terminal NLS. In certain embodiments, the cell further comprises a donor template, such as a donor template described herein, e.g., a donor template comprising a polynucleotide coding for one or more CARs or portions thereof.

In certain embodiments, the cell further comprises a fourth genomic modification comprising a first transgene inserted into the genome. The first transgene can be inserted into any suitable location in the genome of the cell. In certain embodiments, the first transgene is inserted into a safe harbor site. The safe harbor site can be any suitable safe harbor site (see Genomic safe harbors section below). In certain embodiments, the safe harbor site comprises an AAVS1 or Rosa 26 locus. In certain embodiments the safe harbor site comprises any one of SEQ ID NOs: 2020-2043. In preferred embodiments, the first transgene is inserted into the gene that codes for the subunit of an HLA-1 protein, e.g., a B2M gene. In certain embodiments, the first transgene comprises a polynucleotide coding for B2M fusion protein, such as a B2M-HLA-1 subunit fusion protein. In certain embodiments, the HLA-1 subunit comprises HLA-C, HLA-E, or HLA-G, preferably HLA-E or HLA-G. In a preferred embodiment the subunit is HLA-E. In a more preferred embodiment, the subunit is HLA-G. Additionally or alternatively, the cell can comprise a transgene comprising a polynucleotide coding for a CAR or portion thereof. In a preferred embodiment, the transgene comprising a polynucleotide coding for a CAR or portion thereof is inserted into the gene that codes for the subunit of a TCR protein, e.g., a TRAC gene. In certain embodiments, the transgene comprises a polynucleotide coding for a dual CAR or portions thereof, e.g., a CAR or portion thereof fusion protein. In certain embodiments, the dual CAR comprises a first CAR or portion thereof and a second CAR or portion thereof, wherein the second CAR or portion thereof is different from the first CAR or portion thereof. In certain embodiments, the CAR or portion thereof comprises a polypeptide that binds at least one of B7H3, BCMA, GPRC5D, CD8, CD8a, CD19, CD20, CD22, CD28, 4-1BB, or CD3zeta. In a preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-124. In a more preferred embodiment, the CAR or portion thereof comprises a polypeptide that binds at least one of B7H3, BCMxA, GPRC5D, CD8, CD8a, CD20, CD22, CD28, 4-1BB, or CD3zeta. In an even more preferred embodiment, the CAR or portion thereof comprises a polypeptide at least 60, at least 70, at least 80, at least 90, at least 95, at least 99%, or 100% identical to any one of the amino acid sequences of SEQ ID NOS: 86-104 or 116-124.

d. Cells Comprising Modifications that Result in Partial or Complete Inactivation of a Gene Coding for a Subunit of HLA-1 and TCR

In certain embodiments, provided herein are compositions comprising a cell comprising a first genomic modification in a gene that codes for a subunit of an HLA-1 protein, as described above, and a second genomic modification in a gene that codes for a subunit of a TCR protein. In certain embodiments, the first genomic modification partially or completely inactivates the gene that codes for a subunit of an HLA-1 protein and/or the second genomic modification partially or completely inactivates the gene that codes for a subunit of a TCR protein. In certain embodiments, the first genomic modification completely inactivates the gene that codes for a subunit of an HLA-1 protein and/or the second genomic modification partially or completely inactivates the gene that codes for a subunit of a TCR protein. In certain embodiments, the first and/or second genomic modification reduces or eliminates surface expression of active (immunogenic) HLA-1 and/or TCR proteins. In certain embodiments, the first and/or second genomic modifications completely eliminate surface expression of active (immunogenic) HLA- and/or TCR proteins. In certain embodiments, the gene that codes for a subunit of an HLA-1 protein comprises a B2M gene. In certain embodiments, the subunit of a TCR protein comprises an alpha or a beta subunit. In certain embodiments, the subunit of a TCR protein is a TRAC, TRBC, CD3E, CD3D, CD3G, or CD3Z protein. In certain embodiments, the subunit of a TCR protein comprises an alpha subunit. In certain embodiment, the gene that codes for a subunit of a TCR protein comprises a TRAC gene. In certain embodiments, the first and/or second genomic modification comprises a substitution, an insertion, a deletion, a nonsense mutation, a truncation, or a combination thereof. In certain embodiments, the first genomic modification comprises insertion of heterologous DNA, e.g., a transgene, for example a transgene comprising a polynucleotide coding for a CAR protein or a dual CAR protein.

In certain embodiments, the cell is a human cell, such as human stem cell or human immune cell, such as an immune cell comprising a neutrophil, cosinophil, basophil, mast cell, monocyte, macrophage, dendritic cell, natural killer cell, or a lymphocyte. In a preferred embodiment, the human immune cell is a T cell. In certain embodiments, the T cell comprises a chimeric antigen receptor (CAR) T cell. In certain embodiments, the CAR T cell expresses a plurality of different CARs, e.g., two different CARs (dual CAR T cell). In certain embodiments, the human cell is a human stem cell comprising a human pluripotent stem cell, multipotent stem cell, embryonic stem cell, induced pluripotent stem cell, hematopoietic stem cell, or a CD34+ cell. In a preferred embodiment, the cell is a hematopoietic stem cell. In a more preferred embodiment, the cell is a CD34+ stem cell. In an even more preferred embodiment, the cell is an induced pluripotent stem cell (iPSC).

In certain embodiments, the cell further comprises one or more nucleic acid-guided nucleases, one or more guide nucleic acids, and/or one or more polynucleotides encoding the one or more nucleic acid-guided nucleases and/or guide nucleic acids. In a preferred embodiment, the cell comprises a nucleic acid-guided nuclease complexed with a gRNA. In certain embodiments, one or more of the nucleic acid-guided nucleases (see Cas nucleases section below) are complexed with one or more of the guide nucleic acids (see Guide nucleic acids section below). In certain embodiments, the nuclease comprises a Type V nuclease. In a preferred embodiment, the nuclease comprises a Type V-A nuclease. In an even more preferred embodiment, the nuclease comprises MAD7, e.g., MAD7 comprising one or more nuclear localization signals (NLS), for example one to four NLS, preferably four NLS, more preferably one N-terminal NLS and three C-terminal NLS. In certain embodiments, the cell further comprises a donor template, such as a donor template described herein, e.g., a donor template comprising a polynucleotide coding for one or more CARs or portions thereof.

In certain embodiments, the cell further comprises a third genomic modification comprising a first transgene inserted into the genome. The first transgene can be inserted into any suitable location in the genome of the cell. In certain embodiments, the first transgene is inserted into a safe harbor site. The safe harbor site can be any suitable safe harbor site (see Genomic safe harbors section below). In certain embodiments, the safe harbor site comprises an AAVS1 or Rosa 26 locus. In certain embodiments the safe harbor site comprises any one of SEQ ID NOs: 2020-2043. In preferred embodiments, the first transgene is inserted into the gene that codes for the subunit of an HLA-1 protein, e.g., a B2M gene. In certain embodiments, the first transgene comprises a polynucleotide coding for B2M fusion protein, such as a B2M-HLA-1 subunit fusion protein. In certain embodiments, the HLA-1 subunit comprises HLA-C, HLA-E, or HLA-G, preferably HLA-E or HLA-G. In a preferred embodiment the subunit is HLA-E. In a more preferred embodiment, the subunit is HLA-G. Additionally or alternatively, the cell can comprise a transgene comprising a polynucleotide coding for a CAR or portion thereof. In a preferred embodiment, the transgene comprising a polynucleotide coding for a CAR or portion thereof is inserted into the gene that codes for the subunit of a TCR protein, e.g., a TRAC gene. In certain embodiments, the transgene comprises a polynucleotide coding for a dual CAR or portions thereof, e.g., a CAR or portion thereof fusion protein. In certain embodiments, the dual CAR comprises a first CAR or portion thereof and a second CAR or portion thereof, wherein the second CAR or portion thereof is different from the first CAR or portion thereof. In certain embodiments, the CAR or portion thereof comprises a polypeptide that binds at least one of B7H3, BCMA, GPRC5D, CD8, CD8a, CD19, CD20, CD22, CD28, 4-1BB, or CD3zeta. In a preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-124. In a more preferred embodiment, the CAR or portion thereof comprises a polypeptide that binds at least one of B7H3, BCMxA, GPRC5D, CD8, CD8a, CD20, CD22, CD28, 4-1BB, or CD3zeta. In an even more preferred embodiment, the CAR or portion thereof comprises a polypeptide at least 60, at least 70, at least 80, at least 90, at least 95, at least 99%, or 100% identical to any one of the amino acid sequences of SEQ ID NOS: 86-104 or 116-124.

e. Cells Comprising Modifications that Result in Partial or Complete Inactivation of a Gene Coding for a Subunit of HLA-2

In certain embodiments, provided herein are compositions comprising a cell comprising a first genomic modification in a gene that codes for a subunit of an HLA-2 protein or a transcription factor regulating the expression of one or more subunits of an HLA-2 protein. In certain embodiments, the first genomic modification partially or completely inactivates the gene that codes for a subunit of an HLA-2 protein or a transcription factor regulating the expression of one or more subunits of an HLA-2 protein. In certain embodiments, the first genomic modification completely inactivates the gene that codes for a subunit of an HLA-2 protein or a transcription factor regulating the expression of one or more subunits of an HLA-2 protein. In certain embodiments, the first genomic modification reduces or eliminates surface expression of active (immunogenic) HLA-2 proteins. In certain embodiments, the first genomic modification completely eliminates surface expression of active (immunogenic) HLA-2 proteins. In certain embodiments, the gene that codes for a transcription factor regulating the expression of one or more subunits of an HLA-2 protein comprises a CIITA gene. In certain embodiments, the first genomic modification comprises a substitution, an insertion, a deletion, a nonsense mutation, a truncation, or a combination thereof.

In certain embodiments, the cell is a human cell, such as human stem cell or human immune cell, such as an immune cell comprising a neutrophil, cosinophil, basophil, mast cell, monocyte, macrophage, dendritic cell, natural killer cell, or a lymphocyte. In a preferred embodiment, the human immune cell is a T cell. In certain embodiments, the T cell comprises a chimeric antigen receptor (CAR) T cell. In certain embodiments, the CAR T cell expresses a plurality of different CARS, e.g., two different CARs (dual CAR T cell). In certain embodiments, the human cell is a human stem cell comprising a human pluripotent stem cell, multipotent stem cell, embryonic stem cell, induced pluripotent stem cell, hematopoietic stem cell, or a CD34+ cell. In a preferred embodiment, the cell is a hematopoietic stem cell. In a more preferred embodiment, the cell is a CD34+ stem cell. In an even more preferred embodiment, the cell is an induced pluripotent stem cell (iPSC).

In certain embodiments, the cell further comprises one or more nucleic acid-guided nucleases, one or more guide nucleic acids, and/or one or more polynucleotides encoding the one or more nucleic acid-guided nucleases and/or guide nucleic acids. In a preferred embodiment, the cell comprises a nucleic acid-guided nuclease complexed with a gRNA. In certain embodiments, one or more of the nucleic acid-guided nucleases (see Cas nucleases section below) are complexed with one or more of the guide nucleic acids (see Guide nucleic acids section below). In certain embodiments, the nuclease comprises a Type V nuclease. In a preferred embodiment, the nuclease comprises a Type V-A nuclease. In an even more preferred embodiment, the nuclease comprises MAD7, e.g., MAD7 comprising one or more nuclear localization signals (NLS), for example one to four NLS, preferably four NLS, more preferably one N-terminal NLS and three C-terminal NLS. In certain embodiments, the cell further comprises a donor template, such as a donor template described herein, e.g., a donor template comprising a polynucleotide coding for one or more CARs or portions thereof.

In certain embodiments, the cell further comprises a second genomic modification comprising a first transgene inserted into the genome. The first transgene can be inserted into any suitable location in the genome of the cell. In certain embodiments, the first transgene is inserted into a safe harbor site. The safe harbor site can be any suitable safe harbor site (see Genomic safe harbors section below). In certain embodiments, the safe harbor site comprises an AAVS1 or Rosa 26 locus. In certain embodiments the safe harbor site comprises any one of SEQ ID NOS: 2020-2043. In certain embodiments, the first transgene comprises a polynucleotide coding for B2M fusion protein, such as a B2M-HLA-1 subunit fusion protein. In certain embodiments, the HLA-1 subunit comprises HLA-C, HLA-E, or HLA-G, preferably HLA-E or HLA-G. In a preferred embodiment the subunit is HLA-E. In a more preferred embodiment, the subunit is HLA-G. Additionally or alternatively, the cell can comprise a transgene comprising a polynucleotide coding for a CAR or portion thereof. In certain embodiments, the transgene comprises a polynucleotide coding for a dual CAR or portions thereof, e.g., a CAR or portion thereof fusion protein. In certain embodiments, the dual CAR comprises a first CAR or portion thereof and a second CAR or portion thereof, wherein the second CAR or portion thereof is different from the first CAR or portion thereof. In certain embodiments, the CAR or portion thereof comprises a polypeptide that binds at least one of B7H3, BCMA, GPRC5D, CD8, CD8a, CD19, CD20, CD22, CD28, 4-1BB, or CD3zeta. In a preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-124. In a more preferred embodiment, the CAR or portion thereof comprises a polypeptide that binds at least one of B7H3, BCMxA, GPRC5D, CD8, CD8a, CD20, CD22, CD28, 4-1BB, or CD3zeta. In an even more preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

f. Cells Comprising Modifications that Result in Partial or Complete Inactivation of a Gene Coding for a Subunit of HLA-2 and TCR

In certain embodiments, provided herein are compositions comprising a cell comprising a first genomic modification in a gene that codes for a subunit of an HLA-2 protein or a transcription factor regulating the expression of one or more subunits of an HLA-2 protein, as described above, and a second genomic modification in a gene that codes for a subunit of a TCR protein. In certain embodiments, the first genomic modification partially or completely inactivates the gene that codes for a subunit of an HLA-2 protein or a transcription factor regulating the expression of one or more subunits of an HLA-2 protein, and/or the second genomic modification partially or completely inactivates the gene that codes for a subunit of a TCR protein. In certain embodiments, the first genomic modification completely inactivates the gene that codes for a subunit of an HLA-2 protein or a transcription factor regulating the expression of one or more subunits of an HLA-2 protein, and/or the second genomic modification completely inactivates the gene that codes for a subunit of a TCR protein. In certain embodiments, the first and/or second genomic modification reduces or eliminates surface expression of active (immunogenic) HLA-2 and/or TCR proteins. In certain embodiments, the first and/or second genomic modification completely eliminates surface expression of active (immunogenic) HLA-2 and/or TCR proteins. In certain embodiments, the gene that codes for a transcription factor regulating the expression of one or more subunits of an HLA-2 protein comprises a CIITA gene. In certain embodiments, the subunit of a TCR protein comprises an alpha or a beta subunit. In certain embodiments, the subunit of a TCR protein is a TRAC, TRBC, CD3E, CD3D, CD3G, or CD3Z protein. In certain embodiments, the subunit of a TCR protein comprises an alpha subunit. In certain embodiment, the gene that codes for a subunit of a TCR protein comprises a TRAC gene. In certain embodiments, the first genomic modification comprises a substitution, an insertion, a deletion, a nonsense mutation, a truncation, or a combination thereof. In certain embodiments, the second genomic modification comprises insertion of heterologous DNA, e.g., a transgene, for example a polynucleotide coding for a CAR protein or a dual CAR protein.

In certain embodiments, the cell is a human cell, such as human stem cell or human immune cell, such as an immune cell comprising a neutrophil, cosinophil, basophil, mast cell, monocyte, macrophage, dendritic cell, natural killer cell, or a lymphocyte. In a preferred embodiment, the human immune cell is a T cell. In certain embodiments, the T cell comprises a chimeric antigen receptor (CAR) T cell. In certain embodiments, the CAR T cell expresses a plurality of different CARs, e.g., two different CARs (dual CAR T cell). In certain embodiments, the human cell is a human stem cell comprising a human pluripotent stem cell, multipotent stem cell, embryonic stem cell, induced pluripotent stem cell, hematopoietic stem cell, or a CD34+ cell. In a preferred embodiment, the cell is a hematopoietic stem cell. In a more preferred embodiment, the cell is a CD34+ stem cell. In an even more preferred embodiment, the cell is an induced pluripotent stem cell (iPSC).

In certain embodiments, the cell further comprises one or more nucleic acid-guided nucleases, one or more guide nucleic acids, and/or one or more polynucleotides encoding the one or more nucleic acid-guided nucleases and/or guide nucleic acids. In a preferred embodiment, the cell comprises a nucleic acid-guided nuclease complexed with a gRNA. In certain embodiments, one or more of the nucleic acid-guided nucleases (see Cas nucleases section below) are complexed with one or more of the guide nucleic acids (see Guide nucleic acids section below). In certain embodiments, the nuclease comprises a Type V nuclease. In a preferred embodiment, the nuclease comprises a Type V-A nuclease. In an even more preferred embodiment, the nuclease comprises MAD7, e.g., MAD7 comprising one or more nuclear localization signals (NLS), for example one to four NLS, preferably four NLS, more preferably one N-terminal NLS and three C-terminal NLS. In certain embodiments, the cell further comprises a donor template, such as a donor template described herein, e.g., a donor template comprising a polynucleotide coding for one or more CARs or portions thereof.

In certain embodiments, the cell further comprises a second genomic modification comprising a first transgene inserted into the genome. The first transgene can be inserted into any suitable location in the genome of the cell. In certain embodiments, the first transgene is inserted into a safe harbor site. The safe harbor site can be any suitable safe harbor site (see Genomic safe harbors section below). In certain embodiments, the safe harbor site comprises an AAVS1 or Rosa 26 locus. In certain embodiments the safe harbor site comprises any one of SEQ ID NOS: 2020-2043. In certain embodiments, the first transgene comprises a polynucleotide coding for B2M fusion protein, such as a B2M-HLA-1 subunit fusion protein. In certain embodiments, the HLA-1 subunit comprises HLA-C, HLA-E, or HLA-G, preferably HLA-E or HLA-G. In a preferred embodiment the subunit is HLA-E. In a more preferred embodiment, the subunit is HLA-G. Additionally or alternatively, the cell can comprise a transgene comprising a polynucleotide coding for a CAR or portion thereof. In certain embodiments, the first transgene is inserted into a TRAC gene. In certain embodiments, the transgene comprises a polynucleotide coding for a dual CAR or portions thereof, e.g., a CAR or portion thereof fusion protein. In certain embodiments, the dual CAR comprises a first CAR or portion thereof and a second CAR or portion thereof, wherein the second CAR or portion thereof is different from the first CAR or portion thereof. In certain embodiments, the CAR or portion thereof comprises a polypeptide that binds at least one of B7H3, BCMA, GPRC5D, CD8, CD8a, CD19, CD20, CD22, CD28, 4-1BB, or CD3zeta. In a preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-124. In a more preferred embodiment, the CAR or portion thereof comprises a polypeptide that binds at least one of B7H3, BCMxA, GPRC5D, CD8, CD8a, CD20, CD22, CD28, 4-1BB, or CD3zeta. In an even more preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

g. Cells Comprising Modifications that Result in Partial or Complete Inactivation of a Gene Coding for a Subunit of TCR

In certain embodiments, provided herein are compositions comprising a cell comprising a first genomic modification in a gene that codes for a subunit of a TCR protein. In certain embodiments, the first genomic modification partially or completely inactivates the gene that codes for a subunit of a TCR protein. In certain embodiments, the first genomic modification completely inactivates the gene that codes for a subunit of a TCR protein. In certain embodiments, the first genomic modification reduces or eliminates surface expression of active (immunogenic) TCR proteins. In certain embodiments, the first genomic modification completely eliminates surface expression of active (immunogenic) TCR proteins. In certain embodiments, the subunit of a TCR protein comprises an alpha or a beta subunit. In certain embodiments, the subunit of a TCR protein is a TRAC, TRBC, CD3E, CD3D, CD3G, or CD3Z protein. In certain embodiments, the subunit of a TCR protein comprises an alpha subunit. In certain embodiment, the gene that codes for a subunit of a TCR protein comprises a TRAC gene. In certain embodiments, the first genomic modification comprises a substitution, an insertion, a deletion, a nonsense mutation, a truncation, or a combination thereof. In certain embodiments, the first genomic modification comprises insertion of heterologous DNA, e.g., a transgene, for example a polynucleotide coding for a CAR protein or a dual CAR protein.

In certain embodiments, the cell is a human cell, such as human stem cell or human immune cell, such as an immune cell comprising a neutrophil, cosinophil, basophil, mast cell, monocyte, macrophage, dendritic cell, natural killer cell, or a lymphocyte. In a preferred embodiment, the human immune cell is a T cell. In certain embodiments, the T cell comprises a chimeric antigen receptor (CAR) T cell. In certain embodiments, the CAR T cell expresses a plurality of different CARS, e.g., two different CARs (dual CAR T cell). In certain embodiments, the human cell is a human stem cell comprising a human pluripotent stem cell, multipotent stem cell, embryonic stem cell, induced pluripotent stem cell, hematopoietic stem cell, or a CD34+ cell. In a preferred embodiment, the cell is a hematopoietic stem cell. In a more preferred embodiment, the cell is a CD34+ stem cell. In an even more preferred embodiment, the cell is an induced pluripotent stem cell (iPSC).

In certain embodiments, the cell further comprises one or more nucleic acid-guided nucleases, one or more guide nucleic acids, and/or one or more polynucleotides encoding the one or more nucleic acid-guided nucleases and/or guide nucleic acids. In a preferred embodiment, the cell comprises a nucleic acid-guided nuclease complexed with a gRNA. In certain embodiments, one or more of the nucleic acid-guided nucleases (see Cas nucleases section below) are complexed with one or more of the guide nucleic acids (see Guide nucleic acids section below). In certain embodiments, the nuclease comprises a Type V nuclease. In a preferred embodiment, the nuclease comprises a Type V-A nuclease. In an even more preferred embodiment, the nuclease comprises MAD7, e.g., MAD7 comprising one or more nuclear localization signals (NLS), for example one to four NLS, preferably four NLS, more preferably one N-terminal NLS and three C-terminal NLS. In certain embodiments, the cell further comprises a donor template, such as a donor template described herein, e.g., a donor template comprising a polynucleotide coding for one or more CARs or portions thereof.

In certain embodiments, the cell further comprises a second genomic modification comprising a first transgene inserted into the genome. The first transgene can be inserted into any suitable location in the genome of the cell. In certain embodiments, the first transgene is inserted into a safe harbor site. The safe harbor site can be any suitable safe harbor site (see Genomic safe harbors section below). In certain embodiments, the safe harbor site comprises an AAVS1 or Rosa 26 locus. In certain embodiments the safe harbor site comprises any one of SEQ ID NOS: 2020-2043. In certain embodiments, the first transgene comprises a polynucleotide coding for B2M fusion protein, such as a B2M-HLA-1 subunit fusion protein. In certain embodiments, the HLA-1 subunit comprises HLA-C, HLA-E, or HLA-G, preferably HLA-E or HLA-G. In a preferred embodiment the subunit is HLA-E. In a more preferred embodiment, the subunit is HLA-G. Additionally or alternatively, the cell can comprise a transgene comprising a polynucleotide coding for a CAR or portion thereof. In a preferred embodiment, the transgene comprising a polynucleotide coding for a CAR or portion thereof is inserted into the gene that codes for the subunit of a TCR protein, e.g., a TRAC gene. In certain embodiments, the transgene comprises a polynucleotide coding for a dual CAR or portions thereof, e.g., a CAR or portion thereof fusion protein. In certain embodiments, the dual CAR comprises a first CAR or portion thereof and a second CAR or portion thereof, wherein the second CAR or portion thereof is different from the first CAR or portion thereof. In certain embodiments, the CAR or portion thereof comprises a polypeptide that binds at least one of B7H3, BCMA, GPRC5D, CD8, CD8a, CD19, CD20, CD22, CD28, 4-1BB, or CD3zeta. In a preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-124. In a more preferred embodiment, the CAR or portion thereof comprises a polypeptide that binds at least one of B7H3, BCMxA, GPRC5D, CD8, CD8a, CD20, CD22, CD28, 4-1BB, or CD3zeta. In an even more preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

h. Surface Proteins & CARs

In certain embodiments, the surface expression of a cell comprising a genomic modification in a gene that codes for a subunit of an HLA-1, HLA-2, and/or TCR protein demonstrates no more than 90, 80, 70, 60, 50, 40, 30, 20, 19, 18, 17, 16, 15, 14, 13, 12, 11, 10, 9, 8, 7, 6, 5, 4, 3, 2, or 1% of active (immunogenic) protein as compared to an un-engineered equivalent, preferably no more than 20%, more preferably no more than 10%, even more preferably no more than 5%, yet more preferably no more than 2%. In certain embodiments, endogenous, surface expressed HLA-1 protein can be measured using any suitable technique. In certain embodiments, the technique comprises ELISA, proximity ligation assays, pull downs, and/or flow cytometry.

In certain embodiments, provided herein are compositions comprising CARs. In certain embodiments, the CAR or portion thereof comprises a polypeptide that binds at least one of B7H3, BCMA, GPRC5D, CD8, CD8a, CD19, CD20, CD22, CD28, 4-1BB, CD3zeta, or a combination thereof. In certain embodiments, the CAR or portion thereof comprises a polypeptide at least 60, at least 70, at least 80, at least 90, at least 95, at least 99%, or 100% identical to any one of the amino acid sequences of SEQ ID NOs: 86-124. In a preferred embodiment, the CAR or portion thereof comprises a polypeptide that binds at least one of B7H3, BCMxA, GPRC5D, CD8, CD8a, CD20, CD22, CD28, 4-1BB, or CD3zeta. In a preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-104 or 116-124. In certain embodiments, provided herein are composition comprising dual CARs comprising a first CAR or portion thereof and a second CAR or portion thereof, either separate, or connected via one or more polypeptide linkers. In certain embodiments where the dual CARs are separate, a first CAR or portion thereof can be inserted into a first suitable location in the genome and a second CAR or portion thereof can be inserted into a second suitable location in the genome and/or a polycistronic gene maybe be introduced into a suitable location in the genome comprising two or more CARs or portions thereof, wherein each CAR is expressed on the surface of the cell. In certain embodiments, the dual CAR comprises the same CAR polypeptide sequence. In a preferred embodiment, the dual CAR comprises different CAR polypeptide sequences.

TABLE 1
CARs

| SEQ | | |
|---|---|---|
| ID | | |
| NO | Antigen | Sequence |
| 86 | BCMA | EVQLVESGGGLVQPGGSLRLSCAASGNIFSDNLMGWFRQAPGKE |
| | | REFVAAINWNSRSTYYADSVKGRFTISADNSKNTAYLQMNSLKP |
| | | EDTAVYYCAKDLTMVRGVPDYWGQGTLVTVSS |
| 87 | BCMA | EVQLVESGGGLVQPGGSLRLSCAASGFTLGDYVMGWFRQAPGKE |
| | | REWVSVISSSGDFTSYADSVKGRFTISADNSKNTAYLQMNSLKP |
| | | EDTAVYYCASHYYDSSGTNWGQGTLVTVSS |
| 88 | BCMA | EVQLVESGGGLVQPGGSLRLSCAASGFTESSAIMGWFRQAPGKE |
| | | REFVSAITWNGTRTYYADSVKGRFTISADNSKNTAYLQMNSLKP |
| | | EDTAVYYCAKDLLEVGATPGNWGQGTLVTVSS |
| 89 | BCMA | EVQLLESGGGLVQPGGSLRLSCAASGFTFETYAMSWVRQAPGKG |
| | | LEWVSGISPSGGITTYADSVKGRFTISRDNSKNTLYLQMNSLRA |
| | | EDTAVYYCARREWWYDDWYLDYWGQGTLVTVSS |
| 90 | BCMA | EVQLLESGGGLVQPGGSLRLSCAASGFSFSTFAMSWVRQAPGKG |
| | | LEWVSAISGSGGSTSYADSVKGRFTISRDNSKNTLYLQMNSLRA |
| | | EDTAVYYCARRGWGSWSWYFDLWGQGTLVTVSS |
| 91 | BCMA | EVQLLESGGGLVQPGGSLRLSCAASGFTFGNYAMAWVRQAPGKG |
| | | LEWVSAISGSGGGTSYADSVKGRFTISRDNSKNTLYLQMNSLRA |
| | | EDTAVYYCARREWWYDDWYLDYWGQGTLVTVSS |
| 92 | BCMA | DIQMTQSPSSLSASVGDRVTITCRASQTIERRLNWYQQKPGKAP |
| | | KLLIYAASDLESGVPSRFSGSGSGTDFTLTISSLQPEDFATYYC |
| | | QQNNNWPTTFGQGTKVEIK |
| 93 | BCMA | DIQMTQSPSSLSASVGDRVTITCRASQTIGIYLNWYQQKPGKAP |
| | | KLLIYDASSLHSGVPSRFSGSGSGTDFTLTISSLQPEDFATYYC |
| | | QQSYSTPFTFGGGTKVEIK |
| 94 | BCMA | DIQMTQSPSSLSASVGDRVTITCRASQTIGDYLNWYQQKPGKAP |
| | | KLLIYAVTSRASGVPSRFSGSGSGTDFTLTISSLQPEDFATYYC |
| | | QQSYSTLTFGQGTKVEIK |
| 95 | B7H3 | EVQLVESGGGLVQPGGSLRLSCAASGIAFSIDIMGWFRQAPGKE |
| | | REFVAAVNWNGDSTYYADSVKGRFTISADNSKNTAYLQMNSLKP |
| | | EDTAVYYCATIDGSWREWGQGTLVTVSS |
| 96 | B7H3 | EVQLVESGGGLVQPGGSLRLSCAASGLREDDYWMGWFRQAPGKE |
| | | REFVSAINWSGVSTYYADSVKGRFTISADNSKNTAYLQMNSLKP |
| | | EDTAVYYCAARQYGEYWQAAGWGQGTLVTVSS |
| 97 | B7H3 | EVQLVESGGGLVQPGGSLRLSCAASGLTLDYYAMGWFRQAPGKE |
| | | REFVAGINNGRAITYYADSVKGRFTISADNSKNTAYLQMNSLKP |
| | | EDTAVYYCATIDGSWREWGQGTLVTVSS |
| 98 | B7H3 | EVQLLESGGGLVQPGGSLRLSCAASGFTFSNFPMSWVRQAPGKG |
| | | LEWVSAITGTGGSTYYADSVKGRFTISRDNSKNTLYLQMNSLRA |
| | | EDTAVYYCATRTGTTGTAFDIWGQGTLVTVSS |
| 99 | B7H3 | EVQLLESGGGLVQPGGSLRLSCAASGYTFSNYAMSWVRQAPGKG |
| | | LEWVSAVSRSGGSTYYADSVKGRFTISRDNSKNTLYLQMNSLRA |
| | | EDTAVYYCARDLGYYAFDFWGQGTLVTVSS |
| 100 | B7H3 | EVQLLESGGGLVQPGGSLRLSCAASGFTFSTYAMSWVRQAPGKG |
| | | LEWVSSISGSGGRTDYADSVKGRFTISRDNSKNTLYLQMNSLRA |
| | | EDTAVYYCARIRSRGSSGFDPWGQGTLVTVSS |
| 101 | B7H3 | DIQMTQSPSSLSASVGDRVTITCRASQNIGRYLNWYQQKPGKAP |
| | | KLLIYDASGLQSGVPSRFSGSGSGTDFTLTISSLQPEDFATYYC |
| | | QQSYSTPPWTFGGGTKVEIK |
| 102 | B7H3 | DIQMTQSPSSLSASVGDRVTITCRASQTIYRYLNWYQQKPGKAP |
| | | KLLIYHASNLQSGVPSRFSGSGSGTDFTLTISSLQPEDFATYYC |
| | | QQSYTFPRSFGGGTKVEIK |
| 103 | B7H3 | DIQMTQSPSSLSASVGDRVTITCRASQSVYSYLNWYQQKPGKAP |
| | | KLLIYETSNLQSGVPSRFSGSGSGTDFTLTISSLQPEDFATYYC |
| | | QQSFTSPLTFGGGTKVEIK |
| 104 | CD19 | EVQLLESGGGLVQPGGSLRLSCAASGFTFENYAMSWVRQAPGKG |
| | | LEWVSAISGSGGHTYYADSVKGRFTISRDNSKNTLYLQMNSLRA |
| | | EDTAVYYCAHSNKRTGHAFDIWGQGTLVTVSS |
| 105 | CD19 | EVQLLESGGGLVQPGGSLRLSCAASGFTFSRHAMSWVRQAPGKG |
| | | LEWVSAITGSGASTYYADSVKGRFTISRDNSKNTLYLQMNSLRA |
| | | EDTAVYYCARGGRREFHYGLDYWGQGTLVTVSS |
| 106 | CD19 | EVQLLESGGGLVQPGGSLRLSCAASGFTFGNYAMAWVRQAPGKG |
| | | LEWVSAISGNGGSTFYADSVKGRFTISRDNSKNTLYLQMNSLRA |
| | | EDTAVYYCARAGRILFDYWGQGTLVTVSS |
| 107 | CD19 | EVQLLESGGGLVQPGGSLRLSCAASGFTESTYAMSWVRQAPGKG |
| | | LEWVSAISRSGGNTYYADSVKGRFTISRDNSKNTLYLQMNSLRA |
| | | EDTAVYYCARVRMKGYTYFDPWGQGTLVTVSS |
| 108 | CD19 | EVQLLESGGGLVQPGGSLRLSCAASGFTFSHYGMSWVRQAPGKG |
| | | LEWVSSISGSGGSTYYVDSVKGRFTISRDNSKNTLYLQMNSLRA |
| | | EDTAVYYCARSKRLIHGLDVWGQGTLVTVSS |
| 109 | CD19 | EVQLLESGGGLVQPGGSLRLSCAASGFTFSRYTMSWVRQAPGKG |
| | | LEWVSTISGSGYSTYYADSVKGRFTISRDNSKNTLYLQMNSLRA |
| | | EDTAVYYCAHSNKRTGHAFDIWGQGTLVTVSS |
| 110 | CD19 | DIQMTQSPSSLSASVGDRVTITCRASQSVSTFLNWYQQKPGKAP |
| | | KLLIYGASILQSGVPSRFSGSGSGTDFTLTISSLQPEDFATYYC |
| | | QQSYTPPLTFGGGTKVEIK |
| 111 | CD19 | DIQMTQSPSSLSASVGDRVTITCRASQSVSRFLNWYQQKPGKAP |
| | | KLLIYAASVLQSGVPSRFSGSGSGTDFTLTISSLQPEDFATYYC |
| | | QQTYSPPLTFGGGTKVEIK |
| 112 | CD19 | DIQMTQSPSSLSASVGDRVTITCRASQSIRRYLNWYQQKPGKAP |
| | | KLLIYHTSRLQSGVPSRFSGSGSGTDFTLTISSLQPEDFATYYC |
| | | AQGWGRPVTFGQGTKVEIK |
| 113 | CD19 | DIQMTQSPSSLSASVGDRVTITCRASQTISSSLNWYQQKPGKAP |
| | | KLLIYGASSLRSGVPSRFSGSGSGTDFTLTISSLQPEDFATYYC |
| | | QQTYSNPITFGGGTKVEIK |
| 114 | CD19 | DIQMTQSPSSLSASVGDRVTITCRTSQSISTYLNWYQQKPGKAP |
| | | KLLIYGASALQTGVPSRFSGSGSGTDFTLTISSLQPEDFATYYC |
| | | QQSYTAPLTFGGGTKVEIK |
| 115 | CD19 | DIQMTQSPSSLSASVGDRVTITCRASQTISKYLNWYQQKPGKAP |
| | | KLLIYGASSLQSGVPSRFSGSGSGTDFTLTISSLQPEDFATYYC |
| | | QQSYSPPITFGGGTKVEIK |
| 116 | CD22 | EVQLVESGGGLVQPGGSLRLSCAASGIPSIRAMGWFRQAPGKER |
| | | EWVSSINSDGTSAFYADSVKGRFTISADNSKNTAYLQMNSLKPE |
| | | DTAVYYCARAYGRGTYDWGQGTLVTVSS |
| 117 | CD22 | EVQLVESGGGLVQPGGSLRLSCAASGFTFGEYAMGWFRQAPGKE |
| | | REFVASISRSGTLRAYADSVKGRFTISADNSKNTAYLQMNSLKP |
| | | EDTAVYYCAKESKDYFYMDVWGQGTLVTVSS |
| 118 | CD22 | EVQLVESGGGLVQPGGSLRLSCAASGRTYGMGWFRQAPGKEREF |
| | | VASVTSGGYTNYADSVKGRFTISADNSKNTAYLQMNSLKPEDTA |
| | | VYYCARGGGTSVRAFDIWGQGTLVTVSS |
| 119 | CD22 | EVQLLESGGGLVQPGGSLRLSCAASGFAFAAYDMGWVRQAPGKG |
| | | LEWVSSISGYGSTTYYADSVKGRFTISRDNSKNTLYLQMNSLRA |
| | | EDTAVYYCARHSGYGSSYGVLFAYWGQGTLVTVSS |
| 120 | CD22 | EVQLLESGGGLVQPGGSLRLSCAASGFAFAAYDMGWVRQAPGKG |
| | | LEWVATISGGGINTYYPDSVKGRFTISRDNSKNTLYLQMNSLRA |
| | | EDTAVYYCARHSGYGSSYGVLFAYWGQGTLVTVSS |
| 121 | CD22 | EVQLLESGGGLVQPGGSLRLSCAASGFTFPVYNMAWVRQAPGKG |
| | | LEWVSEIDALGTDTYYADSVKGRFTISRDNSKNTLYLQMNSLRA |
| | | EDTAVYYCARHSGYGSSYGVLFAYWGQGTLVTVSS |
| 122 | CD22 | DIQMTQSPSSLSASVGDRVTITCRASQSISNNLNWYQQKPGKAP |
| | | KLLIYGKNIRPSGVPSRFSGSGSGTDFTLTISSLQPEDFATYYC |
| | | FQGSQFPYTFGQGTKVEIK |
| 123 | CD22 | DIQMTQSPSSLSASVGDRVTITCRASQDVSSGVAWYQQKPGKAP |
| | | KLLIYHASQSISGVPSRFSGSGSGTDFTLTISSLQPEDFATYYC |
| | | QSYDLKSLNVVFGQGTKVEIK |
| 124 | CD22 | DIQMTQSPSSLSASVGDRVTITCQASQSISSYLAWYQQKPGKAP |
| | | KLLIYGQHNRPSGVPSRFSGSGSGTDFTLTISSLQPEDFATYYC |
| | | QQSYNTPRTFGQGTKVEIK |

### 2. Cell Populations Comprising Genomic Modifications

In certain embodiments, provided herein are compositions comprising one or more populations of cells having genetic modifications as described in the Cells comprising Genomic modifications section above. In certain embodiments, the composition comprises a single cell population, wherein each of the cells comprises the same set of genomic modifications (1) through (3). In certain embodiments, provided herein are compositions comprising a plurality of cell populations, wherein each cell population comprise a different set of genomic modifications. In general, at least one cell population comprises cells that comprise all of (1) one or more genomic modifications that partially or completely inactivates one or more genes that codes for a subunit of an HLA-1 protein, (2) one or more genomic modifications that partially or completely inactivates one or more genes coding for a subunit of an HLA-2 protein or a transcription factor regulating the expression of one or more subunits of an HLA-2 protein, and (3) one or more genomic modifications that partially or completely inactivates one or more genes that codes for a subunit of a TCR protein, in addition to one or more additional cell populations that do not comprise all three genetic modifications. In certain embodiments, the one or more additional cell populations comprise cells comprising (1) one or more genomic modifications that partially or completely inactivates one or more genes that codes for a subunit of an HLA-1 protein, (2) one or more genomic modifications that partially or completely inactivates one or more genes coding for a subunit of an HLA-2 protein or a transcription factor regulating the expression of one or more subunits of an HLA-2 protein, and/or (3) one or more genomic modifications that partially or completely inactivates one or more genes that codes for a subunit of a TCR protein, but not all of (1)-(3). In a preferred embodiment, the subunit of an HLA-1 protein comprises B2M. In a preferred embodiment, the transcription factor regulating the expression of one or more subunits of an HLA-2 protein comprises CIITA. In certain embodiments, the subunit of a TCR protein is an alpha subunit or a beta subunit. In certain embodiments, the subunit of a TCR protein is a TRAC, TRBC, CD3E, CD3D, CD3G, or CD3Z protein. In a preferred embodiment, the gene that codes for a subunit of a TCR protein is a TRAC gene. In a more preferred embodiment, the at least one cell population comprising cells comprising all three genomic modifications comprises (1) one or more genomic modifications that partially or completely inactivates a B2M gene, (2) one or more genomic modifications that partially or completely inactivates a CIITA gene, and (3) one or more genomic modifications that partially or completely inactivates a TRAC gene. In certain embodiments, the plurality of cell populations comprise at least 1, 2, 3, 4, 5, 6, 7, 8, 9, 10, 11, 12, 13, 14, 15, 16, 17, 18, 19, 20, 25, 30, 35, 40, or 45 and/or no more than 2, 3, 4, 5, 6, 7, 8, 9, 10, 11, 12, 13, 14, 15, 16, 17, 18, 19, 20, 25, 30, 35, 40, 45, or 50 populations.

In certain embodiments, the first cell population comprises at least 1, 2, 3, 4, 5, 6, 7, 8, 9, 10, 15, 20, 25, 30, 35, 40, 45, 50, 55, 60, 65, or 70% and/or no more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations, preferably 5-75%, more preferably 10-75%, even more preferably 15-75%, yet even more preferably 20-75%. In certain embodiments, the second cell population comprises at least 1, 2, 3, 4, 5, 6, 7, 8, 9, 10, 15, 20, 25, 30, 35, 40, 45, 50, 55, 60, 65, or 70% and/or no more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations, preferably no more than 50%, more preferably no more that 30%, even more preferably no more than 20%, yet even more preferably no more than 10%. In certain embodiments, the third cell population comprises at least 1, 2, 3, 4, 5, 6, 7, 8, 9, 10, 15, 20, 25, 30, 35, 40, 45, 50, 55, 60, 65, or 70% and/or no more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations preferably no more than 50%, more preferably no more that 30%, even more preferably no more than 20%, yet even more preferably no more than 10%. In certain embodiments, the fourth cell population comprises at least 1, 2, 3, 4, 5, 6, 7, 8, 9, 10, 15, 20, 25, 30, 35, 40, 45, 50, 55, 60, 65, or 70% and/or no more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations, preferably no more than 50%, more preferably no more that 30%, even more preferably no more than 20%, yet even more preferably no more than 10%. It is understood that the sum of the percentages for each cell population in the plurality adds to 100%.

The number, relative abundance, and/or identity of cell populations in a plurality of cell populations can be measured by any suitable method. In certain embodiments, the number, relative abundance, and/or identity of cell populations in a plurality of cell populations can be measured by analyzing one or more nucleic acids in a sample using one or more methods, for example PCR, multiplex PCR, FISH, and/or sequencing. In certain embodiments, the number and/or identity of cell populations in a plurality of cell populations can be measured by analyzing one or more cell surface proteins and/or lack thereof in a sample using one or more methods, for example immunostaining and microscopy, ELISA, pull downs, and/or flow cytometry.

### 3. Guide Nucleic Acids and Nucleic Acid-Guided Nuclease Complexes for Generating Genomic Modifications

In certain embodiments, provided herein are compositions comprising a guide nucleic acid, a nucleic acid-guided nuclease, a nucleic acid-guided nuclease complex, and/or one or more polynucleotides encoding thereof. In certain embodiments, the nucleic acid-guided nuclease, guide nucleic acid, and/or complex thereof further comprises a donor template. In certain embodiments, the nucleic acid-guided nuclease, guide nucleic acid, and/or complex thereof further comprises an additive that stabilizes the nucleic acid-guided nuclease complex. In certain embodiments, the nucleic acid-guided nuclease and/or guide nucleic acid are combined in the presence of an aqueous buffer. In certain embodiments, the nucleic acid-guided nuclease, guide nucleic acid, and/or complex thereof further comprises further comprise an excipient. In certain embodiments, the nucleic acid-guided nuclease, guide nucleic acid, and/or complex thereof are lyophilized, e.g., freeze-dried, with one or more excipient.

a. Compositions Comprising Guide Nucleic Acids Comprising a Spacer Sequence Directed at a Target Nucleotide Sequence in a Gene Coding for a Subunit of an HLA-1 Protein

In certain embodiments, provided herein are compositions comprising a first guide nucleic acid comprising a spacer sequence directed at a target nucleotide sequence in a gene coding for a subunit of an HLA-1 protein. In certain embodiments, the guide nucleic acid comprises a targeter nucleic acid and a modulator nucleic acid, wherein the targeter nucleic acid comprises a targeter nucleic acid comprising a targeter stem sequence and a spacer sequence, and the modulator nucleic acid comprises a modulator stem sequence complementary to the targeter stem sequence, and, optionally, a 5′ sequence. The spacer sequence can be any suitable sequence. In certain embodiments, the spacer sequence comprises any one of SEQ ID NOs: 125-2019. In certain embodiments, the guide nucleic acid comprises a single polynucleotide. In preferred embodiments, the guide nucleic acid comprises a dual guide nucleic acid (as described in the Guide nucleic acids section below), wherein the targeter nucleic acid and the modulator nucleic acid are separate polynucleotides. In certain embodiments, the guide nucleic acid comprises one or more chemical modifications to one or more nucleotides and/or internucleotide linkages at or near the 5′ end, the 3′ end, and/or both as described in the gNA modifications section below.

In certain embodiments, the guide nucleic acid further comprises a nucleic acid-guided nuclease. The guide nucleic acid can be combined and/or complexed with any suitable nucleic acid-guided nuclease. In certain embodiments, the nucleic acid-guided comprises a Type V CRISPR endonuclease, preferably MAD2, MAD7, ART2, ART11, and/or ART11*, more preferably MAD7.

In certain embodiments, the guide nucleic acid further comprises a nucleic acid-guided nuclease. Any suitable donor template can be combined with the guide nucleic acid. In certain embodiments, the guide nucleic acid comprises a donor template as described in the Donor templates section below. In certain embodiments, the donor template comprises a transgene. In preferred embodiments, the transgene comprises a polynucleotide coding for B2M fusion protein, such as a B2M-HLA-1 subunit fusion protein. In certain embodiments, the HLA-1 subunit comprises HLA-C, HLA-E, or HLA-G, preferably HLA-E or HLA-G. In a preferred embodiment the subunit is HLA-E. In a more preferred embodiment, the subunit is HLA-G. Additionally or alternatively, the cell can comprise a transgene comprising a polynucleotide coding for a CAR or portion thereof. In certain embodiments, the transgene comprises a polynucleotide coding for a dual CAR or portions thereof, e.g., a CAR or portion thereof fusion protein. In certain embodiments, the dual CAR comprises a first CAR or portion thereof and a second CAR or portion thereof, wherein the second CAR or portion thereof is different from the first CAR or portion thereof. In certain embodiments, the CAR or portion thereof comprises a polypeptide that binds at least one of B7H3, BCMA, GPRC5D, CD8, CD8a, CD19, CD20, CD22, CD28, 4-1BB, or CD3zeta. In a preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-124. In a more preferred embodiment, the CAR or portion thereof comprises a polypeptide that binds at least one of B7H3, BCMxA, GPRC5D, CD8, CD8a, CD20, CD22, CD28, 4-1BB, or CD3zeta. In an even more preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

donor template can further comprise a cell. The cell can be any suitable cell. In certain embodiments, the cell is a human cell, such as human stem cell or human immune cell, such as an immune cell comprising a neutrophil, cosinophil, basophil, mast cell, monocyte, macrophage, dendritic cell, natural killer cell, or a lymphocyte. In a preferred embodiment, the human immune cell is a T cell. In certain embodiments, the T cell comprises a chimeric antigen receptor (CAR) T cell. In certain embodiments, the CAR T cell expresses a plurality of different CARs, e.g., two different CARs (dual CAR T cell). In certain embodiments, the human cell is a human stem cell comprising a human pluripotent stem cell, multipotent stem cell, embryonic stem cell, induced pluripotent stem cell, hematopoietic stem cell, or a CD34+ cell. In a preferred embodiment, the cell is a hematopoietic stem cell. In a more preferred embodiment, the cell is a CD34+ stem cell. In an even more preferred embodiment, the cell is an induced pluripotent stem cell (iPSC).

b. Compositions Comprising Guide Nucleic Acids Comprising a Spacer Sequence Directed at a Target Nucleotide Sequence in a Gene Coding for a Subunit of an HLA-1 Protein and/or a Gene Coding for a Subunit of an HLA-2 Protein or a Transcription Factor Regulating the Expression of One or More Subunits of an HLA-2 Protein

In certain embodiments, provided herein are compositions comprising a first guide nucleic acid comprising a spacer sequence directed at a target nucleotide sequence in a gene coding for a subunit of an HLA-1 protein, as described above, and a second guide nucleic acid comprising a spacer sequence directed at a target nucleotide sequence in a gene coding for a subunit of an HLA-2 protein or a transcription factor regulating the expression of one or more subunits of an HLA-2 protein. In certain embodiments, the guide nucleic acid comprises a targeter nucleic acid and a modulator nucleic acid, wherein the targeter nucleic acid comprises a targeter nucleic acid comprising a targeter stem sequence and a spacer sequence, and the modulator nucleic acid comprises a modulator stem sequence complementary to the targeter stem sequence, and, optionally, a 5′ sequence. The spacer sequence can be any suitable sequence. In certain embodiments, the spacer sequence comprises any one of SEQ ID NOs: 125-2019. In certain embodiments, the guide nucleic acid comprises a single polynucleotide. In preferred embodiments, the guide nucleic acid comprises a dual guide nucleic acid (as described in the Guide nucleic acids section below), wherein the targeter nucleic acid and the modulator nucleic acid are separate polynucleotides. In certain embodiments, the guide nucleic acid comprises one or more chemical modifications to one or more nucleotides and/or internucleotide linkages at or near the 5′ end, the 3′ end, and/or both as described in the gNA modifications section below.

In certain embodiments, the guide nucleic acid further comprises a nucleic acid-guided nuclease. The guide nucleic acid can be combined and/or complexed with any suitable nucleic acid-guided nuclease. In certain embodiments, the nucleic acid-guided comprises a Type V CRISPR endonuclease, preferably MAD2, MAD7, ART2, ART11, and/or ART11*, more preferably MAD7.

In certain embodiments, the guide nucleic acid further comprises a nucleic acid-guided nuclease. Any suitable donor template can be combined with the guide nucleic acid. In certain embodiments, the guide nucleic acid comprises a donor template as described in the Donor templates section below. In certain embodiments, the donor template comprises a transgene. In preferred embodiments, the transgene comprises a polynucleotide coding for B2M fusion protein, such as a B2M-HLA-1 subunit fusion protein. In certain embodiments, the HLA-1 subunit comprises HLA-C, HLA-E, or HLA-G, preferably HLA-E or HLA-G. In a preferred embodiment the subunit is HLA-E. In a more preferred embodiment, the subunit is HLA-G. Additionally or alternatively, the cell can comprise a transgene comprising a polynucleotide coding for a CAR or portion thereof. In certain embodiments, the transgene comprises a polynucleotide coding for a dual CAR or portions thereof, e.g., a CAR or portion thereof fusion protein. In certain embodiments, the dual CAR comprises a first CAR or portion thereof and a second CAR or portion thereof, wherein the second CAR or portion thereof is different from the first CAR or portion thereof. In certain embodiments, the CAR or portion thereof comprises a polypeptide that binds at least one of B7H3, BCMA, GPRC5D, CD8, CD8a, CD19, CD20, CD22, CD28, 4-1BB, or CD3zeta. In a preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-124. In a more preferred embodiment, the CAR or portion thereof comprises a polypeptide that binds at least one of B7H3, BCMxA, GPRC5D, CD8, CD8a, CD20, CD22, CD28, 4-1BB, or CD3zeta. In an even more preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

In certain embodiments, the guide nucleic acid, nucleic acid-guided nuclease, and/or donor template can further comprise a cell. The cell can be any suitable cell. In certain embodiments, the cell is a human cell, such as human stem cell or human immune cell, such as an immune cell comprising a neutrophil, cosinophil, basophil, mast cell, monocyte, macrophage, dendritic cell, natural killer cell, or a lymphocyte. In a preferred embodiment, the human immune cell is a T cell. In certain embodiments, the T cell comprises a chimeric antigen receptor (CAR) T cell. In certain embodiments, the CAR T cell expresses a plurality of different CARs, e.g., two different CARs (dual CAR T cell). In certain embodiments, the human cell is a human stem cell comprising a human pluripotent stem cell, multipotent stem cell, embryonic stem cell, induced pluripotent stem cell, hematopoietic stem cell, or a CD34+ cell. In a preferred embodiment, the cell is a hematopoietic stem cell. In a more preferred embodiment, the cell is a CD34+ stem cell. In an even more preferred embodiment, the cell is an induced pluripotent stem cell (iPSC).

c. Compositions Comprising Guide Nucleic Acids Comprising a Spacer Sequence Directed at a Target Nucleotide Sequence in a Gene Coding for a Subunit of an HLA-1 Protein, a Gene Coding for a Subunit of an HLA-2 Protein or a Transcription Factor Regulating the Expression of One or More Subunits of an HLA-2 Protein, and/or a Gene Coding for a Subunit of an TCR Protein

In certain embodiments, provided herein are compositions comprising a first guide nucleic acid comprising a spacer sequence directed at a target nucleotide sequence in a gene coding for a subunit of an HLA-1 protein, a second guide nucleic acid comprising a spacer sequence directed at a target nucleotide sequence in a gene coding for a subunit of an HLA-2 protein or a transcription factor regulating the expression of one or more subunits of an HLA-2 protein, as described above, and a third guide nucleic acid directed at a target nucleotide sequence in a gene coding for a subunit of a TCR protein. In certain embodiments, the guide nucleic acid comprises a targeter nucleic acid and a modulator nucleic acid, wherein the targeter nucleic acid comprises a targeter nucleic acid comprising a targeter stem sequence and a spacer sequence, and the modulator nucleic acid comprises a modulator stem sequence complementary to the targeter stem sequence, and, optionally, a 5′ sequence. The spacer sequence can be any suitable sequence. In certain embodiments, the spacer sequence comprises any one of SEQ ID NOs: 125-2019. In certain embodiments, the guide nucleic acid comprises a single polynucleotide. In preferred embodiments, the guide nucleic acid comprises a dual guide nucleic acid (as described in the Guide nucleic acids section below), wherein the targeter nucleic acid and the modulator nucleic acid are separate polynucleotides. In certain embodiments, the guide nucleic acid comprises one or more chemical modifications to one or more nucleotides and/or internucleotide linkages at or near the 5′ end, the 3′ end, and/or both as described in the gNA modifications section below.

In certain embodiments, the guide nucleic acid further comprises a nucleic acid-guided nuclease. The guide nucleic acid can be combined and/or complexed with any suitable nucleic acid-guided nuclease. In certain embodiments, the nucleic acid-guided comprises a Type V CRISPR endonuclease, preferably MAD2, MAD7, ART2, ART11, and/or ART11*, more preferably MAD7.

In certain embodiments, the guide nucleic acid further comprises a nucleic acid-guided nuclease. Any suitable donor template can be combined with the guide nucleic acid. In certain embodiments, the guide nucleic acid comprises a donor template as described in the Donor templates section below. In certain embodiments, the donor template comprises a transgene. In preferred embodiments, the transgene comprises a polynucleotide coding for B2M fusion protein, such as a B2M-HLA-1 subunit fusion protein. In certain embodiments, the HLA-1 subunit comprises HLA-C, HLA-E, or HLA-G, preferably HLA-E or HLA-G. In a preferred embodiment the subunit is HLA-E. In a more preferred embodiment, the subunit is HLA-G. Additionally or alternatively, the cell can comprise a transgene comprising a polynucleotide coding for a CAR or portion thereof. In certain embodiments, the transgene comprises a polynucleotide coding for a dual CAR or portions thereof, e.g., a CAR or portion thereof fusion protein. In certain embodiments, the dual CAR comprises a first CAR or portion thereof and a second CAR or portion thereof, wherein the second CAR or portion thereof is different from the first CAR or portion thereof. In certain embodiments, the CAR or portion thereof comprises a polypeptide that binds at least one of B7H3, BCMA, GPRC5D, CD8, CD8a, CD19, CD20, CD22, CD28, 4-1BB, or CD3zeta. In a preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-124. In a more preferred embodiment, the CAR or portion thereof comprises a polypeptide that binds at least one of B7H3, BCMxA, GPRC5D, CD8, CD8a, CD20, CD22, CD28, 4-1BB, or CD3zeta. In an even more preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

In certain embodiments, the guide nucleic acid, nucleic acid-guided nuclease, and/or donor template can further comprise a cell. The cell can be any suitable cell. In certain embodiments, the cell is a human cell, such as human stem cell or human immune cell, such as an immune cell comprising a neutrophil, cosinophil, basophil, mast cell, monocyte, macrophage, dendritic cell, natural killer cell, or a lymphocyte. In a preferred embodiment, the human immune cell is a T cell. In certain embodiments, the T cell comprises a chimeric antigen receptor (CAR) T cell. In certain embodiments, the CAR T cell expresses a plurality of different CARs, e.g., two different CARs (dual CAR T cell). In certain embodiments, the human cell is a human stem cell comprising a human pluripotent stem cell, multipotent stem cell, embryonic stem cell, induced pluripotent stem cell, hematopoietic stem cell, or a CD34+ cell. In a preferred embodiment, the cell is a hematopoietic stem cell. In a more preferred embodiment, the cell is a CD34+ stem cell. In an even more preferred embodiment, the cell is an induced pluripotent stem cell (iPSC).

d. Compositions Comprising Guide Nucleic Acids Comprising a Spacer Sequence Directed at a Target Nucleotide Sequence in a Gene Coding for a Subunit of an HLA-1 Protein and/or a Gene Coding for a Subunit of an TCR Protein

In certain embodiments, provided herein are compositions comprising a first guide nucleic acid comprising a spacer sequence directed at a target nucleotide sequence in a gene coding for a subunit of an HLA-1 protein, as described above, and a second guide nucleic acid comprising a spacer sequence directed at a target nucleotide sequence in a gene coding for a subunit of a TCR protein. In certain embodiments, the guide nucleic acid comprises a targeter nucleic acid and a modulator nucleic acid, wherein the targeter nucleic acid comprises a targeter nucleic acid comprising a targeter stem sequence and a spacer sequence, and the modulator nucleic acid comprises a modulator stem sequence complementary to the targeter stem sequence, and, optionally, a 5′ sequence. The spacer sequence can be any suitable sequence. In certain embodiments, the spacer sequence comprises any one of SEQ ID NOs: 125-2019. In certain embodiments, the guide nucleic acid comprises a single polynucleotide. In preferred embodiments, the guide nucleic acid comprises a dual guide nucleic acid (as described in the Guide nucleic acids section below), wherein the targeter nucleic acid and the modulator nucleic acid are separate polynucleotides. In certain embodiments, the guide nucleic acid comprises one or more chemical modifications to one or more nucleotides and/or internucleotide linkages at or near the 5′ end, the 3′ end, and/or both as described in the gNA modifications section below.

In certain embodiments, the guide nucleic acid further comprises a nucleic acid-guided nuclease. The guide nucleic acid can be combined and/or complexed with any suitable nucleic acid-guided nuclease. In certain embodiments, the nucleic acid-guided comprises a Type V CRISPR endonuclease, preferably MAD2, MAD7, ART2, ART11, and/or ART11*, more preferably MAD7.

In certain embodiments, the guide nucleic acid further comprises a nucleic acid-guided nuclease. Any suitable donor template can be combined with the guide nucleic acid. In certain embodiments, the guide nucleic acid comprises a donor template as described in the Donor templates section below. In certain embodiments, the donor template comprises a transgene. In preferred embodiments, the transgene comprises a polynucleotide coding for B2M fusion protein, such as a B2M-HLA-1 subunit fusion protein. In certain embodiments, the HLA-1 subunit comprises HLA-C, HLA-E, or HLA-G, preferably HLA-E or HLA-G. In a preferred embodiment the subunit is HLA-E. In a more preferred embodiment, the subunit is HLA-G. Additionally or alternatively, the cell can comprise a transgene comprising a polynucleotide coding for a CAR or portion thereof. In certain embodiments, the transgene comprises a polynucleotide coding for a dual CAR or portions thereof, e.g., a CAR or portion thereof fusion protein. In certain embodiments, the dual CAR comprises a first CAR or portion thereof and a second CAR or portion thereof, wherein the second CAR or portion thereof is different from the first CAR or portion thereof. In certain embodiments, the CAR or portion thereof comprises a polypeptide that binds at least one of B7H3, BCMA, GPRC5D, CD8, CD8a, CD19, CD20, CD22, CD28, 4-1BB, or CD3zeta. In a preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-124. In a more preferred embodiment, the CAR or portion thereof comprises a polypeptide that binds at least one of B7H3, BCMxA, GPRC5D, CD8, CD8a, CD20, CD22, CD28, 4-1BB, or CD3zeta. In an even more preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

In certain embodiments, the guide nucleic acid, nucleic acid-guided nuclease, and/or donor template can further comprise a cell. The cell can be any suitable cell. In certain embodiments, the cell is a human cell, such as human stem cell or human immune cell, such as an immune cell comprising a neutrophil, cosinophil, basophil, mast cell, monocyte, macrophage, dendritic cell, natural killer cell, or a lymphocyte. In a preferred embodiment, the human immune cell is a T cell. In certain embodiments, the T cell comprises a chimeric antigen receptor (CAR) T cell. In certain embodiments, the CAR T cell expresses a plurality of different CARs, e.g., two different CARs (dual CAR T cell). In certain embodiments, the human cell is a human stem cell comprising a human pluripotent stem cell, multipotent stem cell, embryonic stem cell, induced pluripotent stem cell, hematopoietic stem cell, or a CD34+ cell. In a preferred embodiment, the cell is a hematopoietic stem cell. In a more preferred embodiment, the cell is a CD34+ stem cell. In an even more preferred embodiment, the cell is an induced pluripotent stem cell (iPSC).

e. Compositions Comprising Guide Nucleic Acids Comprising a Spacer Sequence Directed at a Target Nucleotide Sequence in a Gene Coding for a Subunit of an HLA-2 Protein or a Transcription Factor Regulating the Expression of One or More Subunits of an HLA-2 Protein

In certain embodiments, provided herein are compositions comprising a first guide nucleic acid comprising a spacer sequence directed at a target nucleotide sequence in a gene coding for a subunit of an HLA-2 protein or a transcription factor regulating the expression of one or more subunits of an HLA-2 protein. In certain embodiments, the guide nucleic acid comprises a targeter nucleic acid and a modulator nucleic acid, wherein the targeter nucleic acid comprises a targeter nucleic acid comprising a targeter stem sequence and a spacer sequence, and the modulator nucleic acid comprises a modulator stem sequence complementary to the targeter stem sequence, and, optionally, a 5′ sequence. The spacer sequence can be any suitable sequence. In certain embodiments, the spacer sequence comprises any one of SEQ ID NOs: 125-2019. In certain embodiments, the guide nucleic acid comprises a single polynucleotide. In preferred embodiments, the guide nucleic acid comprises a dual guide nucleic acid (as described in the Guide nucleic acids section below), wherein the targeter nucleic acid and the modulator nucleic acid are separate polynucleotides. In certain embodiments, the guide nucleic acid comprises one or more chemical modifications to one or more nucleotides and/or internucleotide linkages at or near the 5′ end, the 3′ end, and/or both as described in the gNA modifications section below.

In certain embodiments, the guide nucleic acid further comprises a nucleic acid-guided nuclease. The guide nucleic acid can be combined and/or complexed with any suitable nucleic acid-guided nuclease. In certain embodiments, the nucleic acid-guided comprises a Type V CRISPR endonuclease, preferably MAD2, MAD7, ART2, ART11, and/or ART11*, more preferably MAD7.

In certain embodiments, the guide nucleic acid further comprises a nucleic acid-guided nuclease. Any suitable donor template can be combined with the guide nucleic acid. In certain embodiments, the guide nucleic acid comprises a donor template as described in the Donor templates section below. In certain embodiments, the donor template comprises a transgene. In preferred embodiments, the transgene comprises a polynucleotide coding for B2M fusion protein, such as a B2M-HLA-1 subunit fusion protein. In certain embodiments, the HLA-1 subunit comprises HLA-C, HLA-E, or HLA-G, preferably HLA-E or HLA-G. In a preferred embodiment the subunit is HLA-E. In a more preferred embodiment, the subunit is HLA-G. Additionally or alternatively, the cell can comprise a transgene comprising a polynucleotide coding for a CAR or portion thereof. In certain embodiments, the transgene comprises a polynucleotide coding for a dual CAR or portions thereof, e.g., a CAR or portion thereof fusion protein. In certain embodiments, the dual CAR comprises a first CAR or portion thereof and a second CAR or portion thereof, wherein the second CAR or portion thereof is different from the first CAR or portion thereof. In certain embodiments, the CAR or portion thereof comprises a polypeptide that binds at least one of B7H3, BCMA, GPRC5D, CD8, CD8a, CD19, CD20, CD22, CD28, 4-1BB, or CD3zeta. In a preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-124. In a more preferred embodiment, the CAR or portion thereof comprises a polypeptide that binds at least one of B7H3, BCMxA, GPRC5D, CD8, CD8a, CD20, CD22, CD28, 4-1BB, or CD3zeta. In an even more preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

In certain embodiments, the guide nucleic acid, nucleic acid-guided nuclease, and/or donor template can further comprise a cell. The cell can be any suitable cell. In certain embodiments, the cell is a human cell, such as human stem cell or human immune cell, such as an immune cell comprising a neutrophil, cosinophil, basophil, mast cell, monocyte, macrophage, dendritic cell, natural killer cell, or a lymphocyte. In a preferred embodiment, the human immune cell is a T cell. In certain embodiments, the T cell comprises a chimeric antigen receptor (CAR) T cell. In certain embodiments, the CAR T cell expresses a plurality of different CARS, e.g., two different CARs (dual CAR T cell). In certain embodiments, the human cell is a human stem cell comprising a human pluripotent stem cell, multipotent stem cell, embryonic stem cell, induced pluripotent stem cell, hematopoietic stem cell, or a CD34+ cell. In a preferred embodiment, the cell is a hematopoietic stem cell. In a more preferred embodiment, the cell is a CD34+ stem cell. In an even more preferred embodiment, the cell is an induced pluripotent stem cell (iPSC).

f. Compositions Comprising Guide Nucleic Acids Comprising a Spacer Sequence Directed at a Target Nucleotide Sequence in a Gene Coding for a Subunit of an HLA-2 Protein or a Transcription Factor Regulating the Expression of One or More Subunits of an HLA-2 Protein and/or Gene Coding for a Subunit of a TCR Protein

In certain embodiments, provided herein are compositions comprising a first guide nucleic acid comprising a spacer sequence directed at a target nucleotide sequence in a gene coding for a subunit of an HLA-2 protein or a transcription factor regulating the expression of one or more subunits of an HLA-2 protein and a second guide nucleic acid comprising a spacer sequence directed at a target nucleotide sequence in a gene coding for a subunit of a TCR protein. In certain embodiments, the guide nucleic acid comprises a targeter nucleic acid and a modulator nucleic acid, wherein the targeter nucleic acid comprises a targeter nucleic acid comprising a targeter stem sequence and a spacer sequence, and the modulator nucleic acid comprises a modulator stem sequence complementary to the targeter stem sequence, and, optionally, a 5′ sequence. The spacer sequence can be any suitable sequence. In certain embodiments, the spacer sequence comprises any one of SEQ ID NOs: 125-2019. In certain embodiments, the guide nucleic acid comprises a single polynucleotide. In preferred embodiments, the guide nucleic acid comprises a dual guide nucleic acid (as described in the Guide nucleic acids section below), wherein the targeter nucleic acid and the modulator nucleic acid are separate polynucleotides. In certain embodiments, the guide nucleic acid comprises one or more chemical modifications to one or more nucleotides and/or internucleotide linkages at or near the 5′ end, the 3′ end, and/or both as described in the gNA modifications section below.

In certain embodiments, the guide nucleic acid further comprises a nucleic acid-guided nuclease. The guide nucleic acid can be combined and/or complexed with any suitable nucleic acid-guided nuclease. In certain embodiments, the nucleic acid-guided comprises a Type V CRISPR endonuclease, preferably MAD2, MAD7, ART2, ART11, and/or ART11*, more preferably MAD7.

In certain embodiments, the guide nucleic acid further comprises a nucleic acid-guided nuclease. Any suitable donor template can be combined with the guide nucleic acid. In certain embodiments, the guide nucleic acid comprises a donor template as described in the Donor templates section below. In certain embodiments, the donor template comprises a transgene. In preferred embodiments, the transgene comprises a polynucleotide coding for B2M fusion protein, such as a B2M-HLA-1 subunit fusion protein. In certain embodiments, the HLA-1 subunit comprises HLA-C, HLA-E, or HLA-G, preferably HLA-E or HLA-G. In a preferred embodiment the subunit is HLA-E. In a more preferred embodiment, the subunit is HLA-G. Additionally or alternatively, the cell can comprise a transgene comprising a polynucleotide coding for a CAR or portion thereof. In certain embodiments, the transgene comprises a polynucleotide coding for a dual CAR or portions thereof, e.g., a CAR or portion thereof fusion protein. In certain embodiments, the dual CAR comprises a first CAR or portion thereof and a second CAR or portion thereof, wherein the second CAR or portion thereof is different from the first CAR or portion thereof. In certain embodiments, the CAR or portion thereof comprises a polypeptide that binds at least one of B7H3, BCMA, GPRC5D, CD8, CD8a, CD19, CD20, CD22, CD28, 4-1BB, or CD3zeta. In a preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-124. In a more preferred embodiment, the CAR or portion thereof comprises a polypeptide that binds at least one of B7H3, BCMxA, GPRC5D, CD8, CD8a, CD20, CD22, CD28, 4-1BB, or CD3zeta. In an even more preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

In certain embodiments, the guide nucleic acid, nucleic acid-guided nuclease, and/or donor template can further comprise a cell. The cell can be any suitable cell. In certain embodiments, the cell is a human cell, such as human stem cell or human immune cell, such as an immune cell comprising a neutrophil, cosinophil, basophil, mast cell, monocyte, macrophage, dendritic cell, natural killer cell, or a lymphocyte. In a preferred embodiment, the human immune cell is a T cell. In certain embodiments, the T cell comprises a chimeric antigen receptor (CAR) T cell. In certain embodiments, the CAR T cell expresses a plurality of different CARS, e.g., two different CARs (dual CAR T cell). In certain embodiments, the human cell is a human stem cell comprising a human pluripotent stem cell, multipotent stem cell, embryonic stem cell, induced pluripotent stem cell, hematopoietic stem cell, or a CD34+ cell. In a preferred embodiment, the cell is a hematopoietic stem cell. In a more preferred embodiment, the cell is a CD34+ stem cell. In an even more preferred embodiment, the cell is an induced pluripotent stem cell (iPSC).

g. Compositions Comprising Guide Nucleic Acids Comprising a Spacer Sequence Directed at a Target Nucleotide Sequence in a Gene Coding for a Subunit of a TCR Protein

In certain embodiments, provided herein are compositions comprising a first guide nucleic acid comprising a spacer sequence directed at a target nucleotide sequence in a gene coding for a subunit of a TCR protein. In certain embodiments, the guide nucleic acid comprises a targeter nucleic acid and a modulator nucleic acid, wherein the targeter nucleic acid comprises a targeter nucleic acid comprising a targeter stem sequence and a spacer sequence, and the modulator nucleic acid comprises a modulator stem sequence complementary to the targeter stem sequence, and, optionally, a 5′ sequence. The spacer sequence can be any suitable sequence. In certain embodiments, the spacer sequence comprises any one of SEQ ID NOs: 125-2019. In certain embodiments, the guide nucleic acid comprises a single polynucleotide. In preferred embodiments, the guide nucleic acid comprises a dual guide nucleic acid (as described in the Guide nucleic acids section below), wherein the targeter nucleic acid and the modulator nucleic acid are separate polynucleotides. In certain embodiments, the guide nucleic acid comprises one or more chemical modifications to one or more nucleotides and/or internucleotide linkages at or near the 5′ end, the 3′ end, and/or both as described in the gNA modifications section below.

In certain embodiments, the guide nucleic acid further comprises a nucleic acid-guided nuclease. The guide nucleic acid can be combined and/or complexed with any suitable nucleic acid-guided nuclease. In certain embodiments, the nucleic acid-guided comprises a Type V CRISPR endonuclease, preferably MAD2, MAD7, ART2, ART11, and/or ART11*, more preferably MAD7.

In certain embodiments, the guide nucleic acid further comprises a nucleic acid-guided nuclease. Any suitable donor template can be combined with the guide nucleic acid. In certain embodiments, the guide nucleic acid comprises a donor template as described in the Donor templates section below. In certain embodiments, the donor template comprises a transgene. In preferred embodiments, the transgene comprises a polynucleotide coding for B2M fusion protein, such as a B2M-HLA-1 subunit fusion protein. In certain embodiments, the HLA-1 subunit comprises HLA-C, HLA-E, or HLA-G, preferably HLA-E or HLA-G. In a preferred embodiment the subunit is HLA-E. In a more preferred embodiment, the subunit is HLA-G. Additionally or alternatively, the cell can comprise a transgene comprising a polynucleotide coding for a CAR or portion thereof. In certain embodiments, the transgene comprises a polynucleotide coding for a dual CAR or portions thereof, e.g., a CAR or portion thereof fusion protein. In certain embodiments, the dual CAR comprises a first CAR or portion thereof and a second CAR or portion thereof, wherein the second CAR or portion thereof is different from the first CAR or portion thereof. In certain embodiments, the CAR or portion thereof comprises a polypeptide that binds at least one of B7H3, BCMA, GPRC5D, CD8, CD8a, CD19, CD20, CD22, CD28, 4-1BB, or CD3zeta. In a preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-124. In a more preferred embodiment, the CAR or portion thereof comprises a polypeptide that binds at least one of B7H3, BCMxA, GPRC5D, CD8, CD8a, CD20, CD22, CD28, 4-1BB, or CD3zeta. In an even more preferred embodiment, the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

donor template can further comprise a cell. The cell can be any suitable cell. In certain embodiments, the cell is a human cell, such as human stem cell or human immune cell, such as an immune cell comprising a neutrophil, eosinophil, basophil, mast cell, monocyte, macrophage, dendritic cell, natural killer cell, or a lymphocyte. In a preferred embodiment, the human immune cell is a T cell. In certain embodiments, the T cell comprises a chimeric antigen receptor (CAR) T cell. In certain embodiments, the CAR T cell expresses a plurality of different CARS, e.g., two different CARs (dual CAR T cell). In certain embodiments, the human cell is a human stem cell comprising a human pluripotent stem cell, multipotent stem cell, embryonic stem cell, induced pluripotent stem cell, hematopoietic stem cell, or a CD34+ cell. In a preferred embodiment, the cell is a hematopoietic stem cell. In a more preferred embodiment, the cell is a CD34+ stem cell. In an even more preferred embodiment, the cell is an induced pluripotent stem cell (iPSC).

TABLE 2
Spacer sequences

| SEQ ID NO | Name | Sequence |
|---|---|---|
| 125 | Tap1_001 | GATTTCGCTTTCCCCTAAATG |
| 126 | Tap1_002 | GCTTTCCCCTAAATGGCTGAG |
| 127 | Tap1_003 | CCCTAAATGGCTGAGCTTCTC |
| 128 | Tap1_004 | CGAGAGCCCCGCCCTCGTTCC |
| 129 | Tap1_005 | GCTCTTGGAGCCAACCGTTGC |
| 130 | Tap1_006 | AAGCCATTAGCTGCGGCACTG |
| 131 | Tap1_007 | TGCCACAGGGCTGCTGCGGGC |
| 132 | Tap1_008 | CAGAGCCGCCCTGACCGCCGG |
| 133 | Tap1_009 | TCCAGGGGAGATGGCCATTCC |
| 134 | Tap1_010 | CGGGCCGCCTCACTGACTGGA |
| 135 | Tap1_011 | GAGTGAAGGTATCGGCTGAGC |
| 136 | Tap1_012 | AGCCCCCAGACCTGGCTATGG |
| 137 | Tap1_013 | CGGGTTCTTTATAGTGCAGTG |
| 138 | Tap1_014 | TAGTGCAGTGCTGGAGTTCGT |
| 139 | Tap1_015 | GGGCTGTCCTGCGCCAGGAGA |
| 140 | Tap1_016 | AGGAGAAACCTGTCTGGTTCT |
| 141 | Tap1_017 | CAACAGAACCAGACAGGTTTC |
| 142 | Tap1_018 | TGTGGTACCTGGTGCGAGGCC |
| 143 | Tap1_019 | CCACCTTCTTGGGCAGAAGGA |
| 144 | Tap1_020 | CTTCTGCCCAAGAAGGTGGGA |
| 145 | Tap1_021 | CCAGAGATTCCCGCACCTGCA |
| 146 | Tap1_022 | CCTAAACTTCTGGGCTTCGCC |
| 147 | Tap1_023 | CCAACGAGGAGGGCGAAGCCC |
| 148 | Tap1_024 | TTGCAGCTTTTCCCTAAACTT |
| 149 | Tap1_025 | TTTCTTGCAGCTTTTCCCTAA |
| 150 | Tap1_026 | GGGAAAAGCTGCAAGAAATAA |
| 151 | Tap1_027 | AGCAGCATACCTGAAATCTAT |
| 152 | Tap1_028 | TGGTCTCTTTATAGATTTCAG |
| 153 | Tap1_029 | TAGATTTCAGGTATGCTGCTG |
| 154 | Tap1_030 | AGGTATGCTGCTGAAAGTGGG |
| 155 | Tap1_031 | TTCTCTACCAGATGCAGTTCA |
| 156 | Tap1_032 | ACTATTCTTACCTCCCTCTAG |
| 157 | Tap1_033 | TCTGAGGAGCCCACAGCCTTC |
| 158 | Tap1_034 | AGTACCTGGACCGCACCCCTC |
| 159 | Tap1_035 | GGTAGGCAAAGGAGACATCTT |
| 160 | Tap1_036 | CCTACCCAAACCGCCCAGATG |
| 161 | Tap1_037 | ATCTCAGGTGGCTGCAGTGGG |
| 162 | Tap1_038 | TTGAAGACTTCTTCCAAATAC |
| 163 | Tap1_039 | GAAGAAGTCTTCAAGAAAATA |
| 164 | Tap1_040 | CTCCATAGTTGGCTTCTGGGT |
| 165 | Tap1_041 | CTGCAGCAGCTGTGATTTCCT |
| 166 | Tap1_042 | ATCTCTGGACTCCCTCAGGGC |
| 167 | Tap1_043 | CTCTGCAGAGGTAGACGAGGC |
| 168 | Tap1_044 | CGGATCAATGCTCGGGCCAAC |
| 169 | Tap1_045 | CATCCAGGGCACTGGTGGCAT |
| 170 | Tap1_046 | GTACAGGAGCTGCTCCACCTG |
| 171 | Tap1_047 | CTCAGGTGGAGCAGCTCCTGT |
| 172 | Tap1_048 | TGGAAGGAGGCGCTATCCGGG |
| 173 | Tap1_049 | TCCATGAGCTGCTGGTGGGTT |
| 174 | Tap1_050 | ATTCTGGAGCATCTGCAGGAG |
| 175 | TAP2_001 | TCATCTCGTATCCGTTGACAG |
| 176 | TAP2_002 | CTGTGGCTGCTTCAGGGCCCT |
| 177 | TAP2_003 | CTTCCTCAAGGGCTGCCAGGA |
| 178 | TAP2_004 | GCAGCCCCCACAGCCCTCCCA |
| 179 | TAP2_005 | TGGGGACACTGCTGCTCCCGC |
| 180 | TAP2_006 | TTGTTCACCTGGTCCTGCTCC |
| 181 | TAP2_007 | ATGCCTCTTTCAGGTGAGACA |
| 182 | TAP2_008 | AGGTGAGACATTAATCCCTCA |
| 183 | TAP2_009 | ACCCCCATGCCTTTGCCAGTG |
| 184 | TAP2_010 | CCAGTGCCATCTTCTTCATGT |
| 185 | TAP2_011 | TCCTCCCTGCTGCGCCAGGAC |
| 186 | TAP2_012 | TTCCAGGAGACTAAGACAGGT |
| 187 | TAP2_013 | CTCACCTGCTCTGTCCTTCTT |
| 188 | TAP2_014 | AAGGAAGCCAGTTACTCATCA |
| 189 | TAP2_015 | ACCAGGCTTCGCAAGAGCACA |
| 190 | TAP2_016 | AATGCCAATGTGCTCTTGCGA |
| 191 | TAP2_017 | TCTGCTGCACATGCCCTTCAC |
| 192 | TAP2_018 | GGGTTCCCTTACATGCACGCT |
| 193 | TAP2_019 | CTGGCCCTCTTTTCCAGGAAG |
| 194 | TAP2_020 | CAGGAAGTGCTTCGGGAGATC |
| 195 | TAP2_021 | TAGCGACAGACTTCATGCTCC |
| 196 | TAP2_022 | GGGCCGAGGAGCATGAAGTCT |
| 197 | TAP2_023 | ACAACCACTCTGGTATCTTAC |
| 198 | TAP2_024 | TTCTCCTCTTCCAGGTGCTGC |
| 199 | TAP2_025 | CTTTATGATCTACCAGGAGAG |
| 200 | TAP2_026 | TGATCTACCAGGAGAGCGTGG |
| 201 | TAP2_027 | CAGACCCTGGTATACATATAT |
| 202 | TAP2_028 | GCTGTCGGTCCATGTAGGAGA |
| 203 | TAP2_029 | TCCTACATGGACCGACAGCCA |
| 204 | TAP2_030 | ACAACCCCCTGCAGAGTGGTG |
| 205 | TAP2_031 | CATATCCCAATCGCCCTGACA |
| 206 | TAP2_032 | AGGCACCTTGAGCACAGGCCT |
| 207 | TAP2_033 | CTCCCTCTTTCAGGCACCTTG |
| 208 | TAP2_034 | TGGTTTTCTAGGGGCTGACGT |
| 209 | TAP2_035 | TAGGGGCTGACGTTTACCCTA |
| 210 | TAP2_036 | CCCTACGTCCTGGTGAGGTGA |
| 211 | TAP2_037 | ATCCAGCAGCACCTGTCCCCC |
| 212 | TAP2_038 | AGTTGGGCAGGAGCCTGTGCT |
| 213 | TAP2_039 | CTGGATGAAGTCATCTGCGTG |
| 214 | TAP2_040 | TAGAAGATACCTGTGTATATT |
| 215 | TAP2_041 | TCTGATTTCCTCAGATGTAGG |
| 216 | TAP2_042 | CTCAGATGTAGGGGAGAAGGG |
| 217 | TAP2_043 | TGTCCCGCAGCCAGCTGGCTT |
| 218 | TAPBP_001 | CGCTCGCATCCTCCACGAACC |
| 219 | TAPBP_002 | GCAGAGGCGGGGAGAGGCACG |
| 220 | TAPBP_003 | CCTACATGCCCCCCACCTCCG |
| 221 | TAPBP_004 | GGCTAGAGTGGCGACGCCAGC |
| 222 | TAPBP_005 | CTGCTTGGGATGATGATGAGC |
| 223 | TAPBP_006 | CGGTCCATGGGCCCCATGGCT |
| 224 | TAPBP_007 | AGGAGGGCACCTATCTGGCCA |
| 225 | TAPBP_008 | GGGGGTTCTGGGGAAAGAGGA |
| 226 | TAPBP_009 | CCTATGCTCATTTCGTCCTCT |
| 227 | TAPBP_010 | GTCCTCTTTCCCCAGAACCCC |
| 228 | TAPBP_011 | CCCAGAACCCCCCAAAGTGTC |
| 229 | TAPBP_012 | AGGGCCCTCCCTTGAGGACAG |
| 230 | TAPBP_013 | CTGTCTGCCTTTCTTCTGCTT |
| 231 | TAPBP_014 | TTCTGCTTGGGCTCTTCAAGG |
| 232 | TAPBP_015 | AATCCTTGCAGGTGGACAGGT |
| 233 | TAPBP_016 | CCCACAGCTGTCTACCTGTCC |
| 234 | PSMB9_001 | ACGGGGGCGTTGTGATGGGTT |
| 235 | PSMB9_002 | CTCACCCTGCAGACACTCGGG |
| 236 | PSMB9_003 | ACAAGCTGTCCCCGCTGCACG |
| 237 | PSMB9_004 | TTCCTAATATTTCCCTCAGGA |
| 238 | PSMB9_005 | CCTCAGGATAGAACTGGAGGA |
| 239 | PSMB9_006 | CAGCAGCCAAAACAAGTGGAG |
| 240 | PSMB9_007 | TCACCACATTTGCAGCAGCCA |
| 241 | PSMB9_008 | TAGCTGATATTTCTCACCACA |
| 242 | PSMB9_009 | GCTGCTGCAAATGTGGTGAGA |
| 243 | PSMB9_010 | GGAGAAACTCACCTGACCTCC |
| 244 | PSMB9_011 | ACCTGAGGATCCCTTTCCCAG |
| 245 | PSMB9_012 | CCAGGTATATGGAACCCTGGG |
| 246 | PSMB9_013 | CCATTGGTGGCTCCGGCAGCA |
| 247 | PSMB9_014 | TCTATGGTTATGTGGATGCAG |
| 248 | PSMB9_015 | GCAGTTCATTGCCCAAGATGA |
| 249 | PSMB8_001 | TCTATGCGATCTCCAGAGCTC |
| 250 | PSMB8_002 | CCCCGGGGAATGCAGGTCGGG |
| 251 | PSMB8_003 | TCAACCTCTTTTCTCTTATCA |
| 252 | PSMB8_004 | TCTTATCAGCCCACAGAATTC |
| 253 | PSMB8_005 | TCCGTCCCCACCCAGGGACTG |
| 254 | PSMB8_006 | CTCACTCCACCTTGTCCTCAC |
| 255 | PSMB8_007 | GCAGATAGTACAGCCTGGGTG |
| 256 | PSMB8_008 | AGTGTCGGCAGCCTCCAAGCT |
| 257 | PSMB8_009 | AGCCTGAAATCTTTCATCTTA |
| 258 | PSMB8_010 | ATCTTATAGGGTCCTGGACTC |
| 259 | PSMB8_011 | CTGAGAGCCGAGTCCCATGTT |
| 260 | PSMB8_012 | TCATTTGTCCACAGTGTACCA |
| 261 | PSMB8_013 | ACCCAACCATCTTCCTTCATG |
| 262 | PSMB8_014 | TCCACAGTGTACCACATGAAG |
| 263 | PSMB8_015 | TACTTTCACCCAACCATCTTC |
| 264 | MCL1_001 | TCAGCCAGGCGGCGGCGGCGA |
| 265 | MCL1_002 | AGGCCAAACATTGCCAGTCGC |
| 266 | MCL1_003 | TTTTGAGGCCAAACATTGCCA |
| 267 | MCL1_004 | GCCTCAAAAGAAACGCGGTAA |
| 268 | MCL1_005 | GCTACGGAGAAGGAGGCCTCG |
| 269 | MCL1_006 | TTCGCGCCCACCCGCCGCGCG |
| 270 | MCL1_007 | TGTCCTTGGCGCCGGTGGCCT |
| 271 | MCL1_008 | ATGTCCAGTTTCCGAAGCATG |
| 272 | MCL1_009 | TTTCTCAGGCATGCTTCGGAA |
| 273 | MCL1_010 | TCAGGCATGCTTCGGAAACTG |
| 274 | MCL1_011 | ACATCGTCTTCGTTTTTGATG |
| 275 | MCL1_012 | TTACGCCGTCGCTGAAAACAT |
| 276 | MCL1_013 | AGCGACGGCGTAACAAACTGG |
| 277 | MCL1_014 | GCCACAAAGGCACCAAAAGAA |
| 278 | MCL1_015 | TGGTCTTCAAGTGTTTAGCCA |
| 279 | MCL1_016 | TTTTGGTGCCTTTGTGGCTAA |
| 280 | MCL1_017 | GTGCCTTTGTGGCTAAACACT |
| 281 | MCL1_018 | TTGGTTTATGGTCTTCAAGTG |
| 282 | MCL1_019 | TGGCTAAACACTTGAAGACCA |
| 283 | MCL1_020 | TGCTAATGGTTCGATGCAGCT |
| 284 | MCL1_021 | TCCTTACGAGAACGTCTGTGA |
| 285 | MCL1_022 | ACTAGCCAGTCCCGTTTTGTC |
| 286 | MCL1_023 | TTTAACTAGCCAGTCCCGTTT |
| 287 | MCL1_024 | ATCCTTAAGGCAAACTTACCC |
| 288 | MCL1_025 | TTTTTTGTTTTCTAGGATGGG |
| 289 | MCL1_026 | TTTTCTAGGATGGGTTTGTGG |
| 290 | MCL1_027 | TAGGATGGGTTTGTGGAGTTC |
| 291 | MCL1_028 | TGGAGTTCTTCCATGTAGAGG |
| 292 | MCL1_029 | CAGGTGTTGCTGGAGTAGGAG |
| 293 | MCL1_030 | GCATATCTAATAAGATAGCCT |
| 294 | PSMB5_001 | TGCCCACACTAGACATGGCGC |
| 295 | PSMB5_002 | GGACTTGGGGGTCGTGCAGAT |
| 296 | PSMB5_003 | GATTCCTGGCTCTTCTGGGAC |
| 297 | PSMB5_004 | TGTTTTCCTCTGATCTTAACA |
| 298 | PSMB5_005 | CTCTGATCTTAACAGTTCCGC |
| 299 | PSMB5_006 | GAAGCTCATAGATTCGACATT |
| 300 | PSMB5_007 | GAGGCAGCTGCTACAGAGATG |
| 301 | PSMB5_008 | TACTGATACACCATGTTGGCA |
| 302 | PSMB5_009 | CTGCTAACCTCATCTCCCTTT |
| 303 | PSMB5_010 | CAGGCCTCTACTACGTGGACA |
| 304 | PSMB5_011 | AGGGGCCACCTTCTCTGTAGG |
| 305 | PSMB5_012 | AGGGGGTAGAGCCACTATACT |
| 306 | CALR_001 | GATTCGATCCAGCGGGAAGTC |
| 307 | CALR_002 | CCAAAATCTGACTTGTGTTTG |
| 308 | CALR_003 | GCAAATTCGTTCTCAGTTCCG |
| 309 | CALR_004 | TCCTCGTCACCGTAGAACTTG |
| 310 | CALR_005 | TCTTTCTCCTCGTCACCGTAG |
| 311 | CALR_006 | CAGACAAGCCAGGATGCACGC |
| 312 | CALR_007 | TTGCTGAAAGGCTCGAAACTG |
| 313 | CALR_008 | TGCTCTGTCGGCCAGTTTCGA |
| 314 | CALR_009 | GAGCCTTTCAGCAACAAAGGC |
| 315 | CALR_010 | AGCAACAAAGGCCAGACGCTG |
| 316 | CALR_011 | ACCGTGAACTGCACCACCAGC |
| 317 | CALR_012 | CTAATAGTTTGGACCAGACAG |
| 318 | CALR_013 | GACCAGACAGACATGCACGGA |
| 319 | CALR_014 | CCACCACCCCCAGGCACACCT |
| 320 | CALR_015 | CACACCTGTACACACTGATTG |
| 321 | CALR_016 | TCTTCTTGGGTGGCAGGAAGT |
| 322 | CALR_017 | AAGCATCAGGATCCTTTATCT |
| 323 | CALR_018 | CTTCTCCCTTCTGCAGGGTGA |
| 324 | CALR_019 | TGGGTGGATCCAAGTGCCCTT |
| 325 | CALR_020 | GCGTGCTGGGCCTGGACCTCT |
| 326 | CALR_021 | CTCCAAGTCTCACCTGCCAGA |
| 327 | CALR_022 | ACAACTTCCTCATCACCAACG |
| 328 | CALR_023 | TTACGCCCCACGTCTCGTTGC |
| 329 | CALR_024 | GCAACGAGACGTGGGGCGTAA |
| 330 | CALR_025 | TCCTTCATTTGTTTCTCTGCT |
| 331 | CALR_026 | ttgtcttcttcctcctccttA |
| 332 | CALR_027 | cgtttcttgtcttcttcctcc |
| 333 | CALR_028 | tcctcatcatcctccttgtcc |
| 334 | APLNR_001 | ACAACTACTATGGGGCAGACA |
| 335 | APLNR_002 | CAGTCTGTGTACTCACACTCA |
| 336 | APLNR_003 | GGAGCAGCCGGGAGAAGAGGC |
| 337 | APLNR_004 | GGACCTTCTTCTGCAAGCTCA |
| 338 | APLNR_005 | GGTGCTGGCCGCCCTCCTGGC |
| 339 | APLNR_006 | TGGTGCCCTTCACCATCATGC |
| 340 | APLNR_007 | GGCGATGAAGAAGTAACAGGT |
| 341 | APLNR_008 | CCCTGTGCTGGATGCCCTACC |
| 342 | APLNR_009 | ACCTCTTCCTCATGAACATCT |
| 343 | APLNR_010 | GACCCCCGCTTCCGCCAGGCC |
| 344 | APLNR_011 | TCGTGCATCTGTTCTCCACCC |
| 345 | BBS1_001 | CCCCACTTCCAGCAATGAGGC |
| 346 | BBS1_002 | GCCCTTTTGTTTTCCAGCGCT |
| 347 | BBS1_003 | TTTTCCAGCGCTGGCAGATTT |
| 348 | BBS1_004 | CAGCGCTGGCAGATTTACATG |
| 349 | BBS1_005 | CATGGGGATGGGGAATACAAG |
| 350 | BBS1_006 | AGCACCTTCAGGCGGGGCTGC |
| 351 | BBS1_007 | GGTCATCACCAGTGGTCCTTT |
| 352 | BBS1_008 | GAGGCAATTGGGGCAGGCTGA |
| 353 | BBS1_009 | GCCTGGTTCCAAAGGTCTTGT |
| 354 | BBS1_010 | CCTCTTTGGCCTGGTTCCAAA |
| 355 | BBS1_011 | TTTACCTCTTTGGCCTGGTTC |
| 356 | BBS1_012 | GAACCAGGCCAAAGAGGTAAA |
| 357 | BBS1_013 | CTTGCAGGGAGACGGCAGAGG |
| 358 | BBS1_014 | TCCATCCAGTCACTCAGGTAA |
| 359 | BBS1_015 | ACTTAGCTCCAGCTGCAGAAA |
| 360 | BBS1_016 | CAAATGCCTCCATTTCACTTA |
| 361 | BBS1_017 | TGCAGCTGGAGCTAAGTGAAA |
| 362 | BBS1_018 | TAAACCAACACAAGTCCAACT |
| 363 | BBS1_019 | CGCTTCTTGTTTGCAGATGAG |
| 364 | BBS1_020 | CAGATGAGCCTTCCCAGCGTC |
| 365 | BBS1_021 | TGGCCAGTTTGATGTTGAGTT |
| 366 | BBS1_022 | CATTGCGGCAGGCCGCGGCAA |
| 367 | BBS1_023 | ATGTTGAGTTCCGGCTTGCCG |
| 368 | BBS1_024 | TTCCACAGAGACTCCAAGCAC |
| 369 | BBS1_025 | TCGTGACAAGGCCCTGCTCAA |
| 370 | BBS1_026 | CTTTGGCCGGTACGGGCGGGA |
| 371 | BBS1_027 | GCCGGTACGGGCGGGAGGACA |
| 372 | BBS1_028 | CACTGTCCACTTCCCTAGGTG |
| 373 | BBS1_029 | TAGAGGGAGGAAGTGAGGTGG |
| 374 | BBS1_030 | ATGGCCTGGGCTGGTGGGGGA |
| 375 | BBS1_031 | GGGGCACATTGAGTTTCATGG |
| 376 | BBS1_032 | CGTGGATCAGACACTGCGAGA |
| 377 | BBS1_033 | TCCACCCACCCTCTCCATAGG |
| 378 | BBS1_034 | AGCTCACACTTCACCTGCAGA |
| 379 | BBS1_035 | TCCCCAAACTTAGGTACCCTT |
| 380 | BBS1_036 | TGGAGAGTCTCAGTAACAAGG |
| 381 | BBS1_037 | GCCTTCTCGAAGCACCAGCAC |
| 382 | BBS1_038 | ACAGCAGCTCAGGTCTCAGGC |
| 383 | RFX5_001 | TTCTGCACGGCCTTGCTGTGG |
| 384 | RFX5_002 | TCCTCTTCCCCACAGCAAGGC |
| 385 | RFX5_003 | TCCTGCCTCTGTTCTCTCCTA |
| 386 | RFX5_004 | TGTACATCTTGCTGAGGTAGG |
| 387 | RFX5_005 | TGACAATGACAAGCTGTATCT |
| 388 | RFX5_006 | TCTCCAGTGGTGGGTCCTGAG |
| 389 | RFX5_007 | TTTCTGTAGCTCAGAGCCAAG |
| 390 | RFX5_008 | TGTAGCTCAGAGCCAAGTACA |
| 391 | RFX5_009 | GCAGACAGGTGTCAGTGTGCT |
| 392 | RFX5_010 | CTTTGGCAGACAGGTGTCAGT |
| 393 | RFX5_011 | ATGTCAGGGAAGATCTCTCTG |
| 394 | RFX5_012 | GCAAGATCATCAGAGAGATCT |
| 395 | RFX5_013 | ACTTGCATCAGATATTGCTAC |
| 396 | RFX5_014 | GGTCAAGTCCAGGCAGGGGTG |
| 397 | RFX5_015 | GTACTTACACTCTCAGAACCC |
| 398 | RFX5_016 | AGGATCCGCTCTGCCCAGTCA |
| 399 | RFX5_017 | GTACCTCTGCAGAAGAGGACG |
| 400 | RFX5_018 | GATGACCGTTCCCGAGGTGCA |
| 401 | RFX5_019 | GTTTAGATGACCGTTCCCGAG |
| 402 | RFX5_020 | GAGAACCCAGAGGGTGGAGCC |
| 403 | RFX5_021 | CTTTTTAGCCTCCTAAGGATC |
| 404 | RFX5_022 | GCCTCCTAAGGATCTGGAAGC |
| 405 | RFX5_023 | AGGGCACCTGAAGAAAGCCTG |
| 406 | RFX5_024 | TTCAGGTGCCCTGAAAGTGGC |
| 407 | RFX5_025 | CCTGGACCTGGACCTGGGCCT |
| 408 | RFX5_026 | GCTGGTGGAGCCTGCCCACTG |
| 409 | RFX5_027 | CTGCTTTAGCTGGTGGAGCCT |
| 410 | RFX5_028 | GCATCACTTGCTGTATCCTCT |
| 411 | RFX5_029 | CTTTTGGCATCACTTGCTGTA |
| 412 | RFX5_030 | GAGGGCGCCCCCGTTTCCTTT |
| 413 | RFX5_031 | CCCACTTCCACCTGACTTTTT |
| 414 | RFX5_032 | AGCCTCTCCCATTGGCCCTGG |
| 415 | RFX5_033 | GAAACAGTACCATCTCCCTGA |
| 416 | RFX5_034 | GTATGCTGGGAACCGGGGCCC |
| 417 | RFX5_035 | CAAAGGAGGAAGGGGCCCCGG |
| 418 | RFX5_036 | TCTTCTGCTTCTTTGGTATGC |
| 419 | RFX5_037 | AGGGGACCAAGGGAATTTTAT |
| 420 | RFX5_038 | GCTTCTGCTGCCCTTGATGAC |
| 421 | RFX5_039 | CCAAAGGAAAAGCCTCCTTTT |
| 422 | RFX5_040 | CTTTGGCAAAGGGAGAGGTAG |
| 423 | RFX5_041 | TTACCCTGTGGTGCAGTGTCT |
| 424 | RFX5_042 | GCAAAGGGAGAGGTAGACACT |
| 425 | RFX5_043 | AGTCTTTATTACCCTGTGGTG |
| 426 | RFX5_044 | AAGCACATGCTCCTTTAAGTC |
| 427 | RFX5_045 | TGCTCCTGGGATAAGGAACTT |
| 428 | RFX5_046 | GGTCTTTATGCTCCTGGGATA |
| 429 | RFXAP_001 | GAGGATCTAGAGGACGAGGAG |
| 430 | RFXAP_002 | CGCTGCTTGGCCACCTGGCTC |
| 431 | RFXAP_003 | TTGCACATCCACGGTTTGCGC |
| 432 | RFXAP_004 | TACTTGTCCTTGTACATCTTG |
| 433 | RFXAP_005 | CCGCGCTGCCAGTCGAGGCAG |
| 434 | RFXAP_006 | ACGTTTCCCGCGCTGCCAGTC |
| 435 | RFXAP_007 | CATTTTTATCATTTATCCCAG |
| 436 | RFXAP_008 | TCATTTATCCCAGGAAAGTGC |
| 437 | RFXAP_009 | ACAATGGAGAGTATGTTATCT |
| 438 | RFXAP_010 | TCCCAGGAAAGTGCAGATAAC |
| 439 | RFXAP_011 | TTTAACAATGGAGAGTATGTT |
| 440 | RFXAP_012 | GGGATCGTCCTGCAAGACCTA |
| 441 | RFXAP_013 | ACACTTGTTCTAAAAGAGTAG |
| 442 | RFXAP_014 | ATTTAACACTTGTTCTAAAAG |
| 443 | RFXAP_015 | CCAGTCTTTTTTGATTTAACA |
| 444 | RFXAP_016 | GAACAAGTGTTAAATCAAAAA |
| 445 | RFXAP_017 | CAAACAGATATTTACCAGTCT |
| 446 | RFXAP_018 | TTTTCTTTCTAAGTCGTTACT |
| 447 | RFXAP_019 | TTTCTAAGTCGTTACTAAGAA |
| 448 | RFXAP_020 | TAAGTCGTTACTAAGAAGTCC |
| 449 | RFXAP_021 | TGTAAAAATTGCACTACTTCT |
| 450 | RFXAP_022 | ATAGCTGTTGCTGTTTCTGTA |
| 451 | RFXAP_023 | CAGAAACAGCAACAGCTATTA |
| 452 | RFXAP_024 | CTCCAAAACTTGCTGATTTAA |
| 453 | RFXAP_025 | GAGCAAAGACAACAGCAGTTT |
| 454 | RFXAP_026 | CAGGAACATCAATGTGAGGGA |
| 455 | RFXANK_001 | CCCATGGAGCTTACCCAGCCT |
| 456 | RFXANK_002 | CCTGCACCCCTGAGCCTGTGA |
| 457 | RFXANK_003 | CCAGCAGGCAGCTCCCTGAAG |
| 458 | RFXANK_004 | CGCAAATGCTCCTTCAGCTGG |
| 459 | RFXANK_005 | GAGAGATTGAGACCGTTCGCT |
| 460 | RFXANK_006 | CCAGGATGTGGGGGTCGGCAC |
| 461 | RFXANK_007 | TCCTGCCCCTACCCACGACAG |
| 462 | RFXANK_008 | ACGTGGTTCCCGCGCACAGCG |
| 463 | RFXANK_009 | CAGCCCGAGGCGCTGACCTCA |
| 464 | RFXANK_010 | CGGTATCCCAGGGCCACGGCA |
| 465 | RFXANK_011 | CCTGCCCCATCTCAGTGCAAC |
| 466 | CD58_001 | TTGGGAAAAACAGCTGATGAA |
| 467 | CD58_002 | TCTCCTAGGTTTCATCAGCTG |
| 468 | CD58_003 | ATCAGCTGTTTTTCCCAACAA |
| 469 | CD58_004 | CCAACAAATATATGGTGTTGT |
| 470 | CD58_005 | AAGGCACATTGCTTGGTACAT |
| 471 | CD58_006 | CATGTACCAAGCAATGTGCCT |
| 472 | CD58_007 | CATAGGACCTCTTTTAAAGGC |
| 473 | CD58_008 | TTTTTTCCATAGGACCTCTTT |
| 474 | CD58_009 | TCCTTTTGTTTTTTCCATAGG |
| 475 | CD58_010 | AAAGAGGTCCTATGGAAAAAA |
| 476 | CD58_011 | CAGTTCTGCAACTTTATCCTT |
| 477 | CD58_012 | AAAGATGAGAAAGCTCTGAAT |
| 478 | CD58_013 | TCATCTTTTAAAAATAGGGTT |
| 479 | CD58_014 | AAAATAGGGTTTATTTAGACA |
| 480 | CD58_015 | TTTAGACACTGTGTCAGGTAG |
| 481 | CD58_016 | GACACTGTGTCAGGTAGCCTC |
| 482 | CD58_017 | ATACTCATCTTCATCTGATGA |
| 483 | CD58_018 | GCGATTCCATTTCATACTCAT |
| 484 | CD58_019 | CAGAGTCTCTTCCATCTCCCA |
| 485 | CD58_020 | CATTGCTCCATAGGACAATCC |
| 486 | CD58_021 | CATCTTAAAATATATACTGGT |
| 487 | CD58_022 | TGGAAGATCATTTTCCATCTT |
| 488 | CD58_023 | AGATGGAAAATGATCTTCCAC |
| 489 | CD58_024 | ATACAACATCATCAATCATTT |
| 490 | CD58_025 | CTCACCGCTGCTTGGGATACA |
| 491 | CD58_026 | GTTATTTACTCACCGCTGCTT |
| 492 | CD58_027 | ACAACCTGTATCCCAAGCAGC |
| 493 | CD58_028 | TAGGTCATTCAAGACACAGAT |
| 494 | CD58_029 | AAAAGCATACATACCATTCAT |
| 495 | CD58_030 | TTTTAAAAAGCATACATACCA |
| 496 | CD58_031 | TGTCACATTTCAGAATACCTA |
| 497 | CD58_032 | TTCATTTTTAGGTATTCTGAA |
| 498 | CD58_033 | GGTATTCTGAAATGTGACAGA |
| 499 | COL17A1_001 | TTTTTCTTGGTTACATCCATA |
| 500 | COL17A1_002 | CTGCAGGTGGCTATGGTATGG |
| 501 | COL17A1_003 | TTTGTCTTTTTCTAGTTGTCA |
| 502 | COL17A1_004 | TCTTTTTCTAGTTGTCACTGA |
| 503 | COL17A1_005 | TAGTTGTCACTGAAACAGTAA |
| 504 | COL17A1_006 | GCATAGCCATTGCTGGTCCCG |
| 505 | COL17A1_007 | TTCCTGGCAGAAGGCGGGACC |
| 506 | COL17A1_008 | TCCAGCCGGCTCCCTCCACCA |
| 507 | COL17A1_009 | TTTCTCCAGCCGGCTCCCTCC |
| 508 | COL17A1_010 | TGTAGCCGCTGCTGCCATGAG |
| 509 | COL17A1_011 | CCTCTTGCAGCTGGAAGCACA |
| 510 | COL17A1_012 | AAAGGTTGAGCCTGGGGAGTT |
| 511 | COL17A1_013 | CTTTCAAAGGTTGAGCCTGGG |
| 512 | COL17A1_014 | AAAGGAAAACTCACGTTACCC |
| 513 | COL17A1_015 | ATCCCCTCTCCAGGGAGCTCC |
| 514 | COL17A1_016 | TTTGTTTCTCAGCATCTTCTT |
| 515 | COL17A1_017 | ACTCCGTCCTCTGGTTGAAGA |
| 516 | COL17A1_018 | TTTCTCAGCATCTTCTTCAAC |
| 517 | COL17A1_019 | TCAGCATCTTCTTCAACCAGA |
| 518 | COL17A1_020 | ACAGGGACAGAATTGGATGAT |
| 519 | COL17A1_021 | CTCAAGGGGAGTCGATCGGCA |
| 520 | COL17A1_022 | TTGGGGATGGGGAGTGTGTTG |
| 521 | COL17A1_023 | GTCTCCACAGTGCCTTTCTTG |
| 522 | COL17A1_024 | CAGTGTCAGGCACCTACGATG |
| 523 | COL17A1_025 | CACCCTGGACTCAGCACATCC |
| 524 | COL17A1_026 | ACAGTGTTTGGCATGCAGAAC |
| 525 | COL17A1_027 | GCATGCAGAACAATCTGGCCC |
| 526 | COL17A1_028 | TTGCAGCATATGGGGTGAAGA |
| 527 | COL17A1_029 | TTTCTCCCCAGCCTGCACCAC |
| 528 | COL17A1_030 | TCCCCAGCCTGCACCACAAGT |
| 529 | COL17A1_031 | TCTAGGATCAGGAACTTGCAG |
| 530 | COL17A1_032 | CACAAGGACTGCAAGTTCCTG |
| 531 | COL17A1_033 | GGGTGTCTTCTGAAAAAGAAG |
| 532 | COL17A1_034 | TTTTTTTAGGGTGTCTTCTGA |
| 533 | COL17A1_035 | AGAAGACACCCTAAAAAAAGA |
| 534 | COL17A1_036 | GGCCTGAGTCAGCATTGTAGG |
| 535 | COL17A1_037 | TTCTTACCATTAGCTTCGGCT |
| 536 | COL17A1_038 | TGGACACAGTCTTCAGGTCTC |
| 537 | COL17A1_039 | TCCTTTCAGGAGACCTGAAGA |
| 538 | COL17A1_040 | AGGAGACCTGAAGACTGTGTC |
| 539 | COL17A1_041 | CCTGTCTCTTTCACAGATATC |
| 540 | COL17A1_042 | ACAGATATCCACAGCTACGGC |
| 541 | COL17A1_043 | CCACGTACCCAGAGCAATGAG |
| 542 | COL17A1_044 | TTGCAGCGGAGGAGGTGAGGA |
| 543 | COL17A1_045 | TATTCTATCCATGCTGTCCCC |
| 544 | COL17A1_046 | TCCAGGTCTGCTCCCGCCGCG |
| 545 | COL17A1_047 | CTGTTCCATCATTAGCTTCTT |
| 546 | COL17A1_048 | CTTTTTCTTGCAGGAAATCTC |
| 547 | COL17A1_049 | GGGCCAGGGCTTCCTCGGAGA |
| 548 | COL17A1_050 | TTGCAGGAAATCTCCGAGGAA |
| 549 | COL17A1_051 | ATATCTTTCTGGTTTCAGGTG |
| 550 | COL17A1_052 | GGGCCTGGACTTCCCATGTCA |
| 551 | COL17A1_053 | TGGTTTCAGGTGACATGGGAA |
| 552 | COL17A1_054 | AGGTGACATGGGAAGTCCAGG |
| 553 | COL17A1_055 | CCTTTGTTCCTGCAGGAGATC |
| 554 | COL17A1_056 | TTCCTGCAGGAGATCGAGGGT |
| 555 | COL17A1_057 | GTCCTTGTGGACCTGGGTGGC |
| 556 | COL17A1_058 | ACCCTTTGGTCCTTGTGGACC |
| 557 | COL17A1_059 | TTACCCACGCTGCCTTTTTGA |
| 558 | COL17A1_060 | GGAGATCCTGGCATGGAAGGC |
| 559 | COL17A1_061 | TCTCCAGATCCAGGAGGCCCT |
| 560 | COL17A1_062 | CCCTTTCTCTCCAGATCCAGG |
| 561 | COL17A1_063 | TCCTCAGGGGCTGCTGGTGAA |
| 562 | COL17A1_064 | GGACCCACAGAACCTGGGACA |
| 563 | COL17A1_065 | CAAGAAGCAGCAAACTGACCT |
| 564 | COL17A1_066 | TTCTGCCGGGCAGGTCCTGTA |
| 565 | COL17A1_067 | ACACCAGGAAGTCCTACTTCA |
| 566 | COL17A1_068 | CTTTTTAGGTGACAAAGGACC |
| 567 | COL17A1_069 | GGTCCTGGTGGTCCCATTGGT |
| 568 | COL17A1_070 | GGTGACAAAGGACCAATGGGA |
| 569 | COL17A1_071 | CTTTAGGTGACCAGGGTGAGA |
| 570 | COL17A1_072 | GGTGACCAGGGTGAGAAAGGA |
| 571 | COL17A1_073 | TCCTTTGCAGGCGAGCCTGGC |
| 572 | COL17A1_074 | CAGGCGAGCCTGGCATGAGAG |
| 573 | COL17A1_075 | GCCCCGGGCTCACCAACAGCA |
| 574 | COL17A1_076 | CCTGGTGCTGTTGGTGAGCCC |
| 575 | COL17A1_077 | GAACACTTACCCATTGCTCCT |
| 576 | COL17A1_078 | CCAGGTCCTGCTGGCCCAGAC |
| 577 | COL17A1_079 | CTGGGTCTCCAGAAGGTCCTG |
| 578 | COL17A1_080 | TGCAGGTCTCACAGGACCCCA |
| 579 | COL17A1_081 | TTCCTGGTCGGCCAGGGGTAC |
| 580 | COL17A1_082 | GAAATTCACTTACCTTTTATT |
| 581 | COL17A1_083 | CTCTCTTCCTAGGTGAACCAG |
| 582 | COL17A1_084 | AGAGGGGTCATCGATGCTCAC |
| 583 | COL17A1_085 | TTCCTCAACCCCGTTTCCAGG |
| 584 | COL17A1_086 | CAGGCCCTGCCGGCCCAGCTG |
| 585 | COL17A1_087 | TATTTTCTTCTCTCTATAGAA |
| 586 | COL17A1_088 | TTCTCTCTATAGAAGTTCTTA |
| 587 | COL17A1_089 | CAAGGTCCCCCAGGCCCACCC |
| 588 | COL17A1_090 | CTAGGGGAGGGTTTGccaggc |
| 589 | COL17A1_091 | ccaggcccaccaggcccacca |
| 590 | COL17A1_092 | CTTCCTCTGCAGAAACCTTCC |
| 591 | COL17A1_093 | CCTCAGGTCCCCCAGGCCCCA |
| 592 | COL17A1_094 | ATGCCGGCTCTACTGTACCTT |
| 593 | COL17A1_095 | GGACTCAACCTTCAGGGACCA |
| 594 | COL17A1_096 | GGTCCCTGGGGGCCAGGTGGG |
| 595 | COL17A1_097 | TCACCTTTGGGTCCCTGGGGG |
| 596 | COL17A1_098 | GAttccaggtgatccaggtgt |
| 597 | COL17A1_099 | AGTTCTTACCTTCAGAAGGAC |
| 598 | COL17A1_100 | GTCACTTTCAGTTCTTACCTT |
| 599 | COL17A1_101 | TCTTTGCTGCAGGGGGATCAT |
| 600 | COL17A1_102 | CTGCAGGGGGATCATCAAGTA |
| 601 | COL17A1_103 | CTTTGTTCCTTGGTCGGCAGG |
| 602 | COL17A1_104 | TTCCTTGGTCGGCAGGTGACA |
| 603 | COL17A1_105 | GACTACTCAGAGCTGGCAAGC |
| 604 | COL17A1_106 | TTCCCGACAGCTTCGGGGTAC |
| 605 | COL17A1_107 | GACTATGCAGAGCTGAGTAGT |
| 606 | COL17A1_108 | TTTCTCTTCCTTCTGCCCAGC |
| 607 | COL17A1_109 | TCTTCCTTCTGCCCAGCTGCC |
| 608 | COL17A1_110 | AGCTGCATAGGTTGCCAGGGC |
| 609 | COL17A1_111 | GTGAAGCTGCAGGAGACAGGG |
| 610 | COL17A1_112 | CTGGAGATCTGGATTACAATG |
| 611 | COL17A1_113 | CAGGTCAGGGCCTACTGCAAG |
| 612 | COL17A1_114 | GAAGAAGTCCATGAGGTCCGC |
| 613 | COL17A1_115 | CTTGCTTTTGCAGCTTATGGA |
| 614 | COL17A1_116 | CCCAGGGGGTCCTTGAATGGC |
| 615 | COL17A1_117 | CAGCTTATGGAGCCATTCAAG |
| 616 | COL17A1_118 | GGTCCTGGAGTGCCCATCTCT |
| 617 | COL17A1_119 | CTTCCAGGTGACAGGGGCCCT |
| 618 | COL17A1_120 | TCCCTTGTGTCCTCGAGGGCC |
| 619 | COL17A1_121 | TCTCCTTTTTCTCCCTTGTGT |
| 620 | COL17A1_122 | AGGTGACCAAGTCTATGCTGG |
| 621 | DEFB134_001 | CCTGCCAGCACTGGATCCCAA |
| 622 | DEFB134_002 | TCTTTCTTTTCCTTTGGGATC |
| 623 | DEFB134_003 | TTTTCCTTTGGGATCCAGTGC |
| 624 | DEFB134_004 | CTTTGGGATCCAGTGCTGGCA |
| 625 | DEFB134_005 | GGATCCAGTGCTGGCAGGTAA |
| 626 | DEFB134_006 | TGATGATAATGAATTTATACC |
| 627 | DEFB134_007 | CTTCCAGGTATAAATTCATTA |
| 628 | DEFB134_008 | TTGTGCATTTCTGATGATAAT |
| 629 | DEFB134_009 | TAGCATTTCTTGTGCATTTCT |
| 630 | DEFB134_010 | ACTCTCATAGCATTCAAGTCT |
| 631 | DEFB134_011 | ACACAGCACTCCAGCTGAAAC |
| 632 | DEFB134_012 | CTTTGACACAGCACTCCAGCT |
| 633 | DEFB134_013 | AGCTGGAGTGCTGTGTCAAAG |
| 634 | DEFB134_014 | TTATGTCAGGGTGCAGGATTT |
| 635 | MLANA_001 | AACTTACTCTTCAGCCGTGGT |
| 636 | MLANA_002 | TCTATCTCTTGGGCCAGGGCC |
| 637 | MLANA_003 | GTCTTCTACAATACCAACAGC |
| 638 | MLANA_004 | CCAACCATCAAGGCTCTGTAT |
| 639 | MLANA_005 | AGCAGTGGGAACTTTACCAAC |
| 640 | MLANA_006 | TCCTGAAATGTAAATTGATAA |
| 641 | MLANA_007 | TCAATTTACATTTCAGGATAA |
| 642 | MLANA_008 | CATTTCAGGATAAAAGTCTTC |
| 643 | MLANA_009 | AGGATAAAAGTCTTCATGTTG |
| 644 | MLANA_010 | CTGTCCCGATGATCAAACCCT |
| 645 | MLANA_011 | TCTTGAAGAGACACTTTGCTG |
| 646 | MLANA_012 | ATCATCGGGACAGCAAAGTGT |
| 647 | MLANA_013 | TCAATTTACATTTCAGGATAA |
| 648 | MLANA_014 | CATTTCAGGATAAAAGTCTTC |
| 649 | MLANA_015 | AGGATAAAAGTCTTCATGTTG |
| 650 | MLANA_016 | CTGTCCCGATGATCAAACCCT |
| 651 | MLANA_017 | TCTTGAAGAGACACTTTGCTG |
| 652 | MLANA_018 | ATCATCGGGACAGCAAAGTGT |
| 653 | MLANA_019 | TTGTTCTCACAGGTTCCCAAT |
| 654 | MLANA_020 | TCATAAGCAGGTGGAGCATTG |
| 655 | CD3D_001 | TCTCTGGCCTGGTACTGGCTA |
| 656 | CD3D_002 | CCCTTTAGTGAGCCCCTTCAA |
| 657 | CD3D_003 | GTGAGCCCCTTCAAGATACCT |
| 658 | CD3D_004 | TGAATTGCAATACCAGCATCA |
| 659 | CD3D_005 | CCAGGTCCAGTCTTGTAATGT |
| 660 | CD3D_006 | TCCTTGTATATATCTGTCCCA |
| 661 | CD3D_007 | GGAGTCTTCTGCTTTGCTGGA |
| 662 | CD3D_008 | CTGGACATGAGACTGGAAGGC |
| 663 | CD3D_009 | TCTTCTCCTCTCTTAGCCCCT |
| 664 | CD3D_010 | CTCCAAGGTGGCTGTACTGAG |
| 665 | CD3G_001 | CCGGAGGACAGAGACTGACAT |
| 666 | CD3G_002 | TCATTTCAGGAAACCACTTGG |
| 667 | CD3G_003 | AGGAAACCACTTGGTTAAGGT |
| 668 | CD3G_004 | GCTTCTGCATCACAAGTCAGA |
| 669 | CD3G_005 | AACCATGTGATATTTTTGGCT |
| 670 | CD3G_006 | TCTTCAGTTAGGAAGCCGATC |
| 671 | CD3G_007 | AAGATGGGAAGATGATCGGCT |
| 672 | CD3G_008 | CACTGATACATCCCTCGAGGG |
| 673 | CD3G_009 | ACTTGTTCTGTGATCCTTTAC |
| 674 | CD3G_010 | TCTCTCCTTTTCCCTACAGTG |
| 675 | CD3G_011 | GTTCAATGCAGTTCTGACACA |
| 676 | CD3G_012 | CCTACAGTGTGTCAGAACTGC |
| 677 | CD3G_013 | AGCAAAGAGAAAGCCAGATAT |
| 678 | CD3G_014 | TCTTTGCTGAAATCGTCAGCA |
| 679 | CD3G_015 | CTGAAATCGTCAGCATTTTCG |
| 680 | CD3G_016 | GTCCTTGCTGTTGGGGTCTAC |
| 681 | CD3G_017 | CCTCTCGACTGGCGAACTCCA |
| 682 | CD3G_018 | ttttttgTGCAGCTTCAGACA |
| 683 | CD3G_019 | TGCAGCTTCAGACAAGCAGAC |
| 684 | CD3G_020 | TTCTTCATCCCCTTACCTGGT |
| 685 | CD3G_021 | CAGCCCCTCAAGGATCGAGAA |
| 686 | CD3G_022 | CTTGAAGGTGGCTGTACTGGT |
| 687 | CD3G_023 | CAGGTACTTTGGCCCAGTCAA |
| 688 | CD247_001 | TGAGGGAAAGGACAAGATGAA |
| 689 | CD247_002 | ACCGCGGCCATCCTGCAGGCA |
| 690 | CD247_003 | TCTCTTGGCACAGAGGCACAG |
| 691 | CD247_004 | GGATCCAGCAGGCCAAAGCTC |
| 692 | CD247_005 | GCCTGCTGGATCCCAAACTCT |
| 693 | CD247_006 | CTTTCTGTGTTGCAGTTCAGC |
| 694 | CD247_007 | TGTGTTGCAGTTCAGCAGGAG |
| 695 | CD247_008 | TTATCTGTTATAGGAGCTCAA |
| 696 | CD247_009 | CCCCCATCTCAGGGTCCCGGC |
| 697 | CD247_010 | GACAAGAGACGTGGCCGGGAC |
| 698 | CD247_011 | CTAGCAGAGAAGGAAGAACCC |
| 699 | CD247_012 | ATCCCAATCTCACTGTAGGCC |
| 700 | CD247_013 | ACTCCCAAACAACCAGCGCCG |
| 701 | CD247_014 | TGATTTGCTTTCACGCCAGGG |
| 702 | CD247_015 | CTTTCACGCCAGGGTCTCAGT |
| 703 | CD247_016 | ACGCCAGGGTCTCAGTACAGC |
| 704 | SOX10_001 | CTGGCGCCGTTGACGCGCACG |
| 705 | SOX10_002 | TTGTGCTGCATACGGAGCCGC |
| 706 | SOX10_003 | ATGTGGCTGAGTTGGACCAGT |
| 707 | SOX10_004 | GCATCCACACCAGGTGGTGAG |
| 708 | SOX10_005 | ACTACTCTGACCATCAGCCCT |
| 709 | SOX10_006 | GGGCCGGGACAGTGTCGTATA |
| 710 | RPL23_001 | ttttttCCGGCGTTCAAGATG |
| 711 | RPL23_002 | CGGCGTTCAAGATGTCGAAGC |
| 712 | RPL23_003 | GCACCAGAGGACCCACCACGT |
| 713 | RPL23_004 | TATCCACAGGACGTGGTGGGT |
| 714 | RPL23_005 | CTTGGGTCTTCCGGTAGGAGC |
| 715 | RPL23_006 | tttacattcttttGTAGGAGC |
| 716 | RPL23_007 | cattcttttGTAGGAGCCAAA |
| 717 | RPL23_008 | TAGGAGCCAAAAACCTGTATA |
| 718 | RPL23_009 | TTGACTGTGGCCATCACCATG |
| 719 | RPL23_010 | CCTTTCTTGACTGTGGCCATC |
| 720 | RPL23_011 | TGAGCTCTGGTTTGCCTTTCT |
| 721 | RPL23_012 | CTCACCCTTTTTTCTGAGCTC |
| 722 | RPL23_013 | GTTGTCGAATGACCACTGCTG |
| 723 | RPL23_014 | TTCTCTCAGTACATCCAGCAG |
| 724 | RPL23_015 | TACGGTATGACTTTCGTTGTC |
| 725 | RPL23_016 | TTGTTCACTATGACTCCTGCA |
| 726 | RPL23_017 | TTTATTTTGAAGATAATGCAG |
| 727 | RPL23_018 | TTTTGAAGATAATGCAGGAGT |
| 728 | RPL23_019 | AAGATAATGCAGGAGTCATAG |
| 729 | RPL23_020 | ATCTCGCCTTTATTGTTCACT |
| 730 | RPL23_021 | CTACCTTTCATCTCGCCTTTA |
| 731 | RPL23_022 | ttttatttttttaATGCAGGT |
| 732 | RPL23_023 | tttttttaATGCAGGTTCTGC |
| 733 | RPL23_024 | CTACTGGTCCTGTAATGGCAG |
| 734 | RPL23_025 | ATGCAGGTTCTGCCATTACAG |
| 735 | RPL23_026 | CAAATATACTGGAGAATCATG |
| 736 | RPL23_027 | CCTTCCCTTTATATCCACAGG |
| 737 | PTCD2_001 | GGCCCTCGAATCGAGTTCTCC |
| 738 | PTCD2_002 | GTGTATCCTGGGGTGGGAGGC |
| 739 | PTCD2_003 | TTTCTCTGATTTTTAGCTAAA |
| 740 | PTCD2_004 | TCTGATTTTTAGCTAAAAGAT |
| 741 | PTCD2_005 | ACCACATTATCTGTAAGTAGG |
| 742 | PTCD2_006 | ATTTCACCACATTATCTGTAA |
| 743 | PTCD2_007 | GCTAAAAGATACCTACTTACA |
| 744 | PTCD2_008 | TTGAAATTCTTTTAATTTCAC |
| 745 | PTCD2_009 | TTTTGTTGAAATTCTTTTAAT |
| 746 | PTCD2_010 | AACAAAAGAAAGTGGCTGTTG |
| 747 | PTCD2_011 | GTGCCAGAAAGATTACATGCA |
| 748 | PTCD2_012 | AAGTTTCTAAAATACGTTTCT |
| 749 | PTCD2_013 | TTTTTCAAGTTTCTAAAATAC |
| 750 | PTCD2_014 | TTCCAGAAACGTATTTTAGAA |
| 751 | PTCD2_015 | GAAACTTGAAAAAGAAACTGA |
| 752 | PTCD2_016 | GCCAGTTCCACATGGTCCCGA |
| 753 | PTCD2_017 | TGTGAGTCTCGGGACCATGTG |
| 754 | PTCD2_018 | ATTACCAGGTACCATGCAGAG |
| 755 | PTCD2_019 | TACTCCCCCAAAGTGAAATTT |
| 756 | PTCD2_020 | ACTTTGGGGGAGTATAAATTT |
| 757 | PTCD2_021 | GGGGAGTATAAATTTGGACCG |
| 758 | PTCD2_022 | GACCGCTTTTTGTGAGGTTGT |
| 759 | PTCD2_023 | TGAGGTTGTGTTACGAGTTGG |
| 760 | PTCD2_024 | ATGAGCTCCACTGCAGATTCC |
| 761 | PTCD2_025 | CGAGGTTTCTTCTCAGACTCC |
| 762 | PTCD2_026 | TTCTCAGACTCCACATCATTC |
| 763 | PTCD2_027 | ATAAATAACATATCCATCAAA |
| 764 | PTCD2_028 | CCTTTGATAAATAACATATCC |
| 765 | PTCD2_029 | TATTTGCCTTTGATAAATAAC |
| 766 | PTCD2_030 | ATGGATATGTTATTTATCAAA |
| 767 | PTCD2_031 | TCAAAGGCAAATATAAAAGTA |
| 768 | PTCD2_032 | ATCTCTATCAATACTTGCAAA |
| 769 | PTCD2_033 | GCAGGTGCTTTGCAAGTATTG |
| 770 | PTCD2_034 | CAAGTATTGATAGAGATGAAA |
| 771 | PTCD2_035 | GTGAACTTCACATCTTGGTTT |
| 772 | PTCD2_036 | TAGCAAATTGCAAAAGCAAGA |
| 773 | PTCD2_037 | CAATTTGCTACAAACTGGTAA |
| 774 | PTCD2_038 | AAAGACTCAGGGCTATTCTGT |
| 775 | PTCD2_039 | AGTAGAGCTTCTTCTCTTAAT |
| 776 | PTCD2_040 | AAAATCTGTACTACATTAAGA |
| 777 | PTCD2_041 | TCCTTTGAGTAGAGCTTCTTC |
| 778 | PTCD2_042 | CCTGATTCAGAGCTAATGCCA |
| 779 | PTCD2_043 | GCTGTGGCATTAGCTCTGAAT |
| 780 | PTCD2_044 | TTTCTCTTCCTTCTAGAATGA |
| 781 | PTCD2_045 | TCTTCCTTCTAGAATGAGATG |
| 782 | PTCD2_046 | AGAAAAAATGGACACAGCTTT |
| 783 | PTCD2_047 | TGGATTCATGATTTGAGAAAA |
| 784 | PTCD2_048 | TCAAATCATGAATCCAGAAAG |
| 785 | PTCD2_049 | ACTGGATATGGATTATAATCT |
| 786 | PTCD2_050 | CAACATATTTGACTGGATATG |
| 787 | PTCD2_051 | TCAGGTTTTCCAACATATTTG |
| 788 | PTCD2_052 | GAGTCTTTATCAGGTTTTCCA |
| 789 | PTCD2_053 | CTTCTGCAGCATTTTTTAGAG |
| 790 | PTCD2_054 | ATAAATTTCCTTCTGCAGCAT |
| 791 | PTCD2_055 | ACAAATTTTGATAAATTTCCT |
| 792 | PTCD2_056 | TCAAAATTTGTGAAAAGACAT |
| 793 | PTCD2_057 | TGAAAAGACATGTGTTCTCGG |
| 794 | PTCD2_058 | TGCAGCACTTGCATACTCACC |
| 795 | PTCD2_059 | CAGCTGGCCAAAGTGAGGGAA |
| 796 | PTCD2_060 | GCCACAAGGGCAGGCACATCC |
| 797 | PTCD2_061 | ATGAGATCTATGGGACACTGC |
| 798 | PTCD2_062 | CTGTCCCTGGGGGTGTGGCAG |
| 799 | PTCD2_063 | GATGCTGTGCTCTGCCACACC |
| 800 | PTCD2_064 | ATAGCAACGTGTGAGATTTCC |
| 801 | SRP54_001 | TTCCAAGGTCTGCTAGAACCA |
| 802 | SRP54_002 | AATTTCATTTATTTCTTTATT |
| 803 | SRP54_003 | GCATAGCATTCAATACCTGAA |
| 804 | SRP54_004 | ATTTATTTCTTTATTTTCAGG |
| 805 | SRP54_005 | TTTCTTTATTTTCAGGTATTG |
| 806 | SRP54_006 | TTTATTTTCAGGTATTGAATG |
| 807 | SRP54_007 | TTTTCAGGTATTGAATGCTAT |
| 808 | SRP54_008 | AGGTATTGAATGCTATGCTAA |
| 809 | SRP54_009 | ATATTAACATCTGCTTCCAAC |
| 810 | SRP54_010 | TTGGAAGCAGATGTTAATATT |
| 811 | SRP54_011 | TCTTAGTTGCTTCACTAGTTT |
| 812 | SRP54_012 | TGATGGTGGTTGGTGATTGGG |
| 813 | SRP54_013 | TTAAGACCAGATGCCATCTCT |
| 814 | SRP54_014 | TTTTGTTAAGACCAGATGCCA |
| 815 | SRP54_015 | AATACAGCATGCTGAATCATT |
| 816 | SRP54_016 | tttttaatttatttttggtat |
| 817 | SRP54_017 | atttatttttggtatttaGCT |
| 818 | SRP54_018 | tttttggtatttaGCTTGTAG |
| 819 | SRP54_019 | gtatttaGCTTGTAGACCCTG |
| 820 | SRP54_020 | GTGGGTGTCCATGCCTTAACT |
| 821 | SRP54_021 | GCTTGTAGACCCTGGAGTTAA |
| 822 | SRP54_022 | CTTTAGTGGGTGTCCATGCCT |
| 823 | SRP54_023 | TTTTCCTTTAGTGGGTGTCCA |
| 824 | SRP54_024 | CCACTCCCTTGCAATCCAACA |
| 825 | SRP54_025 | AACATGTTGTTGTTTTACCAC |
| 826 | SRP54_026 | TTGGATTGCAAGGGAGTGGTA |
| 827 | SRP54_027 | CTCTGGTAATAATATGCTAGC |
| 828 | SRP54_028 | AATCTTTTCTCACCCAGCTAG |
| 829 | SRP54_029 | TCACCCAGCTAGCATATTATT |
| 830 | SRP54_030 | ATATGTGCAGACACATTCAGA |
| 831 | SRP54_031 | TTTTCAAGTTTGAGGATTCAT |
| 832 | SRP54_032 | AAGTTTGAGGATTCATGAACT |
| 833 | SRP54_033 | AGGATTCATGAACTCTTTATC |
| 834 | SRP54_034 | GTTGGTCAAAAGCCCCTGGAA |
| 835 | SRP54_035 | TCTTCCAGGGGCTTTTGACCA |
| 836 | SRP54_036 | GTAGCATTCTGTTTTAGTTGG |
| 837 | SRP54_037 | ACCAACTAAAACAGAATGCTA |
| 838 | SRP54_038 | TGTATAGCTATATAACATGGA |
| 839 | SRP54_039 | TTAAATCATTTGTCCATGTTA |
| 840 | SRP54_040 | TCCATGTTATATAGCTATACA |
| 841 | SRP54_041 | TCTACTCCTTCAGAAGCAATG |
| 842 | SRP54_042 | AATTTCTCTACTCCTTCAGAA |
| 843 | SRP54_043 | ATTTTTAAATTTCTCTACTCC |
| 844 | SRP54_044 | AAAATTTTCATTTTTAAATTT |
| 845 | SRP54_045 | AAAATGAAAATTTTGAAATTA |
| 846 | SRP54_046 | TGGCGGCCACTTGTATCAACA |
| 847 | SRP54_047 | AAATTATTATTGTTGATACAA |
| 848 | SRP54_048 | TTCAAACAAAGAGTCTTCTTG |
| 849 | SRP54_049 | TTTGAAGAAATGCTTCAAGTT |
| 850 | SRP54_050 | AAGAAATGCTTCAAGTTGCTA |
| 851 | SRP54_051 | TATTTAAACTTTCTAGCAACC |
| 852 | SRP54_052 | AACTTTCTAGCAACCTGATAA |
| 853 | SRP54_053 | TAGCAACCTGATAACATTGTT |
| 854 | SRP54_054 | TGTGATGGATGCCTCCATTGG |
| 855 | SRP54_055 | AAAGCCTTAGCCTGGGCTTCA |
| 856 | SRP54_056 | TCTTTAAAAGCCTTAGCCTGG |
| 857 | SRP54_057 | TCACTATTACTGAGGCTACAT |
| 858 | SRP54_058 | AAGATAAAGTAGATGTAGCCT |
| 859 | SRP54_059 | CATGGCCATCAAGTTTTGTCA |
| 860 | SRP54_060 | TGGCAGCGACTCTGGaaaaaa |
| 861 | SRP54_061 | tattttctttttttttCCAGA |
| 862 | SRP54_062 | tttttttttCCAGAGTCGCTG |
| 863 | SRP54_063 | CAGAGTCGCTGCCACAAAAAG |
| 864 | SRP54_064 | ATTGGTACAGGGGAACATATA |
| 865 | SRP54_065 | AAAGGTTCAAAGTCATCTATA |
| 866 | SRP54_066 | CTAATAAAAGGCTGTGTTTTG |
| 867 | SRP54_067 | AACCTTTCAAAACACAGCCTT |
| 868 | SRP54_068 | AAAACACAGCCTTTTATTAGC |
| 869 | SRP54_069 | TTTTTTGTATCTTATAGGTAT |
| 870 | SRP54_070 | TATCTTATAGGTATGGGCGAC |
| 871 | SRP54_071 | TCTATCAGTCCTTCAATGTCG |
| 872 | SRP54_072 | AACTTCTCTATAAGTGCTTCA |
| 873 | SRP54_073 | CATTGTATTTCAGGTCAGTTT |
| 874 | SRP54_074 | AAATTGCTCATACATGTCTCG |
| 875 | SRP54_075 | AGGTCAGTTTACGTTGCGAGA |
| 876 | SRP54_076 | ATGATATTTTGAAATTGCTCA |
| 877 | SRP54_077 | CGTTGCGAGACATGTATGAGC |
| 878 | SRP54_078 | AAAATATCATGAAAATGGGCC |
| 879 | SRP54_079 | TGTTTAAATCTGTTGTAGGGG |
| 880 | SRP54_080 | AATCTGTTGTAGGGGATGATC |
| 881 | SRP54_081 | CTCATAAAATCTGTCCCAAAA |
| 882 | SRP54_082 | CTTTGCTCATAAAATCTGTCC |
| 883 | SRP54_083 | GGACAGATTTTATGAGCAAAG |
| 884 | SRP54_084 | GCCTTGCCATTGACTCCTGTT |
| 885 | SRP54_085 | TGAGCAAAGGAAATGAACAGG |
| 886 | SRP54_086 | TTTAGCCTTGCCATTGACTCC |
| 887 | SRP54_087 | GCACCATCCGTACTGTCTAGT |
| 888 | SRP54_088 | CTAAAAACTTTGGCACCATCC |
| 889 | SRP54_089 | GATTCTTCCTGGTTGTTTACT |
| 890 | SRP54_090 | GTAAACAACCAGGAAGAATCC |
| 891 | SRP54_091 | CCATCTGTGCAAACTTGGTAT |
| 892 | SRP54_092 | ACACAATATACCAAGTTTGCA |
| 893 | SRP54_093 | ATACCTCCCATCTTTTTTACC |
| 894 | SRP54_094 | CACAGATGGTAAAAAAGATGG |
| 895 | SRP54_095 | AAAAGTCCTTTGATACCTCCC |
| 896 | SRP54_096 | CCCTCAGGTGGCGACATGTCT |
| 897 | SRP54_097 | CCATCTGTGACTGGCTCACAT |
| 898 | SRP54_098 | TTGGTTCAATTTTGCCATCTG |
| 899 | SRP54_099 | GCCATTTGTTGGTTCAATTTT |
| 900 | SRP54_100 | ACTCTACTTCCCTACTTTTGC |
| 901 | SRP54_101 | CTCTAGGTGGTATGGCAGGAC |
| 902 | SRP54_102 | ATGTTGCCAGCAGCACCCTGT |
| 903 | SRP54_103 | AACAGGGTGCTGCTGGCAACA |
| 904 | SRP54_104 | CATATTATTGAATCCCATCAT |
| 905 | SRP54_105 | TTTACATATTATTGAATCCCA |
| 906 | SRP54_106 | TATTAAGGCATTTTCTTTACA |
| 907 | SRP54_107 | CTGAGACCTCAGCGTTTCCCT |
| 908 | SRP54_108 | CCCCCAATTCGCAAAAAGAAG |
| 909 | SRP54_109 | CCTTCTTTTTGCGAATTGGGG |
| 910 | SRP54_110 | CGAATTGGGGGGAAAGTGTAT |
| 911 | SRP54_111 | TTGCTTATCATGCACTCTTTC |
| 912 | SRP54_112 | Cttttcttctcgcccgctttt |
| 913 | SRP54_113 | ttctcgcccgcttttcccctc |
| 914 | SRP54_114 | ccctccttttctttttccttc |
| 915 | SRP54_115 | TCCCTTATATTaaagggagga |
| 916 | SRP54_116 | tttttccttccttctttcctc |
| 917 | SRP54_117 | cttccttctttcctccctttA |
| 918 | SRP54_118 | ctccctttAATATAAGGGAGA |
| 919 | SRP54_119 | CACAAAAACCATGTATTTCTC |
| 920 | SRP54_120 | ATATAAGGGAGAAATACATGG |
| 921 | SRP54_121 | TGGAAATCATTATATGTTTGC |
| 922 | SRP54_122 | CTTTAGATTTTCTTCTGTTTT |
| 923 | SRP54_123 | ACTTAAGTGTTATGATGGTGA |
| 924 | SRP54_124 | GATTTTCTTCTGTTTTCACCA |
| 925 | SRP54_125 | TTCTGTTTTCACCATCATAAC |
| 926 | SRP54_126 | CATCATGATTTAACTTAAGTG |
| 927 | SRP54_127 | ACCATCATAACACTTAAGTTA |
| 928 | SRP54_128 | AGTACTAAAATTTTACATCAT |
| 929 | SRP54_129 | GTACTTAAAGGTTTTTAATTA |
| 930 | SRP54_130 | CAAATGCAATGCTTGGCCTTC |
| 931 | SRP54_131 | ATTATCTCGAAGGCCAAGCAT |
| 932 | SRP54_132 | ACTACTGACCAGGACTGTTTA |
| 933 | SRP54_133 | ATTGAAACATTATTTAACTAC |
| 934 | SRP54_134 | TAAACAGTCCTGGTCAGTAGT |
| 935 | SRP54_135 | CAGCACTTTAATTGAAACATT |
| 936 | SRP54_136 | TTTTACAGCACTTTAATTGAA |
| 937 | SRP54_137 | AAGTTTATTTTACAGCACTTT |
| 938 | SRP54_138 | AATTAAAGTGCTGTAAAATAA |
| 939 | SRP54_139 | AGGATAACTAACCAAGATCTG |
| 940 | ERAP2_001 | TGTGTGAATTAACCATTGCAG |
| 941 | ERAP2_002 | ATGTTCCATTCTTCTGCAATG |
| 942 | ERAP2_003 | ACATTCACAGAGGATTTTACT |
| 943 | ERAP2_004 | GGGCAAGATGGCTGTTAAGCA |
| 944 | ERAP2_005 | CTGCTTAACAGCCATCTTGCC |
| 945 | ERAP2_006 | TTCTCAGTTCTCAGTGCCATC |
| 946 | ERAP2_007 | CCAGTAGCCACTAATGGGGAA |
| 947 | ERAP2_008 | CTTGGCAGGAGCTAAGGCTCC |
| 948 | ERAP2_009 | TCCACCCCAATCTCACCTCTC |
| 949 | ERAP2_010 | TTGCATCTGAGAAGATCGAAG |
| 950 | ERAP2_011 | CTGTGCAAGATGATAAACTGG |
| 951 | ERAP2_012 | AAGATCTTTGCTGTGCAAGAT |
| 952 | ERAP2_013 | TCATCTTGCACAGCAAAGATC |
| 953 | ERAP2_014 | ATGTATCTTGAATCTTCCTCT |
| 954 | ERAP2_015 | CTGGTTTCATGTATCTTGAAT |
| 955 | ERAP2_016 | AGTTCTTTTCCTGGTTTCATG |
| 956 | ERAP2_017 | TTCATGAGCAGGGTAACTCAA |
| 957 | ERAP2_018 | AGTTACCCTGCTCATGAACAA |
| 958 | ERAP2_019 | TCTGGAACCAGCAGTGCAATT |
| 959 | ERAP2_020 | AGGTGAGGCGTAAGTTTCTCT |
| 960 | ERAP2_021 | TAAAACCCTTCAAAGCCATCA |
| 961 | ERAP2_022 | AAGGGTTTTATAAAAGCACAT |
| 962 | ERAP2_023 | ACCACCAAGAGTTCTGTATGT |
| 963 | ERAP2_024 | TAAAAGCACATACAGAACTCT |
| 964 | ERAP2_025 | GCTGGGGGGGGTCTTTTCAC |
| 965 | ERAP2_026 | ACAGAATTCTTGCAGTAACAG |
| 966 | ERAP2_027 | AGCCAACCCAGGCACGCATGG |
| 967 | ERAP2_028 | AACAACGGTTCATCAAAGCAA |
| 968 | ERAP2_029 | CCTTGCTTTGATGAACCGTTG |
| 969 | ERAP2_030 | ATGAACCGTTGTTCAAAGCCA |
| 970 | ERAP2_031 | AATCAAGATACGAAGAGAGAG |
| 971 | ERAP2_032 | GCATGTTGGATAGTGCAATAT |
| 972 | ERAP2_033 | CTTTCTGTAGGTTAAGACAAT |
| 973 | ERAP2_034 | TGTAGGTTAAGACAATTGAAC |
| 974 | ERAP2_035 | AAAGTGATCTTCCAAAAGACC |
| 975 | ERAP2_036 | CAGTAGTTTCAAAGTGATCTT |
| 976 | ERAP2_037 | GAAGATCACTTTGAAACTACT |
| 977 | ERAP2_038 | AAACTACTGTAAAAATGAGTA |
| 978 | ERAP2_039 | TGATTTCCACTCTCTGAGTGG |
| 979 | ERAP2_040 | CACTCTCTGAGTGGCTTCACT |
| 980 | ERAP2_041 | TCTGGGGATGCATAGATGGAC |
| 981 | ERAP2_042 | ATTCCGTTTGTCTGGGGATGC |
| 982 | ERAP2_043 | CCAGGTGTCCATCTATGCATC |
| 983 | ERAP2_044 | ATAAAAATCAAGTAGCTTCAG |
| 984 | ERAP2_045 | CAGGCATCACTGAAGCTACTT |
| 985 | ERAP2_046 | GAGAGTGGATAGTAGATATCA |
| 986 | ERAP2_047 | TGAAAAGTACTTTGATATCTA |
| 987 | ERAP2_048 | ATATCTACTATCCACTCTCCA |
| 988 | ERAP2_049 | TTAGATTTAATTGCTATTCCT |
| 989 | ERAP2_050 | CATGGCTCCAGGTGCAAAGTC |
| 990 | ERAP2_051 | ATTGCTATTCCTGACTTTGCA |
| 991 | ERAP2_052 | CACCTGGAGCCATGGAAAATT |
| 992 | ERAP2_053 | TCGGAAGCAGAAGAGGTCTTG |
| 993 | ERAP2_054 | ACCCCAAGACCTCTTCTGCTT |
| 994 | ERAP2_055 | CCTCCTAGTGGTTTGGCAACC |
| 995 | ERAP2_056 | GCAACCTGGTCACAATGGAAT |
| 996 | ERAP2_057 | CAAAACCCTCCTTAAGCCAAA |
| 997 | ERAP2_058 | GCTTAAGGAGGGTTTTGCAAA |
| 998 | ERAP2_059 | CAAAATACATGGAACTTATCG |
| 999 | ERAP2_060 | TCCCTGTTTAGGATGACTATT |
| 1000 | ERAP2_061 | GGATGACTATTTTTTGAATGT |
| 1001 | ERAP2_062 | TAATTACTTCAAAACACACAT |
| 1002 | ERAP2_063 | AATGTGTGTTTTGAAGTAATT |
| 1003 | ERAP2_064 | AAGTAATTACAAAAGATTCAT |
| 1004 | ERAP2_065 | GAGATAGGGCGGGATGAATTC |
| 1005 | ERAP2_066 | CGCTGGTTTGGAGATAGGGCG |
| 1006 | ERAP2_067 | AGTCGGGGTTTCCGCTGGTTT |
| 1007 | ERAP2_068 | CTGTATTTGAGTCGGGGTTTC |
| 1008 | ERAP2_069 | aaaaaaaacaaaagagttgaa |
| 1009 | ERAP2_070 | aactcttttgtttttttttAA |
| 1010 | ERAP2_071 | tttttttttAAAGGGAGCTTG |
| 1011 | ERAP2_072 | AAGGGAGCTTGTATTTTGAAT |
| 1012 | ERAP2_073 | TCCTCACCCAGAAAATCCTTG |
| 1013 | ERAP2_074 | AATATGCTCAAGGATTTTCTG |
| 1014 | ERAP2_075 | TGGAATTTCTCCTCACCCAGA |
| 1015 | ERAP2_076 | TGGGTGAGGAGAAATTCCAGA |
| 1016 | ERAP2_077 | AGTACTGAATTATTCCTTTCT |
| 1017 | ERAP2_078 | TATAGCTGAACTTCTTTAAGT |
| 1018 | ERAP2_079 | ACAGACTGCTCCACAAGTCAT |
| 1019 | ERAP2_080 | TAAACAACTCTACAAAACAAG |
| 1020 | ERAP2_081 | TTCTTGTTTTGTAGAGTTGTT |
| 1021 | ERAP2_082 | TAGAGTTGTTTAGAAAGTGAT |
| 1022 | ERAP2_083 | GAAAGTGATTTTACATCTGGT |
| 1023 | ERAP2_084 | CATCTGGTGGAGTTTGTCATT |
| 1024 | ERAP2_085 | TCATTCGGATCCCAAGATGAC |
| 1025 | ERAP2_086 | CCCCAGAAAGGCGAGCTGAAA |
| 1026 | ERAP2_087 | ACCTCTGCATTTTCCCCCAGA |
| 1027 | ERAP2_088 | AGCTCGCCTTTCTGGGGGAAA |
| 1028 | ERAP2_089 | TGGGGGAAAATGCAGAGGTCA |
| 1029 | ERAP2_090 | TGGAGAGTCCATGTAGTCATC |
| 1030 | ERAP2_091 | ACCACCAGCAGGGGGATTCCT |
| 1031 | ERAP2_092 | CAGGAAGACCCTGAATGGAGG |
| 1032 | ERAP2_093 | CTCTCTGTCATAGGTACCTGT |
| 1033 | ERAP2_094 | GAATGTGTCTGTGGATCACAT |
| 1034 | ERAP2_095 | ATTTTAGAATGTGTCTGTGGA |
| 1035 | ERAP2_096 | AGGTAGATCCAGAGTATCTAA |
| 1036 | ERAP2_097 | ACCCAACTGGTCTTTTCAGGT |
| 1037 | ERAP2_098 | AGTCCACATTAAATTTCACCC |
| 1038 | ERAP2_099 | ATGTGGACTCAAATGGTTACT |
| 1039 | ERAP2_100 | TCTAGGGTCAGTCTCCCTGCA |
| 1040 | ERAP2_101 | TTTTATACTTCAGTGCAGGGA |
| 1041 | ERAP2_102 | TACTTCAGTGCAGGGAGACTG |
| 1042 | ERAP2_103 | ATGTTGGAGGTAGTAAGTCAT |
| 1043 | ERAP2_104 | AGAGATATCTGAAATATTCCT |
| 1044 | ERAP2_105 | CCACATGATGGACAGAAGGAA |
| 1045 | ERAP2_106 | AGATATCTCTGAAAACCTCAA |
| 1046 | ERAP2_107 | TGCAGCGTTACCTTCTTCAGT |
| 1047 | ERAP2_108 | CCTGTCAATCACTGGCTTAAA |
| 1048 | ERAP2_109 | AGCCAGTGATTGACAGGCAAA |
| 1049 | ERAP2_110 | TGGATGCAAGGAGCATGGTTC |
| 1050 | ERAP2_111 | CACTGGATTCCATCCACTGGG |
| 1051 | ERAP2_112 | ATTTTCCACTGGATTCCATCC |
| 1052 | ERAP2_113 | TTCATTTTTATGCTTGATATT |
| 1053 | ERAP2_114 | AAACATCTGTTGGTATACTGT |
| 1054 | ERAP2_115 | TGCTTGATATTACAGTATACC |
| 1055 | ERAP2_116 | AAGATTGTGTATTCTGTGGGT |
| 1056 | ERAP2_117 | TTCAGCACTTGACATTGACAG |
| 1057 | ERAP2_118 | GAGCAATATGAACTGTCAATG |
| 1058 | ERAP2_119 | TTTTGTTCAGCACTTGACATT |
| 1059 | ERAP2_120 | CTGATGCTTGCTCGTTGACAA |
| 1060 | ERAP2_121 | TCAACGAGCAAGCATCAGGAA |
| 1061 | ERAP2_122 | GAAACAAATTATTTTTCTTTC |
| 1062 | ERAP2_123 | CTTCCATTCCTAGTTCAATTA |
| 1063 | ERAP2_124 | TTTCTTCAGGTTAATTGAACT |
| 1064 | ERAP2_125 | TTCAGGTTAATTGAACTAGGA |
| 1065 | ERAP2_126 | GACGTCTGGCAATCGCATGAA |
| 1066 | ERAP2_127 | TCTTACAAAATCCCATGCTAG |
| 1067 | ERAP2_128 | AGAAGATGGGTCCAATTTTCT |
| 1068 | ERAP2_129 | TAAGAGAAAATTGGACCCATC |
| 1069 | ERAP2_130 | TATGGTGTTTCTTTTTATTTT |
| 1070 | ERAP2_131 | TTTTTATTTTCAGATTTGACT |
| 1071 | ERAP2_132 | TTTTCAGATTTGACTTGGGCT |
| 1072 | ERAP2_133 | AGATTTGACTTGGGCTCATAT |
| 1073 | ERAP2_134 | ACTTGGGCTCATATGACATAA |
| 1074 | ERAP2_135 | TTCCAAGGATAAGTTGCAAGA |
| 1075 | ERAP2_136 | ACCTGTAAAATATTGAAGAAA |
| 1076 | ERAP2_137 | TTCAATATTTTACAGGTGAAA |
| 1077 | ERAP2_138 | CAGGTGAAACTATTTTTTGAA |
| 1078 | ERAP2_139 | AATCTCTTGAGGCTCAAGGAT |
| 1079 | ERAP2_140 | AAAAATATCCAGATGTGATCC |
| 1080 | ERAP2_141 | CAGAACAGTTTGAAAAATATC |
| 1081 | ERAP2_142 | GTTATCGTTTCCAGAACAGTT |
| 1082 | ERAP2_143 | TATTTTTGGTTATCGTTTCCA |
| 1083 | ERAP2_144 | AAACTGTTCTGGAAACGATAA |
| 1084 | ERAP2_145 | AGTATTAACCATTAGCCAAGT |
| 1085 | ERAP2_146 | TATTGACCATTTAAGTATTAA |
| 1086 | ERAP2_147 | ggaggctgagaagggcggatc |
| 1087 | ERAP2_148 | gcggagacggggtctcaccgt |
| 1088 | ERAP2_149 | tatttttagcggagacggggt |
| 1089 | ERAP2_150 | ctgctgcagcctgccgagtag |
| 1090 | ERAP2_151 | tgccattttcctgctgcagcc |
| 1091 | ERAP2_152 | agacagagtctcgctcagtca |
| 1092 | ERAP2_153 | ACTTCATGCAGGCAGTCATGT |
| 1093 | ERAP2_154 | TTGAGACTTCTTGTTGGTTAG |
| 1094 | ERAP2_155 | TCTAACCAACAAGAAGTCTCA |
| 1095 | ERAP2_156 | GCTGGATACGATAGCTGAGAG |
| 1096 | ERAP2_157 | AATAGGATACTGAACTGGCCT |
| 1097 | ERAP2_158 | CTTTTAAATAGGATACTGAAC |
| 1098 | ERAP2_159 | AAAGTAAACTTCCTGAATAAT |
| 1099 | ERAP2_160 | GCCTCAAGTGACTTTCTCCAT |
| 1100 | ERAP2_161 | TCCATTGCTTCACGCTATGCC |
| 1101 | ERAP2_162 | CTTCTTTAATTTTTTTAACCT |
| 1102 | ERAP2_163 | ATTTTTTTAACCTTGCTTAGT |
| 1103 | ERAP2_164 | ACCTTGCTTAGTATTCTATAG |
| 1104 | ERAP2_165 | CTTGGACGTAAAACTGGTTGG |
| 1105 | ERAP2_166 | CCCAACCAGTTTTACGTCCAA |
| 1106 | ERAP2_167 | TGCATTGGCTAATTTTCCTTG |
| 1107 | ERAP2_168 | tataTTTTATGCATTGGCTAA |
| 1108 | ERAP2_169 | CGTCCAAGGAAAATTAGCCAA |
| 1109 | ERAP2_170 | atagtttgtataTTTTATGCA |
| 1110 | ERAP2_171 | ctgatccttgcctttcatagt |
| 1111 | ERAP2_172 | gtggcaaagtctctggtttcc |
| 1112 | ERAP2_173 | taataatctgagatttggtgg |
| 1113 | ERAP2_174 | ccaccaaatctcagattatta |
| 1114 | ERAP2_175 | tcaagaaggccaggaaggcct |
| 1115 | ERAP2_176 | ggttaagccttacattcatga |
| 1116 | ERAP2_177 | gaatgctctcaaaaatctacc |
| 1117 | ERAP2_178 | accaGAGACCATtcatttgga |
| 1118 | ERAP2_179 | agagcattccaaatgaATGGT |
| 1119 | ERAP2_180 | accattcattcatttgaccaG |
| 1120 | ERAP2_181 | ttcatttgaccattcattcat |
| 1121 | ERAP2_182 | TATCTCTGTGAGGGCAGattt |
| 1122 | ERAP2_183 | CTTTTGTATCTCTGTGAGGGC |
| 1123 | ERAP2_184 | gtttaagccttacattcatga |
| 1124 | ERAP2_185 | agccttacattcatgaagtac |
| 1125 | ERAP2_186 | gaatgatctcaaaaatctacc |
| 1126 | ERAP2_187 | agatcattccaaatgaagtcg |
| 1127 | ERAP2_188 | Ttgtgtctctgtgagggcaga |
| 1128 | ERAP2_189 | TTAAAAATGCAATAGTGTATG |
| 1129 | ERAP2_190 | aaaagatTTATTAAAAATGCA |
| 1130 | ERAP2_191 | ATAAatcttttgaaatttgca |
| 1131 | ERAP2_192 | accgaaaatacacaatacaat |
| 1132 | ERAP2_193 | aaatttgcagaattagattgt |
| 1133 | ERAP2_194 | cagaattagattgtattgtgt |
| 1134 | ERAP2_195 | cattcaattatcatttaaccg |
| 1135 | ERAP2_196 | ggttaaatgataattgaatgt |
| 1136 | ERAP2_197 | gatgcagcaccatattttata |
| 1137 | ERAP2_198 | Cttaaaatatgaagaaatgct |
| 1138 | ERAP2_199 | taacccagctttagcatttct |
| 1139 | ERAP2_200 | gcatttcttcatattttaaGG |
| 1140 | ERAP2_201 | ttcatattttaaGGAAACCCC |
| 1141 | ERAP2_202 | aGGAAACCCCCCACCTCCTTC |
| 1142 | ERAP2_203 | AGAGAGCAAGAAGCGCCCTTA |
| 1143 | ERAP2_204 | GCAGGGCATTTCAGAGAGCAA |
| 1144 | ERAP2_205 | AGGGCGCTTCTTGCTCTCTGA |
| 1145 | ERAP2_206 | ttccaaactaccttattcaaa |
| 1146 | ERAP2_207 | tttattccaaactaccttatt |
| 1147 | ERAP2_208 | tttctttattccaaactacct |
| 1148 | ERAP2_209 | aataaggtagtttggaataaa |
| 1149 | ERAP2_210 | ctatctgtatgtagagtgatc |
| 1150 | ERAP2_211 | gaataaagaaagaaaagatca |
| 1151 | ERAP2_212 | tactgtctcatatataggatc |
| 1152 | ERAP2_213 | tgatcctatatatgagacagt |
| 1153 | ERAP2_214 | taaaacttatctgtattttta |
| 1154 | ERAP2_215 | agtctttctaaaacttatctg |
| 1155 | ERAP2_216 | catattgttttgagtctttct |
| 1156 | ERAP2_217 | gaaagactcaaaacaatatgt |
| 1157 | ERAP2_218 | cattattaaggaagacttggg |
| 1158 | ERAP2_219 | CCCTCTTGACCCaacatccca |
| 1159 | ERAP2_220 | GAGCAATATCATGAAGGTCAA |
| 1160 | ERAP2_221 | TTTGATGCCACAGTCAGAGAT |
| 1161 | ERAP2_222 | ATGCCACAGTCAGAGATAGAA |
| 1162 | ERAP2_223 | GGTGGCCATGGATGTGCCCCA |
| 1163 | ERAP2_224 | ACCAAAAAATGTGTACTGTAT |
| 1164 | ERAP2_225 | GTTAAATTTGTTTTCAGATCA |
| 1165 | ERAP2_226 | TTTTCAGATCATTTCATGGAA |
| 1166 | ERAP2_227 | AGATCATTTCATGGAATCTTT |
| 1167 | ERAP2_228 | ATGGAATCTTTGAAGTATCTT |
| 1168 | ERAP2_229 | AAGTATCTTTGACTCTAACTT |
| 1169 | ERAP2_230 | ACTCTAACTTTGACTTGGTGG |
| 1170 | ERAP2_231 | ACTTGGTGGTGGACCTTCCTT |
| 1171 | ERAP2_232 | TAACACCTAAGAGATATCCTT |
| 1172 | ERAP2_233 | CTTATGCTAAAATACATGTAA |
| 1173 | ERAP2_234 | AATTTCCTTATGCTAAAATAC |
| 1174 | ERAP2_235 | CTTTTTCAATTTCCTTATGCT |
| 1175 | ERAP2_236 | GAATTACATGTATTTTAGCAT |
| 1176 | ERAP2_237 | GCATAAGGAAATTGAAAAAGT |
| 1177 | ERAP2_238 | CATATGGTCTTGTTGAAAAAA |
| 1178 | ERAP2_239 | ATTTACATATGGTCTTGTTGA |
| 1179 | ERAP2_240 | ACTATTTAATTTACATATGGT |
| 1180 | ERAP2_241 | AACAAGACCATATGTAAATTA |
| 1181 | ERAP2_242 | TGGAAACATTGTTGATGGTAC |
| 1182 | ERAP2_243 | AGTAGAACTGTACCATCAACA |
| 1183 | ERAP2_244 | CATAAATATGCAGAGTTCTTT |
| 1184 | ERAP2_245 | ACAATATTGTAAATAACAATA |
| 1185 | ERAP2_246 | TTTTGTATTGTTATTTACAAT |
| 1186 | ERAP2_247 | TATTGTTATTTACAATATTGT |
| 1187 | ERAP2_248 | CAATATTGTTAAATTGAATGC |
| 1188 | ERAP2_249 | GAATCCTAGAAATTGCAAATG |
| 1189 | ERAP2_250 | TGTACTCAATTCTTTAGAATC |
| 1190 | ERAP2_251 | CAATTTCTAGGATTCTAAAGA |
| 1191 | ERAP2_252 | TAGGATTCTAAAGAATTGAGT |
| 1192 | ERAP2_253 | TTATTTGATGATAATATGAGA |
| 1193 | ERAP2_254 | ATGATAATATGAGAATTACTG |
| 1194 | ERAP2_255 | TCAAAACAGTATTGGCACAGT |
| 1195 | ERAP2_256 | TTTATCAAAACAGTATTGGCA |
| 1196 | ERAP2_257 | AAAATCTATTTATTTATCAAA |
| 1197 | ERAP2_258 | TTTTTAAAAATCTATTTATTT |
| 1198 | ERAP2_259 | ATAAATAAATAGATTTTTAAA |
| 1199 | ERAP2_260 | AAAATAAATGTATTGTACTTA |
| 1200 | ERAP2_261 | TCCTTACCATGTTACTTGTCA |
| 1201 | STAT1_001 | CCTATAGGATGTCTCAGTGGT |
| 1202 | STAT1_002 | AGTCAAGCTGCTGAAGTTCGT |
| 1203 | STAT1_003 | CATGGGAAAACTGTCATCATA |
| 1204 | STAT1_004 | TGATGACAGTTTTCCCATGGA |
| 1205 | STAT1_005 | TAACCACTGTGCCAGGTACTG |
| 1206 | STAT1_006 | CCATGGAAATCAGACAGTACC |
| 1207 | STAT1_007 | ATTTGCCACCATCCGTTTTCA |
| 1208 | STAT1_008 | CCACCATCCGTTTTCATGACC |
| 1209 | STAT1_009 | ATGACCTCCTGTCACAGCTGG |
| 1210 | STAT1_010 | CTTATGTTATGCTGTAGCAAG |
| 1211 | STAT1_011 | TTTGGAGAATAACTTCTTGCT |
| 1212 | STAT1_012 | GAGAATAACTTCTTGCTACAG |
| 1213 | STAT1_013 | TTCTAACCACTCAAATCTAGG |
| 1214 | STAT1_014 | AGGAAGACCCAATCCAGATGT |
| 1215 | STAT1_015 | TTCCTTCAGACAGCTGTAAAT |
| 1216 | STAT1_016 | CTTTCTTCCTTCAGACAGCTG |
| 1217 | STAT1_017 | CAGAATTTTCCTTTCTTCCTT |
| 1218 | STAT1_018 | CAGCTGTCTGAAGGAAGAAAG |
| 1219 | STAT1_019 | TCTAACATCACTGTGCTCTGA |
| 1220 | STAT1_020 | TGTTTGTCTAACATCACTGTG |
| 1221 | STAT1_021 | CTGTCAAGCTCTTTCTGTTTG |
| 1222 | STAT1_022 | TGACTTTACTGTCAAGCTCTT |
| 1223 | STAT1_023 | ATGCTCTATACACTACAAACA |
| 1224 | STAT1_024 | CATCTTTGTTTGTAGTGTATA |
| 1225 | STAT1_025 | TTTGTAGTGTATAGAGCATGA |
| 1226 | STAT1_026 | TAGTGTATAGAGCATGAAATC |
| 1227 | STAT1_027 | AAGTCATATTCATCTTGTAAA |
| 1228 | STAT1_028 | CATTTGAAGTCATATTCATCT |
| 1229 | STAT1_029 | CAAGATGAATATGACTTCAAA |
| 1230 | STAT1_030 | CTGGCTGTCTCTCAATTTATA |
| 1231 | STAT1_031 | CCACACCATTGGTCTCGTGTT |
| 1232 | STAT1_032 | TGATCACTCTTTGCCACACCA |
| 1233 | STAT1_033 | TAGAACACGAGACCAATGGTG |
| 1234 | STAT1_034 | TCTTATTGTCAAGCATTAAAT |
| 1235 | STAT1_035 | ATGCTTGACAATAAGAGAAAG |
| 1236 | STAT1_036 | TGAACTACTTCCTAAAGGCAA |
| 1237 | STAT1_037 | ACTTTGTTTCTTCTATTGCCT |
| 1238 | STAT1_038 | TTTCTTCTATTGCCTTTAGGA |
| 1239 | STAT1_039 | TTCTATTGCCTTTAGGAAGTA |
| 1240 | STAT1_040 | GGAAGTAGTTCACAAAATAAT |
| 1241 | STAT1_041 | AGCTGCTGCCGAACTTGCTGC |
| 1242 | STAT1_042 | TGTTCCAATTCCTCCAACTTT |
| 1243 | STAT1_043 | TGATAGGGTCATGTTCGTAGG |
| 1244 | STAT1_044 | TTTTTTGTGATAGGGTCATGT |
| 1245 | STAT1_045 | CACCACAAACGAGCTGCAAAT |
| 1246 | STAT1_046 | CTGGGTATTTGCAGCTCGTTT |
| 1247 | STAT1_047 | CAGCTCGTTTGTGGTGGAAAG |
| 1248 | STAT1_048 | TGGTGGAAAGACAGCCCTGCA |
| 1249 | STAT1_049 | ACCAACAGTCTGGaaagaaaa |
| 1250 | STAT1_050 | tttttctttCCAGACTGTTGG |
| 1251 | STAT1_051 | tttCCAGACTGTTGGTGAAAT |
| 1252 | STAT1_052 | CAGACTGTTGGTGAAATTGCA |
| 1253 | STAT1_053 | AAATTATAATTCAGCTCTTGC |
| 1254 | STAT1_054 | ACTTTCAAATTATAATTCAGC |
| 1255 | STAT1_055 | AAAGTCAAAGTCTTATTTGAT |
| 1256 | STAT1_056 | TCTCATTCACATCTCTGCaaa |
| 1257 | STAT1_057 | CTGTATTTCTCTCATTCACAT |
| 1258 | STAT1_058 | CAGAGATGTGAATGAGAGAAA |
| 1259 | STAT1_059 | AGCATTTCTTTCCTATATTGT |
| 1260 | STAT1_060 | TTTCCTATATTGTATAGATTT |
| 1261 | STAT1_061 | CTATATTGTATAGATTTAGGA |
| 1262 | STAT1_062 | TGTGCGTGCCCAAAATGTTGA |
| 1263 | STAT1_063 | GGAAGTTCAACATTTTGGGCA |
| 1264 | STAT1_064 | GGCACGCACACAAAAGTGATG |
| 1265 | STAT1_065 | AATTGCTATAAAACAAATAAT |
| 1266 | STAT1_066 | CTAAGATGATTATTTGTTTTA |
| 1267 | STAT1_067 | TGTTCTTTCAATTGCTATAAA |
| 1268 | STAT1_068 | TTTTATAGCAATTGAAAGAAC |
| 1269 | STAT1_069 | TAGCAATTGAAAGAACAGAAA |
| 1270 | STAT1_070 | TTCTTTCCTTTTCTCTTCCAA |
| 1271 | STAT1_071 | CTTTTCTCTTCCAAGGGTCCT |
| 1272 | STAT1_072 | TCTTCCAAGGGTCCTCTCATC |
| 1273 | STAT1_073 | AAAACTAAGGGAGTGAAGCTC |
| 1274 | STAT1_074 | AAACCCAATTGTGCCAGCCTG |
| 1275 | STAT1_075 | AAGGTCTTTGTCATCCTTTAG |
| 1276 | STAT1_076 | TCATCCTTTAGACGACCTCTC |
| 1277 | STAT1_077 | GACGACCTCTCTGCCCGTTGT |
| 1278 | STAT1_078 | GTACAACATGCTGGTGGCGGA |
| 1279 | STAT1_079 | GGTGTTTTCTCTCTAGAATCT |
| 1280 | STAT1_080 | TCTCTAGAATCTGTCCTTCTT |
| 1281 | STAT1_081 | GTGACAGAAGAAAACTGCCAA |
| 1282 | STAT1_082 | AGAAGTGCTGAGTTGGCAGTT |
| 1283 | STAT1_083 | TTCTGTCACCAAAAGAGGTCT |
| 1284 | STAT1_084 | TTTGTTTAGGTCCTAACGCCA |
| 1285 | STAT1_085 | TTTAGGTCCTAACGCCAGCCC |
| 1286 | STAT1_086 | GGTCCTAACGCCAGCCCCGAT |
| 1287 | STAT1_087 | CTGAAAGTATACAAATGCAGA |
| 1288 | STAT1_088 | TATTTTCCTGAAAGTATACAA |
| 1289 | STAT1_089 | TCATTTATATTTTCCTGAAAG |
| 1290 | STAT1_090 | TATACTTTCAGGAAAATATAA |
| 1291 | STAT1_091 | AGGAAAATATAAATGATAAAA |
| 1292 | STAT1_092 | AATCCAAAGCCAGAAGGGAAA |
| 1293 | STAT1_093 | ATGAGTTCTAGGATGCTTTCA |
| 1294 | STAT1_094 | CCTTCTGGCTTTGGATTGAAA |
| 1295 | STAT1_095 | GATTGAAAGCATCCTAGAACT |
| 1296 | STAT1_096 | CTTAGCTTTTCTCCTTTTTAG |
| 1297 | STAT1_097 | TCCTTTTTAGAACCTGACTTC |
| 1298 | STAT1_098 | TTCGTGTAGGGTTCAACCGCA |
| 1299 | STAT1_099 | GAACCTGACTTCCATGCGGTT |
| 1300 | STAT1_100 | TAATTGCGAATGATGTCAGGG |
| 1301 | STAT1_101 | TGCTGTTACTTTCCCTGACAT |
| 1302 | STAT1_102 | CCTGACATCATTCGCAATTAC |
| 1303 | STAT1_103 | GATACAGATACTTCAGGGGAT |
| 1304 | STAT1_104 | TCAATATTTGGATACAGATAC |
| 1305 | STAT1_105 | CAAAGGCATGGTCTTTGTCAA |
| 1306 | STAT1_106 | GCCTGGAGTAATACTTTCCAA |
| 1307 | STAT1_107 | GAAAGTATTACTCCAGGCCAA |
| 1308 | STAT1_108 | GGGCCATCAAGTTCCATTGGC |
| 1309 | STAT1_109 | TTTCTAGCACCAGAGCCAATG |
| 1310 | STAT1_110 | TAGCACCAGAGCCAATGGAAC |
| 1311 | STAT1_111 | TTTTTCCCCATTTTAGTCACC |
| 1312 | STAT1_112 | CCCATTTTAGTCACCCTTCTA |
| 1313 | STAT1_113 | GTCACCCTTCTAGACTTCAGA |
| 1314 | STAT1_114 | ACGAGGTGTCTCGGATAGTGG |
| 1315 | STAT1_115 | TCTTTTTACAGATGAACACAG |
| 1316 | TWF1_001 | ACATCTTCACTTGCTGTGGAA |
| 1317 | TWF1_002 | CTTTCTTTATCTTTTTCCACA |
| 1318 | TWF1_003 | TTTATCTTTTTCCACAGCAAG |
| 1319 | TWF1_004 | TCTTTTTCCACAGCAAGTGAA |
| 1320 | TWF1_005 | CACAGCAAGTGAAGATGTTAA |
| 1321 | TWF1_006 | TGGCTCTGGCAAAGATCTCTT |
| 1322 | TWF1_007 | CATTTCTGGCTCTGGCAAAGA |
| 1323 | TWF1_008 | AGAAGTCTGTACTTTCCATTT |
| 1324 | TWF1_009 | CCAGAGCCAGAAATGGAAAGT |
| 1325 | TWF1_010 | AATAGATATTTTCAGAAGTCT |
| 1326 | TWF1_011 | TAAAAATAATTTTTCATAGAG |
| 1327 | TWF1_012 | ATAGAGCAACTTGTGATTGGA |
| 1328 | TWF1_013 | TCCTCCAACAGGGGTAAAACA |
| 1329 | TWF1_014 | TTTTACCCCTGTTGGAGGACA |
| 1330 | TWF1_015 | CCCCTGTTGGAGGACAAACAA |
| 1331 | TWF1_016 | ACGAACCTATTCAGTTGTGAA |
| 1332 | TWF1_017 | ACAACTGAATAGGTTCGTCAA |
| 1333 | TWF1_018 | ATGTGGCCACCTCCAAATTCC |
| 1334 | TWF1_019 | CTGTTCCAAATACTTCATCTT |
| 1335 | TWF1_020 | GAGGTGGCCACATTAAAGATG |
| 1336 | TWF1_021 | TATCCATGTAATGATACATCT |
| 1337 | TWF1_022 | ATCTGTCGTAGTTCTTCCTCA |
| 1338 | TWF1_023 | ATGCTTAGTGTCCACACCCAC |
| 1339 | TWF1_024 | CAAAGCCTGAAAGGCTTCTCG |
| 1340 | TWF1_025 | CCATTTCTCGAGAAGCCTTTC |
| 1341 | TWF1_026 | TCGAGAAGCCTTTCAGGCTTT |
| 1342 | TWF1_027 | AGGCTTTGGAAAAATTGAATA |
| 1343 | TWF1_028 | GAAAAATTGAATAATAGACAG |
| 1344 | TWF1_029 | CTGCCAATAAGAAACAAAATA |
| 1345 | TWF1_030 | TATCTATTTCCTGCCAATAAG |
| 1346 | TWF1_031 | ATTTTTTATATCTATTTCCTG |
| 1347 | TWF1_032 | TTTCTTATTGGCAGGAAATAG |
| 1348 | TWF1_033 | TTATTGGCAGGAAATAGATAT |
| 1349 | TWF1_034 | TTGTGTTGGCCAAAATTATAA |
| 1350 | TWF1_035 | AGTTCTGTATTTGTTGTGTTG |
| 1351 | TWF1_036 | GCAAATCTTTCAGTTCTGTAT |
| 1352 | TWF1_037 | GCCAACACAACAAATACAGAA |
| 1353 | TWF1_038 | CCAAAGAGGATTCCCAAGGAT |
| 1354 | TWF1_039 | TACAGAAAGAAATGGTAACGA |
| 1355 | TWF1_040 | TTTCTGTATAAACATTCCCAT |
| 1356 | TWF1_041 | TGTATAAACATTCCCATGAAG |
| 1357 | TWF1_042 | TTATTATTTGAACTTACAGTT |
| 1358 | TWF1_043 | AACTTACAGTTTTTATTTATT |
| 1359 | TWF1_044 | TTTATTCAATGCCTGGATACA |
| 1360 | TWF1_045 | TTCAATGCCTGGATACACATG |
| 1361 | TWF1_046 | TAGCAGACGGCTCTTGCAGCT |
| 1362 | TWF1_047 | TACAATTTCTAGCAGACGGCT |
| 1363 | TWF1_048 | TAGTTGTCTTTCTACAATTTC |
| 1364 | TWF1_049 | TAATTACATCCATTTGTAGTT |
| 1365 | TWF1_050 | TCTTTTACAGATCGAGATAGA |
| 1366 | TWF1_051 | CAGATCGAGATAGACAATGGG |
| 1367 | TWF1_052 | CTTGTGTGCATGCTGCTTGGG |
| 1368 | TWF1_053 | TGAAGAAGTACATCCCAAGCA |
| 1369 | TWF1_054 | CAAAACTTTGCTTGTGTGCAT |
| 1370 | TWF1_055 | GTTTTGCAAAACTTTGCTTGT |
| 1371 | TWF1_056 | CTGCAGGACCTTTTGGTTTTG |
| 1372 | TWF1_057 | CAAAACCAAAAGGTCCTGCAG |
| 1373 | TWF1_058 | CGCTGGGCCCCTAATTAGTCT |
| 1374 | TWF1_059 | ATCAGTAGTAGCTTCAGTTTC |
| 1375 | TWF1_060 | ATGTGATGACTTTAATCAGTA |
| 1376 | TWF1_061 | AAAAACTAGTATTACAATGTT |
| 1377 | TWF1_062 | AGTTCTCCTGTACTAAAAGCT |
| 1378 | TWF1_063 | AAAGTCCAGCTTTTAGTACAG |
| 1379 | TWF1_064 | TATCAACATGGAATGATTTCA |
| 1380 | TWF1_065 | GTACAGGAGAACTGAAATCAT |
| 1381 | TWF1_066 | CCTACTTTATATCAACATGGA |
| 1382 | TWF1_067 | CAAAAAGTACAATTTTTTTCC |
| 1383 | TWF1_068 | AAAACACACAGAAGTGAAAAG |
| 1384 | TWF1_069 | GAAAATAGCACTTTTCACTTC |
| 1385 | TWF1_070 | ACTTCTGTGTGTTTTTAAAAT |
| 1386 | TWF1_071 | AAATTAATGTTATAGAAGACT |
| 1387 | TWF1_072 | ACTCAAAAATAGAAATCATGA |
| 1388 | TWF1_073 | TAGCTTTAACTCAAAAATAGA |
| 1389 | TWF1_074 | TATTTTTGAGTTAAAGCTAGA |
| 1390 | TWF1_075 | AGTTAAAGCTAGAAAAGGGTT |
| 1391 | TWF1_076 | ATTTTGTCACACTGTTTTCAT |
| 1392 | TWF1_077 | AAGTGTGGAATCAACGCTATG |
| 1393 | TWF1_078 | TCACACTGTTTTCATAGCGTT |
| 1394 | TWF1_079 | AGAAGTATTTGAAGTGTGGAA |
| 1395 | TWF1_080 | ATAGCGTTGATTCCACACTTC |
| 1396 | TWF1_081 | TAGAACTGGCCCAACTGTATA |
| 1397 | TWF1_082 | AGACATCAGACTTTCTAGAAC |
| 1398 | TWF1_083 | TACAGTTGGGCCAGTTCTAGA |
| 1399 | TWF1_084 | CCCTTTGAGACATCAGACTTT |
| 1400 | TWF1_085 | TGTCCCACAAGAAAGTAGTAA |
| 1401 | TWF1_086 | AGGTCTTTCTGTCCCACAAGA |
| 1402 | TWF1_087 | TTGTGGGACAGAAAGACCTTA |
| 1403 | TWF1_088 | CAGCTTAGAAAATACTCTAGC |
| 1404 | TWF1_089 | CAAGGCACACTAAGTTTCCAG |
| 1405 | TWF1_090 | TAAGCTGGAAACTTAGTGTGC |
| 1406 | TWF1_091 | TAAAAGTTGCAGACATGATCC |
| 1407 | TWF1_092 | GAAATAGTGCTTTATATTGCA |
| 1408 | TWF1_093 | TATTGCAGCAGTCTTTTATAT |
| 1409 | TWF1_094 | ATGCTATTaaaaaaaaGTCAA |
| 1410 | TWF1_095 | TATTTGACttttttttAATAG |
| 1411 | TWF1_096 | ACttttttttAATAGCATTAA |
| 1412 | TWF1_097 | AGAGTGAGCTGATCTGCAATT |
| 1413 | TWF1_098 | ATAGCATTAAAATTGCAGATC |
| 1414 | TWF1_099 | AGGGTACCAGATATTTTCTAT |
| 1415 | TWF1_100 | AATGTCATCAGAAATCCTGCA |
| 1416 | TWF1_101 | AAATAGGTGGGCTACCTTTCT |
| 1417 | ERAP1_001 | AGGGGCAGAAACACCATCTTC |
| 1418 | ERAP1_002 | TGCCCCTCAAATGGTCCCTTG |
| 1419 | ERAP1_003 | TACTTTCCTCACTGTTGGCTC |
| 1420 | ERAP1_004 | CTCACTGTTGGCTCTCTTAAC |
| 1421 | ERAP1_005 | GAGATGCTTCAGTGCTCTGAC |
| 1422 | ERAP1_006 | TTCCAAGGAAATGGTGTCCCA |
| 1423 | ERAP1_007 | CTTGGAATAAAATACGACTTC |
| 1424 | ERAP1_008 | CATGGATCAAGAGATCATAAT |
| 1425 | ERAP1_009 | GTGGTTCCCCAGAAGGTCAGC |
| 1426 | ERAP1_010 | TACTTTCGTGGTTCCCCAGAA |
| 1427 | ERAP1_011 | CTCCTGACGGGGGTGTTCCAG |
| 1428 | ERAP1_012 | TAAAATCCGTGGAAAGTCTCC |
| 1429 | ERAP1_013 | GGAGACTTTCCACGGATTTTA |
| 1430 | ERAP1_014 | CACGGATTTTACAAAAGCACC |
| 1431 | ERAP1_015 | CAAAAGCACCTACAGAACCAA |
| 1432 | ERAP1_016 | TTTCACTTGCCTTAATTTTAG |
| 1433 | ERAP1_017 | ACTTGCCTTAATTTTAGGATA |
| 1434 | ERAP1_018 | GGATACTAGCATCAACACAAT |
| 1435 | ERAP1_019 | AACCCACTGCAGCTAGAATGG |
| 1436 | ERAP1_020 | AAGGCAGGTTCATCAAAGCAG |
| 1437 | ERAP1_021 | CCTGCTTTGATGAACCTGCCT |
| 1438 | ERAP1_022 | ATTGAGAAACTTGCTTTGAAG |
| 1439 | ERAP1_023 | ATGAACCTGCCTTCAAAGCAA |
| 1440 | ERAP1_024 | TCAATCAAAATTAGAAGAGAG |
| 1441 | ERAP1_025 | AATTATTCTGATTTTAGGTGA |
| 1442 | ERAP1_026 | GGTGAAATCTGTGACTGTTGC |
| 1443 | ERAP1_027 | ATGTCACTGTGAAGATGAGCA |
| 1444 | ERAP1_028 | AGATTTTGAGTCTGTCAGCAA |
| 1445 | ERAP1_029 | AGTCTGTCAGCAAGATAACCA |
| 1446 | ERAP1_030 | TCTTGTCTGGCACAGCATAAA |
| 1447 | ERAP1_031 | TCTTTAGGTTTCTGTTTATGC |
| 1448 | ERAP1_032 | GGTTTCTGTTTATGCTGTGCC |
| 1449 | ERAP1_033 | TGTTTATGCTGTGCCAGACAA |
| 1450 | ERAP1_034 | TGCTGTGCCAGACAAGATAAA |
| 1451 | ERAP1_035 | GGTAGGGGATACGGTATGCTG |
| 1452 | ERAP1_036 | TGAGGATTATTTCAGCATACC |
| 1453 | ERAP1_037 | AGCATACCGTATCCCCTACCC |
| 1454 | ERAP1_038 | TTACTTCCTTCCCAAGATCTT |
| 1455 | ERAP1_039 | CATAGCACCAGACTGAAAGTC |
| 1456 | ERAP1_040 | AGTCTGGTGCTATGGAAAACT |
| 1457 | ERAP1_041 | TGCATCAAACAACAGAGCAGA |
| 1458 | ERAP1_042 | ATGCAGAAAAGTCTTCTGCAT |
| 1459 | ERAP1_043 | GTGGTTTGGGAACCTGGTCAC |
| 1460 | ERAP1_044 | GGAACCTGGTCACTATGGAAT |
| 1461 | ERAP1_045 | GCCAAAGATCATTCCACCATT |
| 1462 | ERAP1_046 | GCAAATCCTTCATTTAGCCAA |
| 1463 | ERAP1_047 | GCTAAATGAAGGATTTGCCAA |
| 1464 | ERAP1_048 | CCAAATTTATGGAGTTTGTGT |
| 1465 | ERAP1_049 | AGTTCAGGATGGGTCACACTG |
| 1466 | ERAP1_050 | TGGAGTTTGTGTCTGTCAGTG |
| 1467 | ERAP1_051 | TGTCTGTCAGTGTGACCCATC |
| 1468 | ERAP1_052 | CCAAAGAAATAATCTCCCTAT |
| 1469 | ERAP1_053 | TTTTTTCTATCTCAATAGGGA |
| 1470 | ERAP1_054 | TATCTCAATAGGGAGATTATT |
| 1471 | ERAP1_055 | AAGCATCTACCTCCATTGCGT |
| 1472 | ERAP1_056 | TTTGGCAAATGTTTTGACGCA |
| 1473 | ERAP1_057 | GCAAATGTTTTGACGCAATGG |
| 1474 | ERAP1_058 | ACGCAATGGAGGTAGATGCTT |
| 1475 | ERAP1_059 | CACAGGTGTAGACACAGGGTG |
| 1476 | ERAP1_060 | AATTCCTCACACCCTGTGTCT |
| 1477 | ERAP1_061 | CCTTATCATAAGAAACATCAT |
| 1478 | ERAP1_062 | ATGATGTTTCTTATGATAAGG |
| 1479 | ERAP1_063 | TATTATCTTTTCAGGGAGCTT |
| 1480 | ERAP1_064 | AGGGAGCTTGTATTCTGAATA |
| 1481 | ERAP1_065 | AATGCGTCAGCACTAAGATAC |
| 1482 | ERAP1_066 | TAGCTATGCTTCTGGAGATAC |
| 1483 | ERAP1_067 | AAAGTGGTATTGTACAGTATC |
| 1484 | ERAP1_068 | TATTTTTATAGCTATGCTTCT |
| 1485 | ERAP1_069 | CACCATCTGTAGGGCAAATCT |
| 1486 | ERAP1_070 | TTTTTGGTTTTTAGATTTGCC |
| 1487 | ERAP1_071 | GTTTTTAGATTTGCCCTACAG |
| 1488 | ERAP1_072 | GATTTGCCCTACAGATGGTGT |
| 1489 | ERAP1_073 | CCCTACAGATGGTGTAAAAGG |
| 1490 | ERAP1_074 | CTCTAGAAGTCAACATTCATC |
| 1491 | ERAP1_075 | TGTACACACCAGCATTGGCAT |
| 1492 | ERAP1_076 | ACATCCACCCCTTCCTGATGC |
| 1493 | ERAP1_077 | CCCTAATAACCATCACAGTGA |
| 1494 | ERAP1_078 | CTCTAGGAGCATTACCCAGTG |
| 1495 | ERAP1_079 | TTTTAGGTACCTGTGGCATGT |
| 1496 | ERAP1_080 | CTGGTGATGAATGTCAATGGA |
| 1497 | ERAP1_081 | GGTACCTGTGGCATGTTCCAT |
| 1498 | ERAP1_082 | GCAAAAATCGATGGACCATGT |
| 1499 | ERAP1_083 | TTTTTAGCAAAAATCGATGGA |
| 1500 | ERAP1_084 | CAAAATAAATTACCTGTTTTT |
| 1501 | ERAP1_085 | ATTTATTTTATTTACTCTAGA |
| 1502 | ERAP1_086 | TTTTATTTACTCTAGATGTGC |
| 1503 | ERAP1_087 | TTTACTCTAGATGTGCTCATC |
| 1504 | ERAP1_088 | CTCTAGATGTGCTCATCCTCC |
| 1505 | ERAP1_089 | ATCCATTCCACCTCTTCTGGG |
| 1506 | ERAP1_090 | ATGTGGGCATGAATGGCTATT |
| 1507 | ERAP1_091 | AAAGGCCAGTCAAAGAGTCCC |
| 1508 | ERAP1_092 | ACTGGCCTTTTAAAAGGAACA |
| 1509 | ERAP1_093 | AAAGGAACACACACAGCAGTC |
| 1510 | ERAP1_094 | TGCAGCGTGTATTACCTGACG |
| 1511 | ERAP1_095 | AAGTACAGGGATAAATCCAAG |
| 1512 | ERAP1_096 | ATGTTTCAAGTACAGGGATAA |
| 1513 | ERAP1_097 | AGTTTCATGTTTCAAGTACAG |
| 1514 | ERAP1_098 | TCCCTGTACTTGAAACATGAA |
| 1515 | ERAP1_099 | AAGGTTTGAATGAGCTGATTC |
| 1516 | ERAP1_100 | TCCATTAACTTATACATAGGA |
| 1517 | ERAP1_101 | AATGAGCTGATTCCTATGTAT |
| 1518 | ERAP1_102 | CACTTCATTCATATCTCTTTT |
| 1519 | ERAP1_103 | CCTTGAATTGAGTTTCCACTT |
| 1520 | ERAP1_104 | AGGCTTTTACCTTGAATTGAG |
| 1521 | ERAP1_105 | TTTCAGGCTTTTACCTTGAAT |
| 1522 | ERAP1_106 | CCTGTACAACGCCCTCAGGCC |
| 1523 | ERAP1_107 | TGAAATAGCCTTCTGCCCTCT |
| 1524 | ERAP1_108 | CATTGGATTCCTTCCACTTTC |
| 1525 | ERAP1_109 | AGAAAGTGGAAGGAATCCAAT |
| 1526 | ERAP1_110 | GTAAGGACTGACCTCAAGTTT |
| 1527 | ERAP1_111 | TTTTCTTAATCCTTCTAGGCT |
| 1528 | ERAP1_112 | TTAATCCTTCTAGGCTACTAG |
| 1529 | ERAP1_113 | TCTCCCTTAAAGCTTTCATCT |
| 1530 | ERAP1_114 | TTTTATCTCCCTTAAAGCTTT |
| 1531 | ERAP1_115 | TGGAAACTCCTGAGTTTTTAT |
| 1532 | ERAP1_116 | AGGGAGATAAAATAAAAACTC |
| 1533 | ERAP1_117 | CACAAATTCTTACACTCATTG |
| 1534 | ERAP1_118 | CTCAGAAATTGCCAGGCCAGT |
| 1535 | ERAP1_119 | TTCCAGTTTTTCCTCAGAAAT |
| 1536 | ERAP1_120 | TACAAGTTTGTTCCAGTTTTT |
| 1537 | ERAP1_121 | TGAGGAAAAACTGGAACAAAC |
| 1538 | ERAP1_122 | GCACCACTTACTTTTGTACAA |
| 1539 | ERAP1_123 | AATATTTCCCTCTCTAGGTTT |
| 1540 | ERAP1_124 | CCTCTCTAGGTTTGAACTTGG |
| 1541 | ERAP1_125 | AACTTGGCTCATCTTCCATAG |
| 1542 | ERAP1_126 | TTGTACCCATTACCATGTGGG |
| 1543 | ERAP1_127 | CCTCTTCAAGCCGTGTTCTTG |
| 1544 | ERAP1_128 | AAAGAGCTGAAGAATCCTTTT |
| 1545 | ERAP1__129 | TTTCAAAGAGCTGAAGAATCC |
| 1546 | ERAP1_130 | CTCACAAGGTAAAAGGATTCT |
| 1547 | ERAP1_131 | AAAGAAAATGGTTCTCAGCTC |
| 1548 | ERAP1_132 | AATTGTCTGTTGGACACAACG |
| 1549 | ERAP1_133 | TTCAATGGTTTCAATTGTCTG |
| 1550 | ERAP1_134 | TCAAAATTCTTATCCATCCAA |
| 1551 | ERAP1_135 | CAGCCACACTCTGATTTTATC |
| 1552 | ERAP1_136 | ACTTTGCAGCCACACTCTGAT |
| 1553 | ERAP1_137 | ATAAAATCAGAGTGTGGCTGC |
| 1554 | ERAP1_138 | CATACGTTCAAGCTTTTCACT |
| 1555 | IFNGR1_001 | TCCTACCCCTTGTCATGCAGG |
| 1556 | IFNGR1_002 | CTTTTTTATTTTCTTACAGTG |
| 1557 | IFNGR1_003 | TTTTCTTACAGTGCCTACACC |
| 1558 | IFNGR1_004 | TTACAGTGCCTACACCAACTA |
| 1559 | IFNGR1_005 | CCTCTACGGTAAAAACAGGGA |
| 1560 | IFNGR1_006 | CCGTAGAGGTAAAGAACTATG |
| 1561 | IFNGR1_007 | TTCTTTTTAGTGTTAAGAATT |
| 1562 | IFNGR1_008 | GTGTTAAGAATTCAGAATGGA |
| 1563 | IFNGR1_009 | TCATCATTATTGTAATATTTC |
| 1564 | IFNGR1_010 | ATGGATCACCAACATGATCAG |
| 1565 | IFNGR1_011 | TGATCATGTTGGTGATCCATC |
| 1566 | IFNGR1_012 | ACTCTGACCCAAAGAGAATTT |
| 1567 | IFNGR1_013 | TCCAACCCTGGCTTTAACTCT |
| 1568 | IFNGR1_014 | GGTCAGAGTTAAAGCCAGGGT |
| 1569 | IFNGR1_015 | CATAGGCAGATTCTTTTTGTC |
| 1570 | IFNGR1_016 | GGTGGTCCAATTTTTCCTGGG |
| 1571 | IFNGR1_017 | TGATATCCAGTTTAGGTGGTC |
| 1572 | IFNGR1_018 | CTTCTCCTCCTTTCTGATATC |
| 1573 | IFNGR1_019 | CAAAAACTGAAGGGTGAAATA |
| 1574 | IFNGR1_020 | ACCCTTCAGTTTTTGTAAATG |
| 1575 | IFNGR1_021 | GGGATCATAATCGACTTCCTG |
| 1576 | IFNGR1_022 | TAAATGGAGACGAGCAGGAAG |
| 1577 | IFNGR1_023 | TTTTTTCATCTAGATCCAGTA |
| 1578 | IFNGR1_024 | ATCTAGATCCAGTATAAAATA |
| 1579 | IFNGR1_025 | AGTTGTAACACCCCACACATG |
| 1580 | IFNGR1_026 | AGCAGAAGGAGTCTTACATGT |
| 1581 | IFNGR1_027 | ACTTTTCAGTTGTAACACCCC |
| 1582 | IFNGR1_028 | TACTGCTATTGAAAATGGTAA |
| 1583 | IFNGR1_029 | TATTACCATTTTCAATAGCAG |
| 1584 | IFNGR1_030 | TGCCTTTTTTAAGGTTCTCTT |
| 1585 | IFNGR1_031 | AGGTTCTCTTTGGATTCCAGT |
| 1586 | IFNGR1_032 | GATTCCAGTTGTTGCTGCTTT |
| 1587 | IFNGR1_033 | CTACTCTTTCTAGTGCTTAGC |
| 1588 | IFNGR1_034 | TTAATATAAAAACAGATGAAT |
| 1589 | IFNGR1_035 | TAGTGCTTAGCCTGGTATTCA |
| 1590 | IFNGR1_036 | CTTCAATGGATTAATTTTCTT |
| 1591 | IFNGR1_037 | TATTAAGAAAATTAATCCATT |
| 1592 | IFNGR1_038 | ATCAATTTTTCTCCCCATAGA |
| 1593 | IFNGR1_039 | TCCCCATAGATCTCTGTGGTA |
| 1594 | IFNGR1_040 | TCTCTAAAGTAGCACTTCTTA |
| 1595 | IFNGR1_041 | ATTCAGGTTTTGTCTCTAAAG |
| 1596 | IFNGR1_042 | GAGACAAAACCTGAATCAAAA |
| 1597 | IFNGR1_043 | TAAGGAAAATGGCTGGTATGA |
| 1598 | IFNGR1_044 | CTTAGAAAAGGAGGTGGTCTG |
| 1599 | IFNGR1_045 | CTGGATTGTCTTCGGTATGCA |
| 1600 | IFNGR1_046 | TTCAGTAGTCACCACTTCTGT |
| 1601 | IFNGR1_047 | TAGTATAACAGAAGTGGTGAC |
| 1602 | IFNGR1_048 | AAGCGATGCTGCCAGGTTCAG |
| 1603 | IFNGR1_049 | AGTAGTAACCAGTCTGAACCT |
| 1604 | IFNGR1_050 | TGGAGTGATACGAGTTTAAAG |
| 1605 | IFNGR1_051 | AACTCGTATCACTCCAGAAAT |
| 1606 | IFNGR1_052 | TGGAGTGATCACTCTCAGAAC |
| 1607 | IFNGR1_053 | ATACTGATTCCAGCTGTCTGG |
| 1608 | IFNGR1_054 | GGGGAAATTCTGAGTCAGATA |
| 1609 | IFNGR1_055 | TTATTTGGGGGAAATTCTGAG |
| 1610 | IFNGR1_056 | ACCTTTATTATTTGGGGGAAA |
| 1611 | IFNGR1_057 | TTTCACCTTTATTATTTGGGG |
| 1612 | IFNGR1_058 | CCCCAAATAATAAAGGTGAAA |
| 1613 | IFNGR1_059 | TTACGGTTATGAGCTCTTGTC |
| 1614 | IFNGR1_060 | TCATAACCAAAGGAGGTGGGG |
| 1615 | IFNGR1_061 | GTTATGATAAACCACATGTGC |
| 1616 | IFNGR1_062 | CCGCTATCATCCACAAGTAGA |
| 1617 | IFNGR1_063 | GAATCTTCTGTTGGTCTATAA |
| 1618 | IFNGR2_001 | tctgtccccctcaagaccctc |
| 1619 | IFNGR2_002 | CCAGCTGCCCGCTCCTCAGCA |
| 1620 | IFNGR2_003 | AACTGCACTTGGTAGACAACA |
| 1621 | IFNGR2_004 | AATAGTAAGCCGGTATTTCTG |
| 1622 | IFNGR2_005 | CTTCCCAGCACCGACAGTAAA |
| 1623 | IFNGR2_006 | AATGTCACTCTACGCCTTCGA |
| 1624 | IFNGR2_007 | TGGAGGCCCGACAGTCACTGA |
| 1625 | IFNGR2_008 | TCTTTGTAATTCTTTTTCAGT |
| 1626 | IFNGR2_009 | TAATTCTTTTTCAGTGACTGT |
| 1627 | IFNGR2_010 | AGTGACTGTCGGGCCTCCAGA |
| 1628 | IFNGR2_011 | ACATCGCTGATACCTCCACGG |
| 1629 | IFNGR2_012 | CCAGTAATGGACATAATAACA |
| 1630 | IFNGR2_013 | TTATTATGTCCATTACTGGGA |
| 1631 | IFNGR2_014 | AAACAGGTCAAAGGCCCTTTC |
| 1632 | IFNGR2_015 | AGTTATCCAATGAAATGGAGT |
| 1633 | IFNGR2_016 | AGAAGCAACTCCATTTCATTG |
| 1634 | IFNGR2_017 | ATTGGATAACTTAAAACCCTC |
| 1635 | IFNGR2_018 | TTCCAAAGCAGTTGTGCCTGG |
| 1636 | IFNGR2_019 | CAAGTCCAGGCACAACTGCTT |
| 1637 | IFNGR2_020 | GAACAAAAGTAACATCTTTAG |
| 1638 | IFNGR2_021 | GTAGCAAGATATGTTGCTTAA |
| 1639 | IFNGR2_022 | GAGTCGGGCATTTAAGCAACA |
| 1640 | IFNGR2_023 | CCATCTGCCATTGTTTCGTAG |
| 1641 | IFNGR2_024 | AGCAACATATCTTGCTACGAA |
| 1642 | IFNGR2_025 | GTGTCCTCTTTTTAGCCTCCA |
| 1643 | IFNGR2_026 | GCCTCCACTGAGCTTCAGCAA |
| 1644 | IFNGR2_027 | GTTGCTGTCGGTGCTGGCAGG |
| 1645 | IFNGR2_028 | AGGACCAGGAAGAAACAGGCT |
| 1646 | IFNGR2_029 | ATCAGGCCTCTATATTTCAGG |
| 1647 | IFNGR2_030 | TTCCTGGTCCTGAAATATAGA |
| 1648 | IFNGR2_031 | ACACTCCACCAAGCATCCCAT |
| 1649 | IFNGR2_032 | CTTTCCAACCTCCTCAAGTAT |
| 1650 | IFNGR2_033 | CAACCTCCTCAAGTATTTAAA |
| 1651 | IFNGR2_034 | AAAGACCCAACTCAGCCCATC |
| 1652 | IFNGR2_035 | GTGAGCTGTCCTTGTCCAAGG |
| 1653 | IFNGR2_036 | CGGAAACGAGATAATGGACAC |
| 1654 | IFNGR2_037 | GAGAACATCTTCTTGCTCCTT |
| 1655 | IFNGR2_038 | CGGAAAAGGAGCAAGAAGATG |
| 1656 | IFNGR2_039 | GTTCAAAGCGTTTGGAGAACA |
| 1657 | JAK1_001 | AAAATATGCAAATCTACATAC |
| 1658 | JAK1_002 | CTTCCACAACAGTATCTAAAT |
| 1659 | JAK1_003 | GCACAGAAAGCCATGGCATTG |
| 1660 | JAK1_004 | TGTGCTAAAATGAGGAGCTCC |
| 1661 | JAK1_005 | CTTTTCCTCAGGTATCTCTCC |
| 1662 | JAK1_006 | CTCAGGTATCTCTCCTCTTTG |
| 1663 | JAK1_007 | TCACAACCTCTTTGCCCTGTA |
| 1664 | JAK1_008 | GAGCATACCAGAGCTTGGTGT |
| 1665 | JAK1_009 | CCCTGTATGACGAGAACACCA |
| 1666 | JAK1_010 | CTGCCTTCCAGGTTCTATTTC |
| 1667 | JAK1_011 | ACCAATTGGCATGGAACCAAC |
| 1668 | JAK1_012 | GAGAATGACGCCACACTGACT |
| 1669 | JAK1_013 | TGCTTCTTTGGAGAATGACGC |
| 1670 | JAK1_014 | TCGTAGCCATTTTTCTGCTTC |
| 1671 | JAK1_015 | ACCAAATCATACTGTCCCTAG |
| 1672 | JAK1_016 | TCCCCCTTGCTCCTAGGGACA |
| 1673 | JAK1_017 | GTGAAATGCCTGGCTCCTATT |
| 1674 | JAK1_018 | CCTGATGTCCTTGGGCAGTTC |
| 1675 | JAK1_019 | TGGAATATATCGCTTGTAGCT |
| 1676 | JAK1_020 | TACTGTCTTTTAGCTACAAGC |
| 1677 | JAK1_021 | GCTACAAGCGATATATTCCAG |
| 1678 | JAK1_022 | TCCGCATCCTGGTGAGAAGGT |
| 1679 | JAK1_023 | GGAAATCCTTGAAAACATTAT |
| 1680 | JAK1_024 | AAGGATTTCCTAAAGGAATTT |
| 1681 | JAK1_025 | CTAAAGGAATTTAACAACAAG |
| 1682 | JAK1_026 | ACAACAAGACCATTTGTGACA |
| 1683 | JAK1_027 | ACCTTCAGGTCATGCGTGGAC |
| 1684 | JAK1_028 | TGACAGCAGCGTGTCCACGCA |
| 1685 | JAK1_029 | CAAGGTAGCCAAGTATTTCAC |
| 1686 | JAK1_030 | TCAAAGTTTCCAAGGTAGCCA |
| 1687 | JAK1_031 | AGCACCGTAATGTTTTGTCAA |
| 1688 | JAK1_032 | ACAAAACATTACGGTGCTGAA |
| 1689 | JAK1_033 | TGATGAAATCAGTAACATGGA |
| 1690 | JAK1_034 | AGACTTCCATGTTACTGATTT |
| 1691 | JAK1_035 | ATCAGAAAATGAGATGAATTG |
| 1692 | JAK1_036 | CACCGTCATTCGAATGAAACC |
| 1693 | JAK1_037 | ATTCGAATGACGGTGGAAACG |
| 1694 | JAK1_038 | TGCCTCCACTGGATTCCAAGA |
| 1695 | JAK1_039 | GTTTATGCCTCCACTGGATTC |
| 1696 | JAK1_040 | cttttcAACAGAAACAACCTG |
| 1697 | JAK1_041 | TGTATCTTATCAGGTTGTTTC |
| 1698 | JAK1_042 | tttttttccttttcAACAGAA |
| 1699 | JAK1_043 | cgcttcagtttatttttttcc |
| 1700 | JAK1_044 | TGTTgaaaaggaaaaaaataa |
| 1701 | JAK1_045 | cagttttttccgcttcagttt |
| 1702 | JAK1_046 | ttttccagttttttccgcttc |
| 1703 | JAK1_047 | TCCTCATCCTTCTTGTgttta |
| 1704 | JAK1_048 | AGGGAAGTAAGAAAAATTGTT |
| 1705 | JAK1_049 | TTACAATGTGAGTGATTTCAG |
| 1706 | JAK1_050 | TTACTTCCCTGAAATCACTCA |
| 1707 | JAK1_051 | TTGTTGTCCTGCTTGTTAATG |
| 1708 | JAK1_052 | TTCTCTCTCAACAGGAACTGA |
| 1709 | JAK1_053 | TGTCCCTGGTAGATGGCTACT |
| 1710 | JAK1_054 | TTGATGGCGTATTCTGTACTA |
| 1711 | JAK1_055 | CCTACTTCTCCCTCTAGTACA |
| 1712 | JAK1_056 | ACAACATCCTCATGACCGTCA |
| 1713 | JAK1_057 | CCGAATAGCAGGTGCAGGGTG |
| 1714 | JAK1_058 | AGATCGAGGTGCAGAAGGGCC |
| 1715 | JAK1_059 | GCATGAAGCTGATGTTATCCG |
| 1716 | JAK1_060 | TTAGTAGCCACCAGCAGGTTG |
| 1717 | JAK1_061 | GATCGGATCCTCAAGAAGGAT |
| 1718 | JAK1_062 | TCTTCTTCTCTTCAGAAGTTC |
| 1719 | JAK1_063 | AGGATCACTTTTATCTTCTTC |
| 1720 | JAK1_064 | TGGGAGACCTGTCTCATCATG |
| 1721 | JAK1_065 | AAAGAGAACACACTTACTCTC |
| 1722 | JAK1_066 | TGCCTACAGATATCATGGTGG |
| 1723 | JAK1_067 | CGGTGCATGAAGAGATCCAGA |
| 1724 | JAK1_068 | TGGAAGGGGGTCCTCTGGATC |
| 1725 | JAK1_069 | CATGGTGTGGTAAGGACATCG |
| 1726 | JAK1_070 | AATTTCCATGGTGTGGTAAGG |
| 1727 | JAK1_071 | GCAACTTTGAATTTCCATGGT |
| 1728 | JAK1_072 | CATGGACCAGGTCTTTATCCT |
| 1729 | JAK1_073 | CTCTGCAGGAGGATAAAGACC |
| 1730 | JAK1_074 | GTACACACATTTCCATGGACC |
| 1731 | JAK1_075 | CCAGAGCGTGGTTCCAAAGCT |
| 1732 | JAK1_076 | GAACCACGCTCTGGGAAATCT |
| 1733 | JAK1_077 | AAGGGGATCTCGCCATTGTAG |
| 1734 | JAK1_078 | CAGAAAGAGAGATTCTATGAA |
| 1735 | JAK1_079 | TTCCGAGCCATCATGAGAGAC |
| 1736 | JAK1_080 | TGAAACAATATCTGGATCTAA |
| 1737 | JAK1_081 | TTTTCTCTTCTGTTAGATCCA |
| 1738 | JAK1_082 | TCTTCTGTTAGATCCAGATAT |
| 1739 | JAK1_083 | AGaaaaaaaaCCAGCAACTGA |
| 1740 | JAK1_084 | AAAATGTGTGGGGTCCACTTC |
| 1741 | JAK1_085 | GGAAGCGCTTTTCAAAATGTG |
| 1742 | JAK1_086 | AAAAGCGCTTCCTAAAGAGGA |
| 1743 | JAK1_087 | CCTCCAGGGCCACTTTGGGAA |
| 1744 | JAK1_088 | GGAAGGTTGAGCTCTGCAGGT |
| 1745 | JAK1_089 | ACAGCCACCTGCTCCCCTGTA |
| 1746 | JAK1_090 | AGATCAGCTATGTGGTTACCT |
| 1747 | JAK1_091 | CTTTTTCAGATCAGCTATGTG |
| 1748 | JAK1_092 | TACTTCACAATGTTCTCATGA |
| 1749 | JAK1_093 | TAAACAGGAGGAAATGGTATT |
| 1750 | JAK1_094 | GAAGATATTCCTTAAGGCTTC |
| 1751 | JAK1_095 | TGCCTTCGGGAAGCCTTAAGG |
| 1752 | JAK1_096 | TTCTTATTCTTTGGAAGATAT |
| 1753 | JAK1_097 | TTTTGTTCTTATTCTTTGGAA |
| 1754 | JAK1_098 | AGGTTTATTTTGTTCTTATTC |
| 1755 | JAK1_099 | GCTGCTGTTTGAGGTTTATTT |
| 1756 | JAK1_100 | CCTTACAAATCTGAACGGCAT |
| 1757 | JAK1_101 | TTTTTTACCTTACAAATCTGA |
| 1758 | JAK1_102 | CTTCTCTCTCTCAGGGGATGG |
| 1759 | JAK1_103 | TTGCTGCCAAGTCCCGGTGAA |
| 1760 | JAK1_104 | GGTTCTCGGCAATACGTTCAC |
| 1761 | JAK1_105 | ACTTGGTGTTCACTCTCAACA |
| 1762 | JAK1_106 | GTTAAACCGAAGTCTCCAATT |
| 1763 | JAK1_107 | AATTGCTTTGGTTAAACCGAA |
| 1764 | JAK1_108 | ACCAAAGCAATTGAAACCGAT |
| 1765 | JAK1_109 | ATTCAGTTACCAAAACACAGG |
| 1766 | JAK1_110 | TGTTCTGCTTCCTTTCAAGGT |
| 1767 | JAK1_111 | GATTGCATTAAACATTCTGGA |
| 1768 | JAK1_112 | AAGGTATGCTCCAGAATGTTT |
| 1769 | JAK1_113 | ATGCAATCTAAATTTTATATT |
| 1770 | JAK1_114 | TATTGCCTCTGACGTCTGGTC |
| 1771 | JAK1_115 | GAGTCACTCTGCATGAGCTGC |
| 1772 | JAK1_116 | TTTGATTTTATTTTATATAGT |
| 1773 | JAK1_117 | ATTTTATTTTATATAGTTGTT |
| 1774 | JAK1_118 | TTTTATATAGTTGTTCCTGAA |
| 1775 | JAK1_119 | TATAGTTGTTCCTGAAAATGA |
| 1776 | JAK1_120 | ACGTATTCACAAGTCTTGTGA |
| 1777 | JAK1_121 | CTTCTTTTAACGTATTCACAA |
| 1778 | JAK1_122 | CTCATAAGTTGATAAACCTGT |
| 1779 | JAK1_123 | TTTTTACAGGTTTATCAACTT |
| 1780 | JAK1_124 | CAGGTTTATCAACTTATGAGG |
| 1781 | JAK1_125 | TCAACTTATGAGGAAATGCTG |
| 1782 | JAK1_126 | AAAGTGCTTCAAATCCTTCAA |
| 1783 | JAK1_127 | AGAACCTTATTGAAGGATTTG |
| 1784 | JAK1_128 | AATGTTATTCATGCTTCTTAT |
| 1785 | JAK2_001 | TGTCATCGTAAGGCAGGCCAT |
| 1786 | JAK2_002 | CAGAAATATCACCATTCTGAT |
| 1787 | JAK2_003 | CTTCATAGAATTGGCATTTCC |
| 1788 | JAK2_004 | TGGAAATGCCAATTCTATGAA |
| 1789 | JAK2_005 | CCAAGGGAATGGTAAAGATAC |
| 1790 | JAK2_006 | CCATTCCCTTGGGAAATCTGA |
| 1791 | JAK2_007 | TTCTGCAACATACTCCCCAGA |
| 1792 | JAK2_008 | CATCTGGGGAGTATGTTGCAG |
| 1793 | JAK2_009 | GAAGCAGCAATACAGATTTCT |
| 1794 | JAK2_010 | ATACTTACCACAAGCTTTAGA |
| 1795 | JAK2_011 | TCTGCTTCTTTTCTAGGTATC |
| 1796 | JAK2_012 | TAGGTATCACACCTGTGTATC |
| 1797 | JAK2_013 | ACTCATTAAAGCAAACATATT |
| 1798 | JAK2_014 | TGTTTCACTCATTAAAGCAAA |
| 1799 | JAK2_015 | CTTTAATGAGTGAAACAGAAA |
| 1800 | JAK2_016 | ATGAGTGAAACAGAAAGGATC |
| 1801 | JAK2_017 | CTGAAGAAAGTACCTTATTCT |
| 1802 | JAK2_018 | TTATCTTGTAGATTTTACTTT |
| 1803 | JAK2_019 | CTTTCCTCGTTGGTATTGCAG |
| 1804 | JAK2_020 | CTCGTTGGTATTGCAGTGGCA |
| 1805 | JAK2_021 | TCATGTCTTACCTCTTTGCTC |
| 1806 | JAK2_022 | CTTCAAATTTTTGGTTTTAGT |
| 1807 | JAK2_023 | TCCATCCGTGCACAAAATCAT |
| 1808 | JAK2_024 | GTTTTAGTGGCGGCATGATTT |
| 1809 | JAK2_025 | GTGGCGGCATGATTTTGTGCA |
| 1810 | JAK2_026 | ATGAGTCACAGGTACTTTTAT |
| 1811 | JAK2_027 | TGCACGGATGGATAAAAGTAC |
| 1812 | JAK2_028 | GCTATTCTCATCATATCTAAC |
| 1813 | JAK2_029 | TTTGGCTATTCTCATCATATC |
| 1814 | JAK2_030 | ATCGTTTTCTTTGGCTATTCT |
| 1815 | JAK2_031 | CAAAAGAAAATTACCTGATAG |
| 1816 | JAK2_032 | GTAAGAATGTCTTGTAGCTAG |
| 1817 | JAK2_033 | TCCCTAGCTACAAGACATTCT |
| 1818 | JAK2_034 | CTCGAATACATTTTGGTAAGA |
| 1819 | JAK2_035 | ACAAGGAAGCGAATAAGGTAC |
| 1820 | JAK2_036 | CATTGGCTGAATTGCTGAATA |
| 1821 | JAK2_037 | GCAGATTTATTCAGCAATTCA |
| 1822 | JAK2_038 | TGGCAGTGGCTTTGCATTGGC |
| 1823 | JAK2_039 | TTCAGCAATTCAGCCAATGCA |
| 1824 | JAK2_040 | AAGTTTCTGGCAGTGGCTTTG |
| 1825 | JAK2_041 | TAAGATACTTAAGTTTCAAGT |
| 1826 | JAK2_042 | CAGATTTATAAGATACTTAAG |
| 1827 | JAK2_043 | TCTGTGTAGAAGGCAGACTGC |
| 1828 | JAK2_044 | CTTCAAATTTCTCTGTGTAGA |
| 1829 | JAK2_045 | AAGTAAAAGAACCTGGAAGTG |
| 1830 | JAK2_046 | CAGTTATTATAATGGTTGCAA |
| 1831 | JAK2_047 | CAACCATTATAATAACTGGAA |
| 1832 | JAK2_048 | CCTCTTGACCACTGAATTCCA |
| 1833 | JAK2_049 | TGTTTCCCTCTTGACCACTGA |
| 1834 | JAK2_050 | TTTATGTTTCCCTCTTGACCA |
| 1835 | JAK2_051 | CATGCTTTTAATTATAGGATT |
| 1836 | JAK2_052 | ATTATAGGATTTACAGTTATA |
| 1837 | JAK2_053 | CAGTTATATTGCGATTTTCCT |
| 1838 | JAK2_054 | CTTGCTTAATACTGACATCAA |
| 1839 | JAK2_055 | CTAATATTATTGATGTCAGTA |
| 1840 | JAK2_056 | AACCCTCTTGGTTTGCTTGCT |
| 1841 | JAK2_057 | ATTTGAACCCTCTTGGTTTGC |
| 1842 | JAK2_058 | CCATCTTGCTTATGGATAGTT |
| 1843 | JAK2_059 | TTTTTCTTTTCTCTGCTTAGG |
| 1844 | JAK2_060 | TTTTCTCTGCTTAGGAAATTG |
| 1845 | JAK2_061 | TCTGCTTAGGAAATTGAACTT |
| 1846 | JAK2_062 | TCTTTCGTGTCATTAATTGAT |
| 1847 | JAK2_063 | GTGTCATTAATTGATGGATAT |
| 1848 | JAK2_064 | CAGAGGTAATGATGTGCATCT |
| 1849 | JAK2_065 | AAGCACGGCTGGAGGTGCTAC |
| 1850 | JAK2_066 | TATATTTTCAAGCACGGCTGG |
| 1851 | JAK2_067 | AGTCTGTATTACTCACGAAAT |
| 1852 | JAK2_068 | CTAATGGCAAAATCCATCCTA |
| 1853 | JAK2_069 | TTCAGTTTACTAATGGCAAAA |
| 1854 | JAK2_070 | CCTTTAGGATGGATTTTGCCA |
| 1855 | JAK2_071 | GGATGGATTTTGCCATTAGTA |
| 1856 | JAK2_072 | CCATTAGTAAACTGAAGAAAG |
| 1857 | JAK2_073 | TTAAAGTCCTTAGGACTGCAT |
| 1858 | JAK2_074 | ATAAATATTTTTTGACTTTTG |
| 1859 | JAK2_075 | TATTCAATGACATTTTCTCGC |
| 1860 | JAK2_076 | TAATTAAACTTATACAGCGAG |
| 1861 | JAK2_077 | TAATCAAACAGTGTTTATATT |
| 1862 | JAK2_078 | ATTACAAAAAATGAGAATGAA |
| 1863 | JAK2_079 | TCCCACTGAGGTTGTACTCTT |
| 1864 | JAK2_080 | AGACTGCTGAAGTTCTTCTTT |
| 1865 | JAK2_081 | CATCTGGTAACAATTCAAAAG |
| 1866 | JAK2_082 | AATTGTTACCAGATGGAAACT |
| 1867 | JAK2_083 | GTAAACTGGAAAATTATATTG |
| 1868 | JAK2_084 | GGGGACAGCATTTAGTAAACT |
| 1869 | JAK2_085 | GCTTTGGGGGACAGCATTTAG |
| 1870 | JAK2_086 | CAGTTTACTAAATGCTGTCCC |
| 1871 | JAK2_087 | CTAAATGCTGTCCCCCAAAGC |
| 1872 | JAK2_088 | TTTTTTCAGATAAATCAAACC |
| 1873 | JAK2_089 | AGATAAATCAAACCTTCTAGT |
| 1874 | JAK2_090 | TGATGTACCAACCTCACCAAC |
| 1875 | JAK2_091 | GTTCATATGAGTAGGCCTCTG |
| 1876 | JAK2_092 | TGAAACACCATTTGGTTCATA |
| 1877 | JAK2_093 | TGATTTTGTGAAACACCATTT |
| 1878 | JAK2_094 | ACAAAATCAGAAATGAAGATT |
| 1879 | JAK2_095 | TTTTACCTTTTTCTCTTGAAG |
| 1880 | JAK2_096 | CCTTTTTCTCTTGAAGAATGA |
| 1881 | JAK2_097 | TAAAAGTGCCTTGGCCAAGGC |
| 1882 | JAK2_098 | TCTTGAAGAATGAAAGCCTTG |
| 1883 | JAK2_099 | AAAATCTTTGTAAAAGTGCCT |
| 1884 | JAK2_100 | CAAAGATTTTTAAAGGCGTAC |
| 1885 | JAK2_101 | AAGGCGTACGAAGAGAAGTAG |
| 1886 | JAK2_102 | ATGCAGTTGACCGTAGTCTCC |
| 1887 | JAK2_103 | AAAGAACTTCTGTTTCATGCA |
| 1888 | JAK2_104 | TCCAGAACTTTTAAAAGAACT |
| 1889 | JAK2_105 | TGTGTGCTTTATCCAGAACTT |
| 1890 | JAK2_106 | AAAGTTCTGGATAAAGCACAC |
| 1891 | JAK2_107 | TACttttttttttCCTTAGTC |
| 1892 | JAK2_108 | CTTAGTCTTTCTTTGAAGCAG |
| 1893 | JAK2_109 | TTTGAAGCAGCAAGTATGATG |
| 1894 | JAK2_110 | AAGCAGCAAGTATGATGAGCA |
| 1895 | JAK2_111 | AAACCAAATGCTTGTGAGAAA |
| 1896 | JAK2_112 | TCACAAGCATTTGGTTTTAAA |
| 1897 | JAK2_113 | GTTTTAAATTATGGAGTATGT |
| 1898 | JAK2_114 | CTTACTCTCGTCTCCACAGAC |
| 1899 | JAK2_115 | AATTATGGAGTATGTGTCTGT |
| 1900 | JAK2_116 | CAAACTCCTGAACCAGAATAT |
| 1901 | JAK2_117 | ATGCAGATATTCTGGTTCAGG |
| 1902 | JAK2_118 | AGATATGTATCTAGTGATCCA |
| 1903 | JAK2_119 | TTCTTTTTCAGATATGTATCT |
| 1904 | JAK2_120 | TAAAATTTGGATCACTAGATA |
| 1905 | JAK2_121 | GATCACTAGATACATATCTGA |
| 1906 | JAK2_122 | TACAATTTTTATTCTTTTTCA |
| 1907 | JAK2_123 | CATAATATATTTATACAATTT |
| 1908 | JAK2_124 | GCAACTTCAAGTTTCCATAAT |
| 1909 | JAK2_125 | ACTCTAATAGGAAGAAAACAC |
| 1910 | JAK2_126 | GCACATACATTCCCATGAATA |
| 1911 | JAK2_127 | CTGTCTTCCTGTCTTCTTCTC |
| 1912 | JAK2_128 | ATGAAAGGAGGATTTCCTGTC |
| 1913 | JAK2_129 | ATCAAACTTAGTGATCCTGGC |
| 1914 | JAK2_130 | GCAAAACTGTAATACTAATGC |
| 1915 | JAK2_131 | AAAGTTCTTCAGGAGAGAATA |
| 1916 | JAK2_132 | AATGCATTCAGGTGGTACCCA |
| 1917 | JAK2_133 | GGATTTTCAATGCATTCAGGT |
| 1918 | JAK2_134 | AATTTTTAGGATTTTCAATGC |
| 1919 | JAK2_135 | TCTGTTGCCAAATTTAAATTT |
| 1920 | JAK2_136 | AATTTGGCAACAGACAAATGG |
| 1921 | JAK2_137 | CCACAAAGTGGTACCAAAACT |
| 1922 | JAK2_138 | GCAACAGACAAATGGAGTTTT |
| 1923 | JAK2_139 | TCTCCTCCACTGCAGATTTCC |
| 1924 | JAK2_140 | GTACCACTTTGTGGGAAATCT |
| 1925 | JAK2_141 | TGGGAAATCTGCAGTGGAGGA |
| 1926 | JAK2_142 | AGAATCCAGAGCACTTAGAGG |
| 1927 | JAK2_143 | TGGTTCTTTAATTATAGAAGC |
| 1928 | JAK2_144 | ATTATAGAAGCTACAATTTTA |
| 1929 | JAK2_145 | GTGCAGGAAGCTGATGCCTAT |
| 1930 | JAK2_146 | TGAAGATAGGCATCAGCTTCC |
| 1931 | JAK2_147 | CTAATTCTGCCCACTTTGGTG |
| 1932 | JAK2_148 | TAAGGTTTGCTAATTCTGCCC |
| 1933 | JAK2_149 | AGGCCTTCTTTCAGAGCCATC |
| 1934 | JAK2_150 | AGAGCCATCATACGAGATCTT |
| 1935 | JAK2_151 | TGTTAATAGTTCATAATCTGG |
| 1936 | JAK2_152 | TTTCTCCAGATTATGAACTAT |
| 1937 | JAK2_153 | TCCAGATTATGAACTATTAAC |
| 1938 | JAK2_154 | GTAACATGTCATTTTCTGTTA |
| 1939 | JAK2_155 | TGGTGCCTTTGAAGACCGGGA |
| 1940 | JAK2_156 | AAATGTCTCTCTTCAAACTGT |
| 1941 | JAK2_157 | AAGACCGGGATCCTACACAGT |
| 1942 | JAK2_158 | AAGAGAGACATTTGAAATTTC |
| 1943 | JAK2_159 | CCTTGCCAAGTTGCTGTAGAA |
| 1944 | JAK2_160 | AAATTTCTACAGCAACTTGGC |
| 1945 | JAK2_161 | AAAAAATTCTGACAATTTACC |
| 1946 | JAK2_162 | TACAGCAACTTGGCAAGGTAA |
| 1947 | JAK2_163 | GGGTAATTTTGGGAGTGTGGA |
| 1948 | JAK2_164 | GGAGTGTGGAGATGTGCCGGT |
| 1949 | JAK2_165 | CAGCGACCACCTCCCCAGTGT |
| 1950 | JAK2_166 | AAAGTCTCTTAGGTGCTCTTC |
| 1951 | JAK2_167 | CCTTTCAAAGTCTCTTAGGTG |
| 1952 | JAK2_168 | AATTTCCCTTTCAAAGTCTCT |
| 1953 | JAK2_169 | AGGATTTCAATTTCCCTTTCA |
| 1954 | JAK2_170 | AAAGGGAAATTGAAATCCTGA |
| 1955 | JAK2_171 | CAATGTTGTCATGCTGTAGGG |
| 1956 | JAK2_172 | AATGGGCAGCTTACCAGCACT |
| 1957 | JAK2_173 | CACCTTTATGTTAAAAGGTCG |
| 1958 | JAK2_174 | TGTTAAAAGGTCGGCGTAATC |
| 1959 | JAK2_175 | AAGATAGTCTCGTAAACTTCC |
| 1960 | JAK2_176 | TGTTTTTGAAGATAGTCTCGT |
| 1961 | JAK2_177 | CCATATGGAAGTTTACGAGAC |
| 1962 | JAK2_178 | CGAGACTATCTTCAAAAACAT |
| 1963 | JAK2_179 | TGTGATCTATCCGTTCTTTAT |
| 1964 | JAK2_180 | TACCAAGATACTCCATACCCT |
| 1965 | JAK2_181 | TCCATAGGGTATGGAGTATCT |
| 1966 | JAK2_182 | TCGTTGCCAGATCCCTGTGGA |
| 1967 | JAK2_183 | ACTCTGTTCTCGTTCTCCACC |
| 1968 | JAK2_184 | GTTAACCCAAAATCTCCAATT |
| 1969 | JAK2_185 | TCTTGTGGCAAGACTTTGGTT |
| 1970 | JAK2_186 | TAGTATTCTTTGTCTTGTGGC |
| 1971 | JAK2_187 | GGTTAACCAAAGTCTTGCCAC |
| 1972 | JAK2_188 | CTTTATAGTATTCTTTGTCTT |
| 1973 | JAK2_189 | ACCAGGTTCTTTTACTTTATA |
| 1974 | JAK2_190 | TCATACTGAAATATACTCACC |
| 1975 | JAK2_191 | CAGGTATGCTCCAGAATCACT |
| 1976 | JAK2_192 | TGTGGCCTCAGATGTTTGGAG |
| 1977 | JAK2_193 | GAGCTTTGGAGTGGTTCTGTA |
| 1978 | JAK2_194 | GAGTGGTTCTGTATGAACTTT |
| 1979 | JAK2_195 | CTCTTCTCAATGTATGTGAAA |
| 1980 | JAK2_196 | ACATACATTGAGAAGAGTAAA |
| 1981 | JAK2_197 | TCATTGCCAATCATACGCATA |
| 1982 | JAK2_198 | TTTTAGGAATTTATGCGTATG |
| 1983 | JAK2_199 | GGAATTTATGCGTATGATTGG |
| 1984 | JAK2_200 | TGCGTATGATTGGCAATGACA |
| 1985 | JAK2_201 | ATAGAACTTTTGAAGAATAAT |
| 1986 | JAK2_202 | AAGAATAATGGAAGATTACCA |
| 1987 | JAK2_203 | GTTTATTTTCTCCTTTACAGA |
| 1988 | JAK2_204 | TTTTCTCCTTTACAGATCTAT |
| 1989 | JAK2_205 | TCCTTTACAGATCTATATGAT |
| 1990 | JAK2_206 | CAGATCTATATGATCATGACA |
| 1991 | JAK2_207 | CATTATTGTTCCAGCATTCTG |
| 1992 | JAK2_208 | ATCCACTCGAAGAGCTAGATC |
| 1993 | JAK2_209 | GGGATCTAGCTCTTCGAGTGG |
| 1994 | JAK2_210 | ATCCAGCCATGTTATCCCTTA |
| 1995 | JAK2_211 | TTTCATCCAGCCATGTTATCC |
| 1996 | TRAC043 | GAGTCTCTCAGCTGGTACACG |
| 1997 | TRAC049 | TCTGTGATATACACATCAGAA |
| 1998 | TRAC051 | TTGCTCCAGGCCACAGCACTG |
| 1999 | TRBC1_2_001 | GGTGTGGGAGATCTCTGCTTC |
| 2000 | TRBC1_2_003 | AGCCATCAGAAGCAGAGATCT |
| 2001 | CD3E_24 | AGATCCAGGATACTGAGGGCA |
| 2002 | CD3E_34 | CTTCCTCTGGGGTAGCAGACA |
| 2003 | CD3E_40 | CCCTCCTTCCTCCGCAGGACA |
| 2004 | CD3D_002 | CCCTTTAGTGAGCCCCTTCAA |
| 2005 | CD3D_003 | GTGAGCCCCTTCAAGATACCT |
| 2006 | CD3D_005 | CCAGGTCCAGTCTTGTAATGT |
| 2007 | CD3G_001 | CCGGAGGACAGAGACTGACAT |
| 2008 | CD3G_023 | CAGGTACTTTGGCCCAGTCAA |
| 2009 | CD247_001 | TGAGGGAAAGGACAAGATGAA |
| 2010 | CD247_002 | ACCGCGGCCATCCTGCAGGCA |
| 2011 | CD247_004 | GGATCCAGCAGGCCAAAGCTC |
| 2012 | B2M_30 | AGTGGGGGTGAATTCAGTGTA |
| 2013 | B2M_4 | CTCACGTCATCCAGCAGAGAA |
| 2014 | NLRC5_002 | GGGAAGGCTGGCATGGGCAAG |
| 2015 | NLRC5_011 | GGGCCACTCACAGCCTGCTGA |
| 2016 | NLRC5_019 | ATGGCTGTCCCCTGGAGCCCC |
| 2017 | CIITA_65 | GCAGCACGTGGTACAGGAGCT |
| 2018 | CIITA_80 | CAAGGACTTCAGCTGGGGGAA |
| 2019 | CIITA_36 | TGGGCTCAGGTGCTTCCTCAC |

### B. Methods for Reducing Immunogenicity of Cells

In certain embodiments, provided herein are methods. In certain embodiments, provided herein are methods for engineering cells, such as human cells. In certain embodiments, provided herein are methods for engineering cells to reduce the immunogenicity of the engineered cells. In certain embodiments, provided herein are methods for engineering cells to be introduced into a recipient that is allogeneic to the individual that was the source of the cells (also referred to herein as “allogeneic cells”) that reduce the immunogenicity of the engineered, allogeneic cells.

In certain embodiments, provided herein are methods for generating one or more modifications in the genome of a target cell. In certain embodiments, the method can generate at least 1, 2, 3, 4, 5, 6, 7, 8, 9, 10, 15, 20, 25, 30, 35, 40, 45, or 50 and/or no more than 2, 3, 4, 5, 6, 7, 8, 9, 10, 15, 20, 25, 30, 35, 40, 45, 50, or 100 genomic modifications, for example, 1-100 genomic modifications, preferably 1-20 genomic modifications, either simultaneously or sequentially (see Multiplexing section below). In certain embodiments, a first genomic modification is introduced into one or more target cells, wherein the target cell comprises a wild-type cell or a cell comprising one or more genomic modifications (see Cells comprising genomic modifications section above). In certain embodiments, the target cell comprises one or more of the modified cells as described in the Cells comprising genomic modifications section (above). In certain embodiments, the method comprises generating one or more genomic modifications in one or more target cells, wherein the one or more genomic modifications are generated simultaneously, e.g., in a single cell by introduction of all necessary components to produce the desired genomic modifications. In certain embodiments, the method comprises generating one or more genomic modifications in one or more target cells, wherein one or more of the genomic modifications are generated sequentially, e.g., where a portion of desired genetic modifications are produced in a parent cell and the remaining desired genetic modifications are produced in one or more generations of progeny from the parent cell. In certain embodiments wherein one or more genomic modifications are introduced sequentially, the one or more genomic modifications may be introduced in any suitable quantity, order, and/or combination. For example, when introducing three genomic modifications (A, B, and C) into one or more cells, the three genomic modifications can be introduced in any one of the following orders: (1) A then B then C; (2) A then C then B; (3) A and B then C; (4) A then B and C; (5) A and C then B; (6) A then C and B; (7) B then A then C; (8) B then C then A; (9) B and A then C; (10) B then A and C; (11) B and C then A; (12) B then C and A; (13) C then A then B; (14) C then B then A; (15) C and A then B; (16) C then A and B; (17) C then B and A; (18) C and B then A; or (19) A and B and C.

In certain embodiments, provided herein are methods for engineering one or more human cells. Any suitable human cell or cells may be used. In certain embodiments, the cells comprise one or more human stem cells or human immune cells. In certain embodiments, the cells comprise one or more human cells comprising an immune cell comprising a neutrophil, cosinophil, basophil, mast cell, monocyte, macrophage, dendritic cell, natural killer cell, a lymphocyte, or a combination thereof. In certain embodiments, the cells comprise one or more T cells. In certain embodiments, the cells comprise one or more chimeric antigen receptor (CAR)-T cells. In certain embodiments, the CAR T cell comprises a CAR polypeptide or portion thereof. In certain embodiments, the CAR T cell comprises two or more CAR polypeptides or portions thereof. In certain embodiments, the CAR T cell comprises a dual CAR, wherein the dual CAR comprises a first CAR polypeptide or portions thereof, and a second CAR polypeptide or portion thereof, wherein the second CAR polypeptide is different than the first CAR polypeptide and the first and second CAR polypeptides are separate. In certain embodiments, the first and second CAR polypeptides are linked by a polypeptide linker. In certain embodiments, the cells comprise one or more human stem cells comprising a human pluripotent, multipotent stem cell, embryonic stem cell, induced pluripotent stem cell, hematopoietic stem cell, a CD34+ cell, a combination thereof. In preferred embodiments, the cells comprise one or more hematopoietic stem cells. In more preferred embodiments, the cells comprise one or more CD34+ stem cells. In even more preferred embodiments, the cells comprise one or more induced pluripotent stem cells (iPSC). In certain embodiments, the cells comprise an allogeneic cell.

In certain embodiments, the one or more cells comprising one or more introduced genomic modifications are either grown, e.g., expanded, or differentiated, for example an iPSC differentiated into a T cell. In certain embodiments wherein two or more genomic modifications are introduced sequentially, the one or more target cells are expanded after introduction of the first set of genomic modifications, wherein the second set of genomic modifications are introduced into the progeny of the first set of cells. In certain embodiments, the stem cells are differentiated before or after introduction of one or more genomic modifications. In certain embodiments, the stem cells are differentiated after introduction of one or more genomic modifications.

In certain embodiments, one or more genomic modifications are introduced into a population of cells, wherein the resulting cell population comprises a plurality of cell populations each having received a different set of genomic modifications (see Cell populations section above). For example, when introducing three genomic modifications (A, B, C) into a population of cells, either sequentially and/or simultaneously, the resulting plurality of cell populations could potentially compromise any number and/or combination of the following cell populations: (1) A, (2) AB, (3) AC, (4) ABC, (5) B, (6) BC, (7) C, and/or (8) no genomic modifications. In certain embodiments, each cell population in the plurality of cell populations can be present at any percentage relative to the other cell populations, wherein the relative percentage of each population is affected by a number of factors including but not limited to delivery efficiency of the editing components, quality of the editing components, concentration of the editing components, relative efficiency and specificity of the editing events, vitality of the cells, and/or viability of the cells before or after introduction of the one or more genomic modifications.

In certain embodiments, provided herein are methods for engineering cells comprising delivering one or more site-specific nucleases to the one or more target cells. In certain embodiments, the one or more site-specific nucleases are delivered to the target cells as a polypeptide. In certain embodiments, the one or more site-specific nucleases are combined with a compatible guide nucleic acid to comprise a nucleic acid-guided nuclease system, e.g., a CRISPR/cas system. In certain embodiments, one or more polynucleotides encoding for one or more components of the nuclease system are delivered to the target cells. In a preferred embodiment, the nucleic acid-guided nuclease system comprises a Type V nuclease, more preferably a Type V-A nuclease, even more preferably a MAD2, MAD7, ART2, ART11, ART11* nucleases, yet more preferably a MAD7 nuclease.

In certain embodiments, one more guide nucleic acids comprising a spacer sequence at least partially complementary a target nucleotide sequence within a site wherein one or more genomic modifications are to be introduced are delivered to the target cells. In certain embodiments, one or more nucleic acid-guided nucleases are delivered to the target cells. In certain embodiments, a combination of one or more guide nucleic acids and nucleic acid-guided nucleases are delivered to the target cells, wherein the one or more nucleic acid-guided nucleases are optionally complexed with a guide nucleic acid (e.g., see Ribonucleoprotein (RNP) section below). In certain embodiments, one or more fully formed nucleic acid-guided nuclease complexes are delivered, e.g., RNP. In certain cases, any one of the embodiments as described in the Guide nucleic acids and donor templates section can be delivered to the target cell.

In certain embodiments, provided herein is a method of producing a non-immunogenic cell. In certain embodiments, provided herein in a method of producing a non-immunogenic stem cell or immune cell. In certain embodiments, provided herein is a method of producing a non-immunogenic CAR T cell. In certain embodiments provided herein is a method of producing a non-immunogenic CAR T cell comprising (1) modifying a genome of a cell to reduce or eliminate cell surface expression of active HLA-1 proteins in the cell and its progeny, (2) introducing intro the genome of the cell or one or more of its progeny a first polynucleotide coding for surface expression of a first CAR or portion thereof specific for a first antigen, and (3) introducing into the genome of the cell or one or more of its progeny a second polynucleotide coding for surface expression of a second CAR or portion thereof specific for a second antigen. In certain embodiments, the method further comprises modifying a genome of a cell to reduce or eliminate surface expression of active HLA-1 proteins comprising introducing a genomic modification into a B2M gene that partially or completely inactivates the B2M gene. In certain embodiments, the B2M gene is completely inactivated. In certain embodiments wherein the B2M gene is partially or complete inactivated, a first transgene coding for a B2M-HLA-1 subunit fusion protein is introduced. In certain embodiments, the B2M-HLA-1 subunit fusion protein comprising a HLA-1 subunit comprising HLA-C, -E, or -G. In a preferred embodiment, the HLA-1 subunit comprises HLA-E or -G. In certain embodiments, the first and/or second CAR or portion thereof comprises any one of the CARs as described in the Surface proteins & CARs section above. In certain embodiments, the method further comprises modifying the genome of the cell or one of its progeny to reduce or eliminate surface expression of one or more subunits of an HLA-2 protein. In certain embodiments, the one or more subunits of an HLA-2 protein is modified by introducing a genomic modification into a gene coding for a transcription factor for one or more gene encoding the one or more subunits of an HLA-2 protein. In certain embodiments, the genomic modification in the transcription factor regulating expression of one or more subunits of an HLA-2 protein at least partially or completely inactivates the transcription factor. In certain embodiments, the transcription factor is completely inactivated. In a preferred embodiment, the transcription factor comprises CIITA. In certain embodiments, the method further comprises delivering into the cell a nucleic acid-guided nuclease system, or one or more polynucleotides encoding for one or more parts of the system, comprising a nucleic acid-guided nuclease and a guide nucleic acid compatible with and capable of binding to and activating the nucleic acid-guided nuclease, wherein the guide nucleic acid comprises a targeter nucleic acid comprising a targeter stem sequence and a spacer sequence, wherein the spacer sequence is complementary to a target nucleotide sequence within a target polynucleotide of a genome of a human target cell and a modulator nucleic acid comprising a modulator stem sequence complementary to the targeter stem sequence, and, optionally, a 5′ sequence, wherein the nucleic acid-guided nuclease system target and cleave at least one strand in the target polynucleotide at or near the target nucleotide sequence. In certain embodiments, the nuclease comprises any suitable nuclease. In certain embodiments, the nuclease comprises any suitable nuclease as described in the Cas proteins section (below). In certain embodiments, the nuclease comprises a Type V nuclease, preferably a Type V-A nuclease, an ART2, ART11, ART11*, MAD2, and/or MAD7 nuclease, even more preferably a MAD7 nuclease. In certain embodiments, the nucleic acid guided nuclease system comprises a guide nucleic acid comprising a single polynucleotide and/or a guide nucleic acid comprising one or more polynucleotides, e.g., a dual guide nucleic acid, preferably the guide nucleic acid comprises a dual guide nucleic acid capable of binding to and activating a nucleic acid-guided nuclease, that, in a naturally occurring system, is activated by a single crRNA in the absence of a tracrRNA. In certain embodiments, the guide nucleic acid comprises one or more chemical modifications as described in the gNA modifications section (below). In certain embodiments, the method further comprises delivering one or more donor templates as described in the Donor templates section below. In certain embodiments, at least a portion of the donor template is inserted through an innate cell repair mechanism initiated by the generated of one or more strand breaks at or near a target nucleotide sequence by the one or more nucleic acid-guided nucleases. In certain embodiments, delivery of the one or more components for genome engineering is by electroporation.

In certain embodiments, provided herein is a method for producing a population of non-immunogenic CAR T cells comprising (1) modifying a genome of a first cell to reduce or eliminate cell surface expression of HLA-1 proteins in the first cell and its progeny, (2) introducing into the genome of the first cell a first polynucleotide coding for surface expression of a first CAR specific for a first antigen on the first cell, (3) modifying a genome of a second cell to reduce or eliminate cell surface expression of HLA-1 proteins in the second cell and its progeny, and (4) introducing into the genome of the second cell a second polynucleotide coding for surface expression of a second CAR specific for a second antigen on the second cell, wherein the first and second cells are the same cell, the first cell is a progeny of the second cell, or the second cell is a progeny of the first cell. Steps (1) through (4) may be performed in any suitable order.

In certain embodiments, provided herein is a method for producing a population of non-immunogenic CAR T cells comprising (1) modifying a genome of a first cell to reduce or eliminate cell surface expression of HLA-1 proteins in the first cell and its progeny, (2) introducing into the genome of the first cell a first polynucleotide coding for surface expression of a first CAR specific for a first antigen on the first cell, (3) modifying a genome of a second cell to reduce or eliminate cell surface expression of HLA-1 proteins in the second cell and its progeny, and (4) introducing into the genome of the second cell a second polynucleotide coding for surface expression of a second CAR specific for a second antigen on the second cell, wherein the first and second cells are the same cell, the first cell is a progeny of the second cell, or the second cell is a progeny of the first cell. In certain embodiments, steps (1) through (4) are performed simultaneously, wherein the first, second, third, and fourth cells are the same cell. In certain embodiments, one or more of steps (1) through (4) are performed sequentially, for example any one of the following sequential permutations may be employed: ABCD, ABDC, ACBD, ACDB, ADBC, ADCB, BACD, BADC, BCAD, BCDA, BDAC, BDCA, CABD, CADB, CBAD, CBDA, CDAB, CDBA, DABC, DACB, DBAC, DBCA, DCAB, DCBA. In certain embodiments, one or more of the steps may be performed simultaneously wherein at least one step is performed sequentially, for example A then BCD or A and B then C and D.

In certain embodiments, provided herein is a method of modifying a genome of a human cell comprising (1) modifying a B2M gene in the genome to reduce or eliminate expression of the B2M gene, (2) modifying a T cell receptor (TCR) subunit gene in the genome to reduce or eliminate expression of the subunit, and (3) modifying a CIITA gene in the genome to reduce or eliminate expression of the CIITA gene, wherein at least 2 of (a) to (c) are performed sequentially, not simultaneously, thereby producing a modified human cell.

### II. ENGINEERED NON-NATURALLY OCCURRING DUAL GUIDE CRISPR-CAS SYSTEMS

A CRISPR-Cas system generally comprises a Cas protein and one or more guide nucleic acids (gNAs). The Cas protein can be directed to a specific location in a double-stranded DNA target by recognizing a protospacer adjacent motif (PAM) in the non-target strand of the DNA, and the one or more guide nucleic acids can be directed to a specific location by hybridizing with a target nucleotide sequence, also referred to herein as a target sequence, in the target strand of the target polynucleotide. Typically, both PAM recognition and target nucleotide sequence hybridization are required for stable binding of a CRISPR-Cas complex to the DNA target and, if the Cas protein has an effector function (e.g., nuclease activity), activation of the effector function. As a result, when creating a CRISPR-Cas system, a guide nucleic acid can be designed to comprise a nucleotide sequence called a spacer sequence that is at least partially complementary to and can hybridize with a target nucleotide sequence, where target nucleotide sequence is located adjacent to a PAM in an orientation operable with the Cas protein. It has been observed that not all CRISPR-Cas systems designed by these criteria are equally effective. The larger polynucleotide in which a target nucleotide sequence is located may be referred to as a target polynucleotide; e.g., a chromosome or other genomic DNA, or portion thereof, or any other suitable polynucleotide within which a target nucleotide sequence is located. The target polynucleotide in double stranded DNA comprises two strands. The strand of the DNA duplex to which the spacer sequence is complementary herein is called the “target strand,” while the strand to which the spacer sequence shares sequence identity herein is called the “non-target strand.”

Two distinct classes of CRISPR-Cas systems have been identified. Class 1 CRISPR-Cas systems utilize multi-protein effector complexes, whereas class 2 CRISPR-Cas systems utilize single-protein effectors (see, Makarova et al. (2017) CELL, 168:328). Among the types of class 2 CRISPR-Cas systems, type II and type V systems typically target DNA and type VI systems typically target RNA (id.). Naturally occurring type II effector complexes include Cas9, CRISPR RNA (crRNA), and trans-activating CRISPR RNA (tracrRNA), but the crRNA and tracrRNA can be fused as a single guide RNA in an engineered system for simplicity (see, Wang et al. (2016) ANNU. REV. BIOCHEM., 85:227). Certain naturally occurring type V systems, such as type V-A, type V-C, and type V-D systems, do not require tracrRNA and use crRNA alone as the guide for cleavage of target DNA (see, Zetsche et al. (2015) CELL, 163:759; Makarova et al. (2017) CELL, 168:328.

Naturally occurring type II CRISPR-Cas systems (e.g., CRISPR-Cas9 systems) generally comprise two guide nucleic acids, called crRNA and tracrRNA, which form a complex by nucleotide hybridization. Single guide nucleic acids capable of activating type II Cas nucleases have been developed, for example, by linking the crRNA and the tracrRNA (see, e.g., U.S. Pat. Nos. 10,266,850 and 8,906,616). Naturally occurring type II Cas proteins comprise a RuvC-like nuclease domain and an HNH endonuclease domain, and recognize a 3′ G-rich PAM located immediately downstream from the target nucleotide sequence, the orientation determined using the non-target strand (i.e., the strand not hybridized with the spacer sequence) as the coordinate. The CRISPR-Cas systems cleave a double-stranded DNA to generate a blunt end. The cleavage site is generally 3-4 nucleotides upstream from the PAM on the non-target strand.

Naturally occurring Type V-A, Type V-C, and Type V-D CRISPR-Cas systems lack a tracrRNA and rely on a single crRNA to guide the CRISPR-Cas complex to the target polynucleotide. Dual guide nucleic acids capable of activating type V-A, type V-C, or type V-D Cas nucleases have been developed, for example, by splitting the single crRNA into a targeter nucleic acid and a modulator nucleic acid (see, e.g., International (PCT) Application Publication No. WO 2021/067788). Naturally occurring type V-A Cas proteins comprise a RuvC-like nuclease domain but lack an HNH endonuclease domain, and recognize a 5′ T-rich PAM located immediately upstream from the target nucleotide sequence, the orientation determined using the non-target strand (i.e., the strand not hybridized with the spacer sequence) as the coordinate. These CRISPR-Cas systems cleave a double-stranded DNA to generate a staggered double-stranded break rather than a blunt end. The cleavage site is distant from the PAM site (e.g., separated by at least 10, 11, 12, 13, 14, or 15 nucleotides downstream from the PAM on the non-target strand and/or separated by at least 15, 16, 17, 18, or 19 nucleotides upstream from the sequence complementary to PAM on the target strand).

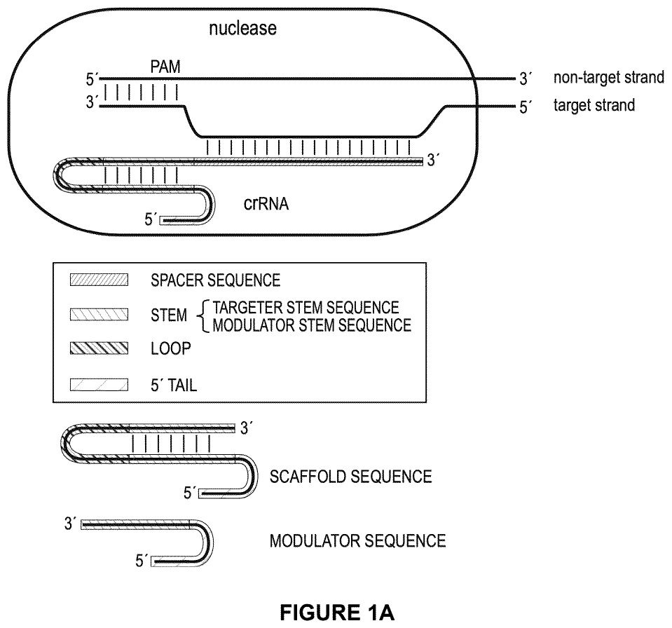

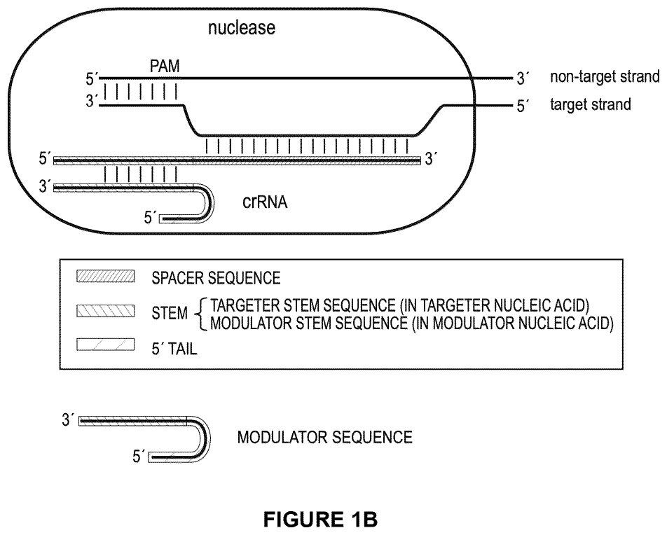

Elements in an exemplary single guide CRISPR Cas system, e.g., a type V-A CRISPR-Cas system, are shown in FIG. 1A. The single gNA can also be called a “crRNA” or “single gRNA” where it is present in the form of an RNA. It can comprise, from 5′ to 3′, an optional 5′ sequence, e.g., a tail, a modulator stem sequence, a loop, a targeter stem sequence complementary to the modulator stem sequence, and a spacer sequence that is at least partially complementary to and can hybridize with a target sequence in the target strand of the target polynucleotide. Where a 5′ tail is present, the sequence including the 5′ tail and the modulator stem sequence can also be called a “modulator sequence” herein. A fragment of the single guide nucleic acid from the optional 5′ tail to the targeter stem sequence, also called a “scaffold sequence” herein, bind the Cas protein. In addition, the PAM in the non-target strand of the target DNA binds the Cas protein.

Elements in an exemplary dual guide type CRISPR Cas system, e.g., a dual guide type V-A CRISPR-Cas system are shown in FIG. 1B. The first guide nucleic acid, which can be called a “modulator nucleic acid” herein, comprises, from 5′ to 3′, an optional 5′ tail and a modulator stem sequence. Where a 5′ tail is present, the sequence including the 5′ tail and the modulator stem sequence can also called a “modulator sequence” herein. The second guide nucleic acid, which can be called “targeter nucleic acid” herein, comprises, from 5′ to 3′, a targeter stem sequence complementary to the modulator stem sequence and a spacer sequence that is at least partially complementary to and can hybridize with the target sequence in the target strand of the target polynucleotide. The duplex between the modulator stem sequence and the targeter stem sequence, plus the optional 5′ tail, constitute a structure that binds the Cas protein. In addition, the PAM in the non-target strand of the target DNA binds the Cas protein. It is understood that, in a dual gNA, e.g., dual gRNA, the targeter nucleic acid and the modulator nucleic acid, while not in the same nucleic acids, i.e., not linked end-to-end through a traditional internucleotide bond, can be covalently conjugated to each other through one or more chemical modifications introduced into these nucleic acids, thereby increasing the stability of the double-stranded complex and/or improving other characteristics of the system.

The terms “targeter stem sequence” and “modulator stem sequence,” as used herein, can refer to a pair of nucleotide sequences in one or more guide nucleic acids that hybridize with each other. When a targeter stem sequence and a modulator stem sequence are contained in a single guide nucleic acid, the targeter stem sequence is proximal to a spacer sequence designed to hybridize with a target nucleotide sequence, and the modulator stem sequence is proximal to the targeter stem sequence. When a targeter stem sequence and a modulator stem sequence are in separate nucleic acids, the targeter stem sequence is in the same nucleic acid as a spacer sequence designed to hybridize with a target nucleotide sequence. In a CRISPR-Cas system that naturally includes separate crRNA and tracrRNA (e.g., a type II system), the duplex formed between the targeter stem sequence and the modulator stem sequence corresponds to the duplex formed between the crRNA and the tracrRNA. In a CRISPR-Cas system that naturally includes a single crRNA but no tracrRNA (e.g., a type V-A system), the duplex formed between the targeter stem sequence and the modulator stem sequence corresponds to the stem portion of a stem-loop structure in the scaffold sequence of the crRNA. It is understood that 100% complementarity is not required between the targeter stem sequence and the modulator stem sequence. In a type V-A CRISPR-Cas system, however, the targeter stem sequence is typically 100% complementary to the modulator stem sequence.

### A. Cas Proteins

A guide nucleic acid, either as a single guide nucleic acid alone (targeter and modulator nucleic acids are part of a single polynucleotide) or as a dual gNA comprising separate targeter nucleic acid used in combination with a cognate modulator nucleic acid, is capable of binding a CRISPR Associated (Cas) protein, e.g., a Cas nuclease. In certain embodiments, the guide nucleic acid, either as a single guide nucleic acid alone (targeter and modulator nucleic acids are part of a single polynucleotide) or as a dual gNA comprising separate targeter nucleic acid used in combination with a cognate modulator nucleic acid, is capable of activating a Cas nuclease. A gNA capable of activating a particular Cas nuclease is said to be “compatible” with the Cas nuclease; a Cas nuclease capable of being activated by a particular gNA is said to be “compatible” with the gNA.

The terms “CRISPR-Associated protein,” “Cas protein,” and “Cas,” as used interchangeably herein, can refer to a naturally occurring Cas protein or an engineered Cas protein. Non-limiting examples of Cas protein engineering include but are not limited to mutations and modifications of the Cas protein that alter the activity of the Cas, alter the PAM specificity, broaden the range of recognized PAMs, and/or reduce the ability to modify one or more off-target loci as compared to a corresponding unmodified Cas. In certain embodiments, the altered activity of engineered Cas comprises altered ability (e.g., specificity or kinetics) to bind a naturally occurring gNA, e.g., gRNA or engineered gNA, e.g., gRNA, altered ability (e.g., specificity or kinetics) to bind a target nucleotide sequence, altered processivity of nucleic acid scanning, and/or altered effector (e.g., nuclease) activity. A Cas protein having nuclease activity can be referred to as a “CRISPR-Associated nuclease” or “Cas nuclease,” or simply “nuclease,” as used interchangeably herein.

In certain embodiments, the Cas protein is a type V-A, type V-C, or type V-D Cas protein. In certain embodiments, the Cas protein is a type V-A Cas protein. In other embodiments, the Cas protein is a type II Cas protein, e.g., a Cas9 protein.

In certain embodiments, a type V-A Cas nucleases comprises Cpf1. Cpf1 proteins are known in the art and are described, e.g., in U.S. Pat. Nos. 9,790,490 and 10,113,179. Cpf1 orthologs can be found in various bacterial and archacal genomes. For example, in certain embodiments, the Cpf1 protein is derived from Francisella novicida U112 (Fn), Acidaminococcus sp. BV3L6 (As), Lachnospiraceae bacterium ND2006 (Lb), Lachnospiraceae bacterium MA2020 (Lb2), Candidatus Methanoplasma termitum (CMt), Moraxella bovoculi 237 (Mb), Porphyromonas crevioricanis (Pc), Prevotella disiens (Pd), Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Eubacterium eligens, Leptospira inadai, Porphyromonas macacae, Prevotella bryantii, Proteocatella sphenisci, Anaerovibrio sp. RM50, Moraxella caprae, Lachnospiraceae bacterium COE1, or Eubacterium coprostanoligenes.

In certain embodiments, a type V-A Cas nuclease comprises AsCpf1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 3 of International (PCT) Application Publication No. WO 2021/158918. In certain embodiments, a type V-A Cas protein comprises the amino acid sequence set forth in SEQ ID NO: 3 of International (PCT) Application Publication No. WO 2021/158918.

In certain embodiments, a type V-A Cas nuclease comprises LbCpf1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 4 of International (PCT) Application Publication No. WO 2021158918. In certain embodiments, a type V-A Cas protein comprises the amino acid sequence set forth in SEQ ID NO: 4 of International (PCT) Application Publication No. WO 2021/158918.

In certain embodiments, a type V-A Cas nuclease comprises FnCpf1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 5 of International (PCT) Application Publication No. WO 2021158918. In certain embodiments, a type V-A Cas protein comprises the amino acid sequence set forth in SEQ ID NO: 5 of International (PCT) Application Publication No. WO 2021/158918.

In certain embodiments, a type V-A Cas nuclease comprises Prevotella bryantii Cpf1 (PbCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 6 of International (PCT) Application Publication No. WO 2021/158918. In certain embodiments, a type V-A Cas protein comprises the amino acid sequence set forth in SEQ ID NO: 6 of International (PCT) Application Publication No. WO 2021/158918.

In certain embodiments, a type V-A Cas nuclease comprises Proteocatella sphenisci Cpf1 (PsCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 7 of International (PCT) Application Publication No. WO 2021158918. In certain embodiments, a type V-A Cas protein comprises the amino acid sequence set forth in SEQ ID NO: 7 of International (PCT) Application Publication No. WO 2021/158918.

In certain embodiments, a type V-A Cas nuclease comprises Anaerovibrio sp. RM50 Cpf1 (As2Cpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 8 of International (PCT) Application Publication No. WO 2021158918. In certain embodiments, a type V-A Cas protein comprises the amino acid sequence set forth in SEQ ID NO: 8 of International (PCT) Application Publication No. WO 2021/158918.

In certain embodiments, a type V-A Cas nuclease comprises Moraxella caprae Cpf1 (McCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 9 of International (PCT) Application Publication No. WO 2021/158918. In certain embodiments, a type V-A Cas protein comprises the amino acid sequence set forth in SEQ ID NO: 9 of International (PCT) Application Publication No. WO 2021/158918.

In certain embodiments, a type V-A Cas nuclease comprises Lachnospiraceae bacterium COE1 Cpf1 (Lb3Cpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 10 of International (PCT) Application Publication No. WO 2021158918. In certain embodiments, a type V-A Cas protein comprises the amino acid sequence set forth in SEQ ID NO: 10 of International (PCT) Application Publication No. WO 2021/158918.

In certain embodiments, a type V-A Cas nuclease comprises Eubacterium coprostanoligenes Cpf1 (EcCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 11 of International (PCT) Application Publication No. WO 2021158918. In certain embodiments, a type V-A Cas protein comprises the amino acid sequence set forth in SEQ ID NO: 11 of International (PCT) Application Publication No. WO 2021/158918.

In certain embodiments, a type V-A Cas nuclease is not Cpf1. In certain embodiments, a type V-A Cas nuclease is not AsCpf1.

In certain embodiments, a type V-A Cas nuclease comprises MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20, or variants thereof. MAD1-MAD20 are known in the art and are described in U.S. Pat. No. 9,982,279.

In certain embodiments, a type V-A Cas nuclease comprises MAD7 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 37. In certain embodiments, a type V-A Cas protein comprises the amino acid sequence set forth in SEQ ID NO: 37.

MAD7
(SEQ ID NO: 37)
MNNGTNNFQNFIGISSLQKTLRNALIPTETTQQFIVKNGIIKEDE
LRGENRQILKDIMDDYYRGFISETLSSIDDIDWTSLFEKMEIQLK
NGDNKDTLIKEQTEYRKAIHKKFANDDRFKNMFSAKLISDILPEF
VIHNNNYSASEKEEKTQVIKLESRFATSFKDYFKNRANCESADDI
SSSSCHRIVNDNAEIFFSNALVYRRIVKSLSNDDINKISGDMKDS
LKEMSLEEIYSYEKYGEFITQEGISFYNDICGKVNSFMNLYCQKN
KENKNLYKLQKLHKQILCIADTSYEVPYKFESDEEVYQSVNGELD
NISSKHIVERLRKIGDNYNGYNLDKIYIVSKFYESVSQKTYRDWE
TINTALEIHYNNILPGNGKSKADKVKKAVKNDLQKSITEINELVS
NYKLCSDDNIKAETYIHEISHILNNFEAQELKYNPEIHLVESELK
ASELKNVLDVIMNAFHWCSVFMTEELVDKDNNFYAELEEIYDEIY
PVISLYNLVRNYVTQKPYSTKKIKLNFGIPTLADGWSKSKEYSNN
AIILMRDNLYYLGIFNAKNKPDKKIIEGNTSENKGDYKKMIYNLL
PGPNKMIPKVFLSSKTGVETYKPSAYILEGYKQNKHIKSSKDFDI
TFCHDLIDYFKNCIAIHPEWKNFGFDFSDTSTYEDISGFYREVEL
QGYKIDWTYISEKDIDLLQEKGQLYLFQIYNKDFSKKSTGNDNLH
TMYLKNLFSEENLKDIVLKLNGEAEIFFRKSSIKNPIIHKKGSIL
VNRTYEAEEKDQFGNIQIVRKNIPENIYQELYKYFNDKSDKELSD
EAAKLKNVVGHHEAATNIVKDYRYTYDKYFLHMPITINFKANKTG
FINDRILQYIAKEKDLHVIGIDRGERNLIYVSVIDTCGNIVEQKS
FNIVNGYDYQIKLKQQEGARQIARKEWKEIGKIKEIKEGYLSLVI
HEISKMVIKYNAIIAMEDLSYGFKKGRFKVERQVYQKFETMLINK
LNYLVFKDISITENGGLLKGYQLTYIPDKLKNVGHQCGCIFYVPA
AYTSKIDPTTGFVNIFKFKDLTVDAKREFIKKFDSIRYDSEKNLF
CFTEDYNNFITQNTVMSKSSWSVYTYGVRIKRRFVNGRFSNESDT
IDITKDMEKTLEMTDINWRDGHDLRQDIIDYEIVQHIFEIFRLTV
QMRNSLSELEDRDYDRLISPVLNENNIFYDSAKAGDALPKDADAN
GAYCIALKGLYEIKQITENWKEDGKFSRDKLKISNKDWEDFIQNK
RYL

In certain embodiments, a type V-A Cas nuclease comprises MAD2 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 38. In certain embodiments, a type V-A Cas protein comprises the amino acid sequence set forth in SEQ ID NO: 38.

MAD2
(SEQ ID NO: 38)
MSSLTKFTNKYSKQLTIKNELIPVGKTLENIKENGLIDGDEQLNE
NYQKAKIIVDDELRDFINKALNNTQIGNWRELADALNKEDEDNIE
KLQDKIRGIIVSKFETFDLFSSYSIKKDEKIIDDDNDVEEEELDL
GKKTSSFKYIFKKNLFKLVLPSYLKTTNQDKLKIISSEDNESTYF
RGFFENRKNIFTKKPISTSIAYRIVHDNFPKFLDNIRCFNVWQTE
CPQLIVKADNYLKSKNVIAKDKSLANYFTVGAYDYFLSQNGIDFY
NNIIGGLPAFAGHEKIQGLNEFINQECQKDSELKSKLKNRHAFKM
AVLFKQILSDREKSFVIDEFESDAQVIDAVKNFYAEQCKDNNVIF
NLLNLIKNIAFLSDDELDGIFIEGKYLSSVSQKLYSDWSKLRNDI
EDSANSKQGNKELAKKIKTNKGDVEKAISKYEFSLSELNSIVHDN
TKFSDLLSCTLHKVASEKLVKVNEGDWPKHLKNNEEKQKIKEPLD
ALLEIYNTLLIFNCKSENKNGNFYVDYDRCINELSSVVYLYNKTR
NYCTKKPYNTDKFKLNENSPQLGEGFSKSKENDCLTLLFKKDDNY
YVGIIRKGAKINEDDTQAIADNTDNCIFKMNYFLLKDAKKFIPKC
SIQLKEVKAHFKKSEDDYILSDKEKFASPLVIKKSTFLLATAHVK
GKKGNIKKFQKEYSKENPTEYRNSLNEWIAFCKEFLKTYKAATIF
DITTLKKAEEYADIVEFYKDVDNLCYKLEFCPIKTSFIENLIDNG
DLYLFRINNKDESSKSTGTKNLHTLYLQAIFDERNLNNPTIMLNG
GAELFYRKESIEQKNRITHKAGSILVNKVCKDGTSLDDKIRNEIY
QYENKFIDTLSDEAKKVLPNVIKKEATHDITKDKRFTSDKFFFHC
PLTINYKEGDTKQFNNEVLSFLRGNPDINIIGIDRGERNLIYVTV
INQKGEILDSVSENTVINKSSKIEQTVDYEEKLAVREKERIEAKR
SWDSISKIATLKEGYLSAIVHEICLLMIKHNAIVVLENLNAGFKR
IRGGLSEKSVYQKFEKMLINKLNYFVSKKESDWNKPSGLLNGLQL
SDQFESFEKLGIQSGFIFYVPAAYTSKIDPTTGFANVLNLSKVRN
VDAIKSFFSNFNEISYSKKEALFKFSFDLDSLSKKGFSSFVKESK
SKWNVYTFGERIIKPKNKQGYREDKRINLTFEMKKLLNEYKVSED
LENNLIPNLTSANLKDTFWKELFFIFKTTLQLRNSVINGKEDVLI
SPVKNAKGEFFVSGTHNKTLPQDCDANGAYHIALKGLMILERNNL
VREEKDTKKIMAISNVDWFEYVQKRRGVL

In certain embodiments, a type V-A Cas nucleases comprises Csm1. Csm1 proteins are known in the art and are described in U.S. Pat. No. 9,896,696. Csm1 orthologs can be found in various bacterial and archaeal genomes. For example, in certain embodiments, a Csm1 protein is derived from Smithella sp. SCADC (Sm), Sulfuricurvum sp. (Ss), or Microgenomates (Roizmanbacteria) bacterium (Mb).

In certain embodiments, a type V-A Cas nuclease comprises SmCsm1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 12 of International (PCT) Application Publication No. WO 2021/158918. In certain embodiments, a type V-A Cas protein comprises the amino acid sequence set forth in SEQ ID NO: 12 of International (PCT) Application Publication No. WO 2021/158918.

In certain embodiments, a type V-A Cas nuclease comprises SsCsm1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 13 of International (PCT) Application Publication No. WO 2021/158918. In certain embodiments, a type V-A Cas protein comprises the amino acid sequence set forth in SEQ ID NO: 13 of International (PCT) Application Publication No. WO 2021/158918.

In certain embodiments, a type V-A Cas nuclease comprises MbCsm1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 14 of International (PCT) Application Publication No. WO 2021/158918. In certain embodiments, a type V-A Cas protein comprises the amino acid sequence set forth in SEQ ID NO: 14 of International (PCT) Application Publication No. WO 2021/158918.

In certain embodiments, the type V-A Cas nuclease comprises an ART nuclease or a variant thereof. In general, such nucleases sequences have <60% AA sequence similarity to Cas12a, <60% AA sequence similarity to a positive control nuclease, and >80% query cover. In certain embodiments, the Type V-A nuclease comprises an ART1, ART2, ART3, ART4, ART5, ART6, ART7, ART8, ART9, ART10, ART11, ART12, ART13, ART14, ART15, ART16, ART17, ART18, ART19, ART20, ART21, ART22, ART23, ART24, ART25, ART26, ART27, ART28, ART28, ART30, ART31, ART32, ART33, ART34, ART35, or ART11* (i.e., ART11_L679F, i.e., ART11 wherein leucine (L) at amino acid position 679 is replaced with phenylalanine (F)) nuclease, as shown in Table 3. In certain embodiments, the type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence designated for the individual ART nuclease as shown in Table 3. In certain embodiments, provided is a nucleic acid-guided nuclease comprising a nucleic acid-guided nuclease polypeptide having at least 85% identity to an amino acid sequence represented by SEQ ID NOs: 1-36 or a nucleic acid encoding a nucleic acid-guided nuclease polypeptide comprising at least 85% identity with the polynucleotide represented by SEQ ID NOs: 1-36. In certain embodiments, provided is a nucleic acid-guided nuclease comprising a polypeptide having at least 90% identity to the amino acid sequence represented by SEQ ID NOs: 1-36, wherein the polypeptide does not contain a peptide motif of YLFQIYNKDF (SEQ ID NO: 39). In certain embodiments, provided is a nucleic acid-guided nuclease comprising a nucleic acid encoding a polypeptide having at least 90% identity to nucleic acids represented by SEQ ID NOs: 808-845 wherein an encoded polypeptide does not contain a peptide motif of YLFQIYNKDF (SEQ ID NO: 39). In certain embodiments, provided is a nucleic acid-guided nuclease wherein the polypeptide comprises at least 90% identity with the amino acid sequence represented by SEQ ID NOs: 1-9. In certain embodiments, provided is a nucleic acid-guided nuclease, wherein the polypeptide comprises a polypeptide comprising at least 90% identity with the amino acid sequence represented by SEQ ID NO: 2, 11, or 36.

TABLE 3
ART nucleases

| | SEQ | |
|---|---|---|
| Name | ID NO | Amino Acid Sequence |
| ART1 | 1 | METFSGFTNLYPLSKTLRFRLIPVGETLKHFIDSGILEEDQHRAESYVK |
| | | VKAIIDDYHRAYIENSLSGFELPLESTKENSLEEYYLYHNIRNKTEEIQ |
| | | NLSSKVRTNLRKQVVAQLTKNEIFKRIDKKELIQSDLIDFVKNEPDANE |
| | | KIALISEFRNFTVYFKGFHENRRNMYSDEEKSTSIAFRLIHENLPKFID |
| | | NMEVFAKIQNTSISENFDAIQKELCPELVTLCEMEKLGYFNKTLSQKQI |
| | | DAYNTVIGGKTTSEGKKIKGLNEYINLYNQQHKQEKLPKMKLLFKQILS |
| | | DRESASWLPEKFENDSQVVGAIVNEWNTIHDTVLAEGGLKTIIASLGSY |
| | | GLEGIFLKNDLQLTDISQKATGSWGKISSEIKQKIEVMNPQKKKESYET |
| | | YQERIDKIFKSYKSFSLAFINECLRGEYKIEDYFLKLGAVNSSSLQKEN |
| | | HFSHILNTYTDVKEVIGLYSESTDTKLIQDNDSIQKIKQFLDAVKDLQA |
| | | YVKPLLGNGDETGKDERFYGDLIEYWSLLDLITPLYNMVRNYVTQKPYS |
| | | VDKIKINFQNPTLLNGWDLNKETDNTSVILRRDGKYYLAIMNNKSRKVF |
| | | LKYPSGTDRNCYEKMEYKLLPGANKMLPKVFFSKSRINEFMPNERLLSN |
| | | YEKGTHKKSGTCFSLDDCHTLIDFFKKSLDKHEDWKNFGFKFSDTSTYE |
| | | DMSGFYKEVENQGYKLSFKPIDATYVDQLVDEGKIFLFQIYNKDESEHS |
| | | KGTPNMHTLYWKMLFDETNLGDVVYKLNGEAEVFFRKASINVSHPTHPA |
| | | NIPIKKKNLKHKDEERILKYDLIKDKRYTVDQFQFHVPITMNFKADGNG |
| | | NINQKAIDYLRSASDTHIIGIDRGERNLLYLVVIDGNGKICEQFSLNEI |
| | | EVEYNGEKYSTNYHDLLNVKENERKQARQSWQSIANIKDLKEGYLSQVI |
| | | HKISELMVKYNAIVVLEDLNAGFMRGRQKVEKQVYQKFEKKLIEKLNYL |
| | | VFKKQSSDLPGGLMHAYQLANKFESFNTLGKQSGFLFYIPAWNTSKMDP |
| | | VTGFVNLFDVKYESVDKAKSFFSKEDSIRYNVERDMFEWKENYGEFTKK |
| | | AEGTKTDWTVCSYGNRIITFRNPDKNSQWDNKEINLTENIKLLFERFGI |
| | | DLSSNLKDEIMQRTEKEFFIELISLFKLVLQMRNSWTGTDIDYLVSPVC |
| | | NENGEFFDSRNVDETLPQNADANGAYNIARKGMILLDKIKKSNGEKKLA |
| | | LSITNREWLSFAQGCCKNG |
| ART2 | 2 | MISNFTNQYQLSKTERFELKPVGDTLKHIEKSGLIAQDEIRSQEYQEVK |
| | | TIIDKYHKAFIDEALQNVVLSNLEEYEALFFERNRDEKAFEKLQAVLRK |
| | | EIVAHFKQHPQYKTLFKKELIKADLKNWQELSDAEKELVSHEDNFTTYF |
| | | TGEHENRANMYTDEAKHSSIAYRIIHENLPIFLINKKLFETIKQKAPHL |
| | | AQETQDALLEYLSGAIVEDMFELSYENHELSQTHIDLYNQMIGGVKQDS |
| | | IKIQGLNEKINLYRQANGLSKRELPNLKPLHKQILSDRETLSWLPESFE |
| | | SDEELMQGVQAYFESEVLAFECCDGKVNLLEKLPELLHQTQDYDESKVY |
| | | FKNDLALTAASQAIFKDYRIIKEALWEVNKPKKSKDLVADEEKFENKKN |
| | | SYFSIEQIDGALNSAQLSANMMHYFQSESTKVIEQIQLTYNDWKRNSSN |
| | | KELLKAFLDALLSYQRLLKPLNAPNDLEKDVAFYAYFDAYFTSLCGVVK |
| | | LYDKVRNEMIKKPYSLEKFKLNFENSTLLDGWDVNKESDNTAILFRKEG |
| | | LYYLGIMNKKYNKVERNISSSQDEGYQKIDYKLLPGANKMLPKVFFSDK |
| | | NKEYFKPNAKLLERYKAGEHKKGDNFDLDFCHELIDFFKTSIEKHQDWK |
| | | HFAYQFSPTESYEDLSGFYREVEQQGYKISYKNIAASFIDILVAEGKLY |
| | | FFQIYNKDESPYSKGTPNMHTLYWRALFDEKNLADVIYKINGQAEIFER |
| | | KKSIEYSQEKLQKGHHHEMLKDKFAYPIIKDRRFAFDKFQFHVPITINF |
| | | KAEGNENITPKTFEYIRSNPDNIKVIGIDRGERHLLYLSLIDAEGKIVE |
| | | QFTLNQIINSYNGKDHVIDYHAKLDAKEKDRDKARKEWGTVENIKELKE |
| | | GYLSHVIHKIATLIIEHGAVVAMEDLNFGFKRGRFKVEKQVYQKFEKAL |
| | | IDKLNYLVDKKKEPHKLGGLINALQLTSKFQSFEKMGKQNGELFYVPAW |
| | | NTSKIDPVTGFVNLFDTRYASVEKSKAFFTKFQSICYNEAKDYFELVED |
| | | YNDFTEKAKETRSEWTLCTYGERIVSFRNAEKNHQWDSKTIHLTTEFKN |
| | | LFGELHGNDVKEYILEQNSVEFFKSLIYLLKITLQMRNSITGTDIDYLV |
| | | SPVADEAGNFYDSRKADTSLPKDADANGAYNIARKGIMIMHRIQNAEDL |
| | | KKVNLAISNRDWLRNAQGLDK |
| ART3 | 3 | MIDLKQFIGIYPVSKTLRFELRPVGKTQEWIEKNRVLEGDEQKAADYPV |
| | | VKKLIDDYHKVCIHDSLNHVHEDWEPLKDAIEIFQKTKSDEAKKRLEAE |
| | | QAMMRKKIAAAIKDFKHFKELTAATPSDLITSVLPEFSDDGSLKSERGE |
| | | ATYFSGFQENRNNIYSQEAISTGVPYRLVHDNFPKFLSDLEVFERIKST |
| | | CPEVINQASAELQPFLEGVMIDDIFSLDFYNSLLTQNGIDFFNQVIGGV |
| | | SEKDKQKYRGINEFSNLYRQQHKEIAASKKAMTMIPLFKQILSDRDTLS |
| | | YIPAQIRTEDELVSSITQFYDHITHFEHDGKTINVLSEIVALLGKLDTY |
| | | DPNGICITARKLTDISQKVYGKWSVIEEKMKEKAIQQYGDISVAKNKKK |
| | | VDAFLSRKAYSLSDLCFDEEISFSRYYSELPQTLNAISGYWLQFNEWCK |
| | | SDEKQKFLNNQTGTEVVKSLLDAMMELFHKCSVLVMPEEYEVDKSFYNE |
| | | FLPLYEELDTLFLLYNKVRNYLTQKPSDVKKFKLNFESPSLASGWDQNK |
| | | EMKNNAILLFKDGKSYLGVLNAKNKAKIKDAKGDVSSSSYKKMIYKLLS |
| | | DPSKDLPHKIFAKGNLDFYKPSEYILEGRELGKYKKGPNFDKKFLHDFI |
| | | DFYKAAISIDPDWSKFNFQYSPTESYDDIGMFFSEIKKQAYKIRFTDIS |
| | | EAQVNEWVDNGQLYLFQLYNKDYAEGAHGRKNLHTLYWENLFTDENLSN |
| | | LVLKLNGQAELFCRPQSIKKPVSHKIGSKMLNRRDKSGMPIPESIYRSL |
| | | YQYYNGKKKESELTVAEKQYIDQVIVKDVTHEIIKDRRYTRQEYFFHVP |
| | | LTFNANADGNEYINEHVLNYLKDNPDVNIIGIDRGERHLIYLTLINQRG |
| | | EILKQKTFNVVNSYNYQAKLEQREKERDEARKSWDSVGKIKDLKEGELS |
| | | AVIHEITNMMIENNAIVVLEDLNFGFKRGRFKVERQVYQKFEKMLIDKL |
| | | NYLSFKDREAGEEGGILRGYQMAQKFISFQRLGKQSGFLFYIPAAYTSK |
| | | IDPVSGFVNHFNFSDITNAEKRKDFLMKMDRIEMKNGNIEFTFDYRKEK |
| | | TFQTDYQNVWTVSTFGKRIVMRIDEKGYKKMVDYEPTNDIIKAFKNKGI |
| | | LLSEGSDLKALIAEIEANATNAGFYSTLLYAFQKTLQMRNSNAVTEEDY |
| | | ILSPVAKDGHQFCSTDEANKGKDAQGNWVSKLPVDADANGAYHIALKGL |
| | | YLLRNPETKKIENEKWLQFMVEKPYLE |
| ART4 | 4 | MSYNREKMEEKELGKNQNFQEFIGVSPLQKTLRNELIPTETTKKNIAQL |
| | | DLLTEDEVRAQNREKLKEMMDDYYRDVIDSTLRGELLIDWSYLFSCMRN |
| | | HLSENSKESKRELERTQDSVRSQIHDKFAERADEKDMFGASIITKLLPT |
| | | YIKQNSKYSERYDESVKIMKLYGKFTTSLTDYFETRKNIFSKEKISSAV |
| | | GYRIVEENAEIFLQNQNAYDRICKIAGLDLHGLDNEITAYVDGKTLKEV |
| | | CSDEGFAKVITQGGIDRYNEAIGAVNQYMNLLCQKNKALKPGQFKMKRL |
| | | HKQILCKGTTSFDIPKKFENDKQVYDAVNSFTEIVTKNNDLKRLLNITQ |
| | | NANDYDMNKIYVVADAYSMISQFISKKWNLIEECLLDYYSDNLPGKGNA |
| | | KENKVKKAVKEETYRSVSQLNEVIEKYYVEKTGQSVWKVESYISSLAEM |
| | | IKLELCHEIDNDEKHNLIEDDEKISEIKELLDMYMDVFHIIKVERVNEV |
| | | LNFDETFYSEMDEIYQDMQEIVPLYNHVRNYVTQKPYKQEKYRLYFHTP |
| | | TLANGWSKSKEYDNNAIILVREDKYYLGILNAKKKPSKEIMAGKEDCSE |
| | | HAYAKMNYYLLPGANKMLPKVELSKKGIQDYHPSSYIVEGYNEKKHIKG |
| | | SKNFDIRFCRDLIDYFKECIKKHPDWNKENFEFSATETYEDISVFYREV |
| | | EKQGYRVEWTYINSEDIQKLEEDGQLFLFQIYNKDFAVGSTGKPNLHTL |
| | | YLKNLFSEENLRDIVLKLNGEAEIFFRKSSVQKPVIHKCGSILVNRTYE |
| | | ITESGTTRVQSIPESEYMELYRYFNSEKQIELSDEAKKYLDKVQCNKAK |
| | | TDIVKDYRYTMDKFFIHLPITINFKVDKGNNVNAIAQQYIAEQEDLHVI |
| | | GIDRGERNLIYVSVIDMYGRILEQKSFNLVEQVSSQGTKRYYDYKEKLQ |
| | | NREEERDKARKSWKTIGKIKELKEGYLSSVIHEIAQMVVKYNAIIAMED |
| | | LNYGFKRGRFKVERQVYQKFETMLISKLNYLADKSQAVDEPGGILRGYQ |
| | | MTYVPDNIKNVGRQCGIIFYVPAAYTSKIDPTTGFINAFKRDVVSTNDA |
| | | KENFLMKFDSIQYDIEKGLFKFSFDYKNFATHKLTLAKTKWDVYINGTR |
| | | IQNMKVEGHWLSMEVELTTKMKELLDDSHIPYEEGQNILDDLREMKDIT |
| | | TIVNGILEIFWLTVQLRNSRIDNPDYDRIISPVLNNDGEFFDSDEYNSY |
| | | IDAQKAPLPIDADANGAFCIALKGMYTANQIKENWVEGEKLPADCLKIE |
| | | HASWLAFMQGERG |
| ART5 | 5 | MSAVFKIKESTMKDFTHQYSLSKTLRFELKPVGETAERIEDFKNQGLKS |
| | | IVEEDRQRAEDYKKMKRILDDYHKEFIEEVLNDDIFTANEMESAFEVYR |
| | | KYMASKNDDKLKKEITEIFTDLRKKIAKAFENKSKEYCLYKGDESKLIN |
| | | EKKTGKDKGPGKLWYWLKAKADAGVNEFGDGQTFEQAEEALAKENNEST |
| | | YFTGFNQNRDNIYTDAEQQTAISYRVINENMTRYFDNCIRYSSIENKYP |
| | | ELVKQLEPLSGKFAPGNYKDYLSQTAIDIYNEAVGHKSDDINAKGINQF |
| | | INEYRQRNSIKGRELPIMSVLYKQILSDINKDLIIDKFENAGELLDAVK |
| | | TLHRELTDKKILLKIKQTLNEFLTEDNSEDIYIKSGTDLTAVSNAIWGE |
| | | WSVIPKALEMYAENITDMNAKAREKWLKREAYHLKTVQEAIEAYLKDNE |
| | | EFETRNISEYFTNFKSGENDLIQVVQSAYAKMESIFGIEDFHKDRRPVT |
| | | ESGEPGEGFRQVELVREYLDSLINVEHFIKPLHMERSGKPIELEDCNSN |
| | | FYDPLNEAYKELDVVFGIYNKVRNYVTQKPYSKDKFKINFQNSTLLDGW |
| | | DVNKESANSSVLLLKNGKYYLGVMKQGASNILNYRPEPSDSKNKINAKK |
| | | QLSEIALAGATDDYYEKMIYKLLPDPAKMLPKVFFSAKNIEFYNPSQEI |
| | | IYIRENGLFKKDAGDKESLKKWIGFMKTSLLKHPEWGSYFNFEFEPAED |
| | | YQDISIFYKQVAEQGYSVTFDKIKTSYIEEKVASGELYLFEIYNKDFSP |
| | | HSKGRPNLHTMYWKSLFEKENLQNLVTKLNGEAEVFFRQHSIKRNEKVV |
| | | HRANRPIQNKNPLTEKKQSIFEYDLVKDRRFTKDKFFLHCPITLNFKEA |
| | | GPGRFNDKVNKYIAGNPDIRIIGIDRGERHLLYYSLIDQSGRIVEQGTL |
| | | NQITSTLNSGGREIPKTTDYRGLLDTKEKERDKARKSWSMIENIKELKS |
| | | GYLSHIVHKLAKLMVKNNAVVVLEDLNFGFKRGRFKVEKQVYQKFEKAL |
| | | IEKLNYLVFKDARPAEPGHYLNAYQLTAPLESFKKLGKQSGFIYYVPAW |
| | | NTSKIDPVTGFVNQFYIEKNSMQYLKNFFGKFDSIRFNPDKNYFEFGFD |
| | | YKNFHNKAAKSKWTICTHGDKRSWYNRKQRKLEIHNVTENLASLLSGKG |
| | | INFADGGSIKDKILSVDDASFFKSLAFNFKLTAQLRHTFEDNGEEIDCI |
| | | ISPVAAADGTFFCSETAKKLNMELPHDADANGAYNIARKGLMVLRQIRE |
| | | SGKPKPISNADWLDFAQQNED |
| ART6 | 6 | MQERKKISHLTHRNSVQKTIRMQLNPVGKTMDYFQAKQILENDEKLKEN |
| | | YQKIKEIADRFYRNLNEDVLSKTGLDKLKDYAEIYYHCNTDAERKRLDE |
| | | CASELRKEIVKNFKNRDEYNKLENKKMIEIVLPQHLKNEDEKEVVASEK |
| | | NFTTYFTGFFTNRKNMYSDGEESTAIAYRCINENLPKHLDNVKAFEKAI |
| | | SKLSKNAIDDLDATYSGLCGTNLYDVFTVDYFNFLLPQSGITEYNKIIG |
| | | GYTTSDGTKVKGINEYINLYNQQVSKRDKIPNLKILYKQILSESEKVSF |
| | | IPPKFEDDNELLSAVSEFYANDETFDGMPLKKAIDETKLLFGNLDNSSL |
| | | NGIYIQNDRSVTNLSNSMFGSWSVIEDLWNKNYDSVNSNSRIKDIQKRE |
| | | DKRKKAYKAEKKLSLSFLQVLISNSENDEIREKSIVNYYKTSLMQLTDN |
| | | LSDKYNEAAPLLNKSYANEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDRNKEKDCGAVWLCRDEKYYLAIIDKSNNSILENIDE |
| | | QDCDENDCYEKIIYKLLPGPNKMLPKVFFSEKCKKLLSPSDEILKIRKN |
| | | GTFKKGDKFSLDDCHKLIDFYKESFKKYPNWLIYNFKFKNTNEYNDIRE |
| | | FYNDVASQGYNISKMKIPTSFIDKLVDEGKIYLFQLYNKDFSPHSKGTP |
| | | NLHTLYFKMLFDERNLEDVVYKLNGEAEMFYRPASIKYDKPTHPKNTPI |
| | | KNKNTLNDKKTSTFPYDLIKDKRYTKWQFSLHFPITMNFKAPDRAMIND |
| | | DVRNLLKSCNNNFIIGIDRGERNLLYVSVIDSNGAIIYQHSLNIIGNKE |
| | | KGKTYETNYREKLATREKERTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEGGGLLHAYQLTNKLESFDKLGMQSGFIFYVRPDFTSKIDPVTGF |
| | | VNLLYPRYENIDKAKDMISREDDIGYNAGEDFFEFDIDYDKFPKTASDY |
| | | RKRWTICINGERIEAFRNPAKNNEWSYRTIILAEKFKELFDNNSINYRD |
| | | SDDLKAEILSQTKGKFFEDFFKLLRLTLQMRNSNPETGEDRILSPVKDK |
| | | NGNFYDSSKYDEKSKLPCDADANGAYNIARKGLWIVEQFKKSDNVSTVG |
| | | PVIHNDKWLKFVQENDMANN |
| ART7 | 7 | MNILKENYMKEIKELTGLYSLTKTIGVELKPVGKTQELIEAKKLIEQDD |
| | | QRAEDYKIVKDIIDRYHKDFIDKCLNCVKIKKDDLEKYVSLAENSNRDA |
| | | EDFDKIKTKMRNQITEAFRKNSLFTNLFKKNLIKEYLPAFVSEEEKSVV |
| | | NKFSKFTTYFDAFNDNRKNLYSGDAKSGTIAYRLIHENLPMELDNIASF |
| | | NAISGIGVNEYFSSIETEFTDTLEGKRLTEFFQIDFENNTLTQKKIGNY |
| | | NYIVGAVNKAVNLYKQQHKTVRVPLLKPLYKMILSDRVTPSWLPERFES |
| | | DEEMLTAIKAAYESLREVLVGDNDESLRNLLLNIEHYDLEHIYIANDSG |
| | | LTSISQKIFGCYDTYTLAIKDQLQRDYPATKKQREAPDLYDERIDKLYK |
| | | KVGSFSIAYLNRLVDAKGHFTINEYYKQLGAYCREEGKEKDDFFKRIDG |
| | | AYCAISHLFFGEHGEIAQSDSDVELIQKLLEAYKGLQRFIKPLLGHGDE |
| | | ADKDNEFDAKLRKVWDELDIITPLYDKVRNWLSRKIYNPEKIKLCFENN |
| | | GKLLSGWVDSRTKSDNGTQYGGYIFRKKNEIGEYDFYLGISADTKLERR |
| | | DAAISYDDGMYERLDYYQLKSKTLLGNSYVGDYGLDSMNLLSAFKNAAV |
| | | KFQFEKEVVPKDKENVPKYLKRLKLDYAGFYQILMNDDKVVDAYKIMKQ |
| | | HILATLTSSIRVPAAIELATQKELGIDELIDEIMNLPSKSFGYFPIVTA |
| | | AIEEANKRENKPLFLFKMSNKDLSYAATASKGLRKGRGTENLHSMYLKA |
| | | LLGMTQSVEDIGSGMVFFRHQTKGLAETTARHKANEFVANKNKLNDKKK |
| | | SIFGYEIVKNKRFTVDKYLFKLSMNLNYSQPNNNKIDVNSKVREIISNG |
| | | GIKNIIGIDRGERNLLYLSLIDLKGNIVMQKSLNILKDDHNAKETDYKG |
| | | LLTEREGENKEARRNWKKIANIKDLKRGYLSQVVHIISKMMVEYNAIVV |
| | | LEDLNPGFIRGRQKIERNVYEQFERMLIDKLNFYVDKHKGANETGGLLH |
| | | ALQLTSEFKNFKKSEHQNGCLFYIPAWNTSKIDPATGFVNLENTKYTNA |
| | | VEAQEFFSKFDEIRYNEEKDWFEFEFDYDKFTQKAHGTRTKWTLCTYGM |
| | | RLRSFKNSAKQYNWDSEVVALTEEFKRILGEAGIDIHENLKDAICNLEG |
| | | KSQKYLEPLMQFMKLLLQLRNSKAGTDEDYILSPVADENGIFYDSRSCG |
| | | DQLPENADANGAYNIARKGLMLIEQIKNAEDLNNVKFDISNKAWINFAQ |
| | | QKPYKNG |
| ART8 | 8 | MAKENIFNELTGKYQLSKTLRLELKPVGNTQQMLKDEDVFEKDRIIREK |
| | | YRETRPHFDRLHREFIEQALKNQKLSDLGKYFQCLAKLQNNKKDKEAQE |
| | | EFKRISQNLRKEVNDLFKIDPLFGEGVFALLKEKYGEKDDAFLREQDGQ |
| | | YVLDENKKKISIFDSWKGFTGYFTKFQETRKNFYKDDGTATAVATRIID |
| | | QNLKRFCENIQIFKSIQKKVDFKEVEDNFSVDLEDIFSLGFYSSCELQE |
| | | GIDVYNKILGGEPKTTGEKLRGLNELINRYRQDHKGEKLPFFKMLDKQI |
| | | LSEKEKFIESIEDDEELLKTLKEFYSSAEEKTTVLKELENDFIKNNENY |
| | | DLSEIYISREALNTISHRWVSAATLPEFEKSVYEVMKKDKPSGLSFDKD |
| | | DNSYKFPDFIALSYIKGSFEKLSGEKLWKDGYFRDETRNGDKGFLIGNE |
| | | SLWTQFIKIFEFEFNSLFEAKNTERSVGYYHFKKDFEKIITNDESVNPE |
| | | DKVIIREFADNVLAIYQMAKYFAIEKKRKWMDQYDTGDFYNHPDFGYKT |
| | | KFYDNAYEKIVKARMLLQSYLTKKPFSTDKWKLNFECGYLLNGWSSSEN |
| | | TYGSLLFRTGNEYYLGVVNGSALRTEKIKRLIGNITEANSCHKMVYDFQ |
| | | KPDNKNVPRIFIRSKGDKFAPAVSELNLPVDSILEIYDKGLFKTENKNS |
| | | PFFKPSLKKLIDYFKLGFSRHASYKHYQFKWKDSSEYKNISEFYNDTIR |
| | | SCYQIKWEELNFEEVKKLINSKDLFLFQIYNKDFSEKSTGNKNLHSIYF |
| | | DGLFLDNNINAQDGVILKLSGGGEIFFRPKTDVKKLGSRTDTKGKLVIK |
| | | NKRYSQDKIFLHFPIELNYSNTQESNFNKLVRNFLADNPDINIIGVDRG |
| | | EKHLIYYAGIDQKGNTLKDKDDKDVLGSLNEINGVNYYKLLEERAKARE |
| | | KARQDWQNIQGIKDLKMGYISLVVRKLADLIIEYNAILVLEDLNMRFKQ |
| | | IHGGIEKSVYQQLEKALIEKLNFLVNKGEKDPERAGHLLRAYQLTAPES |
| | | TFKDMGKQTGVLFYTQASYTSKTCPQCGFRPNIKLHFDNLENAKKMLEK |
| | | INIVYKDNHFEIGYKVSDFTKTEKTSRGNILYGDRQGKDTFVISSKAAI |
| | | RYKWFARNIKNNELNRGESLKEHTEKGVTIQYDITECLKILYEKNGIDH |
| | | SGDITKQSIRSELPAKFYKDLLFYLYLLTNTRSSISGTEIDYINCPDCG |
| | | FHSEKGFNGCIFNGDANGAYNIARKGMLILKKINQYKDQHHTMDKMGWG |
| | | DLFIGIEEWDKYTQVVSRS |
| ART9 | 9 | MKEIKELTGLYSLTKTIGVELKPVGKTQELIEAKKLIEQDDQRAEDYKI |
| | | VKDIIDRYHKDFIDKCLNCVKIKKDDLEKYVSLAENSNRDAEDEDKIKT |
| | | KMRNQITEAFRKNSLFTNLFKKNLIKEYLPAFVSEEEKSVVNKFSKFTT |
| | | YFDAFNDNRKNLYSGDAKSGTIAYRLIHENLPMFLDNIASFNAISGIGV |
| | | NEYFSSIETEFTDTLEGKRLTEFFQIDFFNNTLTQKKIGNYNYIVGAVN |
| | | KAVNLYKQQHKTVRVPLLKPLYKMILSDRVTPSWLPERFESDEEMLTAI |
| | | KAAYESLREVLVGDNDESLRNLLLNIEHYDLEHIYIANDSGLTSISQKI |
| | | FGCYDTYTLAIKDQLQRDYPATKKQREAPDLYDERIDKLYKKVGSFSIA |
| | | YLNRLVDAKGHFTINEYYKQLGAYCREEGKEKDDFFKRIDGAYCAISHL |
| | | FFGEHGEIAQSDSDVELIQKLLEAYKGLQRFIKPLLGHGDEADKDNEED |
| | | AKLRKVWDELDIITPLYDKVRNWLSRKIYNPEKIKLCFENNGKLLSGWV |
| | | DSRTKSDNGTQYGGYIFRKKNEIGEYDFYLGISADTKLFRRDAAISYDD |
| | | GMYERLDYYQLKSKTLLGNSYVGDYGLDSMNLLSAFKNAAVKFQFEKEV |
| | | VPKDKENVPKYLKRLKLDYAGFYQILMNDDKVVDAYKIMKQHILATLTS |
| | | SIRVPAAIELATQKELGIDELIDEIMNLPSKSFGYFPIVTAAIEEANKR |
| | | ENKPLFLFKMSNKDLSYAATASKGLRKGRGTENLHSMYLKALLGMTQSV |
| | | FDIGSGMVFFRHQTKGLAETTARHKANEFVANKNKLNDKKKSIFGYEIV |
| | | KNKRFTVDKYLFKLSMNLNYSQPNNNKIDVNSKVREIISNGGIKNIIGI |
| | | DRGERNLLYLSLIDLKGNIVMQKSLNILKDDHNAKETDYKGLLTEREGE |
| | | NKEARRNWKKIANIKDLKRGYLSQVVHIISKMMVEYNAIVVLEDLNPGF |
| | | IRGRQKIERNVYEQFERMLIDKLNFYVDKHKGANETGGLLHALQLTSEF |
| | | KNFKKSEHQNGCLFYIPAWNTSKIDPATGFVNLENTKYTNAVEAQEFFS |
| | | KFDEIRYNEEKDWFEFEFDYDKFTQKAHGTRTKWTLCTYGMRLRSFKNS |
| | | AKQYNWDSEVVALTEEFKRILGEAGIDIHENLKDAICNLEGKSQKYLEP |
| | | LMQFMKLLLQLRNSKAGTDEDYILSPVADENGIFYDSRSCGDQLPENAD |
| | | ANGAYNIARKGLMLIEQIKNAEDLNNVKFDISNKAWLNFAQQKPYKNG |
| ART10 | 10 | MNFQPFFQKFVHLYPISKTLRFELIPQGATQKFISEKQVLLQDEIRARK |
| | | YPEMKQAIDGYHKDFIQRALSNIDSQVFEQALNTFEDLFLRSQAERATD |
| | | AYKKDFETAQTKLRELIVHSFEKGEFKQEYKSLFDKNLITNLLKPWVEQ |
| | | QNQIGDSNYTYHEDENKFTTYFLGFHENRKNIYSKDPHKTALAYRLIHE |
| | | NLPKFLENNKILLKIQNDHPSLWEQLQTLNQTMPQLFDGWDFSQLMQVS |
| | | FFSNTLTQTGIDQYNTIIGGISEGENRQKIQGINELINLYNQKQDKKNR |
| | | VAKLKQLYKQILSDRSTLSFLPEKFVDDTELYHAINMFYLEHLHHQSMI |
| | | NGHSYTLLERVQLLINELANYDLSKVYLAPNQLSTVSHQMFGDFGYIGR |
| | | ALNYYYMQVIQPDYEQLLASAKTTKKIEATEKLKTIFLDTPQSLVVIQA |
| | | AIDEYIQLQPSTKPHTQLTDFIISLLKQYETVADDQSIKVINVESDIEG |
| | | KYSCIKGLVNTKSESKREVLQDEKLATDIKAFMDAVNNVIKLLKPFSLN |
| | | EKLVASVEKDARFYSDFEEIYQSLLIFVPLYNKVRNYITQKPYSTEKFK |
| | | LNFNKPTLLSGWDANKEADNLSILLRKNGNYYLAIMDTAKGANKAFEPK |
| | | TLNQLKVDDTTDCYEKMVYKLLSGPSKMFPKAFKAKNNEGNYYPTPELL |
| | | TSYNNNEHLKNDKNFTLASLHAYIDWCKEYINRNPSWHQFNFKESPTQS |
| | | FQDISQFYSEVSSQSYKVHFQTIPSDYIDQLVAEGKLYLFQIYNKDFSP |
| | | NAKGKENLHTLYFKALFSDENLKQPVFKLSGEAEMFYRPASLQLANTTI |
| | | HKAGEPMAAKNPLTPNATRTLAYDIIKDRRFTTDKYLLHVPISLNFHAQ |
| | | ESMSIKKHNDLVRQMIKHNHQDLHVIGIDRGEKHLLYVSVIDLKGNIVY |
| | | QESLNSIKSEAQNFETPYHQLLQHREEGRAQARTAWGKIENIKELKDGY |
| | | LSQVVHRIQQLILKYNAIVMLEDLNFGFKRGRFKIEKQIYQKFEKALIH |
| | | KLNYVVDKSTQADELGGVRKAYQLTAPFESFEKLGKQSGVLFYVPAWNT |
| | | SKIDPVTGFVDLLKPKYENLDKAQAFFNAFDSIHYNAQKNYFEFKVNLK |
| | | QFAGLKAQAAQAEWTICSYGDERHVYQKKNAQQGETVIVNVTEELKVLF |
| | | AKNNIEVAQSVELKETICTQTQVDFFKRLMWLLQVLLALRYSSSKDKLD |
| | | YILSPVANAQGEFFDSRHASVQLPQDSDANGAYHIALKGLWVIEQLKAA |
| | | DNLDKVKLAISNDDWLHFAQQKPYLA |
| ART11 | 11 | MYYQGLTKLYPISKTIRNELIPVGKTLEHIRMNNILEADIQRKSDYERV |
| | | KKLMDDYHKQLINESLQDVHLSYVEEAADLYLNASKDKDIVDKFSKCQD |
| | | KLRKEIVNLLKSHENFPKIGNKEIIKLLQSLSDTEKDYNALDSFSKFYT |
| | | YFTSYNEVRKNLYSDEEKSSTAAYRLINENLPKELDNIKAYSIAKSAGV |
| | | RAKELTEEEQDCLEMTETFERTLTQDGIDNYNELIGKLNFAINLYNQQN |
| | | NKLKGFRKVPKMKELYKQILSEREASFVDEFVDDEALLTNVESFSAHIK |
| | | EFLESDSLSRFAEVLEESGGEMVYIKNDTSKTTFSNIVFGSWNVIDERL |
| | | AEEYDSANSKKKKDEKYYDKRHKELKKNKSYSVEKIVSLSTETEDVIGK |
| | | YIEKLQADIIAIKETREVFEKVVLKEHDKNKSLRKNTKAIEAIKSFLDT |
| | | IKDFERDIKLISGSEHEMEKNLAVYAEQENILSSIRNVDSLYNMSRNYL |
| | | TQKPFSTEKFKLNFNRATLLNGWDKNKETDNLGILLVKEGKYYLGIMNT |
| | | KANKSFVNPPKPKTDNVYHKVNYKLLPGPNKMLPKVFFAKSNLEYYKPS |
| | | EDLLAKYQAGTHKKGENFSLEDCHSLISFFKDSLEKHPDWSEFGFKFSD |
| | | TKKYDDLSGFYREVEKQGYKITYTDIDVEYIDSLVEKDELYLFQIYNKD |
| | | FSPYSKGNYNLHTLYLTMLFDERNLRNVVYKLNGEAEVFYRPASIGKDE |
| | | LIIHKSGEEIKNKNPKRAIDKPTSTFEYDIVKDRRYTKDKFMLHIPVTM |
| | | NFGVDETRRFNEVVNDAIRGDDKVRVIGIDRGERNLLYVVVVDSDGTIL |
| | | EQISLNSIINNEYSIETDYHKLLDEKEGDRDRARKNWTTIENIKELKEG |
| | | YLSQVVNVIAKLVLKYDAIICLEDLNFGFKRGRQKVEKQVYQKFEKMLI |
| | | DKLNYLVIDKSRSQENPEEVGHVLNALQLTSKFTSFKELGKQTGIIYYV |
| | | PAYLTSKIDPTTGFANLFYVKYESVEKSKDFFNREDSICENKVAGYFEF |
| | | SFDYKNFTDRACGMRSKWKVCTNGERIIKYRNEEKNSSFDDKVIVLTEE |
| | | FKKLFNEYGIAFNDCMDLTDAINAIDDASFFRKLTKLFQQTLQMRNSSA |
| | | DGSRDYIISPVENDNGEFFNSEKCDKSKPKDADANGAFNIARKGLWVLE |
| | | QLYNSSSGEKLNLAMTNAEWLEYAQQHTI |
| ART12 | 12 | MAKNFEDFKRLYPLSKTLRFEAKPIGATLDNIVKSGLLEEDEHRAASYV |
| | | KVKKLIDEYHKVFIDRVLDNGCLPLDDKGDNNSLAEYYESYVSKAQDED |
| | | AIKKFKEIQQNLLSIIAKKLTDDKAYANLFGNKLIESYKDKADKTKLID |
| | | SDLIQFINTAESTQLVSMSQDEAKELVKEFWGFTTYFEGFFKNRKNMYT |
| | | PEEKSTGIAYRLINENLPKFIDNMEAFKKAIARPEIQANMEELYSNFSE |
| | | YLNVESIQEMFLLDYYNMLLTQKQIDVYNAIIGGKTDDEHDVKIKGINE |
| | | YINLYNQQHKDDKLPKLKALFKQILSDRNAISWLPEEFNSDQEVLNAIK |
| | | DCYERLAENVLGDKVLKSLLGSLADYSLDGIFIRNDLQLTDISQKMEGN |
| | | WGVIQNAIMQNIKHVAPARKHKESEEDYEKRIAGIFKKADSFSISYIND |
| | | CLNEADPNNAYFVENYFATFGAVNTPTMQRENLFALVQNAYTEVAALLH |
| | | SDYPTVKHLAQDKANVSKIKALLDAIKSLQHFVKPLLGKGDESDKDERF |
| | | YGELASLWAELDTVTPLYNMIRNYMTRKPYSQKKIKLNFENPQLLGGWD |
| | | ANKEKDYATIILRRNGLYYLAIMDKDSRKLLGKAMPSDGECYEKMVYKE |
| | | FKDVTTMIPKCSTQLKDVQAYFKVNTDDYVLNSKAFNRPLTITKEVEDL |
| | | NNVLYGKYKKFQKGYLTATGDNVGYTHAVNVWIKFCMDFLDSYDSTCIY |
| | | DESSLKPESYLSLDSFYQDVNLLLYKLSFTDVSASFIDQLVEEGKMYLF |
| | | QIYNKDFSEYSKGTPNMHTLYWKALFDERNLADVVYKLNGQAEMFYRKK |
| | | SIENTHPTHPANHPILNKNKDNKKKESLFEYDLIKDRRYTVDKFMFHVP |
| | | ITMNFKSSGSENINQDVKAYLRHADDMHIIGIDRGERHLLYLVVIDLQG |
| | | NIKEQFSLNEIVNDYNGNTYHTNYHDLLDVREDERLKARQSWQTIENIK |
| | | ELKEGYLSQVIHKITQLMVRYHAIVVLEDLSKGFMRSRQKVEKQVYQKF |
| | | EKMLIDKLNYLVDKKTDVSTPGGLLNAYQLTCKSDSSQKLGKQSGFLFY |
| | | IPAWNTSKIDPVTGFVNLLDTHSLNSKEKIKAFFSKFDAIRYNKDKKWF |
| | | EFNLDYDKFGKKAEDTRTKWTLCTRGMRIDTERNKEKNSQWDNQEVDLT |
| | | TEMKSLLEHYYIDIHGNLKDAISTQTDKAFFTGLLHILKLTLQMRNSIT |
| | | GTETDYLVSPVADENGIFYDSRSCGDQLPENADANGAYNIARKGLMLVE |
| | | QIKDAEDLDNVKFDISNKAWLNFAQQKPYKNG |
| ART13 | 13 | MAKNFEDFKRLYSLSKTLRFEAKPIGATLDNIVKSGLLDEDEHRAASYV |
| | | KVKKLIDEYHKVFIDRVLDDGCLPLENKGNNNSLAEYYESYVSRAQDED |
| | | AKKKFKEIQQNLRSVIAKKLTEDKAYANLFGNKLIESYKDKEDKKKIID |
| | | SDLIQFINTAESTQLDSMSQDEAKELVKEFWGFVTYFYGFFDNRKNMYT |
| | | AEEKSTGIAYRLVNENLPKFIDNIEAFNRAITRPEIQENMGVLYSDESE |
| | | YLNVESIQEMFQLDYYNMLLTQKQIDVYNAIIGGKTDDEHDVKIKGINE |
| | | YINLYNQQHKDDKLPKLKALFKQILSDRNAISWLPEEFNSDQEVLNAIK |
| | | DCYERLAENVLGDKVLKSLLGSLADYSLDGIFIRNDLQLTDISQKMEGN |
| | | WGVIQNAIMQNIKRVAPARKHKESEEDYEKRIAGIFKKADSFSISYIND |
| | | CLNEADPNNAYFVENYFATFGAVNTPTMQRENLFALVQNAYTEVAALLH |
| | | SDYPTVKHLAQDKANVSKIKALLDAIKSLQHFVKPLLGKGDESDKDERF |
| | | YGELASLWAELDTVTPLYNMIRNYMTRKPYSQKKIKLNFENPQLLGGWD |
| | | ANKEKDYATIILRRNGLYYLAIMDKDSRKLLGKAMPSDGECYEKMVYKF |
| | | FKDVTTMIPKCSTQLKDVQAYFKVNTDDYVLNSKAFNKPLTITKEVEDL |
| | | NNVLYGKYKKFQKGYLTATGDNVGYTHAVNVWIKFCMDFLNSYDSTCIY |
| | | DESSLKPESYLSLDAFYQDANLLLYKLSFARASVSYINQLVEEGKMYLF |
| | | QIYNKDFSEYSKGTPNMHTLYWKALFDERNLADVVYKLNGQAEMFYRKK |
| | | SIENTHPTHPANHPILNKNKDNKKKESLFDYDLIKDRRYTVDKEMFHVP |
| | | ITMNFKSVGSENINQDVKAYLRHADDMHIIGIDRGERHLLYLVVIDLQG |
| | | NIKEQYSLNEIVNEYNGNTYHTNYHDLLDVREEERLKARQSWQTIENIK |
| | | ELKEGYLSQVIHKITQLMVRYHAIVVLEDLSKGFMRSRQKVEKQVYQKF |
| | | EKMLIDKLNYLVDKKTDVSTPGGLLNAYQLTCKSDSSQKLGKQSGFLFY |
| | | IPAWNTSKIDPVTGFVNLLDTHSLNSKEKIKAFFSKFDAIRYNKDKKWF |
| | | EFNLDYDKFGKKAEDTRTKWTLCTRGMRIDTERNKEKNSQWDNQEVDLT |
| | | TEMKSLLEHYYIDIHGNLKDAISAQTDKAFFTGLLHILKLTLQMRNSIT |
| | | GTETDYLVSPVADENGIFYDSRSCGNQLPENADANGAYNIARKGLMLIE |
| | | QIKNAEDLNNVKFDISNKAWLNFAQQKPYKNG |
| ART14 | 14 | MAKNFEDFKRLYSLSKTLRFEAKPIGATLDNIVKSDLLDEDEHRAASYV |
| | | KVKKLIDEYHKVFIDRVLDDGCLPLENKGNNNSLAEYYESYVSRAQDED |
| | | AKKKFKEIQQNLRSVIAKKLTEDKAYANLFGNKLIESYKDKEDKKKIID |
| | | SDLIQFINTAESTQLDSMSQDEAKELVKEFWGFVTYFYGFFDNRKNMYT |
| | | AEEKSTGIAYRLVNENLPKFIDNIEAFNRAITRPEIQENMGVLYSDFSE |
| | | YLNVESIQEMFQLDYYNMLLTQKQIDVYNAIIGGKTDDEHDVKIKGIND |
| | | YINLYNQKHKDDKLPKLKALFKQILSDRNAISWLPEEFNSDQEVLNAIK |
| | | DCYERLSENVLGDKVLKSMLGSLADYSLDGIFIRNDLQLTDISQKMEGN |
| | | WSVIQNAIMQNIKHVAPARKHKESEEEYENRIAGIFKKADSFSISYIDA |
| | | CLNETDPNNAYFVENYFATLGAVDTPTMQRENLFALVQNAYTEITALLH |
| | | SDYPTEKNLAQDKANVAKIKALLDAIKSLQHFVKPLLGKGDESDKDERF |
| | | YGELASLWAELDTMTPLYNMIRNYMTRKPYSQKKIKLNFENPQLLGGWD |
| | | ANKEKDYATIILRRNGLYYLAIMNKDSKKLLGKAMPSDGECYEKMVYKL |
| | | LPGANKMLPKVFFAKSRMEDFKPSKELVEKYYNGTHKKGKNFNIQDCHN |
| | | LIDYFKQSIDKHEDWSKFGFKFSDTSTYEDLSGFYREVEQQGYKLSFAR |
| | | VSVSYINQLVEEGKMYLFQIYNKDFSEYSKGTPNMHTLYWKALFDERNL |
| | | ADVVYKLNGQAEMFYRKKSIENTHPTHPANHPILNKNKDNKKKESLFGY |
| | | DLIKDRRYTVDKFLFHVPITMNFKSSGSENINQDVKAYLRHADDMHIIG |
| | | IDRGERHLLYLVVIDLQGNIKEQFSLNEIVNDYNGNTYHTNYHDLLDVR |
| | | EDERLKARQSWQTIENIKELKEGYLSQVIHKITQLMVKYHAIVVLEDLN |
| | | MGFMRGRQKVEKQVYQKFEKMLIEKLNYLVDKKADASVSGGLLNAYQLT |
| | | SKEDSFQKLGKQSGFLFYIPAWNTSKIDPVTGFVNLLDTRYQNVEKAKS |
| | | FFSKFDAIRYNKDKEWFEFNLDYDKFGKKAEGTRTKWTLCTRGMRIDTF |
| | | RNKEKNSQWDNQEVDLTAEMKSLLEHYYIDIHSNLKDAISAQTDKAFFT |
| | | GLLHILKLTLQMRNSITGTETDYLVSPVVDENGIFYDSRSCGDELPENA |
| | | DANGAYNIARKGLMMIEQIKDAKDLDNLKFDISNKAWLNFAQQKPYKNG |
| ART15 | 15 | MLFQDFTHLYPLSKTVRFELKPIGRTLEHIHAKNFLSQDETMADMYQKV |
| | | KVILDDYHRDFIADMMGEVKLTKLAEFYDVYLKFRKNPKDDELQKQLKD |
| | | LQAVLRKESVKPIGNGGKYKAGHDRLFGAKLFKDGKELGDLAKFVIAQE |
| | | GKSSPKLAHLAHFEKFSTYFTGFHDNRKNMYSDEDKHTAIAYRLIHENL |
| | | PRFIDNLQILTTIKQKHSALYDQIINELTASGLDVSLASHLDGYHKLLT |
| | | QEGITAYNRIIGEVNGYTNKHNQICHKSERIAKLRPLHKQILSDGMGVS |
| | | FLPSKFADDSEMCQAVNEFYRHYADVFAKVQSLEDGEDDHQKDGIYVEH |
| | | KNLNELSKQAFGDFALLGRVLDGYYVDVVNPEFNERFAKAKTDNAKAKL |
| | | TKEKDKFIKGVHSLASLEQAIKHHTARHDDESVQAGKLGQYFKHGLAGV |
| | | DNPIQKIHNNHSTIKGFLERERPAGERALPKIKSGKNPEMTQLRQLKEL |
| | | LDNALNVAHFAKLLMTKTTLDNQDGNFYGEFGVLYDELAKIPTLYNKVR |
| | | DYLSQKPFSTEKYKLNFGNPTLLNGWDLNKEKDNFGVILQKDGCYYLAL |
| | | LDKAHKKVFDNAPNTGKNVYQKMIYKLLPGPNKMLPRVFFAKSNLDYYN |
| | | PSAELLDKYAQGTHKKGDNFNLKDCHALIDFFKAGINKHPEWQNFGFKF |
| | | SPTSSYRDLSDFYREVEPQGYQVKFVDINADYIDELVEQGQLYLFQIYN |
| | | KDFSPKAHGKPNLHTLYFRALFSEDNLANPIYKLNGEAQIFYRKASLGM |
| | | NETTIHRAGEILENKNPDNPKERVFTYDIIKDRRYTQDKFMLHVPITMN |
| | | FGVQGMTIKEFNKKVNQSIRQYDDVNVIGIDRGERHLLYLTVINSKGEI |
| | | LEQRSLNDITTASANGTQMTTPYHKILDKREIERLNARVGWGEIETIKE |
| | | LKSGYLSHVVHQVSQLMLKYNAIVVLEDLNFGFKRGRFKVEKQIYQNFE |
| | | NALIKKLNHLELKDKADDEIGSYKNALQLTNNFTDLKNIGKQTGFLFYV |
| | | PAWNTSKIDPETGFVDLLKPRYENIAQSQAFFGKFDKICYNADKDYFEF |
| | | HIDYAKFTDKAKNSRQTWTICSHGDKRYVYDKTANQNKGATKGINVNDE |
| | | LKSLFARYHINEKQPNLVMDICQNNDKEFHKSLMYLLKTLLALRYSNAS |
| | | SDEDFILSPVANDEGVFFNSALADDTQPQNADANGAYHIALKGLWLLNE |
| | | LKNSDDLNKVKLAIDNQTWLNFAQNR |
| ART16 | 16 | MLFQDFTHLYPLSKTVRFELKPIGKTLEHIHAKNFLSQDETMADMYQKV |
| | | KAILDDYHRDFITKMMSEVTLTKLPEFYEVYLALRKNPKDDTLQKQLTE |
| | | IQTALREEVVKPIDSGGKYKAGYERLFGAKLFKDGKELGDLAKFVIAQE |
| | | GESSPKLPQIAHFEKESTYFTGFHDNRKNMYSSDDKHTAIAYRLIHENL |
| | | PRFIDNLQILVTIKQKHSVLYDQIVNELNANGLDVSLASHLDGYHKLLT |
| | | QEGITAYNRIIGEVNSYTNKHNQICHKSERIAKLRPLHKQILSDGMGVS |
| | | FLPSKFADDSEMCQAVNEFYRHYAHVFAKVQSLEDREDDYQKDGIYVEH |
| | | KNLNELSKQAFGDFALLGRVLDGYYVDVVNPEFNDKFAKAKTDNAKEKL |
| | | TKEKDKFIKGVHSLASLEQAIEHYIAGHDDESVQAGKLGQYFKHGLAGV |
| | | DNPIQKIHNSHSTIKGFLERERPAGERTLPKIKSDKSLEMTQLRQLKEL |
| | | LDNALNVVHFAKLLTTKTTLDNQDGNFYGEFGALYDELAKIATLYNKVR |
| | | DYLSQKPFSTEKYKLNFGNPTLLNGWDLNKEKDNFGVILQKDGCYYLAL |
| | | LDKAHKKVFDNAPNTGKSVYQKMVYKLLPGPNKMLPKVFFAKSNLDYYN |
| | | PSAELLDKYAQGTHKKGDNFNLKDCHALIDFFKASINKHPEWQHFGFEF |
| | | SLTSSYQDLSDFYREVEPQGYQVKFVDIDADYIDELVEQGQLYLFQIYN |
| | | KDFSPKAHGKPNLHTLYFKALFSEDNLANPIYKLNGEAEIFYRKASLDM |
| | | NETTIHRAGEVLENKNPDNPKERQFVYDIIKDKRYTQDKFMLHVPITMN |
| | | FGVQGMTIKEFNKKVNQSIQQYDEVNVIGIDRGERHLLYLTVINSKGEI |
| | | LEQRSLNDIITTSANGTQMTTPYHKILDKREIERLNARVGWGEIETIKE |
| | | LKSGYLSHVVHQISQLMLKYNAIVVLEDLNFGFKRGRFKVEKQIYQNFE |
| | | NALIKKLNHLVLKDKADNEIGSYKNALQLTNNFTDLKSIGKQTGFLFYV |
| | | PAWNTSKIDPVTGFVDLLKPRYENIAQSQAFFDKEDKICYNADKGYFEF |
| | | HIDYAKFTDKAKNSRQIWTICSHGDKRYVYDKTANQNKGATIGINVNDE |
| | | LKSLFARYRINDKQPNLVMDICQNNDKEFHKSLTYLLKALLALRYSNAS |
| | | SDEDFILSPVANDKGVFFNSALADDTQPQNADANGAYHIALKGLWLLNE |
| | | LKNSDDLDKVKLAIDNQTWLNFAQNR |
| ART17 | 17 | MLFQDFTHLYPLSKTVRFELKPIGKTLEHIHAKNFLSQDETMADMYQKV |
| | | KAILDDYHRDFITKMMSEVTLTKLPEFYEVYLALRKNPKDDTLQKQLTE |
| | | IQTALREEVVKPIDSGGKYKAGYERLFGAKLFKDGKELGDLAKFVIAQE |
| | | GESSPKLPQIAHFEKFSTYFTGFHDNRKNMYSSDDKHTAIAYRLIHENL |
| | | PRFIDNLQILVTIKQKHSVLYDQIVNELNANGLDVSLASHLDGYHKLLT |
| | | QEGITAYNRIIGEVNSYTNKHNQICHKSERIAKLRPLHKQILSDGMGVS |
| | | FLPSKFADDSEMCQAVNEFYRHYAHVFAKVQSLEDREDDYQKDGIYVEH |
| | | KNLNELSKQAFGDFALLGRVLDGYYVDVVNPEFNDKFAKAKTDNAKEKL |
| | | TKEKDKFIKGVHSLASLEQAIEHYIAGHDDESVQAGKLGQYFKHGLAGV |
| | | DNPIQKIHNSHSTIKGFLERERPAGERTLPKIKSDKSLEMTQLRQLKEL |
| | | LDNALNVVHFAKLLTTKTTLDNQDGNFYGEFGALYDELAKIATLYNKVR |
| | | DYLSQKPFSTEKYKLNFGNPTLLNGWDLNKEKDNFGVILQKDGCYYLAL |
| | | LDKAHKKVFDNAPNTGKSVYQKMVYKLLPGSNKMLPKVFFAKSNLDYYN |
| | | PSAELLDKYAQGTHKKGDNFNLKDCHALIDFFKASINKHPEWQHFGFEF |
| | | SLTSSYQDLSDFYREVEPQGYQVKFVDIDADYIDELVEQGQLYLFQIYN |
| | | KDFSPKAHGKPNLHTLYFKALFSEDNLANPIYKLNGEAEIFYRKASLDM |
| | | NETTIHRAGEVLENKNPDNPKERQFVYDIIKDKRYTQDKEMLHVPITMN |
| | | FGVQGMTIKEFNKKVNQSIQQYDEVNVIGIDRGERHLLYLTVINSKGEI |
| | | LEQRSLNDIITTSANGTQMTTPYHKILDKREIERLNARVGWGEIETIKE |
| | | LKSGYLSHVVHQISQLMLKYNAIVVLEDLNFGFKRGRFKVEKQIYQNFE |
| | | NALIKKLNHLVLKDKADNEIGSYKNALQLTNNFTDLKSIGKQTGFLFYV |
| | | PAWNTSKIDPVTGFVDLLKPRYENIAQSQAFFDKFDKICYNADKGYFEF |
| | | HIDYAKFTDKAKNSRQIWTICSHGDKRYVYDKTANQNKGATIGINVNDE |
| | | LKSLFARYRINDKQPNLVMDICQNNDKEFHKSLTYLLKALLALRYSNAS |
| | | SDEDFILSPVANDKGVFFNSALADDTQPQNADANGAYHIALKGLWLLNE |
| | | LKNSDDLDKVKLAIDNQTWLNFAQNR |
| ART18 | 18 | MKYTDFTGIYPVSKTLRFELIPQGSTVENMKREGILNNDMHRADSYKEM |
| | | KKLIDEYHKVFIERCLSDESLKYDDTGKHDSLEEYFFYYEQKRNDKTKK |
| | | IFEDIQVALRKQISKRFTGDTAFKRLFKKELIKEDLPSFVKNDPVKTEL |
| | | IKEFSDFTTYFQEFHKNRKNMYTSDAKSTAIAYRIINENLPKFIDNINA |
| | | FHIVAKVPEMQEHFKTIADELRSHLQVGDDIDKMENLQFFNKVLTQSQL |
| | | AVYNAVIGGKSEGNKKIQGINEYVNLYNQQHKKARLPMLKLLYKQILSD |
| | | RVAISWLQDEFDNDQDMLDTIEAFYNKLDSNETGVLGEGKLKQILMGLD |
| | | GYNLDGVFLRNDLQLSEVSQRLCGGWNIIKDAMISDLKRSVQKKKKETG |
| | | ADFEERVSKLFSAQNSFSIAYINQCLGQAGIRCKIQDYFACLGAKEGEN |
| | | EAETTPDIFDQIAEAYHGAAPILNARPSSHNLAQDIEKVKAIKALLDAL |
| | | KRLQRFVKPLLGRGDEGDKDSFFYGDEMPIWEVLDQLTPLYNKVRNRMT |
| | | RKPYSQEKIKLNFENSTLLNGWDLNKEHDNTSVILRREGLYYLGIMNKN |
| | | YNKIFDANNVETIGDCYEKMIYKLLPGPNKMLPKVFFSKSRVQEFSPSK |
| | | KILEIWESKSFKKGDNFNLDDCHALIDFYKDSIAKHPDWNKENFKFSDT |
| | | QSYTNISDFYRDVNQQGYSLSFTKVSVDYVNRMVDEGKLYLFQIYNKDF |
| | | SPQSKGTPNMHTLYWRMLEDERNLHNVIYKLNGEAEVFYRKASLRCDRP |
| | | THPAHQPITCKNENDSKRVCVFDYDIIKNRRYTVDKEMFHVPITINYKC |
| | | TGSDNINQQVCDYLRSAGDDTHIIGIDRGERNLLYLVIIDQHGTIKEQF |
| | | SLNEIVNEYKGNTYCTNYHTLLEEKEAGNKKARQDWQTIESIKELKEGY |
| | | LSQVIHKISMLMQRYHAIVVLEDLNGSFMRSRQKVEKQVYQKFEHMLIN |
| | | KLNYLVNKQYDAAEPGGLLHALQLTSRMDSFKKLGKQSGELFYIPAWNT |
| | | SKIDPVTGFVNLFDTRYCNEAKAKEFFEKFDDISYNDERDWFEFSFDYR |
| | | HFTNKPTGTRTQWTLCTQGTRVRTFRNPEKSNHWDNEEFDLTQAFKDLF |
| | | NKYGIDIASGLKARIVNGQLTKETSAVKDFYESLLKLLKLTLQMRNSVT |
| | | GTDIDYLVSPVADKDGIFFDSRTCGSLLPANADANGAFNIARKGLMLLR |
| | | QIQQSSIDAEKIQLAPIKNEDWLEFAQEKPYL |
| ART19 | 19 | METFSGFTNLYPLSKTLRFRLIPVGETLKYFIGSGILEEDQHRAESYVK |
| | | VKAIIDDYHRAYIENSLSGFELPLESTGKENSLEEYYLYHNIRNKTEEI |
| | | QNLSSKVRTNLRKQVVAQLTKNEIFKRIDKKELIQSDLIDFVKNEPDAN |
| | | EKIALISEFRNFTVYFKGFHENRRNMYSDEEKSTSIAFRLIHENLPKFI |
| | | DNMEVFAKIQNTSISENFDAIQKELCPELVTLCEMEKLGYENKTLSQKQ |
| | | IDAYNTVIGGKTTSEGKKIKGLNEYINLYNQQHKQEKLPKMKLLFKQIL |
| | | SDRESASWLPEKFENDSQVVGAIVNEWNTIHDTVLAEGGLKTIIASLGS |
| | | YGLEGIFLKNDLQLTDISQKATGSWGKISSEIKQKIEVMNPQKKKESYE |
| | | TYQERIDKIFKSYKSFSLAFINECLRGEYKIEDYFLKLGAVNSSSLQKE |
| | | NHFSHILNTYTDVKEVIGFYSESTDTKLIRDNGSIQKIKLFLDAVKDLQ |
| | | AYVKPLLGNGDETGKDERFYGDLIEYWSLLDLITPLYNMVRNYVTQKPY |
| | | SVDKIKINFQNPTLLNGWDLNKETDNTSVILRRDGKYYLAIMNNKSRKV |
| | | FLKYPSGTDRNCYEKMEYKLLPGANKMLPKVFFSKSRINEFMPNERLLS |
| | | NYEKGTHKKSGTCFSLDDCHTLIDFFKKSLDKHEDWKNFGFKESDTSTY |
| | | EDMSGFYKEVENQGYKLSFKPIDATYVDQLVDEGKIFLFQIYNKDFSEH |
| | | SKGTPNMHTLYWKMLFDETNLGDVVYKLNGEAEVFFRKASINVSHPTHP |
| | | ANIPIKKKNLKHKDEERILKYDLIKDKRYTVDQFQFHVPITMNFKADGN |
| | | GNINQKAIDYLRSASDTHIIGIDRGERNLLYLVVIDGNGKICEQFSLNE |
| | | IEVEYNGEKYSTNYHDLLNVKENERKQARQSWQSIANIKDLKEGYLSQV |
| | | IHKISELMVKYNAIVVLEDLNAGFMRGRQKVEKQVYQKFEKKLIEKLNY |
| | | LVFKKQSSDLPGGLMHAYQLANKFESFNTLGKQSGFLFYIPAWNTSKMD |
| | | PVTGFVNLFDVKYESVDKAKSFFSKEDSIRYNVERDMFEWKENYGEFTK |
| | | KAEGTKTDWTVCSYGNRIITFRNPDKNSQWDNKEINLTENIKLLFERFG |
| | | IDLSSNLKDEIMQRTEKEFFIELISLFKLVLQMRNSWTGTDIDYLVSPV |
| | | CNENGEFFDSRNVDETLPQNADANGAYNIARKGMILLDKIKKSNGEKKL |
| | | ALSITNREWLSFAQGCCKNG |
| ART20 | 20 | METFSGFTNLYPLSKTLRFRLIPVGETLKHFIDSGILEEDQHRAESYVK |
| | | VKAIIDDYHRAYIENSLSGFELPLESTGKENSLEEYYLYHNIRNKTEEI |
| | | QNLSSKVRTNLRKQVVVQLTKNEIFKRIDKKELIQSDLIDFVKNEPDAN |
| | | EKIALISEFRNFTVYFKGFHENRRNMYSDEEKSTSIAFRLIHENLPKFI |
| | | DNMEVFAKIQNTSISENFDAIQKELCPELVTLCEMEKLGYENKTLSQKQ |
| | | IDAYNTVIGGKTTSEGKKIKGLNEYINLYNQQHKQEKLPKMKLLFKQIL |
| | | SDRESASWLPEKFENDSQVVGAMVNEWNTIHDTVLAEGGLKTIIASLGS |
| | | YGLEGIFLKNDLQLTDISQKATGSWSKISSEIKQKIEVMNPQKKKESYE |
| | | SYQERIDKLFKSYKSFSLAFINECLRGEYKIEDYFLKLGAVNSSSLQKE |
| | | NHFSHILNAYTDVKEAIGFYSESTDTKLIQDNDSIQKIKQFLDAVKDLQ |
| | | AYVKPLLGNGDETGKDERFYGDLIEYWSLLDLITPLYNMVRNYVTQKPY |
| | | SVDKIKINFQNPTLLNGWDLNKETDNTSVILRRDGKYYLAIMNNKSRKV |
| | | FLKYPSGTDGNCYEKMEYKLLPGANKMLPKVFFSKSRINEFMPNERLLS |
| | | NYEKGTHKKSGICFSLDDCHTLIDFFKKSLDKHEDWKNFGFKESDTSTY |
| | | EDMSGFYKEVENQGYKLSFKPIDATYVDQLVDEGKIFLFQIYNKDFSEH |
| | | SKGTPNMHTLYWKMLFDETNLGDVVYKLNGEAEVFFRKASINVSHPTHP |
| | | ANIPIKKKNLKHKDEERILKYDLIKDKRYTVDQFQFHVPITMNFKADGN |
| | | GNINQKAIDYLCSASDTHIIGIDRGERNLLYLVVIDGNGKICEQFSLNE |
| | | IEVEYNGEKYSTNYHDLLNVKENERKQARQSWQSIANIKDLKEGYLSQV |
| | | IHKISELMVKYNAIVVLEDLNAGFMRGRQKVEKQVYQKFEKKLIEKLNY |
| | | LVFKKQSSDLPGGLMHAYQLANKFESFNALGKQSGFLFYIPAWNTSKMD |
| | | PVTGFVNLFDVKYESVDKAKSFFSKFDSMRYNVERDMFEWKENYGEFTK |
| | | KAEGTKTDWTVCSYGNRIITFRNPDKNSQWDNKEINLTENIKLLFERFG |
| | | IDLSSNLKDEIMQRTEKEFFIELISLFKLVLQMRNSWTGTDIDYLVSPV |
| | | CNENGEFFDSRNVDETLPQNADANGAYNIARKGMILLDKIKKSNGEKKL |
| | | ALSITNREWLSFAQGCCKNG |
| ART21 | 21 | METFSGFTNLYPLSKTLRFRLIPVGETLKHFIGSGILEEDQHRAESYVK |
| | | VKAIIDDYHRTYIENSLSGFELPLESTGKENSLEEYYLYHNIRNKTEEI |
| | | QNLSSKVRTNLRKQVVTQLTKNEIFKRIDKKELIQSDLIDFVKNEPDAN |
| | | EKIALISEFRNFTVYFKGFHENRRNMYSDEEKSTSIAFRLIHENLPKFI |
| | | DNMEVFAKIQNTSISENFDAIQKELCPELVTLCEMEKLGYENKTLSQKQ |
| | | IDAYNTVIGGKTTSEGKKIKGLNEYINLYNQQHKQEKLPKMKLLFKQIL |
| | | SDRESASWLLEKFENDSQVVGAMVNFWNTIHDTVLAEGGLKTIIASLGS |
| | | YGLEGIFLKNDLQLTDISQKATGSWSKISSEIKQKIEAMNPQKKKESYE |
| | | SYQERIDKLFKSYKSFSLAFVNECLRGEYKIEDYFLKLGAVNSSLLQKE |
| | | NHFSHILNTYTDVKEVIGFYSESTDTKLIQDNDSIQKIKQFLDAVKDLQ |
| | | AYVKPLLGNSDETGKDERFYGDLIEYWSLLDLITPLYNMVRNYVTQKPY |
| | | SVDKIKINFQNPTLLNGWDLNKEMDNTSVILRRDGKYYLAIMNNKSRKV |
| | | FLKYPSGTDRNCYEKMEYKLLPGANKMLPKVFFSKSRINEFMPNERLLS |
| | | NYEKGTHKKSGTCFSLDDCHTLIDFFKKSLNKHEDWKNFGFKFSDTSTY |
| | | EDMSGFYKEVENQGYKLSFKPIDATYVDQLVDEGKIFLFQIYNKDFSEH |
| | | SKGTPNMHTLYWKMLFDETNLGDVVYKLNGEAEVFFRKASINVSHPTHP |
| | | ANIPIKKKNLKHKDEERILKYDLIKDKRYTVDQFQFHVPITMNFKANGN |
| | | GNINQKAIDYLRSASDTHIIGIDRGERNLLYLVVIDGNGKICEQFSLNE |
| | | IEVEYNGEKYSTNYHDLLNVKENERKQARQSWQSIANIKDLKEGYLSQV |
| | | IHKISELMVKYNAIVVLEDLNAGFMRGRQKVEKQVYQKFEKKLIEKLNY |
| | | LVFKKQSSDLPGGLMHAYQLANKFESFNTLGKQSGFLFYIPAWNTSKMD |
| | | PVTGFVNLEDVKYESVDKAKSFFSKEDSIRYNVERDMFEWKENYDEFTK |
| | | KAEGTKTDWTVCSYGNRIITFRNPDKNSQWDNKEINLTENIKLLFERFG |
| | | IDLSSNLKDEIMERTEKEFFIELISLFKLVLQMRNSWTGTDIDYLVSPV |
| | | CNENGEFFDSRNVDETLPQNADANGAYNIARKGMILLDKIKKNNGEKKL |
| | | TLSITNREWLSFAQGCCKNG |
| ART22 | 22 | MLFQDFTHLYPLSKTVRFELKPIGKTLEHIHAKNFLSQDKTMADMYQKV |
| | | KAILDDYHRDFIADMMGEVKLTKLAEFCDVYLKERKNPKDDGLQKQLKD |
| | | LQAVLRKEIVKPIGNGGKYKVGYDRLFGAKLFKDGKELGDLAKEVIAQE |
| | | SESSPKLPQIAHFEKFSTYFTGFHDNRKNMYSSDDKHTAIAYRLIHENL |
| | | PRFIDNLQILATIKQKHSALYDQIASELTASGLDVSLASHLGGYHKLLT |
| | | QEGITAYNRIIGEVNSYTNKHNQICHKSERIAKLRPLHKQILSDGMGVS |
| | | FLPSKFADDSEMCQAVNEFYRHYADVFAKVQSLEDREDDYQKDGIYVEH |
| | | KNLNELSKRAFGDFGFLKRFLEEYYADVIDPEFNEKFAKTEPDSDEQKK |
| | | LAGEKDKFVKGVHSLASLEQVIEYYTAGYDDESVQADKLGQYFKHRLAG |
| | | VDNPIQKIHNSHSTIKGFLERERPAGERALPKIKSDKSPEMTQLRQLKE |
| | | LLDNALNVVHFAKLVSTETVLDTRSDKFYGEFRPLYVELAKITTLYNKV |
| | | RDYLSQKPFSTEKYKLNFGNPTLLNGWDLNKEKDNFGVILQKDGCYYLA |
| | | LLDKAHKKVFDNAPNTGKSVYQKMVYKQIANARRDLACLLIINGKVVRK |
| | | TKGLDDLREKYLPYDIYKIYQSESYKVLSPNFNHQDLVKYIDYNKILAS |
| | | GYFEYFDFRFKESSEYKSYKEFLDDVDNCGYKISFCNINADYIDELVEQ |
| | | GQLYLFQIYNKDFSPKAHGKPNLHTLYFKALFSEDNLANPIYKLNGEAQ |
| | | IFYRKASLDMNETTIHRAGEVLENKNPDNPKQRQFVYDIIKDKRYTQDK |
| | | FMLHVPITMNFGVQGMTIEGENKKVNQSIQQYDDVNVIGIDRGERHLLY |
| | | LTVINSKGEILEQRSLNDIITTSANGTQMTTPYHKILNKKKEGRLQARK |
| | | DWGEIETIKELKAGYLSHVVHQISQLMLKYNAIVVLEDLNFGFKRGRLK |
| | | VENQVYQNFENALIKKLNHLVLKDKTDDEIGSYKNALQLTNNFTDLKSI |
| | | GKQTGFLFYVPARNTSKIDPETGFVDLLKPRYENITQSQAFFGKEDKIC |
| | | YNTDKGYFEFHIDYAKFTDEAKNSRQTWVICSHGDKRYVYNKTANQNKG |
| | | ATKGINVNDELKSLFACHHINDKQPNLVMDICQNNDKEFHKSLMYLLKA |
| | | LLALRYSNANSDEDFILSPVANDEGVFFNSALADDTQPQNADANGAYHI |
| | | ALKGLWVLEQIKNSDDLDKVDLEIKDDEWRNFAQNR |
| ART23 | 23 | MGKNQNFQEFIGVSPLQKTLRNELIPTETTKKNITQLDLLTEDEIRAQN |
| | | REKLKEMMDDYYRDVIDSTLHAGIAVDWSYLFSCMRNHLRENSKESKRE |
| | | LERTQDSIRSQIYNKFAERADFKDMFGASIITKLLPTYIKQNPEYSERY |
| | | DESMEILKLYGKFTTSLTDYFETRKNIFSKEKISSAVGYRIVEENAEIF |
| | | LQNQNAYDRICKIAGLDLHGLDNEITAYVDGKTLKEVCSDEGFAKAITQ |
| | | EGIDRYNEAIGAVNQYMNLLCQKNKALKPGQFKMKRLHKQILCKGTTSF |
| | | DIPKKFENDKQVYDAVNSFTEIVMKNNDLKRLLNITQNVNDYDMNKIYV |
| | | AADAYSTISQFISKKWNLIEECLLDYYSDNLPGKGNAKENKVKKAVKEE |
| | | TYRSVSQLNELIEKYYVEKTGQSVWKVESYISRLAETITLELCHEIEND |
| | | EKHNLIEDDDKISKIKELLDMYMDAFHIIKVERVNEVLNFDETFYSEMD |
| | | EIYQDMQEIVPLYNHVRNYVTQKPYKQEKYRLYENTPTLANGWSKNKEY |
| | | DNNAIILMRDDKYYLGILNAKKKPSKQTMAGKEDCLEHAYAKMNYYLLP |
| | | GANKMLPKVFLSKKGIQDYHPSSYIVEGYNEKKHIKGSKNFDIRFCRDL |
| | | IDYFKECIKKHPDWNKENFEFSATETYEDISVFYREVEKQGYRVEWTYI |
| | | NSEDIQKLEEDGQLFLFQIYNKDFAVGSTGKPNLHTLYLKNLFSEENLR |
| | | DIVLKLNGEAEIFFRKSSVQKPVIHKCGSILVNRTYEITESGTTRVQSI |
| | | PESEYMELYRYENSEKQIELSDEAKKYLDKVQCNKAKTDIVKDYRYTMD |
| | | KFFIHLPITINFKVDKGNNVNAIAQQYIAEQEDLHVIGIDRGERNLIYV |
| | | SVIDMYGRILEQKSFNLVEQVSSQGTKRYYDYKEKLQNREEERDKARKS |
| | | WKTIGKIKELKEGYLSSVIHEIAQMVVKYNAIIAMEDLNYGFKRGRFKV |
| | | ERQVYQKFETMLISKLNYLADKSQAVDEPGGILRGYQMTYVPDNIKNVG |
| | | RQCGIIFYVPAAYTSKIDPTTGFINAFKRDVVSTNDAKENFLMKEDSIQ |
| | | YDIEKGLFKFSFDYKNFATHKLTLAKTKWDVYINGTRIQNMKVEGHWLS |
| | | MEVELTTKMKELLDDSHIPYEEGQNILDDLREMKDITTIVNGILEIFWL |
| | | TVQLRNSRIDNPDYDRIISPVLNNDGEFFDSDEYNSYIDAQKAPLPIDA |
| | | DANGAFCIALKGMYTANQIKENWVEGEKLPADCLKIEHASWLAFMQGER |
| | | G |
| ART24 | 24 | MNTSLFSSFTRQYPVTKTLRFELKPMGATLGHIQQKGFLHKDEELAKIY |
| | | KKIKELLDEYHRAFIADTLGDAQLVGLDDFYADYQALKQDSKNSHLKDK |
| | | LTKTQDNLRKQITKNFEKTPQLKERYKRLFTKELFKAGKDKGDLEKWLI |
| | | NHDSEPNKAEKISWIHQFENFTTYFQGFYENRKNMYSDEVKHTAIAYRL |
| | | IHENLPRFVDNIQVLSKIKSDYPDLYHELNHLDSRTIDFADEKEDDMLQ |
| | | MDFYHHLLIQSGITAYNTLLGGKVLEGGKKLQGINELINLYGQKHKIKI |
| | | AKLKPLHKQILSDGQSVSFLPKKFDNDYELCQTVNHFYREYVAIFDELV |
| | | VLFQKFYDYDKDNIYINHQQLNQLSHELFADERLLSRALDFYYCQIIDG |
| | | DENNKINNAKSQNAKEKLLKEKERYTKSNHSINELQKAINHYASHHEDT |
| | | EVKVISDYFSATNIRNMIDGIHHHESTIKGFLEKDNNQGESYLPKQKNS |
| | | NDVKNLKLFLDGVLRLIHFIKPLALKSDDTLEKEEHFYGEFMPLYDKLV |
| | | MFTLLYNKVRDYISQKPYNDEKIKLNFGNSTLLNGWDVNKEKDNFGVIL |
| | | CKEGLYYLAILDKSHKKVEDNAPKATSSHTYQKMVYKLLPGPNKMLPKV |
| | | FFAKSNIGYYQPSAQLLENYEKGTHKKGSNFSLTDCHHLIDFFKSSIAK |
| | | HPEWKEFGFRFSDTHTYQDLSDFYKEIEPQSYKVKFIDIDADYIDDLVE |
| | | KGQLYLFQLYNKDFSKQSYGKPNLHTLYFKSLFSDDNLKNPIYKLNGEA |
| | | EIFYRRASLSVSDTTIHQAGEILTPKNPNNTHNRTLSYDVIKNKRYTTD |
| | | KFFLHIPITMNFGIENTGFKAFNHQVNTTLKNADKKDVHIIGIDRGERH |
| | | LLYVSVIDGDGRIVEQRTLNDIVSISNNGMSMSTPYHQILDNREKERLA |
| | | ARTDWGDIKNIKELKAGYLSHVVHEVVQMMLKYNAMIVLEDLNFGFKHG |
| | | RFKVEKQVYQNFENALIKKLNYLVLKNADNHQLGSVRKALQLTNNETDI |
| | | KSIGKQTGFIFYVPAWNTSKIDPTTGFVDLLKPRYENMAQAQSFISREK |
| | | KIAYNHQLDYFEFEFDYADFYQKTIDKKRIWTLCTYGDVRYYYDHKTKE |
| | | TKTVNITKELKSLLDKHDLSYQNGHNLVDELANSHDKSLLSGVMYLLKV |
| | | LLALRYSHAQKNEDFILSPVMNKDGVFFDSRFADDVLPNNADANGAYHI |
| | | ALKGLWVLNQIQSADNMDKIDLSISNEQWLHFTQSR |
| ART25 | 25 | MVGNKISNSFDSFTGINALSKTLRNELIPSDYTKRHIAESDFIAADTNK |
| | | NEDQYVAKEMMDDYYRDFISKVLDNLHDIEWKNLFELMHKAKIDKSDAT |
| | | SKELIKIQDMLRKKIGKKESQDPEYKVMLSAGMITKILPKYILEKYETD |
| | | REDRLEAIKRFYGFTVYFKEFWASRQNVESDKAIASSISYRIIHENAKI |
| | | YMDNLDAYNRIKQIACEEIEKIEEEAYDFLQGDQLDVVYTEEAYGRFIS |
| | | QSGIDLYNNICGVINAHMNLYCQSKKCSRSKFKMQKLHKQILCKAETGF |
| | | EIPLGFQDDAQVINAINSFNALIKEKNIISRLRTIGKSISLYDVNKIYI |
| | | SSKAFENVSVYIDHKWDVIASSLYKYFSEIVKGNKDNREEKIQKEIKKV |
| | | KSCSLGDLQRLVNSYYKIDSTCLEHEVTEFVTKIIDEIDNFQITDEKEN |
| | | DKISLIQNEQIVMDIKTYLDKYMSIYHWMKSFVIDELVDKDMEFYSELD |
| | | ELNEDMSEIVNLYNKVRNYVTQKPYSQEKIKLNFGSPTLADGWSKSKEF |
| | | DNNAIILIRDEKIYLAIFNPRNKPAKTVISGHDVCNSETDYKKMNYYLL |
| | | PGASKTLPHVFIKSRLWNESHGIPDEILRGYELGKHLKSSVNFDVEFCW |
| | | KLIDYYKECISCYPNYKAYNFKFADTESYNDISEFYREVECQGYKIDWT |
| | | YISSEDVEQLDRDGQIYLFQIYNKDFAPNSKGMDNLHTKYLKNIFSEDN |
| | | LKNIVIKLNGEAELFYRKSSVKKKVEHKKGTILVNKTYKVEDNTENSKE |
| | | KRVIIESVPDDCYMELVDYWRNGGIGILSDKAVQYKDKVSHYEATMDIV |
| | | KDRRYTVDKFFIHLPITINFKADGRININEKVLKYIAENDELHVIGIDR |
| | | GERNLLYVSVINKKGKIVEQKSFNMIESYETVTNIVRRYNYKDKLVNKE |
| | | SARTDARKNWKEIGKIKEIKEGYLSQVIHEISKMVLKYNAIIVMEDLNY |
| | | GFKRGRFRVERQVYQKFENMLISKLAYLVDKSRKADEPGGVLRGYQLTY |
| | | IPDSLEKLGSQCGIIFYVPAAYTSKIDPLTGFVNVENFREYSNFETKLD |
| | | FVRSLDSIRYDTEKKLFSISFDYDNFKTHNTTLAKTKWVIYLRGERIKK |
| | | EHTSYGWKDDVWNVESRIKDLFDSSHMKYDDGHNLIEDILELESSVQKK |
| | | LINELIEIIRLTVQLRNSKSERYDRTEAEYDRIVSPVMDENGRFYDSEN |
| | | YIFNEETELPKDADANGAYCIALKGLYNVIAIKNNWKEGEKFNRKLLSL |
| | | NNYNWEDFIQNRRF |
| ART26 | 26 | MVGNKISNSFDSFTGINALSKTLRNELIPSDYTKRHIAESDFIAADINK |
| | | NEDQYVAKEMMDDYYRDFISKVLDNLHDIEWKNLFELMHKAKIDKSDAT |
| | | SKELIKIQDMLRKKIGKKFSQDPEYKVMLSAGMITKILPKYILEKYETD |
| | | REDRLEAIKRFYGFTVYFKEFWASRQNVESDKAIASSISYRIIHENAKI |
| | | YMDNLDAYNRIKQIACEEIEKIEEEAYDFLQGDQLDVVYTEEAYGRFIS |
| | | QSGIDLYNNICGVINAHMNLYCQSKKCSRSKFKMQKLHKQILCKAETGF |
| | | EIPLGFQDDAQVINAINSENALIKEKNIISRLRTIGKSISLYDVNKIYI |
| | | SSKAFENVSVYIDHKWDVIASSLYKYFSEIVKGNKDNREEKIQKEIKKV |
| | | KSCSLGDLQRLVNSYYKIDSTCLEHEVTEFVTKIIDEIDNFQITDEKEN |
| | | DKISLIQNEQIVMDIKTYLDKYMSIYHWMKSFVIDELVDKDMEFYSELD |
| | | ELNEDMSEIVNLYNKVRNYVTQKPYSQEKIKLNFGSPTLADGWSKSKEF |
| | | DNNAIILIRDEKIYLAIFNPRNKPAKTVISGHDVCNSETDYKKMNYYLL |
| | | PGASKTLPHVFIKSRLWNESHGIPDEILRGYELGKHLKSSVNEDVEFCW |
| | | KLIDYYKECISCYPNYKAYNFKFADTESYNDISEFYREVECQGYKIDWT |
| | | YISSEDVEQLDRDGQIYLFQIYNKDFAPNSKGMDNLHTKYLKNIFSEDN |
| | | LKNIVIKLNGEAELFYRKSSVKKKVEHKKGTILVNKTYKVEDNTENSKE |
| | | KRVIIESVPDDCYMELVDYWRNGGIGILSDKAVQYKDKVSHYEATMDIV |
| | | KDRRYTVDKFFIHLPITINFKADGRININEKVLKYIAENDELHVIGIDR |
| | | GERNLLYVSVINKKGKIVEQKSENMIESYETVTNIVRRYNYKDKLVNKE |
| | | SARTDARKNWKEIGKIKEIKEGYLSQVIHEISKMVLKYNAIIVMEDLNY |
| | | GFKRGRFRVERQVYQKFENMLISKLAYLVDKSRKADEPGGVLRGYQLTY |
| | | IPDSLEKLGSQCGIIFYVPAAYTSKIDPLTGFVNVENFREYSNFETKLD |
| | | FVRSLDSIRYDTEKKLFSISFDYDNFKTHNTTLAKTKWVIYLRGERIKK |
| | | EHTSYGWKDDVWNVESRIKDLFDSSHMKYDDGHNLIEDILELESSVQKK |
| | | LINELIEIIRLTVQLRNSKSERYDRTEAEYDRIVSPVMDENGRFYDSEN |
| | | YIFNEETELPKDADANGAYCIALKGLYNVIAIKNNWKEGEKFNRKLLSL |
| | | NNYNWFDFIQNRRFQIYLFQIYNKDFAPNSKGMDNLHTKYLKNIFSEDN |
| | | LKNIVIKLNGEAELFYRKSSVKKKVEHKKGTILVNKTYKVEDNTENSKE |
| | | KRVIIESVPDDCYMELVDYWRNGGIGILSDKAVQYKDKVSHYEATMDIV |
| | | KDRRYTVDKFFIHLPITINFKADGRININEKVLKYIAENDELHVIGIDR |
| | | GERNLLYVSVINKKGKIVEQKSENMIESYETVTNIVRRYNYKDKLVNKE |
| | | SARTDARKNWKEIGKIKEIKEGYLSQVIHEISKMVLKYNAIIVMEDLNY |
| | | GFKRGRFRVERQVYQKFENMLISKLAYLVDKSRKADEPGGVLRGYQLTY |
| | | IPDSLEKLGSQCGIIFYVPAAYTSKIDPLTGFVNVENFREYSNFETKLD |
| | | FVRSLDSIRYDTEKRLFSISFDYDNFKTHNTTLAKTKWVIYLRGERIKK |
| | | EHTSYGWKDDVWNVESRIKDLFDSSHMKYDDGHNLIEDILELESSVQKK |
| | | LINELIEIIRLTVQLRNSKSERYDRTEAEYDRIVSPVMDEKGRFYDSEN |
| | | YIFNEETELPKDADANGAYCIALKGLYNVIAIKNNWKEGEKENRKLLSL |
| | | NNYNWFDFIQNRRF |
| ART27 | 27 | MQEHKKISHLTHRNSVQKTIRMQLNPVGKTMDYFQAKQILENDEKLKED |
| | | YQKIKEIADRFYRNLNEDVLSKTGLDKLKDYAEIYYHCNTDADRKRLDE |
| | | CASELRKEIVKNFKNRDEYNKLENKKMIEIVLPQHLKNEDEKEVVASFK |
| | | NFTTYFTGFFTNRKNMYSDGEESTAIAYRCINENLPKHLDNVKAFEKAI |
| | | SKLSKNAVDDLDTTYSGLCGTNLYDVFTVDYFNFLLPQSGITEYNKIIG |
| | | GYTTSDGTKVKGINEYINLYNQQVSKRYKIPNLKILYKQILSESEKVSF |
| | | IPPKFEDDNELLSAVSEFYANDETFDGMPLKKAIDETKLLFGNLDNSSL |
| | | NGIYIQNDRSVINLSNSMFGSWSVIEDLWNKNYDSVNSNSRIKDIQKRE |
| | | DKRKKAYKAEKKLSLSFLQVLISNSENDEIREKSIVDYYKTSLMQLTDN |
| | | LSDKYKEAAPLFNESYANEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDRNKEKDCGAVWLCKDEKYYLAIIDKSNNSILENIDF |
| | | QDCDESDCYEKIIYKLLPGPNKMLPKVFFSEKCKKLLSPSDEILKIRKN |
| | | GTFKKGDKFSLDDCHKLIDFYKESFKKYPNWLIYNFKFKKTNEYNDISE |
| | | FYNDVASQGYNISKMKIPTSFIDKLVDEGKIYLFQLYNKDFSPHSKGTP |
| | | NLHTLYFKMLFDERNLEDVVYKLNGEAEMFYRPASIKYDKPTHPKNTPI |
| | | KNKNTLNDKRASTFPYDLIKDKRYTKWQFSLHFPITMNFKAPDRAMIND |
| | | DVRNLLKSCNNNFIIGIDRGERNLLYVSIIDSNGAIIYQHSLNIIGNKF |
| | | KGKTYETNYREKLETREKERTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEEGGLLHAYQLTNKLESFDKLGMQSGFIFYVRPDFTSKIDPVTGF |
| | | VNLLYPRYENIDKAKDMISREDDIRYNAGEDFFEFDIDYDKFPKTASDY |
| | | RKKWTICTNGERIEAFRNPASNNEWSYRTIILAEKFKELFDNNSINYRD |
| | | SDNLKAEILSQTKGKFFEDFFKLLRLTLQMRNSNPETGEDRILSPVKDK |
| | | NGNFYDSSKYDEKSNLPCDADANGAYNIARKGLWIVEQFKKSDNVSTVE |
| | | PVIHNDKWLKFVQENDMANN |
| ART28 | 28 | MKNLANFTNLYSLQKTLRFELKPIGKTLDWIIKKDLLKQDEILAEDYKI |
| | | VKKIIDRYHKDFIDLAFESAYLQKKSSDSFTAIMEASIQSYSELYFIKE |
| | | KSDRDKKAMEEISGIMRKEIVECFTGKYSEVVKKKFGNLFKKELIKEDL |
| | | LNFCEPDELPIIQKFADETTYFTGFHENRENMYSNEEKATAIANRLIRE |
| | | NLPRYLDNLRIIRSIQGRYKDFGWKDLESNLKRIDKNLQYSDELTENGF |
| | | VYTFSQKGIDRYNLILGGQSVESGEKIQGLNELINLYRQKNQLDRRQLP |
| | | NLKELYKQILSDRTRHSFVPEKFSSDKALLRSLLDFHKEVIQNKNLFEE |
| | | KQVSLLQAIRETLTDLKSFDLDRIYLINDTSLTQISNFVFGDWSKVKTI |
| | | LAIYFDENIANPKDRQRQSNSYLKAKENWLKKNYYSIHELNEAISVYGK |
| | | HSDEELPNTKIEDYFSGLQTKDETKKPIDVLDAIVSKYADLESLLTKEY |
| | | PEDKNLKSDKGSIEKIKNYLDSIKLLQNFLKPLKPKKVQDEKDLGFYND |
| | | LELYLESLESANSLYNKVRNYLTGKEYSDEKIKLNFKNSTLLDGWDENK |
| | | ETSNLSVIFRDINNYYLGILDKQNNRIFESIPEIQSGEETIQKMVYKLL |
| | | PGANNMLPKVFFSEKGLLKFNPSDEITSLYSEGRFKKGDKESINSLHTL |
| | | IDFYKKSLAVHEDWSVENFKFDETSHYEDISQFYRQVESQGYKITFKPI |
| | | SKKYIDTLVEDGKLYLFQIYNKDFSQNKKGGGKPNLHTIYFKSLFEKEN |
| | | LKDVIVKLNGQAEVFFRKKSIHYDENITRYGHHSELLKGRFSYPILKDK |
| | | RFTEDKFQFHFPITLNFKSGEIKQFNARVNSYLKHNKDVKIIGIDRGER |
| | | HLLYLSLIDQDGKILRQESLNLIKNDQNFKAINYQEKLHKKEIERDQAR |
| | | KSWGSIENIKELKEGYLSQVVHTISKLMVEHNAIVVLEDLNFGFKRGRQ |
| | | KVERQVYQKFEKMLIEKLNFLVFKDKEMDEPGGILKAYQLTDNFVSFEK |
| | | MGKQTGFVFYVPAWNTSKIDPKTGFVNFLHLNYENVNQAKELIGKEDQI |
| | | RYNQDRDWFEFQVTTDQFFTKENAPDTRTWIICSTPTKRFYSKRTVNGS |
| | | VSTIEIDVNQKLKELFNDCNYQDGEDLVDRILEKDSKDFFSKLIAYLRI |
| | | LTSLRQNNGEQGFEERDFILSPVVGSDGKFFNSLDASSQEPKDADANGA |
| | | YHIALKGLMNLHVINETDDESLGKPSWKISNKDWLNFVWQRPSLKA |
| ART29 | 29 | MQEHKKISHLTHRNSVQKTIRMQLNPVGKTMDYFQAKQILENDEKLKEN |
| | | YQKIKEIADRFYRNLNEDVLSKTRLDKLKDYTDIYYHCNTDADRKRLDE |
| | | CASELRKEIVKNFKNRDEYNKLENKKMIEIVLPKHLKNEDEKEVVTSFK |
| | | NFTTYFTGFFTNRKNMYSDGEESTAIAYRCINENLPKHLDNVKAFEKAI |
| | | SKLSKNAIDDLDTTYSGLCGTNLYDVFTVDYENFLLPQSGITEYNKIIG |
| | | GYTTNDGTKVKGINEYINLYNQQVSKRDKIPNLKILYKQILSESEKVSF |
| | | IPPKFEDDNELLSAVSEFYANDETFDGMPLKKAIDETKLLFGNLDNPSL |
| | | NGIYIQNDRSVTNLSNSMFGSWSVIEDLWNKNYDSVNSNSRIKDIQKRE |
| | | DKRKKAYKAEKKLSLSFLQVLISNSENDEIREKSIVDYYKTSLMQLTDN |
| | | LSDKYNEAAPLLNENYSNEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDRNKEKDCGAVWLCKDEKYYLAIIDKSNNSILENIDE |
| | | QDCDESDCYEKIIYKLLPGPNKMLPKVFFSEKCKKLLSPSDEILKIYKS |
| | | GTFKTGDKFSLDDCHKLIDFYKESFKKYPNWLIYNFKFKKTNEYNDIRE |
| | | FYNDVALQGYNISKMKIPTSFIDKLVDEGKIYLFQLYNKDESPHSKGTP |
| | | NLHTLYFKMLFDERNLEDVVYRLNGEAEMFYRPASIKYDKPTHPKNTPI |
| | | KNKNTLNDKKTSTFPYDLIKDKRYTKWQFSLHFPITMNFKAPDKAMIND |
| | | DVRNLLKSCNNNFIIGIDRGERNLLYVSVIDSNGAIIYQHSLNIIGNKF |
| | | KEKTYETNYREKLATREKERTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEEGGLLHAYQLTNKLESFDKLGMQSGFIFYVRPDFTSKIDPVTGF |
| | | VNLLYPQYENIDKAKDMISRFDEIRYNAGEDFFEFDIDYDEFPKTASDY |
| | | RKKWTICINGERIEAFRNPANNNEWSYRTIILAEKFKELFDNNSINYRD |
| | | SDDLKAEILSQTKGKFFEDFFKLLRLTLQMRNSNPETGEDRILSPVKDK |
| | | NGNFYDSSKYDEKSKLPCDADANGAYNIARKGLWIVEQFKKADNVSTVE |
| | | PVIHNDQWLKFVQENDMANN |
| ART30 | 30 | MQEHKKISHLTHRNSVQKTIRMQLNPVGKTMDYFQAKQILENDEKLKED |
| | | YQKIKEIADRFYRNLNEDVLSKTGLDKLKDYADIYYHCNTDADRKRLNE |
| | | CASELRKEIVKNFKNRDEYNKLFNKKMIEIVLPKHLKNEDEKEVVASFK |
| | | NFTTYFTGFFTNRKNMYSDGEESTAIAYRCINENLPKHLDNVKVFEKAI |
| | | SKLSKNAIDDLGATYSGLCGTNLYDVFTVDYFNFLLPQSGITEYNKIIG |
| | | GYTTSDGTKVKGINEYINLYNQQVSKRDKIPNLKILYKQILSESEKVSF |
| | | IPPKFEDDNELLSAVSEFYANDETFDGMPLKKAIDETKLLFGNLDNSSL |
| | | NGIYIQNDRSVINLSNSMFGSWSVIEDLWNKNYDSVNSNSRIKDIQKRE |
| | | DKRKKAYKAEKKLSLSFLQVLISNSENDEIREKSIVDYYKTSLMQLTDN |
| | | LSDKYKEAAPLFSENYDNEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDKDKEREYGAVLLCKDEKYYLAIIDKSNNSILENIDF |
| | | QDCNESDYYEKIVYKLLTKINGNLPRVFFSEKRKKLLSPSDEILKIYKS |
| | | GTFKKGDKFSLDDCHKLIDFYKESFKKYPNWLIYNFKFKNTNEYNDISE |
| | | FYNDVASQGYNISKMKIPTTFIDKLVDEGKIYLFQLYNKDFSPHSKGTP |
| | | NLHTLYFKMLFDERNLEDVVYKLNGEAEMFYRPASIKYDKPTHPKNTPI |
| | | KNKNTLNDKKASTFPYDLIKDKRYTKWQFSLHFPITMNFKAPDKAMIND |
| | | DVRNLLKSCNNNFIIGIDRGERNLLYVSVIDSNGAIIYQHSLNIIGNKF |
| | | KGKTYETNYREKLATREKDRTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEEGGLLHAYQLTNKLESFDKLGTQSGFIFYVRPDFTSKIDPVTGF |
| | | VNLLYPRYENIDKAKDMISREDDIRYNAGEDFFEFDIDYDKFPKTASDY |
| | | RKKWTICTNGERIEAFRNPANNNEWSYRTIILAEKFKELFDNNSINYRD |
| | | SDDLKAEILSQTKGKFFEDFFKLLRLTLQMRNSNPETGEDRILSPVKDK |
| | | NGNFYDSSKYDEKSKLPCDADANGAYNIARKGLWIVEQFKKADNVSTVE |
| | | PVIHNDKWLKFVQENDMANN |
| ART31 | 31 | MQERKKISHLTHRNSVKKTIRMQLNPVGKTMDYFQAKQILENDEKLKEN |
| | | YQKIKEIADRFYRNLNEDVLSKTGLDKLKDYAEIYYHCNTDADRKRLNK |
| | | CASELRKEIVKNFKNRDEYNKLFDKRMIEIVLPKHLKNEDEKEVVASFK |
| | | NFTTYFTGFFTNRKNMYSDGEESTAIAYRCINENLPKHLDNVKAFEKAI |
| | | SKLSKNAIDDLDAYSGLCGTNLYDVFTVDYENFLLPQSGITEYNKIIGG |
| | | YTTNDGTKVKGINEYINLYNQQVSKRDKIPNLQILYKQILSESEKVSFI |
| | | PPKFEDDNELLSAVSEFYANDETFDGMPLKKAIDETKLLFGNLDNSSLN |
| | | GIYIQNDRSVINLSNSMFGSWSVIEDLWNKNYDSVNSNSRIKDIQKRED |
| | | KRKKAYKAEKKLSLSFLQVLISNSENDEIRKKSIVDYYKTSLMQLTDNL |
| | | SDKYNEAAPLLNENYSNEKGLKNDDKSISLIKNFLDAIKEIEKFIKPLS |
| | | ETNITGEKNDLFYSQFTPLLDNISRIDILYDKVRNYVTQKPFSTDKIKL |
| | | NFGNYQLLNGWDKDKEREYGAVLLCKDEKYYLAIIDKSNNRILENIDFQ |
| | | DCDESDCYEKIIYKLLPTPNKMLPKVFFAKKHKKLLSPSDEILKIYKNG |
| | | TFKKGDKFSLDDCHKLIDFYKESFKKYPKWLIYNFKFKKTNGYNDIREF |
| | | YNDVALQGYNISKMKIPTSFIDKLVDEGKIYLFQLYNKDFSPHSKGTPN |
| | | LHTLYFKMLFDERNLEDVVYRLNGEAEMFYRPASIKYDKPTHPKNTPIK |
| | | NKNTLNDKRASTFPYDLIKDKRYTKWQFSLHFPITMNFKDPDKAMINDD |
| | | VRNLLKSCNNNFIIGIDRGERNLLYVSVINSNGAIIYQHSLNIIGNKFK |
| | | GKTYETNYREKLATREKDRTEQRRNWKAIESIKELKEGYISQAVHVICQ |
| | | LVVKYDAIIVMEKLTDGFKRGRTKFEKQVYQKFEKMLIDKLNYYVDKKL |
| | | DPDEEGGLLHAYQLTNKLESFDKLGTQSGFIFYVRPDFTSKIDPVTGFV |
| | | NLLYPRYEKIDKAKDMISRFDDIRYNAGEDFFEFDIDYDKFPKTASDYR |
| | | KKWTICINGERIEAFRNPANNNEWSYRTIILAEKFKELEDNNSINYRDS |
| | | DDLKAEILSQTKGKFFEDFFKLLRLTLQMRNSNPETGEDRILSPVKDKN |
| | | GNFYDSSKYDEKSKLPCDADANGAYNIARKGLWIVEQFKKADNVSTVEP |
| | | VIHNDKWLKFVQENDMANN |
| ART32 | 32 | KTGLDKLKDYAEIYYHCNTDADRKRLNKCASELRKEIVKNFKNRDEYNK |
| | | LFDKRMIEIVLPKHLKNEDEKEVVASFKNFTTYFTGFFTNRKNMYSDGE |
| | | ESTAIAYRCINENLPKHLDNVKAFEKAISKLSKNAIDDLDATYSGLCGT |
| | | NLYDVFTVDYFNFLLPQSGITEYNKIIGGYTTSDGTKVKGINEYINLYN |
| | | QQVSKRDKIPNLQILYKQILSESEKVSFIPPKFEDDNELLSAVSEFYAN |
| | | DETFDEMPLKKAIDETKLLFGNLDNSSLNGIYIQNDRSVINLSNSMEGS |
| | | WSVIEDLWNKNYDSVNSNSRIKDIQKREDKRKKAYKAEKKLSLSFLQVL |
| | | ISNSENNEIREKSIVDYYKTSLMQLTDNLSDKYNEVAPLLNENYSNEKG |
| | | LKNDDKSISLIKNFLDAIKEIEKFIKPLSETNITGEKNDLFYSQFTPLL |
| | | DNISRIDILYDKVRNYVTQKPFSTDKIKLNFGNYQLLNGWDKDKEREYG |
| | | AVLLCRDEKYYLAIIDKSNNRILENIDFQDCDESDCYEKIIYKLLPTPN |
| | | KMLPKVFFAKKHKKLLSPSDEILKIRKNGTFKKGDKFSLDDCHKLIDFY |
| | | KESFKKYPNWLIYNFKFKKTNEYNDIREFYNDVALQGYNISKMKIPTSF |
| | | IDKLVDEGKIYLFQLYNKDFSPHSKGTPNLHTLYFKMLFDERNLEDVVY |
| | | KLNGEAKMFYRPASIKYDKPTHPKNTPIKNKNTLNDKKASTFPYDLIKD |
| | | KRYTKWQFSLHFSITMNFKAPDKAMINDDVRNLLKSCNNNFIIGIDRGE |
| | | RNLLYVSVIDSNGAIIYQHSLNIIGNKFKGKTYETNYREKLATREKERT |
| | | EQRRNWKAIESIKELKEGYISQAVHVICQLVVKYDAIIVMEKLTDGEKR |
| | | GRTKFEKQVYQKFEKMLIDKLNYYVDKKLDPDEEGGLLHAYQLTNKLES |
| | | FDKLGTQSGFIFYVRPDFTSKIDPVTGFVNLLYPRYENIDKAKDMISRF |
| | | DDIRYNAGEDFFEFDIDYDKFPKTASDYRKKWTICTNGERIEAFRNPAN |
| | | NNEWSYRTIILAEKFKELFDNNSINYRDSDDLKAEILSQTKGKFFEDFF |
| | | KLLRLTLQMRNSNPETGEDRILSPVKDKNGNFYDSSKYDEKSKLPCDAD |
| | | ANGAYNIARKGLWIVEQFKKSDNVSTVEPVIHNDKWLKFVQENDMANN |
| ART33 | 33 | MSININKFSDECRKIDFFTDLYNIQKTLRFSLIPIGATADNFEFKGRLS |
| | | KEKDLLDSAKRIKEYISKYLADESDICLSQPVKLKHLDEYYELYITKDR |
| | | DEQKFKSVEEKLRKELADLLKEILKRLNKKILSDYLPEYLEDDEKALED |
| | | IANLSSFSTYFNSYYDNCKNMYTDKEQSTAIPYRCINDNLPKFIDNMKA |
| | | YEKALEELKPSDLEELRNNFKGVYDTTVDDMFTLDYFNCVLSQSGIDSY |
| | | NAIIGNDKVKGINEYINLHNQTAEQGHKVPNLKRLYKQIGSQKKTISFL |
| | | PSKFESDNELLKAVYDFYNTGDAEKNFTALKDTITEFEKIFDNLSEYNL |
| | | DGVFVRNDISLTNLSQSMFNDWSVFRNLWNDQYDKVNNPEKAKDIDKYN |
| | | DKRHKVYKKSESFSINQLQELIATTLEEDINSKKITDYFSCDFHRVTTE |
| | | VENKYQLVKDLLSSDYPKNKNLKTSEEDVALIKDELDSVKSLESFVKIL |
| | | TGTGKESGKDELFYGSFTKWFDQLRYIDKLYDKVRNYITEKPYSLDKIK |
| | | LSFDNPQFLGGWQHSKETDYSAQLFMKDGLYYLGVMDKETKREFKTQYN |
| | | TPENDSDTMVKIEYNQIPNPGRVIQNLMLVDGKIVKKNGRKNADGVNAV |
| | | LEELKNQYLPENINRIRKTESYKTTSNNENKDDLKAYLEYYIARTKEYY |
| | | CKYNFVFKSADEYGSFNEFVDDVNNQAYQITKVKVSEKQLLSLVEQGKL |
| | | YLFKIYNKDFSEYSKGKKNLHTMYFQMLEDDRNLENLVYKLQGGAEMFY |
| | | RPASIKKDSEFKHDANVEIIKRTCEDKVNDKDNPTDDEKAKYYSKFDYD |
| | | IVKNKRFTKDQFSLHLTLAMNCNQPDHYWLNNDVRELLKKSNKNHIIGI |
| | | DRGERNLIYVTIINSDGVIVDQINFNIIENSYNGKKYKTDYQKKLNQRE |
| | | EDRQKARKTWKTIETIKELKDGYISQVVHQICKLIVQYDAIVVMENING |
| | | GFKRGRTKVEKQVYQKFETMLINKLNYYVDKGTDYKECGGLLKAYQLTN |
| | | KFETFERIGKQSGIIFYVDPYLTSKIDPVTGFANLLYPKYETIPKTHNF |
| | | ISNIDDIRYNQSEDYFEFDIDYDKFPQGSYNYRKKWTICSYGNRIKYYK |
| | | DSRNKTASVVVDITEKFKETFTNAGIDFVNDNIKEKLLLVNSKELLKSF |
| | | MDTLKLTVQLRNSEINSDVDYIISPIKDRNGNFYYSENYKKSNNEVPSQ |
| | | PQDGDANGAYNIARKGLMIINKLKKADDVINNELLKISKKEWLEFAQKG |
| | | DLGE |
| ART34 | 34 | MKATSIWDNFTRKYSVSKTLRFELRPVGKTEENIVKKEIIDAEWISGKN |
| | | IPKGTDADRARDYKIVKKLLNQLHILFINQALSSENVKEFEKEDKKSKT |
| | | FVAWSDLLATHEDNWIQYTRDKSNSTVLKSLEKSKKDLYSKLGKLLNSK |
| | | ANAWKAEFISYHKIKSPDNIKIRLSASNVQILEGNTSDPIQLLKYQIEL |
| | | DNIKFLKDDGSEYTTKELADLLSTFEKFGTYESGFNQNRANVYDIDGEI |
| | | STSIAYRLFNQNIEFFFQNIKRWEQFTSSIGHKEAKENLKLVQWDIQSK |
| | | LKELDMEIVQPRFNLKFEKLLTPQSFIYLLNQEGIDAFNTVLGGIPAEV |
| | | KAEKKQGVNELINLTRQKLNEDKRKFPSLQIMYKQIMSERKINFIDQYE |
| | | DDVEMLKEIQEFSNDWNEKKKRHSASSKEIKESAIAYIQREFHETEDSL |
| | | EERATVKEDFYLSEKSIQNLSIDIFGGYNTIHNLWYTEVEGMLKSGERP |
| | | LTRVEKEKLKKQEYISFAQIERLISKHSQQYLDSTPKEANDRSLFKEKW |
| | | KKTFKNGFKVSEYTNLKLNELISEGETFQKIDQETGKETTIKIPGLFES |
| | | YENAILVESIKNQSLGTNKKESVPSIKEYLDSCLRLSKFIESFLVNSKD |
| | | LKEDQSLDGCSDFQNTLTQWLNEEFDVFILYNKVRNHVTKKPGNTDKIK |
| | | INFDNATLLDGWDVDKEAANFGFLLKKADNYYLGIADSSFNQDLKYFNE |
| | | GERLDEIEKNRKNLEKEESKNISKIDQEKVKKYKEVIDDLKAISNLNKG |
| | | RYSKAFYKQSKFTTLIPKCTTQLNEVIEHFKKEDTDYRIENKKFAKPFI |
| | | ITKEVFLLNNTVYDTATKKFTLKIGEDEDTKGLKKFQIGYYRATDDKKG |
| | | YESALRNWITFCIEFTKSYKSCLNYNYSSLKSVSEYKSLDEFYKDLNGI |
| | | GYTIDFVDISEEYINKKINEGKLYLFQIYNKDFSEKSKGKENLHTTYWK |
| | | LLFDSKNLEDVVIKLNGQAEVFFRPASIHEKEKITHFKNQEIQNKNPNA |
| | | VKKTSKFEYDIIKDNRFTKNKFLFHCPITLNFKADGNPYVNNEVQENIA |
| | | KNPNVNIIGIDRGEKHLLYFTVINQQGQILDAGSLNSIKSEYKDKNQQS |
| | | VSFETPYHKILDKKESERKEARESWQEIENIKELKAGYLSHVVHQLSNL |
| | | IVKYNAIVVLEDLNKGFKRGRFKVEKQVYQKFEKSLIEKLNYLVEKDRK |
| | | ESNEPGHHLNAYQLTNKELSFERLGKQSGVLFYATASYTSKVDPVTGFM |
| | | QNIYDPYHKEKTREFYKNFTKIVYNGNYFEFNYDLNSVKPDSEEKRYRT |
| | | NWTVCSCVIRSEYDSNSKTQKTYNVNDQLVKLFEDAKIKIENGNDLKST |
| | | ILEQDDKFIRDLHFYFIAIQKMRVVDSKIEKGEDSNDYIQSPVYPFYCS |
| | | KEIQPNKKGFYELPSNGDSNGAYNIARKGIVILDKIRLRVQIEKLFEDG |
| | | TKIDWQKLPNLISKVKDKKLLMTVFEEWAELTHQGEVQQGDLLGKKMSK |
| | | KGEQFAEFIKGLNVTKEDWEIYTQNEKVVQKQIKTWKLESNST |
| ART35 | 35 | MKAINEYYKQLGAYCREEGKEKDDFFKRIDGAYCAISHLFFGEHGEIAQ |
| | | SDSDVELIQKLLEAYKGLQRFIKPLLGHGDEADKDNEFDAKLRKVWDEL |
| | | DIITPLYDKVRNWLSRKIYNPEKIKLCFENNGKLLSGWVDSRTKSDNGT |
| | | QYGGYIFRKKNEIGEYDFYLGISADTKLFRRDAAISYDDGMYERLDYYQ |
| | | LKSKTLLGNSYVGDYGLDSMNLLSAFKNAAVKFQFEKEVVPKDKENVPK |
| | | YLKRLKLDYAGFYQILMNDDKVVDAYKIMKQHILATLTSSIRVPAAIEL |
| | | ATQKELGIDELIDEIMNLPSKSFGYFPIVTAAIEEANKRENKPLFLFKM |
| | | SNKDLSYAATASKGLRKGRGTENLHSMYLKALLGMTQSVEDIGSGMVFF |
| | | RHQTKGLAETTARHKANEFVANKNKLNDKKKSIFGYEIVKNKRFTVDKY |
| | | LFKLSMNLNYSQPNNNKIDVNSKVREIISNGGIKNIIGIDRGERNLLYL |
| | | SLIDLKGNIVMQKSLNILKDDHNAKETDYKGLLTEREGENKEARRNWKK |
| | | IANIKDLKRGYLSQVVHIISKMMVEYNAIVVLEDLNPGFIRGRQKIERN |
| | | VYEQFERMLIDKLNFYVDKHKGANETGGLLHALQLTSEFKNFKKSEHQN |
| | | GCLFYIPAWNTSKIDPATGFVNLFNTKYTNAVEAQEFFSKEDEIRYNEE |
| | | KDWFEFEFDYDKFTQKAHGTRTKWTLCTYGMRLRSFKNSAKQYNWDSEV |
| | | VALTEEFKRILGEAGIDIHENLKDAICNLEGKSQKYLEPLMQFMKLLLQ |
| | | LRNSKAGTDEDYILSPVADENGIFYDSRSCGDQLPENADANGAYNIARK |
| | | GLMLIEQIKNAEDLNNVKFDISNKAWLNFAQQKPYKNGMKAINEYYKQL |
| | | GAYCREEGKEKDDFFKRIDGAYCAISHLFFGEHGEIAQSDSDVELIQKL |
| | | LEAYKGLQRFIKPLLGHGDEADKDNEFDAKLRKVWDELDIITPLYDKVR |
| | | NWLSRKIYNPEKIKLCFENNGKLLSGWVDSRTKSDNGTQYGGYIFRKKN |
| | | EIGEYDFYLGISADTKLFRRDAAISYDDGMYERLDYYQLKSKTLLGNSY |
| | | VGDYGLDSMNLLSAFKNAAVKFQFEKEVVPKDKENVPKYLKRLKLDYAG |
| | | FYQILMNDDKVVDAYKIMKQHILATLTSSIRVPAAIELATQKELGIDEL |
| | | IDEIMNLPSKSFGYFPIVTAAIEEANKRENKPLFLFKMSNKDLSYAATA |
| | | SKGLRKGRGTENLHSMYLKALLGMTQSVEDIGSGMVFFRHQTKGLAETT |
| | | ARHKANEFVANKNKLNDKKKSIFGYEIVKNKRFTVDKYLFKLSMNLNYS |
| | | QPNNNKIDVNSKVREIISNGGIKNIIGIDRGERNLLYLSLIDLKGNIVM |
| | | QKSLNILKDDHNAKETDYKGLLTEREGENKEARRNWKKIANIKDLKRGY |
| | | LSQVVHIISKMMVEYNAIVVLEDLNPGFIRGRQKIERNVYEQFERMLID |
| | | KLNFYVDKHKGANETGGLLHALQLTSEFKNFKKSEHQNGCLFYIPAWNT |
| | | SKIDPATGFVNLFNTKYTNAVEAQEFFSKFDEIRYNEEKDWFEFEFDYD |
| | | KFTQKAHGTRTKWTLCTYGMRLRSFKNSAKQYNWDSEVVALTEEFKRIL |
| | | GEAGIDIHENLKDAICNLEGKSQKYLEPLMQFMKLLLQLRNSKAGTDED |
| | | YILSPVADENGIFYDSRSCGDQLPENADANGAYNIARKGLMLIEQIKNA |
| | | EDLNNVKFDISNKAWLNFAQQKPYKNG |
| ART11* | 36 | MYYQGLTKLYPISKTIRNELIPVGKTLEHIRMNNILEADIQRKSDYERV |
| | | KKLMDDYHKQLINESLQDVHLSYVEEAADLYLNASKDKDIVDKESKCQD |
| | | KLRKEIVNLLKSHENFPKIGNKEIIKLLQSLSDTEKDYNALDSFSKFYT |
| | | YFTSYNEVRKNLYSDEEKSSTAAYRLINENLPKFLDNIKAYSIAKSAGV |
| | | RAKELTEEEQDCLEMTETFERTLTQDGIDNYNELIGKLNFAINLYNQQN |
| | | NKLKGFRKVPKMKELYKQILSEREASFVDEFVDDEALLTNVESFSAHIK |
| | | EFLESDSLSRFAEVLEESGGEMVYIKNDTSKTTFSNIVFGSWNVIDERL |
| | | AEEYDSANSKKKKDEKYYDKRHKELKKNKSYSVEKIVSLSTETEDVIGK |
| | | YIEKLQADIIAIKETREVFEKVVLKEHDKNKSLRKNTKAIEAIKSELDT |
| | | IKDFERDIKLISGSEHEMEKNLAVYAEQENILSSIRNVDSLYNMSRNYL |
| | | TQKPFSTEKFKLNFNRATLLNGWDKNKETDNLGILLVKEGKYYLGIMNT |
| | | KANKSFVNPPKPKTDNVYHKVNYKLLPGPNKMLPKVFFAKSNLEYYKPS |
| | | EDLLAKYQAGTHKKGENFSLEDCHSLISFFKDSLEKHPDWSEFGFKFSD |
| | | TKKYDDLSGFYREVEKQGYKITYTDIDVEYIDSLVEKDELYFFQIYNKD |
| | | FSPYSKGNYNLHTLYLTMLFDERNLRNVVYKLNGEAEVFYRPASIGKDE |
| | | LIIHKSGEEIKNKNPKRAIDKPTSTFEYDIVKDRRYTKDKFMLHIPVTM |
| | | NFGVDETRRENEVVNDAIRGDDKVRVIGIDRGERNLLYVVVVDSDGTIL |
| | | EQISLNSIINNEYSIETDYHKLLDEKEGDRDRARKNWTTIENIKELKEG |
| | | YLSQVVNVIAKLVLKYDAIICLEDLNFGFKRGRQKVEKQVYQKFEKMLI |
| | | DKLNYLVIDKSRSQENPEEVGHVLNALQLTSKFTSFKELGKQTGIIYYV |
| | | PAYLTSKIDPTTGFANLFYVKYESVEKSKDFFNREDSICENKVAGYFEF |
| | | SFDYKNFTDRACGMRSKWKVCTNGERIIKYRNEEKNSSFDDKVIVLTEE |
| | | FKKLFNEYGIAFNDCMDLTDAINAIDDASFFRKLTKLFQQTLQMRNSSA |
| | | DGSRDYIISPVENDNGEFFNSEKCDKSKPKDADANGAFNIARKGLWVLE |
| | | QLYNSSSGEKLNLAMTNAEWLEYAQQHTI |

In certain embodiments, a Cas nuclease comprises ABW1 (SEQ ID NO: 3), ABW2 (SEQ ID NO: 16), ABW3 (SEQ ID NO: 29), ABW4 (SEQ ID NO: 42), ABW5 (SEQ ID NO: 55), ABW6 (SEQ ID NO: 68), ABW7 (SEQ ID NO: 81), ABW8 (SEQ ID NO: 94), or ABW9 (SEQ ID NO: 107) (all SEQ ID NOs for ABW1-9 and variants thereof from International (PCT) Application Publication No. WO 2021/108324), or variants thereof, such as any one of variants 1-10 of ABW1 (SEQ ID NOs: 4-13, respectively), any one of variants 1-10 of ABW2 (SEQ ID NOs: 17-26, respectively), any one of variants 1-10 of ABW3 (SEQ ID NOs: 30-39, respectively), any one of variants 1-10 of ABW4 (SEQ ID NOs: 43-52, respectively), any one of variants 1-10 of ABW5 (SEQ ID NOs: 56-65, respectively), any one of variants 1-10 of ABW6 (SEQ ID NOs: 69-78, respectively), any one of variants 1-10 of ABW7 (SEQ ID NOs: 82-91, respectively), any one of variants 1-10 of ABW8 (SEQ ID NOs: 95-104, respectively), any one of variants 1-10 of ABW9 (SEQ ID NOs: 108-117, respectively). ABW1-ABW9, and variants thereof are known in the art and are described in International (PCT) Application Publication No. WO 2021/108324.

More type V-A Cas nucleases and their corresponding naturally occurring CRISPR-Cas systems can be identified by computational and experimental methods known in the art, e.g., as described in U.S. Pat. No. 9,790,490 and Shmakov et al. (2015) MOL. CELL, 60:385. Exemplary computational methods include analysis of putative Cas proteins by homology modeling, structural BLAST, PSI-BLAST, or HHPred, and analysis of putative CRISPR loci by identification of CRISPR arrays. Exemplary experimental methods include in vitro cleavage assays and in-cell nuclease assays (e.g., the Surveyor assay) as described in Zetsche et al. (2015) CELL, 163:759.

In certain embodiments, the Cas protein is a Cas nuclease that directs cleavage of one or both strands at the target locus, such as the target strand (i.e., the strand having the target nucleotide sequence that is at least partially complementary to and can hybridize with a single guide nucleic acid or dual guide nucleic acids) and/or the non-target strand. In certain embodiments, the Cas nuclease directs cleavage of one or both strands within about 1, 2, 3, 4, 5, 6, 7, 8, 9, 10, 15, 20, 25, 50, 100, 200, 500, or more nucleotides from the first or last nucleotide of the target nucleotide sequence or its complementary sequence. In certain embodiments, the cleavage is staggered, i.e., generating sticky ends. In certain embodiments, the cleavage generates a staggered cut with a 5′ overhang. In certain embodiments, the cleavage generates a staggered cut with a 5′ overhang of 1 to 5 nucleotides, e.g., of 4 or 5 nucleotides. In certain embodiments, the cleavage site is distant from the PAM, e.g., the cleavage occurs after the 18th nucleotide on the non-target strand and after the 23rd nucleotide on the target strand.

In certain embodiments, a composition provided herein comprises a Cas nuclease that a compatible guide nucleic acid (gNA), e.g., a gRNA, is capable of activating. In certain embodiments, a composition provided herein further comprises a Cas protein that is related to the Cas nuclease that a compatible guide nucleic acid (gNA), e.g., a gRNA, is capable of activating. For example, in certain embodiments, a Cas protein comprises an amino acid sequence at least 80% (e.g., at least 85%, at least 90%, at least 91%, at least 92%, at least 93%, at least 94%, at least 95%, at least 96%, at least 97%, at least 98%, or at least 99%) identical to the Cas nuclease amino acid sequence. In certain embodiments, a Cas protein comprises a nuclease-inactive mutant of the Cas nuclease. In certain embodiments, a Cas protein further comprises an effector domain.

In certain embodiments, a Cas protein lacks substantially all DNA cleavage activity. Such a Cas protein can be generated, e.g., by introducing one or more mutations to an active Cas nuclease (e.g., a naturally occurring Cas nuclease). A mutated Cas protein is considered to lack substantially all DNA cleavage activity when the DNA cleavage activity of the protein has no more than about 25%, 10%, 5%, 1%, 0.1%, 0.01%, or less of the DNA cleavage activity of the corresponding non-mutated form, for example, nil or negligible as compared with the non-mutated form. Thus, a Cas protein may comprise one or more mutations (e.g., a mutation in the RuvC domain of a type V-A Cas protein) and be used as a generic DNA binding protein with or without fusion to an effector domain. Exemplary mutations include D908A, E993A, and D1263A with reference to the amino acid positions in AsCpf1; D832A, E925A, and D1180A with reference to the amino acid positions in LbCpf1; and D917A, E1006A, and D1255A with reference to the amino acid position numbering of the FnCpf1. More mutations can be designed and generated according to the crystal structure described in Yamano et al. (2016) CELL, 165: 949.

It is understood that a Cas protein, rather than losing nuclease activity to cleave all DNA, may lose the ability to cleave only the target strand or only the non-target strand of a double-stranded DNA, thereby being functional as a nickase (see, Gao et al. (2016) CELL RES., 26:901). Accordingly, in certain embodiments, a Cas nuclease is a Cas nickase. In certain embodiments, a Cas nuclease has the activity to cleave the non-target strand but lacks substantially the activity to cleave the target strand, e.g., by a mutation in the Nuc domain. In certain embodiments, a Cas nuclease has the cleavage activity to cleave the target strand but lacks substantially the activity to cleave the non-target strand.

In certain embodiments, a Cas nuclease has the activity to cleave a double-stranded DNA and result in a double-strand break.

Cas proteins that lack substantially all DNA cleavage activity or have the ability to cleave only one strand may also be identified from naturally occurring systems. For example, certain naturally occurring CRISPR-Cas systems may retain the ability to bind the target nucleotide sequence but lose entire or partial DNA cleavage activity in eukaryotic (e.g., mammalian or human) cells. Such type V-A proteins are disclosed, for example, in Kim et al. (2017) ACS SYNTH. BIOL. 6 (7): 1273-82 and Zhang et al. (2017) CELL DISCOV. 3:17018.

The activity of a Cas protein (e.g., Cas nuclease) can be altered, e.g., by creating an engineered Cas protein. In certain embodiments, altered activity of an engineered Cas protein comprises increased targeting efficiency and/or decreased off-target binding. While not wishing to be bound by theory, it is hypothesized that off-target binding can be recognized by the Cas protein, for example, by the presence of one or more mismatches between the spacer sequence and the target nucleotide sequence, which may affect the stability and/or conformation of the CRISPR-Cas complex. In certain embodiments, altered activity comprises modified binding, e.g., increased binding to the target locus (e.g., the target strand or the non-target strand) and/or decreased binding to off-target loci. In certain embodiments, altered activity comprises altered charge in a region of the protein that associates with a single guide nucleic acid or dual guide nucleic acids. In certain embodiments, altered activity of an engineered Cas protein comprises altered charge in a region of the protein that associates with the target strand and/or the non-target strand. In certain embodiments, altered activity of an engineered Cas protein comprises altered charge in a region of the protein that associates with an off-target locus. The altered charge can include decreased positive charge, decreased negative charge, increased positive charge, or increased negative charge. For example, decreased negative charge and increased positive charge may generally strengthen binding to the nucleic acid(s) whereas decreased positive charge and increased negative charge may weaken binding to the nucleic acid(s). In certain embodiments, altered activity comprises increased or decreased steric hindrance between the protein and a single guide nucleic acid or dual guide nucleic acids. In certain embodiments, altered activity comprises increased or decreased steric hindrance between the protein and the target strand and/or the non-target strand. In certain embodiments, altered activity comprises increased or decreased steric hindrance between the protein and an off-target locus. In certain embodiments, a modification or mutation comprises one or more substitutions of Lys, His, Arg, Glu, Asp, Ser, Gly, and/or Thr. In certain embodiments, a modification or mutation comprises one or more substitutions with Gly, Ala, Ile, Glu, and/or Asp. In certain embodiments, modification or mutation comprises one or more amino acid substitutions in the groove between the WED and RuvC domain of the Cas protein (e.g., a type V-A Cas protein).

In certain embodiments, altered activity of an engineered Cas protein comprises increased nuclease activity to cleave the target locus. In certain embodiments, altered activity of an engineered Cas protein comprises decreased nuclease activity to cleave an off-target locus. In certain embodiments, altered activity of an engineered Cas protein comprises altered helicase kinetics. In certain embodiments, an engineered Cas protein comprises a modification that alters formation of the CRISPR complex.

In certain embodiments, a protospacer adjacent motif (PAM) or PAM-like motif directs binding of a Cas protein complex to a target locus. Many Cas proteins have PAM specificity. The precise sequence and length requirements for the PAM differ depending on the Cas protein used. PAM sequences are typically 2-5 base pairs in length and are adjacent to (but located on a different strand of target DNA from) the target nucleotide sequence. PAM sequences can be identified using any suitable method, such as testing cleavage, targeting, or modification of oligonucleotides having the target nucleotide sequence and different PAM sequences.

Exemplary PAM sequences are provided in Tables 2 and 3. In certain embodiments, a Cas protein comprises MAD7 and the PAM is TTTN, wherein N is A, C, G, or T. In certain embodiments, a Cas protein comprises MAD7 and the PAM is CTTN, wherein N is A, C, G, or T. In certain embodiments, a Cas protein comprises AsCpf1 and the PAM is TTTN, wherein N is A, C, G, or T. In certain embodiments, a Cas protein comprises FnCpf1 and the PAM is 5′ TTN, wherein N is A, C, G, or T. PAM sequences for certain other type V-A Cas proteins are disclosed in Zetsche et al. (2015) CELL, 163:759 and U.S. Pat. No. 9,982,279. Further, engineering of the PAM Interacting (PI) domain of a Cas protein may allow programing of PAM specificity, improve target site recognition fidelity, and/or increase the versatility of an engineered, non-naturally occurring system. Exemplary approaches to alter the PAM specificity of Cpf1 arc described in Gao et al. (2017) NAT. BIOTECHNOL., 35:789.

In certain embodiments, an engineered Cas protein comprises a modification that alters the Cas protein specificity in concert with modification to targeting range. Cas mutants can be designed to have increased target specificity as well as accommodating modifications in PAM recognition, for example by choosing mutations that alter PAM specificity (e.g., in the PI domain) and combining those mutations with groove mutations that increase (or if desired, decrease) specificity for the on-target locus versus off-target loci. The Cas modifications described herein can be used to counter loss of specificity resulting from alteration of PAM recognition, enhance gain of specificity resulting from alteration of PAM recognition, counter gain of specificity resulting from alteration of PAM recognition, or enhance loss of specificity resulting from alteration of PAM recognition.

In certain embodiments, an engineered Cas protein comprises one or more nuclear localization signal (NLS) motifs. In certain embodiments, an engineered Cas protein comprises at least 2 (e.g., at least 3, at least 4, at least 5, at least 6, at least 7, at least 8, at least 9, or at least 10) NLS motifs. Non-limiting examples of NLS motifs include: the NLS of SV40 large T-antigen, having the amino acid sequence of PKKKRKV (SEQ ID NO: 40); the NLS from nucleoplasmin, e.g., the nucleoplasmin bipartite NLS having the amino acid sequence of KRPAATKKAGQAKKKK (SEQ ID NO: 41); the c-myc NLS, having the amino acid sequence of PAAKRVKLD (SEQ ID NO: 42) or RQRRNELKRSP (SEQ ID NO: 43); the hRNPA1 M9 NLS, having the amino acid sequence of NQSSNFGPMKGGNFGGRSSGPYGGGGQYFAKPRNQGGY (SEQ ID NO: 44); the importin-a IBB domain NLS, having the amino acid sequence of RMRIZFKNKGKDTAELRRRRVEVSVELRKAKKDEQILKRRNV (SEQ ID NO: 45); the myoma T protein NLS, having the amino acid sequence of VSRKRPRP (SEQ ID NO: 46) or PPKKARED (SEQ ID NO: 47); the human p53 NLS, having the amino acid sequence of PQPKKKPL (SEQ ID NO: 48); the mouse c-abl IV NLS, having the amino acid sequence of SALIKKKKKMAP (SEQ ID NO: 49); the influenza virus NS1 NLS, having the amino acid sequence of DRLRR (SEQ ID NO: 50) or PKQKKRK (SEQ ID NO: 51); the hepatitis virus 8 antigen NLS, having the amino acid sequence of RKLKKKIKKL (SEQ ID NO: 52); the mouse Mx 1 protein NLS, having the amino acid sequence of REKKKFLKRR (SEQ ID NO: 53); the human poly (ADP-ribose) polymerase NLS, having the amino acid sequence of KRKGDEVDGVDEVAKKKSKK (SEQ ID NO: 54); the human glucocorticoid receptor NLS, having the amino acid sequence of RKCLQAGMNLEARKTKK (SEQ ID NO: 55), and synthetic NLS motifs such as PAAKKKKLD (SEQ ID NO: 56).

In general, the one or more NLS motifs are of sufficient strength to drive accumulation of the Cas protein in a detectable amount in the nucleus of a eukaryotic cell. The strength of nuclear localization activity may derive from the number of NLS motif(s) in the Cas protein, the particular NLS motif(s) used, the position(s) of the NLS motif(s), or a combination of these and/or other factors. In certain embodiments, an engineered Cas protein comprises at least 1 (e.g., at least 2, at least 3, at least 4, at least 5, at least 6, at least 7, at least 8, at least 9, or at least 10) NLS motif(s) at or near the N-terminus (e.g., within about 1, 2, 3, 4, 5, 10, 15, 20, 25, 30, 40, 50, or more amino acids along the polypeptide chain from the N-terminus). In certain embodiments, an engineered Cas protein comprises at least 1 (e.g., at least 2, at least 3, at least 4, at least 5, at least 6, at least 7, at least 8, at least 9, or at least 10) NLS motif(s) at or near the C-terminus (e.g., within about 1, 2, 3, 4, 5, 10, 15, 20, 25, 30, 40, 50, or more amino acids along the polypeptide chain from the C-terminus). In certain embodiments, an engineered Cas protein comprises at least 1 (e.g., at least 2, at least 3, at least 4, at least 5, at least 6, at least 7, at least 8, at least 9, or at least 10) NLS motif(s) at or near the C-terminus and at least 1 (e.g., at least 2, at least 3, at least 4, at least 5, at least 6, at least 7, at least 8, at least 9, or at least 10) NLS motif(s) at or near the N-terminus. In certain embodiments, the engineered Cas protein comprises one, two, or three NLS motifs at or near the C-terminus. In certain embodiments, the engineered Cas protein comprises one NLS motif at or near the N-terminus and one, two, or three NLS motifs at or near the C-terminus. In certain embodiments, the engineered Cas protein comprises a nucleoplasmin NLS at or near the C-terminus.

Detection of accumulation in the nucleus may be performed by any suitable technique. For example, a detectable marker may be fused to a nucleic acid-targeting protein, such that location within a cell may be visualized. Cell nuclei may also be isolated from cells, the contents of which may then be analyzed by any suitable process for detecting the protein, such as immunohistochemistry, Western blot, or enzyme activity assay. Accumulation in the nucleus may also be determined indirectly, such as by an assay that detects the effect of the nuclear import of a Cas protein complex (e.g., assay for DNA cleavage or mutation at the target locus, or assay for altered gene expression activity) as compared to a control not exposed to the Cas protein or exposed to a Cas protein lacking one or more of the NLS motifs.

A Cas protein may comprise a chimeric Cas protein, e.g., a Cas protein having enhanced function by being a chimera. Chimeric Cas proteins may be new Cas proteins containing fragments from more than one naturally occurring Cas protein or variants thereof. For example, fragments of multiple type V-A Cas homologs (e.g., orthologs) may be fused to form a chimeric Cas protein. In certain embodiments, a chimeric Cas protein comprises fragments of Cpf1 orthologs from multiple species and/or strains.

In certain embodiments, a Cas protein comprises one or more effector domains. The one or more effector domains may be located at or near the N-terminus of the Cas protein and/or at or near the C-terminus of the Cas protein. In certain embodiments, an effector domain comprised in the Cas protein is a transcriptional activation domain (e.g., VP64), a transcriptional repression domain (e.g., a KRAB domain or an SID domain), an exogenous nuclease domain (e.g., FokI), a deaminase domain (e.g., cytidine deaminase or adenine deaminase), or a reverse transcriptase domain (e.g., a high fidelity reverse transcriptase domain). Other activities of effector domains include but are not limited to methylase activity, demethylase activity, transcription release factor activity, translational initiation activity, translational activation activity, translational repression activity, histone modification (e.g., acetylation or demethylation) activity, single-stranded RNA cleavage activity, double-strand RNA cleavage activity, single-strand DNA cleavage activity, double-strand DNA cleavage activity, and nucleic acid binding activity.

In certain embodiments, a Cas protein comprises one or more protein domains that enhance homology-directed repair (HDR) and/or inhibit non-homologous end joining (NHEJ). Exemplary protein domains having such functions are described in Jayavaradhan et al. (2019) NAT. COMMUN. 10 (1): 2866 and Janssen et al. (2019) MOL. THER. NUCLEIC ACIDS 16:141-54. In certain embodiments, a Cas protein comprises a dominant negative version of p53-binding protein 1 (53BP1), for example, a fragment of 53BP1 comprising a minimum focus forming region (e.g., amino acids 1231-1644 of human 53BP1). In certain embodiments, a Cas protein comprises a motif that is targeted by APC-Cdh1, such as amino acids 1-110 of human Geminin, thereby resulting in degradation of the fusion protein during the HDR non-permissive G1 phase of the cell cycle.

In certain embodiments, a Cas protein comprises an inducible or controllable domain. Non-limiting examples of inducers or controllers include light, hormones, and small molecule drugs. In certain embodiments, a Cas protein comprises a light inducible or controllable domain. In certain embodiments, a Cas protein comprises a chemically inducible or controllable domain.

In certain embodiments, a Cas protein comprises a tag protein or peptide for ease of tracking and/or purification. Non-limiting examples of tag proteins and peptides include fluorescent proteins (e.g., green fluorescent protein (GFP), YFP, RFP, CFP, mCherry, tdTomato), HIS tags (e.g., 6×His tag (SEQ ID NO: 2044), or gly-6×His (SEQ ID NO: 2045); 8×His (SEQ ID NO: 2046), or gly-8×His (SEQ ID NO: 2047)), hemagglutinin (HA) tag, FLAG tag, 3×FLAG tag, and Myc tag.

In certain embodiments, a Cas protein is conjugated to a non-protein moiety, such as a fluorophore useful for genomic imaging. In certain embodiments, a Cas protein is covalently conjugated to the non-protein moiety. The terms “CRISPR-Associated protein,” “Cas protein,” “Cas,” “CRISPR-Associated nuclease,” and “Cas nuclease” are used herein to include such conjugates despite the presence of one or more non-protein moieties.

### B. Guide Nucleic Acids

A guide nucleic acid can be a single gNA (sgNA, e.g., sgRNA), in which the gNA is a single polynucleotide, or a dual gNA (e.g., dual gRNA), in which the gNA comprises two separate polynucleotides (these can in some cases be covalently linked, but not via a conventional internucleotide linkage). In certain embodiments, a single guide nucleic acid is capable of activating a Cas nuclease alone (e.g., in the absence of a tracrRNA).

In general, a gNA comprises a modulator nucleic acid and a targeter nucleic acid. In a sgNA the modulator and targeter nucleic acids are part of a single polynucleotide. In a dual gNA the modulator and targeter nucleic acids are separate, e.g., not joined by a conventional nucleotide linkage, such as not joined at all. The targeter nucleic acid comprises a spacer sequence and a targeter stem sequence. The modulator nucleic acid comprises a modulator stem sequence and, generally, further nucleotides, such as nucleotides comprising a 5′ tail. The modulator stem sequence and targeter stem sequence can each comprise any suitable number of nucleotides and are of sufficient complementarity that they can hybridize. In a single gNA there may be additional NTs between the targeter stem sequence and the modulator stem sequence; these can, in certain cases, form secondary structure, such as a loop.

In certain embodiments, the guide nucleic acid comprises a targeter nucleic acid that, in combination with a modulator nucleic acid, is capable of binding a Cas protein. In certain embodiments, the guide nucleic acid comprises a targeter nucleic acid that, in combination with a modulator nucleic acid, is capable of activating a Cas nuclease. In certain embodiments, the system further comprises the Cas protein that the targeter nucleic acid and the modulator nucleic acid are capable of binding or the Cas nuclease that the targeter nucleic acid and the modulator nucleic acid are capable of activating.

It is contemplated that the single or dual guide nucleic acids need to be the compatible with a Cas protein (e.g., Cas nuclease) to provide an operative CRISPR system. For example, the targeter stem sequence and the modulator stem sequence can be derived from a naturally occurring crRNA capable of activating a Cas nuclease in the absence of a tracrRNA.

Alternatively, the targeter stem sequence and the modulator stem sequence can be derived from a naturally occurring set of crRNA and tracrRNA, respectively, that are capable of activating a Cas nuclease. In certain embodiments, the nucleotide sequences of the targeter stem sequence and the modulator stem sequence are identical to the corresponding stem sequences of a stem-loop structure in such naturally occurring crRNA.

Guide nucleic acid sequences that are operative with a type II or type V Cas protein are known in the art and are disclosed, for example, in U.S. Pat. Nos. 9,790,490, 9,896,696, 10,113,179, and 10,266,850, and U.S. Patent Application Publication No. 2014/0242664. It is understood that these sequences are merely illustrative, and other guide nucleic acid sequences may also be used with these Cas proteins.

TABLE 4
Type V-A Cas Protein and Corresponding Single Guide Nucleic Acid Sequences

| Cas Protein | Scaffold Sequence¹ | PAM² |
|---|---|---|
| MAD7 (SEQ ID | UAAUUUCUACUCUUGUAGA (SEQ ID NO: 57), | 5′ TTTN |
| NO: 37) | AUCUACAACAGUAGA (SEQ ID NO: 58), | or 5′ |
| | AUCUACAAAAGUAGA (SEQ ID NO: 59), | CTTN |
| | GGAAUUUCUACUCUUGUAGA (SEQ ID NO: 60), | |
| | UAAUUCCCACUCUUGUGGG (SEQ ID NO: 61) | |
| MAD2 (SEQ ID | AUCUACAAGAGUAGA (SEQ ID NO: 62), | 5′ TTTN |
| NO: 38) | AUCUACAACAGUAGA (SEQ ID NO: 58), | |
| | AUCUACAAAAGUAGA (SEQ ID NO: 59), | |
| | AUCUACACUAGUAGA (SEQ ID NO: 63) | |
| AsCpf1 (SEQ | UAAUUUCUACUCUUGUAGA (SEQ ID NO: 57) | 5′ TTTN |
| ID NO: 3 of | | |
| WO | | |
| 2021/158918) | | |
| LbCpf1 (SEQ | UAAUUUCUACUAAGUGUAGA (SEQ ID NO: 64) | 5′ TTTN |
| ID NO: 4 of | | |
| WO | | |
| 2021/158918) | | |
| FnCpf1 (SEQ | UAAUUUUCUACUUGUUGUAGA (SEQ ID NO: 65) | 5′ TTN |
| ID NO: 5 of | | |
| WO | | |
| 2021/158918) | | |
| PbCpf1 (SEQ | AAUUUCUACUGUUGUAGA (SEQ ID NO: 66) | 5′ TTTC |
| ID NO: 6 of | | |
| WO | | |
| 2021/158918) | | |
| PsCpf1 (SEQ | AAUUUCUACUGUUGUAGA (SEQ ID NO: 66) | 5′ TTTC |
| ID NO: 7 of | | |
| WO | | |
| 2021/158918) | | |
| As2Cpf1 (SEQ | AAUUUCUACUGUUGUAGA (SEQ ID NO: 66) | 5′ TTTC |
| ID NO: 8 of | | |
| WO | | |
| 2021/158918) | | |
| McCpf1 (SEQ | GAAUUUCUACUGUUGUAGA (SEQ ID NO: 67) | 5′ TTTC |
| ID NO: 9 of | | |
| WO | | |
| 2021/158918) | | |
| Lb3Cpf1 (SEQ | GAAUUUCUACUGUUGUAGA (SEQ ID NO: 67) | 5′ TTTC |
| ID NO: 10 of | | |
| WO | | |
| 2021/158918) | | |
| EcCpf1 (SEQ | GAAUUUCUACUGUUGUAGA (SEQ ID NO: 67) | 5′ TTTC |
| ID NO: 11 of | | |
| WO | | |
| 2021/158918) | | |
| SmCsm1 (SEQ | GAAUUUCUACUGUUGUAGA (SEQ ID NO: 67) | 5′ TTTC |
| ID NO: 12 of | | |
| WO | | |
| 2021/158918) | | |
| SsCsm1 (SEQ | GAAUUUCUACUGUUGUAGA (SEQ ID NO: 67) | 5′ TTTC |
| ID NO: 13 of | | |
| WO | | |
| 2021/158918) | | |
| MbCsm1 (SEQ | GAAUUUCUACUGUUGUAGA (SEQ ID NO: 67) | 5′ TTTC |
| ID NO: 14 of | | |
| WO | | |
| 2021/158918) | | |
| ART2 (SEQ ID | GUCUAAAGGUACCACCAAAUUUCUACUGUUGUAGAU | 5′ TTTN |
| NO: 2 | (SEQ ID NO: 68) | or 5′ |
| | | NTTN |
| ART11 (SEQ ID | GCUUAGAACCUUUAAAUAAUUUCUACUAUUGUAGAU | 5′ TTTN |
| NO: 11 | (SEQ ID NO: 69) | or 5′ |
| | | NTTN |
| ART11* (SEQ | GCUUAGAACCUUUAAAUAAUUUCUACUAUUGUAGAU | 5′ TTTN |
| ID NO: 36 | (SEQ ID NO: 69) | or 5′ |
| | | NTTN |

¹The modulator sequence in the scaffold sequence is underlined; the targeter stem sequence in the scaffold sequence is bold-underlined. It is understood that a “scaffold sequence” listed herein constitutes a portion of a single guide nucleic acid. Additional nucleotide sequences, other than the spacer sequence, can be comprised in the single guide nucleic acid.
²In the consensus PAM sequences, N represents A, C, G, or T. Where the PAM sequence is preceded by “5′,” it means that the PAM is located immediately upstream of the target nucleotide sequence when using the non-target strand (i.e., the strand not hybridized with the spacer sequence) as the coordinate.

TABLE 5
Type V-A Cas Protein and Corresponding Dual Guide Nucleic Acid Sequences

| | | Targeter | |
|---|---|---|---|
| | | Stem | |
| Cas Protein | Modulator Sequence¹ | Sequence | PAM² |
| MAD7 (SEQ ID NO: | UAAUUUCUAC (SEQ ID NO: | GUAGA | 5′ TTTN |
| 37) | 70) | | or 5′ |
| | AUCUAC | GUAGA | CTTN |
| | GGAAUUUCUAC (SEQ ID NO: | GUAGA | |
| | 72) | | |
| | UAAUUCCCAC (SEQ ID NO: | GUGGG | |
| | 73) | | |
| MAD2 (SEQ ID NO: | AUCUAC | GUAGA | 5′ TTTN |
| 38) | | | |
| AsCpf1 (SEQ ID NO: | UAAUUUCUAC (SEQ ID NO: | GUAGA | 5′ TTTN |
| 3 of WO | 70) | | |
| 2021/158918) | | | |
| LbCpf1 (SEQ ID NO: | UAAUUUCUAC (SEQ ID NO: | GUAGA | 5′ TTTN |
| 4 of WO | 70) | | |
| 2021/158918) | | | |
| FnCpf1 (SEQ ID NO: | UAAUUUUCUACU (SEQ ID NO: | GUAGA | 5′ TTN |
| 5 of WO | 74) | | |
| 2021/158918) | | | |
| PbCpf1 (SEQ ID NO: | AAUUUCUAC | GUAGA | 5′ TTTC |
| 6 of WO | | | |
| 2021/158918) | | | |
| PsCpf1 (SEQ ID NO: | AAUUUCUAC | GUAGA | 5′ TTTC |
| 7 of WO | | | |
| 2021/158918) | | | |
| As2Cpf1 (SEQ ID | AAUUUCUAC | GUAGA | 5′ TTTC |
| NO: 8 of WO | | | |
| 2021/158918) | | | |
| McCpf1 (SEQ ID NO: | GAAUUUCUAC (SEQ ID NO: | GUAGA | 5′ TTTC |
| 9 of WO | 76) | | |
| 2021/158918) | | | |
| Lb3Cpf1 (SEQ ID | GAAUUUCUAC (SEQ ID NO: | GUAGA | 5′ TTTC |
| NO: 10 of WO | | | |
| 2021/158918) | 76) | | |
| EcCpf1 (SEQ ID NO: | GAAUUUCUAC (SEQ ID NO: | GUAGA | 5′ TTTC |
| 11 of WO | 76) | | |
| 2021/158918) | | | |
| SmCsm1 (SEQ ID NO: | GAAUUUCUAC (SEQ ID NO: | GUAGA | 5′ TTTC |
| 12 of WO | 76) | | |
| 2021/158918) | | | |
| SsCsm1 (SEQ ID NO: | GAAUUUCUAC (SEQ ID NO: | GUAGA | 5′ TTTC |
| 13 of WO | 76) | | |
| 2021/158918) | | | |
| MbCsm1 (SEQ ID NO: | GAAUUUCUAC (SEQ ID NO: | GUAGA | 5′ TTTC |
| 14 of WO | 76) | | |
| 2021/158918) | | | |
| ART2 (SEQ ID NO: 2) | AAAUUUCUAC (SEQ ID NO: | GUAGA | 5′ TTTN |
| | 77) | | or 5′ |
| | | | NTTN |
| ART11 (SEQ ID NO: | UAAUUUCUAC (SEQ ID NO: | GUAGA | 5′ TTTN |
| 11) | 70) | | or 5′ |
| | | | NTTN |
| ART11* (SEQ ID NO: | UAAUUUCUAC (SEQ ID NO: | GUAGA | 5′ TTTN |
| 36) | 70) | | or 5′ |
| | | | NTTN |

¹It is understood that a “modulator sequence” listed herein may constitute the nucleotide sequence of a modulator nucleic acid. Alternatively, additional nucleotide sequences can be comprised in the modulator nucleic acid 5′ and/or 3′ to a “modulator sequence” listed herein.
²In the consensus PAM sequences, N represents A, C, G, or T. Where the PAM sequence is preceded by “5′,” it means that the PAM is located immediately upstream of the target nucleotide sequence when using the non-target strand (i.e., the strand not hybridized with the spacer sequence) as the coordinate.

In certain embodiments, a guide nucleic acid, in the context of a type V-A CRISPR-Cas system, comprises a targeter stem sequence listed in Table 5. The same targeter stem sequences, as a portion of scaffold sequences, are bold-underlined in Table 4.

In certain embodiments, a guide nucleic acid is a single guide nucleic acid that comprises, from 5′ to 3′, a modulator stem sequence, a loop sequence, a targeter stem sequence, and a spacer sequence. In certain embodiments, the targeter stem sequence in the single guide nucleic acid is listed in Table 4 as a bold-underlined portion of scaffold sequence, and the modulator stem sequence is complementary (e.g., 100% complementary) to the targeter stem sequence. In certain embodiments, the single guide nucleic acid comprises, from 5′ to 3′, a modulator sequence listed in Table 4 as an underlined portion of a scaffold sequence, a loop sequence, a targeter stem sequence a bold-underlined portion of the same scaffold sequence, and a spacer sequence. In certain embodiments, an engineered, non-naturally occurring system comprises a single guide nucleic acid comprising a scaffold sequence listed in Table 4. In certain embodiments, the system further comprises a Cas protein (e.g., Cas nuclease) comprising an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in the SEQ ID NO listed in the same line of Table 4. In certain embodiments, the system further comprises a Cas protein (e.g., Cas nuclease) comprising the amino acid sequence set forth in the SEQ ID NO listed in the same line of Table 4. In certain embodiments, the system is useful for targeting, editing, or modifying a nucleic acid comprising a target nucleotide sequence close or adjacent to (e.g., immediately downstream of) a PAM listed in the same line of Table 4 when using the non-target strand (i.e., the strand not hybridized with the spacer sequence) as the coordinate.

In certain embodiments, a guide nucleic acid, e.g., dual gNA, comprises a targeter guide nucleic acid that comprises, from 5′ to 3′, a targeter stem sequence and a spacer sequence. In certain embodiments, the targeter stem sequence in the targeter nucleic acid is listed in Table 5. In certain embodiments, an engineered, non-naturally occurring system comprises the targeter nucleic acid and a modulator stem sequence complementary (e.g., 100% complementary) to the targeter stem sequence. In certain embodiments, the modulator nucleic acid comprises a modulator sequence listed in the same line of Table 5. In certain embodiments, the system further comprises a Cas protein (e.g., Cas nuclease) comprising an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in the SEQ ID NO listed in the same line of Table 5. In certain embodiments, the system further comprises a Cas protein (e.g., Cas nuclease) comprising the amino acid sequence set forth in the SEQ ID NO listed in the same line of Table 5. In certain embodiments, the system is useful for targeting, editing, or modifying a nucleic acid comprising a target nucleotide sequence close or adjacent to (e.g., immediately downstream of) a PAM listed in the same line of Table 5 when using the non-target strand (i.e., the strand not hybridized with the spacer sequence) as the coordinate.

A single guide nucleic acid, the targeter nucleic acid, and/or the modulator nucleic acid can be synthesized chemically or produced in a biological process (e.g., catalyzed by an RNA polymerase in an in vitro reaction). Such reaction or process may limit the lengths of the single guide nucleic acid, targeter nucleic acid, and/or modulator nucleic acid. In certain embodiments, a single guide nucleic acid is no more than 100, 90, 80, 70, 60, 50, 40, 30, or 25 nucleotides in length. In certain embodiments, a single guide nucleic acid is at least 20, 25, 30, 40, 50, 60, 70, 80, or 90 nucleotides in length. In certain embodiments, the single guide nucleic acid is 20-100, 20-90, 20-80, 20-70, 20-60, 20-50, 20-40, 20-30, 20-25, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length. In certain embodiments, a targeter nucleic acid is no more than 100, 90, 80, 70, 60, 50, 40, 30, or 25 nucleotides in length. In certain embodiments, a targeter nucleic acid is at least 20, 25, 30, 40, 50, 60, 70, 80, or 90 nucleotides in length. In certain embodiments, the targeter nucleic acid is 20-100, 20-90, 20-80, 20-70, 20-60, 20-50, 20-40, 20-30, 20-25, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length. In certain embodiments, a modulator nucleic acid is no more than 100, 90, 80, 70, 60, 50, 40, 30, or 20 nucleotides in length. In certain embodiments, a modulator nucleic acid is at least 10, 15, 20, 25, 30, 40, 50, 60, 70, 80, or 90 nucleotides in length. In certain embodiments, the modulator nucleic acid is 10-100, 10-90, 10-80, 10-70, 10-60, 10-50, 10-40, 10-30, 10-20, 15-100, 15-90, 15-80, 15-70, 15-60, 15-50, 15-40, 15-30, 15-20, 20-100, 20-90, 20-80, 20-70, 20-60, 20-50, 20-40, 20-30, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length.

It is contemplated that the length of the duplex formed within the single guide nuclei acid or formed between the targeter nucleic acid and the modulator nucleic acid, e.g., in a dual gNA, may be a factor in providing an operative CRISPR system. In certain embodiments, the targeter stem sequence and the modulator stem sequence each consist of 4-10 nucleotides that base pair with each other. In certain embodiments, the targeter stem sequence and the modulator stem sequence each consist of 4-9, 4-8, 4-7, 4-6, 4-5, 5-10, 5-9, 5-8, 5-7, or 5-6 nucleotides that base pair with each other. In certain embodiments, the targeter stem sequence and the modulator stem sequence each consist of 4, 5, 6, 7, 8, 9, or 10 nucleotides. It is understood that the composition of the nucleotides in each sequence affects the stability of the duplex, and a C-G base pair confers greater stability than an A-U base pair. In certain embodiments, 20%-80%, 20%-70%, 20%-60%, 20%-50%, 20%-40%, 20%-30%, 30%-80%, 30%-70%, 30%-60%, 30%-50%, 30%-40%, 40%-80%, 40%-70%, 40%-60%, 40%-50%, 50%-80%, 50%-70%, 50%-60%, 60%-80%, 60%-70%, or 70%-80% of the base pairs are C-G base pairs.

In certain embodiments, the targeter stem sequence and the modulator stem sequence each consist of 5 nucleotides. As such, the targeter stem sequence and the modulator stem sequence form a duplex of 5 base pairs. In certain embodiments, 0-4, 0-3, 0-2, 0-1, 1-5, 1-4, 1-3, 1-2, 2-5, 2-4, 2-3, 3-5, 3-4, or 4-5 out of the 5 base pairs are C-G base pairs. In certain embodiments, 0, 1, 2, 3, 4, or 5 out of the 5 base pairs are C-G base pairs. In certain embodiments, the targeter stem sequence consists of 5′-GUAGA-3′ and the modulator stem sequence consists of 5′-UCUAC-3′. In certain embodiments, the targeter stem sequence consists of 5′-GUGGG-3′ and the modulator stem sequence consists of 5′-CCCAC-3′.

In certain embodiments, in a type V-A system, the 3′ end of the targeter stem sequence is linked by no more than 1, 2, 3, 4, 5, 6, 7, 8, 9, or 10 nucleotides to the 5′ end of the spacer sequence. In certain embodiments, the targeter stem sequence and the spacer sequence are adjacent to each other, directly linked by an internucleotide bond. In certain embodiments, the targeter stem sequence and the spacer sequence are linked by one nucleotide, e.g., a uridine. In certain embodiments, the targeter stem sequence and the spacer sequence are linked by two or more nucleotides. In certain embodiments, the targeter stem sequence and the spacer sequence are linked by 1, 2, 3, 4, 5, 6, 7, 8, 9, or 10 nucleotides.

In certain embodiments, the targeter nucleic acid further comprises an additional nucleotide sequence 5′ to the targeter stem sequence. In certain embodiments, the additional nucleotide sequence comprises at least 1 (e.g., at least 2, at least 3, at least 4, at least 5, at least 6, at least 7, at least 8, at least 9, at least 10, at least 15, at least 20, at least 25, at least 30, at least 35, at least 40, at least 45, or at least 50) nucleotides. In certain embodiments, the additional nucleotide sequence consists of 1, 2, 3, 4, 5, 6, 7, 8, 9, 10, 15, 20, 25, 30, 35, 40, 45, or 50 nucleotides. In certain embodiments, the additional nucleotide sequence consists of 2 nucleotides. In certain embodiments, the additional nucleotide sequence is reminiscent to the loop or a fragment thereof (e.g., one, two, three, or four nucleotides at the 3′ end of the loop) in a crRNA of a corresponding single guide CRISPR-Cas system. It is understood that an additional nucleotide sequence 5′ to the targeter stem sequence can be dispensable. Accordingly, in certain embodiments, the targeter nucleic acid does not comprise any additional nucleotide 5′ to the targeter stem sequence.

In certain embodiments, the targeter nucleic acid or the single guide nucleic acid further comprises an additional nucleotide sequence containing one or more nucleotides at the 3′ end that does not hybridize with the target nucleotide sequence. The additional nucleotide sequence may protect the targeter nucleic acid from degradation by 3′-5′ exonuclease. In certain embodiments, the additional nucleotide sequence is no more than 100 nucleotides in length. In certain embodiments, the additional nucleotide sequence is no more than 90, 80, 70, 60, 50, 40, 30, 20, or 10 nucleotides in length. In certain embodiments, the additional nucleotide sequence is at least 1, 2, 3, 4, 5, 6, 7, 8, 9, 10, 15, 20, 25, 30, 35, 40, 45, or 50 nucleotides in length. In certain embodiments, the additional nucleotide sequence is 5-100, 5-50, 5-40, 5-30, 5-25, 5-20, 5-15, 5-10, 10-100, 10-50, 10-40, 10-30, 10-25, 10-20, 10-15, 15-100, 15-50, 15-40, 15-30, 15-25, 15-20, 20-100, 20-50, 20-40, 20-30, 20-25, 25-100, 25-50, 25-40, 25-30, 30-100, 30-50, 30-40, 40-100, 40-50, or 50-100 nucleotides in length.

In certain embodiments, the additional nucleotide sequence forms a hairpin with the spacer sequence. Such secondary structure may increase the specificity of guide nucleic acid or the engineered, non-naturally occurring system (see, Kocak et al. (2019) NAT. BIOTECH. 37:657-66). In certain embodiments, the free energy change during the hairpin formation is greater than or equal to −20 kcal/mol, −15 kcal/mol, −14 kcal/mol, −13 kcal/mol, −12 kcal/mol, −11 kcal/mol, or −10 kcal/mol. In certain embodiments, the free energy change during the hairpin formation is greater than or equal to −5 kcal/mol, −6 kcal/mol, −7 kcal/mol, −8 kcal/mol, −9 kcal/mol, −10 kcal/mol, −11 kcal/mol, −12 kcal/mol, −13 kcal/mol, −14 kcal/mol, or −15 kcal/mol. In certain embodiments, the free energy change during the hairpin formation is in the range of −20 to −10 kcal/mol, −20 to −11 kcal/mol, −20 to −12 kcal/mol, −20 to −13 kcal/mol, −20 to −14 kcal/mol, −20 to −15 kcal/mol, −15 to −10 kcal/mol, −15 to −11 kcal/mol, −15 to −12 kcal/mol, −15 to −13 kcal/mol, −15 to −14 kcal/mol, −14 to −10 kcal/mol, −14 to −11 kcal/mol, −14 to −12 kcal/mol, −14 to −13 kcal/mol, −13 to −10 kcal/mol, −13 to −11 kcal/mol, −13 to −12 kcal/mol, −12 to −10 kcal/mol, −12 to −11 kcal/mol, or −11 to −10 kcal/mol. In other embodiments, the targeter nucleic acid or the single guide nucleic acid does not comprise any nucleotide 3′ to the spacer sequence.

In certain embodiments, the modulator nucleic acid further comprises an additional nucleotide sequence 3′ to the modulator stem sequence. In certain embodiments, the additional nucleotide sequence comprises at least 1 (e.g., at least 2, at least 3, at least 4, at least 5, at least 6, at least 7, at least 8, at least 9, at least 10, at least 15, at least 20, at least 25, at least 30, at least 35, at least 40, at least 45, or at least 50) nucleotides. In certain embodiments, the additional nucleotide sequence consists of 1, 2, 3, 4, 5, 6, 7, 8, 9, 10, 15, 20, 25, 30, 35, 40, 45, or 50 nucleotides. In certain embodiments, the additional nucleotide sequence consists of 1 nucleotide (e.g., uridine). In certain embodiments, the additional nucleotide sequence consists of 2 nucleotides. In certain embodiments, the additional nucleotide sequence is reminiscent to the loop or a fragment thereof (e.g., one, two, three, or four nucleotides at the 5′ end of the loop) in a crRNA of a corresponding single guide CRISPR-Cas system. It is understood that an additional nucleotide sequence 3′ to the modulator stem sequence can be dispensable. Accordingly, in certain embodiments, the modulator nucleic acid does not comprise any additional nucleotide 3′ to the modulator stem sequence.

It is understood that the additional nucleotide sequence 5′ to the targeter stem sequence and the additional nucleotide sequence 3′ to the modulator stem sequence, if present, may interact with each other. For example, although the nucleotide immediately 5′ to the targeter stem sequence and the nucleotide immediately 3′ to the modulator stem sequence do not form a Watson-Crick base pair (otherwise they would constitute part of the targeter stem sequence and part of the modulator stem sequence, respectively), other nucleotides in the additional nucleotide sequence 5′ to the targeter stem sequence and the additional nucleotide sequence 3′ to the modulator stem sequence may form one, two, three, or more base pairs (e.g., Watson-Crick base pairs). Such interaction may affect the stability of a complex comprising the targeter nucleic acid and the modulator nucleic acid.

The stability of a complex comprising a targeter nucleic acid and a modulator nucleic acid can be assessed by the Gibbs free energy change (AG) during the formation of the complex, either calculated or actually measured. Where all the predicted base pairing in the complex occurs between a base in the targeter nucleic acid and a base in the modulator nucleic acid, i.e., there is no intra-strand secondary structure, the AG during the formation of the complex correlates generally with the AG during the formation of a secondary structure within the corresponding single guide nucleic acid. Methods of calculating or measuring the AG are known in the art. An exemplary method is RNAfold (rna.tbi.univie.ac.at/cgi-bin/RNA WebSuite/RNAfold.cgi) as disclosed in Gruber et al. (2008) NUCLEIC ACIDS RES., 36 (Web Server issue): W70-W74. Unless indicated otherwise, the AG values in the present disclosure are calculated by RNAfold for the formation of a secondary structure within a corresponding single guide nucleic acid. In certain embodiments, the AG is lower than or equal to −1 kcal/mol, e.g., lower than or equal to −2 kcal/mol, lower than or equal to −3 kcal/mol, lower than or equal to −4 kcal/mol, lower than or equal to −5 kcal/mol, lower than or equal to −6 kcal/mol, lower than or equal to −7 kcal/mol, lower than or equal to −7.5 kcal/mol, or lower than or equal to −8 kcal/mol. In certain embodiments, the AG is greater than or equal to −10 kcal/mol, e.g., greater than or equal to −9 kcal/mol, greater than or equal to −8.5 kcal/mol, or greater than or equal to −8 kcal/mol. In certain embodiments, the AG is in the range of −10 to −4 kcal/mol. In certain embodiments, the AG is in the range of −8 to −4 kcal/mol, −7 to −4 kcal/mol, −6 to −4 kcal/mol, −5 to −4 kcal/mol, −8 to −4.5 kcal/mol, −7 to −4.5 kcal/mol, −6 to −4.5 kcal/mol, or −5 to −4.5 kcal/mol. In certain embodiments, the AG is about-8 kcal/mol, −7 kcal/mol, −6 kcal/mol, −5 kcal/mol, −4.9 kcal/mol, −4.8 kcal/mol, −4.7 kcal/mol, −4.6 kcal/mol, −4.5 kcal/mol, −4.4 kcal/mol, −4.3 kcal/mol, −4.2 kcal/mol, −4.1 kcal/mol, or −4 kcal/mol.

It is understood that the AG may be affected by a sequence in the targeter nucleic acid that is not within the targeter stem sequence, and/or a sequence in the modulator nucleic acid that is not within the modulator stem sequence. For example, one or more base pairs (e.g., Watson-Crick base pair) between an additional sequence 5′ to the targeter stem sequence and an additional sequence 3′ to the modulator stem sequence may reduce the AG, i.e., stabilize the nucleic acid complex. In certain embodiments, the nucleotide immediately 5′ to the targeter stem sequence comprises a uracil or is a uridine, and the nucleotide immediately 3′ to the modulator stem sequence comprises a uracil or is a uridine, thereby forming a nonconventional U-U base pair.

In certain embodiments, the modulator nucleic acid or the single guide nucleic acid comprises a nucleotide sequence referred to herein as a “5′ tail” positioned 5′ to the modulator stem sequence. In a naturally occurring type V-A CRISPR-Cas system, the 5′ tail is a nucleotide sequence positioned 5′ to the stem-loop structure of the crRNA. A 5′ tail in an engineered type V-A CRISPR-Cas system, whether single guide or dual guide, can be reminiscent to the 5′ tail in a corresponding naturally occurring type V-A CRISPR-Cas system.

Without being bound by theory, it is contemplated that the 5′ tail may participate in the formation of the CRISPR-Cas complex. For example, in certain embodiments, the 5′ tail forms a pseudoknot structure with the modulator stem sequence, which is recognized by the Cas protein (see, Yamano et al. (2016) CELL, 165:949). In certain embodiments, the 5′ tail is at least 3 (e.g., at least 4 or at least 5) nucleotides in length. In certain embodiments, the 5′ tail is 3, 4, or 5 nucleotides in length. In certain embodiments, the nucleotide at the 3′ end of the 5′ tail comprises a uracil or is a uridine. In certain embodiments, the second nucleotide in the 5′ tail, the position counted from the 3′ end, comprises a uracil or is a uridine. In certain embodiments, the third nucleotide in the 5′ tail, the position counted from the 3′ end, comprises an adenine or is an adenosine. This third nucleotide may form a base pair (e.g., a Watson-Crick base pair) with a nucleotide 5′ to the modulator stem sequence. Accordingly, in certain embodiments, the modulator nucleic acid comprises a uridine or a uracil-containing nucleotide 5′ to the modulator stem sequence. In certain embodiments, the 5′ tail comprises the nucleotide sequence of 5′-AUU-3′. In certain embodiments, the 5′ tail comprises the nucleotide sequence of 5′-AAUU-3′. In certain embodiments, the 5′ tail comprises the nucleotide sequence of 5′-UAAUU-3′. In certain embodiments, the 5′ tail is positioned immediately 5′ to the modulator stem sequence.

In certain embodiments, the single guide nucleic acid, the targeter nucleic acid, and/or the modulator nucleic acid are designed to reduce the degree of secondary structure other than the hybridization between the targeter stem sequence and the modulator stem sequence. In certain embodiments, no more than about 75%, 50%, 40%, 30%, 25%, 20%, 15%, 10%, 5%, 1%, or fewer of the nucleotides of the single guide nucleic acid other than the targeter stem sequence and the modulator stem sequence participate in self-complementary base pairing when optimally folded. In certain embodiments, no more than about 75%, 50%, 40%, 30%, 25%, 20%, 15%, 10%, 5%, 1%, or fewer of the nucleotides of the targeter nucleic acid and/or the modulator nucleic acid participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (NUCLEIC ACIDS RES. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106 (1): 23-24; and PA Carr and GM Church, 2009, Nature Biotechnology 27 (12): 1151-62).

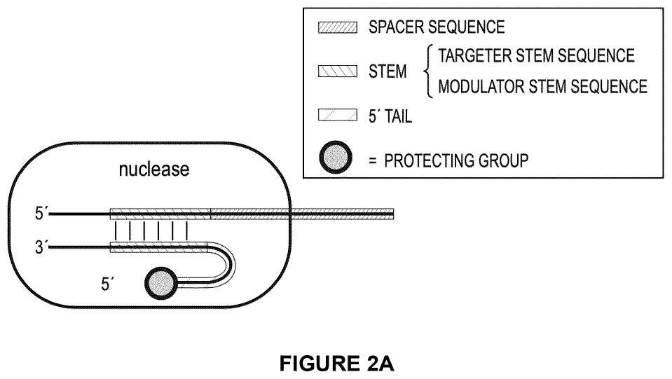

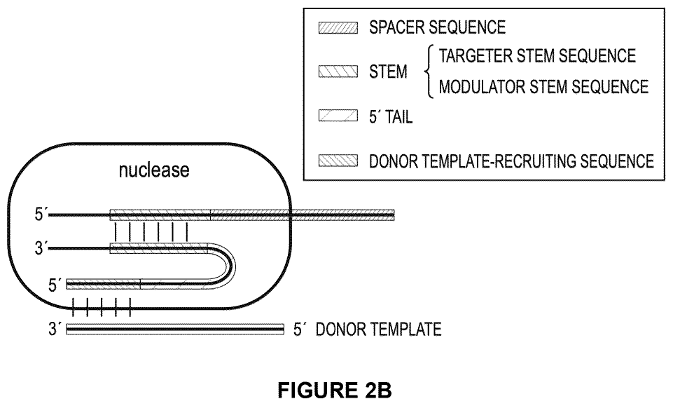

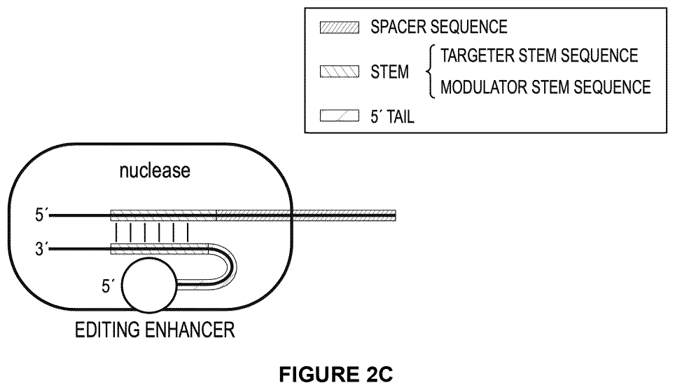

The targeter nucleic acid is directed to a specific target nucleotide sequence, and a donor template can be designed to modify the target nucleotide sequence or a sequence nearby. It is understood, therefore, that association of the single guide nucleic acid, the targeter nucleic acid, or the modulator nucleic acid with a donor template can increase editing efficiency and reduce off-targeting. Accordingly, in certain embodiments, the single guide nucleic acid or the modulator nucleic acid further comprises a donor template-recruiting sequence capable of hybridizing with a donor template (see FIG. 2B). Donor templates are described in the “Donor Templates” subsection of section II infra. The donor template and donor template-recruiting sequence can be designed such that they bear sequence complementarity. In certain embodiments, the donor template-recruiting sequence is at least 90% (e.g., at least 91%, at least 92%, at least 93%, at least 94%, at least 95%, at least 96%, at least 97%, at least 98%, or at least 99%) complementary to at least a portion of the donor template. In certain embodiments, the donor template-recruiting sequence is 100% complementary to at least a portion of the donor template. In certain embodiments, where the donor template comprises an engineered sequence not homologous to the sequence to be repaired, the donor template-recruiting sequence is capable of hybridizing with the engineered sequence in the donor template. In certain embodiments, the donor template-recruiting sequence is at least 20, 25, 30, 35, 40, 45, 50, 55, 60, 65, 70, 75, 80, 85, 90, 95, or 100 nucleotides in length. In certain embodiments, the donor template-recruiting sequence is positioned at or near the 5′ end of the single guide nucleic acid or at or near the 5′ end of the modulator nucleic acid. In certain embodiments, the donor template-recruiting sequence is linked to the 5′ tail, if present, or to the modulator stem sequence, of the single guide nucleic acid or the modulator nucleic acid through an internucleotide bond or a nucleotide linker.

In certain embodiments, the single guide nucleic acid or the modulator nucleic acid further comprises an editing enhancer sequence, which increases the efficiency of gene editing and/or homology-directed repair (HDR) (see FIG. 2C). Exemplary editing enhancer sequences are described in Park et al. (2018) NAT. COMMUN. 9:3313. In certain embodiments, the editing enhancer sequence is positioned 5′ to the 5′ tail, if present, or 5′ to the single guide nucleic acid or the modulator stem sequence. In certain embodiments, the editing enhancer sequence is 1-50, 4-50, 9-50, 15-50, 25-50, 1-25, 4-25, 9-25, 15-25, 1-15, 4-15, 9-15, 1-9, 4-9, or 1-4 nucleotides in length. In certain embodiments, the editing enhancer sequence is about 1, 2, 3, 4, 5, 6, 7, 8, 9, 10, 15, 20, 25, 30, 35, 40, 45, 50, or 55 nucleotides in length. The editing enhancer sequence is designed to minimize homology to the target nucleotide sequence or any other sequence that the engineered, non-naturally occurring system may be contacted to, e.g., the genome sequence of a cell into which the engineered, non-naturally occurring system is delivered. In certain embodiments, the editing enhancer is designed to minimize the presence of hairpin structure. The editing enhancer can comprise one or more of the chemical modifications disclosed herein.

The single guide nucleic acid, the modulator nucleic acid, and/or the targeter nucleic acid can further comprise a protective nucleotide sequence that prevents or reduces nucleic acid degradation. In certain embodiments, the protective nucleotide sequence is at least 5 (e.g., at least 10, at least 15, at least 20, at least 25, at least 30, at least 35, at least 40, at least 45, or at least 50) nucleotides in length. The length of the protective nucleotide sequence increases the time for an exonuclease to reach the 5′ tail, modulator stem sequence, targeter stem sequence, and/or spacer sequence, thereby protecting these portions of the single guide nucleic acid, the modulator nucleic acid, and/or the targeter nucleic acid from degradation by an exonuclease. In certain embodiments, the protective nucleotide sequence forms a secondary structure, such as a hairpin or a tRNA structure, to reduce the speed of degradation by an exonuclease (see, for example, Wu et al. (2018) CELL. MOL. LIFE SCI., 75 (19): 3593-3607). Secondary structures can be predicted by methods known in the art, such as the online webserver RNAfold developed at University of Vienna using the centroid structure prediction algorithm (see, Gruber et al. (2008) NUCLEIC ACIDS RES., 36: W70). Certain chemical modifications, which may be present in the protective nucleotide sequence, can also prevent or reduce nucleic acid degradation, as disclosed in the “RNA Modifications” subsection infra.

A protective nucleotide sequence is typically located at the 5′ or 3′ end of the single guide nucleic acid, the modulator nucleic acid, and/or the targeter nucleic acid. In certain embodiments, the single guide nucleic acid comprises a protective nucleotide sequence at the 5′ end, at the 3′ end, or at both ends, optionally through a nucleotide linker. In certain embodiments, the modulator nucleic acid comprises a protective nucleotide sequence at the 5′ end, at the 3′ end, or at both ends, optionally through a nucleotide linker. In particular embodiments, the modulator nucleic acid comprises a protective nucleotide sequence at the 5′ end (see FIG. 2A). In certain embodiments, the targeter nucleic acid comprises a protective nucleotide sequence at the 5′ end, at the 3′ end, or at both ends, optionally through a nucleotide linker.

As described above, various nucleotide sequences can be present in the 5′ portion of a single nucleic acid or a modulator nucleic acid, including but not limited to a donor template-recruiting sequence, an editing enhancer sequence, a protective nucleotide sequence, and a linker connecting such sequence to the 5′ tail, if present, or to the modulator stem sequence. It is understood that the functions of donor template recruitment, editing enhancement, protection against degradation, and linkage are not exclusive to each other, and one nucleotide sequence can have one or more of such functions. For example, in certain embodiments, the single guide nucleic acid or the modulator nucleic acid comprises a nucleotide sequence that is both a donor template-recruiting sequence and an editing enhancer sequence. In certain embodiments, the single guide nucleic acid or the modulator nucleic acid comprises a nucleotide sequence that is both a donor template-recruiting sequence and a protective sequence. In certain embodiments, the single guide nucleic acid or the modulator nucleic acid comprises a nucleotide sequence that is both an editing enhancer sequence and a protective sequence. In certain embodiments, the single guide nucleic acid or the modulator nucleic acid comprises a nucleotide sequence that is a donor template-recruiting sequence, an editing enhancer sequence, and a protective sequence. In certain embodiments, the nucleotide sequence 5′ to the 5′ tail, if present, or 5′ to the modulator stem sequence is 1-90, 1-80, 1-70, 1-60, 1-50, 1-40, 1-30, 1-20, 1-10, 10-90, 10-80, 10-70, 10-60, 10-50, 10-40, 10-30, 10-20, 20-90, 20-80, 20-70, 20-60, 20-50, 20-40, 20-30, 30-90, 30-80, 30-70, 30-60, 30-50, 30-40, 40-90, 40-80, 40-70, 40-60, 40-50, 50-90, 50-80, 50-70, 50-60, 60-90, 60-80, 60-70, 70-90, 70-80, or 80-90 nucleotides in length.

In certain embodiments, an engineered, non-naturally occurring system further comprises one or more compounds (e.g., small molecule compounds) that enhance HDR and/or inhibit NHEJ. Exemplary compounds having such functions are described in Maruyama et al. (2015) NAT BIOTECHNOL. 33 (5): 538-42; Chu et al. (2015) NAT BIOTECHNOL. 33 (5): 543-48; Yu et al. (2015) CELL STEM CELL 16 (2): 142-47; Pinder et al. (2015) NUCLEIC ACIDS RES. 43 (19): 9379-92; and Yagiz et al. (2019) COMMUN. BIOL. 2:198. In certain embodiments, an engineered, non-naturally occurring system further comprises one or more compounds selected from the group consisting of DNA ligase IV antagonists (e.g., SCR7 compound, Ad4 E1B55K protein, and Ad4 E4orf6 protein), RAD51 agonists (e.g., RS-1), DNA-dependent protein kinase (DNA-PK) antagonists (e.g., NU7441 and KU0060648), β3-adrenergic receptor agonists (e.g., L755507), inhibitors of intracellular protein transport from the ER to the Golgi apparatus (e.g., brefeldin A), and any combinations thereof.

In certain embodiments, an engineered, non-naturally occurring system comprising a targeter nucleic acid and a modulator nucleic acid is tunable or inducible. For example, in certain embodiments, the targeter nucleic acid, the modulator nucleic acid, and/or the Cas protein can be introduced to the target nucleotide sequence at different times, the system becoming active only when all components are present. In certain embodiments, the amounts of the targeter nucleic acid, the modulator nucleic acid, and/or the Cas protein can be titrated to achieve desired efficiency and specificity. In certain embodiments, excess amount of a nucleic acid comprising the targeter stem sequence or the modulator stem sequence can be added to the system, thereby dissociating the complex of the targeter nucleic and modulator nucleic acid and turning off the system.

### C. gNA Modifications

Guide nucleic acids, including a single guide nucleic acid, a targeter nucleic acid, and/or a modulator nucleic acid, may comprise a DNA (e.g., modified DNA), an RNA (e.g., modified RNA), or a combination thereof. In certain embodiments, the single guide nucleic acid comprises a DNA (e.g., modified DNA), an RNA (e.g., modified RNA), or a combination thereof. In certain embodiments, the targeter nucleic acid comprises a DNA (e.g., modified DNA), an RNA (e.g., modified RNA), or a combination thereof. In certain embodiments, the modulator nucleic acid comprises a DNA (e.g., modified DNA), an RNA (e.g., modified RNA), or a combination thereof. Spacer sequences can be presented as DNA sequences by including thymidines (T) rather than uridines (U). It is understood that corresponding RNA sequences and DNA/RNA chimeric sequences are also contemplated. For example, where the spacer sequence is an RNA, its sequence can be derived from a DNA sequence disclosed herein by replacing each T with U. As a result, for the purpose of describing a nucleotide sequence, T and U are used interchangeably herein.

In certain embodiments engineered, non-naturally occurring systems comprising a targeter nucleic acid comprising: a spacer sequence designed to hybridize with a target nucleotide sequence and a targeter stem sequence; and a modulator nucleic acid comprising a modulator stem sequence complementary to the targeter stem sequence, and, optionally, a 5′ sequence, e.g., a tail sequence, wherein, in a single guide nucleic acid the targeter nucleic acid and the modulator nucleic acid are part of a single polynucleotide, and in a dual guide nucleic acid, the targeter nucleic acid and the modulator nucleic acid are separate nucleic acids; modifications can include one or more chemical modifications to one or more nucleotides or internucleotide linkages at or near the 3′ end of the targeter nucleic acid (dual and single gNA), at or near the 5′ end of the targeter nucleic acid (dual gNA), at or near the 3′ end of the modulator nucleic acid (dual gNA), at or near the 5′ end of the modulator nucleic acid (single and dual gNA), or combinations thereof as appropriate for single or dual gNA. In certain embodiments, the Cas nuclease is a type V-A Cas nuclease. Modulator and/or targeter nucleic sequences can include further sequences, as detailed in the Guide Nucleic Acids section, and modifications can be in these further sequences, as appropriate and apparent to one of skill in the art. In embodiments described in this section, below, in certain embodiments, guide nucleic acid is oriented from 5′ at the modulator nucleic acid to 3′ at the modulator stem sequence, and 5′ at the targeter stem sequence to 3′ at the targeter sequence (see, e.g., FIGS. 1A and 1B); in certain embodiments, as appropriate, guide nucleic acid is oriented from 3′ at the modulator nucleic acid to 5′ at the modulator stem sequence, and 3′ at the targeter stem sequence to 5′ at the targeter sequence.

The targeter nucleic acid may comprise a DNA (e.g., modified DNA), an RNA (e.g., modified RNA), or a combination thereof. The modulator nucleic acid may comprise a DNA (e.g., modified DNA), an RNA (e.g., modified RNA), or a combination thereof. In certain embodiments, the targeter nucleic acid is an RNA and the modulator nucleic acid is an RNA. A targeter nucleic acid in the form of an RNA is also called targeter RNA, and a modulator nucleic acid in the form of an RNA is also called modulator RNA. The nucleotide sequences disclosed herein are presented as DNA sequences by including thymidines (T) and/or RNA sequences including uridines (U). It is understood that corresponding DNA sequences, RNA sequences, and DNA/RNA chimeric sequences are also contemplated. For example, where a spacer sequence is presented as a DNA sequence, a nucleic acid comprising this spacer sequence as an RNA can be derived from the DNA sequence disclosed herein by replacing each T with U. As a result, for the purpose of describing a nucleotide sequence, T and U are used interchangeably herein.

In certain embodiments some or all of the gNA is RNA, e.g., a gRNA. In certain embodiments, 5-100%, 10-100%, 20-100%, 30-100%, 40-100%, 50-100%, 60-100%, 70-100%, 80-100%, 90-100%, 95-100%, 99-100%, 99.5-100% of the gNA is gRNA. In certain embodiments, 20%-80%, 20%-70%, 20%-60%, 20%-50%, 20%-40%, 20%-30%, 30%-80%, 30%-70%, 30%-60%, 30%-50%, 30%-40%, 40%-80%, 40%-70%, 40%-60%, 40%-50%, 50%-80%, 50%-70%, 50%-60%, 60%-80%, 60%-70%, or 70%-80% of gNA is RNA. In certain embodiments, 50% of the gNA is RNA. In certain embodiments, 70% of the gNA is RNA. In certain embodiments, 90% of the gNA is RNA. In certain embodiments, 100% of the gNA is RNA, e.g., a gRNA. In further embodiments, the remaining portion of the gNA that is not RNA comprises a modified ribonucleotide, a deoxyribonucleotide, a modified deoxyribonucleotide, or a synthetic, e.g., unnatural nucleotide, for example, not intended to be limiting, threose nucleic acid, locked nucleic acid, peptide nucleic acid, arabinonucleic acid, hexose nucleic acid, among others.

In certain embodiments, the targeter nucleic acid and/or the modulator nucleic acid are RNAs with one or more modifications in a ribose group, one or more modifications in a phosphate group, one or more modifications in a nucleobase, one or more terminal modifications, or a combination thereof. Exemplary modifications are disclosed in U.S. Pat. Nos. 10,900,034 and 10,767,175, U.S. Patent Application Publication No. 2018/0119140, Watts et al. (2008) DRUG DISCOV. TODAY 13:842-55, and Hendel et al. (2015) NAT. BIOTECHNOL. 33:985.

In certain embodiments, a targeter nucleic acid, e.g., RNA, comprises at least one nucleotide at or near the 3′ end comprising a modification to a ribose, phosphate group, nucleobase, or terminal modification. In certain embodiments, the 3′ end of the targeter nucleic acid comprises the spacer sequence. In certain embodiments, the 3′ end of the targeter nucleic acid comprises the targeter stem sequence. Exemplary modifications are disclosed in Dang et al. (2015) GENOME BIOL. 16:280, Kocaz et al. (2019) NATURE BIOTECH. 37:657-66, Liu et al. (2019) NUCLEIC ACIDS RES. 47 (8): 4169-4180, Schubert et al. (2018) J. CYTOKINE BIOL. 3 (1): 121, Teng et al. (2019) GENOME BIOL. 20 (1): 15, Watts et al. (2008) DRUG DISCOV. TODAY 13 (19-20): 842-55, and Wu et al. (2018) CELL MOL. LIFE. SCI. 75 (19): 3593-607.

Modifications in a ribose group include but are not limited to modifications at the 2′ position or modifications at the 4′ position. For example, in certain embodiments, the ribose comprises 2′-O—C1-4alkyl, such as 2′-O-methyl (2′-OMe, or M). In certain embodiments, the ribose comprises 2′-O—C1-3alkyl-O—C1-3alkyl, such as 2′-methoxyethoxy (2′-O—CH₂CH₂OCH₃) also known as 2′-O-(2-methoxyethyl) or 2′-MOE. In certain embodiments, the ribose comprises 2′-O-allyl. In certain embodiments, the ribose comprises 2′-O-2,4-Dinitrophenol (DNP). In certain embodiments, the ribose comprises 2′-halo, such as 2′-F, 2′-Br, 2′-Cl, or 2′-I. In certain embodiments, the ribose comprises 2′—NH₂. In certain embodiments, the ribose comprises 2′-H (e.g., a deoxynucleotide). In certain embodiments, the ribose comprises 2′-arabino or 2′-F-arabino. In certain embodiments, the ribose comprises 2′-LNA or 2′-ULNA. In certain embodiments, the ribose comprises a 4′-thioribosyl.

Modifications can also include a deoxy group, for example a 2′-deoxy-3′-phosphonoacetate (DP), a 2′-deoxy-3′-thiophosphonoacetate (DSP).

Internucleotide linkage modifications in a phosphate group include but are not limited to a phosphorothioate(S), a chiral phosphorothioate, a phosphorodithioate, a boranophosphonate, a C₁₋₄alkyl phosphonate such as a methylphosphonate, a boranophosphonate, a phosphonocarboxylate such as a phosphonoacetate (P), a phosphonocarboxylate ester such as a phosphonoacetate ester, an amide, a thiophosphonocarboxylate such as a thiophosphonoacetate (SP), a thiophosphonocarboxylate ester such as a thiophosphonoacetate ester, and a 2′,5′-linkage having a phosphodiester or any of the modified phosphates above. Various salts, mixed salts and free acid forms are also included.

Modifications in a nucleobase include but are not limited to 2-thiouracil, 2-thiocytosine, 4-thiouracil, 6-thioguanine, 2-aminoadenine, 2-aminopurine, pseudouracil, hypoxanthine, 7-deazaguanine, 7-deaza-8-azaguanine, 7-deazaadenine, 7-deaza-8-azaadenine, 5-methylcytosine, 5-methyluracil, 5-hydroxymethylcytosine, 5-hydroxymethyluracil, 5,6-dehydrouracil, 5-propynylcytosine, 5-propynyluracil, 5-ethynylcytosine, 5-cthynyluracil, 5-allyluracil, 5-allylcytosine, 5-aminoallyluracil, 5-aminoallyl-cytosine, 5-bromouracil, 5-iodouracil, diaminopurine, difluorotoluene, dihydrouracil, an abasic nucleotide, Z base, P base, Unstructured Nucleic Acid, isoguanine, isocytosine (see, Piccirilli et al. (1990) NATURE, 343:33), 5-methyl-2-pyrimidine (see, Rappaport (1993) BIOCHEMISTRY, 32:3047), x (A,G,C,T), and y (A,G,C,T).

Terminal modifications include but are not limited to polyethyleneglycol (PEG), hydrocarbon linkers (such as heteroatom (O,S,N)-substituted hydrocarbon spacers; halo-substituted hydrocarbon spacers; keto-, carboxyl-, amido-, thionyl-, carbamoyl-, thionocarbamaoyl-containing hydrocarbon spacers, propanediol), spermine linkers, dyes such as fluorescent dyes (for example, fluoresceins, rhodamines, cyanines), quenchers (for example, dabcyl, BHQ), and other labels (for example biotin, digoxigenin, acridine, streptavidin, avidin, peptides and/or proteins). In certain embodiments, a terminal modification comprises a conjugation (or ligation) of the RNA to another molecule comprising an oligonucleotide (such as deoxyribonucleotides and/or ribonucleotides), a peptide, a protein, a sugar, an oligosaccharide, a steroid, a lipid, a folic acid, a vitamin and/or other molecule. In certain embodiments, a terminal modification incorporated into the RNA is located internally in the RNA sequence via a linker such as 2-(4-butylamidofluorescein) propane-1,3-diol bis (phosphodiester) linker, which is incorporated as a phosphodiester linkage and can be incorporated anywhere between two nucleotides in the RNA.

The modifications disclosed above can be combined in the targeter nucleic acid and/or the modulator nucleic acid that are in the form of RNA. In certain embodiments, the modification in the RNA is selected from the group consisting of incorporation of 2′-O-methyl-3′phosphorothioate (MS), 2′-O-methyl-3′-phosphonoacetate (MP), 2′-O-methyl-3′-thiophosphonoacetate (MSP), 2′-halo-3′-phosphorothioate (e.g., 2′-fluoro-3′-phosphorothioate), 2′-halo-3′-phosphonoacetate (e.g., 2′-fluoro-3′-phosphonoacetate), and 2′-halo-3′-thiophosphonoacetate (e.g., 2′-fluoro-3′-thiophosphonoacetate).

In certain embodiments, modifications can include 2′-O-methyl (M), a phosphorothioate(S), a phosphonoacetate (P), a thiophosphonoacetate (SP), a 2′-O-methyl-3′-phosphorothioate (MS), a 2′-O-methyl-3′-phosphonoacetate (MP), a 2′-O-methyl-3′-thiophosphonoacetate (MSP), a 2′-deoxy-3′-phosphonoacetate (DP), a 2′-deoxy-3′-thiophosphonoacetate (DSP), or a combination thereof, at or near either the 3′ or 5′ end of either the targeter or modulator nucleic acid, as appropriate for single or dual gNA. In certain embodiments, modifications can include either a 5′ or a 3′ propanediol or C3 linker modification.

In certain embodiments, the modification alters the stability of the RNA. In certain embodiments, the modification enhances the stability of the RNA, e.g., by increasing nuclease resistance of the RNA relative to a corresponding RNA without the modification. Stability-enhancing modifications include but are not limited to incorporation of 2′-O-methyl, a 2′-O—C₁₋₄alkyl, 2′-halo (e.g., 2′-F, 2′-Br, 2′-Cl, or 2′-I), 2′MOE, a 2′-O—C1-3alkyl-O—C1-3alkyl, 2′—NH₂, 2′-H (or 2′-deoxy), 2′-arabino, 2′-F-arabino, 4′-thioribosyl sugar moiety, 3′-phosphorothioate, 3′-phosphonoacetate, 3′-thiophosphonoacetate, 3′-methylphosphonate, 3′-boranophosphate, 3′-phosphorodithioate, locked nucleic acid (“LNA”) nucleotide which comprises a methylene bridge between the 2′ and 4′ carbons of the ribose ring, and unlocked nucleic acid (“ULNA”) nucleotide. Such modifications are suitable for use as a protecting group to prevent or reduce degradation of the 5′ sequence, e.g., a tail sequence, modulator stem sequence (dual guide nucleic acids), targeter stem sequence (dual guide nucleic acids), and/or spacer sequence (see, the “Targeter and Modulator nucleic acids” subsection).

In certain embodiments, the modification alters the specificity of the engineered, non-naturally occurring system. In certain embodiments, the modification enhances the specificity of the engineered, non-naturally occurring system, e.g., by enhancing on-target binding and/or cleavage, or reducing off-target binding and/or cleavage, or a combination thereof. Specificity-enhancing modifications include but are not limited to 2-thiouracil, 2-thiocytosine, 4-thiouracil, 6-thioguanine, 2-aminoadenine, and pseudouracil. Within 10, 5, 4, 3, 2, or 1 nucleotide of the 3′ end, for example the 3′ end nucleotide, is modified.

In certain embodiments, the modification alters the immunostimulatory effect of the RNA relative to a corresponding RNA without the modification. For example, in certain embodiments, the modification reduces the ability of the RNA to activate TLR7, TLR8, TLR9, TLR3, RIG-I, and/or MDA5.

In certain embodiments, the targeter nucleic acid and/or the modulator nucleic acid comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 modified nucleotides or internucleotide linkages. The modification can be made at one or more positions in the targeter nucleic acid and/or the modulator nucleic acid such that these nucleic acids retain functionality. For example, the modified nucleic acids can still direct the Cas protein to the target nucleotide sequence and allow the Cas protein to exert its effector function. It is understood that the particular modification(s) at a position may be selected based on the functionality of the nucleotide or internucleotide linkage at the position. For example, a specificity-enhancing modification may be suitable for a nucleotide or internucleotide linkage in the spacer sequence, the targeter stem sequence, or the modulator stem sequence. A stability-enhancing modification may be suitable for one or more terminal nucleotides or internucleotide linkages in the targeter nucleic acid and/or the modulator nucleic acid. In certain embodiments, at least 1 (e.g., at least 2, at least 3, at least 4, or at least 5) terminal nucleotides or internucleotide linkages at or near the 5′ end and/or at least 1 (e.g., at least 2, at least 3, at least 4, or at least 5) terminal nucleotides or internucleotide linkages at or near the 3′ end of the targeter nucleic acid are modified. In certain embodiments, 5 or fewer (e.g., 1 or fewer, 2 or fewer, 3 or fewer, or 4 or fewer) terminal nucleotides or internucleotide linkages at or near the 5′ end and/or 5 or fewer (e.g., 1 or fewer, 2 or fewer, 3 or fewer, or 4 or fewer) terminal nucleotides or internucleotide linkages at or near the 3′ end of the targeter nucleic acid are modified. In certain embodiments, at least 1 (e.g., at least 2, at least 3, at least 4, or at least 5) terminal nucleotides or internucleotide linkages at or near the 5′ end and/or at least 1 (e.g., at least 2, at least 3, at least 4, or at least 5) terminal nucleotides or internucleotide linkages at or near the 3′ end of the modulator nucleic acid are modified. In certain embodiments, 5 or fewer (e.g., 1 or fewer, 2 or fewer, 3 or fewer, or 4 or fewer) terminal nucleotides or internucleotide linkages at or near the 5′ end and/or 5 or fewer (e.g., 1 or fewer, 2 or fewer, 3 or fewer, or 4 or fewer) terminal nucleotides or internucleotide linkages at or near the 3′ end of the modulator nucleic acid are modified. Selection of positions for modifications is described in U.S. Pat. Nos. 10,900,034 and 10,767,175. As used in this paragraph, where the targeter or modulator nucleic acid is a combination of DNA and RNA, the nucleic acid as a whole is considered as an RNA, and the DNA nucleotide(s) are considered as modification(s) of the RNA, including a 2′-H modification of the ribose and optionally a modification of the nucleobase.

It is understood that, in dual guide nucleic acid systems the targeter nucleic acid and the modulator nucleic acid, while not in the same nucleic acids, i.e., not linked end-to-end through a traditional internucleotide bond, can be covalently conjugated to each other through one or more chemical modifications introduced into these nucleic acids, thereby increasing the stability of the double-stranded complex and/or improving other characteristics of the system.

### III. COMPOSITION AND METHODS FOR TARGETING, EDITING, AND/OR MODIFYING GENOMIC DNA

An engineered, non-naturally occurring system, such as disclosed herein, can be useful for targeting, editing, and/or modifying a target nucleic acid, such as a DNA (e.g., genomic DNA) in a cell or organism.

The present invention provides a method of cleaving a target nucleic acid (e.g., DNA) comprising the sequence of a preselected target sequence or a portion thereof, the method comprising contacting the target DNA with an engineered, non-naturally occurring system disclosed herein, thereby resulting in cleavage of the target DNA.

In addition, the present invention provides a method of binding a target nucleic acid (e.g., DNA) comprising the sequence of a preselected target sequence or a portion thereof, the method comprising contacting the target DNA with an engineered, non-naturally occurring system disclosed herein, thereby resulting in binding of the system to the target DNA. This method can be useful, e.g., for detecting the presence and/or location of a preselected target gene, for example, if a component of the system (e.g., the Cas protein) comprises a detectable marker.

In addition, provided are methods of modifying a target nucleic acid (e.g., DNA) comprising the sequence of a preselected target sequence or a portion thereof, or a structure (e.g., protein) associated with the target DNA (e.g., a histone protein in a chromosome), the method comprising contacting the target DNA with an engineered, non-naturally occurring system disclosed herein, wherein the Cas protein comprises an effector domain or is associated with an effector protein, thereby resulting in modification of the target DNA or the structure associated with the target DNA. The modification corresponds to the function of the effector domain or effector protein. Exemplary functions described in the “Cas Proteins” subsection in Section I supra are applicable hereto.

An engineered, non-naturally occurring system can be contacted with the target nucleic acid as a complex. Accordingly, in certain embodiments, a method comprises contacting the target nucleic acid with a CRISPR-Cas complex comprising a targeter nucleic acid, a modulator nucleic acid, and a Cas protein disclosed herein. In certain embodiments, the Cas protein is a type V-A, type V-C, or type V-D Cas protein (e.g., Cas nuclease). In certain embodiments, the Cas protein is a type V-A Cas protein (e.g., Cas nuclease).

In certain embodiments, provided is a method of editing a human genomic sequence at one of a group of preselected target gene loci, the method comprising delivering an engineered, non-naturally occurring system disclosed herein into a human cell, thereby resulting in editing of the genomic sequence at the target gene locus in the human cell. In certain embodiments, provided herein is a method of detecting a human genomic sequence at one of a group of preselected target gene loci, the method comprising delivering the engineered, non-naturally occurring system disclosed herein into a human cell, wherein a component of the system (e.g., the Cas protein) comprises a detectable marker, thereby detecting the target gene locus in the human cell. In certain embodiments, provided herein is a method of modifying a human chromosome at one of a group of preselected target gene loci, the method comprising delivering the engineered, non-naturally occurring system disclosed herein into a human cell, wherein the Cas protein comprises an effector domain or is associated with an effector protein, thereby resulting in modification of the chromosome at the target gene locus in the human cell.

The CRISPR-Cas complex may be delivered to a cell by introducing a pre-formed ribonucleoprotein (RNP) complex into the cell. Alternatively, one or more components of the CRISPR-Cas complex may be expressed in the cell. Exemplary methods of delivery are known in the art and described in, for example, U.S. Pat. Nos. 8,697,359, 10,113,167, 10,570,418, 10,829,787, 11,118,194, and 11,125,739 and U.S. Patent Application Publication Nos.

2015/0344912, 2018/0119140, and 2018/0282763.

It is understood that contacting a DNA (e.g., genomic DNA) in a cell with a CRISPR-Cas complex does not require delivery of all components of the complex into the cell. For example, one or more of the components may be pre-existing in the cell. In certain embodiments, the cell (or a parental/ancestral cell thereof) has been engineered to express the Cas protein, and the single guide nucleic acid (or a nucleic acid comprising a regulatory element operably linked to a nucleotide sequence encoding the single guide nucleic acid), the targeter nucleic acid (or a nucleic acid comprising a regulatory element operably linked to a nucleotide sequence encoding the targeter nucleic acid), and/or the modulator nucleic acid (or a nucleic acid comprising a regulatory element operably linked to a nucleotide sequence encoding the modulator nucleic acid) are delivered into the cell. In certain embodiments, the cell (or a parental/ancestral cell thereof) has been engineered to express the modulator nucleic acid, and the Cas protein (or a nucleic acid comprising a regulatory element operably linked to a nucleotide sequence encoding the Cas protein) and the targeter nucleic acid (or a nucleic acid comprising a regulatory element operably linked to a nucleotide sequence encoding the targeter nucleic acid) are delivered into the cell. In certain embodiments, the cell (or a parental/ancestral cell thereof) has been engineered to express the Cas protein and the modulator nucleic acid, and the targeter nucleic acid (or a nucleic acid comprising a regulatory element operably linked to a nucleotide sequence encoding the targeter nucleic acid) is delivered into the cell.

In certain embodiments, the target DNA is in the genome of a target cell. Accordingly, the present invention also provides a cell comprising the non-naturally occurring system or a CRISPR expression system described herein. In addition, the present invention provides a cell whose genome has been modified by the CRISPR-Cas system or complex disclosed herein.

The target cells can be mitotic or post-mitotic cells from any organism, such as a bacterial cell (e.g., E coli), an archacal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. Agardh, or the like, a fungal cell (e.g., a yeast cell, such as S. cervisiae), an animal cell, a cell from an invertebrate animal (e.g. fruit fly, enidarian, echinoderm, nematode, etc.), a cell from a vertebrate animal (e.g., fish, amphibian, reptile, bird, mammal), a cell from a mammal, a cell from a rodent, or a cell from a human. The types of target cells include but are not limited to a stem cell (e.g., an embryonic stem (ES) cell, an induced pluripotent stem (iPS) cell, a germ cell), a somatic cell (e.g., a fibroblast, a hematopoietic cell, a T lymphocyte (e.g., CD8+T lymphocyte), an NK cell, a neuron, a muscle cell, a bone cell, a hepatocyte, a pancreatic cell), an in vitro or in vivo embryonic cell of an embryo at any stage (e.g., a 1-cell, 2-cell, 4-cell, 8-cell; stage zebrafish embryo). Cells may be from established cell lines or may be primary cells (i.e., cells and cells cultures that have been derived from a subject and allowed to grow in vitro for a limited number of passages of the culture). For example, primary cultures are cultures that may have been passaged within 0 times, 1 time, 2 times, 4 times, 5 times, 10 times, or 15 times, but not enough times to go through the crisis stage. Typically, the primary cell lines are maintained for fewer than 10 passages in vitro. If the cells are primary cells, they may be harvest from an individual by any suitable method. For example, leukocytes may be harvested by apheresis, leukocytapheresis, or density gradient separation, while cells from tissues such as skin, muscle, bone marrow, spleen, liver, pancreas, lung, intestine, or stomach can be harvested by biopsy. The harvested cells may be used immediately, or may be stored under frozen conditions with a cryopreservative and thawed at a later time in a manner as commonly known in the art.

### A. Ribonucleoprotein (RNP) Delivery and “Cas RNA” Delivery

An engineered, non-naturally occurring system disclosed herein can be delivered into a cell by suitable methods known in the art, including but not limited to ribonucleoprotein (RNP) delivery and “Cas RNA” delivery described below.

In certain embodiments, a CRISPR-Cas system including a single guide nucleic acid and a Cas protein, or a CRISPR-Cas system including a targeter nucleic acid, a modulator nucleic acid, and a Cas protein, can be combined into a RNP complex and then delivered into the cell as a pre-formed complex. This method is suitable for active modification of the genetic or epigenetic information in a cell during a limited time period. For example, where the Cas protein has nuclease activity to modify the genomic DNA of the cell, the nuclease activity only needs to be retained for a period of time to allow DNA cleavage, and prolonged nuclease activity may increase off-targeting. Similarly, certain epigenetic modifications can be maintained in a cell once established and can be inherited by daughter cells.

A “ribonucleoprotein” or “RNP,” as used herein, can refer to a complex comprising a nucleoprotein and a ribonucleic acid. A “nucleoprotein” as provided herein can refer to a protein capable of binding a nucleic acid (e.g., RNA, DNA). Where the nucleoprotein binds a ribonucleic acid it can be referred to as “ribonucleoprotein.” The interaction between the ribonucleoprotein and the ribonucleic acid may be direct, e.g., by covalent bond, or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions, or the like). In certain embodiments, the ribonucleoprotein includes an RNA-binding motif non-covalently bound to the ribonucleic acid. For example, positively charged aromatic amino acid residues (e.g., lysine residues) in the RNA-binding motif may form electrostatic interactions with the negative nucleic acid phosphate backbones of the RNA.

To ensure efficient loading of the Cas protein, the single guide nucleic acid, or the combination of the targeter nucleic acid and the modulator nucleic acid, can be provided in excess molar amount (e.g., at least 2 fold, at least 3 fold, at least 4 fold, or at least 5 fold) relative to the Cas protein. In certain embodiments, the targeter nucleic acid and the modulator nucleic acid are annealed under suitable conditions prior to complexing with the Cas protein. In other embodiments, the targeter nucleic acid, the modulator nucleic acid, and the Cas protein are directly mixed together to form an RNP.

A variety of delivery methods can be used to introduce an RNP disclosed herein into a cell. Exemplary delivery methods or vehicles include but are not limited to microinjection, liposomes (see, e.g., U.S. Pat. No. 10,829,787,) such as molecular trojan horses liposomes that delivers molecules across the blood brain barrier (see, Pardridge et al. (2010) COLD SPRING HARB. PROTOC., doi: 10.1101/pdb.prot5407), immunoliposomes, virosomes, microvesicles (e.g., exosomes and ARMMs), polycations, lipid: nucleic acid conjugates, electroporation, cell permeable peptides (see, U.S. Pat. No. 11,118,194), nanoparticles, nanowires (see, Shalek et al. (2012) NANO LETTERS, 12:6498), exosomes, and perturbation of cell membrane (e.g., by passing cells through a constriction in a microfluidic system, see, U.S. Pat. No. 11,125,739). Where the target cell is a proliferating cell, the efficiency of RNP delivery can be enhanced by cell cycle synchronization (see, U.S. Pat. No. 10,570,418). In certain embodiments, an RNP is delivered into a cell by electroporation.

In certain embodiments, a CRISPR-Cas system is delivered into a cell in a “approach, i.e., delivering (a) a single guide nucleic acid, or a combination of a targeter nucleic acid and a modulator nucleic acid, and (b) an RNA (e.g., messenger RNA (mRNA)) encoding a Cas protein. The RNA encoding the Cas protein can be translated in the cell and form a complex with the single guide nucleic acid or combination of the targeter nucleic acid and the modulator nucleic acid intracellularly. Similar to the RNP approach, RNAs have limited half-lives in cells, even though stability-increasing modification(s) can be made in one or more of the RNAs.

Accordingly, the “Cas RNA” approach is suitable for active modification of the genetic or epigenetic information in a cell during a limited time period, such as DNA cleavage, and has the advantage of reducing off-targeting.

The mRNA can be produced by transcription of a DNA comprising a regulatory element operably linked to a Cas coding sequence. Given that multiple copies of Cas protein can be generated from one mRNA, the single guide nucleic acid, or the targeter nucleic acid and the modulator nucleic acid are generally provided in excess molar amount (e.g., at least 5 fold, at least 10 fold, at least 20 fold, at least 30 fold, at least 50 fold, or at least 100 fold) relative to the mRNA. In certain embodiments, the targeter nucleic acid and the modulator nucleic acid are annealed under suitable conditions prior to delivery into the cells. In other embodiments, the targeter nucleic acid and the modulator nucleic acid are delivered into the cells without annealing in vitro.

A variety of delivery systems can be used to introduce an “Cas RNA” system into a cell. Non-limiting examples of delivery methods or vehicles include microinjection, biolistic particles, liposomes (see, e.g., U.S. Pat. No. 10,829,787) such as molecular trojan horses liposomes that delivers molecules across the blood brain barrier (see, Pardridge et al. (2010) COLD SPRING HARB. PROTOC., doi: 10.1101/pdb.prot5407), immunoliposomes, virosomes, polycations, lipid: nucleic acid conjugates, electroporation, nanoparticles, nanowires (see, Shalek et al. (2012) NANO LETTERS, 12:6498), exosomes, and perturbation of cell membrane (e.g., by passing cells through a constriction in a microfluidic system, see, U.S. Pat. No. 11,125,739). Specific examples of the “nucleic acid only” approach by electroporation are described in International (PCT) Publication No. WO 2016/164356.

In certain embodiments, the CRISPR-Cas system is delivered into a cell in the form of (a) a single guide nucleic acid or a combination of a targeter nucleic acid and a modulator nucleic acid, and (b) a DNA comprising a regulatory element operably linked to a Cas coding sequence. The DNA can be provided in a plasmid, viral vector, or any other form described in the “CRISPR Expression Systems” subsection. Such delivery method may result in constitutive expression of Cas protein in the target cell (e.g., if the DNA is maintained in the cell in an episomal vector or is integrated into the genome), and may increase the risk of off-targeting which is undesirable when the Cas protein has nuclease activity. Notwithstanding, this approach is useful when the Cas protein comprises a non-nuclease effector (e.g., a transcriptional activator or repressor). It is also useful for research purposes and for genome editing of plants.

### B. CRISPR Expression Systems

Also provided herein is a nucleic acid comprising a regulatory element operably linked to a nucleotide sequence encoding a guide nucleic acid disclosed herein. In certain embodiments, the nucleic acid comprises a regulatory element operably linked to a nucleotide sequence encoding a single guide nucleic acid; this nucleic acid alone can constitute a CRISPR expression system. In certain embodiments, the nucleic acid comprises a regulatory element operably linked to a nucleotide sequence encoding a targeter nucleic acid. In certain embodiments, the nucleic acid further comprises a nucleotide sequence encoding a modulator nucleic acid, wherein the nucleotide sequence encoding the modulator nucleic acid is operably linked to the same regulatory element as the nucleotide sequence encoding the targeter nucleic acid or a different regulatory element; this nucleic acid alone can constitute a CRISPR expression system.

In addition, the present invention provides a CRISPR expression system comprising: (a) a nucleic acid comprising a first regulatory element operably linked to a nucleotide sequence encoding a targeter nucleic acid and (b) a nucleic acid comprising a second regulatory element operably linked to a nucleotide sequence encoding a modulator nucleic acid.

In certain embodiments, a CRISPR expression system further comprises a nucleic acid comprising a third regulatory element operably linked to a nucleotide sequence encoding a Cas protein, such as a Cas protein disclosed herein. In certain embodiments, the Cas protein is a type V-A, type V-C, or type V-D Cas protein (e.g., Cas nuclease). In certain embodiments, the Cas protein is a type V-A Cas protein (e.g., Cas nuclease).

As used in this context, the term “operably linked” can mean that the nucleotide sequence of interest is linked to the regulatory element in a manner that allows for expression of the nucleotide sequence (e.g., in an in vitro transcription/translation system or in a host cell when the vector is introduced into the host cell).

The nucleic acids of a CRISPR expression system described above may be independently selected from various nucleic acids such as DNA (e.g., modified DNA) and RNA (e.g., modified RNA). In certain embodiments, the nucleic acids comprising a regulatory element operably linked to one or more nucleotide sequences encoding the guide nucleic acids are in the form of DNA. In certain embodiments, the nucleic acid comprising a third regulatory element operably linked to a nucleotide sequence encoding the Cas protein is in the form of DNA. The third regulatory element can be a constitutive or inducible promoter that drives the expression of the Cas protein. In other embodiments, the nucleic acid comprising a third regulatory element operably linked to a nucleotide sequence encoding the Cas protein is in the form of RNA (e.g., mRNA).

Nucleic acids of a CRISPR expression system can be provided in one or more vectors. The term “vector,” as used herein, can refer to a nucleic acid molecule capable of transporting another nucleic acid to which it has been linked. Conventional viral and non-viral based gene transfer methods can be used to introduce nucleic acids in cells, such as prokaryotic cells, eukaryotic cells, mammalian cells, or target tissues. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. Gene therapy procedures are known in the art and disclosed in Van Brunt (1988) BIOTECHNOLOGY, 6:1149; Anderson (1992) SCIENCE, 256:808; Nabel & Feigner (1993) TIBTECH, 11:211; Mitani & Caskey (1993) TIBTECH, 11:162; Dillon (1993) TIBTECH, 11:167; Miller (1992) NATURE, 357:455; Vigne, (1995) RESTORATIVE NEUROLOGY AND NEUROSCIENCE, 8:35; Kremer & Perricaudet (1995) BRITISH MEDICAL BULLETIN, 51:31; Haddada et al. (1995) CURRENT TOPICS IN MICROBIOLOGY AND IMMUNOLOGY, 199:297; Yu et al. (1994) GENE THERAPY, 1:13; and Doerfler and Bohm (Eds.) (2012) The Molecular Repertoire of Adenoviruses II: Molecular Biology of Virus-Cell Interactions. In certain embodiments, at least one of the vectors is a DNA plasmid. In certain embodiments, at least one of the vectors is a viral vector (e.g., retrovirus, adenovirus, or adeno-associated virus).

Certain vectors are capable of autonomous replication in a host cell into which they are introduced (e.g., bacterial vectors having a bacterial origin of replication and episomal mammalian vectors). Other vectors (e.g., non-episomal mammalian vectors and replication defective viral vectors) do not autonomously replicate in the host cell. Certain vectors, however, may be integrated into the genome of the host cell and thereby are replicated along with the host genome. A skilled person in the art will appreciate that different vectors may be suitable for different delivery methods and have different host tropism, and will be able to select one or more vectors suitable for the use.

The term “regulatory element,” as used herein, can refer to a transcriptional and/or translational control sequence, such as a promoter, enhancer, transcription termination signal (e.g., polyadenylation signal), internal ribosomal entry sites (IRES), protein degradation signal, or the like, that provide for and/or regulate transcription of a non-coding sequence (e.g., a targeter nucleic acid or a modulator nucleic acid) or a coding sequence (e.g., a Cas protein) and/or regulate translation of an encoded polypeptide. Such regulatory elements are described, for example, in Goeddel, GENE EXPRESSION TECHNOLOGY: METHODS IN ENZYMOLOGY, 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In certain embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and Hl promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (see, Takebe et al. (1988) MOL. CELL. BIOL., 8:466); SV40 enhancer; and the intron sequence between exons 2 and 3 of rabbit β-globin (see, O'Hare et al. (1981) PROC. NATL. ACAD. SCI. USA., 78:1527). It will be appreciated by those skilled in the art that the design of the expression vector can depend on factors such as the choice of the host cell to be transformed, the level of expression desired, etc. A vector can be introduced into host cells to produce transcripts, proteins, or peptides, including fusion proteins or peptides, encoded by nucleic acids as described herein (e.g., CRISPR transcripts, proteins, enzymes, mutant forms thereof, or fusion proteins thereof).

In certain embodiments, the nucleotide sequence encoding the Cas protein is codon optimized for expression in a prokaryotic cell, e.g., E coli, eukaryotic host cell, e.g., a yeast cell (e.g., S. cerevisiae), a mammalian cell (e.g., a mouse cell, a rat cell, or a human cell), or a plant cell. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.or.jp/codon/and these tables can be adapted in a number of ways (see, Nakamura et al. (2000) NUCL. ACIDS RES., 28:292). Computer algorithms for codon optimizing a particular sequence for expression in a particular host cell, such as Gene Forge (Aptagen; Jacobus, Pa.), arc also available. In certain embodiments, the codon optimization facilitates or improves expression of the Cas protein in the host cell.

### C. Donor Templates

Cleavage of a target nucleotide sequence in the genome of a cell by a CRISPR-Cas system or complex can activate DNA damage pathways, which may rejoin the cleaved DNA fragments by NHEJ or HDR. HDR requires a repair template, either endogenous or exogenous, to transfer the sequence information from the repair template to the target.

In certain embodiments, an engineered, non-naturally occurring system or CRISPR expression system further comprises a donor template. As used herein, the term “donor template” can refer to a nucleic acid designed to serve as a repair template at or near the target nucleotide sequence upon introduction into a cell or organism. In certain embodiments, the donor template is complementary to a polynucleotide comprising the target nucleotide sequence or a portion thereof. When optimally aligned, a donor template may overlap with one or more nucleotides of a target nucleotide sequences (e.g., about or more than about 1, 5, 10, 15, 20, 25, 30, 35, 40, or more nucleotides). The nucleotide sequence of the donor template is typically not identical to the genomic sequence that it replaces. Rather, the donor template may contain one or more substitutions, insertions, deletions, inversions, or rearrangements with respect to the genomic sequence, so long as sufficient homology is present to support homology-directed repair. In certain embodiments, the donor template comprises a non-homologous sequence flanked by two regions of homology (i.e., homology arms), such that homology-directed repair between the target DNA region and the two flanking sequences results in insertion of the non-homologous sequence at the target region. In certain embodiments, the donor template comprises a non-homologous sequence 10-100 nucleotides, 50-500 nucleotides, 100-1,000 nucleotides, 200-2,000 nucleotides, or 500-5,000 nucleotides in length positioned between two homology arms.

Generally, the homologous region(s) of a donor template has at least 50% sequence identity to a genomic sequence with which recombination is desired. The homology arms are designed or selected such that they are capable of recombining with the nucleotide sequences flanking the target nucleotide sequence under intracellular conditions. In certain embodiments, where HDR of the non-target strand is desired, the donor template comprises a first homology arm homologous to a sequence 5′ to the target nucleotide sequence and a second homology arm homologous to a sequence 3′ to the target nucleotide sequence. In certain embodiments, the first homology arm is at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to a sequence 5′ to the target nucleotide sequence. In certain embodiments, the second homology arm is at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to a sequence 3′ to the target nucleotide sequence. In certain embodiments, when the donor template sequence and a polynucleotide comprising a target nucleotide sequence are optimally aligned, the nearest nucleotide of the donor template is within about 1, 5, 10, 15, 20, 25, 50, 75, 100, 200, 300, 400, 500, 1000, 2000, 3000, 4000, or more nucleotides from the target nucleotide sequence.

In certain embodiments, the donor template further comprises an engineered sequence not homologous to the sequence to be repaired. Such engineered sequence can harbor a barcode and/or a sequence capable of hybridizing with a donor template-recruiting sequence disclosed herein.

In certain embodiments, the donor template further comprises one or more mutations relative to the genomic sequence, wherein the one or more mutations reduce or prevent cleavage, by the same CRISPR-Cas system, of the donor template or of a modified genomic sequence with at least a portion of the donor template sequence incorporated. In certain embodiments, in the donor template, the PAM adjacent to the target nucleotide sequence and recognized by the Cas nuclease is mutated to a sequence not recognized by the same Cas nuclease. In certain embodiments, in the donor template, the target nucleotide sequence (e.g., the seed region) is mutated. In certain embodiments, the one or more mutations are silent with respect to the reading frame of a protein-coding sequence encompassing the mutated sites.

The donor template can be provided to the cell as single-stranded DNA, single-stranded RNA, double-stranded DNA, or double-stranded RNA. It is understood that a CRISPR-Cas system, such as a system disclosed herein, may possess nuclease activity to cleave the target strand, the non-target strand, or both. When HDR of the target strand is desired, a donor template having a nucleic acid sequence complementary to the target strand is also contemplated.

The donor template can be introduced into a cell in linear or circular form. If introduced in linear form, the ends of the donor template may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends (see, for example, Chang et al. (1987) PROC. NATL. ACAD SCI USA, 84:4959; Nehls et al. (1996) SCIENCE, 272:886; see also the chemical modifications for increasing stability and/or specificity of RNA disclosed supra). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor template, additional lengths of sequence may be included outside of the regions of homology that can be degraded without impacting recombination.

A donor template can be a component of a vector as described herein, contained in a separate vector, or provided as a separate polynucleotide, such as an oligonucleotide, linear polynucleotide, or synthetic polynucleotide. In certain embodiments, the donor template is a DNA. In certain embodiments, a donor template is in the same nucleic acid as a sequence encoding the single guide nucleic acid, a sequence encoding the targeter nucleic acid, a sequence encoding the modulator nucleic acid, and/or a sequence encoding the Cas protein, where applicable. In certain embodiments, a donor template is provided in a separate nucleic acid. A donor template polynucleotide may be of any suitable length, such as about or at least about 50, 75, 100, 150, 200, 500, 1000, 2000, 3000, 4000, or more nucleotides in length.

A donor template can be introduced into a cell as an isolated nucleic acid. Alternatively, a donor template can be introduced into a cell as part of a vector (e.g., a plasmid) having additional sequences such as, for example, replication origins, promoters and genes encoding antibiotic resistance, that are not intended for insertion into the DNA region of interest. Alternatively, a donor template can be delivered by viruses (e.g., adenovirus, adeno-associated virus (AAV)). In certain embodiments, the donor template is introduced as an AAV, e.g., a pseudotyped AAV. The capsid proteins of the AAV can be selected by a person skilled in the art based upon the tropism of the AAV and the target cell type. For example, in certain embodiments, the donor template is introduced into a hepatocyte as AAV8 or AAV9. In certain embodiments, the donor template is introduced into a hematopoietic stem cell, a hematopoietic progenitor cell, or a T lymphocyte (e.g., CD8+T lymphocyte) as AAV6 or an AAVHSC (see, U.S. Pat. No. 9,890,396). It is understood that the sequence of a capsid protein (VP1, VP2, or VP3) may be modified from a wild-type AAV capsid protein, for example, having at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, or at least 99%) sequence identity to a wild-type AAV capsid sequence.

The donor template can be delivered to a cell (e.g., a primary cell) by various delivery methods, such as a viral or non-viral method disclosed herein. In certain embodiments, a non-viral donor template is introduced into the target cell as a naked nucleic acid or in complex with a liposome or poloxamer. In certain embodiments, a non-viral donor template is introduced into the target cell by electroporation. In other embodiments, a viral donor template is introduced into the target cell by infection. The engineered, non-naturally occurring system can be delivered before, after, or simultaneously with the donor template (see, International (PCT) Application Publication No. WO 2017/053729). A skilled person in the art will be able to choose proper timing based upon the form of delivery (consider, for example, the time needed for transcription and translation of RNA and protein components) and the half-life of the molecule(s) in the cell. In particular embodiments, where the CRISPR-Cas system including the Cas protein is delivered by electroporation (e.g., as an RNP), the donor template (e.g., as an AAV) is introduced into the cell within 4 hours (e.g., within 1, 2, 3, 4, 5, 6, 7, 8, 9, 10, 11, 12, 13, 14, 15, 16, 17, 18, 19, 20, 25, 30, 35, 40, 45, 50, 55, 60, 90, 120, 150, 180, 210, or 240 minutes) after the introduction of the engineered, non-naturally occurring system.

In certain embodiments, the donor template is conjugated covalently to a modulator nucleic acid. Covalent linkages suitable for this conjugation are known in the art and are described, for example, in U.S. Pat. No. 9,982,278 and Savic et al. (2018) ELIFE 7: e33761. In certain embodiments, the donor template is covalently linked to a modulator nucleic acid (e.g., the 5′ end of the modulator nucleic acid) through an internucleotide bond. In certain embodiments, the donor template is covalently linked to a modulator nucleic acid (e.g., the 5′ end of the modulator nucleic acid) through a linker.

In certain embodiments, the donor template can comprise any nucleic acid chemistry. In certain embodiments, the donor template can comprise DNA and/or RNA nucleotides. In certain embodiments, the donor template can comprise single-stranded DNA, linear single-stranded RNA, linear double-stranded DNA, linear double-stranded RNA, circular single-stranded DNA, circular single-stranded RNA, circular double-stranded DNA, or circular double-stranded RNA. In certain embodiments, the donor template comprises a mutation in a PAM sequence to partially or completely abolish binding of the RNP to the DNA. In certain embodiments, the donor template is present at a concentration of at least 0.05, 0.01, 0.2, 0.3, 0.4, 0.5, 0.6, 0.7, 0.8, 0.9, 1, 1.25, 1.5, 1.75, 2, 3, or 4, and/or no more than 0.01, 0.2, 0.3, 0.4, 0.5, 0.6, 0.7, 0.8, 0.9, 1, 1.25, 1.5, 1.75, 2, 3, 4, or 5 μg μL-1, for example 0.01-5 μg μL-1. In certain embodiments, the donor template comprises one or more promoters. In certain embodiments, the donor template comprises a promoter that shares at least 70%, at least 75%, at least 80%, at least 85%, at least 90%, at least 95%, at least 99.5% sequence identity with any one of SEQ ID NOS: 78-85 of Table 6.

TABLE 6
Promoter sequences

| | SEQ | |
|---|---|---|
| | ID | |
| Name | NO | Sequence |
| CMV | 78 | CGTTACATAACTTACGGTAAATGGCCCGCCTGGCTGACCGCCCAACGACCCCC |
| | | GCCCATTGACGTCAATAATGACGTATGTTCCCATAGTAACGCCAATAGGGACT |
| | | TTCCATTGACGTCAATGGGTGGAGTATTTACGGTAAACTGCCCACTTGGCAGT |
| | | ACATCAAGTGTATCATATGCCAAGTACGCCCCCTATTGACGTCAATGACGGTA |
| | | AATGGCCCGCCTGGCATTATGCCCAGTACATGACCTTATGGGACTTTCCTACT |
| | | TGGCAGTACATCTACGTATTAGTCATCGCTATTACCATGGTGATGCGGTTTTG |
| | | GCAGTACATCAATGGGCGTGGATAGCGGTTTGACTCACGGGGATTTCCAAGTC |
| | | TCCACCCCATTGACGTCAATGGGAGTTTGTTTTGGCACCAAAATCAACGGGAC |
| | | TTTCCAAAATGTCGTAACAACTCCGCCCCATTGACGCAAATGGGCGGTAGGCG |
| | | TGTACGGTGGGAGGTCTATATAAGCAGAGCT |
| SCP | 79 | GTACTTATATAAGGGGGTGGGGGCGCGTTCGTCCTCAGTCGCGATCGAACACT |
| | | CGAGCCGAGCAGACGTGCCTACGGACCG |
| CMVe- | 80 | CGTTACATAACTTACGGTAAATGGCCCGCCTGGCTGACCGCCCAACGACCCCC |
| SCP | | GCCCATTGACGTCAATAATGACGTATGTTCCCATAGTAACGCCAATAGGGACT |
| | | TTCCATTGACGTCAATGGGTGGAGTATTTACGGTAAACTGCCCACTTGGCAGT |
| | | ACATCAAGTGTATCATATGCCAAGTACGCCCCCTATTGACGTCAATGACGGTA |
| | | AATGGCCCGCCTGGCATTATGCCCAGTACATGACCTTATGGGACTTTCCTACT |
| | | TGGCAGTACATCTACGTATTAGTCATCGCTATTACCATGGTACTTATATAAGG |
| | | GGGGGGGGCGCGTTCGTCCTCAGTCGCGATCGAACACTCGAGCCGAGCAGAC |
| | | GTGCCTACGGACCG |
| CMV | 81 | TCAATATTGGCCATTAGCCATATTATTCATTGGTTATATAGCATAAATCAATA |
| max | | TTGGCTATTGGCCATTGCATACGTTGTATCTATATCATAATATGTACATTTAT |
| | | ATTGGCTCATGTCCAATATGACCGCCATGTTGGCATTGATTATTGACTAGTTA |
| | | TTAATAGTAATCAATTACGGGGTCATTAGTTCATAGCCCATATATGGAGTTCC |
| | | GCGTTACATAACTTACGGTAAATGGCCCGCCTGGCTGACCGCCCAACGACCCC |
| | | CGCCCATTGACGTCAATAATGACGTATGTTCCCATAGTAACGCCAATAGGGAC |
| | | TTTCCATTGACGTCAATGGGTGGAGTATTTACGGTAAACTGCCCACTTGGCAG |
| | | TACATCAAGTGTATCATATGCCAAGTCCGCCCCCTATTGACGTCAATGACGGT |
| | | AAATGGCCCGCCTGGCATTATGCCCAGTACATGACCTTACGGGACTTTCCTAC |
| | | TTGGCAGTACATCTACGTATTAGTCATCGCTATTACCATGGTGATGCGGTTTT |
| | | GGCAGTACACCAATGGGCGTGGATAGCGGTTTGACTCACGGGGATTTCCAAGT |
| | | CTCCACCCCATTGACGTCAATGGGAGTTTGTTTTGGCACCAAAATCAACGGGA |
| | | CTTTCCAAAATGTCGTAATAACCCCGCCCCGTTGACGCAAATGGGCGGTAGGC |
| | | GTGTACGGTGGGAGGTCTATATAAGCAGAGGTCGTTTAGTGAACCGTCAGATC |
| | | ACTAGTAGCTTTATTGCGGTAGTTTATCACAGTTAAATTGCTAACGCAGTCAG |
| | | TGCTCGACTGATCACAGGTAAGTATCAAGGTTACAAGACAGGTTTAAGGAGGC |
| | | CAATAGAAACTGGGCTTGTCGAGACAGAGAAGATTCTTGCGTTTCTGATAGGC |
| | | ACCTATTGGTCTTACTGACATCCACTTTGCCTTTCTCTCCACAGGG |
| JET | 82 | GAATTCGGGCGGAGTTAGGGCGGAGCCAATCAGCGTGCGCCGTTCCGAAAGTT |
| | | GCCTTTTATGGCTGGGCGGAGAATGGGCGGTGAACGCCGATGATTATATAAGG |
| | | ACGCGCCGGGTGTGGCACAGCTAGTTCCGTCGCAGCCGGGATTTGGGTCGCGG |
| | | TTCTTGTTTGTGGATCCCTGTGATCGTCACTTGACA |
| CAG | 83 | ATCTCGACTAGTTATTAATAGTAATCAATTACGGGGTCATTAGTTCATAGCCC |
| | | ATATATGGAGTTCCGCGTTACATAACTTACGGTAAATGGCCCGCCTGGCTGAC |
| | | CGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAAC |
| | | TGCCCACTTGGCAGTACATCAAGTGTATCATATGCCAAGTACGCCCCCTATTG |
| | | ACGTCAATGACGGTAAATGGCCCGCCTGGCATTATGCCCAGTACATGACCTTA |
| | | TGGGACTTTCCTACTTGGCAGTACATCTACGTATTAGTCATCGCTATTACCAT |
| | | GGTCGAGGTGAGCCCCACGTTCTGCTTCACTCTCCCCATCTCCCCCCCCTCCC |
| | | CACCCCCAATTTTGTATTTATTTATTTTTTAATTATTTTGTGCAGCGATGGGG |
| | | GCGGGGGGGGGGGGGGGGCGCGCGCCAGGCGGGGCGGGGCGGGGCGAGGGGCG |
| | | GGGCGGGGCGAGGCGGAGAGGTGCGGCGGCAGCCAATCAGAGCGGCGCGCTCC |
| | | GAAAGTTTCCTTTTATGGCGAGGCGGCGGCGGCGGCGGCCCTATAAAAAGCGA |
| | | AGCGCGCGGCGGGCGGGGAGTCGCTGCGACGCTGCCTTCGCCCCGTGCCCCGC |
| | | TCCGCCGCCGCCTCGCGCCGCCCGCCCCGGCTCTGACTGACCGCGTTACTCCC |
| | | ACAGGTGAGCGGGCGGGACGGCCCTTCTCCTCCGGGCTGTAATTAGCGCTTGG |
| | | TTTAATGACGGCTTGTTTCTTTTCTGTGGCTGCGTGAAAGCCTTGAGGGGCTC |
| | | CGGGAGGGCCCTTTGTGCGGGGGGAGCGGCTCGGGGGGTGCGTGCGTGTGTGT |
| | | GTGCGTGGGGAGCGCCGCGTGCGGCTCCGCGCTGCCCGGCGGCTGTGAGCGCT |
| | | GCGGGCGCGGCGCGGGGCTTTGTGCGCTCCGCAGTGTGCGCGAGGGGAGCGCG |
| | | GCCGGGGGCGGTGCCCCGCGGTGCGGGGGGGGCTGCGAGGGGAACAAAGGCTG |
| | | CGTGCGGGGTGTGTGCGTGGGGGGGTGAGCAGGGGGTGTGGGCGCGTCGGTCG |
| | | GGCTGCAACCCCCCCTGCACCCCCCTCCCCGAGTTGCTGAGCACGGCCCGGCT |
| | | TCGGGTGCGGGGCTCCGTACGGGGCGTGGCGCGGGGCTCGCCGTGCCGGGCGG |
| | | GGGGTGGCGGCAGGTGGGGGTGCCGGGCGGGGCGGGGCCGCCTCGGGCCGGGG |
| | | AGGGCTCGGGGGAGGGGCGCGGCGGCCCCCGGAGCGCCGGCGGCTGTCGAGGC |
| | | GCGGCGAGCCGCAGCCATTGCCTTTTATGGTAATCGTGCGAGAGGGCGCAGGG |
| | | ACTTCCTTTGTCCCAAATCTGTGCGGAGCCGAAATCTGGGAGGCGCCGCCGCA |
| | | CCCCCTCTAGCGGGCGCGGGGCGAAGCGGTGCGGCGCCGGCAGGAAGGAAATG |
| | | GGCGGGGAGGGCCTTCGTGCGTCGCCGCGCCGCCGTCCCCTTCTCCCTCTCCA |
| | | GCCTCGGGGCTGTCCGCGGGGGGACGGCTGCCTTCGGGGGGGACGGGGCAGGG |
| | | CGGGGTTCGGCTTCTGGCGTGTGACCGGCGGCTCTAGAGCCTCTGCTAACCAT |
| | | GTTCATGCCTTCTTCTTTTTCCTACAGCTCCTGGGCAACGTGCTGGTTATTGT |
| | | GCTGTCTCATCATTTTGGCAAAGAATT |
| PGK | 84 | GGGGTTGGGGTTGCGCCTTTTCCAAGGCAGCCCTGGGTTTGCGCAGGGACGCG |
| | | GCTGCTCTGGGCGTGGTTCCGGGAAACGCAGCGGCGCCGACCCTGGGTCTCGC |
| | | ACATTCTTCACGTCCGTTCGCAGCGTCACCCGGATCTTCGCCGCTACCCTTGT |
| | | GGGCCCCCCGGCGACGCTTCCTGCTCCGCCCCTAAGTCGGGAAGGTTCCTTGC |
| | | GGTTCGCGGCGTGCCGGACGTGACAAACGGAAGCCGCACGTCTCACTAGTACC |
| | | CTCGCAGACGGACAGCGCCAGGGAGCAATGGCAGCGCGCCGACCGCGATGGGC |
| | | TGTGGCCAATAGCGGCTGCTCAGCAGGGCGCGCCGAGAGCAGCGGCCGGGAAG |
| | | GGGCGGTGCGGGAGGCGGGGTGTGGGGCGGTAGTGTGGGCCCTGTTCCTGCCC |
| | | GCGCGGTGTTCCGCATTCTGCAAGCCTCCGGAGCGCACGTCGGCAGTCGGCTC |
| | | CCTCGTTGACCGAATCACCGACCTCTCTCCCCAG |
| EF- | 85 | GAATTCAGGCTCCGGTGCCCGTCAGTGGGCAGAGCGCACATCGCCCACAGTCC |
| 1a | | CCGAGAAGTTGGGGGGAGGGGTCGGCAATTGAACCGGTGCCTAGAGAAGGTGG |
| | | CGCGGGGTAAACTGGGAAAGTGATGTCGTGTACTGGCTCCGCCTTTTTCCCGA |
| | | GGGTGGGGGAGAACCGTATATAAGTGCAGTAGTCGCCGTGAACGTTCTTTTTC |
| | | GCAACGGGTTTGCCGCCAGAACACAGGTAAGTGCCGTGTGTGGTTCCCGCGGG |
| | | CCTGGCCTCTTTACGGGTTATGGCCCTTGCGTGCCTTGAATTACTTCCACCTG |
| | | GCTGCAGTACGTGATTCTTGATCCCGAGCTTCGGGTTGGAAGTGGGTGGGAGA |
| | | GTTCGAGGCCTTGCGCTTAAGGAGCCCCTTCGCCTCGTGCTTGAGTTGAGGCC |
| | | TGGCCTGGGCGCTGGGGCCGCCGCGTGCGAATCTGGTGGCACCTTCGCGCCTG |
| | | TCTCGCTGCTTTCGATAAGTCTCTAGCCATTTAAAATTTTTGATGACCTGCTG |
| | | CGACGCTTTTTTTCTGGCAAGATAGTCTTGTAAATGCGGGCCAAGATCTGCAC |
| | | ACTGGTATTTCGGTTTTTGGGGCCGCGGGCGGCGACGGGGCCCGTGCGTCCCA |
| | | GCGCACATGTTCGGCGAGGCGGGGCCTGCGAGCGCGGCCACCGAGAATCGGAC |
| | | GGGGGTAGTCTCAAGCTGGCCGGCCTGCTCTGGTGCCTGGTCTCGCGCCGCCG |
| | | TGTATCGCCCCGCCCTGGGCGGCAAGGCTGGCCCGGTCGGCACCAGTTGCGTG |
| | | AGCGGAAAGATGGCCGCTTCCCGGCCCTGCTGCAGGGAGCTCAAAATGGAGGA |
| | | CGCGGCGCTCGGGAGAGCGGGCGGGTGAGTCACCCACACAAAGGAAAAGGGCC |
| | | TTTCCGTCCTCAGCCGTCGCTTCATGTGACTCCACGGAGTACCGGGCGCCGTC |
| | | CAGGCACCTCGATTAGTTCTCGAGCTTTTGGAGTACGTCGTCTTTAGGTTGGG |
| | | GGGAGGGGTTTTATGCGATGGAGTTTCCCCACACTGAGTGGGTGGAGACTGAA |
| | | GTTAGGCCAGCTTGGCACTTGATGTAATTCTCCTTGGAATTTGCCCTTTTTGA |
| | | GTTTGGATCTTGGTTCATTCTCAAGCCTCAGACAGTGGTTCAAAGTTTTTTTC |
| | | TTCCATTTCAGGTGTCGTGACATCATTTT |

### D. Efficiency and Specificity

An engineered, non-naturally occurring system can be evaluated in terms of efficiency and/or specificity in nucleic acid targeting, cleavage, or modification.

In certain embodiments, an engineered, non-naturally occurring system has high efficiency. For example, in certain embodiments, at least 1, 1.5, 2, 2.5, 3, 4, 5, 10, 15, 20, 25, 30, 35, 40, 45, 50, 55, 60, 65, 70, 75, 80, 85, 90, 95, 96, 97, 98, 99, or 100% of a population of nucleic acids having the target nucleotide sequence and a cognate PAM, when contacted with the engineered, non-naturally occurring system, is targeted, cleaved, or modified. In certain embodiments, the genomes of at least 5, 10, 15, 20, 25, 30, 35, 40, 45, 50, 55, 60, 65, 70, 75, 80, 85, 90, 95, 96, 97, 98, 99, or 100% of a population of cells, when the engineered, non-naturally occurring system is delivered into the cells, are targeted, cleaved, or modified.

It has been observed that for a given spacer sequence, the occurrence of on-target events and the occurrence of off-target events are generally correlated. For certain therapeutic purposes, lower on-target efficiency can be tolerated and low off-target frequency is more desirable. For example, when editing or modifying a proliferating cell that will be delivered to a subject and proliferate in vivo, tolerance to off-target events is low. Prior to delivery, it is possible to assess the on-target and off-target events, thereby selecting one or more colonies that have the desired edit or modification and lack any undesired edit or modification. Notwithstanding, the on-target efficiency may need to meet a certain standard to be suitable for therapeutic use. High editing efficiency in a standard CRISPR-Cas system allows tuning of the system, for example, by reducing the binding of the guide nucleic acids to the Cas protein, without losing therapeutic applicability.

In certain embodiments, when a population of nucleic acids having the target nucleotide sequence and a cognate PAM is contacted with the engineered, non-naturally occurring system disclosed herein, the frequency of off-target events (e.g., targeting, cleavage, or modification, depending on the function of the CRISPR-Cas system) is reduced. Methods of assessing off-target events were summarized in Lazzarotto et al. (2018) NAT PROTOC. 13 (11): 2615-42, and include discovery of in situ Cas off-targets and verification by sequencing (DISCOVER-seq) as disclosed in Wienert et al. (2019) SCIENCE 364 (6437): 286-89; genome-wide unbiased identification of double-stranded breaks (DSBs) enabled by sequencing (GUIDE-seq) as disclosed in Kleinstiver et al. (2016) NAT. BIOTECH. 34:869-74; circularization for in vitro reporting of cleavage effects by sequencing (CIRCLE-seq) as described in Kocak et al. (2019) NAT. BIOTECH. 37:657-66. In certain embodiments, the off-target events include targeting, cleavage, or modification at a given off-target locus (e.g., the locus with the highest occurrence of off-target events detected). In certain embodiments, the off-target events include targeting, cleavage, or modification at all the loci with detectable off-target events, collectively.

In certain embodiments, genomic mutations are detected in no more than 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, or 5% of the cells at any off-target loci (in aggregate). In certain embodiments, the ratio of the percentage of cells having an on-target event to the percentage of cells having any off-target event (e.g., the ratio of the percentage of cells having an on-target editing event to the percentage of cells having a mutation at any off-target loci) is at least 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000. It is understood that genetic variation may be present in a population of cells, for example, by spontaneous mutations, and such mutations are not included as off-target events.

### E. Multiplexing

The method of targeting, editing, and/or modifying a genomic DNA disclosed herein can be conducted in multiplicity. For example, a library of targeter nucleic acids can be used to target multiple genomic loci; a library of donor templates can also be used to generate multiple insertions, deletions, and/or substitutions. The multiplex assay can be conducted in a screening method wherein each separate cell culture (e.g., in a well of a 96-well plate or a 384-well plate) is exposed to a different guide nucleic acid having a different targeter stem sequence and/or a different donor template. The multiplex assay can also be conducted in a selection method wherein a cell culture is exposed to a mixed population of different guide nucleic acids and/or donor templates, and the cells with desired characteristics (e.g., functionality) are enriched or selected by advantageous survival or growth, resistance to a certain agent, expression of a detectable protein (e.g., a fluorescent protein that is detectable by flow cytometry), etc.

In certain embodiments, the plurality of guide nucleic acids and/or the plurality of donor templates are designed for saturation editing. For example, in certain embodiments, each nucleotide position in a sequence of interest is systematically modified with each of all four traditional bases, A, T, G and C. In other embodiments, at least one sequence in each gene from a pool of genes of interest is modified, for example, according to a CRISPR design algorithm. In certain embodiments, each sequence from a pool of exogenous elements of interest (e.g., protein coding sequences, non-protein coding genes, regulatory elements) is inserted into one or more given loci of the genome.

It is understood that the multiplex methods suitable for the purpose of carrying out a screening or selection method, which is typically conducted for research purposes, may be different from the methods suitable for therapeutic purposes. For example, constitutive expression of certain elements (e.g., a Cas nuclease and/or a guide nucleic acid) may be undesirable for therapeutic purposes due to the potential of increased off-targeting. Conversely, for research purposes, constitutive expression of a Cas nuclease and/or a guide nucleic acid may be desirable. For example, the constitutive expression provides a large window during which other elements can be introduced. When a stable cell line is established for the constitutive expression, the number of exogenous elements that need to be co-delivered into a single cell is also reduced. Therefore, constitutive expression of certain elements can increase the efficiency and reduce the complexity of a screening or selection process. Inducible expression of certain elements of the system disclosed herein may also be used for research purposes given similar advantages. Expression may be induced by an exogenous agent (e.g., a small molecule) or by an endogenous molecule or complex present in a particular cell type (e.g., at a particular stage of differentiation). Methods known in the art, such as those described herein, can be used for constitutively or inducibly expressing one or more elements. For example, the specificity of CRISPR nucleases is at least partially dictated by the uniqueness of the spacer (in combination with spacer sequence's proximity to a requisite PAM) and its off-target score can be calculated with algorithms, such as crispr.mit.edu (Hsu et al. (2013) NAT. BIOTECH. 31:827-832). The highest possible score is 100, which shows probability for high specificity and few off targets. Because our SHS library targets intergenic regions, the algorithm for gRNA prediction should be able to make alignments with repeated regions and low-complexity sequences.

It is further understood that despite the need to introduce multiple elements—the single guide nucleic acid and the Cas protein; or the targeter nucleic acid, the modulator nucleic acid, and the Cas protein—these elements can be delivered into the cell as a single complex of pre-formed RNP. Therefore, the efficiency of the screening or selection process can also be achieved by pre-assembling a plurality of RNP complexes in a multiplex manner.

In certain embodiments, the method disclosed herein further comprises a step of identifying a guide nucleic acid, a Cas protein, a donor template, or a combination of two or more of these elements from the screening or selection process. A set of barcodes may be used, for example, in the donor template between two homology arms, to facilitate the identification. In specific embodiments, the method further comprises harvesting the population of cells; selectively amplifying a genomic DNA or RNA sample including the target nucleotide sequence(s) and/or the barcodes; and/or sequencing the genomic DNA or RNA sample and/or the barcodes that has been selectively amplified.

In addition, the present invention provides a library comprising a plurality of guide nucleic acids, such as a plurality of guide nucleic acids disclosed herein. In another aspect, the present invention provides a library comprising a plurality of nucleic acids each comprising a regulatory element operably linked to a different guide nucleic acid such as a different guide nucleic acid disclosed herein. These libraries can be used in combination with one or more Cas proteins or Cas-coding nucleic acids, such as disclosed herein, and/or one or more donor templates, such as disclosed herein, for a screening or selection method.

### F. Genomic Safe Harbors

Genome engineering is an area of research seeking to modify genes of living organisms to improve our understanding of gene function and to develop methods for genome engineering that treat genetic or acquired diseases, among many others. To modify the genome of target cells, skilled artisans use one or more available tools to introduce changes into the genome at targeted locations to modify the sequence of a target polynucleotide, e.g., a target gene, in desired ways, e.g., modulate gene expression, modulate gene sequences, remove gene sequences, introduce genes, e.g., exogenous DNA, e.g., transgenes, and the like. Efficient transgene insertion may be accomplished through non-precise methods including but not limited to viral vectors, such as, retroviral vectors, e.g., adeno-associated virus (AAV) and the like, or precise methods including but not limited to guided nucleases, such as, zinc finger nucleases (ZFNs), transcription activator-like effector nucleases (TALENs), homing endonucleases, e.g., restriction endonucleases, or nucleic acid-guided nuclease, e.g., CRISPR-cas, e.g., Cas9 and Cas12a and engineered versions thereof.

Exogenous genes, e.g., transgenes, inserted into the genome of a target human cell either randomly, e.g., through retroviral vectors, or in a targeted manner, e.g., through the action of a nucleic acid-guided nuclease, such as Cas, may interact with other genomic elements in unpredictable ways. Due to the complex transcriptional regulation of genes in mammalian cells through networks of cis and trans regulatory elements, such as proximal and distal enhancers, and multiple transcription factors, attempts to alter the default genomic architecture by integration of exogenous DNA, e.g., transgenes, or synthetic sequences can affect the expression of the transgene itself leading to complete attenuation or complete silencing, and/or the expression of both nearby and distant endogenous genes that can, e.g., compromise the safety checkpoints that healthy cells have including dysregulation of expression of key genes, such as oncogenes and tumor suppressor genes, that can alter cellular behavior in dramatic ways, i.e., promoting clonal expansion or malignant transformation of the host.

Gene integration next to regulatory elements of proto-oncogenes has been shown to cause oncogenic transformation, which is particularly important when engineering cells for therapeutic applications. Therefore, the identification of suitable target polynucleotide comprising a target nucleotide sequence in the human genome wherein the insertion of a transgene leads to suitable expression of the transgene without disruption of neighboring genes is desired. In particular, for gene and cell therapy applications, suitable target polynucleotide comprising a target nucleotide sequence in the human genome wherein the insertion of a transgene leads to sufficient expression of the transgene in a therapeutic cell e.g., a T cell, e.g., a CAR T cell; or precursor cell, e.g., a stem cell, such as a hematopoietic stem cell, without malignant transformation or any other disruption that would be harmful to an individual after implantation is desired.

Expression of exogenous genes, e.g., transgenes, in desired cell types and/or developmental/differentiation stages relies on integration into suitable target polynucleotide comprising a target nucleotide sequence that results in sufficient expression, to a degree sufficient for the intended purpose, from the candidate locus. Expression from a specific genomic site can be affected by many factors including but not limited to cell type and differentiation stage, as one or more components of the target polynucleotide get activated during differentiation while others get silenced, and changes in chromatin architecture. Therefore, the identification of suitable target polynucleotides comprising a target nucleotide sequence in the human genome wherein insertion of exogenous DNA, e.g., a transgene, leads to sufficient expression in the target human cell, and, in the case of stem cells, the expression is maintained at a sufficient level through (1) differentiation and (2) through clonal expansion is desired. The current disclosure provides significant advances in the ability engineer human genomes by providing compositions and methods for targeting and delivering exogenous genes, e.g., transgenes, to the suitable target polynucleotide comprising a target nucleotide sequence.

Provided herein are compositions and methods for genome engineering. Certain embodiments comprise compositions. Certain embodiments comprise composition for editing genomes. embodiments disclosed herein concern novel guide nucleic acids (gNAs), e.g., gRNAs, that are complementary to a target nucleotide sequence in a target polynucleotide. As used herein, a “target polynucleotide,” includes a polynucleotide in which a target nucleotide sequence is located. As used herein, a “target nucleotide sequence” includes a sequence to which a guide sequence can bind, e.g., has complementarity to, where binding between a target nucleotide sequence and a guide sequence may allow the activity of a nucleic acid-guided nuclease complex. Further embodiments disclosed herein concern novel gNAs, e.g., gRNAs, that are complementary to a target nucleotide sequence in a target polynucleotide into which insertion of exogenous DNA, e.g., a transgene, doesn't negatively affect the cell, e.g., significantly affect the expression of one or more endogenous genes or result in a malignant transformation of the cell. In further embodiments disclosed herein, gene expression demonstrated in the human target cell is maintained through differentiation of the human target cell and/or through proliferation in the one or more progeny cells at a level sufficient for the ultimate use of the cells. Certain embodiments disclosed herein concern novel nucleic acid-guided nuclease complexes, e.g., RNPs, such as Cas bound to a gNA, that are complementary to a target nucleotide sequence within a target polynucleotide and hydrolyze the phosphodiester back bone (also referred as cleave or cut) in at least one position on at least one strand of the target polynucleotide. Certain embodiments disclosed herein concern methods for selecting and using gNAs, e.g., gRNAs, for genome engineering. Certain embodiments concern methods for using gNAs that are complementary to a target nucleotide sequence within a target polynucleotide, synthesizing the gNA and nucleic-acid-guided nuclease, and/or combining the nucleic guided nuclease with the gNA to form a nucleic acid-guided nuclease complex, e.g., RNP. Certain embodiments disclosed herein concern methods. Certain embodiments disclosed herein concern methods for engineering genomes. Certain embodiments disclosed herein concern methods where a nucleic acid-guided nuclease complex, e.g., RNP, is introduced, e.g., transfected, into a human target cell along with a donor template, e.g., an exogenous DNA, e.g., a transgene, in which the nucleic-acid guided nuclease cleaves the backbone at a least one position in at least one of the strands of the target polynucleotide and the donor template is used to repair the cleaved target polynucleotide, introducing at least a portion of the donor template into the target polynucleotide. As used herein, “exogenous DNA” or a “transgene” includes any gene, natural or synthetic, which is introduced into the genome of an organism or cell to which it is not endogenous. The transgene may or may not retain the ability to be expressed and/or produce RNA or protein in the human target cell. The transgene may or may not alter the resulting phenotype of the human target cell. Certain embodiments include human target cells, e.g., a eukaryotic cell, e.g., a mammalian cell, such as a human cell, for example a stem cell or an immune cell, generated through a method where the nucleic acid-guided nuclease complex, e.g., RNP, is introduced, e.g., transfected, into a human target cell along with a donor template, e.g., as an exogenous DNA or a transgene, such as a chimeric antigen receptor (CAR), in which the nucleic-acid guided nuclease cleaves at or near a targets sequence in a target polynucleotide and the donor template is used to repair the cleaved target polynucleotide introducing at least a portion of the donor template into the target polynucleotide. Certain embodiments disclosed herein include promoter sequences adjacent to an exogenous gene, e.g., a transgene; in certain cases, constructs including the promoter, when introduced into a target polynucleotide of a human target cell, e.g., an immune cell or a stem cell, maintain sufficient gene expression in the edited human target cell for the intended purpose of the cell or its progeny. In certain embodiments, the human target cell is viable after introduction of the exogenous DNA.

As used herein, a “human target cell” includes a cell into which an exogenous product, e.g., a protein, a nucleic acid, or a combination thereof, has been introduced. In certain cases, a human target cell may be used to produce a gene product from an exogenous DNA, e.g., a transgene, such as an exogenous protein, e.g., a CAR. In certain cases, a human target cell may comprise a target nucleotide sequence within target polynucleotide wherein a nucleic acid-guided nuclease hybridizes and cleaves at a site of cleavage at one or more positions on one or more strands of the target polynucleotide at or near the target nucleotide sequence.

As used herein, a “site of cleavage” includes the location or locations at which a nucleic acid-guided nuclease complex will hydrolyze the phosphodiester backbone of a single-stranded or double-stranded target polynucleotide, after binding at a target nucleotide sequence in the target polynucleotide. In certain cases in which the target polynucleotide of a nucleic acid-guided nuclease complex is double stranded, binding of the nucleic acid-guided nuclease complex to a target nucleotide sequence within the target polynucleotide can result in hydrolysis of one of the strands of the target polynucleotide at or near the target nucleotide sequence, resulting in strand cleavage. In such a case, the nucleic acid-guided nuclease complex can cleave either strand of the target polynucleotide. In certain cases, binding of the nucleic acid-guided nuclease complex to a target nucleotide sequence within a target polynucleotide can result in hydrolysis of both strands of the target polynucleotide at or near the target nucleotide sequence, resulting in cleavage of both strands. The sites of cleavage can be the same for both strands, resulting in a blunt end, or the sites of cleavage for each strand can be offset resulting in single strand overhangs, e.g., sticky ends. In certain cases, mismatches at or near the site of cleavage may or may not affect the cleavage efficiency of the nucleic acid-guided nuclease complex.

In certain cases, uncontrolled gene integration next to regulatory elements of proto-oncogenes has been shown to cause oncogenic transformation, which is particularly important.

when engineering cells for therapeutic applications. Therefore, it is desired to identify suitable target polynucleotides comprising target nucleotide sequences that result in safe, stable integration of exogenous DNA with sufficient expression in a human target cell and its resultant progeny.

Exemplary characteristics of a target nucleotide sequence that can demonstrate predictable function without potentially harmful alterations in human target cell genomic activity include one or more of (1) >150 kb, for example, >200, such as >250, and in some cases >300 kb away from a known cancer-related gene, (2) >150 kb, for example, >200, such as >250, and in some cases >300 kb away from any miRNA/other functional small RNA, (3) >10 kb, for example, >20, such as >30, and in some cases >50 kb away from any 5′ gene end, (4) >10 kb, for example, >20, such as >30, and in some cases >50 kb away from any replication origin, (5) >10 kb, for example, >20, such as >30, and in some cases >50 kb away from any ultra-conserved element, (6) demonstrating low transcriptional activity, (7) outside of a copy number variable region, (8) located in open chromatin, and (9) unique, i.e., 1 copy per genome.

In certain embodiments, provided herein are compositions. In certain embodiments, provided herein are compositions for engineering a human target cell at suitable target nucleotide sequences within a target polynucleotide of the human target cell.

In certain embodiments, a suitable target polynucleotide that comprises a target nucleotide sequence has at least one of the exemplary characteristics. In certain embodiments, a suitable target polynucleotide that comprises a target nucleotide sequence has at least two of the exemplary characteristics. In certain embodiments, a suitable target polynucleotide that comprises a target nucleotide sequence has at least three of the exemplary characteristics. In certain embodiments, a suitable target polynucleotide that comprises a target nucleotide sequence has at least four of the exemplary characteristics. In certain embodiments, a suitable target polynucleotide that comprises a target nucleotide sequence has at least five of the exemplary characteristics. In certain embodiments, a suitable target polynucleotide that comprises a target nucleotide sequence has at least six of the exemplary characteristics. In certain embodiments, a suitable target polynucleotide that comprises a target nucleotide sequence has at least seven of the exemplary characteristics. In certain embodiments, a suitable target polynucleotide that comprises a target nucleotide sequence has at least eight of the exemplary characteristics. In certain embodiments, a suitable target polynucleotide that comprises a target nucleotide sequence has all the exemplary characteristics.

In certain embodiments, a suitable target polynucleotide is >10 kb, for example, >20, such as >30, and in some cases >50 kb away from any 5′ gene end. In certain embodiments, a suitable target polynucleotide is >10 kb, for example, >20, such as >30, and in some cases >50 kb away from any 5′ gene end and further comprises at one additional exemplary characteristic. In certain embodiments, a suitable target polynucleotide is >10 kb, for example, >20, such as >30, and in some cases >50 kb away from any 5′ gene end and further comprises at least two additional exemplary characteristics. In certain embodiments, a suitable target polynucleotide is >10 kb, for example, >20, such as >30, and in some cases >50 kb away from any 5′ gene end and further comprises at least three additional exemplary characteristics. In certain embodiments, a suitable target polynucleotide is >10 kb, for example, >20, such as >30, and in some cases >50 kb away from any 5′ gene end and further comprises at least four additional exemplary characteristics. In certain embodiments, a suitable target polynucleotide is >10 kb, for example, >20, such as >30, and in some cases >50 kb away from any 5′ gene end and further comprises at least five additional exemplary characteristics. In certain embodiments, a suitable target polynucleotide is >10 kb, for example, >20, such as >30, and in some cases >50 kb away from any 5′ gene end and further comprises at least six additional exemplary characteristics. In certain embodiments, a suitable target polynucleotide is >10 kb, for example, >20, such as >30, and in some cases >50 kb away from any 5′ gene end and further comprises at least seven additional exemplary characteristics. In certain embodiments, a suitable target polynucleotide is >10 kb, for example, >20, such as >30, and in some cases >50 kb away from any 5′ gene end and further comprises all eight additional exemplary characteristics.

In certain embodiments, a suitable target polynucleotide is >150 kb, for example, >200, such as >250, and in some cases >300 kb away from a known cancer-related gene. In certain embodiments, a suitable target polynucleotide is >150 kb, for example, >200, such as >250, and in some cases >300 kb away from a known cancer-related gene and further comprises at one additional exemplary characteristic. In certain embodiments, a suitable target polynucleotide is >150 kb, for example, >200, such as >250, and in some cases >300 kb away from a known cancer-related gene and further comprises at least two additional exemplary characteristics. In certain embodiments, a suitable target polynucleotide is >150 kb, for example, >200, such as >250, and in some cases >300 kb away from a known cancer-related gene and further comprises at least three additional exemplary characteristics. In certain embodiments, a suitable target polynucleotide is >150 kb, for example, >200, such as >250, and in some cases >300 kb away from a known cancer-related gene and further comprises at least four additional exemplary characteristics. In certain embodiments, a suitable target polynucleotide is >150 kb, for example, >200, such as >250, and in some cases >300 kb away from a known cancer-related gene and further comprises at least five additional exemplary characteristics. In certain embodiments, a suitable target polynucleotide is >150 kb, for example, >200, such as >250, and in some cases >300 kb away from a known cancer-related gene and further comprises at least six additional exemplary characteristics. In certain embodiments, a suitable target polynucleotide is >150 kb, for example, >200, such as >250, and in some cases >300 kb away from a known cancer-related gene and further comprises at least seven additional exemplary characteristics. In certain embodiments, a suitable target polynucleotide is >150 kb, for example, >200, such as >250, and in some cases >300 kb away from a known cancer-related gene and further comprises all eight additional exemplary characteristics.

In certain embodiments, a suitable target polynucleotide is >150 kb, for example, >200, such as >250, and in some cases >300 kb away from a known cancer-related gene, and >10 kb, for example, >20, such as >30, and in some cases >50 kb away from any 5′ gene end. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises at least one additional exemplary characteristic. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises at least two additional exemplary characteristics. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises at least three additional exemplary characteristics. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises at least four additional exemplary characteristics. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises at least five additional exemplary characteristics. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises at least six additional exemplary characteristics. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises all seven additional exemplary characteristics.

In a preferred embodiment, a suitable target polynucleotide is >10 kb, for example, >20, such as >30, and in some cases >50 kb away from any 5′ gene end and >150, for example, >200, such as >250, and in some cases >300 kb away from a known cancer-related gene.

In certain embodiments, a suitable target polynucleotide comprising a target nucleotide sequence, e.g., for transgene insertion, may comprise any one of SEQ ID NOs: 2020-2043 of Table 7. In certain embodiments, a suitable target polynucleotide comprising a target nucleotide sequence is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or completely identical to any one of SEQ ID NOs: 2020-2043. In a preferred embodiment, a suitable target polynucleotide comprising a target nucleotide sequence is at least 98% identical to any one of SEQ ID NOs: 2020-2043. In a more preferred embodiment, a suitable target polynucleotide comprising a target nucleotide sequence is at least 99% identical to any one of SEQ ID NOs: 2020-2043.

In certain embodiments, a suitable target polynucleotide comprising a target nucleotide sequence, e.g., for transgene insertion, may comprise any one of SEQ ID NOs: 2020-2042 of Table 7. In certain embodiments, a suitable target polynucleotide comprising a target nucleotide sequence is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or completely identical to any one of SEQ ID NOs: 2020-2042. In a preferred embodiment, a suitable target polynucleotide comprising a target nucleotide sequence is at least 98% identical to any one of SEQ ID NOs: 2020-2042. In a more preferred embodiment, a suitable target polynucleotide comprising a target nucleotide sequence is at least 99% identical to any one of SEQ ID NOs: 2020-2042.

In certain embodiments, a suitable target polynucleotide comprising a target nucleotide sequence, e.g., for transgene insertion, may comprise any one of SEQ ID NOs: 2020-2041 and 2043 of Table 7. In certain embodiments, a suitable target polynucleotide comprising a target nucleotide sequence is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or completely identical to any one of SEQ ID NOs: 2020-2041 and 2043. In a preferred embodiment, a suitable target polynucleotide comprising a target nucleotide sequence is at least 98% identical to any one of SEQ ID NOs: 2020-2041 and 2043. In a more preferred embodiment, a suitable target polynucleotide comprising a target nucleotide sequence is at least 99% identical to any one of SEQ ID NOs: 2020-2041 and 2043.

In certain embodiments, a suitable target polynucleotide comprising a target nucleotide sequence, e.g., for transgene insertion, may comprise any one of SEQ ID NOs: 2020-2041 of Table 7. In certain embodiments, a suitable target polynucleotide comprising a target nucleotide sequence is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or completely identical to any one of SEQ ID NOs: 2020-2041. In a preferred embodiment, a suitable target polynucleotide comprising a target nucleotide sequence is at least 98% identical to any one of SEQ ID NOs: 2020-2041. In a more preferred embodiment, a suitable target polynucleotide comprising a target nucleotide sequence is at least 99% identical to any one of SEQ ID NOs: 2020-2041.

In certain embodiments, a suitable target polynucleotide comprising a target nucleotide sequence, e.g., for transgene insertion, may comprise at least a portion of, for example, nucleotides 1-495, 1-490, 1-485, 1-480, 1-475, 1-470, 1-465, 1-460, 1-455, 1-450, 1-445, 1-440, 1-435, 1-430, 1-425, 1-420, 1-415, 1-410, 1-405, or 1-400, of any one of SEQ ID NOs: 2020-2030 of Table 7. In certain embodiments, a suitable target polynucleotide comprising a target nucleotide sequence is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or completely identical to the portion of any one of SEQ ID NOs: 2020-2030.

In certain embodiments, a suitable target polynucleotide comprising a target nucleotide sequence, e.g., for transgene insertion, may comprise at least a portion of, for example, nucleotides 5-500, 10-500, 15-500, 20-500, 25-500, 30-500, 35-500, 40-500, 45-500, 50-500, 55-500, 60-500, 65-500, 70-500, 75-500, 80-500, 85-500, 90-500, 95-500, or 100-500, of any one of SEQ ID NOs: 2031-2041 of Table 7. In certain embodiments, a suitable target polynucleotide comprising a target nucleotide sequence is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or completely identical to the portion of any one of SEQ ID NOs: 2031-2041.

TABLE 7
suitable target polynucleotides comprising a target nucleotide sequence for
transgene insertion

| SEQ ID NO | Sequence |
|---|---|
| 2020 | GCCTCCCAAAGTGCTGAGATTATGGGCATGAGCCACCGCACCTGGCCCTGAC |
| | AAGAACCTTTGAGTTAGGTATAATGGTTCACCCCAATTTATAGATAATGAAC |
| | CCAAGTCACAGGGGAAGTGAAGTCAGTTGCCTAAGGTCAGACAGCAGTAAAT |
| | GGTTCTCTGACCCTAACTCCACTGCCTCCCTCTCATAAAAACACTGGGTGGT |
| | TACAGTGGGCCCACCTGGAGAAGTCAAGCTATTCTCTCCATCTCAAGAACAT |
| | TAATTTAATCATCCTTTTTACCATATAAGATAACATCTTCACAGGTTCTGAG |
| | GATGAGAATGTTGACATCTTTGGGTGGTCGTTATTCAGCCTATCACAGGTAT |
| | CCAGGGAAGAAAAAAGGAATTTCCAAAAAGAGAAAATACGAACATTGGGAAG |
| | GCTAATTACAGATGGTGACTACTGAAGGGTTAGTCAGAAGCATAATGGAGGC |
| | AGTGATGAGATGACAGCACAGATGCATGACTCTAGTCCCAGCAACTCCTAAA |
| | AGGTAAAGAAATGTATCCTGCCACCCTCAGCTTCTTTGGGGTGTCCTCATAA |
| | AAGAGAGGCAGTAAAGCAGAATCAGAGTCAGATAGAGAGGTTGTAAGAAGAG |
| | AAGCAGAGGTGAGTAAGCTGTGTTTCAAACCCAGAGTCAAGGCTCTTGCCCC |
| | TCTGCGGTGCTGCCGAAGCCCAGGGTGGGTGGGGACTGACATGCAACTCAGG |
| | TACTGTGTGGCAGACTTTGTGCCTTGGCATGAAACTATGCCTGCCCACAGGA |
| | AGGGGCACCATTTTCTCATTAGCTCAAAGAGACTTCTGCTGGCCAATTCCTG |
| | TCTTCTCAATACTGCAGCTCTCCAGAGACAACACTGTTCTCTATTCTCCTGT |
| | AAGTGAGGCAGAGCCTGGCAGTACCCTCTATGCCACCTCTCACTAGTACAGG |
| | TTAGCACTCAGGGTGGCCCACTGGTGTGTGTCTCAGCTGCTGGTGTGCGTGC |
| | TGGTGCAGGTAC |
| 2021 | TGGGCTGAGGGTTGTGGCTGGATCTCTTTGCATTGCCACATCCACAACAGAA |
| | TTTTGAGAAGTCCGAGAATTCTAAATTGGAGCCTGACCTTCTTCATAATAGT |
| | ATATTTGTCAAGGTAGGAGGATAAAACATTTTATTGAACAGTTTGCTAAGCT |
| | GATTTAAAATTTTCCAGCATTTAGCTATATGGTATATGGACCTCCACATGTA |
| | TGATTTCATTTATATTAAATGTCCAGAATAGACAAATCTATAATGACAATAA |
| | AGAGATTAGTAGTTGCCAGAGGCTGGGAGGAGGGGAGAAACAATGAGTGATT |
| | GCGGACGGGTGTGGGGTTTCTTTCTGGGGCGATAACAGTGTCCTGGAATTCA |
| | ATAGTGATAATGGATGCACACTGTGAATATACTAAAAGCCACTCACACTTTA |
| | AAAGTGTGGGTTTTATGGTAATTTGAATGATATATCAAGCTATCACCAAAAA |
| | TACACAATGGGAGTTCAGAAATGCCACCCCAAACTATGATGATTTGACATGC |
| | TCATTACTTTGAACTGATGTCACTTGGGGAAAAACAGATGCAGGCAGAGACT |
| | TTCTCTGAGATCTGCTTATCTGCCTAAGACAGATCAAGGGATCCTCCAAAAG |
| | GAACTCAATTGTCATGAATCCCCTCCCCTGGAACCTTATCAACCAGGGACAA |
| | TGAACTTAGATCAGAGAGGGGGAGACTGGAGGTTGACATCATGCCTAGACAG |
| | CCACCTCTTCTTCTGAGGGCTGCTCCAAGAGAACCTTTATTACTTGAGAGGC |
| | TTCTCATTTGCATAACAAGAAACCTTTGTTCACCATACACTTCCTCCCCTCA |
| | TATTCTCATAACTGGTGTCACCACCACCCACGCAGAAGTCCAAAGCCTCTAT |
| | TCCCTTCTGTACCTCAGGGTGCTATATAAGCTTCAATCATCTGACCCTTCTT |
| | TGAATCTCATATTTTGTGGGCTTGCATGGGTATGTACATAATTAAAAATGGA |
| | TTTCCTCTTGTT |
| 2022 | ATTTACACACATGCCACAGACAGAAACATTTTAATAGACCTTTGCTTATGGA |
| | AAAGTAAAGCAAAAATGTAATTCTAGAAGGGAGAAATTTTAGTCAATTAGAA |
| | AATAAGATGGTCAGGCATTGTAGCTCTCATGTGTAATCCCAGTGCTTTGGAA |
| | GGTTGAGGCGAGAGGATTGCTTGAGACCAGGAGTTTGCGACCAGCCTAGACA |
| | ACATAGCTGGTCATATAAAAAAACTTCAAAAAAATTAGCTAGCTGTAGAGCT |
| | TTCTGCCTATATTTCCAGCTACTCAAGGATGAGGCAAAAGAATCCCTTAAGC |
| | CCAGGAGGTTGAGGTTGCAGTGAACTGTAATTGCACCACCACACTCTAGCCT |
| | GGGTAACAGAGCAAGGTCCCATCTCCTAAAAAAAAAAGAAAGGAAAATAAAA |
| | AGAAAATAAACTATTCTCCATAATAATGTAGACAGCAATCCTCACTGTGAAC |
| | CAGAAGGAACCTCGGCAAATTTTTTAGACATCAATGGGATTTCACTATCAGC |
| | TGAGAGTGTTCCCTTTTTAGCATGGCAAGCTGTTTCCTGAAGCAATAGAGAG |
| | AAGCAAGACCAAGGAAAAATCTAGAAAGAGCCTCTCTGTAGAAAAGCAGAGC |
| | AATGATCTCTAATCACAATGCTATCAAATATTCCAGGCTAAATTTTCCTTTA |
| | TAGCATTAAAATTTTCCTCACATCCACAAGATTCCAATAGTTTTCTTAATGC |
| | CATAGCCTGGTGTCTATTCTGCCTTGTGGATTCCCATAATGCAAAATGCCAT |
| | TAAAAAAGGAACAGACCATGAGAAGTGGGCCTCCGAAGCACATGAAGCTTGG |
| | TATCATCAGAAAGATAAGGGGCAACAGTCAGGAATAATTGTTGGGACATTTA |
| | ATAAGTCCCTGGAAATTCCTAGAAACATAATTTTTTTTTGAGTCTAAGATGC |
| | TATCATTTTAAGGTGCACCATTATTTTATTTGCTACAATGTAGAAAACAATA |
| | ACACTGCCAATT |
| 2023 | TGATTAGGTAAAATATCAGAGACACAAATCAGGTTAAATTGATTTTTTATTG |
| | TAATTACATTTAAAATTTTAGAATTCATCAGTAGGTATGAACAAACATATAC |
| | ATACATATATATAATTTATATTATAAGTTTATTATTTATACTATACATTATA |
| | AAAATAACTGAGAGATAAACTTTCGTTTATCCTTAATGCTAAAATAATTCAT |
| | TTACCTTGGAGAGATCAGAACTCTGTCCATTTCCCCTACATAAAAACTAGAG |
| | AGTACTATTGCTTTCTCTTTCTCGGGCTTACTCTGGTCTCATAGAATATGCA |
| | TTTTCATTTTTTTTCAACAGAATATCCGTGGATAGCTAAAATTTCTGCTTCC |
| | TTTGTCAACATTTGTATTTCCCCAGTGGACATTTCTGCAAAATTTATTTTCA |
| | TTTCTTTGTTACCAGAGAAACTCTGTTGGTCAAGTTCAATAGCATCCTCAGC |
| | ATAATTTCAGAAGGAAATTACAGGGAGCAATTGAAGTCCATCACTTTCTTGG |
| | AGGGGAAATATTAACACCCTCACCTCTTGCTCCCAATATTAGGTGGTAGGCA |
| | GGAGTGAGTTACTCATTTTCTGAAGGAGCAGTAACTCTTTGGACCCCTCGAG |
| | TCACTTGGTAAATAAACTCTAGCACTGCCCCGAAGAGTGCCTCAGAGATTTC |
| | AAGGAATAAATGCTTTAAAGGTAGGAAAATGCTAAGAAACACCATCATATAA |
| | GTGAGTTATTTCCAATTTTATTTTAAATACAGCCATATATTATTACATACAG |
| | CCACACATTATTAAATAATGTATTAATACATTATTATTAAATACAGCCATAT |
| | ATATGTATATATGTGTGTGTGTATATATATACATATATATGTAAGTATGTAG |
| | CTGCTATACCCTCCTGAAGCAATGAATGTAGCTGCTATACCCTCCAGAAGCA |
| | ATGATACCCTCCAGAGGTGATAACAGATACAAGTAACAACCACACTCTCTGG |
| | TTTTGACAACCA |
| 2024 | CAGAGAGCTTCCAAGGCATTATCCCATCCAAAGGGTAAAGAGGCTGGGATAT |
| | TTATCGACTAGCTCCCATTCTTCACTGGCTGTAACTTGTCCACGTCTCACAG |
| | CTGTAACTCCCTTGTATTCCCTACCTATCTGGTGTGAGGACCAAGCTTGTGT |
| | CTGTGGATAGAGAAAGCCCTAAAGCAGAAAGTCTAGGTGCTTGCACAAAAAG |
| | ATCATCTGCACAGAATGATGATCAAGAGATGTGAGTGGGGCACCACAACATT |
| | TACCTCAGGAATCTCTGTTCAGGACTCAGCTTTGGTCTCAAACCTTGGGAAG |
| | CTTATACACTGAGGCAGTGTTAGGATCTCTTTTCTCTGCCTTCCTGTGCTTT |
| | TAAGTGTATTTCACTGTTTTTGATCCCTTGTCTGCCCCTTATATTTGACTAT |
| | CAGGCTCTTGAAGGTCTATTACACTTACTCATTGTTTTTACCCCCTGTTCCT |
| | ATCTCAGTGCCCAACACAGAGCTGACAGTTAATATATGTTGGTTGGATGCAT |
| | GTGTGGGTATCTTATCTTTTTATCCTTTAAAAGACCTCACACGTAGATGAAA |
| | ATTTTAAAATCATTAATTCAATCATCAATTCAATTCAATCATCTTTTTATCC |
| | TTTAAAAGACCTCACACATTGATGAAAATTTTAAAATCATTAATTCAATTGA |
| | AGAGGCCTTGTGATTGACATGAGTATAAATTGGACCATTATTAACTTCAAAC |
| | TAATTCTACTATGCCAGAAACCATGCCTGAAGTATTAAAACATCACGTTAAA |
| | AAACAAAAGACAAAAAAAAAACTTATCTAAAAAATTACATTAAATAAAATAG |
| | ACCAAAGGTAAATCTTACTCAAGTTTTCAGGAAAAAAAAATTGTTTTCTATA |
| | CTCTTTTCTCACCTATTCTTCCTTGTCACAGAGAAGCAATTATTATATTAGA |
| | CTTTCCTTTTTCAATGTGTAGATGACATCATATGATTTAAATTTTTTATGTA |
| | TTTCTCTTGCAA |
| 2025 | ATCAGCAGCAGAGGCTGCAGAACAGCGGATATTAGTGAAAAGCAAATGTTGC |
| | TGTCTGATCGTTCCTGTGGAAGTTTTGTCTCAGAGGAGTACCCGGCCGTGTG |
| | AGGTGTCAGTCTGCCCCTACTCGGGGGTGCCTCCCAGTTAGGCTACTCAGGG |
| | GTCAGGGACCCACTTGAGGAGGCAGTCTGTCTGTTCTCAGATCTCAAGCTGT |
| | GTGCTGGGAGAACCACTACTCTCTTCAAAGCTGTCAGACAGGGACATTTAAG |
| | TCTGCAGAGGTTTCTGCTGCCTTTTGTTGGGCTATGCCCTGCCCCCAGAGGT |
| | GGAGTCTACAGAGACAGGCAGGCCTTGAGCTGCAGTGGGCTCCACCCAGTTC |
| | GAGCTTCCTGGCTGCTTTGTTTACCTACAATGGTGGGCTCCCCTCCCCCAGC |
| | CTTGCTGCTGCCTTGCAGTTTGATCTCAGACTGCTGTGCTAGCAATGAGCGA |
| | GGCTCCATGGGCGTAGGACCCTCCGAGCCAGGTGGGATACAATCTTCTAGTT |
| | TGCTGTTTGCTAGGACCATTGGAAAAGCACAGTATTAGGGTGGGAGTGACCC |
| | GATTTTCCAGGTGCTGTCTGTCACCCCTTTCCTTGGCTAGGAAAGGGAATTC |
| | CCTGACCCCTTGCGCTTCCTGGGTGAGGTGATGCCTTGCCCTGCTTCGGCTC |
| | ATGCTCAGTGCACTGCACCCACTGTCTTGCACCCACTGTCCGACAATCCCCA |
| | GTGTGATGAACCCGGTACCTCAGTTGGAAATGCAGAAATCATTCATCTTCTG |
| | AGTCACTCACGCTGGGAGCTGTAGACTGGAGCTGTTCCTATTCGGCCATCTA |
| | CATGTTCTTTCTTCCCTCATCATCACTTCTTTACTTCTTTTATTTCACTTCT |
| | GGCTTTCTGTCCTCCCACGCTGAGGAAGACTGATTTGGTGGACATGTATTTA |
| | TTCTGCTGAGTACCAGTTGATGTGGAAGTAGTTGTTTTATAGTCAACATGTT |
| | TTTATGACTAAT |
| 2026 | GAGTGATGTCTAATCACAATCTGTGATAGGTATTTGCTTTAAGGTGCATCTA |
| | ATAACATGACAGTGATTTTCATCTCATATAACCTTCATTAACTCTGGTTCCC |
| | TGCTAAGATAAAGCCTTCCCTATAAGCCAACTGAGAATACTGTAGTCAGAAT |
| | TTACAGGTACTTCCCATTGTGGTTGTTCACCTTATTTGTGCCAGTTTTTCTT |
| | CTTCTTTATTCATACCTTTTGCCATGTGAATTTGCATTTCTTCTGGGTTGGA |
| | GTCAAGTATATATTTATCCTTTTTACCTTTGACTCTGAGGCTGGCCAAAGGA |
| | ATAAGGTGGATGTGACAAGGTACAATTTCTGAGCCTAGCCCTTAGAGGCCTT |
| | CCATGTTTCCACTTGTTCTCTTGCACTTGCGACGTTGCTGTCAAAAGAACAT |
| | GCAATGGCTAGCTAGCAGCCTGTGCACCTGCAGTGAGAACCAGAGCCACCCA |
| | GTTGCTGCAGCCTGAGACCAAGCTGCTCAGCTAAGCATAGCTTAGATCACCA |
| | TTGAGTTCTGAGGTGGTTTGTCATACAGCAATGGCAATCAGATATATCCACA |
| | CAAATATAATTTTAGTTTATATTTTTGTTACTGCAGTTCTCATCTTATTCTG |
| | AGGATACGTGACAAAATAATTCTTTCAAAAATATTGATGCTGTGCCAGATTA |
| | CTATTTTGAATGAATTATTAGACAAATACTTCATATGTATCTTATTATGTGG |
| | GTTTACACATTATTTATCTTATTGATTTAACTTCAAAACTAAACTTTAGTTT |
| | AGCTCTTGGGCCCTATCTGGGAAAGGGTCATCTTTTAATCACCATTAAATCA |
| | CTGAAGTCATCAGTTTATTCAAAGTACTCTGCACAAAATTAGCATTCTTTAG |
| | TGGTTGTGAAATAAATAGACTTTAAACTTATCATTAATATTCCCAATGGTAC |
| | TATGGGGGAGGCAAAATTTTCTATCTTCTTAGTGGTTTTTTTTTTTTTGGCT |
| | AGGGCTAAGGAT |
| 2027 | ACGCACCTGAGAAATGTGTTAAGGATTAAGATGCTAGTGCTAGATGTTTGAT |
| | TTTCTGAATCGAACCACTATTGGTGAGATCCAGAAGCTCAAAGACATGATAT |
| | ACCCACCTTCAAATAATGTTTATGTAGGTAATCTATTCTCAGGATTTATAGA |
| | CACTGCTGTTAAGACCTATTGTCATTGGGGTAAAAAAAAATCCTTATTATAT |
| | TATACAAATTATTATATACTATTATATTATAGAAATTATATTTCTATTAAAT |
| | AGCTTGTGTAGAAAGTAACCATATATAGTTAGAAAAACACTGATCTCAAGAA |
| | CAGGATTTTAGATTTGACTCTGACAATTTCTGTTCGGTCTTGTATAAATGTA |
| | TCAATTTAGATTTAGGGCTTTATTTTCTAATCCATAAAATGTGTAGCATACT |
| | TCTGCTAGCTATACATTTACTGAAGTTATTATTTTAAACTATTTTTATTTTC |
| | ATTTTTTTGTTTTGAGTTATAATCATAATTAATGGATTCAAGTGACAGAGAA |
| | AAGAAAGTAATTAGTCATCTTTTTTCAGAATACAGTCTTTGTTCTGAAGGTA |
| | TTTCGTATGAATCAAGTTTCAAATCTTCAGATAAATTTTCACCTTGCCAATG |
| | TGCTTTCTGCTCTAAATCATTCCTGAATTTTGCTATGATTTTTCTTTCTTAT |
| | AAAATCTTGACACTAAATTGTCAGGAGATATACATATATGTATATATGTAAA |
| | ATATATATATCATATATAAATATATATAAATTTTGAGTTAAAGTACTATTAC |
| | AGTATTCAATTCTACCAGTAATTCTAATAGTATGAAAATAAAGTCACCAGTT |
| | GAAGTAAGACCTACTGACACCTTCTATTATATTTCGATAATTCTATTTGAAA |
| | CTAATTATATAGTAGGACATTTTCATTGTTTTCAGTATTAACTGGCACTCAT |
| | GTAGATATTGCAGGCCAAATTTTACCTCTACCTTTTGGAATTTTCTGGGGTA |
| | GACTTGAGAATT |
| 2028 | TACATGTGTAAACAGTTTTAGCGTAGATTTCCTCGCACTTTTAAATTTTGGA |
| | TTCTTAATTTCCCTGTCCCCCCTGCCCCCCCCCCAAAAAAAACCTGCTAACG |
| | TTTAAACGAACACAGTTTGGGAAATCTGCGTTAAGTCCTTCGTGGGAGTGGG |
| | GTTGCTCAGCTCACAGTAGGCCACGAACCTGAATTTTCTCTTGTCTGCTGCC |
| | CCCTTTTGATAGATGGAGGGAAGAGCAGGCTTCCAGTGCAATGGACAGAAGA |
| | GGGAGCCTGCAAGTTGGTAACAGAGTCTATTAGGGAAAGAGAGAGTCACTTG |
| | AATCCTCAGAGCTGCTCCTGTCAACTGCTTTGTGCAGTTTTTGTGACTTATT |
| | AGCTGCTTGTTTGCACTCTATCTACGCCTGCCCAGGTGTGTTTGGGCCCTAG |
| | AGCGAAGGGAGCACAGGCGTTCATTTAGAAACTTATCCCTCCGTCCAAATAT |
| | TGGATGCTTACCATGTGCCTGGTGCAATGCAGGGTGATACAAAGAGGAAGAT |
| | AAGTGAGGCATTCTTATCGAAGGACCAGACACTCTTCCAGCCTGACTATATT |
| | CATTACACTCGTGCCTGACCTTTCTTTGACTCTAAGATTCTTCCTTTCTAAA |
| | TGTGAATCTTAAAGACTGAAGTCTTTGATCTAAGACTGCTTTCTTATCACAT |
| | CACATCCAACAACCAACTTTTCACAGCTTCCCAGATCCCAAATTCTGTTTAG |
| | CAAGGACACTTGGATTTTTTTGTTTTTTGTTATAAATGACCTCTTCAGGTTC |
| | ATATTTTCACTATGTCCAGAATTCTTATTTTATTCTGTTTTGTGCTGACATT |
| | GGAGGCAGAGTCTGTGTCACAGAATACACCACTAGGGGTTACCCTGGACATG |
| | GAAGGGTATTCACTCGGGGAAGAAATTTTAATGGAATTTTTAATATCTAGAG |
| | CTGTCATTATCCTGTGATGGTTCACAAGAAATGGAACACTTAAAAATTTCTA |
| | CAGAAAAAAAGG |
| 2029 | GCCACAAATTTGTTTTCTGTATCTGTAGATTTGCATTTTTTTCCGAACATCT |
| | CATATGAATAGAATCACAAAATTTGTGTATTTTGTGCCAAACTTCTTTCACT |
| | TAGCATACTGATTTCAAAATTGATCCAACTTATAGCATATATCAGTACTTTA |
| | TTCCTTTTTAGGGCAAAGAAATCTTCCATTACACGGATACCCCACATTTTAT |
| | TTCTCTACCCATCGCTTGCTGGGCATGAGTTGTTTGTGACAAATATTCATAT |
| | ACATATTCTTGTGTGGACATATGTTTTCGCTTCTCTTGGGTATATATCTAGG |
| | AGTAGGATTGCTGGGTCATATGGTAAGTCTCTATTTAATGGTTTAGACTCAG |
| | TACTTTGTTTTCTGCCTTTCCACAGCTCAGTTTCATAAAGAGGCAGGAGCCT |
| | TTTGTTCAGGGCTCCTTGGCAGTAAGGTAATTTCTTCTTCTGCATTGTATCC |
| | AGCTGACCCTTGCTCAGTGCTGTTCTTTGGGGGAAAGATGGAATGCTGGGAA |
| | GCCAGCACCTCTTATTCCTTCTAGCTAACACTTTTACAGTGACGGATATAAT |
| | AGATATCTTCAACTAGTATTGTTGAATTATCTCCCTGATGCTGTCCAATTTT |
| | GCTTCATATATTTTGGGGCTCTGTTATTAGGTATGCATATATAGTCATTATT |
| | GTTATATCTTTGTGGTGGTGTGGCCTTTTTATTATTTTAGCACTTTTATATC |
| | TTTACCTCTAATAACGTTTTTAAAAATTGAACGTTGATTTTGTCTGATGTTA |
| | GTACAACCACTTCAGCTTCTTTGTAGTTGCTGTTTGCATGACATATCTTTCT |
| | CCATTCTTTTACTTTCAATCTATTTGTATCTCTGGGTCTAAAATGTGTAGAT |
| | AGCACATAGTTGAATCTTTTAAAAAATACATTTTACAATCTCTGATTTTTAT |
| | TGGAATGTTTAATCCATCCACATTTAATGTTACGATTGATGGAGCTGGACTT |
| | ATTTCTGCCATA |
| 2030 | AACACAGAGCTAAAACCAAGTAAGAGGCGATTCTCCAAAAGCACTTCCTCAG |
| | CAAACAGCATATCTATTGTGTGTGGGTTCTTTAATTGGCTGAGAACTGAATT |
| | TCACCTTTGGCATTAAAGAGAAGTGTTTATTTTTACTGTCTTCACTGTTTTA |
| | ATGTTTAAACAAAATCTAAATACTGAGGTGAACTCTATCATAAAACAAGTGA |
| | AACGGCAACATAGGTTGATCCAGAAAGAAGCAAATTCCAGCATGGCGGGCAC |
| | TACATGTTTCAGCTCATCAGTTATCTGAATCTTATGGCTCTAAAGATGGATG |
| | GATGAGAATACATAGGCAGAAGCTTCCTGGTGAGGCTGGTATGATTCTGTTG |
| | TCCTATCTTCAACACTATCCTTCTACCTTCAGGGTTGCTGTTGTAGGTTTTA |
| | TTTCTTTGGCTTCTGTTGCCAGTAATGGAAAAGGACCACATGGAAGACTGTA |
| | TTTATGTACATCATGTCCAAACAGAATATCCTATAATAGTGAATCTTGGAAG |
| | AAAGCTTGAGAGATGTGGCCCAGCGCGGTGGCTCACACCTGTAATCCCAGCA |
| | CTTTGGGAGACTGAGGTGGGCTGATCACGAGGTCAGGAGTTCGAGACCAGTG |
| | TGACCAACATGGTGAAACCCCATCTCTACTAAAAAGACAAAAATTAGCCGGG |
| | CCTGGTGGTGTTGCACCCGTAATCCCAGCTACCCAGGAGGCTGAGGCAGGAG |
| | AATTGCTTGAACCCAGGAGGCAGAGGTTGCAGTGAGCCAAGATCGTACCACT |
| | GCACTCCAGCCCTCCAGCCTGGACAACAGAGCAAGACTCTGTCTCAAAAGAA |
| | AAAAAAAATACCAGTTTGAGAGATGTATGTGAGGACTGATTACCGAAAGCGA |
| | AAGGGTTTAGTACATCTCATGAGAACAGAGCAGTCACAAGTGATATAAACCA |
| | AACTCCCTTGGAAATTTGTAATCTATCAACTTCTTTATTTAAAGAGAATAGG |
| | AGGTTTACTGTG |
| 2031 | ACTCCCACTCCTACTAATTACAGCTTGTGTGTCCTTCAGTCATTCACTTCCC |
| | TTCACATGACCAGCCCAGCAGAAATGAACTACCAGGAACATGAGCTCAGAGC |
| | GATGGGCTGGCCACCTGCCAAGCACCTCTGAATGGAAAGAGCAGAATTTTGC |
| | ATTGCCTGCCATGCCACGTGGAGCAGGCCCTGGGTGGCTCTTTAGGGGATGG |
| | GTGTGGACTCCCACAACAAAACCAAGGGCCATATTCAAAGTTAAAAGCTCTG |
| | CCATAGATGGTATTTGTTGAGGCTGTGTGTGGTAGCTCATGCATGTATGCCC |
| | AACACTTTAGGAGGCTGAGGTGGGAAGATCACTTGAGGCTGGGAGTTCAAGT |
| | CTAGCCTAGGCAAGATAGTGAGATCCCTTCTCTAAAAAAGATAAAATATTAA |
| | CTGGGCATCATGGACGTGCCTGTAGCCCCAGCTACTGGGGAGGCTGAGGCAG |
| | GAGGATGGCTTGAGTCCAGGAGTTTGAGACTGCAGTGAGCTGTGATTGCACC |
| | ATTGCTCCCTAGCCCGGGTGACAGAACAAGACTTTTATTTCTTTAAAAAAAA |
| | AAAAAAAAAAGAAGGTGTTTACTGCAGTTGCTTTATTAAAAAAAAAGTAAAT |
| | GAATGTTCTGACTGTTCTACTTTTGAAAATAAGTGGCAAGGAATTAGAACTG |
| | TATCTTTCAGCAACAAAATGTACACTGTGGTTCCATGTCACAGCCAGGAATG |
| | GAGTCAGATGTCTCAGACCAGAATCACAGCTCTGCCACCTCCTGTGACATGG |
| | ACTTGCTAAGCTACCTTGACTCTCTGGAGCTCACTATGCCCATCAATAACAA |
| | GAAATAAATAAATCCGTCCTGTAAGGTTGTCAGGAGAAACAAATGAGGCACT |
| | ATATGTGGAAGTTCCTGGAATAGTGACCAGCACAGAGGACGTCTCAAAGAAA |
| | GATTTGCTGAACCCCAAAAGACAGGAGGACTGGAGGAACAACAAAGAGACAG |
| | GAAAGCTAGCAT |
| 2032 | AATTCATAGCCCAGCCAAGGAACTTAGAAGAGTAGAGGGAAGTCATTTTTCA |
| | CTCCCCTACAAGAACATTCTGCTGTAAAGAGGAGCTAGAAATAATTTTTGTT |
| | TTAAATTCAACCAAACATAGGGATAATTCTGAAATTTGGAACCAAAAGAATT |
| | ATAAGTACACTACTGGTGAATTTGTGCTTATCTGAAATCTACACATGTAGCT |
| | GTCTTTATGTATCTCTGTATATCGATGTTTTTCTATATATATAATCAGTGAA |
| | GTAAGATATCTAGTCATTCATTTACTCACCAAGTGATTGCAGTGGGGTGACA |
| | GGGACAGTGGGGGGTGTGGTGGCGGGTTGCCAGAGCATGAGGAGTATGCAAT |
| | AGAATCTAAGAAATCATACCTACCTGGCCAGGCACAGTTGCTCATGCCTGTA |
| | ATCCCAGCACTTTGGGAGGCAGAGGCAGGCGGATCACTTGAGGTCAGGAGTT |
| | CCAGACCAGCCTGGCCAACATGGTGAAATCCCATCTCTACTAAAAATACAAA |
| | AAATACAAAAAATTAGCTGGGTGTGGTGGCACATGCCTGTAATCCTGGCTAC |
| | TCTGGAGGCTGAGGCAGGAGAATGGCTTGAACCTGGGAGGCAGAGGCTGCAG |
| | TGAGCTGAAATTGTACTACTGCACTCCAGCCTGGGTGACAGAGTGAGACTCC |
| | ATCTCAAAAAAAAAAAAAAAAAAAAAAAATCAGACCTGCCTTCCATGAGCTC |
| | ATGGTATACTTGAATCTCCATAGGCTAGTTATTCAGGAGGGTATGTAATGTA |
| | ACTCAACAATGCACAATTACTTAAATTCGCTCAGGAGAATTACCTCATTTTG |
| | CCCAACTTGTTACTGTGAAAAAAAAAAAAGAAAGAAAATTTCAGGACCTTCC |
| | AAATTTATTATGCCAAAGGGAAAAGTCAAGCCCTGGAAACCAAGTCATGTAA |
| | CACGGCTGTTTTTCTTCTCTGGTGCATGACTGTTGCTTCCTGATCTTTTTGT |
| | TGATGTTATACA |
| 2033 | CATATAAATTAAATATTTATGTTATATTGAAGGAATACTTTTAGACTTGTTT |
| | AAACACAAATCTTTAAAAATTACATATCACTCTTGCATGTACATAAAAAATG |
| | AAAATATAGGCAATTAAATTAAGAGAGGTCTACAGTGTCTTTACATCAAGTC |
| | TGACTCTACTGAGTCCCTTTTTGACTCAGAGTCATTAATATATTGTTTTTTT |
| | CCAGTAATAATGTAGTGATGCAGCCTGTCTTCAAAGACTGCTCTACTATTGA |
| | CTCAGATTTTCTCCCAAGCCATTGATACTAGTTTTGAAGCTGATGCTTTTTA |
| | AATCTTGCTGTCAGACTTACGGGAAGGTTTTCATACAACAGGGCTCATATTC |
| | TTTCCTCAAATTATCCTTACATGTAAATGTTCAGAATGTCGAGATGATACAT |
| | AGGCCAGTTATGCCACTGTGAATATCTACCAAGGTCACATGTGTAATGAACA |
| | AAGACAGCTATTTCTGCTGCTGGCTGGCAGTGATTTGCAAGATTTTGTTGAC |
| | TGTAGGACATATCCTACTTCAATGATGTTAAAATGTGAACAAATATGCACTT |
| | CAGACTTTGTAAAATGTAGCACAGCACTTACAGAGCACACTAGGCTTCTGGC |
| | ACTCGCATAAAATGAAGACTTGGAGTTTTAGCTGAGTACTAAAGGAGGACCA |
| | TCCTCCCACCGAAGGATGAAGAATTTAAGGATATGTAAGTTGAGCTGTACTT |
| | ATGTTCATCTGTGATTTTTACAAGTCACTTATTGCTACATGTATCCTTTAAA |
| | TATGCGTTGTCCTTCCTCCTAAAATGGTTTCACCATAATAAGTGAAATGTCA |
| | GCTTGTCACATTAAATTATAAATTATAAATTACCATCACCTTAGTCCTCTAC |
| | ATATCCTTCAACTTCATTATGACACTGTCCTTCAGAGATAAGGAACAGAAAG |
| | GCTTTAATGAAAACTTCAGCTAATGTAATAATTAGGGAAGGATGAGCTAATT |
| | AAGAAACATACA |
| 2034 | CAAAGTCTCCCTAGAGGGCAAAATTGTCCCCATTGAAGACCACTGGGTTAGA |
| | TAGAAACTTACATCTCACACATGGAGAGTCCAGGCTGGCATGGTCGCTCTGC |
| | TGTGCACTGGGAGCCCAGGTTCCTCCTCGCTTTGCAAATTGTACAAGCTGCC |
| | CTCATCACCTGGATGCCTACATCTCACTTAAGAGTCTCAGTTCTAGGAGGGC |
| | ACAGACAATGGTGTACTGGTAAACAGACTCTGTTAAAAAAAAAAAAAAAAAA |
| | AACCAACACAATCAGGAACATTTTTTAAAAGCCCAGATTTGTAGTGTTTGCA |
| | GATTCTTATGTTTTAAATACTCCTGCCATGGCTGATGTGAAACTACCAACAG |
| | TTTAACAACTGGCTTACTAAATTTCTGAATATTTACCATTTGTCCCTTGTAA |
| | GACAGTATTAGTGGGCTGCAGTATATCAACAGAGAAAGGGAAGGAAAAGATA |
| | CAACCTTTTGTTGAAGGACAAAATGACATTTCACTTTTCTTCAGCCCCACTG |
| | GCCAAAACTTAGTCCCATGTTCACCTTAGCTGCAGGGGAGGCTGAAATGCAG |
| | TGTTTATTCTAAACAACCATGTATCCAGCCACAATACCAGGGGAATTTATCA |
| | CCAAGAGAAAGAGAGAGAGAATATCTAGTGCTTGAAAATTATCAGTCTCTGC |
| | CACAATTTTATTTAAAAAATAACCAGAAAAATGAGAGTGAATTTTATCTGAG |
| | AGGATCTTAGAAATCTCAGCATCGAGAAGGTAATAAATAAAGAGAGATAAGT |
| | CACAGACTTCCTGCGACAGTCAAGAATTCCCCATGCAGATGACACCCCAGGA |
| | GATGCCGGGTGATTGTTCTTACAATTTCTTCAGTTGAAGGTAAATGTGGCAC |
| | TAGCCATTTATTCTTTTAGCTCACGTTGTTTGAAGTGCATCGCCTATGTACT |
| | TCACCCTTTGGACTCACTAGAAAACAAAGAGAATTTTGGAATTAGAAGAGGC |
| | TTAATAATGTTA |
| 2035 | AAATATAAATAAAACATTTCTTTTGGAAATTTTATAATTCAAGCTAATTTAA |
| | AATTATGTAAACCTCTATCTTTCATGTAATCTTCTTCCTTCTTTTAAAACAA |
| | CATTTTTTTGGTGGTCATCTGTTCGGGAGAAAATGAAATTTTCTGTGGATAA |
| | GCAGATATTCTTCACGGAGAAAGCTAACATTCTGCATTCCTCTATTTTAAAA |
| | GTGGAAAACATAGTCCTGTTATTTGTATTTAGATGTATTTCTCACCAAAGAG |
| | TGCCAGGCTGGATTACAGAAGATCTATATTCTGATCTTGTCCTTTTTCTTTG |
| | CAAGCCTGAGGAATTGTCCAGACACAGAATTCCCTAGATCCCCAGATTTCTC |
| | ACCTATAATATGAAGGGTTGAAAGAGAGGTCTCAATCGGCTTTGAATTTTCT |
| | GTTCTATACTTCTGCACCACCACTGTAGCACTGACAATTGCATGAAAATATT |
| | AAGCTCTATTATGTTTTCAGTACTATCCTTAGCTTCTTTAAAAAATTAGTCT |
| | AGCTGTGTTTGTAAATAAATGATGTCACTGGAAAAATGGTTTCATACCATTG |
| | TTGTCAATAGTTGAATGTGGCTTGCCCTCAGGAACAATGCATTCTTCAATAA |
| | TATGGAGGATGGAAGGTGTATAAGGACTCAGATAGCTATTATTCTCATTTGC |
| | CCATGATCCTTTCATATCCCCGCCTCTGGTTTAGCATTCTCTTTCTTCCAGG |
| | GGAATTTCTCCCCCATTCCATGCATTCTAGTAGAATTTTTTATCACAGTAGA |
| | TTGTCCTGCCCTGCCACAGAAATGGGCATTTGACACAGTGGCCACAAAGATT |
| | GGTCTAAGCAGTAGGCCTGTGACCCAAGGTAGGCCAATTAGAGTTTTCTGTA |
| | GAATTTTTTAGATTCAAAGTGTATGTGTGTGGGGGGGATGACTCTTCTTGAA |
| | TTTTATATTAGGATGCATGCCAGAAATTGTTGAAAGGTCTTTAATGTACCAT |
| | GTACAGGAAGCT |
| 2036 | CACCTATAAGAGGAAATATACTTATGTCTAGGTGGACTCCAATGTGTCTGTT |
| | TACTGATACTTATTTATTCATTATTTTCAAGTAAAATGTAGAAGTGAATAAC |
| | TTAAGAGAATAACTATTTTTATGAGAGAAAAATACCCACTTTCTTTTTTATT |
| | ACTTTGTTCCTCTAGAGGTTCATGAATAATATATTGAACATGTGAGGAGTGA |
| | GGCCTGTCTAGCTCTTTTCCTAACATCTTCCACTCCTGTGGCCTCTTATTAG |
| | GTACCTTTCTCAGTGAAGATATACAATAAGAATTTTGCATGCTTATTGGGAA |
| | TTTATCTGTGAAAAATCACTCAAATGTCATTAAGTCTTTTCTGATAAACCTT |
| | AATCATCCAACAACCAGAGTTTTTCTTAAAATAGCTGTTGCTCTAGAAGAAT |
| | ACCATAGAATGAAGTTGCTTCCTAGCATGGCAGTCAAGGATCCTGGTTCCAA |
| | GTATGAGCTCTGAAGAAGATAGACTATGTTCACCGCTTACTATAGCTGAGTG |
| | CCCTTGGACAATTCATTTAAACTGCCCCTAATTTTCTTCCATCATCTGTAAA |
| | ATGAATGTAATAATAGCTCTTAATGAGTATTAAATTAGATAATAAGGGCACT |
| | GGCATTTATTAAGAACTTAATAAATGTTAGCTTTTGTTATTTCACATTTTTC |
| | CTTGATCACTCCTACCAGGAATAAAATTCTGGGAGGGTATAAGTAGGTAGTG |
| | AAGTGCTAACTGGTCTGGTTAATTGTTAGAGTTCTGTTAAAAAAAAGTTATT |
| | TGAAAAAAGTATTTTGGAGCTAGGATCTAATTTATTAATATATCTGGATTTT |
| | CTTTTTCAATTTTGGTGTCCATTATTCACATAAGTAATTGTGGTTTTGCTAT |
| | ATTTTTTCCTCCTGAAAAATTATGGCTATACAACTAACTTTATTGTATACTG |
| | AATTTTGGAATTTTTTAGGATTTGATGTTCTTACTGGGGAGAGGATTTTGAA |
| | TTATTTAACCAC |
| 2037 | AACAAGAGGAAAGCATACAAATTTATTTAATACATGTTTTATGTGGCACAGG |
| | AGCCCTCATAAAGTAATAAAAAATCCCCAAACACAGTTAGAGCTGAACATTT |
| | ATATACTAATCTGGACAAAACATTTATATACTGCGTGGACAAAGAGCAGTAA |
| | ATTGTGAAAATGGAACAAGGCAAGGGGGCTTAGACTACAGTAGTTAATCATC |
| | AAGAAGTGACAAAAAAAAATAAGGGTTAGTTAATAAGATTTGTTTAAGCAGA |
| | TTTCTCCCAGCTTTAGCTCTCTGTCTCTGGTGATCAGAATGCACTCCTTCCT |
| | TCAGACTCAGTGAGCACATATTCCACACGGAAGATTTCTTCCCTAGCTTTTA |
| | GGAAATCCAGAGAACCCTTTTTGTATCTGTTGTTTTTTTTTTTTTTTAAATG |
| | TCTTGTCTTTAACTCAAAACAATTTATGTGCCAGGATGACATATCTTTGGAT |
| | AATGTGTTCTGAACTCCTTCAGTACATACGTATATAAATTAAAGCAAATATT |
| | TTTTATGATAAGCTGGCATAATAGTTTCATAATTTAATCACTGATTTAAAAA |
| | TTTAATTAAAATTATTTTTTAATATTTTGTGTAATAATTTTTGAGGAGTATC |
| | TTTTGTGCTTAATGAGTGGCAGATGACACCCATGTTCTTAGCAGCATCATTC |
| | ACAATAGCTAAAAGATAGGAACAACTGCGTATTGATGGATGAATGGATAAGC |
| | AAAATGAGGTATATACATATAAGGGAATATTCTTCATCCTTAAAAAGGAAGG |
| | AAATTCTGACATATGCTACAACAAGGTTGAACCTCTAAGGACATTATGCTAA |
| | ATGAAATAAACCAGTCTCAAAAAGACAAATACTATGTGATTCCAGATACATA |
| | AGGCACCTAGAGACAAACTGATAGAGACAGAAAGTAGAATGAGTGATTACCA |
| | GGGGTTGTGAGAGGAAAAAAGAGAGGGTTGTTTGATACAGAGTTTCAGTTTT |
| | GCAAGATAAAAG |
| 2038 | AACAGGAGAAAAGCGTACAAGTTTATTAAATAGAAGTTTTGCAGCCGGGCGC |
| | GCTGGCTCACGCTTGTAATCCTGGCACTTTGGGAGGCCGAGGCGGGCAGATC |
| | ACGAGGTCAGGAGATCGAGACCACGGTGAAACCCCGTCTCTACTAAAAATAC |
| | AACAAATTAGCCAGGCGTGGTAGCGAGGCAGGAGAATGGTGTGAACCCGGGA |
| | GGCGGAGCTTGCCTCTGCACTCCAGATCATGCCACTGCACTCCAGCCTGGGT |
| | GACAGACCAAGACTCCGTCTCAAAAAAAAAAAAAAAAAAAAAAAAAAAAAAG |
| | AAGTTTTGCATGACATGGGAACCCTCATAAAAAAAGTGAAGTCCCAAAAAAG |
| | TGGCAAAATCTAAATGCTTTTATATTATGTTGACGAAAGAGGGGCAATTGTG |
| | GAAAAGTAACTAAATTATGAGGGTTAGGCTAACAGAAGATAAAAATTATTTT |
| | AACAAGTTCTGTTTGTATAAAATTTTCTCAATTTCAGCTACCCATCCTTGAT |
| | GATTAGAATGTTGCATTCCTTCTGGTATACAAGGAACATCTTCCATATGGGG |
| | GTTTTATCTTCTGCTTTCAGAAAAAAAAAAATAACTCTGTGTGTGGTAGGAT |
| | GAAGGTGTCAGAACATTCTTCTTGCACCTGCTGGTTGCTATCTTTTTAAACT |
| | GCATTTGTTTCAAAACAATCCTTATGACAAAGGGGTGTATTTTGGGGTGGCA |
| | TATTCTGTCACCCGTCAATATCAAAGTGGTTTTTGAGTTTGTGCTCATCTTC |
| | TTTCCTTATTCTGTTCCTTGTAAGGTAAGACTAAATATAATGGAATTTGCCG |
| | TCACATGTCTCTTATATGTAGTGAGTTTTAACAGGCATTCCAGGAAATGCCA |
| | TATGGTCTTTTAGCTTGGAAATTATTTTAGAAAACGATAAAATCTTTAGTGT |
| | GAAGTTATTTCCCAGATATGTATCGCTAAAATTATCATTACAGGTGCTCTAG |
| | GTAATATGTTTG |
| 2039 | TTAGTACTTCCATCCCTTTCCTGGCTGCTCTAACTTTACAGGTACTTGTAAG |
| | TGGCAATTAAGCACTTTTTCCTAATTCCAGAGTCTTGCCCCACTTCAGAGCA |
| | ACATAGAGTGGCCTAGACAGGCTGAGGTACTTTGCCGCTTCAGTCATCATTA |
| | ATCTATGGTATTTACTGATGAGAAGTAAAGTGGTAGAAGAAAAAAAAATTTT |
| | CTGTTATCCTGGGCACTTGGAAATGAATGTATTCTCACAATCTGTTCTCAAA |
| | ACAACTTACTGATTCTGGGGTTCTGGAAGCTCTGATGTGCAGGTGAGCCTTT |
| | TAAATTCCTCACTGTTGGAGCTCCTATCTAGGACTCACTGGCTGGATGAAAA |
| | CGGTTCTTTTTATTGCTTTCTGAATGTCTGCTAGACAGGCGTAAGCAACACC |
| | TTATATCTGCCTTCTGAAAAAGGTAAAAGAACTGGGACCCATCCACCATGCT |
| | GGACAGCTCGGCAGTGGCAGTGGCCTCCCCCAGACCCTGTTCCGAGTGCTCC |
| | ACCAACAAACTCACCAGCAGTCAGAGTCTAGCCTCTCCCCAAACTTCACCTT |
| | CATCACAATTCATTTTAAGCCCTTCCACAACCCAATCAACTCTAGATCTACT |
| | TAATGGATAATAATTTGATCTCATGCAAACTGCACTTTCCTCTTCTCAGAAT |
| | GATCCTTCTACCCCTTAATTAAACATTTGAGAGTGAAAGAAGAGAAAATTCG |
| | GGTTCAAAGATTGGTAAGTCTAAGAAACCTAAGGAAAAGGAGTTAGTAAACA |
| | TGTTAATCAAAGAGTGAGCACTTTTCGGAAGCGCAACATTCAGATACCTTTC |
| | TTGATTGGATTCCAGAAGACTATTTCTGGGAAGAGGAGATTTGCATTTTTCT |
| | AAAGTCTTCTACCCACAGCCTAACCACCCTAGGGCTTTGAAATATTTTTTTT |
| | CTGATGTGCAGTCATAATTGAATAAATAAAATGATTCCTGATCATTTCTTCT |
| | CTTCAGCTTTAT |
| 2040 | TATTCCTGTATTTCTATTGTACTTTTTTGCATTAAGAAACATTTTCCAATGT |
| | AACATTTTAATAGATTTTTCACTATTTGTTGAGTTATTTTTGAGTGGTTGTA |
| | CTTGAGCTTGCCATCTATGTCTTAACTTCAGATTTGTACTAACTTAATTCCA |
| | GGGAGATATAGAAGCATTATTCCTACATAGCTCTATATCAACCCCCTTTTCC |
| | TGTGGTATTATTGTTATACAAGGTACACCATATATGTTACAAATACAATTAT |
| | TTATAGTTATAATTATTACTTTAAATATATCATTTATGTCTATTAAAGAAGC |
| | TGAGAGCAGAGAGGAGATAAAGTATATATTTATAGAATTTGTTATATTAAGC |
| | TTCTTATTTGTCATTCTGATTCTCTTTGTTCTGGTGGACTTGAGTAAATATG |
| | TGATGTTATTTCATTATGCACACACAGCTTTGCTCCTTGTCATTTTATTTAT |
| | GCTGTCTTTCTCAAGTATATTGCATTTAAATACATTATAGGACCAACAATTC |
| | AAATATATTTATGTTGTGTTATACAATTGCTTTTTAAAATCAGTTAAGATAG |
| | ATGGGATATGCACTGATAGTATGGTTTTTAAAATTATACTTTAAGTTCTGGG |
| | TTACATATGCAGAACATGCTGTTTGGTTACATAGGTATACACGTGCCATGGT |
| | GGTTTGCTGCACCCATCAACCCACCACCTACATTAGGTATTTCTCCTAATGT |
| | TATCTGTCCTCTGGCCTCCAACCCCCCGACATGCCCCAGTGTGTGATGTTCC |
| | CCTCCCTGTGTCCATGTGTTCTCATTGTTCAACTCCCACTTATGAGTGAGAA |
| | CATGTGGTGTTTGGTTTTCTGATCTTGTGATAGTTTCCTGAGAATGATGGTT |
| | TCCAGCTTCATCCATGTCCCTGAAAAAGATATGAACTCATCCTAGACAATAA |
| | TTCAAACACACACACACACACACACACACACACACACACACACACGCAAATG |
| | GCACTAGTATCT |
| 2041 | TCCAGAAAACATAACAATTCAGAACATATATTTAATCCCTCCTCAATCCAGA |
| | TCCTTGTTGAAACAATGAAAGAGTACAATATACTGCCATGAAAAGTACTGAG |
| | AAAAGTCTACAGATAGTGACATGGAAGAAAAGAAAAAATATTAAATAGATCA |
| | AACTAGTTATATAATTTGTATCTCATTTCTGTAAAATAAATTTAACATTTAT |
| | AAGTGTATTAGTTTGTTCTCACATTGCTATAATAAAATACCTGAGACTGGGT |
| | AATTAAAAAAAAAAACAGATTTAATTGGCACACAGTTCTATAGGCTGTACAG |
| | AGAAAACAGTGGCTTCTGCTTCTGGGGAGGTTTCAGGAAACTTCCAATCATG |
| | ATGGAAGCCGAAGGGGAAGCAGACACATCTTACGTGGCCAGAGCAGGAGCAC |
| | AAGTGTGAAGGGAAGTGTCTGTTCATATTCTTCACTCACTTTTTAATGGGGT |
| | TGTTTGTTTTTTTCTTAGAAATTTAAGTTCCTTGTAGATTCTGGATATTAGG |
| | CCTTTGTCAGATGGATAGATTGCAAAAATGTTCTCCCATTCTGCAGGTTGCC |
| | TGTTCACTTTGATGATAGTTTCTTTTGCTGAGCAGAAGCTCTTTAGTTTAAT |
| | TTTGCAGGGACATGGATGAAGCTGGAAACCATTATCTTCAGTAGACTAACTG |
| | TTAACAGGAACAGAAAACCAAAAACAAACAAAAGCATGAAGAGGGAAGTGTC |
| | ACCCACATGAGAACTCACTATTGTGATGACAACACCAAGGGGAATGGTGTTA |
| | AACCATGAGAACCGGCCCCCATGATCCAATCACTTCCCACCAGGCCCCACCT |
| | CCAATACTGGATATTACAATTCAACAAGAGATTTGGGCAGGAATACAGATCC |
| | AAACCATATCAGTAAATATAATAAATATATATTAATAAATATGTAAATATAT |
| | GTATGCAAGTTAACAAATGAACCAGTTGGTATGTAAGTATGTATATAAAGGA |
| | CCATAGCAGTTA |
| 2042 | CTGAATACTAGAGGAGCAAGTACAACAAATGGAAAATGGGATCAAGTATGAG |
| | TGAGAGTTGCTAAGATGCCTGGTAGGGATGCAAAGGGGTAGAGAGCCTGGGG |
| | AGAGAGGGTGAGGGAGGGAAGCACTGGTTTCTCAAGCAAAAGCTAAAATTTT |
| | TCTATTAAGATTTAACCTGATGCTACACTTTGGTGGTGCAGCAAGGGTCTCA |
| | AATGGTATAAAACTCAGGTGATCATGCTTTATGTCTGTCTCTAGAAAAATGC |
| | TCCAAAAATGATAAGTAGTGATAATCCGCAGTCTCGTTGCATAAAATCAGCC |
| | CCAGGTGAATGACTAAGCTCCATTTCCCTACCCCACCCTTATTACAATAACC |
| | TCGACACCAACTCTAGTCCGTGGGAAGATAAACTAATCGGAGTCGCCCCTCA |
| | AATCTTACAGCTGCTCACTCCCCTGCAGGGCAACGCCCAGGGACCAAGTTAG |
| | CCCCTTAAGCCTAGGCAAAAGAATCCCGCCCATAATCGAGAAGCGACTCGAC |
| | ATGGAGGCGATGACGAGATCACGCGAGGAGGAAAGGAGGGAGGGCTTCTTCC |
| | AGGCCCAGGGCGGTCCTTACAAGACGGGAGGCAGCAGAGAACTCCCATAAAG |
| | GTATTGCGGCACTCCCCTCCCCCTGCCCAGAAGGGTGCGGCCTTCTCTCCAC |
| | CTCCTCCACCGCAGCTCCCTCAGGATTGCAGCTCGCGCCGGTTTTTGGAGAA |
| | CAAGCGCCTCCCACCCACAAACCAGCCGGACCGACCCCCGCTCCTCCCCCAC |
| | CCCCACGAGTGCCTGTAGCAGGTCGGGCTTGTCTCGCCCTTCAGGCGGTGGG |
| | AACCCGGGGCGGAGCCGCGGCCGCCGCCATCCAGAAGTCTCGGCCGGCAGCC |
| | CGCCCCCGCCTCCAGCGCGCGCTTCCTGCCACGTTGCGCAGGGGCGCGGGGC |
| | CAGACACTGCGGCGCTCGGCCTCGGGGAGGACCGTACCAACGCCCGCCTCCC |
| | CGCCACCCCCGCGCCCCGCGCAGTGGTTTCGCTCATGTGAGACTCGAGCCAG |
| | TAGCA |
| 2043 | GCCCTGCCAGGACGGGGCTGGCTACTGGCCTTATCTCACAGGTAAAACTGAC |
| | GCACGGAGGAACAATATAAATTGGGGACTAGAAAGGTGAAGAGCCAAAGTTA |
| | GAACTCAGGACCAACTTATTCTGATTTTGTTTTTCCAAACTGCTTCTCCTCT |
| | TGGGAAGTGTAAGGAAGCTGCAGCACCAGGATCAGTGAAACGCACCAGACGG |
| | CCGCGTCAGAGCAGCTCAGGTTCTGGGAGAGGGTAGCGCAGGGTGGCCACTG |
| | AGAACCGGGCAGGTCACGCATCCCCCCCTTCCCTCCCACCCCCTGCCAAGCT |
| | CTCCCTCCCAGGATCCTCTCTGGCTCCATCGTAAGCAAACCTTAGAGGTTCT |
| | GGCAAGGAGAGAGATGGCTCCAGGAAATGGGGGTGTGTCACCAGATAAGGAA |
| | TCTGCCTAACAGGAGGTGGGGGTTAGACCCAATATCAGGAGACTAGGAAGGA |
| | GGAGGCCTAAGGATGGGGCTTTTCTGTCACCAATCCTGTCCCTAGTGGCCCC |
| | ACTGTGGGGTGGAGGGGACAGATAAAAGTACCCAGAACCAGAGCCACATTAA |
| | CCGGCCCTGGGAATATAAGGTGGTCCCAGCTCGGGGACACAGGATCCCTGGA |
| | GGCAGCAAACATGCTGTCCTGAAGTGGACATAGGGGCCCGGGTTGGAGGAAG |
| | AAGACTAGCTGAGCTCTCGGACCCCTGGAAGATGCCATGACAGGGGGCTGGA |
| | AGAGCTAGCACAGACTAGAGAGGTAAGGGGGGTAGGGGAGCTGCCCAAATGA |
| | AAGGAGTGAGAGGTGACCCGAATCCACAGGAGAACGGGGTGTCCAGGCAAAG |
| | AAAGCAAGAGGATGGAGAGGTGGCTAAAGCCAGGGAGACGGGGTACTTTGGG |
| | GTTGTCCAGAAAAACGGTGATGATGCAGGCCTACAAGAAGGGGAGGCGGGAC |
| | GCAAGGGAGACATCCGTCGGAGAAGGCCATCCTAAGAAACGAGAGATGGCAC |
| | AGGCCCCAGAAGGAGAAGGAAAAGGGAACCCA |

In certain cases, expression of an exogenous DNA, e.g., transgene, inserted in a target polynucleotide at or near a target nucleotide sequence may depend on cell type and differentiation stage, as one or more components of a target polynucleotide get activated during differentiation while others get silenced, which may or may not be correlated with rearrangements of the chromatin architecture reorganization during differentiation. To overcome this, in certain embodiments, additional to the exemplary characteristics described above, a suitable target polynucleotide comprising a target nucleotide sequence demonstrates suitable expression of an inserted exogenous DNA, e.g., transgene, throughout differentiation and clonal expansion.

### IV. PHARMACEUTICAL COMPOSITIONS

Provided herein is a composition (e.g., pharmaceutical composition) comprising a guide nucleic acid, an engineered, non-naturally occurring system, or a eukaryotic cell, such as a guide nucleic acid, an engineered, non-naturally occurring system, or a eukaryotic cell, disclosed herein. In certain embodiments, the composition comprises an RNP comprising a guide nucleic acid, such as a guide nucleic acid disclosed herein, and a Cas protein (e.g., Cas nuclease). In certain embodiments, the composition comprises a single guide nucleic acid, such as a single guide nucleic acid disclosed herein. In certain embodiments, the composition comprises an RNP comprising the single guide nucleic acid, and a Cas protein (e.g., Cas nuclease). In certain embodiments, the composition comprises an RNP comprising the targeter nucleic acid, the modulator nucleic acid, and a Cas protein (e.g., Cas nuclease). In certain embodiments, the composition comprises a complex of a targeter nucleic acid and a modulator nucleic acid, such as a complex of a targeter nucleic acid and a modulator nucleic acid disclosed herein. In certain embodiments, the composition comprises an RNP comprising the targeter nucleic acid, the modulator nucleic acid, and a Cas protein (e.g., Cas nuclease).

In certain embodiments provided herein is a method of producing a composition, the method comprising incubating a single guide nucleic acid, such as a single guide nucleic acid disclosed herein, with a Cas protein, thereby producing a complex of the single guide nucleic acid and the Cas protein (e.g., an RNP). In certain embodiments, the method further comprises purifying the complex (e.g., the RNP).

In certain embodiments, provided is a method of producing a composition, the method comprising incubating a targeter nucleic acid and a modulator nucleic acid, such as a targeter nucleic acid and a modulator nucleic acid disclosed herein, under suitable conditions, thereby producing a composition (e.g., pharmaceutical composition) comprising a complex of the targeter nucleic acid and the modulator nucleic acid. In certain embodiments, the method further comprises incubating the targeter nucleic acid and the modulator nucleic acid with a Cas protein (e.g., the Cas nuclease that the targeter nucleic acid and the modulator nucleic acid are capable of activating or a related Cas protein), thereby producing a complex of the targeter nucleic acid, the modulator nucleic acid, and the Cas protein (e.g., an RNP). In certain embodiments, the method further comprises purifying the complex (e.g., the RNP).

For therapeutic use, a guide nucleic acid, an engineered, non-naturally occurring system, a CRISPR expression system, or a cell comprising such system or modified by such system disclosed herein is combined with a pharmaceutically acceptable carrier. The term “pharmaceutically acceptable” as used herein can refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit-to-risk ratio.

The term “pharmaceutically acceptable carrier” as used herein includes buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers, and adjuvants, see, e.g., Martin, Remington's Pharmaceutical Sciences, 15th Ed., Mack Publ. Co., Easton, PA (1975). Pharmaceutically acceptable carriers include buffers, solvents, dispersion media, coatings, isotonic and absorption delaying agents, or the like, that are compatible with pharmaceutical administration. The use of such media and agents for pharmaceutically active substances is known in the art.

In certain embodiments, a pharmaceutical composition disclosed herein comprises a salt, e.g., NaCl, MgCl2, KCl, MgSO4, etc.; a buffering agent, e.g., a Tris buffer, N-(2-Hydroxyethyl) piperazine-N′-(2-ethanesulfonic acid) (HEPES), 2-(N-Morpholino) ethanesulfonic acid (MES), MES sodium salt, 3-(N-Morpholino) propanesulfonic acid (MOPS), N-tris [Hydroxymethyl] methyl-3-aminopropanesulfonic acid (TAPS), etc.; a solubilizing agent; a detergent, e.g., a non-ionic detergent such as Tween-20, etc.; a nuclease inhibitor; or the like. For example, in certain embodiments, a subject composition comprises a subject DNA-targeting RNA, e.g., gRNA, and a buffer for stabilizing nucleic acids.

In certain embodiments, a pharmaceutical composition may contain formulation materials for modifying, maintaining, or preserving, for example, the pH, osmolarity, viscosity, clarity, color, isotonicity, odor, sterility, stability, rate of dissolution or release, adsorption, or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) BIOENG. TRANSL. MED. 1:10-29). In certain embodiment, the pharmaceutical composition comprises an inorganic nanoparticle. Exemplary inorganic nanoparticles include, e.g., magnetic nanoparticles (e.g., Fe3MnO2) or silica. The outer surface of the nanoparticle can be conjugated with a positively charged polymer (e.g., polyethylenimine, polylysine, polyserine) which allows for attachment (e.g., conjugation or entrapment) of payload. In certain embodiment, the pharmaceutical composition comprises an organic nanoparticle (e.g., entrapment of the payload inside the nanoparticle). Exemplary organic nanoparticles include, e.g., SNALP liposomes that contain cationic lipids together with neutral helper lipids which are coated with polyethylene glycol (PEG) and protamine and nucleic acid complex coated with lipid coating. In certain embodiment, the pharmaceutical composition comprises a liposome, for example, a liposome disclosed in International (PCT) Application Publication No. WO 2015/148863.

In certain embodiments, the pharmaceutical composition comprises a targeting moiety to increase target cell binding or update of nanoparticles and liposomes. Exemplary targeting moieties include cell specific antigens, monoclonal antibodies, single chain antibodies, aptamers, polymers, sugars, and cell penetrating peptides. In certain embodiments, the pharmaceutical composition comprises a fusogenic or endosome-destabilizing peptide or polymer.

In certain embodiments, a pharmaceutical composition may contain a sustained-or controlled-delivery formulation. Techniques for formulating sustained-or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(−)-3-hydroxybutyric acid. Sustained release compositions may also include liposomes that can be prepared by any of several methods known in the art.

A pharmaceutical composition of the invention can be administered by a variety of methods known in the art. The route and/or mode of administration vary depending upon the desired results. Administration can be intravenous, intramuscular, intraperitoneal, or subcutaneous, or administered proximal to the site of the target. The pharmaceutically acceptable carrier should be suitable for intravenous, intramuscular, subcutaneous, parenteral, spinal, or epidermal administration (e.g., by injection or infusion). Depending on the route of administration, the active compound (e.g., the guide nucleic acid, engineered, non-naturally occurring system, or CRISPR expression system disclosed herein) may be coated in a material to protect the compound from the action of acids and other natural conditions that may inactivate the compound.

Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution. In certain embodiments, the pharmaceutical composition is lyophilized, and then reconstituted in buffered saline, at the time of administration.

Pharmaceutical compositions of the invention can be prepared in accordance with methods well known and routinely practiced in the art. Sec, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the guide nucleic acid, engineered, non-naturally occurring system, or CRISPR expression system disclosed herein is employed in the pharmaceutical compositions of the invention. The compositions disclosed herein are formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time, or the dose may be proportionally reduced or increased as indicated by the exigencies of the therapeutic situation. It is especially advantageous to formulate parenteral compositions in dosage unit form for case of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions disclosed herein employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

### V. THERAPEUTIC USES

Guide nucleic acids, engineered, non-naturally occurring systems, and the CRISPR expression systems, e.g., as disclosed herein, are useful for targeting, editing, and/or modifying the genomic DNA in a cell or organism. These guide nucleic acids and systems, as well as a cell comprising one of the systems or a cell whose genome has been modified by one of the systems, can be used to treat a disease or disorder in which modification of genetic or epigenetic information is desirable. Accordingly, provided herein is a method of treating a disease or disorder, the method comprising administering to a subject in need thereof a guide nucleic acid, a non-naturally occurring system, a CRISPR expression system, or a cell disclosed herein.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The terms “treatment”, “treating”, “treat”, “treated”, or the like, as used herein, can refer to obtaining a desired pharmacologic and/or physiologic effect. The effect may be therapeutic in terms of a partial or complete cure for a disease and/or adverse effect attributable to the disease or delaying the disease progression. “Treatment”, as used herein, covers any treatment of a disease in a mammal, e.g., in a human, and includes: (a) inhibiting the disease, i.e., arresting its development; and (b) relieving the disease, i.e., causing regression of the disease. It is understood that a disease or disorder may be identified by genetic methods and treated prior to manifestation of any medical symptom.

For minimization of toxicity and off-target effect, it can be important to control the concentration of the CRISPR-Cas system delivered. Optimal concentrations can be determined by testing different concentrations in a cellular, tissue, or non-human eukaryote animal model and using deep sequencing to analyze the extent of modification at potential off-target genomic loci. The concentration that gives the highest level of on-target modification while minimizing the level of off-target modification is generally selected for ex vivo or in vivo delivery.

It is understood that the guide nucleic acid, the engineered, non-naturally occurring system, and the CRISPR expression system disclosed herein can be used to treat any suitable disease or disorder that can be improved by the system in a cell.

For therapeutic purposes, certain methods disclosed herein is particularly suitable for editing or modifying a proliferating cell, such as a stem cell (e.g., a hematopoietic stem cell), a progenitor cell (e.g., a hematopoietic progenitor cell or a lymphoid progenitor cell), or a memory cell (e.g., a memory T cell). Given that such cell is delivered to a subject and will proliferate in vivo, tolerance to off-target events is low. Prior to delivery, however, it is possible to assess the on-target and off-target events, thereby selecting one or more colonies that have the desired edit or modification and lack any undesired edit or modification. Therefore, lower editing or modifying efficiency can be tolerated for such cell. The engineered, non-naturally occurring system of the present invention has the advantage of increasing or decreasing the efficiency of nucleic acid cleavage by, for example, adjusting the hybridization of dual guide nucleic acids. As a result, it can be used to minimize off-target events when creating genetically engineered proliferating cells.

In certain embodiments, the guide nucleic acid, the engineered, non-naturally occurring system, and/or the CRISPR expression system disclosed herein can be used to engineer an immune cell. Immune cells include but are not limited to lymphocytes (e.g., B lymphocytes or B cells, T lymphocytes or T cells, and natural killer cells), myeloid cells (e.g., monocytes, macrophages, eosinophils, mast cells, basophils, and granulocytes), and the stem and progenitor cells that can differentiate into these cell types (e.g., hematopoietic stem cells, hematopoietic progenitor cells, and lymphoid progenitor cells). The cells can include autologous cells derived from a subject to be treated, or alternatively allogenic cells derived from a donor.

In certain embodiments, the immune cell is a T cell, which can be, for example, a cultured T cell, a primary T cell, a T cell from a cultured T cell line (e.g., Jurkat, SupTi), or a T cell obtained from a mammal, for example, from a subject to be treated. If obtained from a mammal, the T cell can be obtained from numerous sources, including but not limited to blood, bone marrow, lymph node, the thymus, or other tissues or fluids. T cells can also be enriched or purified. The T cell can be any type of T cell and can be of any developmental stage, including but not limited to, CD4⁺/CD8⁺ double positive T cells, CD4⁺ helper T cells (e.g., Th1 and Th2 cells), CD8⁺ T cells (e.g., cytotoxic T cells), tumor infiltrating lymphocytes (TILs), memory T cells (e.g., central memory T cells and effector memory T cells), regulatory T cells, naive T cells, or the like.

In certain embodiments, an immune cell, e.g., a T cell, is engineered to express an exogenous gene. For example, in certain embodiments, an engineered CRISPR system disclosed herein may catalyze DNA cleavage at the gene locus, allowing for site-specific integration of the exogenous gene at the gene locus by HDR.

In certain embodiments, an immune cell, e.g., a T cell, is engineered to express a chimeric antigen receptor (CAR), i.e., the T cell comprises an exogenous nucleotide sequence encoding a CAR. As used herein, the term “chimeric antigen receptor” or “CAR” includes any artificial receptor including an antigen-specific binding moiety and one or more signaling chains derived from an immune receptor. CARs can comprise a single chain fragment variable (scFv) of an antibody specific for an antigen coupled via hinge and transmembrane regions to cytoplasmic domains of T cell signaling molecules, e.g., a T cell costimulatory domain (e.g., from CD28, CD137, OX40, ICOS, or CD27) in tandem with a T cell triggering domain (e.g., from CD3). A T cell expressing a chimeric antigen receptor is referred to as a CAR T cell. Exemplary CAR T cells include CD19 targeted CTL019 cells (see, Grupp et al. (2015) BLOOD, 126:4983), 19-282 cells (see, Park et al. (2015) J. CLIN. ONCOL., 33:7010), and KTE-C19 cells (see, Locke et al. (2015) BLOOD, 126:3991). Additional exemplary CAR T cells are described in U.S. Pat. Nos. 7,446,190, 8,399,645, 8,906,682, 9,181,527, 9,272,002, 9,266,960, 10,253,086, 10640569, and 10,808,035, and International (PCT) Publication Nos. WO 2013/142034, WO 2015/120180, WO 2015/188141, WO 2016/120220, and WO 2017/040945. Exemplary approaches to express CARs using CRISPR systems are described in Hale et al. (2017) MOL THER METHODS CLIN DEV., 4:192, MacLeod et al. (2017) MOL THER, 25:949, and Eyquem et al. (2017) NATURE, 543:113.

In certain embodiments, an immune cell, e.g., a T cell, binds an antigen, e.g., a cancer antigen, through an endogenous T cell receptor (TCR). In certain embodiments, an immune cell, e.g., a T cell, is engineered to express an exogenous TCR, e.g., an exogenous naturally occurring TCR or an exogenous engineered TCR. T cell receptors comprise two chains referred to as the α- and β-chains, that combine on the surface of a T cell to form a heterodimeric receptor that can recognize MHC-restricted antigens. Each of α- and β-chain comprises a constant region and a variable region. Each variable region of the α- and β-chains defines three loops, referred to as complementary determining regions (CDRs) known as CDR1, CDR2, and CDR3 that confer the T cell receptor with antigen binding activity and binding specificity.

In certain embodiments, a CAR or TCR binds a cancer antigen selected from B-cell maturation antigen (BCMA), mesothelin, prostate specific membrane antigen (PSMA), prostate stem cell antigen (PSCA), carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD70, CD74, CD123, CD133, CD138, epithelial glycoprotein2 (EGP 2), epithelial glycoprotein-40 (EGP-40), epithelial cell adhesion molecule (EpCAM), receptor-type tyrosine-protein kinase (FLT3), folate-binding protein (FBP), fetal acetylcholine receptor (AChR), folate receptor-α and β (FRα and β), Ganglioside G2 (GD2), Ganglioside G3 (GD3), epidermal growth factor receptor 2 (HER-2/ERB2), epidermal growth factor receptor vIII (EGFRvIII), ERB3, ERB4, human telomerase reverse transcriptase (hTERT), Interleukin-13 receptor subunit alpha-2 (IL-13Ra2), K-light chain, kinase insert domain receptor (KDR), Lewis A (CA19.9), Lewis Y (LeY), LI cell adhesion molecule (LICAM), melanoma-associated antigen 1 (melanoma antigen family Al, MAGE-A1), Mucin 16 (MUC-16), Mucin 1 (MUC-1; e.g., a truncated MUC-1), KG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gpl00/HLA-A2, Glypican 3 (GPC3), HA-IH, HERK-V, IL-1 IRa, Latent Membrane Protein 1 (LMP1), Neural cell-adhesion molecule (N-CAM/CD56), and Trail Receptor (TRAIL-R).

Genetic loci suitable for insertion of a CAR- or exogenous TCR-encoding sequence include but are not limited to safe harbor loci (e.g., the AAVS1 locus) TCR subunit loci (e.g., the TCRα constant (TRAC) locus, the TCRβ constant 1 (TRBC1) locus, the TCRβ constant 2 (TRBC2) locus, the CD3E locus, the CD3D locus, the CD3G locus, and the CD3Z locus). It is understood that insertion in the TRAC locus reduces tonic CAR signaling and enhances T cell potency (see, Eyquem et al. (2017) NATURE, 543:113). Furthermore, inactivation of the endogenous TCR subunit gene, e.g., TRAC, TRBC1, or TRBC2 gene may reduce a graft-versus-host disease (GVHD) response, thereby allowing use of allogeneic T cells as starting materials for preparation of CAR T cells. Accordingly, in certain embodiments, an immune cell, e.g., a T cell, is engineered to have reduced expression of an endogenous TCR or TCR subunit, e.g., TRAC, TRBC1, TRBC2, CD3E, CD3D, CD3G, and/or CD3Z. The cell may be engineered to have partially reduced or no expression of the endogenous TCR or TCR subunit. For example, in certain embodiments, the immune cell, e.g., a T cell, is engineered to have less than 80% (e.g., less than 70%, less than 60%, less than 50%, less than 40%, less than 30%, less than 20%, less than 10%, or less than 5%) of the expression of the endogenous TCR or TCR subunit relative to a corresponding unmodified or parental cell. In certain embodiments, the immune cell, e.g., a T cell, is engineered to have no detectable expression of the endogenous TCR or TCR subunit. Exemplary approaches to reduce expression of TCRs using CRISPR systems are described in U.S. Pat. No. 9,181,527, Liu et al. (2017) CELL RES, 27:154, Ren et al. (2017) CLIN CANCER RES, 23:2255, Cooper et al. (2018) LEUKEMIA, 32:1970, and Ren et al. (2017) ONCOTARGET, 8:17002.

It is understood that certain immune cells, such as T cells, also express major histocompatibility complex (MHC) or human leukocyte antigen (HLA) genes, and inactivation of these endogenous gene may reduce an immune response, thereby allowing use of allogeneic T cells as starting materials for preparation of CAR T cells. Accordingly, in certain embodiments, an immune cell, e.g., a T-cell, is engineered to have reduced expression of one or more endogenous class I or class II MHCs or HLAs (e.g., beta 2-microglobulin (B2M), class II major histocompatibility complex transactivator (CIITA)). The cell may be engineered to have partially reduced or no expression of an endogenous MHC or HLA. For example, in certain embodiments, the immune cell, e.g., a T-cell, is engineered to have less than less than 80% (e.g., less than 70%, less than 60%, less than 50%, less than 40%, less than 30%, less than 20%, less than 10%, or less than 5%) of the expression of endogenous MHC (e.g., B2M, CIITA) relative to a corresponding unmodified or parental cell. In certain embodiments, the immune cell, e.g., a T cell, is engineered to have no detectable expression of an endogenous MHC (e.g., B2M, CIITA). In certain cases, a cell may be engineered to have expression of, e.g., HLA-E and/or HLA-G, in order to avoid attack by natural killer (NK) cells. Exemplary approaches to reduce expression of MHCs using CRISPR systems are described in Liu et al. (2017) CELL RES, 27:154, Ren et al. (2017) CLIN CANCER RES, 23:2255, and Ren et al. (2017) ONCOTARGET, 8:17002.

Other genes that may be inactivated include but are not limited to CD3, CD52, and deoxycytidine kinase (DCK). For example, inactivation of DCK may render the immune cells (e.g., T cells) resistant to purine nucleotide analogue (PNA) compounds, which are often used to compromise the host immune system in order to reduce a GVHD response during an immune cell therapy. In certain embodiments, the immune cell, e.g., a T-cell, is engineered to have less than less than 80% (e.g., less than 70%, less than 60%, less than 50%, less than 40%, less than 30%, less than 20%, less than 10%, or less than 5%) of the expression of endogenous CD52 or DCK relative to a corresponding unmodified or parental cell.

It is understood that the activity of an immune cell (e.g., T cell) may be enhanced by inactivating or reducing the expression of an immune suppressor such as an immune checkpoint protein. Accordingly, in certain embodiments, an immune cell, e.g., a T cell, is engineered to have reduced expression of an immune checkpoint protein. Exemplary immune checkpoint proteins expressed by wild-type T cells include but are not limited to PDCD1 (PD-1), CTLA4, ADORA2A (A2AR), B7-H3, B7-H4, BTLA, KIR, LAG3, HAVCR2 (TIM3), TIGIT, VISTA, PTPN6 (SHP-1), and FAS. The cell may be modified to have partially reduced or no expression of the immune checkpoint protein. For example, in certain embodiments, the immune cell, e.g., a T cell, is engineered to have less than 80% (e.g., less than 70%, less than 60%, less than 50%, less than 40%, less than 30%, less than 20%, less than 10%, or less than 5%) of the expression of the immune checkpoint protein relative to a corresponding unmodified or parental cell. In certain embodiments, the immune cell, e.g., a T cell, is engineered to have no detectable expression of the immune checkpoint protein. Exemplary approaches to reduce expression of immune checkpoint proteins using CRISPR systems are described in International (PCT) Publication No. WO 2017/017184, Cooper et al. (2018) LEUKEMIA, 32:1970, Su et al. (2016) ONCOIMMUNOLOGY, 6: c1249558, and Zhang et al. (2017) FRONT MED, 11:554.

The immune cell can be engineered to have reduced expression of an endogenous gene, e.g., an endogenous genes described above, by gene editing or modification. For example, in certain embodiments, an engineered CRISPR system disclosed herein may result in DNA cleavage at a gene locus, thereby inactivating the targeted gene. In other embodiments, an engineered CRISPR system disclosed herein may be fused to an effector domain (e.g., a transcriptional repressor or histone methylase) to reduce the expression of the target gene.

The immune cell can also be engineered to express an exogenous protein (besides an antigen-binding protein described above) at the locus of a human ADORA2A, B2M, CD52, CIITA, CTLA4, DCK, FAS, HAVCR2, LAG3, PDCD1, PTPN6, TIGIT, TRAC, TRBC1, TRBC2, CARD11, CD247, IL7R, LCK, or PLCG1 gene.

In certain embodiments, an immune cell, e.g., a T cell, is modified to express a dominant-negative form of an immune checkpoint protein. In certain embodiments, the dominant-negative form of the checkpoint inhibitor can act as a decoy receptor to bind or otherwise sequester the natural ligand that would otherwise bind and activate the wild-type immune checkpoint protein. Examples of engineered immune cells, for example, T cells containing dominant-negative forms of an immune suppressor are described, for example, in International (PCT) Publication No. WO 2017/040945.

In certain embodiments, an immune cell, e.g., a T cell, is modified to express a gene (e.g., a transcription factor, a cytokine, or an enzyme) that regulates the survival, proliferation, activity, or differentiation (e.g., into a memory cell) of the immune cell. In certain embodiments, the immune cell is modified to express TET2, FOXO1, IL-12, IL-15, IL-18, IL-21, IL-7, GLUT1, GLUT3, HK1, HK2, GAPDH, LDHA, PDK1, PKM2, PFKFB3, PGK1, ENO1, GYS1, and/or ALDOA. In certain embodiments, the modification is an insertion of a nucleotide sequence encoding the protein operably linked to a regulatory element. In certain embodiments, the modification is a substitution of a single nucleotide polymorphism (SNP) site in the endogenous gene. In certain embodiments, an immune cell, e.g., a T cell, is modified to express a variant of a gene, for example, a variant that has greater activity than the respective wild-type gene. In certain embodiments, the immune cell is modified to express a variant of CARD11, CD247, IL7R, LCK, or PLCG1. For example, certain gain-of-function variants of IL7R were disclosed in Zenatti et al., (2011) NAT. GENET. 43 (10): 932-39. The variant can be expressed from the native locus of the respective wild-type gene by delivering an engineered system described herein for targeting the native locus in combination with a donor template that carries the variant or a portion thereof.

In certain embodiments, an immune cell, e.g., a T cell, is modified to express a protein (e.g., a cytokine or an enzyme) that regulates the microenvironment that the immune cell is designed to migrate to (e.g., a tumor microenvironment). In certain embodiments, the immune cell is modified to express CA9, CA12, a V-ATPase subunit, NHE1, and/or MCT-1.

### A. Gene Therapies

It is understood that the engineered, non-naturally occurring system and CRISPR expression system, e.g., as disclosed herein, can be used to treat a genetic disease or disorder, i.e., a disease or disorder associated with or otherwise mediated by an undesirable mutation in the genome of a subject.

Exemplary genetic diseases or disorders include age-related macular degeneration, adrenoleukodystrophy (ALD), Alagille syndrome, alpha-1-antitrypsin deficiency, argininemia, argininosuccinic aciduria, ataxia (e.g., Friedreich ataxia, spinocerebellar ataxias, ataxia telangiectasia, essential tremor, spastic paraplegia), autism, biliary atresia, biotinidase deficiency, carbamoyl phosphate synthetase I deficiency, carbohydrate deficient glycoprotein syndrome (CDGS), a central nervous system (CNS)-related disorder (e.g., Alzheimer's disease, amyotrophic lateral sclerosis (ALS), canavan disease (CD), ischemia, multiple sclerosis (MS), neuropathic pain, Parkinson's disease), Bloom's syndrome, cancer, Charcot-Marie-Tooth disease (e.g., peroncal muscular atrophy, hereditary motor sensory neuropathy), congenital hepatic porphyria, citrullinemia, Crigler-Najjar syndrome, cystic fibrosis (CF), Dentatorubro-Pallidoluysian Atrophy (DRPLA), diabetes insipidus, Fabry, familial hypercholesterolemia (LDL receptor defect), Fanconi's anemia, fragile X syndrome, a fatty acid oxidation disorder, galactosemia, glucose-6-phosphate dehydrogenase (G6PD), glycogen storage diseases (e.g., type I (glucose-6-phosphatase deficiency, Von Gierke II (alpha glucosidase deficiency, Pompe), III (debrancher enzyme deficiency, Cori), IV (brancher enzyme deficiency, Anderson), V (muscle glycogen phosphorylase deficiency, McArdle), VII (muscle phosphofructokinase deficiency, Tauri), VI (liver phosphorylase deficiency, Hers), IX (liver glycogen phosphorylase kinase deficiency)), hemophilia A (associated with defective factor VIII), hemophilia B (associated with defective factor IX), Huntington's disease, glutaric aciduria, hypophosphatemia, Krabbe, lactic acidosis, Lafora disease, Leber's Congenital Amaurosis, Lesch Nyhan syndrome, a lysosomal storage disease, metachromatic leukodystrophy disease (MLD), mucopolysaccharidosis (MPS) (e.g., Hunter syndrome, Hurler syndrome, Maroteaux-Lamy syndrome, Sanfilippo syndrome, Scheie syndrome, Morquio syndrome, other, MPSI, MPSII, MPSIII, MSIV, MPS 7), a muscular/skeletal disorder (e.g., muscular dystrophy, Duchenne muscular dystrophy), myotonic Dystrophy (DM), neoplasia, N-acetylglutamate synthase deficiency, ornithine transcarbamylase deficiency, phenylketonuria, primary open angle glaucoma, retinitis pigmentosa, schizophrenia, Severe Combined Immune Deficiency (SCID), Spinobulbar Muscular Atrophy (SBMA), sickle cell anemia, Usher syndrome, Tay-Sachs disease, thalassemia (e.g., B-Thalassemia), trinucleotide repeat disorders, tyrosinemia, Wilson's disease, Wiskott-Aldrich syndrome, X-linked chronic granulomatous disease (CGD), X-linked severe combined immune deficiency, and xeroderma pigmentosum.

Additional exemplary genetic diseases or disorders and associated information are available on the world wide web at kumc.edu/gec/support, genome.gov/10001200, and ncbi.nlm.nih.gov/books/NBK22183/. Additional exemplary genetic diseases or disorders, associated genetic mutations, and gene therapy approaches to treat genetic diseases or disorders are described in International (PCT) Publication Nos. WO 2013/126794, WO 2013/163628, WO 2015/048577, WO 2015/070083, WO 2015/089354, WO 2015/134812, WO 2015/138510, WO 2015/148670, WO 2015/148860, WO 2015/148863, WO 2015/153780, WO 2015/153789, and WO 2015/153791, U.S. Pat. Nos. 8,383,604, 8,859,597, 8,956,828, 9,255, 130, and 9,273,296, and U.S. Patent Application Publication Nos. 2009/0222937, 2009/0271881, 2010/0229252, 2010/0311124, 2011/0016540, 2011/0023139, 2011/0023144, 2011/0023145, 2011/0023146, 2011/0023153, 2011/0091441, 2012/0159653, and 2013/0145487.

### VI. KITS

It is understood that the guide nucleic acid, the engineered, non-naturally occurring system, the CRISPR expression system, and/or a library disclosed herein can be packaged in a kit suitable for use by a medical provider. Accordingly, in another aspect, the invention provides kits containing any one or more of the elements disclosed in the above systems, libraries, methods, and compositions. In certain embodiments, the kit comprises an engineered, non-naturally occurring system as disclosed herein and instructions for using the kit. The instructions may be specific to the applications and methods described herein. In certain embodiments, one or more of the elements of the system are provided in a solution. In certain embodiments, one or more of the elements of the system are provided in lyophilized form, and the kit further comprises a diluent. Elements may be provided individually or in combinations, and may be provided in any suitable container, such as a vial, a bottle, a tube, or immobilized on the surface of a solid base (e.g., chip or microarray). In certain embodiments, the kit comprises one or more of the nucleic acids and/or proteins described herein. In certain embodiments, the kit provides all elements of the systems of the invention.

In certain embodiments of a kit comprising the engineered, non-naturally occurring dual guide system, the targeter nucleic acid and the modulator nucleic acid are provided in separate containers. In other embodiments, the targeter nucleic acid and the modulator nucleic acid are pre-complexed, and the complex is provided in a single container.

In certain embodiments, the kit comprises a Cas protein or a nucleic acid comprising a regulatory element operably linked to a nucleic acid encoding a Cas protein provided in a separate container. In other embodiments, the kit comprises a Cas protein pre-complexed with the single guide nucleic acid or a combination of the targeter nucleic acid and the modulator nucleic acid, and the complex is provided in a single container.

In certain embodiments, the kit further comprises one or more donor templates provided in one or more separate containers. In certain embodiments, the kit comprises a plurality of donor templates as disclosed herein (e.g., in separate tubes or immobilized on the surface of a solid base such as a chip or a microarray), one or more guide nucleic acids disclosed herein, and optionally a Cas protein or a regulatory element operably linked to a nucleic acid encoding a Cas protein as disclosed herein. Such kits are useful for identifying a donor template that introduces optimal genetic modification in a multiplex assay. The CRISPR expression systems as disclosed herein are also suitable for use in a kit.

In certain embodiments, a kit further comprises one or more reagents and/or buffers for use in a process utilizing one or more of the elements described herein. Reagents may be provided in any suitable container and may be provided in a form that is usable in a particular assay, or in a form that requires addition of one or more other components before use (e.g., in concentrate or lyophilized form). A buffer may be a reaction or storage buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In certain embodiments, the buffer has a pH from about 7 to about 10. In certain embodiments, the kit further comprises a pharmaceutically acceptable carrier. In certain embodiments, the kit further comprises one or more devices or other materials for administration to a subject.

### VII. EMBODIMENTS

In embodiment 1 provided herein is a composition comprising a modified human cell comprising (a) a first genomic modification comprising a first portion of a first polynucleotide, wherein the first portion codes for a first chimeric antigen receptor (CAR) or portion thereof, inserted into a site with a TRAC gene, whereby the TRAC gene is partially or completely inactivated and the first CAR or portion thereof is expressed, and (b) a second genomic modification comprising a second polynucleotide coding for a fusion protein of B2M and HLA-E or HLA-G inserted into a B2M gene, whereby endogenous B2M is partially or completely inactivated and the fusion protein is expressed. In embodiment 2 provided herein is the composition of embodiment 1, wherein the TRAC gene is completely inactivated. In embodiment 3 provided herein is the composition of embodiment 1 or embodiment 2, wherein the endogenous B2M gene is completely inactivated. In embodiment 4 provided herein is the composition of any one of embodiments 1-3, further comprising (c) a third genomic modification in a CIITA gene, wherein the CIITA gene is partially or completely inactivated. In embodiment 5 provided herein is the composition of embodiment 4, wherein the CIITA gene is completely inactivated. In embodiment 6 provided herein is the composition of embodiment 4 or embodiment 5, wherein the third genomic modification comprises a substitution, an insertion, a deletion, a nonsense mutation, or a truncation. In embodiment 7 provided herein is the composition of any one of embodiments 1 through 6, wherein the CAR or portion thereof comprises a polypeptide that binds at least one of B7H3, BCMA, GPRC5D, CD8, CD8a, CD19, CD20, CD22, CD28, 4-1BB, or CD3zeta. In embodiment 8 provided herein is the composition of embodiment 7, wherein the CAR or portion thereof comprises a polypeptide at least 60, at least 70, at least 80, at least 90, at least 95, at least 99%, or 100% identical to any one of the amino acid sequences of SEQ ID NOs: 86-124. In embodiment 9 provided herein is the composition of embodiment 1 or embodiment 6, wherein the CAR or portion thereof comprises a polypeptide that binds at least one of B7H3, BCMxA, GPRC5D, CD8, CD8a, CD20, CD22, CD28, 4-1BB, or CD3zeta. In embodiment 10 provided herein is the composition of embodiment 9, wherein the CAR or portion thereof comprises a polypeptide at least 60, at least 70, at least 80, at least 90, at least 95, at least 99%, or 100% identical to any one of the amino acid sequences of SEQ ID NOS: 86-104 or 116-124. In embodiment 11 provided herein is the composition of any one of embodiments 1 through 10, further comprising a second portion of the first polynucleotide, wherein the second portion codes for a second CAR or portion thereof, different from the first CAR or portion thereof. In embodiment 12 provided herein is a composition comprising a modified human cell comprising (a) a first genomic modification comprising a first portion of a polynucleotide, wherein the first portion codes for a first chimeric antigen receptor (CAR) or portion thereof, inserted into a site with a TRAC gene, whereby the TRAC gene is partially or completely inactivated and the first CAR or portion thereof is expressed, and (b) a second genomic modification in a CIITA gene, wherein the CIITA gene is partially or completely inactivated. In embodiment 13 provided herein is the composition of embodiment 12, wherein the TRAC gene is completely inactivated. In embodiment 14 provided herein is the composition of embodiment 12 or embodiment 13, wherein the CIITA gene is completely inactivated. In embodiment 15 provided herein is the composition of any one of embodiments 12 through 14, further comprising (c) a third genomic modification comprising a polynucleotide coding for a fusion protein of B2M and HLA-E or HLA-G inserted into a B2M gene, whereby endogenous B2M is partially or completely inactivated and the fusion protein is expressed. In embodiment 16 provided herein is the composition of embodiment 15, wherein endogenous B2M is completely inactivated. In embodiment 17 provided herein is the composition of embodiment 12, wherein the second genomic modification comprises a substitution, an insertion, a deletion, a nonsense mutation, or a truncation. In embodiment 18 provided herein is the composition of any one of embodiments 12 through 17, wherein the CAR or portion thereof comprises a polypeptide that binds at least one of B7H3, BCMA, GPRC5D, CD8, CD8a, CD19, CD20, CD22, CD28, 4-1BB, or CD3zeta. In embodiment 19 provided herein is the composition of embodiment 18, wherein the CAR or portion thereof comprises a polypeptide at least 60, at least 70, at least 80, at least 90, at least 95, at least 99%, or 100% identical to any one of the amino acid sequences of SEQ ID NOs: 86-124. In embodiment 20 provided herein is the composition of any one of embodiments 12 through 17, wherein the CAR or portion thereof comprises a polypeptide that binds at least one of B7H3, BCMA, GPRC5D, CD8, CD8a, CD20, CD22, CD28, 4-1BB, or CD3zeta. In embodiment 21 provided herein is the composition of embodiment 20, wherein the CAR or portion thereof comprises a polypeptide at least 60, at least 70, at least 80, at least 90, at least 95, at least 99%, or 100% identical to any one of the amino acid sequences of SEQ ID NOs: 86-104 or 116-124. In embodiment 22 provided herein is the composition of any one of embodiments 12 through 21, further comprising a second portion of the polynucleotide, wherein the second potion codes for a second CAR or portion thereof, different from the first CAR or portion thereof. In embodiment 23 provided herein is a composition comprising a modified human cell comprising (a) a first genomic modification comprising a polynucleotide coding for a fusion protein of B2M and HLA-E or HLA-G inserted into a B2M gene, whereby endogenous B2M is partially or completely inactivated and the fusion protein is expressed; and (b) a second genomic modification in a CIITA gene, wherein the CIITA gene is partially or completely inactivated. In embodiment 24 provided herein is the composition of embodiment 23, wherein the endogenous B2M gene is completely inactivated. In embodiment 25 provided herein is the composition of embodiment 23 or embodiment 24, wherein the CIITA gene is completely inactivated. In embodiment 26 provided herein is the composition of embodiment 25, wherein the second genomic modification comprises a substitution, an insertion, a deletion, a nonsense mutation, or a truncation. In embodiment 27 provided herein is the composition of any one of embodiments 23 through 26, further comprising (c) a third genomic modification comprising a first portion of a polynucleotide, wherein the first portion codes for a first chimeric antigen receptor (CAR) or portion thereof, inserted into a site with a TRAC gene, whereby the TRAC gene is partially or completely inactivated and the first CAR or portion thereof is expressed. In embodiment 28 provided herein is the composition of embodiment 27, wherein the CAR or portion thereof comprises a polypeptide that binds at least one of B7H3, BCMA, GPRC5D, CD8, CD8a, CD19, CD20, CD22, CD28, 4-1BB, or CD3zeta. In embodiment 29 provided herein is the composition of embodiment 28, wherein the CAR or portion thereof comprises a polypeptide at least 60, at least 70, at least 80, at least 90, at least 95, at least 99%, or 100% identical to any one of the amino acid sequences of SEQ ID NOs: 86-124. In embodiment 30 provided herein is the composition of embodiment 27, wherein the CAR or portion thereof comprises a polypeptide that binds at least one of B7H3, BCMxA, GPRC5D, CD8, CD8a, CD20, CD22, CD28, 4-1BB, or CD3zeta. In embodiment 31 provided herein is the composition of embodiment 29, wherein the CAR or portion thereof comprises a polypeptide at least 60, at least 70, at least 80, at least 90, at least 95, at least 99%, or 100% identical to any one of the amino acid sequences of SEQ ID NOS: 86-104 or 116-124. In embodiment 32 provided herein is the composition of any one of embodiments 27 through 31, further comprising a second portion of the first polynucleotide, wherein the second portion codes for a second CAR or portion thereof, different from the first CAR or portion thereof. In embodiment 33 provided herein is the composition of any one of embodiments 1 through 32, wherein the cell comprises an immune cell or a stem cell. In embodiment 34 provided herein is the composition of embodiment 33, wherein the cell comprises an immune cell comprising a neutrophil, cosinophil, basophil, mast cell, monocyte, macrophage, dendritic cell, natural killer cell, or a lymphocyte. In embodiment 35 provided herein is the composition of embodiment 33, wherein the cell comprises a T cell. In embodiment 36 provided herein is the composition of embodiment 33, wherein the cell comprises a stem cell comprising a human pluripotent, multipotent stem cell, embryonic stem cell, induced pluripotent stem cell, hematopoictic stem cell, or a CD34+ cell. In embodiment 37 provided herein is the composition of embodiment 33, wherein the cell comprises a stem cell comprising an iPSC. In embodiment 38 provided herein is the composition of any one of embodiments 1 through 37, further comprising a nuclease system or one or more polynucleotides encoding for one or more parts of the system comprising (1) a nucleic acid-guided nuclease; and (2) a guide nucleic acid compatible with and capable of binding to and activating the nucleic acid-guided nuclease and comprising a spacer sequence complementary to a target nucleotide sequence in a polynucleotide of a human genome, wherein, contacting the target polynucleotide with the nuclease system results in a strand break in at least one strand of the target polynucleotide of the genome of the human cell at or near the target nucleotide sequence. In embodiment 39 provided herein is the composition of embodiment 38, wherein the nucleic acid-guided nuclease comprises an engineered, non-naturally occurring nuclease. In embodiment 40 provided herein is the composition of embodiment 38 or embodiment 39, wherein the nucleic acid-guided nuclease comprises a Class 1 or a Class 2 nuclease. In embodiment 41 provided herein is the composition of embodiment 40, wherein the nucleic acid-guided nuclease comprises a Type II or a Type V nuclease. In embodiment 42 provided herein is the composition of embodiment 41, wherein the nucleic acid-guided nuclease comprises a Type V-A, V-B, V-C, V-D, or V-E nuclease. In embodiment 43 provided herein is the composition of embodiment 42, wherein the nucleic acid-guided nuclease comprises a Type V-A nuclease. In embodiment 44 provided herein is the composition of embodiment 43, wherein the nucleic acid-guided nuclease comprises a MAD nuclease, an ART nuclease, or an ABW nuclease. In embodiment 45 provided herein is the composition of embodiment 44, wherein the nucleic acid-guided nuclease comprises an amino acid sequence at least 80%, at least 85%, at least 90%, at least 95%, at least 99%, or 100% identical to an amino acid sequence of a MAD, ART, or ABW nuclease. In embodiment 46 provided herein is the composition of embodiment 44, wherein the nucleic acid-guided nuclease comprises a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20 nuclease. In embodiment 47 provided herein is the composition of embodiment 44, wherein the nucleic acid-guided nuclease comprises an ART1, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35 nuclease. In embodiment 48 provided herein is the composition of embodiment 44, wherein the nucleic acid-guided nuclease comprises an amino acid sequence at least 80%, at least 85%, at least 90%, at least 95%, at least 99%, or 100% identical, to the amino acid sequence of MAD2, MAD7, ART2, ART11, or ART11*. In embodiment 49 provided herein is the composition of embodiment 44, wherein the nucleic acid-guided nuclease comprises an amino acid sequence that is at least 80%, at least 85%, at least 90%, at least 95%, 99%, or 100% identical, to the amino acid sequence of SEQ ID NO: 37. In embodiment 50 provided herein is the composition of any one of embodiments 38 through 49, wherein the nucleic acid-guided nuclease further comprises at least one nuclear localization signal (NLS), at least one purification tag, and/or at least one cleavage site. In embodiment 51 provided herein is the composition of embodiment 50, wherein the nucleic acid-guided nuclease comprises at least 4 nuclear localization signals (NLS). In embodiment 52 provided herein is the composition of embodiment 51, wherein the nucleic acid-guided nuclease comprises one N-terminal and three C-terminal nuclease localization signals (NLS). In embodiment 53 provided herein is the composition of any one of embodiments 50 through 52, wherein the nuclear localization signals comprise any one of SEQ ID NOs: 40-56. In embodiment 54 provided herein is the composition of embodiment 32, wherein the NLS comprises SEQ ID NOs: 40, 51, and 56. In embodiment 55 provided herein is the composition of embodiment 38, wherein the guide nucleic acid comprises (i) a targeter nucleic acid comprising a targeter stem sequence and the spacer sequence, and (ii) a modulator nucleic acid comprising a modulator stem sequence complementary to the targeter stem sequence, and, optionally, a 5′ sequence. In embodiment 56 provided herein is the composition of embodiment 55, wherein the guide nucleic acid comprises a single polynucleotide. In embodiment 57 provided herein is the composition of embodiment 55 or embodiment 56, wherein the guide nucleic acid comprises an engineered, non-naturally occurring guide nucleic acid. In embodiment 58 provided herein is the composition of embodiment 55 or embodiment 57, wherein the guide nucleic acid comprises a dual guide nucleic acid, wherein the targeter nucleic acid and the modulator nucleic acid are separate polynucleotides. In embodiment 59 provided herein is the composition of embodiment 58, wherein the dual guide nucleic acid is capable of binding to and activating a nucleic acid-guided nuclease, that, in a naturally occurring system, is activated by a single crRNA in the absence of a tracrRNA. In embodiment 60 provided herein is the composition of any one of embodiments 38 through 59, wherein the target nucleotide sequence is within at least 10, 20, 30, 40, or 50 nucleotides of a protospacer adjacent motif (PAM) that is recognized by the nucleic acid-guided nuclease. In embodiment 61 provided herein is the composition of any one of embodiments 38 through 60, wherein the guide nucleic acid and the nucleic acid-guided nuclease form a nucleic acid-guided nuclease complex. In embodiment 62 provided herein is the composition of embodiment 61, wherein the guide nucleic acid further comprises a donor template recruiting sequence. In embodiment 63 provided herein is the composition of embodiment 38 through 62, wherein the guide nucleic acid comprises a heterologous spacer sequence. In embodiment 64 provided herein is the composition of any one of embodiments 38 through 63, wherein the spacer sequence is at least 80%, at least 85%, at least 90%, at least 95%, at least 99.5%, or 100% identical to any one of any one of SEQ ID NOs: 125-2019. In embodiment 65 provided herein is the composition of any one of embodiments 38 through 64, wherein some or all of the guide nucleic acid comprises RNA. In embodiment 66 provided herein is the composition of embodiment 65, wherein at least 50%, at least 70%, at least 90%, at least 95%, or 100% of the guide nucleic acid comprises RNA. In embodiment 67 provided herein is the composition of any one of embodiments 38 through 66, wherein the guide nucleic acid comprises one or more chemical modifications to one or more nucleotides and/or internucleotide linkages at or near the 5′ end, at or near the 3′ end, and/or both. In embodiment 68 provided herein is the composition of embodiment 67, wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, or a combination thereof. In embodiment 69 provided herein is the composition of any one of embodiments 38 through 68, further comprising one or more donor templates. In embodiment 70 provided herein is the composition of embodiment 69, wherein the donor template comprises single-stranded DNA, linear single-stranded RNA, linear double-stranded DNA, linear double-stranded RNA, circular single-stranded DNA, circular single-stranded RNA, circular double-stranded DNA, or circular double-stranded RNA. In embodiment 71 provided herein is the composition of embodiment 69 or embodiment 70, wherein the donor template comprises two homology arms. In embodiment 72 provided herein is the composition of embodiment 71, wherein the homology arms comprise at least 50, 100, 150, 200, 250, 300, 350, 400, 450, 500, 600, 700, 800, or 900 and/or at most 100, 150, 200, 250, 300, 350, 400, 450, 500, 600, 700, 800, 900, or 1000 nucleotides, for example 50-1000 nucleotides, preferably 100-800 nucleotides, more preferably 250-750 nucleotides, even more preferably 400-600 nucleotides. In embodiment 73 provided herein is the composition of any one of embodiments embodiment 69 through 72, wherein the donor template comprises a mutation in a PAM sequence to partially or completely abolish binding of the RNP to the DNA. In embodiment 74 provided herein is the composition of any one of embodiments 69 through 73, wherein the donor template comprises one or more promoters. In embodiment 75 provided herein is the composition of embodiment 74, wherein the promoter shares at least 70%, at least 75%, at least 80%, at least 85%, at least 90%, at least 95%, at least 99.5%, or 100% sequence identity with any one of SEQ ID NOs: 78-85. In embodiment 76 provided herein is the composition of any one of embodiments 69 through 75, wherein the donor template comprises one or more chemical modifications to one or more nucleotides and/or internucleotide linkages at or near the 5′ end, at or near the 3′ end, or both. In embodiment 77 provided herein is the composition of embodiment 76, wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, a suitable alternative, or a combination thereof. In embodiment 78 provided herein is the composition of any one of embodiments 69 through 77, wherein the at least portion of the donor template is inserted by an innate cell repair mechanism. In embodiment 79 provided herein is the composition of embodiment 78, wherein the innate cell repair mechanism comprises homology directed repair (HDR). In embodiment 80 provided herein is a composition comprising a plurality of cell populations comprising (a) a first cell population comprising a plurality of the modified human cells of any one of embodiments 1 through 11, and (b) a second cell population comprising a plurality of modified human cells wherein the second cell population does not comprise a modified human cell of the first population. In embodiment 81 provided herein is the composition of embodiment 80, wherein the first population of cells comprises at least 1, 2, 3, 4, 5, 6, 7, 8, 9, 10, 15, 20, 25, 30, 35, 40, 45, 50, 55, 60, 65, or 70% and/or not more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations, preferably 1-10%, more preferably 1-20%, even more preferably 1-30%, yet even more preferably 1-40%. In embodiment 82 provided herein is the composition of embodiment 80 or embodiment 81, wherein the second population of cells comprises at least 1, 2, 3, 4, 5, 6, 7, 8, 9, 10, 15, 20, 25, 30, 35, 40, 45, 50, 55, 60, 65, or 70% and/or no more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations, preferably 1-10%, more preferably 1-20%, even more preferably 1-30%, yet even more preferably 1-40%. In embodiment 83 provided herein is the composition of any one of embodiments 80 through 82, further comprising a third cell population wherein the third cell population does not contain a modified human cell of either the first or the second cell population. In embodiment 84 provided herein is the composition of embodiment 83, wherein the third population of cells comprises at least 1, 2, 3, 4, 5, 6, 7, 8, 9, 10, 15, 20, 25, 30, 35, 40, 45, 50, 55, 60, 65, or 70% and/or no more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations, preferably 1-10%, more preferably 1-20%, even more preferably 1-30%, yet even more preferably 1-40%. In embodiment 85 provided herein is the composition of any one of embodiments 80 through 84, further comprising a fourth cell population wherein the fourth cell population does not contain a modified human cell of either the first, second, or third cell population. In embodiment 86 provided herein is the composition of embodiment 85, wherein the fourth population of cells comprises at least 1, 2, 3, 4, 5, 6, 7, 8, 9, 10, 15, 20, 25, 30, 35, 40, 45, 50, 55, 60, 65, or 70% and/or no more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations, preferably 1-10%, more preferably 1-20%, even more preferably 1-30%, yet even more preferably 1-40%. In embodiment 87 provided herein is a composition comprising a plurality of cell populations comprising (a) a first cell population comprising a plurality of the modified human cells of any one of embodiments 4 through 11, and (b) a second cell population comprising a plurality of modified human cells wherein the second cell population does not comprise a modified human cell of any one of embodiments 4 through 11. In embodiment 88 provided herein is the composition of embodiment 87 further comprising a third cell population wherein the third cell population does not contain a modified human cell of embodiment 4 through 11 or a modified human cell of the second cell population. In embodiment 89 provided herein is the composition of any one of embodiments 80 through 88, further comprising a pharmaceutically acceptable excipient.

In embodiment 90 provided herein is a composition comprising a plurality of cell populations comprising (a) a first cell population comprising a plurality of cells wherein each cell comprises (i) a first genomic modification whereby a first gene that codes for a subunit of a TCR is partially or completely inactivated, (ii) a second genomic modification whereby a second gene that codes for a subunit of an HLA-1 protein is partially or completely inactivated, (iii) a third genomic modification whereby a third gene that codes for a subunit of an HLA-2 protein or that codes for a transcription factor for one or more subunits of an HLA-2 protein is partially or completely inactivated, and (b) a second cell population, different from the first, wherein the second cell population comprises a plurality of cells that do not comprise one or more of genomic modifications of (i) through (iii), wherein each cell of the second population comprises the same genomic modifications. In embodiment 91 provided herein is the composition of embodiment 90, wherein the first cell population comprises at least 1, 2, 3, 4, 5, 6, 7, 8, 9, 10, 15, 20, 25, 30, 35, 40, 45, 50, 55, 60, 65, or 70% and/or no more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations, preferably 1-10%, more preferably 1-20%, even more preferably 1-30%, yet even more preferably 1-40%. In embodiment 92 provided herein is the composition of embodiment 90 or embodiment 91, wherein the second cell population comprises at least 1, 2, 3, 4, 5, 6, 7, 8, 9, 10, 15, 20, 25, 30, 35, 40, 45, 50, 55, 60, 65, or 70% and/or no more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations, preferably 1-10%, more preferably 1-20%, even more preferably 1-30%, yet even more preferably 1-40%. In embodiment 93 provided herein is the composition of any one of embodiments 90 through 92, wherein the first cell population further comprises (iv) a fourth genomic modification comprising a first portion of a polynucleotide, wherein the first portion codes for a first chimeric antigen receptor (CAR) or portion thereof, inserted into the first gene coding for a subunit of the T cell receptor (TCR) or into a safe harbor site, whereby the first CAR or portion thereof is expressed. In embodiment 94 provided herein is the composition of embodiment 93, wherein the subunit of a TCR protein comprises an alpha subunit or a beta subunit or a TRAC, TRBC, CD3E, CD3D, CD3G, or CD3Z protein. In embodiment 95 provided herein is the composition of embodiment 94, wherein the subunit of a TCR protein is an alpha subunit. In embodiment 96 provided herein is the composition of embodiment 95, wherein the gene coding for the subunit of a TCR protein is a TRAC gene. In embodiment 97 provided herein is the composition of embodiment 90 or embodiment 96, wherein the first cell population further comprises (v) a fifth genomic modification comprising a polynucleotide coding for a fusion protein of B2M and a subunit of an HLA-1 protein inserted into a site within the second gene or a safe harbor site, whereby the fusion protein is expressed. In embodiment 98 provided herein is the composition of embodiment 97, wherein the first subunit comprises B2M. In embodiment 99 provided herein is the composition of embodiment 97 or embodiment 98, wherein the subunit of an HLA-1 protein comprises HLA-C, HLA-E, or HLA-G. In embodiment 100 provided herein is the composition of embodiment 99, wherein the subunit of an HLA-1 protein comprises HLA-E or HLA-G. In embodiment 101 provided herein is the composition of embodiment 99, wherein the subunit of an HLA-1 protein comprises HLA-E. In embodiment 102 provided herein is the composition of embodiment 99, wherein the subunit of an HLA-1 protein comprises HLA-G. In embodiment 103 provided herein is the composition of any one of embodiments 90 through 102, further comprising a third cell population wherein the third cell population does not contain a modified human cell of either the first or the second cell population. In embodiment 104 provided herein is the composition of embodiment 103, wherein the third cell population comprises at least 1, 2, 3, 4, 5, 6, 7, 8, 9, 10, 15, 20, 25, 30, 35, 40, 45, 50, 55, 60, 65, or 70% and/or no more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations, preferably 1-10%, more preferably 1-20%, even more preferably 1-30%, yet even more preferably 1-40%. In embodiment 105 provided herein is the composition of any one of embodiments 90 through 104, further comprising a fourth cell population wherein the fourth cell population does not contain a modified human cell of either the first, second, or third cell population. In embodiment 106 provided herein is the composition of embodiment 105, wherein the cell population comprises at least 1, 2, 3, 4, 5, 6, 7, 8, 9, 10, 15, 20, 25, 30, 35, 40, 45, 50, 55, 60, 65, or 70% and/or no more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations, preferably 1-10%, more preferably 1-20%, even more preferably 1-30%, yet even more preferably 1-40%. In embodiment 107 provided herein is the composition of any one of embodiments 90 to 106, wherein the cell populations comprise immune cells or stem cells. In embodiment 108 provided herein is the composition of embodiment 107, wherein the cell populations comprise immune cells comprising neutrophils, eosinophils, basophils, mast cells, monocytes, macrophages, dendritic cells, natural killer cells, or a lymphocyte. In embodiment 109 provided herein is the composition of embodiment 107, wherein the cell populations comprise immune cells comprising T cells. In embodiment 110 provided herein is the composition of embodiment 107, wherein the cell populations comprise stem cells comprising human pluripotent stem cells, multipotent stem cells, embryonic stem cells, induced pluripotent stem cells (iPSC), hematopoietic stem cells, or a CD34+ cells. In embodiment 111 provided herein is the composition of embodiment 107, wherein the cell populations comprise stem cells comprising induced pluripotent stem cells (iPSC).

In embodiment 112 provided herein is a composition comprising a cell comprising a first nucleic acid-guided nuclease system comprising (a) a first nucleic acid-guided nuclease comprising a Type V CRISPR endonuclease, and (b) a first guide nucleic acid, compatible with the first nucleic acid-guided nuclease, comprising a spacer sequence directed at a first target nucleotide sequence in a gene coding for a first subunit of an HLA-1 protein, wherein the first nucleic acid-guided nuclease and the first guide nucleic acid, when complexed, target and cleave at least one strand of DNA at a site at or near the first target nucleotide sequence in the gene coding for the first subunit of an HLA-1 protein. In embodiment 113 provided herein is the composition of embodiment 112, wherein the first subunit comprises B2M. In embodiment 114 provided herein is the composition of embodiment 112, wherein the cell further comprises a first donor template comprising a polynucleotide coding for a fusion protein comprising B2M and a second subunit of an HLA-1 protein. In embodiment 115 provided herein is the composition of embodiment 114, wherein the second subunit of an HLA-1 protein comprises HLA-C, HLA-E, or HLA-G. In embodiment 116 provided herein is the composition of embodiment 114, wherein the second subunit of an HLA-1 protein comprises HLA-E or HLA-G. In embodiment 117 provided herein is the composition of embodiment 114, wherein the second subunit of an HLA-1 protein comprises HLA-E. In embodiment 118 provided herein is the composition of embodiment 114, wherein the second subunit of an HLA-1 protein comprises HLA-G. In embodiment 119 provided herein is the composition of any one of embodiments 112 to 118, wherein the cell further comprises a second nucleic acid-guided nuclease system comprising (c) a second nucleic acid-guided nuclease comprising a Type V CRISPR endonuclease, and (d) a second guide nucleic acid, compatible with the second nucleic acid-guided nuclease, comprising a spacer sequence directed at a second target nucleotide sequence in a gene coding for a subunit of an HLA-2 protein or a transcription factor regulating the expression of one or more subunits of an HLA-2 protein, wherein the second nucleic acid-guided nuclease and the second guide nucleic acid, when complexed, target and cleave at least one strand of DNA at a site at or near the second target nucleotide sequence in the gene coding for a subunit of an HLA-2 protein or a transcription factor regulating the expression of one or more subunits of an HLA-2 protein. In embodiment 120 provided herein is the composition of embodiment 119, wherein the transcription factor comprises CIITA. In embodiment 121 provided herein is the composition of any one of embodiments 112 to 120, wherein the cell further comprises a third nucleic acid-guided nuclease system comprising (e) a third nucleic acid-guided nuclease comprising a Type V CRISPR endonuclease, and (f) a third guide nucleic acid, compatible with the third nucleic acid-guided nuclease, comprising a spacer sequence directed at a third target nucleotide sequence in a gene coding for a subunit of a TCR protein, wherein the third nucleic acid-guided nuclease and the third guide nucleic acid, when complexed, target and cleave at least one strand of DNA at a site at or near the third target nucleotide sequence in the gene coding for the subunit of a TCR protein. In embodiment 122 provided herein is the composition of embodiment 121, wherein the subunit of a TCR protein comprises an alpha subunit or a beta subunit or a TRAC, TRBC, CD3E, CD3D, CD3G, or CD3Z protein. In embodiment 123 provided herein is the composition of embodiment 122, wherein the subunit of a TCR protein is an alpha subunit. In embodiment 124 provided herein is the composition of embodiment 121, wherein the gene coding for the subunit of a TCR protein is a TRAC gene. In embodiment 125 provided herein is the composition of any one of embodiments 121 through 124, wherein the cell further comprises a donor template comprising a polynucleotide coding for a first chimeric antigen receptor (CAR) or portion thereof. In embodiment 126 provided herein is the composition of embodiment 125, wherein the CAR or portion thereof comprises a polypeptide that binds at least one of B7H3, BCMA, GPRC5D, CD8, CD8a, CD19, CD20, CD22, CD28, 4-1BB, or CD3zeta. In embodiment 127 provided herein is the composition of embodiment 126, wherein the CAR or portion thereof comprises a polypeptide at least 60, at least 70, at least 80, at least 90, at least 95, at least 99%, or 100% identical to any one of the amino acid sequences of SEQ ID NOs: 86-124. In embodiment 128 provided herein is the composition of embodiment 125, wherein the CAR or portion thereof comprises a polypeptide that binds at least one of B7H3, BCMxA, GPRC5D, CD8, CD8a, CD20, CD22, CD28, 4-1BB, or CD3zeta. In embodiment 129 provided herein is the composition of embodiment 128, wherein the CAR or portion thereof comprises a polypeptide at least 60, at least 70, at least 80, at least 90, at least 95, at least 99%, or 100% identical to any one of the amino acid sequences of SEQ ID NOs: 86-104 or 116-124. In embodiment 130 provided herein is a composition comprising a cell comprising a first nucleic acid-guided nuclease system comprising (a) a first nucleic acid-guided nuclease comprising a Type V CRISPR endonuclease, and (b) a first guide nucleic acid, compatible with the first nucleic acid-guided nuclease, comprising a spacer sequence directed at a first target nucleotide sequence in a gene coding for a subunit of an HLA-2 protein, or to a transcription factor regulating expression of one or more genes coding for one or more subunits of HLA-2 proteins, wherein the first nucleic acid-guided nuclease and the first guide nucleic acid, when complexed, target and cleave at least one strand of DNA at a site at or near the first target nucleotide sequence in the gene coding for a subunit of an HLA-2 protein, or to a transcription factor regulating expression of one or more genes coding for one or more subunits of HLA-2 proteins. In embodiment 131 provided herein is the composition of embodiment 130, wherein the transcription factor comprises CIITA. In embodiment 132 provided herein is the composition of embodiment 130 or 131, wherein the cell further comprises a second nucleic acid-guided nuclease system comprising (c) a second nucleic acid-guided nuclease comprising a Type V CRISPR endonuclease, and (d) a second guide nucleic acid, compatible with the second nucleic acid-guided nuclease, comprising a spacer sequence directed at a second target nucleotide sequence in a gene coding for a subunit of a TCR protein, wherein the second nucleic acid-guided nuclease and the second guide nucleic acid, when complexed, target and cleave at least one strand of DNA at a site at or near the second target nucleotide sequence in the gene coding for the subunit of a TCR protein. In embodiment 133 provided herein is the composition of embodiment 132, wherein the subunit of a TCR protein comprises an alpha subunit or a beta subunit or a TRAC, TRBC, CD3E, CD3D, CD3G, or CD3Z protein. In embodiment 134 provided herein is the composition of embodiment 133, wherein the subunit of a TCR protein is an alpha subunit. In embodiment 135 provided herein is the composition of embodiment 132, wherein the gene coding for the subunit of a TCR protein is a TRAC gene. In embodiment 136 provided herein is the composition of any one of embodiments 132 through 135, wherein the cell further comprises a donor template comprising a polynucleotide coding for a first chimeric antigen receptor (CAR) or portion thereof. In embodiment 137 provided herein is the composition of embodiment 136, wherein the CAR or portion thereof comprises a polypeptide that binds at least one of B7H3, BCMA, GPRC5D, CD8, CD8a, CD19, CD20, CD22, CD28, 4-1BB, or CD3zeta. In embodiment 138 provided herein is the composition of embodiment 137, wherein the CAR or portion thereof comprises a polypeptide at least 60, at least 70, at least 80, at least 90, at least 95, at least 99%, or 100% identical to any one of the amino acid sequences of SEQ ID NOs: 86-124. In embodiment 139 provided herein is the composition of embodiment 136, wherein the CAR or portion thereof comprises a polypeptide that binds at least one of B7H3, BCMxA, GPRC5D, CD8, CD8a, CD20, CD22, CD28, 4-1BB, or CD3zeta. In embodiment 140 provided herein is the composition of embodiment 139, wherein the CAR or portion thereof comprises a polypeptide at least 60, at least 70, at least 80, at least 90, at least 95, at least 99%, or 100% identical to any one of the amino acid sequences of SEQ ID NOs: 86-104 or 116-124. In embodiment 141 provided herein is a composition comprising a cell comprising a first nucleic acid-guided nuclease system comprising (a) a first nucleic acid-guided nuclease comprising a Type V CRISPR endonuclease, and (b) a first guide nucleic acid, compatible with the nucleic acid-guided nuclease, comprising a spacer sequence directed at a first target nucleotide sequence in a gene coding for a subunit of a TCR protein, wherein the first nucleic acid-guided nuclease and the first guide nucleic acid, when complexed, target and cleave at least one strand of DNA at a site at or near the first target nucleotide sequence in the gene coding for the subunit of a TCR protein. In embodiment 142 provided herein is the composition of embodiment 141, wherein the subunit of a TCR protein comprises an alpha subunit or a beta subunit or a TRAC, TRBC, CD3E, CD3D, CD3G, or CD3Z protein. In embodiment 143 provided herein is the composition of embodiment 142, wherein the subunit of a TCR protein is an alpha subunit. In embodiment 144 provided herein is the composition of any one of embodiment 141, wherein the gene coding for the subunit of a TCR protein is a TRAC gene. In embodiment 145 provided herein is the composition of any one of embodiments 141 through 144, wherein the cell further comprises a donor template comprising a polynucleotide coding for a first chimeric antigen receptor (CAR) or portion thereof. In embodiment 146 provided herein is the composition of embodiment 145, wherein the CAR or portion thereof comprises a polypeptide that binds at least one of B7H3, BCMA, GPRC5D, CD8, CD8a, CD19, CD20, CD22, CD28, 4-1BB, or CD3zeta. In embodiment 147 provided herein is the composition of embodiment 146, wherein the CAR or portion thereof comprises a polypeptide at least 60, at least 70, at least 80, at least 90, at least 95, at least 99%, or 100% identical to any one of the amino acid sequences of SEQ ID NOs: 86-124. In embodiment 148 provided herein is the composition of embodiment 145, wherein the CAR or portion thereof comprises a polypeptide that binds at least one of B7H3, BCMxA, GPRC5D, CD8, CD8a, CD20, CD22, CD28, 4-1BB, or CD3zeta. In embodiment 149 provided herein is the composition of embodiment 148, wherein the CAR or portion thereof comprises a polypeptide at least 60, at least 70, at least 80, at least 90, at least 95, at least 99%, or 100% identical to any one of the amino acid sequences of SEQ ID NOs: 86-104 or 116-124. In embodiment 150 provided herein is the composition of any one of embodiments 112 to 149, wherein the nucleic acid-guided nuclease comprises an engineered, non-naturally occurring nuclease. In embodiment 151 provided herein is the composition of any one of embodiments 112 to 150, wherein the nucleic acid-guided nuclease comprises a Class 1 or a Class 2 nuclease. In embodiment 152 provided herein is the composition of embodiment 151, wherein the nucleic acid-guided nuclease comprises a Type II or a Type V nuclease. In embodiment 153 provided herein is the composition of embodiment 152, wherein the nucleic acid-guided nuclease comprises a Type V-A, V-B, V-C, V-D, or V-E nuclease. In embodiment 154 provided herein is the composition of embodiment 153, wherein the nucleic acid-guided nuclease comprises a Type V-A nuclease. In embodiment 155 provided herein is the composition of embodiment 154, wherein the nucleic acid-guided nuclease comprises a MAD nuclease, an ART nuclease, or an ABW nuclease. In embodiment 156 provided herein is the composition of embodiment 155, wherein the nucleic acid-guided nuclease comprises an amino acid sequence at least 80%, at least 85%, at least 90%, at least 95%, at least 99%, or 100% identical to an amino acid sequence of a MAD, ART, or ABW nuclease. In embodiment 157 provided herein is the composition of embodiment 155, wherein the nucleic acid-guided nuclease comprises a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20 nuclease. In embodiment 158 provided herein is the composition of embodiment 155, wherein the nucleic acid-guided nuclease comprises an ART1, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35 nuclease. In embodiment 159 provided herein is the composition of embodiment 155, wherein the nucleic acid-guided nuclease comprises an amino acid sequence at least 80%, at least 85%, at least 90%, at least 95%, at least 99%, or 100% identical, to the amino acid sequence of MAD2, MAD7, ART2, ART11, or ART11*. In embodiment 160 provided herein is the composition of embodiment 155, wherein the nucleic acid-guided nuclease comprises an amino acid sequence that is at least 80%, at least 85%, at least 90%, at least 95%, 99%, or 100% identical, to the amino acid sequence of SEQ ID NO: 37. In embodiment 161 provided herein is the composition of any one of embodiments 150 to 160, wherein the nucleic acid-guided nuclease further comprises at least one nuclear localization signal (NLS), at least one purification tag, and/or at least one cleavage site. In embodiment 162 provided herein is the composition of embodiment 161, wherein the nucleic acid-guided nuclease comprises at least 4 nuclear localization signals (NLS). In embodiment 163 provided herein is the composition of embodiment 162, wherein the nucleic acid-guided nuclease comprises one N-terminal and three C-terminal nuclease localization signals (NLS). In embodiment 164 provided herein is the composition of embodiment 161 through 163, wherein the nuclear localization signals comprise any one of SEQ ID NOs: 40-56. In embodiment 165 provided herein is the composition of embodiment 164, wherein the NLS comprises SEQ ID NOs: 40, 51, and 56. In embodiment 166 provided herein is the composition of any one of embodiments 112 to 165, wherein the guide nucleic acid comprises (i) a targeter nucleic acid comprising a targeter stem sequence and the spacer sequence, and (ii) a modulator nucleic acid comprising a modulator stem sequence complementary to the targeter stem sequence, and, optionally, a 5′ sequence. In embodiment 167 provided herein is the composition of embodiment 166, wherein the guide nucleic acid comprises a single polynucleotide. In embodiment 168 provided herein is the composition of embodiment 166 or embodiment 167, wherein the guide nucleic acid comprises an engineered, non-naturally occurring guide nucleic acid. In embodiment 169 provided herein is the composition of embodiment 166 or embodiment 168, wherein the targeter nucleic acid and the modulator nucleic acid are separate polynucleotides. In embodiment 170 provided herein is the composition of embodiment 169, wherein the dual guide nucleic acid is capable of binding to and activating a nucleic acid-guided nuclease, that, in a naturally occurring system, is activated by a single crRNA in the absence of a tracrRNA. In embodiment 171 provided herein is the composition of any one of embodiments 112 through 170, wherein the guide nucleic acid further comprises a donor template recruiting sequence. In embodiment 172 provided herein is the composition of any one of embodiments 112 through 171, wherein the target nucleotide sequence is within at least 10, 20, 30, 40, or 50 nucleotides of a protospacer adjacent motif (PAM) that is recognized by the nucleic acid-guided nuclease. In embodiment 173 provided herein is the composition of any one of embodiments 166 through 172, wherein the guide nucleic acid comprises a spacer sequence at least 80%, at least 85%, at least 90%, at least 95%, at least 99.5%, or 100% identical to any one of any one of SEQ ID NOs: 125-2019. In embodiment 174 provided herein is the composition of any one of embodiments 112 through 173, wherein some or all of the guide nucleic acid comprises RNA. In embodiment 175 provided herein is the composition of embodiment 174, wherein at least 50%, at least 70%, at least 90%, at least 95%, or 100% of the guide nucleic acid comprises RNA. In embodiment 176 provided herein is the composition of any one of embodiments 112 through 175, wherein the guide nucleic acid comprises one or more chemical modifications to one or more nucleotides and/or internucleotide linkages at or near the 5′ end, at or near the 3′ end, and/or both. In embodiment 177 provided herein is the composition of embodiment 176, wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, or a combination thereof. In embodiment 178 provided herein is the composition of any one of embodiments 112 through 177, wherein the donor template comprises single-stranded DNA, linear single-stranded RNA, linear double-stranded DNA, linear double-stranded RNA, circular single-stranded DNA, circular single-stranded RNA, circular double-stranded DNA, or circular double-stranded RNA. In embodiment 179 provided herein is the composition of any one of embodiments 114 through 118, 125 through 129, 136 through 140, or 145 through 149, wherein the donor template comprises two homology arms. In embodiment 180 provided herein is the composition of embodiment 179, wherein the homology arms comprise at least 50, 100, 150, 200, 250, 300, 350, 400, 450, 500, 600, 700, 800, or 900 and/or at most 100, 150, 200, 250, 300, 350, 400, 450, 500, 600, 700, 800, 900, or 1000 nucleotides, for example 50-1000 nucleotides, preferably 100-800 nucleotides, more preferably 250-750 nucleotides, even more preferably 400-600 nucleotides. In embodiment 181 provided herein is the composition of any one of embodiments 114 through 118, 125 through 129, 136 through 140, or 145 through 149, wherein the donor template comprises a mutation in a PAM sequence to partially or completely abolish binding of the RNP to the DNA. In embodiment 182 provided herein is the composition of any one of embodiments 114 through 118, 125 through 129, 136 through 140, or 145 through 149, wherein the donor template comprises one or more promoters. In embodiment 183 provided herein is the composition of embodiment 182, wherein the promoter shares at least 70%, at least 75%, at least 80%, at least 85%, at least 90%, at least 95%, at least 99.5%, or 100% sequence identity with any one of SEQ ID NOs: 78-85. In embodiment 184 provided herein is the composition of any one of embodiments 114 through 118, 125 through 129, 136 through 140, or 145 through 149, wherein the donor template comprises one or more chemical modifications to one or more nucleotides and/or internucleotide linkages at or near the 5′ end, at or near the 3′ end, or both. In embodiment 185 provided herein is the composition of embodiment 184, wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, a suitable alternative, or a combination thereof. In embodiment 186 provided herein is the composition of any one of embodiments 112 through 185, wherein the cell comprises an immune cell or a stem cell. In embodiment 187 provided herein is the composition of embodiment 186, wherein the cell comprises an immune cell comprising a neutrophil, cosinophil, basophil, mast cell, monocyte, macrophage, dendritic cell, natural killer cell, or a lymphocyte. In embodiment 188 provided herein is the composition of embodiment 186, wherein the cell comprises a T cell. In embodiment 189 provided herein is the composition of embodiment 186, wherein the cell comprises a stem cell comprising a human pluripotent, multipotent stem cell, embryonic stem cell, induced pluripotent stem cell, hematopoietic stem cell, or a CD34+ cell. In embodiment 190 provided herein is the composition of embodiment 186, wherein the cell comprises a stem cell comprising an iPSC.

In embodiment 191 provided herein is a composition comprising (a) a first guide nucleic acid comprising a spacer sequence complementary to a target nucleotide sequence within a B2M gene, (b) a second guide nucleic acid comprising a spacer sequence complementary to a target nucleotide sequence within a CIITA gene, (c) a third guide nucleic acid comprising a spacer sequence complementary to a target nucleotide sequence within a TCR subunit gene, and (d) one or more nucleic acid-guided nucleases optionally complexed with one or more of the guide nucleic acids of (a), (b), or (c). In embodiment 192 provided herein is the composition of embodiment 191, wherein the gene coding for a subunit of a TCR is a TRAC gene. In embodiment 193 provided herein is the composition of embodiment 191 or 192, wherein the one or more nucleic acid-guided nucleases comprise Class 1 or a Class 2 nucleases. In embodiment 194 provided herein is the composition of embodiment 193, wherein the one or more nucleic acid-guided nucleases comprise Type II or a Type V nuclease. In embodiment 195 provided herein is the composition of embodiment 193, wherein the one or more nucleic acid-guided nucleases comprise Type V-A, V-B, V-C, V-D, or V-E nucleases. In embodiment 196 provided herein is the composition of embodiment 193, wherein the one or more nucleic acid-guided nucleases comprise Type V-A nucleases. In embodiment 197 provided herein is the composition of embodiment 193, wherein the one or more nucleic acid-guided nucleases comprise a MAD nuclease, an ART nuclease, or an ABW nuclease. In embodiment 198 provided herein is the composition of embodiment 193, wherein the one or more nucleic acid-guided nucleases each comprise an amino acid sequence at least 80%, at least 85%, at least 90%, at least 95%, or at least 99% identical to the amino acid sequence of MAD, ART, or ABW nuclease. In embodiment 199 provided herein is the composition of embodiment 193, wherein the one or more nucleic acid-guided nucleases each comprise a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20 nuclease. In embodiment 200 provided herein is the composition of embodiment 193, wherein the one or more nucleic acid-guided nucleases each comprise an ARTI, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35 nuclease. In embodiment 201 provided herein is the composition of embodiment 193, wherein the one or nucleic acid-guided nucleases each comprise an amino acid sequence at least 80%, at least 85%, at least 90%, at least 95%, or at least 99% identical to the amino acid sequence of MAD2, MAD7, ART2, ART11, or ART11*. In embodiment 202 provided herein is the composition of any one of embodiments 191 through 201, wherein the first, second, and/or third guide nucleic acids comprise (i) a targeter nucleic acid comprising a targeter stem sequence and a spacer sequence, and (ii) a modulator nucleic acid comprising a modulator stem sequence complementary to the targeter stem sequence, and, optionally, a 5′ sequence. In embodiment 203 provided herein is the composition of embodiment 202, wherein the targeter nucleic acid and the modulator nucleic acid comprise a single polynucleotide. In embodiment 204 provided herein is the composition of embodiment 202 or embodiment 203, wherein the guide nucleic acid comprises an engineered, non-naturally occurring guide nucleic acid. In embodiment 205 provided herein is the composition of embodiment 202 or embodiment 204, wherein the guide nucleic acid comprises a dual guide nucleic acid, wherein the targeter nucleic acid and the modulator nucleic acid are separate polynucleotides. In embodiment 206 provided herein is the composition of embodiment 205, wherein the dual guide nucleic acid is capable of binding to and activating a nucleic acid-guided nuclease, that, in a naturally occurring system, is activated by a single crRNA in the absence of a tracrRNA. In embodiment 207 provided herein is the composition of any one of embodiments 202 through 206, wherein the target nucleotide sequence is within at least 10, 20, 30, 40, or 50 nucleotides of a protospacer adjacent motif (PAM) that is recognized by the nucleic acid-guided nuclease. In embodiment 208 provided herein is the composition of any one of embodiments 202 through 207, wherein the guide nucleic acid further comprises a donor template recruiting sequence. In embodiment 209 provided herein is the composition of any one of embodiments 202 through 208, wherein the guide nucleic acid comprises a spacer sequence is at least 80%, at least 85%, at least 90%, at least 95%, at least 99.5%, or 100% identical to any one of any one of SEQ ID NOs: 125-2019. In embodiment 210 provided herein is the composition of any one of embodiments 202 through 209, wherein some or all of the guide nucleic acid is RNA. In embodiment 211 provided herein is the composition of embodiment 210, wherein at least 50%, at least 70%, at least 90%, at least 95%, or 100% of the guide nucleic acid comprises RNA. In embodiment 212 provided herein is the composition of any one of embodiments 202 through 211, wherein the guide nucleic acid comprises one or more chemical modifications to one or more nucleotides and/or internucleotide linkages at or near the 5′ end, at or near the 3′ end, and/or both. In embodiment 213 provided herein is the composition of embodiment 212, wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, a suitable alternative, or a combination thereof. In embodiment 214 provided herein is the composition of any one of embodiments 191 to 213, further comprising (e) a first donor template comprising a first transgene. In embodiment 215 provided herein is the composition of embodiment 214, wherein the first transgene comprises a polynucleotide encoding a fusion protein comprising B2M and HLA-A, -B, -C, -D, -E, -F, or -G. In embodiment 216 provided herein is the composition of embodiment 215, wherein the fusion protein comprises HLA-C, -E, or -G. In embodiment 217 provided herein is the composition of embodiment 216, wherein the fusion protein comprises HLA-E or HLA-G. In embodiment 218 provided herein is the composition of embodiment 217, wherein the fusion protein comprises HLA-E. In embodiment 219 provided herein is the composition of embodiment 217, wherein the fusion protein comprises HLA-G. In embodiment 220 provided herein is the composition of any one of embodiments 214 to 219, wherein the first donor template comprises homology arms, wherein the first homology arm is complementary to a region upstream and the second homology arm is complementary to a region downstream of a cleavage site within a B2M gene. In embodiment 221 provided herein is the composition of any one of embodiments 191 through 220, further comprising (f) a second donor template comprising a second transgene. In embodiment 222 provided herein is the composition of embodiment 221, wherein the second transgene comprises a first portion of a polynucleotide coding for a first chimeric antigen receptor (CAR). In embodiment 223 provided herein is the composition of embodiment 222, wherein the CAR or portion thereof comprises a polypeptide that binds at least one of B7H3, BCMA, GPRC5D, CD8, CD8a, CD19, CD20, CD22, CD28, 4-1BB, or CD3zeta. In embodiment 224 provided herein is the composition of embodiment 223, wherein the CAR or portion thereof comprises a polypeptide at least 60, at least 70, at least 80, at least 90, at least 95, at least 99%, or 100% identical to any one of the amino acid sequences of SEQ ID NOs: 86-124. In embodiment 225 provided herein is the composition of embodiment 221, wherein the CAR or portion thereof comprises a polypeptide that binds at least one of B7H3, BCMA, GPRC5D, CD8, CD8a, CD20, CD22, CD28, 4-1BB, or CD3zeta. In embodiment 226 provided herein is the composition of embodiment 225, wherein the CAR or portion thereof comprises a polypeptide at least 60, at least 70, at least 80, at least 90, at least 95, at least 99%, or 100% identical to any one of the amino acid sequences of SEQ ID NOs: 86-104 or 116-124. In embodiment 227 provided herein is the composition of any one of embodiments 222 through 226, further comprising a second portion of the polynucleotide, wherein the second portion codes for a second CAR or portion thereof, different from the first CAR or portion thereof. In embodiment 228 provided herein is the composition of any one of embodiments 221 to 227, wherein the second donor template comprises homology arms, wherein the first homology arm is complementary to a region upstream and the second homology arm is complementary to a region downstream of a cleavage site within a TRC subunit gene. In embodiment 229 provided herein is the composition of any one of embodiments 191 through 228, further comprising (g) a third donor template comprising a third transgene. In embodiment 230 provided herein is the composition of any one of embodiments 214 to 229, wherein the donor template comprises single-stranded DNA, linear single-stranded RNA, linear double-stranded DNA, linear double-stranded RNA, circular single-stranded DNA, circular single-stranded RNA, circular double-stranded DNA, or circular double-stranded RNA. In embodiment 231 provided herein is the composition of any one of embodiments 214 to 230, wherein the donor template comprises a mutation in a PAM sequence to partially or completely abolish binding of the RNP to the DNA. In embodiment 232 provided herein is the composition of any one of embodiments 214 to 231, wherein the donor template comprises one or more promoters. In embodiment 233 provided herein is the composition of embodiment 232, wherein the promoter shares at least 70%, at least 75%, at least 80%, at least 85%, at least 90%, at least 95%, at least 99.5% sequence identity with any one of SEQ ID NOs: 78-85. In embodiment 234 provided herein is the composition of any one of embodiments 214 to 233, wherein the donor template comprises one or more chemical modifications to one or more nucleotides and/or internucleotide linkages at or near the 5′ end, at or near the 3′ end, or both In embodiment 235 provided herein is the composition of embodiment 234, wherein the chemical modification comprises a 2′-O-alkyl, a 2′-O-methyl, a phosphorothioate, a phosphonoacetate, a thiophosphonoacctate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, a suitable alternative, or a combination thereof.

In embodiment 236 provided herein is a modified cell that (a) partially or completely lacks cell surface-expressed (i) active HLA-1 protein, (ii) active HLA-2 protein, or (iii) active TCR protein, and (b) comprises one or more (i) CAR proteins expressed on the cell surface and (ii) fusion proteins comprising HLA-E or HLA-G expressed on the cell surface. In embodiment 237 provided herein is the modified cell of 236, wherein the cell comprises a human cell. In embodiment 238 provided herein is the modified cell of 237, wherein the human cell comprises an immune cell or a stem cell. In embodiment 239 provided herein is the modified cell of 238, wherein the immune cell comprises a neutrophil, cosinophil, basophil, mast cell, monocyte, macrophage, dendritic cell, natural killer cell, or a lymphocyte. In embodiment 240 provided herein is the modified cell of 238, wherein the immune cell comprises a T cell. In embodiment 241 provided herein is the modified cell of 238, wherein the stem cell comprises a human pluripotent, multipotent stem cell, embryonic stem cell, induced pluripotent stem cell, hematopoietic stem cell, CD34+ cell.

In embodiment 242 provided herein is a human cell comprising (a) a first, and optionally a second and/or third nucleic acid-guided nuclease, wherein at least one of the nucleases comprises a CRISPR endonuclease, and (b) at least one of (i) a first guide nucleic acid directed at a first target nucleotide sequence in a gene coding for a subunit of an HLA-1 protein, (ii) a second guide nucleic acid directed at a second target nucleotide sequence in a gene coding for a subunit of an HLA-2 protein or a transcription factor for one or more genes coding for a subunit of an HLA-2 protein, and (iii) a third guide nucleic acid directed at a third target nucleotide sequence coding for a subunit of a TCR. In embodiment 243 provided herein is the human cell of embodiment 242, further comprising (c) a donor template comprising a polynucleotide coding for a chimeric antigen receptor (CAR) protein or part of a CAR. In embodiment 244 provided herein is the human cell of embodiment 243, wherein the protein comprises a protein directed at B7H3, BCMA, GPRC5D, CD19, CD20, CD22, or a combination thereof. In embodiment 245 provided herein is the human cell of embodiment 244, wherein the CAR or portion thereof comprises a polypeptide at least 60, at least 70, at least 80, at least 90, at least 95, at least 99%, or 100% identical to any one of the amino acid sequences of SEQ ID NOS: 86-124. In embodiment 246 provided herein is the human cell of any one of embodiments 243 through 245, wherein the donor template comprises homology arms for insertion at a cleavage site in the subunit of the TCR to which the guide nucleic acid is directed. In embodiment 247 provided herein is the human cell of any one of embodiments 242 to 243, further comprising (d) a donor template comprising a polynucleotide coding an HLA-A, HLA-B, HLA-C, HLA-D, HLA-E, HLA-F, or HLA-G protein. In embodiment 248 provided herein is the human cell of any one of embodiments 242 to 247, wherein the human cell comprises an immune cell or a stem cell. In embodiment 249 provided herein is the human cell of embodiment 248, wherein the human cell comprises an immune cell comprising a neutrophil, cosinophil, basophil, mast cell, monocyte, macrophage, dendritic cell, natural killer cell, or a lymphocyte. In embodiment 250 provided herein is the human cell of embodiment 248, wherein the human cell comprises an immune cell comprising a T cell. In embodiment 251 provided herein is the human cell of embodiment 248, wherein human cell comprises a stem cell comprising a human pluripotent, multipotent stem cell, embryonic stem cell, induced pluripotent stem cell, hematopoietic stem cell, CD34+ cell. In embodiment 252 provided herein is the human cell of embodiment 251, wherein human cell comprises a stem cell comprising an induced pluripotent stem cell.

In embodiment 253 provided herein is a modified human cell comprising (a) reduced or eliminated B2M and knock-in of HLA-E or HLA-G or (b) reduced or eliminated TCR and knock-in. In embodiment 254 provided herein is the modified human cell of embodiment 253, wherein the human cell comprises an immune cell or a stem cell. In embodiment 255 provided herein is the modified human cell of 254, wherein the human cell comprises an immune cell comprising a neutrophil, cosinophil, basophil, mast cell, monocyte, macrophage, dendritic cell, natural killer cell, or a lymphocyte. In embodiment 256 provided herein is the modified human cell of 254, wherein the human cell comprises an immune cell comprising a T cell. In embodiment 257 provided herein is the modified human cell of 254, wherein the human cell comprises a stem cell comprising a human pluripotent, multipotent stem cell, embryonic stem cell, induced pluripotent stem cell, hematopoietic stem cell, CD34+ cell. In embodiment 258 provided herein is the modified human cell of 254, wherein the human cell comprises an induced pluripotent stem cell.

In embodiment 259 provided herein is a human stem cell comprising (a) a first genomic modification in an endogenous B2M gene that partially or completely eliminates expression of the endogenous B2M, (b) a second genomic modification in a CIITA gene that partially or completely eliminates expression of the CIITA, and (c) a third genomic modification in a TCR subunit gene that partially or completely eliminates expression of the TCR subunit. In embodiment 260 provided herein is the human stem cell of embodiment 259, wherein the cell comprises an iPSC. In embodiment 261 provided herein is the human stem cell of embodiment 259 or 260, further comprising (d) an exogenous polynucleotide encoding for a fusion protein comprising one or more HLA-A, -B, -C, -D, -E, -F, or -G protein inserted into the B2M gene. In embodiment 262 provided herein is the human stem cell of any of embodiments 259 to 261, further comprising (c) an exogenous polynucleotide encoding for one or more CARs inserted into the TCR subunit gene. In embodiment 263 provided herein is the human stem cell of embodiment 262, wherein the CAR or portion thereof comprises a polypeptide that binds at least one of BCMA, GPRC5D, CD8, CD8a, CD19, CD20, CD22, CD28, 4-1BB, or CD3zeta.

In embodiment 264 provided herein is a method for treating a disorder comprising administering to an individual suffering from a disorder an effective amount of a composition comprising a composition of any one of the embodiments 1 through 190 or 236 through 263.

In embodiment 265 provided herein is a method of producing a non-immunogenic CAR T cell comprising (a) modifying a genome of a cell to reduce or eliminate cell surface expression of active HLA-1 proteins in the cell and its progeny, (b) introducing into the genome of the cell or one or more of its progeny a first polynucleotide coding for surface expression of a first CAR or portion thereof specific for a first antigen, and (c) introducing into the genome of the cell or one or more of its progeny a second polynucleotide coding for surface expression of a second CAR or portion thereof specific for a second antigen. In embodiment 266 provided herein is the method of embodiment 265, wherein modifying genome of a cell to reduce or eliminate cell surface expression of active HLA-1 proteins comprises introducing a genomic modification into a B2M gene that partially or completely inactivates the B2M gene. In embodiment 267 provided herein is the method of embodiment 266, wherein modifying the genome comprises introducing a substitution, an insertion, a deletion, a nonsense mutation, or a truncation. In embodiment 268 provided herein is the method of embodiment 267, wherein the genomic modification comprises inserting a first transgene into a site within the B2M gene, wherein the first transgene codes for a B2M-HLA subunit fusion protein. In embodiment 269 provided herein is the method of embodiment 268, wherein the HLA subunit of the B2M-HLA subunit fusion protein comprises an HLA-C, -E, or -G subunit. In embodiment 270 provided herein is the method of embodiment 268, wherein the HLA subunit of the B2M-HLA subunit fusion protein comprises an HLA-E or -G subunit. In embodiment 271 provided herein is the method of embodiment 268, wherein the HLA subunit of the B2M-HLA subunit fusion protein comprises an HLA-E. In embodiment 272 provided herein is the method of embodiment 268, wherein the HLA subunit of the B2M-HLA subunit fusion protein comprises an HLA-G. In embodiment 273 provided herein is the method of any one of embodiments 265 through 272, wherein the first and/or second CAR or portion thereof comprises a CAR or portion thereof that binds B7H3, BCMA, GPRC5D, CD8, CD8a, CD19, CD20, CD22, CD28, 4-1BB, or CD3zeta. In embodiment 274 provided herein is the method of embodiment 273, wherein the CAR or portion thereof comprises a polypeptide at least 60, at least 70, at least 80, at least 90, at least 95, at least 99%, or 100% identical to any one of the amino acid sequences of SEQ ID NOs: 86-124. In embodiment 275 provided herein is the method of any one of embodiments 265 through 272, wherein the first and/or second CAR or portion thereof comprises a CAR or portion thereof that binds B7H3, BCMA, GPRC5D, CD8, CD8a, CD20, CD22, CD28, 4-1BB, or CD3zeta. In embodiment 276 provided herein is the method of embodiment 275, wherein the CAR or portion thereof comprises a polypeptide at least 60, at least 70, at least 80, at least 90, at least 95, at least 99%, or 100% identical to any one of the amino acid sequences of SEQ ID NOs: 86-104 or 116-124. In embodiment 277 provided herein is the method of any one of embodiments 265 through 276, wherein the polynucleotide coding for surface expression of a CAR is introduced at a site with a TCR subunit gene or a safe harbor site. In embodiment 278 provided herein is the method of any one of embodiments 265 through 277, further comprising (d) modifying the genome of the cell or one of its progeny to reduce or eliminate cell surface expression of one or more subunits of an HLA-2 protein. In embodiment 279 provided herein is the method of embodiment 278, wherein modifying a genome of the cell or one of its progeny to reduce or eliminate cell surface expression of one or more subunits of an HLA-2 protein comprises introducing a genomic modification into a gene coding for a transcription factor for one or more genes encoding the one or more subunits of an HLA-2 protein that partially or completely inactivates the gene for the transcription factor. In embodiment 280 provided herein is the method of embodiment 279, wherein the genomic modification comprises a substitution, an insertion, a deletion, a nonsense mutation, or a truncation. In embodiment 281 provided herein is the method of embodiment 279 or embodiment 280, wherein the transcription factor comprises CIITA. In embodiment 282 provided herein is the method of any one of embodiments 268 to 281, wherein introducing into the genome comprises delivering into the cell a nucleic acid-guided nuclease system, or one or more polynucleotides encoding for one or more parts of the system, comprising (i) a nucleic acid-guided nuclease and (ii) a guide nucleic acid compatible with and capable of binding to and activating the nucleic acid-guided nuclease, wherein the guide nucleic acid comprises (1) a targeter nucleic acid comprising a targeter stem sequence and a spacer sequence, wherein the spacer sequence is complementary to a target nucleotide sequence within a target polynucleotide of a genome of a human target cell and (2) a modulator nucleic acid comprising a modulator stem sequence complementary to the targeter stem sequence, and, optionally, a 5′ sequence, wherein the nucleic acid-guided nuclease system target and cleave at least one strand in the target polynucleotide at or near the target nucleotide sequence. In embodiment 283 provided herein is the method of embodiment 282, wherein the nucleic acid-guided nuclease comprises a Class 1 or a Class 2 nuclease. In embodiment 284 provided herein is the method of embodiment 283, wherein the nucleic acid-guided nuclease comprises a Type II or a Type V nuclease. In embodiment 285 provided herein is the method of embodiment 284, wherein the nucleic acid-guided nuclease comprises a Type V-A, V-B, V-C, V-D, or V-E nuclease. In embodiment 286 provided herein is the method of embodiment 285, wherein the nucleic acid-guided nuclease comprises a Type V-A nuclease. In embodiment 287 provided herein is the method of embodiment 286, wherein the nucleic acid-guided nuclease comprises a MAD nuclease, an ART nuclease, or an ABW nuclease. In embodiment 288 provided herein is the method of embodiment 286, wherein the nucleic acid-guided nuclease comprises an amino acid sequence at least 80%, at least 85%, at least 90%, at least 95%, at least 99%, or 100% identical to the amino acid sequence of MAD, ART, or ABW nuclease. In embodiment 289 provided herein is the method of embodiment 286, wherein the nucleic acid-guided nuclease comprises a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20 nuclease. In embodiment 290 provided herein is the method of embodiment 286, wherein the nucleic acid-guided nuclease comprises an ART1, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35 nuclease. In embodiment 291 provided herein is the method of embodiment 286, wherein the nucleic acid-guided nuclease comprises an amino acid sequence at least 80%, at least 85%, at least 90%, at least 95%, at least 99%, or 100% identical to the amino acid sequence of MAD2, MAD7, ART2, ART11, or ART11*. In embodiment 292 provided herein is the method of embodiment 286, wherein the nucleic acid-guided nuclease comprises an amino acid sequence that is at least 80%, at least 85%, at least 90%, at least 95%, 99%, or 100% identical, to the amino acid sequence of SEQ ID NO: 37. In embodiment 293 provided herein is the method of any one of embodiments 282 through 292, wherein the nucleic acid-guided nuclease comprises at least one nuclear localization signal (NLS), at least one purification tag, or at least one cleavage site. In embodiment 294 provided herein is the method of embodiment 293, wherein the nucleic acid-guided nuclease comprises at least 4 NLS. In embodiment 295 provided herein is the method of embodiment 294, wherein the nucleic acid-guided nuclease comprises one N-terminal and three C-terminal nuclease localization signals (NLS). In embodiment 296 provided herein is the method of any one of embodiments 293 through 295, wherein the nuclear localization signals comprise any one of SEQ ID NOs: 40-56. In embodiment 297 provided herein is the method of embodiment 296, wherein the NLS comprises SEQ ID NOs: 40, 51, and 56. In embodiment 298 provided herein is the method of embodiment 282 through 297, wherein the guide nucleic acid comprises a single polynucleotide. In embodiment 299 provided herein is the method of embodiment 282 through 297, wherein the guide nucleic acid comprises a dual guide nucleic acid, wherein the targeter nucleic acid and the modulator nucleic acid are separate polynucleotides. In embodiment 300 provided herein is the method of embodiment 299, wherein the dual guide nucleic acid is capable of binding to and activating a nucleic acid-guided nuclease, that, in a naturally occurring system, is activated by a single crRNA in the absence of a tracrRNA. In embodiment 301 provided herein is the method of embodiment 282 through 300, wherein the target nucleotide sequence is within at least 10, at least 20, at least 30, at least 40, or at least 50 nucleotides of a protospacer adjacent motif (PAM) that is recognized by a nuclease with which the guide nucleic acid is compatible. In embodiment 302 provided herein is the method of embodiment 282 through 301, wherein the guide nucleic acid and the nuclease form a nucleic acid-guided nuclease complex. In embodiment 303 provided herein is the method of embodiment 302, wherein the guide nucleic acid further comprises a donor template recruiting sequence. In embodiment 304 provided herein is the method of embodiment 282 through 303, wherein the guide nucleic acid comprises a spacer sequence is at least 80%, at least 85%, at least 90%, at least 95%, at least 99.5%, or 100% identical to any one of any one of SEQ ID NOs: 125-2019. In embodiment 305 provided herein is the method of embodiment 282 through 304, wherein some or all of the guide nucleic acid is RNA. In embodiment 306 provided herein is the method of embodiment 305, wherein at least 50%, at least 70%, at least 90%, at least 95%, or 100% of the guide nucleic acid comprises RNA. In embodiment 307 provided herein is the method of embodiment 282 through 306, wherein the guide nucleic acid comprises one or more chemical modifications to one or more nucleotides and/or internucleotide linkages at or near the 5′ end, at or near the 3′ end, and/or both. In embodiment 308 provided herein is the method of embodiment 307, wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, a suitable alternative, or a combination thereof. In embodiment 309 provided herein is the method of embodiment 282 through 308, wherein introducing into the genome further comprises delivering a donor template comprising the transgene. In embodiment 310 provided herein is the method of embodiment 309, wherein the donor template comprises two homology arms flanking the transgene. In embodiment 311 provided herein is the method of embodiment 310, wherein the homology arms comprise at most 1000, at most 900, at most 800, at most 700, at most 600, at most 500 nucleotides. In embodiment 312 provided herein is the method of any one of embodiments 309 through 311, wherein the donor template comprises single-stranded DNA, linear single-stranded RNA, linear double-stranded DNA, linear double-stranded RNA, circular single-stranded DNA, circular single-stranded RNA, circular double-stranded DNA, or circular double-stranded RNA. In embodiment 313 provided herein is the method of any one of embodiments 309 through 312, wherein the donor template comprises a mutation in a PAM sequence to partially or completely abolish binding of the RNP to the DNA. In embodiment 314 provided herein is the method of any one of embodiments 309 through 313, wherein the donor template comprises one or more promoters. In embodiment 315 provided herein is the method of embodiment 314, wherein the promoter shares at least 70%, at least 75%, at least 80%, at least 85%, at least 90%, at least 95%, at least 99.5%, or 100% sequence identity with any one of SEQ ID NOs: 78-85. In embodiment 316 provided herein is the method of any one of embodiments 309 through 315, wherein the donor template comprises one or more chemical modifications to one or more nucleotides and/or internucleotide linkages at or near the 5′ end, at or near the 3′ end, and/or both. In embodiment 317 provided herein is the method of embodiment 316, wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, a suitable alternative, or a combination thereof. In embodiment 318 provided herein is the method of any one of embodiments 309 through 317, wherein at least portion of the donor template is inserted by an innate cell repair mechanism at or near the strand break. In embodiment 319 provided herein is the method of embodiment 318, wherein the innate cell repair mechanism comprises homology directed repair (HDR). In embodiment 320 provided herein is the method of any one of embodiments 265 to 319, wherein the cell comprises a human cell. In embodiment 321 provided herein is the method of embodiment 320, wherein the human cell comprises an immune cell or a stem cell. In embodiment 322 provided herein is the method of embodiment 321, wherein the human cell comprises an immune cell comprising a neutrophil, cosinophil, basophil, mast cell, monocyte, macrophage, dendritic cell, natural killer cell, or a lymphocyte. In embodiment 323 provided herein is the method of embodiment 321, wherein the human cell comprises an immune cell comprising a T cell. In embodiment 324 provided herein is the method of embodiment 321, wherein the human cell comprises a stem cell comprising a human pluripotent, multipotent stem cell, embryonic stem cell, induced pluripotent stem cell, hematopoietic stem cell, CD34+ cell. In embodiment 325 provided herein is the method of embodiment 321, wherein the human cell comprises a stem cell comprising an induced pluripotent stem cell. In embodiment 326 provided herein is the method of any one of embodiments 268 to 325, wherein delivering comprises electroporation.

In embodiment 327 provided herein is a method for producing a population of non-immunogenic CAR T cells comprising (a) modifying a genome of a first cell to reduce or eliminate cell surface expression of HLA-1 proteins in the first cell and its progeny, (b) introducing into the genome of the first cell a first polynucleotide coding for surface expression of a first CAR specific for a first antigen on the first cell, (c) modifying a genome of a second cell to reduce or eliminate cell surface expression of HLA-1 proteins in the second cell and its progeny, and (d) introducing into the genome of the second cell a second polynucleotide coding for surface expression of a second CAR specific for a second antigen on the second cell, wherein the first and second cells are the same cell, the first cell is a progeny of the second cell, or the second cell is a progeny of the first cell.

In embodiment 328 provided herein is a method of producing a cell with an engineered genome comprising (a) modifying a B2M gene in the genome of a first cell to reduce or eliminate expression of the B2M gene, (b) modifying a T cell receptor (TCR) subunit gene in the genome of a second cell to reduce or eliminate expression of the subunit, (c) modifying a CIITA gene in the genome of a third cell to reduce or eliminate expression of the CIITA gene, and (d) introducing a first transgene into the genome of a fourth cell, wherein the first transgene codes for a B2M-HLA subunit fusion protein. In embodiment 329 provided herein is the method of embodiment 328, wherein (a) through (d) are performed simultaneously, wherein the first, second, third, and fourth cells are the same cell. In embodiment 330 provided herein is the method of embodiment 328, wherein one or more of (a) through (d) are performed sequentially. In embodiment 331 provided herein is the method of embodiment 330, wherein one or more cells resulting from embodiment 330 are propagated prior to performing the remainder of (a) through (d) not performed in embodiment 330. In embodiment 332 provided herein is the method of any one of embodiments 328 through 331, wherein the TCR subunit comprises an alpha subunit or a beta subunit or a TRAC, TRBC, CD3E, CD3D, CD3G, or CD3Z protein. In embodiment 333 provided herein is the method of embodiment 332, wherein the TCR subunit comprises an alpha subunit. In embodiment 334 provided herein is the method of any one of embodiments 328 to 333, wherein the HLA subunit of the B2M-HLA subunit fusion protein comprises an HLA-C, -E, or -G subunit. In embodiment 335 provided herein is the method of embodiment 334, wherein the HLA subunit of the B2M-HLA subunit fusion protein comprises an HLA-E or -G subunit. In embodiment 336 provided herein is the method of embodiment 334, wherein the HLA subunit of the B2M-HLA subunit fusion protein comprises an HLA-E. In embodiment 337 provided herein is the method of embodiment 334, wherein the HLA subunit of the B2M-HLA subunit fusion protein comprises an HLA-G. In embodiment 338 provided herein is the method of any one of embodiments 328 to 337, wherein the first transgene is introduced at a site within the B2M gene. In embodiment 339 provided herein is the method of any one of embodiments 328 to 338, wherein the cell comprises a human cell. In embodiment 340 provided herein is the method of embodiment 339, wherein the human cell comprises an immune cell or a stem cell. In embodiment 341 provided herein is the method of embodiment 340, wherein the human cell comprises an immune cell comprising a neutrophil, cosinophil, basophil, mast cell, monocyte, macrophage, dendritic cell, natural killer cell, or a lymphocyte. In embodiment 342 provided herein is the method of embodiment 340, wherein the human cell comprises an immune cell comprising a T cell. In embodiment 343 provided herein is the method of embodiment 340, wherein the human cell comprises a stem cell comprising a human pluripotent, multipotent stem cell, embryonic stem cell, induced pluripotent stem cell, hematopoietic stem cell, CD34+ cell. In embodiment 344 provided herein is the method of embodiment 340, wherein the human cell comprises a stem cell comprising an induced pluripotent stem cell. In embodiment 345 provided herein is the method of any one of embodiments 328 to 344, further comprising (c) introducing a second transgene into the genome, wherein the second transgene codes for a chimeric antigen receptor (CAR) or portion thereof. In embodiment 346 provided herein is the method of embodiment 345, wherein the second transgene is introduced at a site within the TCR subunit gene. In embodiment 347 provided herein is the method of any one of embodiments 345 to 346, wherein the CAR or portion thereof comprises polypeptide that binds to B7H3, BCMA, GPRC5D, CD8, CD8a, CD19, CD20, CD22, CD28, 4-1BB, or CD3zeta. In embodiment 348 provided herein is the method of embodiment 347, wherein the CAR or portion thereof comprises a polypeptide at least 60, at least 70, at least 80, at least 90, at least 95, at least 99%, or 100% identical to any one of the amino acid sequences of SEQ ID NOs: 86-124. In embodiment 349 provided herein is the method of any one of embodiments 345 to 346, wherein the CAR or portion thereof comprises a polypeptide that binds at least one of B7H3, BCMA, GPRC5D, CD8, CD8a, CD20, CD22, CD28, 4-1BB, or CD3zeta. In embodiment 350 provided herein is the method of embodiment 349, wherein the CAR or portion thereof comprises a polypeptide at least 60, at least 70, at least 80, at least 90, at least 95, at least 99%, or 100% identical to any one of the amino acid sequences of SEQ ID NOs: 86-104 or 116-124. In embodiment 351 provided herein is the method of any one of embodiments 328 to 350, wherein the modifying of step (a) comprises contacting DNA of the genome with a first nucleic acid-guided nuclease complexed with a first compatible guide nucleic acid (gNA) targeted to a first target nucleotide sequence within the B2M gene so that the DNA is cleaved at or near the first target nucleotide sequence. In embodiment 352 provided herein is the method of any one of embodiments 328 to 351, wherein the modifying of step (b) comprises contacting DNA of the genome with a second nucleic acid-guided nuclease complexed with a second compatible guide nucleic acid targeted to a second target nucleotide sequence within the TCR subunit gene so that the DNA is cleaved at or near the second target nucleotide sequence. In embodiment 353 provided herein is the method of anyone of embodiments 328 to 352, wherein the modifying of step (c) comprises contacting DNA of the genome with a third nucleic acid-guided nuclease complexed with a third compatible guide nucleic acid targeted to a third target nucleotide sequence within the CIITA subunit gene so that the DNA is cleaved at or near the third target nucleotide sequence.

In embodiment 354 provided herein is a method of modifying a genome of a human cell comprising (a) modifying a B2M gene in the genome to reduce or eliminate expression of the B2M gene, (b) modifying a T cell receptor (TCR) subunit gene in the genome to reduce or eliminate expression of the subunit, and (c) modifying a CIITA gene in the genome to reduce or eliminate expression of the CIITA gene, wherein at least 2 of (a) to (c) are performed sequentially, not simultaneously, thereby producing a modified human cell.

In embodiment 355 provided herein is a composition comprising a modified human cell comprising: (a) a first genomic modification comprising a first portion of a first polynucleotide, wherein the first portion comprises a transgene, inserted into a site with a TRC subunit gene, whereby the TRC subunit gene is partially or completely inactivated and the transgene is expressed; and (b) a second genomic modification comprising a second polynucleotide coding for a fusion protein of B2M and HLA-E or HLA-G inserted into a B2M gene, whereby endogenous B2M is partially or completely inactivated and the fusion protein is expressed. In embodiment 356 provided herein is the composition of claim 355, wherein the TRC subunit gene is completely inactivated. In embodiment 357 provided herein is the composition of claim 355 or claim 356, wherein the endogenous B2M gene is completely inactivated. In embodiment 358 provided herein is the composition of claim 355, further comprising: (c) a third genomic modification in a CIITA gene, wherein the CIITA gene is partially or completely inactivated. In embodiment 359 provided herein is the composition of claim 358, wherein the CIITA gene is completely inactivated. In embodiment 360 provided herein is the composition of any one of claims 355-359, wherein the TRC subunit gene comprises a TRAC, TRBC, CD3E, CD3D, CD3G, or CD3Z gene. In embodiment 361 provided herein is the composition of claim 360, wherein the TRC subunit gene comprises a TRAC gene. In embodiment 362 provided herein is the composition of claim 360, wherein the TRC subunit gene comprises a TRBC gene. In embodiment 363 provided herein is the composition of claim 360, wherein the TRC subunit gene comprises a CD3E gene. In embodiment 364 provided herein is the composition of claim 360, wherein the TRC subunit gene comprises a CD3D gene. In embodiment 365 provided herein is the composition of claim 360, wherein the TRC subunit gene comprises a CD3G gene. In embodiment 366 provided herein is the composition of claim 360,
wherein the TRC subunit gene comprises a CD3Z gene. In embodiment 367 provided herein is the composition of any one of claims 355-366, wherein the transgene comprises a CAR or portion thereof, a cytokine, and/or a reporter gene. In embodiment 368 provided herein is the composition of claim 367, wherein the transgene comprises a CAR or portion thereof.

In embodiment 369 provided herein is a composition comprising a modified human cell comprising: (a) a first genomic modification comprising a first portion of a polynucleotide, wherein the first portion comprises a transgene, inserted into a site with a TRC subunit gene, whereby the TRC subunit gene is partially or completely inactivated and the transgene is expressed; and (b) a second genomic modification in a CIITA gene, wherein the CIITA gene is partially or completely inactivated. In embodiment 370 provided herein is the composition of claim 369, wherein the TRC subunit gene is completely inactivated. In embodiment 371 provided herein is the composition of claim 369 or claim 356, wherein the CIITA gene is completely inactivated. In embodiment 372 provided herein is the composition of any one of claims 369-371, further comprising: (c) a third genomic modification comprising a polynucleotide coding for a fusion protein of B2M and HLA-E or HLA-G inserted into a B2M gene, whereby endogenous B2M is partially or completely inactivated and the fusion protein is expressed. In embodiment 373 provided herein is the composition of claim 372, wherein endogenous B2M is completely inactivated. In embodiment 374 provided herein is the composition of any one of claims 369-373, wherein the TRC subunit gene comprises a TRAC, TRBC, CD3E, CD3D, CD3G, or CD3Z gene. In embodiment 375 provided herein is the composition of claim 374, wherein the TRC subunit gene comprises a TRAC gene. In embodiment 376 provided herein is the composition of claim 374, wherein the TRC subunit gene comprises a TRBC gene. In embodiment 377 provided herein is the composition of claim 374, wherein the TRC subunit gene comprises a CD3E gene. In embodiment 378 provided herein is the composition of claim 374, wherein the TRC subunit gene comprises a CD3D gene. In embodiment 379 provided herein is the composition of claim 374, wherein the TRC subunit gene comprises a CD3G gene. In embodiment 380 provided herein is the composition of claim 374, wherein the TRC subunit gene comprises a CD3Z gene. In embodiment 381 provided herein is the composition of any one of claims 369-380, wherein the transgene comprises a CAR or portion thereof, a cytokine, and/or a reporter gene. In embodiment 382 provided herein is the composition of claim 381, wherein the transgene comprises a CAR or portion thereof.

In embodiment 383 provided herein is a composition comprising a modified human cell comprising: (a) a first genomic modification comprising a polynucleotide coding for a fusion protein of B2M and HLA-E or HLA-G inserted into a B2M gene, whereby endogenous B2M is partially or completely inactivated and the fusion protein is expressed; (b) a second genomic modification in a CIITA gene, wherein the CIITA gene is partially or completely inactivated; and (c) a third genomic modification comprising a first portion of a polynucleotide, wherein the first portion comprises a transgene, inserted into a site with a TRC subunit gene, whereby the TRC subunit gene is partially or completely inactivated and the transgene is expressed. In embodiment 384 provided herein is the composition of claim 383, wherein endogenous B2M is completely inactivated. In embodiment 385 provided herein is the composition of claim 383 or claim 384, wherein the CIITA gene is completely inactivated. In embodiment 386 provided herein is the composition of any one of claims 383-385, wherein the TRC subunit gene is completely inactivated. In embodiment 387 provided herein is the composition of any one of claims 383-386, wherein the TRC subunit gene comprises a TRAC, TRBC, CD3E, CD3D, CD3G, or CD3Z gene. In embodiment 388 provided herein is the composition of claim 387, wherein the TRC subunit gene comprises a TRAC gene. In embodiment 389 provided herein is the composition of claim 387, wherein the TRC subunit gene comprises a TRBC gene. In embodiment 390 provided herein is the composition of claim 387, wherein the TRC subunit gene comprises a CD3E gene. In embodiment 391 provided herein is the composition of claim 387, wherein the TRC subunit gene comprises a CD3D gene. In embodiment 392 provided herein is the composition of claim 387, wherein the TRC subunit gene comprises a CD3G gene. In embodiment 393 provided herein is the composition of claim 387, wherein the TRC subunit gene comprises a CD3Z gene. In embodiment 394 provided herein is the composition of any one of claims 383-393, wherein the transgene comprises a CAR or portion thereof, a cytokine, and/or a reporter gene. In embodiment 395 provided herein is the composition of claim 394, wherein the transgene comprises a CAR or portion thereof.

### VIII. EXAMPLES

### A. Example 1

This example demonstrates successful triple knock out of TCR, HLA-I, and HLA-II with and without CAR insertion into the TRAC locus using multiplexed editing with RNPs comprising either a single gRNA or a gRNA comprising a targeter and a modulator nucleic acid.

Primary human pan T-cells were isolated from whole leukopaks, processed on the day of receipt, and CD3-positive pan T-cells were separated from other peripheral blood mononuclear cells. Cells were characterized by flow cytometry before and after negative selection for viability, CD3 expression, and CD4/CD8 positivity. Cells were gated for proper size/shape, and singlets were selected. Cells displayed >98% viability prior to and following enrichment for pan T-cells, and the negative selection strategy resulted in enrichment of CD3 positive cells from 76.8% to 97.0%. Additionally, the CD4: CD8 ratio was maintained through the enrichment. The cells were frozen and used as needed. Viability was measured by imaging in a flow cell with a volume of 1.4 μL using the Nucleocounter NC-200 and Vial cassettes after staining cells Acridine orange and DAPI to differentiate live cells (acridine orange positive cells) from dead cells (DAPI positive cells).

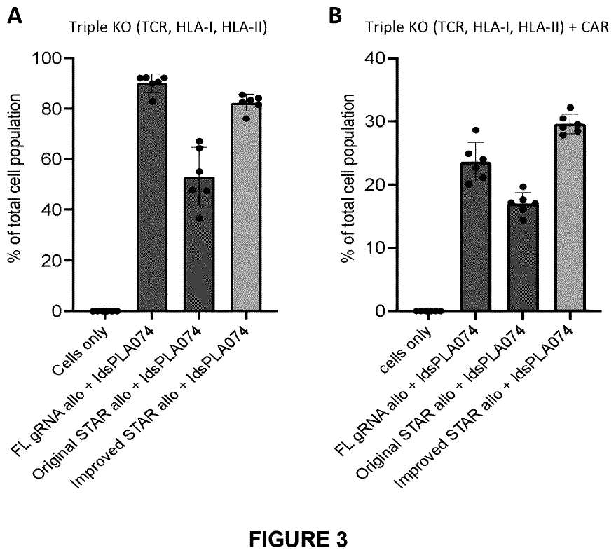

Primary human pan T-cell specific nucleofection conditions, including nucleofection buffer, nucleofection program (EO-115), and IL-2 concentration (200 IU/mL), were obtained from recommendations by Lonza and Nucleofection solution. 8-12% CAR expression for each of the two CARs was observed (FIGS. 3A and B; 2^{nd} and 3^{rd} bars for single (FL gRNA) and dual (STAR) gRNAs respectively). To obtain higher insertion rates, additional optimization on the protocol using nucleofection program EH-115 and increasing the IL-2 concentration to 500 IU in post-nucleofection cell culturing was performed. Furthermore, inclusion of a ssODN in the nucleofection reaction increased delivery of the gene-editing reagents in primary human pan T-cells. Specifically, inclusion of a 200 nt ssODN in the nucleofection solution yielded high viability at day 11 post-nucleofection and CAR expression up to 40% when using 1 μg linearized dsDNA (ldsPLA074). Inclusion of an ssODN in the nucleofection insertion protocol consistently produced a CAR expressing cell population between 40-70% of the total cell population at eleven to twelve days post-nucleofection FIGS. 3A and B; fourth bars). Specifically, FIG. 3A shows editing efficiency for three simultaneously genomic modifications comprising triple knock-out (KO) of HLA-1, HLA-2, and TCR as measured by flow cytometry following three treatment conditions: (1) untreated control; (2) treatment with gRNAs comprising a single polynucleotide (FL gRNA) in the presence of linear double stranded DNA (ldsPLA074); (3) treatment with gRNAs comprising a dual guide RNA (STAR) in the presence of linear double stranded DNA; and (4) treatment with gRNAs comprising a dual guide RNA (STAR) in the presence of linear double stranded DNA using improved conditions as described above. Specifically, FIG. 3B shows editing efficiency for three simultaneously genomic modifications comprising triple knock-out (KO) of HLA-1, HLA-2, and TCR as well as insertion of a polynucleotide encoding for a CAR polypeptide as measured as measured by flow cytometry following three treatment conditions: (1) untreated control; (2) treatment with gRNAs comprising a single polynucleotide (FL gRNA) in the presence of linear double stranded DNA (ldsPLA074); (3) treatment with gRNAs comprising a dual guide RNA (STAR) in the presence of linear double stranded DNA; and (4) treatment with gRNAs comprising a dual guide RNA (STAR) in the presence of linear double stranded DNA using improved conditions as described above.

### B. Example 2

This example demonstrates reduction of surface-expressed TCR through knockout of CD3D.

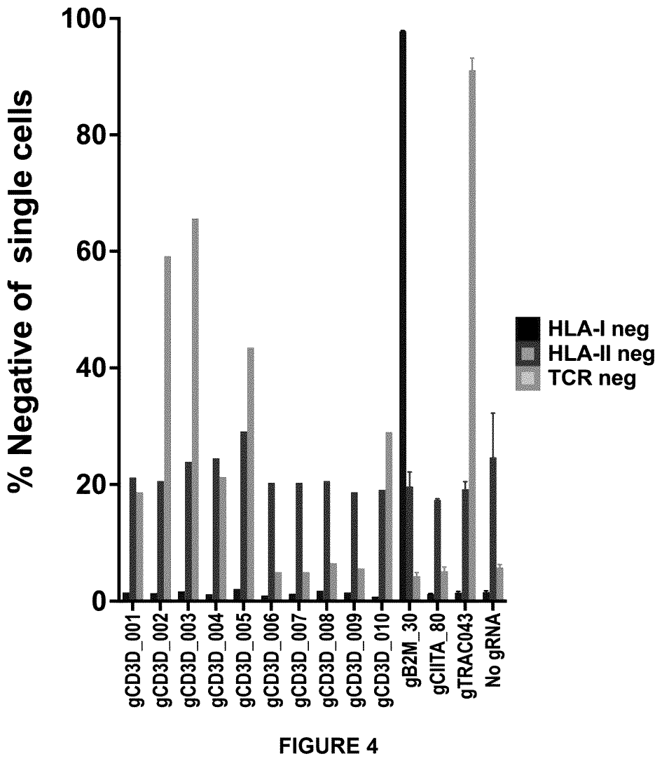

Primary human pan T-cells were transfected 100pmol RNPs complexed with either gCD3D_001 (spacer sequence listed as SEQ ID NO: 655), gCD3D_002 (spacer sequence listed as SEQ ID NO: 656), gCD3D_003 (spacer sequence listed as SEQ ID NO: 657), gCD3D_004 (spacer sequence listed as SEQ ID NO: 658), gCD3D_005 (spacer sequence listed as SEQ ID NO: 659), gCD3D_006 (spacer sequence listed as SEQ ID NO: 660), gCD3D_007 (spacer sequence listed as SEQ ID NO: 661), gCD3D_008 (spacer sequence listed as SEQ ID NO: 662), gCD3D_009 (spacer sequence listed as SEQ ID NO: 663), gCD3D_010 (spacer sequence listed as SEQ ID NO: 664), gB2M30 (spacer sequence listed as SEQ ID NO: 2012), gCIITA_80 (spacer sequence listed as SEQ ID NO: 2018), gTRAC043 (spacer sequence listed as SEQ ID NO: 1996), or no guide RNA in Nucleofection buffer P3 using nucleofection program EH-115. After transfection, the cells were stained with anti-HLAI, anti-HLAII, and and -TCR antibodies and analyzed by flow cytometry. (FIG. 4). Specifically, FIG. 4 shows percent of negative cells after treatment (y-axis) for each tested gNA for each antibody stain (HLA-I, black; HLA-II dark gray, TCR-light gray).

### C. Example 3

This example demonstrates reduction of surface-expressed TCR through knockout of CD247 and/or CD3G.

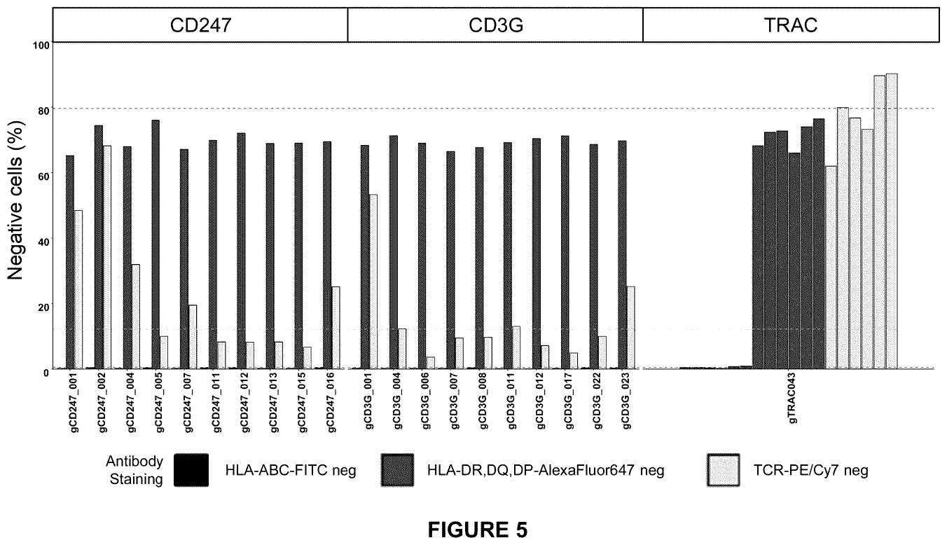

Primary human pan T-cells were transfected 100pmol RNPs complexed with either gCD247_001 (spacer sequence listed as SEQ ID NO: 688), gCD247_002 (spacer sequence listed as SEQ ID NO: 689), gCD247_004 (spacer sequence listed as SEQ ID NO: 691), gCD247_005 (spacer sequence listed as SEQ ID NO: 692), gCD247_007 (spacer sequence listed as SEQ ID NO: 694), gCD247_011 (spacer sequence listed as SEQ ID NO: 698), gCD247_012 (spacer sequence listed as SEQ ID NO: 699), gCD247_013 (spacer sequence listed as SEQ ID NO: 700), gCD247_015 (spacer sequence listed as SEQ ID NO: 702), gCD247_016 (spacer sequence listed as SEQ ID NO: 703), gCD3G_001 (spacer sequence listed as SEQ ID NO: 665), gCD3G_004 (spacer sequence listed as SEQ ID NO: 668), gCD3G_006 (spacer sequence listed as SEQ ID NO: 670), gCD3G_007 (spacer sequence listed as SEQ ID NO: 671), gCD3G_008 (spacer sequence listed as SEQ ID NO: 672), gCD3G_011 (spacer sequence listed as SEQ ID NO: 675), gCD3G_012 (spacer sequence listed as SEQ ID NO: 676), gCD3G_017 (spacer sequence listed as SEQ ID NO: 681), gCD3G_022 (spacer sequence listed as SEQ ID NO: 686), gCD3G_023 (spacer sequence listed as SEQ ID NO: 687), gTRAC043 (spacer sequence listed as SEQ ID NO: 1996), or no guide RNA in Nucleofection buffer P3 using nucleofection program EH-115. Reduced TCR surface expression was observed with gCD247_001, gCD247_002, gCD247_004, gCD247_016, gCD3G_001 and gCD247_023 (FIG. 5). Specifically, FIG. 5 shows percent of negative cells after treatment (y-axis) for each tested gNA for each antibody stain (HLA-I, black; HLA-II dark gray, TCR-light gray).

### D. Example 4

This example demonstrates success knockout of TCR with or without simultaneous knock in of a CAAR polypeptide.

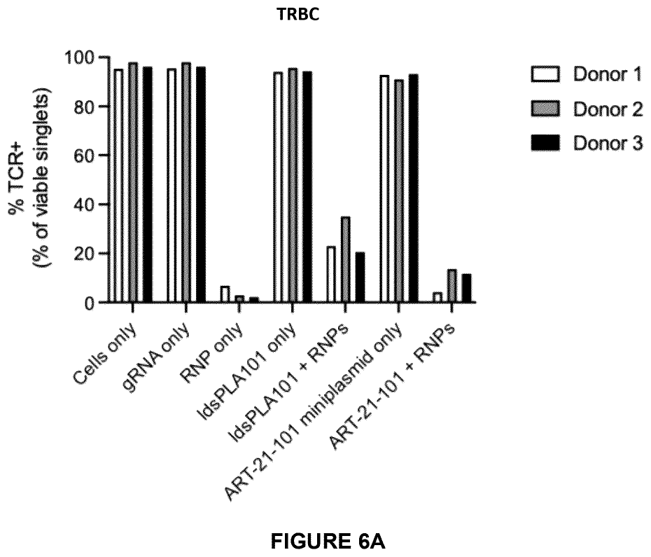

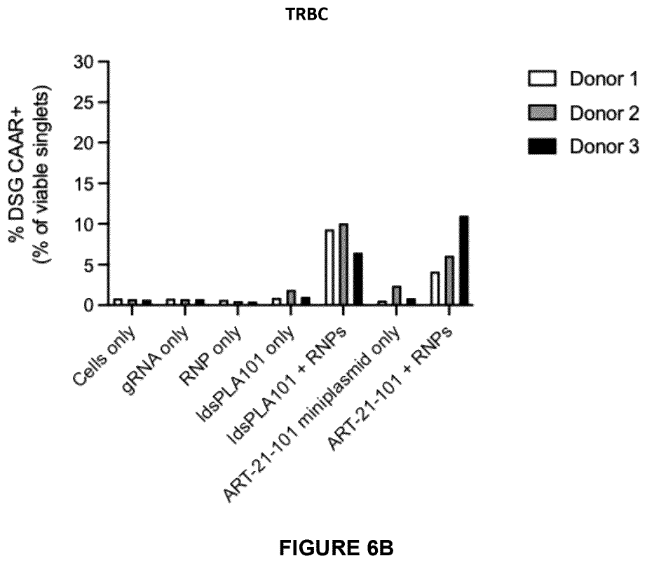

Primary human pan T-cells were transfected 100pmol RNPs complexed with either gTRBC1_2_003 (spacer sequence listed as SEQ ID NO: 2000) or no guide RNA in Nucleofection buffer P3 using nucleofection program EH-115. For knock in experiments, cells were simultaneously transfected with ART-21-101 miniplasmid comprising the CAAR. FIG. 6 demonstrates editing efficiency for TRBC without and with KI of a polynucleotide encoding for a CAAR polypeptide as measured by flow cytometry (anti-TCR, anti-CAAR staining): (column 1) untreated control; (column 2) treatment with gRNA without the presence of polypeptide comprising a nuclease, (column 3) treatment with gRNA and a CRISPR nuclease (RNPs), (column 4) a linearized polynucleotide, (column 5) a linearized polynucleotide encoding a CAAR polypeptide and RNPs, (column 6) a circular polynucleotide, and (column 7) a circular polynucleotide encoding a CAAR polypeptide and RNPs. Substantial TCR KO (y-axis) was observed in the samples when the RNPs were present (columns 3 (RNP only), 5 (ldsPLA101 only), and 7 (ART-210191+RNPs)) (FIG. 6A). CAAR expression (y-axis) was observed in the cells that were transfected with the RNPs and the linearized or circular polynucleotide encoding the CAAR polypeptide (5 (ldsPLA101 only) and 7 (ART-210191+RNPs)) (FIG. 6B).

ART-21-101 miniplasmid sequence:
(SEQ ID NO: 2048)
CGCGCACCCACACCCAGGCCAGGGTGTTGTC
CGGCACCACCTGGTCCTGGACCGCGCTGATGAACAGGGTCACGTCGTCCCGGACCACACCGGCGA
AGTCGTCCTCCACGAAGTCCCGGGAGAACCCGAGCCGGTCGGTCCAGAACTCGACCGCTCCGGCG
ACGTCGCGCGCGGTGAGCACCGGAACGGCACTGGTCAACTTGGCCATACTCTTCCTTTTTCAATA
TTATTGAAGCATTTATCAGGGTTATTGTCTCATGAGCGGATACATAACGCGTTTAGAGTCTCTCA
GCTGGTACACGAAGCTTAATGCCAACATACCATAAACCTCCCATTCTGCTAATGCCCAGCCTAAG
TTGGGGAGACCACTCCAGATTCCAAGATGTACAGTTTGCTTTGCTGGGCCTTTTTCCCATGCCTG
CCTTTACTCTGCCAGAGTTATATTGCTGGGGTTTTGAAGAAGATCCTATTAAATAAAAGAATAAG
CAGTATTATTAAGTAGCCCTGCATTTCAGGTTTCCTTGAGTGGCAGGCCAGGCCTGGCCGTGAAC
GTTCACTGAAATCATGGCCTCTTGGCCAAGATTGATAGCTTGTGCCTGTCCCTGAGTCCCAGTCC
ATCACGAGCAGCTGGTTTCTAAGATGCTATTTCCCGTATAAAGCATGAGACCGTGACTTGCCAGC
CCCACAGAGCCCCGCCCTTGTCCATCACTGGCATCTGGACTCCAGCCTGGGTTGGGGCAAAGAGG
GAAATGAGATCATGTCCTAACCCTGATCCTCTTGTCCCACAGATATCCAGAACCCTGACCCTGCC
GTGGGCAGCGGCGCTACTAACTTCAGCCTGCTGAAGCAGGCTGGCGACGTGGAGGAGAACCCTGG
ACCTATGCTGCTGCTGGTGACATCCCTGCTGCTGTGCGAACTGCCTCATCCCGCTTTCCTGCTGA
TTCCTGAAGTCCAGCTGGTCGAGAGCGGAGGAGGACTGGTGCAGCCTGGAGGATCACTGAGACTG
AGCTGCGCCGCTTCCGGATTCACCTTTAGCTCCTTCGGCATGCACTGGGTGAGGCAGGCACCAGG
AAAAGGCCTGGAGTGGGTCGCTTACATCTCTAGTGACTCAAGCGCCATCTACTATGCAGATACCG
TGAAAGGCAGGTTTACAATCAGTCGCGACAACGCTAAGAATTCCCTGTATCTGCAGATGAACTCT
CTGCGCGACGAGGATACAGCAGTCTACTATTGCGGGGGGGGAAGAGAAAATATCTACTATGGAAG
CCGACTGGACTACTGGGGACAGGGAACCACAGTGACAGTCTCCTCTGGAGGAGGAGGAAGCGGAG
GAGGAGGATCCGGAGGAGGCGGGTCTGATATCCAGCTGACTCAGAGCCCCTCCTTCCTGTCTGCC
AGTGTGGGCGACAGGGTCACTATTACCTGTAAGGCATCCCAGAACGTGGATACCAATGTCGCCTG
GTACCAGCAGAAGCCCGGGAAAGCACCTAAGGCCCTGATCTATTCAGCCAGCTACCGATATTCTG
GCGTGCCAAGTCGGTTCTCCGGATCTGGCAGTGGGACTGACTTTACACTGACTATTAGTTCACTG
CAGCCCGAAGATTTTGCTACCTACTATTGTCAGCAGTACAATAACTACCCATTCACCTTCGGACA
GGGGACAAAACTGGAAATCAAAGAAAGCAAGTACGGACCGCCCTGCCCCCCTTGCCCTGGCCAGC
CTAGAGAACCCCAGGTGTACACCCTGCCTCCCAGCCAGGAAGAGATGACCAAGAACCAGGTGTCC
CTGACCTGCCTGGTCAAAGGCTTCTACCCCAGCGATATCGCCGTGGAATGGGAGAGCAACGGCCA
GCCCGAGAACAACTACAAGACCACCCCCCCTGTGCTGGACAGCGACGGCAGCTTCTTCCTGTACT
CCCGGCTGACCGTGGACAAGAGCCGGTGGCAGGAAGGCAACGTCTTCAGCTGCAGCGTGATGCAC
GAGGCCCTGCACAACCACTACACCCAGAAGTCCCTGAGCCTGAGCCTGGGCAAGATGTTCTGGGT
GCTGGTGGTGGTCGGAGGCGTGCTGGCCTGCTACAGCCTGCTGGTCACCGTGGCCTTCATCATCT
TTTGGGTGAAACGGGGCAGAAAGAAACTCCTGTATATATTCAAACAACCATTTATGAGACCAGTA
CAAACTACTCAAGAGGAAGATGGCTGTAGCTGCCGATTTCCAGAAGAAGAAGAAGGAGGATGTGA
ACTGCGGGTGAAGTTCAGCAGAAGCGCCGACGCCCCTGCCTACCAGCAGGGCCAGAATCAGCTGT
ACAACGAGCTGAACCTGGGCAGAAGGGAAGAGTACGACGTCCTGGATAAGCGGAGAGGCCGGGAC
CCTGAGATGGGCGGCAAGCCTCGGCGGAAGAACCCCCAGGAAGGCCTGTATAACGAACTGCAGAA
AGACAAGATGGCCGAGGCCTACAGCGAGATCGGCATGAAGGGCGAGCGGAGGCGGGGCAAGGGCC
ACGACGGCCTGTATCAGGGCCTGTCCACCGCCACCAAGGATACCTACGACGCCCTGCACATGCAG
GCCCTGCCCCCAAGGGCTAGCGGCAGTGGAGAGGGCAGAGGAAGTCTGCTAACATGCGGTGACGT
CGAGGAGAATCCTGGCCCAATGGAAGATTTTAACATGGAGAGTGACAGCTTTGAAGATTTCTGGA
AAGGTGAAGATCTTAGTAATTACAGTTACAGCTCTACCCTGCCCCCTTTTCTACTAGATGCCGCC
CCATGTGAACCAGAATCCCTGGAAATCAACAAGTATTTTGTGGTCATTATCTATGCCCTGGTATT
CCTGCTGAGCCTGCTGGGAAACTCCCTCGTGATGCTGGTCATCTTATACAGCAGGGTCGGCCGCT
CCGTCACTGATGTCTACCTGCTGAACCTAGCCTTGGCCGACCTACTCTTTGCCCTGACCTTGCCC
ATCTGGGCCGCCTCCAAGGTGAATGGCTGGATTTTTGGCACATTCCTGTGCAAGGTGGTCTCACT
CCTGAAGGAAGTCAACTTCTATAGTGGCATCCTGCTACTGGCCTGCATCAGTGTGGACCGTTACC
TGGCCATTGTCCATGCCACACGCACACTGACCCAGAAGCGCTACTTGGTCAAATTCATATGTCTC
AGCATCTGGGGTCTGTCCTTGCTCCTGGCCCTGCCTGTCTTACTTTTCCGAAGGACCGTCTACTC
ATCCAATGTTAGCCCAGCCTGCTATGAGGACATGGGCAACAATACAGCAAACTGGCGGATGCTGT
TACGGATCCTGCCCCAGTCCTTTGGCTTCATCGTGCCACTGCTGATCATGCTGTTCTGCTACGGA
TTCACCCTGCGTACGCTGTTTAAGGCCCACATGGGGCAGAAGCACCGGGCCATGCGGGTCATCTT
TGCTGTCGTCCTCATCTTCCTGCTCTGCTGGCTGCCCTACAACCTGGTCCTGCTGGCAGACACCC
TCATGAGGACCCAGGTGATCCAGGAGACCTGTGAGCGCCGCAATCACATCGACCGGGCTCTGGAT
GCCACCGAGATTCTGGGCATCCTTCACAGCTGCCTCAACCCCCTCATCTACGCCTTCATTGGCCA
GAAGTTTCGCCATGGACTCCTCAAGATTCTAGCTATACATGGCTTGATCAGCAAGGACTCCCTGC
CCAAAGACAGCAGGCCTTCCTTTGTTGGCTCTTCTTCAGGGCACACTTCCACTACTCTCTAACTG
TGCCTTCTAGTTGCCAGCCATCTGTTGTTTGCCCCTCCCCCGTGCCTTCCTTGACCCTGGAAGGT
GCCACTCCCACTGTCCTTTCCTAATAAAATGAGGAAATTGCATCGCATTGTCTGAGTAGGTGTCA
TTCTATTCTGGGGGGTGGGGTGGGGCAGGACAGCAAGGGGGAGGATTGGGAAGACAATAGCAGGC
ATGCTGGGGATACCAGCTGAGAGACTCTAATTCCAGTGACAAGTCTGTCTGCCTATTCACCGATT
TTGATTCTCAAACAAATGTGTCACAAAGTAAGGATTCTGATGTGTATATCACAGACAAAACTGTG
CTAGACATGAGGTCTATGGACTTCAAGAGCAACAGTGCTGTGGCCTGGAGCAACAAATCTGACTT
TGCATGTGCAAACGCCTTCAACAACAGCATTATTCCAGAAGACACCTTCTTCCCCAGCCCAGGTA
AGGGCAGCTTTGGTGCCTTCGCAGGCTGTTTCCTTGCTTCAGGAATGGCCAGGTTCTGCCCAGAG
CTCTGGTCAATGATGTCTAAAACTCCTCTGATTGGTGGTCTCGGCCTTATCCATTGCCACCAAAA
CCCTCTTTTTACTAAGAAACAGTGAGCCTTGTTCTGGCAGTCCAGAGAATGACACGGGAAAAAAG
CAGATGAAGAGAAGGTGGCAGGAGAAAGCTTCGTGTACCAGCTGAGAGACTCTAAATCGACTCTA
GAGGATCCCGGGTACCGAGCTCGAATTCGGATATCCTCGAGACTAGTGGGCCCGTTTAAACACAT
GTGTTTTTCCATAGGCTCCGCCCCCCTGACGAGCATCACAAAAATCGACGCTCAAGTCAGAGGTG
GCGAAACCCGACAGGACTATAAAGATACCAGGCGTTTCCCCCTGGAAGCTCCCTCGTGCGCTCTC
CTGTTCCGACCCTGCCGCTTACCGGATACCTGTCCGCCTTTCTCCCTTCGGGAAGCGTGGCGCTT
TCTCATAGCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAGTCCAACC
CGGTAAGACACGACTTATCGCCACTGGCAGCAGCCACTGGTAACAGGATTAGCAGAGCGAGGTAT
GTAGGCGGTGCTACAGAGTTCTTGAAGTGGTGGCCTAACTACGGCTACACTAGAAGGACAGTATT
TGGTATCTGCGCTCTGCTGAAGCCAGTTACCTTCGGAAAAAGAGTTGGTAGCTCTTGATCCGGCA
AACAAACCACCGCTGGTAGCGGTGGTTTTTTTGTTTGCAAGCAGCAGATTACGCGCAGAAAAAAA
GGATCTCAAGAAGATCCTTTGATCTTTTCTACGTCAGTCCTGCTCCTCGGCCACGAAGTGCACGC
AGTTGCCGGCCGGGTCGCGCAGGGCGAACTCCCGCCCCCACGGCTGCTCGCCGATCTCGGTCATG
GCCGGCCCGGAGGCGTCCCGGAAGTTCGTGGACACGACCTCCGACCACTCGGCGTACAGCTCGTC
CAGGC
ldsPLA101 sequence:
(SEQ ID NO: 2049)
ATTGGGATCCTCAGCAAAGGAAAATTATAATTAGAAAAAGTC
AATTTAGTTATTGTAATTATACCACTAATGAGAGTTTCCTACCTCGAGTTTCAGGATTACATAGC
CATGCACCAAGCAAGGCTTTGAAAAATAAAGATACACAGATAAATTATTTGGATAGATGATCAGA
CAAGCCTCAGTAAAAACAGCCAAGACAATCAGGATATAATGTGACCATAGGAAGCTGGGGAGACA
GTAGGCAATGTGCATCCATGGGACAGCATAGAAAGGAGGGGCAAAGTGGAGAGAGAGCAACAGAC
ACTGGGATGGTGACCCCAAAACAATGAGGGCCTAGAATGACATAGTTGTGCTTCATTACGGCCCA
TTCCCAGGGCTCTCTCTCACACACACAGAGCCCCTACCAGAACCAGACAGCTCTCAGAGCAACCC
TGGCTCCAACCCCTCTTCCCTTTCCAGAGGACCTGAACAAGGTGTTCCCACCCGAGGTCGCTGTG
TTTGAGCCATCAGAAGCACGTGAGGCTCCGGTGCCCGTCAGTGGGCAGAGCGCACATCGCCCACA
GTCCCCGAGAAGTTGGGGGGAGGGGTCGGCAATTGAACCGGTGCCTAGAGAAGGTGGCGCGGGGT
AAACTGGGAAAGTGATGTCGTGTACTGGCTCCGCCTTTTTCCCGAGGGTGGGGGAGAACCGTATA
TAAGTGCAGTAGTCGCCGTGAACGTTCTTTTTCGCAACGGGTTTGCCGCCAGAACACAGGTAAGT
GCCGTGTGTGGTTCCCGCGGGCCTGGCCTCTTTACGGGTTATGGCCCTTGCGTGCCTTGAATTAC
TTCCACCTGGCTGCAGTACGTGATTCTTGATCCCGAGCTTCGGGTTGGAAGTGGGTGGGAGAGTT
CGAGGCCTTGCGCTTAAGGAGCCCCTTCGCCTCGTGCTTGAGTTGAGGCCTGGCCTGGGCGCTGG
GGCCGCCGCGTGCGAATCTGGTGGCACCTTCGCGCCTGTCTCGCTGCTTTCGATAAGTCTCTAGC
CATTTAAAATTTTTGATGACCTGCTGCGACGCTTTTTTTCTGGCAAGATAGTCTTGTAAATGCGG
GCCAAGATCTGCACACTGGTATTTCGGTTTTTGGGGCCGCGGGCGGCGACGGGGCCCGTGCGTCC
CAGCGCACATGTTCGGCGAGGCGGGGCCTGCGAGCGCGGCCACCGAGAATCGGACGGGGGTAGTC
TCAAGCTGGCCGGCCTGCTCTGGTGCCTGGCCTCGCGCCGCCGTGTATCGCCCCGCCCTGGGCGG
CAAGGCTGGCCCGGTCGGCACCAGTTGCGTGAGCGGAAAGATGGCCGCTTCCCGGCCCTGCTGCA
GGGAGCTCAAAATGGAGGACGCGGCGCTCGGGAGAGCGGGCGGGTGAGTCACCCACACAAAGGAA
AAGGGCCTTTCCGTCCTCAGCCGTCGCTTCATGTGACTCCACTGAGTACCGGGCGCCGTCCAGGC
ACCTCGATTAGTTCTCGTGCTTTTGGAGTACGTCGTCTTTAGGTTGGGGGGAGGGGTTTTATGCG
ATGGAGTTTCCCCACACTGAGTGGGTGGAGACTGAAGTTAGGCCAGCTTGGCACTTGATGTAATT
CTCCTTGGAATTTGCCCTTTTTGAGTTTGGATCTTGGTTCATTCTCAAGCCTCAGACAGTGGTTC
AAAGTTTTTTTCTTCCATTTCAGGTGTCGTGAGCTAGAGCCACCATGGAGTTTGGGCTGAGCTGG
CTTTTTCTTGTGGCTATTTTAAAAGGTGTCCAGTGCGGATCCGAGCTGCGGATCGAGACAAAGGG
CCAGTACGACGAGGAAGAGATGACAATGCAGCAGGCCAAGCGGCGGCAGAAACGCGAGTGGGTCA
AGTTCGCCAAGCCCTGCAGAGAGGGCGAGGACAACAGCAAGCGGAACCCTATCGCCAAGATCACC
AGCGACTACCAGGCCACCCAGAAGATCACCTACCGGATCAGCGGCGTGGGCATCGACCAGCCCCC
TTTCGGCATCTTCGTGGTGGACAAGAACACCGGCGACATCAACATCACCGCCATCGTGGACAGAG
AGGAAACCCCCAGCTTCCTGATCACCTGTCGGGCCCTGAATGCCCAGGGCCTGGACGTGGAAAAG
CCCCTGATCCTGACCGTGAAGATCCTGGACATCAACGACAACCCCCCCGTGTTCAGCCAGCAGAT
CTTCATGGGCGAGATCGAGGAAAACAGCGCCAGCAACAGCCTCGTGATGATCCTGAACGCCACCG
ACGCCGACGAGCCCAACCACCTGAATAGCAAGATCGCCTTCAAGATCGTGTCCCAGGAACCCGCC
GGAACCCCCATGTTCCTGCTGAGCAGAAATACCGGCGAAGTGCGGACCCTGACCAACAGCCTGGA
TAGAGAGCAGGCCAGCAGCTACCGGCTGGTGGTGTCTGGCGCTGACAAGGATGGCGAGGGCCTGA
GCACACAGTGCGAGTGCAACATCAAAGTGAAGGACGTGAACGACAACTTCCCTATGTTCCGGGAC
AGCCAGTACAGCGCCCGGATCGAAGAGAACATCCTGAGCAGCGAGCTGCTGCGGTTCCAAGTGAC
CGACCTGGACGAAGAGTACACCGACAACTGGCTGGCCGTGTACTTCTTCACCAGCGGCAACGAGG
GCAATTGGTTCGAGATCCAGACCGACCCCCGGACCAATGAGGGCATCCTGAAGGTCGTGAAGGCC
CTGGACTACGAGCAGCTGCAGAGCGTGAAGCTGTCTATCGCCGTGAAGAACAAGGCCGAGTTCCA
CCAGTCCGTGATCAGCCGGTACAGAGTGCAGAGCACCCCCGTGACCATCCAAGTGATCAACGTGC
GCGAGGGCATTGCCTTCGCTAGCGGTGGCGGAGGTTCTGGAGGTGGAGGTTCCTCCGGAATCTAC
ATCTGGGCGCCCTTGGCCGGGACTTGTGGGGTCCTTCTCCTGTCACTGGTTATCACCCTTTACTG
CAAACGGGGCAGAAAGAAACTCCTGTATATATTCAAACAACCATTTATGAGACCAGTACAAACTA
CTCAAGAGGAAGATGGCTGTAGCTGCCGATTTCCAGAAGAAGAAGAAGGAGGATGTGAACTGAGA
GTGAAGTTCAGCAGGAGCGCAGACGCCCCCGCGTACCAGCAGGGCCAGAACCAGCTCTATAACGA
GCTCAATCTAGGACGAAGAGAGGAGTACGATGTTTTGGACAAGAGACGTGGCCGGGACCCTGAGA
TGGGGGGAAAGCCGAGAAGGAAGAACCCTCAGGAAGGCCTGTACAATGAACTGCAGAAAGATAAG
ATGGCGGAGGCCTACAGTGAGATTGGGATGAAAGGCGAGCGCCGGAGGGGCAAGGGGCACGATGG
CCTTTACCAGGGTCTCAGTACAGCCACCAAGGACACCTACGACGCCCTTCACATGCAGGCCCTGC
CCCCTCGCTAAGTCGACAATCAACCTCTGGATTACAAAATTTGTGAAAGATTGACTGGTATTCTT
AACTATGTTGCTCCTTTTACGCTATGTGGATACGCTGCTTTAATGCCTTTGTATCATGCTATTGC
TTCCCGTATGGCTTTCATTTTCTCCTCCTTGTATAAATCCTGGTTGCTGTCTCTTTATGAGGAGT
TGTGGCCCGTTGTCAGGCAACGTGGCGTGGTGTGCACTGTGTTTGCTGACGCAACCCCCACTGGT
TGGGGCATTGCCACCACCTGTCAGCTCCTTTCCGGGACTTTCGCTTTCCCCCTCCCTATTGCCAC
GGCGGAACTCATCGCCGCCTGCCTTGCCCGCTGCTGGACAGGGGCTCGGCTGTTGGGCACTGACA
ATTCCGTGGTGTTGTCGGGGAAGCTGACGTCCTTTCCTTGGCTGCTCGCCTGTGTTGCCACCTGG
ATTCTGCGCGGGACGTCCTTCTGCTACGTCCCTTCGGCCCTCAATCCAGCGGACCTTCCTTCCCG
CGGCCTGCTGCCGGCTCTGCGGCCTCTTCCGCGTCTTCGCCTTCGCCCTCAGACGAGTCGGATCT
CCCTTTGGGCCGCCTCCCCGCCTGCGACTGTGCCTTCTAGTTGCCAGCCATCTGTTGTTTGCCCC
TCCCCCGTGCCTTCCTTGACCCTGGAAGGTGCCACTCCCACTGTCCTTTCCTAATAAAATGAGGA
AATTGCATCGCATTGTCTGAGTAGGTGTCATTCTATTCTGGGGGGTGGGGGGGGCAGGACAGCA
AGGGGGAGGATTGGGAAGACAATAGCAGGCATGCTGGGGATGCGGTGGGCTCTATGGGAGATCTC
CCACACCCAAAAGGCCACACTGGTGTGCCTGGCCACAGGCTTCTTCCCTGACCACGTGGAGCTGA
GCTGGTGGGTGAATGGGAAGGAGGTGCACAGTGGGGTCAGCACGGACCCGCAGCCCCTCAAGGAG
CAGCCCGCCCTCAATGACTCCAGATACTGCCTGAGCAGCCGCCTGAGGGTCTCGGCCACCTTCTG
GCAGAACCCCCGCAACCACTTCCGCTGTCAAGTCCAGTTCTACGGGCTCTCGGAGAATGACGAGT
GGACCCAGGATAGGGCCAAACCCGTCACCCAGATCGTCAGCGCCGAGGCCTGGGGTAGAGCAGGT
GAGTGGGGCCTGGGGAGATGCCTGGAGGAGATTAGGTGAGACCAGCTACCAGGGAAAATGGAAAG
ATCCAGGTAGCAGACAAGACTAGATCCAAAAAGAAAGGAACCAGCGCACACCATGAAGGAGAATT
GGGCACCTGTGGTTCATTCTTCTCCCAGATTCTCAGC

### E. Example 5

This example demonstrates reduction of surface-expressed TCR through knockout of CD3E with or without simultaneous knock in of a CAR.

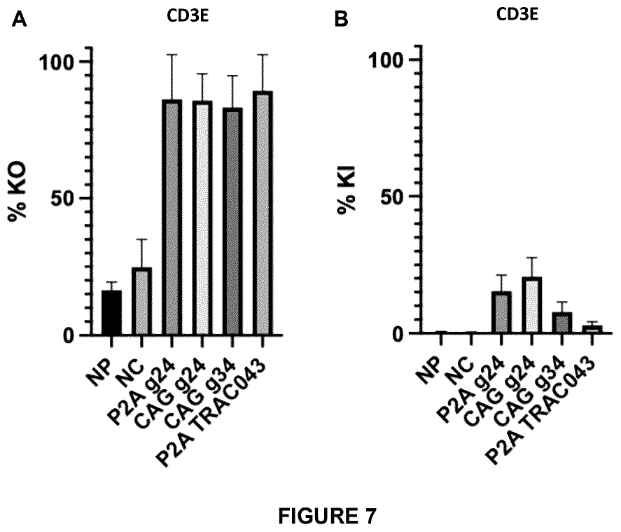

Primary human pan T-cells were transfected 100pmol RNPs complexed with either gCD3E_24 (spacer sequence listed as SEQ ID NO: 2001), gCD3E_34 (spacer sequence listed as SEQ ID NO: 2002), gTRAC043 (spacer sequence listed as SEQ ID NO: 1996), or no guide RNA in Nucleofection buffer P3 using nucleofection program EH-115. For knock in studies, the cells were cotransfected with one of the following repair templates: CD3E_24 P2A miniplasmid, CD3E_24 CAG miniplasmid, CD3E_34 CAG miniplasmid, PLA074-TRAC043 P2A miniplasmid. FIG. 7 demonstrates editing efficiency for CD3E without and with KI of a polynucleotide encoding for a CAR polypeptide as measured by flow cytometry (anti-TCR, anti-CAR staining): (column 1) No program (NP) control, (column 2) no cargo (NC) control, (column 3) treatment with gCD3E_24 RNPs and a circular CD3E_24 P2A miniplasmid repair template, (column 4) treatment with gCD3E_24 RNPs and a circular CD3E_24 CAG miniplasmid repair template, (column 5) treatment with gCD3E_34 RNPs and a circular CD3E_34 CAG miniplasmid, and (column 6) treatment with gTRAC043 RNPs (spacer sequence listed as SEQ ID NO: 1996) and a circular PLA074-TRAC043 P2A miniplasmid repair template (positive control). Substantial TCR KO (y-axis) was observed in the samples when the RNPs were present (columns 3-5) (FIG. 7A). CAR expression (y-axis) was observed in the cells that were transfected with the RNPs and the circular polynucleotide encoding the CAR polypeptide (columns 3-5) (FIG. 7B).

CD3E_24 P2A miniplasmid sequence:
(SEQ ID NO: 2050)
CGCGCACCCACACCCAGGCCAGGGTGTTGTC
CGGCACCACCTGGTCCTGGACCGCGCTGATGAACAGGGTCACGTCGTCCCGGACCACACCGGCGA
AGTCGTCCTCCACGAAGTCCCGGGAGAACCCGAGCCGGTCGGTCCAGAACTCGACCGCTCCGGCG
ACGTCGCGCGCGGTGAGCACCGGAACGGCACTGGTCAACTTGGCCATACTCTTCCTTTTCAATAT
TATTGAAGCATTTATCAGGGTTATTGTCTCATGAGCGGATACATAACGCGTGCCCTCAGTATCCT
GGATCTGAAAATTGGGATCCTCAGCAGACACGTGAGTTTATTGGTCTTTTATTTATGCCCTGTCT
GAGGATGCAGATTGGTGGGTAGATGAGAAGGAACTGATTGAGAGAGATTAACCCCAAGAACTGAT
ATCTTCCCAGCATTGCATTCTCAACTCCATTTTAGAAAGGTTCCAAATAGGGACTTCTGTGGGTT
TTTCTTTACATCCATCTTACCCTTCCCAAGTCCCCATGTCCCTGCGTAAACCCTAAAGCCACCTC
TCAAaaggttctctagttcccttcaaggttctctagttcccttcaTTCCACATATCTCCTCTTCC
ACACCCTCTAGCCAGTAGAGCTCCCTTCTGACAAGCAAGTCTAAGATCTAGATGACAGATGACTT
CCTGCATTTGGGTGGTTCTTTTGTCACTAATTTGCCTTTTCTAAAATTGTCCTGGTTTCTTCTGC
CAATTTCCCTTCTTTCTCCCCAGCATATAAAGTCTCCATCTCTGGAACCACAGTAATATTGACAT
GCCCTGGCAGCGGCGCTACTAACTTCAGCCTGCTGAAGCAGGCTGGCGACGTGGAGGAGAACCCT
GGACCTATGGCTCTCCCAGTGACTGCCCTACTGCTTCCCCTAGCGCTTCTCCTGCATGCAGAGGT
GAAGCTGCAGCAGTCTGGGGCTGAGCTGGTGAGGCCTGGGTCCTCAGTGAAGATTTCCTGCAAGG
CTTCTGGCTATGCATTCAGTAGCTACTGGATGAACTGGGTGAAGCAGAGGCCTGGACAGGGTCTT
GAGTGGATTGGACAGATTTATCCTGGAGATGGTGATACTAACTACAATGGAAAGTTCAAGGGTCA
AGCCACACTGACTGCAGACAAATCCTCCAGCACAGCCTACATGCAGCTCAGCGGCCTAACATCTG
AGGACTCTGCGGTCTATTTCTGTGCAAGAAAGACCATTAGTTCGGTAGTAGATTTCTACTTTGAC
TACTGGGGCCAAGGGACCACGGTCACCGTCTCCTCAGGTGGAGGTGGATCAGGaGGtGGaGGtTC
TGGTGGAGGaGGATCTGACATTGAGCTCACCCAGTCTCCAAAATTCATGTCCACATCAGTAGGAG
ACAGGGTCAGCGTCACCTGCAAGGCCAGTCAGAATGTGGGTACTAATGTAGCCTGGTATCAACAG
AAACCAGGACAATCTCCTAAACCACTGATTTACTCGGCAACCTACCGGAACAGTGGAGTCCCTGA
TCGCTTCACAGGCAGTGGATCTGGGACAGATTTCACTCTCACCATCACTAACGTGCAGTCTAAAG
ACTTGGCAGACTATTTCTGTCAACAATATAACAGGTATCCGTACACGTCCGGAGGGGGGACCAAG
CTGGAGATCAAACGGGCGGCCGCAATTGAAGTTATGTATCCTCCTACTTACCTAGACAATGAGAA
GAGCAATGGAACCATTATCCATGTGAAAGGGAAACACCTTTGTCCAAGTCCCCTATTTCCCGGAC
CTTCTAAGCCCTTTTGGGTGCTGGTGGTGGTTGGTGGAGTCCTGGCTTGCTATAGCTTGCTAGTA
ACAGTGGCCTTTATTATTTTCTGGGTGAGGAGTAAGAGGAGCAGGCTCCTGCACAGTGACTACAT
GAACATGACTCCtCGCCGCCCCGGGCCtACaCGcAAGCATTACCAGCCCTATGCCCCACCACGCG
ACTTCGCAGCCTATCGCTCCAGAGTGAAGTTCAGCAGGAGCGCAGAGCCCCCCGCGTACCAGCAG
GGCCAGAACCAGCTCTATAACGAGCTCAATCTAGGACGAAGAGAGGAGTACGATGTTTTGGACAA
GAGACGTGGCCGGGACCCTGAGATGGGGGGAAAGCCGAGAAGGAAGAACCCTCAGGAAGGCCTGT
ACAATGAACTGCAGAAAGATAAGATGGCGGAGGCCTACAGTGAGATTGGGATGAAAGGCGAGCGC
CGGAGGGGCAAGGGGCACGATGGCCTTTACCAGGGTCTCAGTACAGCCACCAAGGACACCTACGA
CGCCCTTCACATGCAGGCCCTGCCCCCTCGCTAACGACTGTGCCTTCTAGTTGCCAGCCATCTGT
TGTTTGCCCCTCCCCCGTGCCTTCCTTGACCCTGGAAGGTGCCACTCCCACTGTCCTTTCCTAAT
AAAATGAGGAAATTGCATCGCATTGTCTGAGTAGGTGTCATTCTATTCTGGGGGGTGGGGTGGGG
CAGGACAGCAAGGGGGAGGATTGGGAAGACAATAGCAGGCATGCTGGGGATGCGGTGGGCTCTAT
GGCAGTATCCTGGATCTGAAATACTATGGCAACACAatgataaaaacataggcggtgatgaggat
gataaaaacataggcagtgatgaggatCACCTGTCACTGAAGGAATTTTCAGAATTGGAGCAAAG
TGGTTATTATGTCTGCTACCCCAGAGGAAGCAAACCAGAAGATGCGAACTTTTATCTCTACCTGA
GGGCAAGAGGTAATCCAGGTCTCCAGAACAGGTACCACCGGCTCTTTAGGGAGGACCATTCAAAA
GGGCATTCTCAGTGATTTTCCCTAACCCAGCTCACAGTGCCCAGGCGTCTTTGCGCTTCCTCCCA
CACTCAATCCTGGGACTCTCTGGTACCACACGGCATCAGTGTTTTCTGGAATATAGATTAAACAC
CAATATGAGGCTTCTGGGTAACCCCAGTCTGTGCGAGATCTAAAATAGCAACTCCCTAAGAGACA
GGACTGGGTCATTTGCACCGCATCACACCCAGGTTCATAGCACACCAGCGGCCGCTTTCAGATCC
AGGATACTGAGGGCATGTTTTTCCATAGGCTCCGCCaCCCTGACGAGCATCACAAAAATCGACGC
TCAAGTCAGAGGTGGCGAAACCCGACAGGACTATAAAGATACCAGGCGTTTCCCCCTGGACGCTC
CCTCGTGCGCTCTCCTGTTCCGACCCTGCCGCTTACCGGATACCTGTCCGCCTTTCTCCCTTCGG
GAAGCGTGGCGCTTTCTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTTATCCGGTAACTATCG
TCTTGAGTCCAACCCGGTAAGACACGACTTATCGCCACTGGCAGCAGCCACTGGTAACAGGATTA
GCAGAGCGAGGTATGTAGGCGGTGCTACAGAGTTCTTGAAGTGGTGGCCTAACTACGGCTACACT
AGAAGaACAGTATTTGGTATCCGCGCTCTGCTGAAGCCAGTTACCTTCGGAAAAAGAGTTGGTAG
CTCTTGATCCGGCAAACAAACCACCGCTGGTAGCGGTGGTTTTTTTGTTTGCAAGCAGCAGATTA
CGCGCAGgAAAAAAGGATCTCAAGAAGATCCTTTGATCTTTTCTACGTCAGTCCTGCTCCTCGGC
CACGAAGTGCACGCAGTTGCCGGCCGGGTCGCGCAGGGCGAACTCCCGCCCCCACGGCTGCTCGC
CGATCTCGGTCATGGCCGGCCCGGAGGCGTCCCGGAAGTTCGTGGACACGACCTCCGACCACTCG
GCGTACAGCTCGTCCAGGC
CD3E_24 CAG miniplasmid sequence:
(SEQ ID NO: 2051)
TTTCCATAGGCTCCGCCaCCCTGACGAGCA
TCACAAAAATCGACGCTCAAGTCAGAGGTGGCGAAACCCGACAGGACTATAAAGATACCAGGCGT
TTCCCCCTGGAAGCTCCCTCGTGCGCTCTCCTGTTCCGACCCTGCCGCTTACCGGATACCTGTCC
GCCTTTCTCCCTTCGGGAAGCGTGGCGCTTTCTCATAGCTCACGCTGTAGGTATCTCAGTTCGGT
GTAGGTCGTTCGCTCCAAGCTGGGCTGTGTGCACGAACCCCCCGTTCAGCCCGACCGCTGCGCCT
TATCCGGTAACTATCGTCTTGAGTCCAACCCGGTAAGACACGACTTATCGCCACTGGCAGCAGCC
ACTGGTAACAGGATTAGCAGAGCGAGGTATGTAGGCGGTGCTACAGAGTTCTTGAAGTGGTGGCC
TAACTACGGCTACACTAGAAGGACAGTATTTGGTATCTGCGCTCTGCTGAAGCCAGTTACCTTCG
GAAAAAGAGTTGGTAGCTCTTGATCCGGCAAACAAACCACCGCTGGTAGCGGTGGTTTTTTTGTT
TGCAAGCAGCAGATTACGCGCAGAAAAAAAGGATCTCAAGAAGATCCTTTGATCTTTTCTACGTC
AGTCCTGCTCCTCGGCCACGAAGTGCACGCAGTTGCCGGCCGGGTCGCGCAGGGCGAACTCCCGC
CCCCACGGCTGCTCGCCGATCTCGGTCATGGCCGGCCCGGAGGCGTCCCGGAAGTTCGTGGACAC
GACCTCCGACCACTCGGCGTACAGCTCGTCCAGGCCGCGCACCCACACCCAGGCCAGGGTGTTGT
CCGGCACCACCTGGTCCTGGACCGCGCTGATGAACAGGGTCACGTCGTCCCGGACCACACCGGCG
AAGTCGTCCTCCACGAAGTCCCGGGAGAACCCGAGCCGGTCGGTCCAGAACTCGACCGCTCCGGC
GACGTCGCGCGCGGTGAGCACCGGAACGGCACTGGTCAACTTGGCCATACTCTTCCTTTTCAATA
TTATTGAAGCATTTATCAGGGTTATTGTCTCATGAGCGGATACATAACGCGTGCCCTCAGTATCC
TGGATCTGAAAATTGGGATCCTCAGCAGACACGTGAGTTTATTGGTCTTTTATTTATGCCCTGTC
TGAGGATGCAGATTGGTGGGTAGATGAGAAGGAACTGATTGAGAGAGATTAACCCCAAGAACTGA
TATCTTCCCAGCATTGCATTCTCAACTCCATTTTAGAAAGGTTCCAAATAGGGACTTCTGTGGGT
TTTTCTTTACATCCATCTTACCCTTCCCAAGTCCCCATGTCCCTGCGTAAACCCTAAAGCCACCT
CTCAAaaggttctctagttcccttcaaggttctctagttcccttcaTTCCACATATCTCCTCTTC
CACACCCTCTAGCCAGTAGAGCTCCCTTCTGACAAGCAAGTCTAAGATCTAGATGACAGATGACT
TCCTGCATTTGGGTGGTTCTTTTGTCACTAATTTGCCTTTTCTAAAATTGTCCTGGTTTCTTCTG
CCAATTTCCCTTCTTTCTCCCCAGCATATAAAGTCTCCATCTCTGGAACCACAGTAATATTGACA
TGCCCTGATATCTCGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGACCCCC
GCCCATTGACGTCAATAATGACGTATGTTCCCATAGTAACGCCAATAGGGACTTTCCATTGACGT
CAATGGGTGGAGTATTTACGGTAAACTGCCCACTTGGCAGTACATCAAGTGTATCATATGCCAAG
TACGCCCCCTATTGACGTCAATGACGGTAAATGGCCCGCCTGGCATTATGCCCAGTACATGACCT
TATGGGACTTTCCTACTTGGCAGTACATCTACGTATTAGTCATCGCTATTACCATGGTCGAGGTG
AGCCCCACGTTCTGCTTCACTCTCCCCATCTCCCCCCCCTCCCCACCCCCAATTTTGTATTTATT
TATTTTTTAATTATTTTGTGCAGCGATGGGGGCGGGGGGGGGGGGGGGGCGCGCGCCAGGCGGGG
CGGGGCGGGGCGAGGGGCGGGGCGGGGCGAGGCGGAGAGGTGCGGCGGCAGCCAATCAGAGCGGC
GCGCTCCGAAAGTTTCCTTTTATGGCGAGGCGGCGGCGGCGGCGGCCCTATAAAAAGCGAAGCGC
GCGGCGGGCGGGGAGTCGCTGCGACGCTGCCTTCGCCCCGTGCCCCGCTCCGCCGCCGCCTCGCG
CCGCCCGCCCCGGCTCTGACTGACCGCGTTACTCCCACAGGTGAGCGGGCGGGACGGCCCTTCTC
CTCCGGGCTGTAATTAGCGCTTGGTTTAATGACGGCTTGTTTCTTTTCTGTGGCTGCGTGAAAGC
CTTGAGGGGCTCCGGGAGGGCCCTTTGTGCGGGGGGAGCGGCTCGGGGGGTGCGTGCGTGTGTGT
GTGCGTGGGGAGCGCCGCGTGCGGCTCCGCGCTGCCCGGCGGCTGTGAGCGCTGCGGGCGCGGCG
CGGGGCTTTGTGCGCTCCGCAGTGTGCGCGAGGGGAGCGCGGCCGGGGGCGGTGCCCCGCGGTGC
GGGGGGGGCTGCGAGGGGAACAAAGGCTGCGTGCGGGGTGTGTGCGTGGGGGGGTGAGCAGGGGG
TGTGGGCGCGTCGGTCGGGCTGCAACCCCCCCTGCACCCCCCTCCCCGAGTTGCTGAGCACGGCC
CGGCTTCGGGTGCGGGGCTCCGTACGGGGCGTGGCGCGGGGCTCGCCGTGCCGGGCGGGGGGTGG
CGGCAGGTGGGGGTGCCGGGCGGGGCGGGGCCGCCTCGGGCCGGGGAGGGCTCGGGGGAGGGGCG
CGGCGGCCCCCGGAGCGCCGGCGGCTGTCGAGGCGCGGCGAGCCGCAGCCATTGCCTTTTATGGT
AATCGTGCGAGAGGGCGCAGGGACTTCCTTTGTCCCAAATCTGTGCGGAGCCGAAATCTGGGAGG
CGCCGCCGCACCCCCTCTAGCGGGCGCGGGGCGAAGCGGTGCGGCGCCGGCAGGAAGGAAATGGG
CGGGGAGGGCCTTCGTGCGTCGCCGCGCCGCCGTCCCCTTCTCCCTCTCCAGCCTCGGGGCTGTC
CGCGGGGGGACGGCTGCCTTCGGGGGGGACGGGGCAGGGCGGGGTTCGGCTTCTGGCGTGTGACC
GGCGGCTCTAGAGCCTCTGCTAACCATGTTCATGCCTTCTTCTTTTTCCTACAGCTCCTGGGCAA
CGTGCTGGTTATTGTGCTGTCTCATCATTTTGGCAAAGAATTAATTCGGATCCACCATGGCTCTC
CCAGTGACTGCCCTACTGCTTCCCCTAGCGCTTCTCCTGCATGCAGAGGTGAAGCTGCAGCAGTC
TGGGGCTGAGCTGGTGAGGCCTGGGTCCTCAGTGAAGATTTCCTGCAAGGCTTCTGGCTATGCAT
TCAGTAGCTACTGGATGAACTGGGTGAAGCAGAGGCCTGGACAGGGTCTTGAGTGGATTGGACAG
ATTTATCCTGGAGATGGTGATACTAACTACAATGGAAAGTTCAAGGGTCAAGCCACACTGACTGC
AGACAAATCCTCCAGCACAGCCTACATGCAGCTCAGCGGCCTAACATCTGAGGACTCTGCGGTCT
ATTTCTGTGCAAGAAAGACCATTAGTTCGGTAGTAGATTTCTACTTTGACTACTGGGGCCAAGGG
ACCACGGTCACCGTCTCCTCAGGTGGAGGTGGATCAGGaGGtGGaGGtTCTGGTGGAGGaGGATC
TGACATTGAGCTCACCCAGTCTCCAAAATTCATGTCCACATCAGTAGGAGACAGGGTCAGCGTCA
CCTGCAAGGCCAGTCAGAATGTGGGTACTAATGTAGCCTGGTATCAACAGAAACCAGGACAATCT
CCTAAACCACTGATTTACTCGGCAACCTACCGGAACAGTGGAGTCCCTGATCGCTTCACAGGCAG
TGGATCTGGGACAGATTTCACTCTCACCATCACTAACGTGCAGTCTAAAGACTTGGCAGACTATT
TCTGTCAACAATATAACAGGTATCCGTACACGTCCGGAGGGGGGACCAAGCTGGAGATCAAACGG
GCGGCCGCAATTGAAGTTATGTATCCTCCTACTTACCTAGACAATGAGAAGAGCAATGGAACCAT
TATCCATGTGAAAGGGAAACACCTTTGTCCAAGTCCCCTATTTCCCGGACCTTCTAAGCCCTTTT
GGGTGCTGGTGGTGGTTGGTGGAGTCCTGGCTTGCTATAGCTTGCTAGTAACAGTGGCCTTTATT
ATTTTCTGGGTGAGGAGTAAGAGGAGCAGGCTCCTGCACAGTGACTACATGAACATGACTCCtCG
CCGCCCCGGGCCtACaCGCAAGCATTACCAGCCCTATGCCCCACCACGCGACTTCGCAGCCTATC
GCTCCAGAGTGAAGTTCAGCAGGAGCGCAGAGCCCCCCGCGTACCAGCAGGGCCAGAACCAGCTC
TATAACGAGCTCAATCTAGGACGAAGAGAGGAGTACGATGTTTTGGACAAGAGACGTGGCCGGGA
CCCTGAGATGGGGGGAAAGCCGAGAAGGAAGAACCCTCAGGAAGGCCTGTACAATGAACTGCAGA
AAGATAAGATGGCGGAGGCCTACAGTGAGATTGGGATGAAAGGCGAGCGCCGGAGGGGCAAGGGG
CACGATGGCCTTTACCAGGGTCTCAGTACAGCCACCAAGGACACCTACGACGCCCTTCACATGCA
GGCCCTGCCCCCTCGCTAACGACTGTGCCTTCTAGTTGCCAGCCATCTGTTGTTTGCCCCTCCCC
CGTGCCTTCCTTGACCCTGGAAGGTGCCACTCCCACTGTCCTTTCCTAATAAAATGAGGAAATTG
CATCGCATTGTCTGAGTAGGTGTCATTCTATTCTGGGGGGTGGGGTGGGGCAGGACAGCAAGGGG
GAGGATTGGGAAGACAATAGCAGGCATGCTGGGGATGCGGTGGGCTCTATGGCAGTATCCTGGAT
CTGAAATACTATGGCAACACAatgataaaaacataggcggtgatgaggatgataaaaacataggc
agtgatgaggatCACCTGTCACTGAAGGAATTTTCAGAATTGGAGCAAAGTGGTTATTATGTCTG
CTACCCCAGAGGAAGCAAACCAGAAGATGCGAACTTTTATCTCTACCTGAGGGCAAGAGGTAATC
CAGGTCTCCAGAACAGGTACCACCGGCTCTTTAGGGAGGACCATTCAAAAGGGCATTCTCAGTGA
TTTTCCCTAACCCAGCTCACAGTGCCCAGGCGTCTTTGCGCTTCCTCCCACACTCAATCCTGGGA
CTCTCTGGTACCACACGGCATCAGTGTTTTCTGGAATATAGATTAAACACCAATATGAGGCTTCT
GGGTAACCCCAGTCTGTGCGAGATCTAAAATAGCAACTCCCTAAGAGACAGGACTGGGTCATTTG
CACCGCATCACACCCAGGTTCATAGCACACCAGCGGCCGCTTTCAGATCCAGGATACTGAGGGCA
TGTT
CD3E_34 CAG miniplasmid sequence:
(SEQ ID NO: 2051)
TTTCCATAGGCTCCGCCaCCCTGACGAGCA
TCACAAAAATCGACGCTCAAGTCAGAGGTGGCGAAACCCGACAGGACTATAAAGATACCAGGCGT
TTCCCCCTGGAAGCTCCCTCGTGCGCTCTCCTGTTCCGACCCTGCCGCTTACCGGATACCTGTCC
GCCTTTCTCCCTTCGGGAAGCGTGGCGCTTTCTCATAGCTCACGCTGTAGGTATCTCAGTTCGGT
GTAGGTCGTTCGCTCCAAGCTGGGCTGTGTGCACGAACCCCCCGTTCAGCCCGACCGCTGCGCCT
TATCCGGTAACTATCGTCTTGAGTCCAACCCGGTAAGACACGACTTATCGCCACTGGCAGCAGCC
ACTGGTAACAGGATTAGCAGAGCGAGGTATGTAGGCGGTGCTACAGAGTTCTTGAAGTGGTGGCC
TAACTACGGCTACACTAGAAGGACAGTATTTGGTATCTGCGCTCTGCTGAAGCCAGTTACCTTCG
GAAAAAGAGTTGGTAGCTCTTGATCCGGCAAACAAACCACCGCTGGTAGCGGTGGTTTTTTTGTT
TGCAAGCAGCAGATTACGCGCAGAAAAAAAGGATCTCAAGAAGATCCTTTGATCTTTTCTACGTC
AGTCCTGCTCCTCGGCCACGAAGTGCACGCAGTTGCCGGCCGGGTCGCGCAGGGCGAACTCCCGC
CCCCACGGCTGCTCGCCGATCTCGGTCATGGCCGGCCCGGAGGCGTCCCGGAAGTTCGTGGACAC
GACCTCCGACCACTCGGCGTACAGCTCGTCCAGGCCGCGCACCCACACCCAGGCCAGGGTGTTGT
CCGGCACCACCTGGTCCTGGACCGCGCTGATGAACAGGGTCACGTCGTCCCGGACCACACCGGCG
AAGTCGTCCTCCACGAAGTCCCGGGAGAACCCGAGCCGGTCGGTCCAGAACTCGACCGCTCCGGC
GACGTCGCGCGCGGTGAGCACCGGAACGGCACTGGTCAACTTGGCCATACTCTTCCTTTTCAATA
TTATTGAAGCATTTATCAGGGTTATTGTCTCATGAGCGGATACATAACGCGTGCCCTCAGTATCC
TGGATCTGAAAATTGGGATCCTCAGCAGACACGTGAGTTTATTGGTCTTTTATTTATGCCCTGTC
TGAGGATGCAGATTGGTGGGTAGATGAGAAGGAACTGATTGAGAGAGATTAACCCCAAGAACTGA
TATCTTCCCAGCATTGCATTCTCAACTCCATTTTAGAAAGGTTCCAAATAGGGACTTCTGTGGGT
TTTTCTTTACATCCATCTTACCCTTCCCAAGTCCCCATGTCCCTGCGTAAACCCTAAAGCCACCT
CTCAAaaggttctctagttcccttcaaggttctctagttcccttcaTTCCACATATCTCCTCTTC
CACACCCTCTAGCCAGTAGAGCTCCCTTCTGACAAGCAAGTCTAAGATCTAGATGACAGATGACT
TCCTGCATTTGGGTGGTTCTTTTGTCACTAATTTGCCTTTTCTAAAATTGTCCTGGTTTCTTCTG
CCAATTTCCCTTCTTTCTCCCCAGCATATAAAGTCTCCATCTCTGGAACCACAGTAATATTGACA
TGCCCTGATATCTCGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGACCCCC
GCCCATTGACGTCAATAATGACGTATGTTCCCATAGTAACGCCAATAGGGACTTTCCATTGACGT
CAATGGGTGGAGTATTTACGGTAAACTGCCCACTTGGCAGTACATCAAGTGTATCATATGCCAAG
TACGCCCCCTATTGACGTCAATGACGGTAAATGGCCCGCCTGGCATTATGCCCAGTACATGACCT
TATGGGACTTTCCTACTTGGCAGTACATCTACGTATTAGTCATCGCTATTACCATGGTCGAGGTG
AGCCCCACGTTCTGCTTCACTCTCCCCATCTCCCCCCCCTCCCCACCCCCAATTTTGTATTTATT
TATTTTTTAATTATTTTGTGCAGCGATGGGGGCGGGGGGGGGGGGGGGGCGCGCGCCAGGCGGGG
CGGGGCGGGGCGAGGGGCGGGGCGGGGCGAGGCGGAGAGGTGCGGCGGCAGCCAATCAGAGCGGC
GCGCTCCGAAAGTTTCCTTTTATGGCGAGGCGGCGGCGGCGGCGGCCCTATAAAAAGCGAAGCGC
GCGGCGGGCGGGGAGTCGCTGCGACGCTGCCTTCGCCCCGTGCCCCGCTCCGCCGCCGCCTCGCG
CCGCCCGCCCCGGCTCTGACTGACCGCGTTACTCCCACAGGTGAGCGGGCGGGACGGCCCTTCTC
CTCCGGGCTGTAATTAGCGCTTGGTTTAATGACGGCTTGTTTCTTTTCTGTGGCTGCGTGAAAGC
CTTGAGGGGCTCCGGGAGGGCCCTTTGTGCGGGGGGAGCGGCTCGGGGGGTGCGTGCGTGTGTGT
GTGCGTGGGGAGCGCCGCGTGCGGCTCCGCGCTGCCCGGCGGCTGTGAGCGCTGCGGGCGCGGCG
CGGGGCTTTGTGCGCTCCGCAGTGTGCGCGAGGGGAGCGCGGCCGGGGGCGGTGCCCCGCGGTGC
GGGGGGGGCTGCGAGGGGAACAAAGGCTGCGTGCGGGGTGTGTGCGTGGGGGGGTGAGCAGGGGG
TGTGGGCGCGTCGGTCGGGCTGCAACCCCCCCTGCACCCCCCTCCCCGAGTTGCTGAGCACGGCC
CGGCTTCGGGTGCGGGGCTCCGTACGGGGCGTGGCGCGGGGCTCGCCGTGCCGGGCGGGGGGTGG
CGGCAGGTGGGGGTGCCGGGCGGGGCGGGGCCGCCTCGGGCCGGGGAGGGCTCGGGGGAGGGGCG
CGGCGGCCCCCGGAGCGCCGGCGGCTGTCGAGGCGCGGCGAGCCGCAGCCATTGCCTTTTATGGT
AATCGTGCGAGAGGGCGCAGGGACTTCCTTTGTCCCAAATCTGTGCGGAGCCGAAATCTGGGAGG
CGCCGCCGCACCCCCTCTAGCGGGCGCGGGGCGAAGCGGTGCGGCGCCGGCAGGAAGGAAATGGG
CGGGGAGGGCCTTCGTGCGTCGCCGCGCCGCCGTCCCCTTCTCCCTCTCCAGCCTCGGGGCTGTC
CGCGGGGGGACGGCTGCCTTCGGGGGGGACGGGGCAGGGCGGGGTTCGGCTTCTGGCGTGTGACC
GGCGGCTCTAGAGCCTCTGCTAACCATGTTCATGCCTTCTTCTTTTTCCTACAGCTCCTGGGCAA
CGTGCTGGTTATTGTGCTGTCTCATCATTTTGGCAAAGAATTAATTCGGATCCACCATGGCTCTC
CCAGTGACTGCCCTACTGCTTCCCCTAGCGCTTCTCCTGCATGCAGAGGTGAAGCTGCAGCAGTC
TGGGGCTGAGCTGGTGAGGCCTGGGTCCTCAGTGAAGATTTCCTGCAAGGCTTCTGGCTATGCAT
TCAGTAGCTACTGGATGAACTGGGTGAAGCAGAGGCCTGGACAGGGTCTTGAGTGGATTGGACAG
ATTTATCCTGGAGATGGTGATACTAACTACAATGGAAAGTTCAAGGGTCAAGCCACACTGACTGC
AGACAAATCCTCCAGCACAGCCTACATGCAGCTCAGCGGCCTAACATCTGAGGACTCTGCGGTCT
ATTTCTGTGCAAGAAAGACCATTAGTTCGGTAGTAGATTTCTACTTTGACTACTGGGGCCAAGGG
ACCACGGTCACCGTCTCCTCAGGTGGAGGTGGATCAGGaGGtGGaGGtTCTGGTGGAGGaGGATC
TGACATTGAGCTCACCCAGTCTCCAAAATTCATGTCCACATCAGTAGGAGACAGGGTCAGCGTCA
CCTGCAAGGCCAGTCAGAATGTGGGTACTAATGTAGCCTGGTATCAACAGAAACCAGGACAATCT
CCTAAACCACTGATTTACTCGGCAACCTACCGGAACAGTGGAGTCCCTGATCGCTTCACAGGCAG
TGGATCTGGGACAGATTTCACTCTCACCATCACTAACGTGCAGTCTAAAGACTTGGCAGACTATT
TCTGTCAACAATATAACAGGTATCCGTACACGTCCGGAGGGGGGACCAAGCTGGAGATCAAACGG
GCGGCCGCAATTGAAGTTATGTATCCTCCTACTTACCTAGACAATGAGAAGAGCAATGGAACCAT
TATCCATGTGAAAGGGAAACACCTTTGTCCAAGTCCCCTATTTCCCGGACCTTCTAAGCCCTTTT
GGGTGCTGGTGGTGGTTGGTGGAGTCCTGGCTTGCTATAGCTTGCTAGTAACAGTGGCCTTTATT
ATTTTCTGGGTGAGGAGTAAGAGGAGCAGGCTCCTGCACAGTGACTACATGAACATGACTCCtCG
CCGCCCCGGGCCtACaCGCAAGCATTACCAGCCCTATGCCCCACCACGCGACTTCGCAGCCTATC
GCTCCAGAGTGAAGTTCAGCAGGAGCGCAGAGCCCCCCGCGTACCAGCAGGGCCAGAACCAGCTC
TATAACGAGCTCAATCTAGGACGAAGAGAGGAGTACGATGTTTTGGACAAGAGACGTGGCCGGGA
CCCTGAGATGGGGGGAAAGCCGAGAAGGAAGAACCCTCAGGAAGGCCTGTACAATGAACTGCAGA
AAGATAAGATGGCGGAGGCCTACAGTGAGATTGGGATGAAAGGCGAGCGCCGGAGGGGCAAGGGG
CACGATGGCCTTTACCAGGGTCTCAGTACAGCCACCAAGGACACCTACGACGCCCTTCACATGCA
GGCCCTGCCCCCTCGCTAACGACTGTGCCTTCTAGTTGCCAGCCATCTGTTGTTTGCCCCTCCCC
CGTGCCTTCCTTGACCCTGGAAGGTGCCACTCCCACTGTCCTTTCCTAATAAAATGAGGAAATTG
CATCGCATTGTCTGAGTAGGTGTCATTCTATTCTGGGGGGTGGGGTGGGGCAGGACAGCAAGGGG
GAGGATTGGGAAGACAATAGCAGGCATGCTGGGGATGCGGTGGGCTCTATGGCAGTATCCTGGAT
CTGAAATACTATGGCAACACAatgataaaaacataggcggtgatgaggatgataaaaacataggc
agtgatgaggatCACCTGTCACTGAAGGAATTTTCAGAATTGGAGCAAAGTGGTTATTATGTCTG
CTACCCCAGAGGAAGCAAACCAGAAGATGCGAACTTTTATCTCTACCTGAGGGCAAGAGGTAATC
CAGGTCTCCAGAACAGGTACCACCGGCTCTTTAGGGAGGACCATTCAAAAGGGCATTCTCAGTGA
TTTTCCCTAACCCAGCTCACAGTGCCCAGGCGTCTTTGCGCTTCCTCCCACACTCAATCCTGGGA
CTCTCTGGTACCACACGGCATCAGTGTTTTCTGGAATATAGATTAAACACCAATATGAGGCTTCT
GGGTAACCCCAGTCTGTGCGAGATCTAAAATAGCAACTCCCTAAGAGACAGGACTGGGTCATTTG
CACCGCATCACACCCAGGTTCATAGCACACCAGCGGCCGCTTTCAGATCCAGGATACTGAGGGCA
TGTT
PLA074-TRAC043 P2A miniplasmid sequence:
(SEQ ID NO: 2052)
AGGCTAGGTGGAGGCTCAGTGATG
ATAAGTCTGCGATGGTGGATGCATGTGTCATGGTCATAGCTGTTTCCTGTGTGAAATTGTTATCC
GCTCAGAGGGCACAATCCTATTCCGCGCTATCCGACAATCTCCAAGACATTAGGTGGAGTTCAGT
TCGGCGTATGGCATATGTCGCTGGAAAGAACATGTGAGCAAAAGGCCAGCAAAAGGCCAGGAACC
GTAAAAAGGCCGCGTTGCTGGCGTTTTTCCATAGGCTCCGCCCCCCTGACGAGCATCACAAAAAT
CGACGCTCAAGTCAGAGGTGGCGAAACCCGACAGGACTATAAAGATACCAGGCGTTTCCCCCTGG
AAGCTCCCTCGTGCGCTCTCCTGTTCCGACCCTGCCGCTTACCGGATACCTGTCCGCCTTTCTCC
CTTCGGGAAGCGTGGCGCTTTCTCATAGCTCACGCTGTAGGTATCTCAGTTCGGTGTAGGTCGTT
CGCTCCAAGCTGGGCTGTGTGCACGAACCCCCCGTTCAGCCCGACCGCTGCGCCTTATCCGGTAA
CTATCGTCTTGAGTCCAACCCGGTAAGACACGACTTATCGCCACTGGCAGCAGCCACTGGTAACA
GGATTAGCAGAGCGAGGTATGTAGGCGGTGCTACAGAGTTCTTGAAGTGGTGGCCTAACTACGGC
TACACTAGAAGAACAGTATTTGGTATCTGCGCTCTGCTGAAGCCAGTTACCTTCGGAAAAAGAGT
TGGTAGCTCTTGATCCGGCAAACAAACCACCGCTGGTAGCGGTGGTTTTTTTGTTTGCAAGCAGC
AGATTACGCGCAGAAAAAAAGGATCTCAAGAAGATCCTTTGATCTTTTCTACGGGGTCTGACGCT
CTATTCAACAAAGCCGCCGTCCCGTCAAGTCAGCGTAAATGGGTAGGGGGCTTCAAATCGTCCTC
GTGATACCAATTCGGAGCCTGCTTTTTTGTACAAACTTGTTGATAATGGCAATTCAAGGATCTTC
ACCTAGATCCTTTTAAATTAAAAATGAAGTTTTAAATCAATCTAAAGTATATATGAGTAAACTTG
GTCTGACAGTTACCAATGCTTAATCAGTGAGGCACCTATCTCAGCGATCTGTCTATTTCGTTCAT
CCATAGTTGCCTGACTCCCCGTCGTGTAGATAACTACGATACGGGAGGGCTTACCATCTGGCCCC
AGTGCTGCAATGATACCGCGAGAGCCACGCTCACCGGCTCCAGATTTATCAGCAATAAACCAGCC
AGCCGGAAGGGCCGAGCGCAGAAGTGGTCCTGCAACTTTATCCGCCTCCATCCAGTCTATTAATT
GTTGCCGGGAAGCTAGAGTAAGTAGTTCGCCAGTTAATAGTTTGCGCAACGTTGTTGCCATTGCT
ACAGGCATCGTGGTGTCACGCTCGTCGTTTGGTATGGCTTCATTCAGCTCCGGTTCCCAACGATC
AAGGCGAGTTACATGATCCCCCATGTTGTGCAAAAAAGCGGTTAGCTCCTTCGGTCCTCCGATCG
TTGTCAGAAGTAAGTTGGCCGCAGTGTTATCACTCATGGTTATGGCAGCACTGCATAATTCTCTT
ACTGTCATGCCATCCGTAAGATGCTTTTCTGTGACTGGTGAGTACTCAACCAAGTCATTCTGAGA
ATAGTGTATGCGGCGACCGAGTTGCTCTTGCCCGGCGTCAATACGGGATAATACCGCGCCACATA
GCAGAACTTTAAAAGTGCTCATCATTGGAAAACGTTCTTCGGGGCGAAAACTCTCAAGGATCTTA
CCGCTGTTGAGATCCAGTTCGATGTAACCCACTCGTGCACCCAACTGATCTTCAGCATCTTTTAC
TTTCACCAGCGTTTCTGGGTGAGCAAAAACAGGAAGGCAAAATGCCGCAAAAAAGGGAATAAGGG
CGACACGGAAATGTTGAATACTCATACTCTTCCTTTTTCAATATTATTGAAGCATTTATCAGGGT
TATTGTCTCATGAGCGGATACATATTTGAATGTATTTAGAAAAATAAACAAATAGGGGTTCCGCG
CACATTTCCCCGAAAAGTGCCAGATACCTGAAACAAAACCCATCGTACGGCCAAGGAAGTCTCCA
ATAACTGTGATCCACCACAAGCGCCAGGGTTTTCCCAGTCACGACGTTGTAAAACGACGGCCAGT
CATGCATAATCCGCACGCATCTGGAATAAGGAAGTGCCATTCCGCCTGACCTCCTCAGCAATGCC
AACATACCATAAACCTCCCATTCTGCTAATGCCCAGCCTAAGTTGGGGAGACCACTCCAGATTCC
AAGATGTACAGTTTGCTTTGCTGGGCCTTTTTCCCATGCCTGCCTTTACTCTGCCAGAGTTATAT
TGCTGGGGTTTTGAAGAAGATCCTATTAAATAAAAGAATAAGCAGTATTATTAAGTAGCCCTGCA
TTTCAGGTTTCCTTGAGTGGCAGGCCAGGCCTGGCCGTGAACGTTCACTGAAATCATGGCCTCTT
GGCCAAGATTGATAGCTTGTGCCTGTCCCTGAGTCCCAGTCCATCACGAGCAGCTGGTTTCTAAG
ATGCTATTTCCCGTATAAAGCATGAGACCGTGACTTGCCAGCCCCACAGAGCCCCGCCCTTGTCC
ATCACTGGCATCTGGACTCCAGCCTGGGTTGGGGCAAAGAGGGAAATGAGATCATGTCCTAACCC
TGATCCTCTTGTCCCACAGATATCCAGAACCCTGACCCTGCCGTGGGCAGCGGCGCTACTAACTT
CAGCCTGCTGAAGCAGGCTGGCGACGTGGAGGAGAACCCTGGACCTATGGCTCTCCCAGTGACTG
CCCTACTGCTTCCCCTAGCGCTTCTCCTGCATGCAGAGGTGAAGCTGCAGCAGTCTGGGGCTGAG
CTGGTGAGGCCTGGGTCCTCAGTGAAGATTTCCTGCAAGGCTTCTGGCTATGCATTCAGTAGCTA
CTGGATGAACTGGGTGAAGCAGAGGCCTGGACAGGGTCTTGAGTGGATTGGACAGATTTATCCTG
GAGATGGTGATACTAACTACAATGGAAAGTTCAAGGGTCAAGCCACACTGACTGCAGACAAATCC
TCCAGCACAGCCTACATGCAGCTCAGCGGCCTAACATCTGAGGACTCTGCGGTCTATTTCTGTGC
AAGAAAGACCATTAGTTCGGTAGTAGATTTCTACTTTGACTACTGGGGCCAAGGGACCACGGTCA
CCGTCTCCTCAGGTGGAGGTGGATCAGGTGGAGGTGGATCTGGTGGAGGTGGATCTGACATTGAG
CTCACCCAGTCTCCAAAATTCATGTCCACATCAGTAGGAGACAGGGTCAGCGTCACCTGCAAGGC
CAGTCAGAATGTGGGTACTAATGTAGCCTGGTATCAACAGAAACCAGGACAATCTCCTAAACCAC
TGATTTACTCGGCAACCTACCGGAACAGTGGAGTCCCTGATCGCTTCACAGGCAGTGGATCTGGG
ACAGATTTCACTCTCACCATCACTAACGTGCAGTCTAAAGACTTGGCAGACTATTTCTGTCAACA
ATATAACAGGTATCCGTACACGTCCGGAGGGGGGACCAAGCTGGAGATCAAACGGGCGGCCGCAA
TTGAAGTTATGTATCCTCCTACTTACCTAGACAATGAGAAGAGCAATGGAACCATTATCCATGTG
AAAGGGAAACACCTTTGTCCAAGTCCCCTATTTCCCGGACCTTCTAAGCCCTTTTGGGTGCTGGT
GGTGGTTGGTGGAGTCCTGGCTTGCTATAGCTTGCTAGTAACAGTGGCCTTTATTATTTTCTGGG
TGAGGAGTAAGAGGAGCAGGCTCCTGCACAGTGACTACATGAACATGACTCCCCGCCGCCCCGGG
CCCACCCGCAAGCATTACCAGCCCTATGCCCCACCACGCGACTTCGCAGCCTATCGCTCCAGAGT
GAAGTTCAGCAGGAGCGCAGAGCCCCCCGCGTACCAGCAGGGCCAGAACCAGCTCTATAACGAGC
TCAATCTAGGACGAAGAGAGGAGTACGATGTTTTGGACAAGAGACGTGGCCGGGACCCTGAGATG
GGGGGAAAGCCGAGAAGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCAAGGGGCACGATGGCC
TTTACCAGGGTCTCAGTACAGCCACCAAGGACACCTACGACGCCCTTCACATGCAGGCCCTGCCC
CCTCGCTAACGACTGTGCCTTCTAGTTGCCAGCCATCTGTTGTTTGCCCCTCCCCCGTGCCTTCC
TTGACCCTGGAAGGTGCCACTCCCACTGTCCTTTCCTAATAAAATGAGGAAATTGCATCGCATTG
TCTGAGTAGGTGTCATTCTATTCTGGGGGGTGGGGTGGGGCAGGACAGCAAGGGGGAGGATTGGG
AAGACAATAGCAGGCATGCTGGGGATACCAGCTGAGAGACTCTAATTCCAGTGACAAGTCTGTCT
GCCTATTCACCGATTTTGATTCTCAAACAAATGTGTCACAAAGTAAGGATTCTGATGTGTATATC
ACAGACAAAACTGTGCTAGACATGAGGTCTATGGACTTCAAGAGCAACAGTGCTGTGGCCTGGAG
CAACAAATCTGACTTTGCATGTGCAAACGCCTTCAACAACAGCATTATTCCAGAAGACACCTTCT
TCCCCAGCCCAGGTAAGGGCAGCTTTGGTGCCTTCGCAGGCTGTTTCCTTGCTTCAGGAATGGCC
AGGTTCTGCCCAGAGCTCTGGTCAATGATGTCTAAAACTCCTCTGATTGGTGGTCTCGGCCTTAT
CCATTGCCACCAAAACCCTCTTTTTACTAAGAAACAGTGAGCCTTGTTCTGGCAGTCCAGAGAAT
GACACGGGAAAAAAGCAGATGAAGAGAAGGTGGCAGGAGAGGGCACGTGGCCCAGCCTCAGTCTC
TCCAACTGAGTTCCTGCCTGCCTGCCTTTGCTCAGACTGTTTGCCCCTTACTGCTCTTCTAGGCC
TCATTCTAAGCCCCTTCTCCAAGTTGCCTCTCCTTATTTCTCCCTGTCTGCCAAGCGGCCGC

### IX. EQUIVALENTS

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The terms “a” and “an” and “the” and similar references in the context of describing the invention (especially in the context of the following claims) are to be construed to cover both the singular and the plural, unless otherwise indicated herein or clearly contradicted by context. For example, the term “a cell” includes a plurality of cells, including mixtures thereof. Where the plural form is used for compounds, salts, or the like, this is taken to mean also a single compound, salt, or the like.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a +10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A composition comprising a modified human cell comprising:
(a) a first genomic modification comprising a first portion of a first polynucleotide, wherein the first portion codes for a first chimeric antigen receptor (CAR) or portion thereof, inserted into a site with a TRAC gene, whereby the TRAC gene is partially or completely inactivated and the first CAR or portion thereof is expressed; and
(b) a second genomic modification comprising a second polynucleotide coding for a fusion protein of B2M and HLA-E or HLA-G inserted into a B2M gene, whereby endogenous B2M is partially or completely inactivated and the fusion protein is expressed.

2. The composition of claim 1, wherein the TRAC gene is completely inactivated.

3. The composition of claim 1 or claim 2, wherein the endogenous B2M gene is completely inactivated.

4. The composition of any one of claims 1-3, further comprising:
(c) a third genomic modification in a CIITA gene, wherein the CIITA gene is partially or completely inactivated.

5. The composition of claim 4, wherein the CIITA gene is completely inactivated.

6. The composition of claim 4 or claim 5, wherein the third genomic modification comprises a substitution, an insertion, a deletion, a nonsense mutation, or a truncation.

7. The composition of any one of claims 1 through 6, wherein the CAR or portion thereof comprises a polypeptide that binds at least one of B7H3, BCMA, GPRC5D, CD8, CD8a, CD19, CD20, CD22, CD28, 4-1BB, or CD3zeta.

8. The composition of claim 7, wherein the CAR or portion thereof comprises a the CAR or portion thereof comprises a polypeptide at least 60, at least 70, at least 80, at least 90, at least 95, at least 99%, or 100% identical to any one of the amino acid sequences of SEQ ID NOS: 86-124.

9. The composition of claim 1 or claim 6, wherein the CAR or portion thereof comprises a polypeptide that binds at least one of B7H3, BCMxA, GPRC5D, CD8, CD8a, CD20, CD22, CD28, 4-1BB, or CD3zeta.

10. The composition of claim 9, wherein the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

11. The composition of any one of claims 1 through 10, further comprising a second portion of the first polynucleotide, wherein the second portion codes for a second CAR or portion thereof, different from the first CAR or portion thereof.

12. A composition comprising a modified human cell comprising:
(a) a first genomic modification comprising a first portion of a polynucleotide, wherein the first portion codes for a first chimeric antigen receptor (CAR) or portion thereof, inserted into a site with a TRAC gene, whereby the TRAC gene is partially or completely inactivated and the first CAR or portion thereof is expressed; and
(b) a second genomic modification in a CIITA gene, wherein the CIITA gene is partially or completely inactivated.

13. The composition of claim 12, wherein the TRAC gene is completely inactivated.

14. The composition of claim 12 or claim 13, wherein the CIITA gene is completely inactivated.

15. The composition of any one of claims 12 through 14, further comprising:
(c) a third genomic modification comprising a polynucleotide coding for a fusion protein of B2M and HLA-E or HLA-G inserted into a B2M gene, whereby endogenous B2M is partially or completely inactivated and the fusion protein is expressed.

16. The composition of claim 15, wherein endogenous B2M is completely inactivated.

17. The composition of claim 12, wherein the second genomic modification comprises a substitution, an insertion, a deletion, a nonsense mutation, or a truncation.

18. The composition of any one of claims 12 through 17, wherein the CAR or portion thereof comprises a polypeptide that binds at least one of B7H3, BCMA, GPRC5D, CD8, CD8a, CD19, CD20, CD22, CD28, 4-1BB, or CD3zeta.

19. The composition of claim 18, wherein the CAR or portion thereof comprises a polypeptide at least 60, at least 70, at least 80, at least 90, at least 95, at least 99%, or 100% identical to any one of the amino acid sequences of SEQ ID NOs: 86-124.

20. The composition of any one of claims 12 through 17, wherein the CAR or portion thereof comprises a polypeptide that binds at least one of B7H3, BCMA, GPRC5D, CD8, CD8a, CD20, CD22, CD28, 4-1BB, or CD3zeta.

21. The composition of claim 20, wherein the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

22. The composition of any one of claims 12 through 21, further comprising a second portion of the polynucleotide, wherein the second potion codes for a second CAR or portion thereof, different from the first CAR or portion thereof.

23. A composition comprising a modified human cell comprising:
(a) a first genomic modification comprising a polynucleotide coding for a fusion protein of B2M and HLA-E or HLA-G inserted into a B2M gene, whereby endogenous B2M is partially or completely inactivated and the fusion protein is expressed; and
(b) a second genomic modification in a CIITA gene, wherein the CIITA gene is partially or completely inactivated.

24. The composition of claim 23, wherein the endogenous B2M gene is completely inactivated.

25. The composition of claim 23 or claim 24, wherein the CIITA gene is completely inactivated.

26. The composition of claim 25, wherein the second genomic modification comprises a substitution, an insertion, a deletion, a nonsense mutation, or a truncation.

27. The composition of any one of claims 23 through 26, further comprising:
(c) a third genomic modification comprising a first portion of a polynucleotide, wherein the first portion codes for a first chimeric antigen receptor (CAR) or portion thereof, inserted into a site with a TRAC gene, whereby the TRAC gene is partially or completely inactivated and the first CAR or portion thereof is expressed.

28. The composition of claim 27, wherein the CAR or portion thereof comprises a polypeptide that binds at least one of B7H3, BCMA, GPRC5D, CD8, CD8a, CD19, CD20, CD22, CD28, 4-1BB, or CD3zeta.

29. The composition of claim 28, wherein the CAR or portion thereof comprises a polypeptide at least 60, at least 70, at least 80, at least 90, at least 95, at least 99%, or 100% identical to any one of the amino acid sequences of SEQ ID NOs: 86-124.

30. The composition of claim 27, wherein the CAR or portion thereof comprises a polypeptide that binds at least one of B7H3, BCMxA, GPRC5D, CD8, CD8a, CD20, CD22, CD28, 4-1BB, or CD3zeta.

31. The composition of claim 29, wherein the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

32. The composition of any one of claims 27 through 31, further comprising a second portion of the first polynucleotide, wherein the second portion codes for a second CAR or portion thereof, different from the first CAR or portion thereof.

33. The composition of any one of claims 1 through 32, wherein the cell comprises an immune cell or a stem cell.

34. The composition of claim 33, wherein the cell comprises an immune cell comprising a neutrophil, eosinophil, basophil, mast cell, monocyte, macrophage, dendritic cell, natural killer cell, or a lymphocyte.

35. The composition of claim 33, wherein the cell comprises a T cell.

36. The composition of claim 33, wherein the cell comprises a stem cell comprising a human pluripotent, multipotent stem cell, embryonic stem cell, induced pluripotent stem cell, hematopoietic stem cell, or a CD34+ cell.

37. The composition of claim 33, wherein the cell comprises a stem cell comprising an iPSC.

38. The composition of any one of claims 1 through 37, further comprising a nuclease system or one or more polynucleotides encoding for one or more parts of the system comprising:
(1) a nucleic acid-guided nuclease; and
(2) a guide nucleic acid compatible with and capable of binding to and activating the nucleic acid-guided nuclease and comprising a spacer sequence complementary to a target nucleotide sequence in a polynucleotide of a human genome;
wherein, contacting the target polynucleotide with the nuclease system results in a strand break in at least one strand of the target polynucleotide of the genome of the human cell at or near the target nucleotide sequence.

39. The composition of claim 38, wherein the nucleic acid-guided nuclease comprises an engineered, non-naturally occurring nuclease.

40. The composition of claim 38 or claim 39, wherein the nucleic acid-guided nuclease comprises a Class 1 or a Class 2 nuclease.

41. The composition of claim 40, wherein the nucleic acid-guided nuclease comprises a Type II or a Type V nuclease.

42. The composition of claim 41, wherein the nucleic acid-guided nuclease comprises a Type V-A, V-B, V-C, V-D, or V-E nuclease.

43. The composition of claim 42, wherein the nucleic acid-guided nuclease comprises a Type V-A nuclease.

44. The composition of claim 43, wherein the nucleic acid-guided nuclease comprises a MAD nuclease, an ART nuclease, or an ABW nuclease.

45. The composition of claim 44, wherein the nucleic acid-guided nuclease comprises an amino acid sequence at least 80%, at least 85%, at least 90%, at least 95%, at least 99%, or 100% identical to an amino acid sequence of a MAD, ART, or ABW nuclease.

46. The composition of claim 44, wherein the nucleic acid-guided nuclease comprises a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20 nuclease.

47. The composition of claim 44, wherein the nucleic acid-guided nuclease comprises an ART1, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35 nuclease.

48. The composition of claim 44, wherein the nucleic acid-guided nuclease comprises an amino acid sequence at least 80%, at least 85%, at least 90%, at least 95%, at least 99%, or 100% identical, to the amino acid sequence of MAD2, MAD7, ART2, ART11, or ART11*.

49. The composition of claim 44, wherein the nucleic acid-guided nuclease comprises an amino acid sequence that is at least 80%, at least 85%, at least 90%, at least 95%, 99%, or 100% identical, to the amino acid sequence of SEQ ID NO: 37.

50. The composition of any one of claims 38 through 49, wherein the nucleic acid-guided nuclease further comprises at least one nuclear localization signal (NLS), at least one purification tag, and/or at least one cleavage site.

51. The composition of claim 50, wherein the nucleic acid-guided nuclease comprises at least 4 nuclear localization signals (NLS).

52. The composition of claim 51, wherein the nucleic acid-guided nuclease comprises one N-terminal and three C-terminal nuclease localization signals (NLS).

53. The composition of any one of claims 50 through 52, wherein the nuclear localization signals comprise any one of SEQ ID NOs: 40-56.

54. The composition of claim 32, wherein the NLS comprises SEQ ID NOs: 40, 51, and 56.

55. The composition of claim 38, wherein the guide nucleic acid comprises:
(i) a targeter nucleic acid comprising a targeter stem sequence and the spacer sequence; and
(ii) a modulator nucleic acid comprising a modulator stem sequence complementary to the targeter stem sequence, and, optionally, a 5′ sequence.

56. The composition of claim 55, wherein the guide nucleic acid comprises a single polynucleotide.

57. The composition of claim 55 or claim 56, wherein the guide nucleic acid comprises an engineered, non-naturally occurring guide nucleic acid.

58. The composition of claim 55 or claim 57, wherein the guide nucleic acid comprises a dual guide nucleic acid, wherein the targeter nucleic acid and the modulator nucleic acid are separate polynucleotides.

59. The composition of claim 58, wherein the dual guide nucleic acid is capable of binding to and activating a nucleic acid-guided nuclease, that, in a naturally occurring system, is activated by a single crRNA in the absence of a tracrRNA.

60. The composition of any one of claims 38 through 59, wherein the target nucleotide sequence is within at least 10, 20, 30, 40, or 50 nucleotides of a protospacer adjacent motif (PAM) that is recognized by the nucleic acid-guided nuclease.

61. The composition of any one of claims 38 through 60, wherein the guide nucleic acid and the nucleic acid-guided nuclease form a nucleic acid-guided nuclease complex.

62. The composition of claim 61, wherein the guide nucleic acid further comprises a donor template recruiting sequence.

63. The composition of claim 38 through 62, wherein the guide nucleic acid comprises a heterologous spacer sequence.

64. The composition of any one of claims 38 through 63, wherein the spacer sequence is at least 80%, at least 85%, at least 90%, at least 95%, at least 99.5%, or 100% identical to any one of any one of SEQ ID NOs: 125-2019.

65. The composition of any one of claims 38 through 64, wherein some or all of the guide nucleic acid comprises RNA.

66. The composition of claim 65, wherein at least 50%, at least 70%, at least 90%, at least 95%, or 100% of the guide nucleic acid comprises RNA.

67. The composition of any one of claims 38 through 66, wherein the guide nucleic acid comprises one or more chemical modifications to one or more nucleotides and/or internucleotide linkages at or near the 5′ end, at or near the 3′ end, and/or both.

68. The composition of claim 67, wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, or a combination thereof.

69. The composition of any one of claims 38 through 68, further comprising one or more donor templates.

70. The composition of claim 69, wherein the donor template comprises single-stranded DNA, linear single-stranded RNA, linear double-stranded DNA, linear double-stranded RNA, circular single-stranded DNA, circular single-stranded RNA, circular double-stranded DNA, or circular double-stranded RNA.

71. The composition of claim 69 or claim 70, wherein the donor template comprises two homology arms.

72. The composition of claim 71, wherein the homology arms comprise at least 50, 100, 150, 200, 250, 300, 350, 400, 450, 500, 600, 700, 800, or 900 and/or at most 100, 150, 200, 250, 300, 350, 400, 450, 500, 600, 700, 800, 900, or 1000 nucleotides, for example 50-1000 nucleotides, preferably 100-800 nucleotides, more preferably 250-750 nucleotides, even more preferably 400-600 nucleotides.

73. The composition of any one of claims claim 69 through 72, wherein the donor template comprises a mutation in a PAM sequence to partially or completely abolish binding of the RNP to the DNA.

74. The composition of any one of claims 69 through 73, wherein the donor template comprises one or more promoters.

75. The composition of claim 74, wherein the promoter shares at least 70%, at least 75%, at least 80%, at least 85%, at least 90%, at least 95%, at least 99.5%, or 100% sequence identity with any one of SEQ ID NOs: 78-85.

76. The composition of any one of claims 69 through 75, wherein the donor template comprises one or more chemical modifications to one or more nucleotides and/or internucleotide linkages at or near the 5′ end, at or near the 3′ end, or both.

77. The composition of claim 76, wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, a suitable alternative, or a combination thereof.

78. The composition of any one of claims 69 through 77, wherein the at least portion of the donor template is inserted by an innate cell repair mechanism.

79. The composition of claim 78, wherein the innate cell repair mechanism comprises homology directed repair (HDR).

80. A composition comprising a plurality of cell populations comprising:
(a) a first cell population comprising a plurality of the modified human cells of any one of claims 1 through 11; and
(b) a second cell population comprising a plurality of modified human cells wherein the second cell population does not comprise a modified human cell of the first population.

81. The composition of claim 80, wherein the first population of cells comprises at least 1, 2, 3, 4, 5, 6, 7, 8, 9, 10, 15, 20, 25, 30, 35, 40, 45, 50, 55, 60, 65, or 70% and/or not more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations, preferably 1-10%, more preferably 1-20%, even more preferably 1-30%, yet even more preferably 1-40%.

82. The composition of claim 80 or claim 81, wherein the second population of cells comprises at least 1, 2, 3, 4, 5, 6, 7, 8, 9, 10, 15, 20, 25, 30, 35, 40, 45, 50, 55, 60, 65, or 70% and/or no more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations, preferably 1-10%, more preferably 1-20%, even more preferably 1-30%, yet even more preferably 1-40%.

83. The composition of any one of claims 80 through 82, further comprising a third cell population wherein the third cell population does not contain a modified human cell of either the first or the second cell population.

84. The composition of claim 83, wherein the third population of cells comprises at least 1, 2, 3, 4, 5, 6, 7, 8, 9, 10, 15, 20, 25, 30, 35, 40, 45, 50, 55, 60, 65, or 70% and/or no more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations, preferably 1-10%, more preferably 1-20%, even more preferably 1-30%, yet even more preferably 1-40%.

85. The composition of any one of claims 80 through 84, further comprising a fourth cell population wherein the fourth cell population does not contain a modified human cell of either the first, second, or third cell population.

86. The composition of claim 85, wherein the fourth population of cells comprises at least 1, 2, 3, 4, 5, 6, 7, 8, 9, 10, 15, 20, 25, 30, 35, 40, 45, 50, 55, 60, 65, or 70% and/or no more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations, preferably 1-10%, more preferably 1-20%, even more preferably 1-30%, yet even more preferably 1-40%.

87. A composition comprising a plurality of cell populations comprising:
(a) a first cell population comprising a plurality of the modified human cells of any one of claims 4 through 11; and
(b) a second cell population comprising a plurality of modified human cells wherein the second cell population does not comprise a modified human cell of any one of claims 4 through 11.

88. The composition of claim 87 further comprising a third cell population wherein the third cell population does not contain a modified human cell of claim 4 through 11 or a modified human cell of the second cell population.

89. The composition of any one of claims 80 through 88, further comprising a pharmaceutically acceptable excipient.

90. A composition comprising a plurality of cell populations comprising:
(a) a first cell population comprising a plurality of cells wherein each cell comprises:
(i) a first genomic modification whereby a first gene that codes for a subunit of a TCR is partially or completely inactivated;
(ii) a second genomic modification whereby a second gene that codes for a subunit of an HLA-1 protein is partially or completely inactivated;
(iii) a third genomic modification whereby a third gene that codes for a subunit of an HLA-2 protein or that codes for a transcription factor for one or more subunits of an HLA-2 protein is partially or completely inactivated; and
(b) a second cell population, different from the first, wherein the second cell population comprises a plurality of cells that do not comprise one or more of genomic modifications of (i) through (iii), wherein each cell of the second population comprises the same genomic modifications.

91. The composition of claim 90, wherein the first cell population comprises at least 1, 2, 3, 4, 5, 6, 7, 8, 9, 10, 15, 20, 25, 30, 35, 40, 45, 50, 55, 60, 65, or 70% and/or no more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations, preferably 1-10%, more preferably 1-20%, even more preferably 1-30%, yet even more preferably 1-40%.

92. The composition of claim 90 or claim 91, wherein the second cell population comprises at least 1, 2, 3, 4, 5, 6, 7, 8, 9, 10, 15, 20, 25, 30, 35, 40, 45, 50, 55, 60, 65, or 70% and/or no more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations, preferably 1-10%, more preferably 1-20%, even more preferably 1-30%, yet even more preferably 1-40%.

93. The composition of any one of claims 90 through 92, wherein the first cell population further comprises:
(iv) a fourth genomic modification comprising a first portion of a polynucleotide, wherein the first portion codes for a first chimeric antigen receptor (CAR) or portion thereof, inserted into the first gene coding for a subunit of the T cell receptor (TCR) or into a safe harbor site, whereby the first CAR or portion thereof is expressed.

94. The composition of claim 93, wherein the subunit of a TCR protein comprises an alpha subunit or a beta subunit or a TRAC, TRBC, CD3E, CD3D, CD3G, or CD3Z gene.

95. The composition of claim 94, wherein the subunit of a TCR protein is an alpha 95. subunit.

96. The composition of claim 95, wherein the gene coding for the subunit of a TCR protein is a TRAC gene.

97. The composition of claim 90 or claim 96, wherein the first cell population further comprises:
(v) a fifth genomic modification comprising a polynucleotide coding for a fusion protein of B2M and a subunit of an HLA-1 protein inserted into a site within the second gene or a safe harbor site, whereby the fusion protein is expressed.

98. The composition of claim 97, wherein the first subunit comprises B2M.

99. The composition of claim 97 or claim 98, wherein the subunit of an HLA-1 protein comprises HLA-C, HLA-E, or HLA-G.

100. The composition of claim 99, wherein the subunit of an HLA-1 protein comprises HLA-E or HLA-G.

101. The composition of claim 99, wherein the subunit of an HLA-1 protein comprises HLA-E.

102. The composition of claim 99, wherein the subunit of an HLA-1 protein comprises HLA-G.

103. The composition of any one of claims 90 through 102, further comprising a third cell population wherein the third cell population does not contain a modified human cell of either the first or the second cell population.

104. The composition of claim 103, wherein the third cell population comprises at least 1, 2, 3, 4, 5, 6, 7, 8, 9, 10, 15, 20, 25, 30, 35, 40, 45, 50, 55, 60, 65, or 70% and/or no more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations, preferably 1-10%, more preferably 1-20%, even more preferably 1-30%, yet even more preferably 1-40%.

105. The composition of any one of claims 90 through 104, further comprising a fourth cell population wherein the fourth cell population does not contain a modified human cell of either the first, second, or third cell population.

106. The composition of claim 105, wherein the cell population comprises at least 1, 2, 3, 4, 5, 6, 7, 8, 9, 10, 15, 20, 25, 30, 35, 40, 45, 50, 55, 60, 65, or 70% and/or no more than 2, 3, 4, 5, 6, 7, 8, 9, 10, 15, 20, 25, 30, 35, 40, 45, 50, 55, 60, 65, or 75% of all of the cells in the plurality of cell populations, for example 1-75% of all the cells in the plurality of cell populations, preferably 1-10%, more preferably 1-20%, even more preferably 1-30%, yet even more preferably 1-40%.

107. The composition of any one of claims 90 to 106, wherein the cell populations comprise immune cells or stem cells.

108. The composition of claim 107, wherein the cell populations comprise immune cells comprising neutrophils, eosinophils, basophils, mast cells, monocytes, macrophages, dendritic cells, natural killer cells, or a lymphocytes.

109. The composition of claim 107, wherein the cell populations comprise immune cells comprising T cells.

110. The composition of claim 107, wherein the cell populations comprise stem cells comprising human pluripotent stem cells, multipotent stem cells, embryonic stem cells, induced pluripotent stem cells (iPSC), hematopoietic stem cells, or a CD34+ cells.

111. The composition of claim 107, wherein the cell populations comprise stem cells comprising induced pluripotent stem cells (iPSC).

112. A composition comprising a cell comprising a first nucleic acid-guided nuclease system comprising
(a) a first nucleic acid-guided nuclease comprising a Type V CRISPR endonuclease; and
(b) a first guide nucleic acid, compatible with the first nucleic acid-guided nuclease, comprising a spacer sequence directed at a first target nucleotide sequence in a gene coding for a first subunit of an HLA-1 protein;
wherein the first nucleic acid-guided nuclease and the first guide nucleic acid, when complexed, target and cleave at least one strand of DNA at a site at or near the first target nucleotide sequence in the gene coding for the first subunit of an HLA-1 protein.

113. The composition of claim 112, wherein the first subunit comprises B2M.

114. The composition of claim 112, wherein the cell further comprises a first donor template comprising a polynucleotide coding for a fusion protein comprising B2M and a second subunit of an HLA-1 protein.

115. The composition of claim 114, wherein the second subunit of an HLA-1 protein comprises HLA-C, HLA-E, or HLA-G.

116. The composition of claim 114, wherein the second subunit of an HLA-1 protein comprises HLA-E or HLA-G.

117. The composition of claim 114, wherein the second subunit of an HLA-1 protein comprises HLA-E.

118. The composition of claim 114, wherein the second subunit of an HLA-1 protein comprises HLA-G.

119. The composition of any one of claims 112 to 118, wherein the cell further comprises a second nucleic acid-guided nuclease system comprising
(c) a second nucleic acid-guided nuclease comprising a Type V CRISPR endonuclease; and
(d) a second guide nucleic acid, compatible with the second nucleic acid-guided nuclease, comprising a spacer sequence directed at a second target nucleotide sequence in a gene coding for a subunit of an HLA-2 protein or a transcription factor regulating the expression of one or more subunits of an HLA-2 protein;
wherein the second nucleic acid-guided nuclease and the second guide nucleic acid, when complexed, target and cleave at least one strand of DNA at a site at or near the second target nucleotide sequence in the gene coding for a subunit of an HLA-2 protein or a transcription factor regulating the expression of one or more subunits of an HLA-2 protein.

120. The composition of claim 119, wherein the transcription factor comprises CIITA.

121. The composition of any one of claims 112 to 120, wherein the cell further comprises a third nucleic acid-guided nuclease system comprising
(e) a third nucleic acid-guided nuclease comprising a Type V CRISPR endonuclease; and
(f) a third guide nucleic acid, compatible with the third nucleic acid-guided nuclease, comprising a spacer sequence directed at a third target nucleotide sequence in a gene coding for a subunit of a TCR protein;
wherein the third nucleic acid-guided nuclease and the third guide nucleic acid, when complexed, target and cleave at least one strand of DNA at a site at or near the third target nucleotide sequence in the gene coding for the subunit of a TCR protein.

122. The composition of claim 121, wherein the subunit of a TCR protein comprises an alpha subunit or a beta subunit or a TRAC, TRBC, CD3E, CD3D, CD3G, or CD3Z gene.

123. The composition of claim 122, wherein the subunit of a TCR protein is an alpha subunit.

124. The composition of claim 121, wherein the gene coding for the subunit of a TCR protein is a TRAC gene.

125. The composition of any one of claims 121 through 124, wherein the cell further comprises a donor template comprising a polynucleotide coding for a first chimeric antigen receptor (CAR) or portion thereof.

126. The composition of claim 125, wherein the CAR or portion thereof comprises a polypeptide that binds at least one of B7H3, BCMA, GPRC5D, CD8, CD8a, CD19, CD20, CD22, CD28, 4-1BB, or CD3zeta.

127. The composition of claim 126, wherein the CAR or portion thereof comprises a polypeptide at least 60, at least 70, at least 80, at least 90, at least 95, at least 99%, or 100% identical to any one of the amino acid sequences of SEQ ID NOs: 86-124.

128. The composition of claim 125, wherein the CAR or portion thereof comprises a polypeptide that binds at least one of B7H3, BCMxA, GPRC5D, CD8, CD8a, CD20, CD22, CD28, 4-1BB, or CD3zeta.

129. The composition of claim 128, wherein the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

130. A composition comprising a cell comprising a first nucleic acid-guided nuclease system comprising
(a) a first nucleic acid-guided nuclease comprising a Type V CRISPR endonuclease; and
(b) a first guide nucleic acid, compatible with the first nucleic acid-guided nuclease, comprising a spacer sequence directed at a first target nucleotide sequence in a gene coding for a subunit of an HLA-2 protein, or to a transcription factor regulating expression of one or more genes coding for one or more subunits of HLA-2 proteins;
wherein the first nucleic acid-guided nuclease and the first guide nucleic acid, when complexed, target and cleave at least one strand of DNA at a site at or near the first target nucleotide sequence in the gene coding for a subunit of an HLA-2 protein, or to a transcription factor regulating expression of one or more genes coding for one or more subunits of HLA-2 proteins.

131. The composition of claim 130, wherein the transcription factor comprises CIITA.

132. The composition of claim 130 or 131, wherein the cell further comprises a second nucleic acid-guided nuclease system comprising
(c) a second nucleic acid-guided nuclease comprising a Type V CRISPR endonuclease; and
(d) a second guide nucleic acid, compatible with the second nucleic acid-guided nuclease, comprising a spacer sequence directed at a second target nucleotide sequence in a gene coding for a subunit of a TCR protein;
wherein the second nucleic acid-guided nuclease and the second guide nucleic acid, when complexed, target and cleave at least one strand of DNA at a site at or near the second target nucleotide sequence in the gene coding for the subunit of a TCR protein.

133. The composition of claim 132, wherein the subunit of a TCR protein comprises an alpha subunit or a beta subunit or a TRAC, TRBC, CD3E, CD3D, CD3G, or CD3Z gene.

134. The composition of claim 133, wherein the subunit of a TCR protein is an alpha subunit.

135. The composition of claim 132, wherein the gene coding for the subunit of a TCR protein is a TRAC gene.

136. The composition of any one of claims 132 through 135, wherein the cell further comprises a donor template comprising a polynucleotide coding for a first chimeric antigen receptor (CAR) or portion thereof.

137. The composition of claim 136, wherein the CAR or portion thereof comprises a polypeptide that binds at least one of B7H3, BCMA, GPRC5D, CD8, CD8a, CD19, CD20, CD22, CD28, 4-1BB, or CD3zeta.

138. The composition of claim 137, wherein the CAR or portion thereof comprises a polypeptide at least 60, at least 70, at least 80, at least 90, at least 95, at least 99%, or 100% identical to any one of the amino acid sequences of SEQ ID NOs: 86-124.

139. The composition of claim 136, wherein the CAR or portion thereof comprises a polypeptide that binds at least one of B7H3, BCMxA, GPRC5D, CD8, CD8a, CD20, CD22, CD28, 4-1BB, or CD3zeta.

140. The composition of claim 139, wherein the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

141. A composition comprising a cell comprising a first nucleic acid-guided nuclease system comprising
(a) a first nucleic acid-guided nuclease comprising a Type V CRISPR endonuclease; and
(b) a first guide nucleic acid, compatible with the nucleic acid-guided nuclease, comprising a spacer sequence directed at a first target nucleotide sequence in a gene coding for a subunit of a TCR protein;
wherein the first nucleic acid-guided nuclease and the first guide nucleic acid, when complexed, target and cleave at least one strand of DNA at a site at or near the first target nucleotide sequence in the gene coding for the subunit of a TCR protein.

142. The composition of claim 141, wherein the subunit of a TCR protein comprises an alpha subunit or a beta subunit or a TRAC, TRBC, CD3E, CD3D, CD3G, or CD3Z gene.

143. The composition of claim 142, wherein the subunit of a TCR protein is an alpha subunit.

144. The composition of any one of claim 141, wherein the gene coding for the subunit of a TCR protein is a TRAC gene.

145. The composition of any one of claims 141 through 144, wherein the cell further comprises a donor template comprising a polynucleotide coding for a first chimeric antigen receptor (CAR) or portion thereof.

146. The composition of claim 145, wherein the CAR or portion thereof comprises a polypeptide that binds at least one of B7H3, BCMA, GPRC5D, CD8, CD8a, CD19, CD20, CD22, CD28, 4-1BB, or CD3zeta.

147. The composition of claim 146, wherein the CAR or portion thereof comprises a polypeptide at least 60, at least 70, at least 80, at least 90, at least 95, at least 99%, or 100% identical to any one of the amino acid sequences of SEQ ID NOs: 86-124.

148. The composition of claim 145, wherein the CAR or portion thereof comprises a polypeptide that binds at least one of B7H3, BCMxA, GPRC5D, CD8, CD8a, CD20, CD22, CD28, 4-1BB, or CD3zeta.

149. The composition of claim 148, wherein the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

150. The composition of any one of claims 112 to 149, wherein the nucleic acid-guided nuclease comprises an engineered, non-naturally occurring nuclease.

151. The composition of any one of claims 112 to 150, wherein the nucleic acid-guided nuclease comprises a Class 1 or a Class 2 nuclease.

152. The composition of claim 151, wherein the nucleic acid-guided nuclease comprises a Type II or a Type V nuclease.

153. The composition of claim 152, wherein the nucleic acid-guided nuclease comprises a Type V-A, V-B, V-C, V-D, or V-E nuclease.

154. The composition of claim 153, wherein the nucleic acid-guided nuclease comprises a Type V-A nuclease.

155. The composition of claim 154, wherein the nucleic acid-guided nuclease comprises a MAD nuclease, an ART nuclease, or an ABW nuclease.

156. The composition of claim 155, wherein the nucleic acid-guided nuclease comprises an amino acid sequence at least 80%, at least 85%, at least 90%, at least 95%, at least 99%, or 100% identical to an amino acid sequence of a MAD, ART, or ABW nuclease.

157. The composition of claim 155, wherein the nucleic acid-guided nuclease comprises a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20 nuclease.

158. The composition of claim 155, wherein the nucleic acid-guided nuclease comprises an ART1, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35 nuclease.

159. The composition of claim 155, wherein the nucleic acid-guided nuclease comprises an amino acid sequence at least 80%, at least 85%, at least 90%, at least 95%, at least 99%, or 100% identical, to the amino acid sequence of MAD2, MAD7, ART2, ART11, or ART11*.

160. The composition of claim 155, wherein the nucleic acid-guided nuclease comprises an amino acid sequence that is at least 80%, at least 85%, at least 90%, at least 95%, 99%, or 100% identical, to the amino acid sequence of SEQ ID NO: 37.

161. The composition of any one of claims 150 to 160, wherein the nucleic acid-guided nuclease further comprises at least one nuclear localization signal (NLS), at least one purification tag, and/or at least one cleavage site.

162. The composition of claim 161, wherein the nucleic acid-guided nuclease comprises at least 4 nuclear localization signals (NLS).

163. The composition of claim 162, wherein the nucleic acid-guided nuclease comprises one N-terminal and three C-terminal nuclease localization signals (NLS).

164. The composition of claim 161 through 163, wherein the nuclear localization signals comprise any one of SEQ ID NOs: 40-56.

165. The composition of claim 164, wherein the NLS comprises SEQ ID NOs: 40, 51, and 56.

166. The composition of any one of claims 112 to 165, wherein the guide nucleic acid comprises:
(i) a targeter nucleic acid comprising a targeter stem sequence and the spacer sequence; and
(ii) a modulator nucleic acid comprising a modulator stem sequence complementary to the targeter stem sequence, and, optionally, a 5′ sequence.

167. The composition of claim 166, wherein the guide nucleic acid comprises a single polynucleotide.

168. The composition of claim 166 or claim 167, wherein the guide nucleic acid comprises an engineered, non-naturally occurring guide nucleic acid.

169. The composition of claim 166 or claim 168, wherein the targeter nucleic acid and the modulator nucleic acid are separate polynucleotides.

170. The composition of claim 169, wherein the dual guide nucleic acid is capable of binding to and activating a nucleic acid-guided nuclease, that, in a naturally occurring system, is activated by a single crRNA in the absence of a tracrRNA.

171. The composition of any one of claims 112 through 170, wherein the guide nucleic acid further comprises a donor template recruiting sequence.

172. The composition of any one of claims 112 through 171, wherein the target nucleotide sequence is within at least 10, 20, 30, 40, or 50 nucleotides of a protospacer adjacent motif (PAM) that is recognized by the nucleic acid-guided nuclease.

173. The composition of any one of claims 166 through 172, wherein the guide nucleic acid comprises a spacer sequence at least 80%, at least 85%, at least 90%, at least 95%, at least 99.5%, or 100% identical to any one of any one of SEQ ID NOs: 125-2019.

174. The composition of any one of claims 112 through 173, wherein some or all of the guide nucleic acid comprises RNA.

175. The composition of claim 174, wherein at least 50%, at least 70%, at least 90%, at least 95%, or 100% of the guide nucleic acid comprises RNA.

176. The composition of any one of claims 112 through 175, wherein the guide nucleic acid comprises one or more chemical modifications to one or more nucleotides and/or internucleotide linkages at or near the 5′ end, at or near the 3′ end, and/or both.

177. The composition of claim 176, wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, or a combination thereof.

178. The composition of any one of claims 112 through 177, wherein the donor template comprises single-stranded DNA, linear single-stranded RNA, linear double-stranded DNA, linear double-stranded RNA, circular single-stranded DNA, circular single-stranded RNA, circular double-stranded DNA, or circular double-stranded RNA.

179. The composition of any one of claims 114 through 118, 125 through 129, 136 through 140, or 145 through 149, wherein the donor template comprises two homology arms.

180. The composition of claim 179, wherein the homology arms comprise at least 50, 100, 150, 200, 250, 300, 350, 400, 450, 500, 600, 700, 800, or 900 and/or at most 100, 150, 200, 250, 300, 350, 400, 450, 500, 600, 700, 800, 900, or 1000 nucleotides, for example 50-1000 nucleotides, preferably 100-800 nucleotides, more preferably 250-750 nucleotides, even more preferably 400-600 nucleotides.

181. The composition of any one of claims 114 through 118, 125 through 129, 136 through 140, or 145 through 149, wherein the donor template comprises a mutation in a PAM sequence to partially or completely abolish binding of the RNP to the DNA.

182. The composition of any one of claims 114 through 118, 125 through 129, 136 through 140, or 145 through 149, wherein the donor template comprises one or more promoters.

183. The composition of claim 182, wherein the promoter shares at least 70%, at least 75%, at least 80%, at least 85%, at least 90%, at least 95%, at least 99.5%, or 100% sequence identity with any one of SEQ ID NOs: 78-85.

184. The composition of any one of claims 114 through 118, 125 through 129, 136 through 140, or 145 through 149, wherein the donor template comprises one or more chemical modifications to one or more nucleotides and/or internucleotide linkages at or near the 5′ end, at or near the 3′ end, or both.

185. The composition of claim 184, wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, a suitable alternative, or a combination thereof.

186. The composition of any one of claims 112 through 185, wherein the cell comprises an immune cell or a stem cell.

187. The composition of claim 186, wherein the cell comprises an immune cell comprising a neutrophil, eosinophil, basophil, mast cell, monocyte, macrophage, dendritic cell, natural killer cell, or a lymphocyte.

188. The composition of claim 186, wherein the cell comprises a T cell.

189. The composition of claim 186, wherein the cell comprises a stem cell comprising a human pluripotent, multipotent stem cell, embryonic stem cell, induced pluripotent stem cell, hematopoietic stem cell, or a CD34+ cell.

190. The composition of claim 186, wherein the cell comprises a stem cell comprising an iPSC.

191. A composition comprising (a) a first guide nucleic acid comprising a spacer sequence complementary to a target nucleotide sequence within a B2M gene;
(b) a second guide nucleic acid comprising a spacer sequence complementary to a target nucleotide sequence within a CIITA gene;
(c) a third guide nucleic acid comprising a spacer sequence complementary to a target nucleotide sequence within a TCR subunit gene; and
(d) one or more nucleic acid-guided nucleases optionally complexed with one or more of the guide nucleic acids of (a), (b), or (c).

192. The composition of claim 191, wherein the gene coding for a subunit of a TCR is a TRAC gene or a TRAC, TRBC, CD3E, CD3D, CD3G, or CD3Z gene.

193. The composition of claim 191 or 192, wherein the one or more nucleic acid-guided nucleases comprise Class 1 or a Class 2 nucleases.

194. The composition of claim 193, wherein the one or more nucleic acid-guided nucleases comprise Type II or a Type V nuclease.

195. The composition of claim 193, wherein the one or more nucleic acid-guided nucleases comprise Type V-A, V-B, V-C, V-D, or V-E nucleases.

196. The composition of claim 193, wherein the one or more nucleic acid-guided nucleases comprise Type V-A nucleases.

197. The composition of claim 193, wherein the one or more nucleic acid-guided nucleases comprise a MAD nuclease, an ART nuclease, or an ABW nuclease.

198. The composition of claim 193, wherein the one or more nucleic acid-guided nucleases each comprise an amino acid sequence at least 80%, at least 85%, at least 90%, at least 95%, or at least 99% identical to the amino acid sequence of MAD, ART, or ABW nuclease.

199. The composition of claim 193, wherein the one or more nucleic acid-guided nucleases each comprise a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20 nuclease.

200. The composition of claim 193, wherein the one or more nucleic acid-guided nucleases each comprise an ART1, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35 nuclease.

201. The composition of claim 193, wherein the one or nucleic acid-guided nucleases each comprise an amino acid sequence at least 80%, at least 85%, at least 90%, at least 95%, or at least 99% identical to the amino acid sequence of MAD2, MAD7, ART2, ART11, or ART11*.

202. The composition of any one of claims 191 through 201, wherein the first, second, and/or third guide nucleic acids comprise:
(i) a targeter nucleic acid comprising a targeter stem sequence and a spacer sequence; and
(ii) a modulator nucleic acid comprising a modulator stem sequence complementary to the targeter stem sequence, and, optionally, a 5′ sequence.

203. The composition of claim 202, wherein the targeter nucleic acid and the modulator nucleic acid comprise a single polynucleotide.

204. The composition of claim 202 or claim 203, wherein the guide nucleic acid comprises an engineered, non-naturally occurring guide nucleic acid.

205. The composition of claim 202 or claim 204, wherein the guide nucleic acid comprises a dual guide nucleic acid, wherein the targeter nucleic acid and the modulator nucleic acid are separate polynucleotides.

206. The composition of claim 205, wherein the dual guide nucleic acid is capable of binding to and activating a nucleic acid-guided nuclease, that, in a naturally occurring system, is activated by a single crRNA in the absence of a tracrRNA.

207. The composition of any one of claims 202 through 206, wherein the target nucleotide sequence is within at least 10, 20, 30, 40, or 50 nucleotides of a protospacer adjacent motif (PAM) that is recognized by the nucleic acid-guided nuclease.

208. The composition of any one of claims 202 through 207, wherein the guide nucleic acid further comprises a donor template recruiting sequence.

209. The composition of any one of claims 202 through 208, wherein the guide nucleic acid comprises a spacer sequence is at least 80%, at least 85%, at least 90%, at least 95%, at least 99.5%, or 100% identical to any one of any one of SEQ ID NOs: 125-2019.

210. The composition of any one of claims 202 through 209, wherein some or all of the guide nucleic acid is RNA.

211. The composition of claim 210, wherein at least 50%, at least 70%, at least 90%, at least 95%, or 100% of the guide nucleic acid comprises RNA.

212. The composition of any one of claims 202 through 211, wherein the guide nucleic acid comprises one or more chemical modifications to one or more nucleotides and/or internucleotide linkages at or near the 5′ end, at or near the 3′ end, and/or both.

213. The composition of claim 212, wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, a suitable alternative, or a combination thereof.

214. The composition of any one of claims 191 to 213, further comprising:
(c) a first donor template comprising a first transgene.

215. The composition of claim 214, wherein the first transgene comprises a polynucleotide encoding a fusion protein comprising B2M and HLA-A, -B, -C, -D, -E, -F, or -G.

216. The composition of claim 215, wherein the fusion protein comprises HLA-C, -E, or -G.

217. The composition of claim 216, wherein the fusion protein comprises HLA-E or HLA-G.

218. The composition of claim 217, wherein the fusion protein comprises HLA-E.

219. The composition of claim 217, wherein the fusion protein comprises HLA-G.

220. The composition of any one of claims 214 to 219, wherein the first donor template comprises homology arms, wherein the first homology arm is complementary to a region upstream and the second homology arm is complementary to a region downstream of a cleavage site within a B2M gene.

221. The composition of any one of claims 191 through 220, further comprising (f) a second donor template comprising a second transgene.

222. The composition of claim 221, wherein the second transgene comprises a first portion of a polynucleotide coding for a first chimeric antigen receptor (CAR).

223. The composition of claim 222, wherein the CAR or portion thereof comprises a polypeptide that binds at least one of B7H3, BCMA, GPRC5D, CD8, CD8a, CD19, CD20, CD22, CD28, 4-1BB, or CD3zeta.

224. The composition of claim 223, wherein the CAR or portion thereof comprises a polypeptide at least 60, at least 70, at least 80, at least 90, at least 95, at least 99%, or 100% identical to any one of the amino acid sequences of SEQ ID NOs: 86-124.

225. The composition of claim 221, wherein the CAR or portion thereof comprises a polypeptide that binds at least one of B7H3, BCMA, GPRC5D, CD8, CD8a, CD20, CD22, CD28, 4-1BB, or CD3zeta.

226. The composition of claim 225, wherein the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

227. The composition of any one of claims 222 through 226, further comprising a second portion of the polynucleotide, wherein the second portion codes for a second CAR or portion thereof, different from the first CAR or portion thereof.

228. The composition of any one of claims 221 to 227, wherein the second donor template comprises homology arms, wherein the first homology arm is complementary to a region upstream and the second homology arm is complementary to a region downstream of a cleavage site within a TRC subunit gene.

229. The composition of any one of claims 191 through 228, further comprising (g) a third donor template comprising a third transgene.

230. The composition of any one of claims 214 to 229, wherein the donor template comprises single-stranded DNA, linear single-stranded RNA, linear double-stranded DNA, linear double-stranded RNA, circular single-stranded DNA, circular single-stranded RNA, circular double-stranded DNA, or circular double-stranded RNA.

231. The composition of any one of claims 214 to 230, wherein the donor template comprises a mutation in a PAM sequence to partially or completely abolish binding of the RNP to the DNA.

232. The composition of any one of claims 214 to 231, wherein the donor template comprises one or more promoters.

233. The composition of claim 232, wherein the promoter shares at least 70%, at least 75%, at least 80%, at least 85%, at least 90%, at least 95%, at least 99.5% sequence identity with any one of SEQ ID NOs: 78-85.

234. The composition of any one of claims 214 to 233, wherein the donor template comprises one or more chemical modifications to one or more nucleotides and/or internucleotide linkages at or near the 5′ end, at or near the 3′ end, or both.

235. The composition of claim 234, wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, a suitable alternative, or a combination thereof.

236. A modified cell that
(a) partially or completely lacks cell surface-expressed
(i) active HLA-1 protein;
(ii) active HLA-2 protein; or
(iii) active TCR protein; and
(b) comprises one or more
(i) CAR proteins expressed on the cell surface; and
(ii) fusion proteins comprising HLA-E or HLA-G expressed on the cell surface.

237. The modified cell of 236, wherein the cell comprises a human cell.

238. The modified cell of 237, wherein the human cell comprises an immune cell or a stem cell.

239. The modified cell of 238, wherein the immune cell comprises a neutrophil, eosinophil, basophil, mast cell, monocyte, macrophage, dendritic cell, natural killer cell, or a lymphocyte.

240. The modified cell of 238, wherein the immune cell comprises a T cell.

241. The modified cell of 238, wherein the stem cell comprises a human pluripotent, multipotent stem cell, embryonic stem cell, induced pluripotent stem cell, hematopoietic stem cell, CD34+ cell.

242. A human cell comprising:
(a) a first, and optionally a second and/or third nucleic acid-guided nuclease, wherein at least one of the nucleases comprises a CRISPR endonuclease; and
(b) at least one of
(i) a first guide nucleic acid directed at a first target nucleotide sequence in a gene coding for a subunit of an HLA-1 protein;
(ii) a second guide nucleic acid directed at a second target nucleotide sequence in a gene coding for a subunit of an HLA-2 protein or a transcription factor for one or more genes coding for a subunit of an HLA-2 protein; and
(iii) a third guide nucleic acid directed at a third target nucleotide sequence coding for a subunit of a TCR.

243. The human cell of claim 242, further comprising:
(c) a donor template comprising a polynucleotide coding for a chimeric antigen receptor (CAR) protein or part of a CAR.

244. The human cell of claim 243, wherein the protein comprises a protein directed at B7H3, BCMA, GPRC5D, CD19, CD20, CD22, or a combination thereof.

245. The human cell of claim 244, wherein the CAR or portion thereof comprises a polypeptide at least 60, at least 70, at least 80, at least 90, at least 95, at least 99%, or 100% identical to any one of the amino acid sequences of SEQ ID NOs: 86-124.

246. The human cell of any one of claims 243 through 245, wherein the donor template comprises homology arms for insertion at a cleavage site in the subunit of the TCR to which the guide nucleic acid is directed.

247. The human cell of any one of claims 242 to 243, further comprising:
(d) a donor template comprising a polynucleotide coding an HLA-A, HLA-B, HLA-C, HLA-D, HLA-E, HLA-F, or HLA-G protein.

248. The human cell of any one of claims 242 to 247, wherein the human cell comprises an immune cell or a stem cell.

249. The human cell of claim 248, wherein the human cell comprises an immune cell comprising a neutrophil, eosinophil, basophil, mast cell, monocyte, macrophage, dendritic cell, natural killer cell, or a lymphocyte.

250. The human cell of claim 248, wherein the human cell comprises an immune cell comprising a T cell.

251. The human cell of claim 248, wherein human cell comprises a stem cell comprising a human pluripotent, multipotent stem cell, embryonic stem cell, induced pluripotent stem cell, hematopoietic stem cell, CD34+ cell.

252. The human cell of claim 251, wherein human cell comprises a stem cell comprising an induced pluripotent stem cell.

253. A modified human cell comprising (a) reduced or eliminated B2M and knock-in of HLA-E or HLA-G; or
(b) reduced or eliminated TCR and knock-in.

254. The modified human cell of claim 253, wherein the human cell comprises an immune cell or a stem cell.

255. The modified human cell of 254, wherein the human cell comprises an immune cell comprising a neutrophil, eosinophil, basophil, mast cell, monocyte, macrophage, dendritic cell, natural killer cell, or a lymphocyte.

256. The modified human cell of 254, wherein the human cell comprises an immune cell comprising a T cell.

257. The modified human cell of 254, wherein the human cell comprises a stem cell comprising a human pluripotent, multipotent stem cell, embryonic stem cell, induced pluripotent stem cell, hematopoietic stem cell, CD34+ cell.

258. The modified human cell of 254, wherein the human cell comprises an induced pluripotent stem cell.

259. A human stem cell comprising:
(a) a first genomic modification in an endogenous B2M gene that partially or completely eliminates expression of the endogenous B2M;
(b) a second genomic modification in a CIITA gene that partially or completely eliminates expression of the CIITA; and
(c) a third genomic modification in a TCR subunit gene that partially or completely eliminates expression of the TCR subunit.

260. The human stem cell of claim 259, wherein the cell comprises an iPSC.

261. The human stem cell of claim 259 or 260, further comprising:
(d) an exogenous polynucleotide encoding for a fusion protein comprising one or more HLA-A, -B, -C, -D, -E, -F, or -G protein inserted into the B2M gene.

262. The human stem cell of any of claims 259 to 261, further comprising
(e) an exogenous polynucleotide encoding for one or more CARs inserted into the TCR subunit gene.

263. The human stem cell of claim 262, wherein the CAR or portion thereof comprises a polypeptide that binds at least one of BCMA, GPRC5D, CD8, CD8a, CD19, CD20, CD22, CD28, 4-1BB, or CD3zeta.

264. A method for treating a disorder comprising administering to an individual suffering from a disorder an effective amount of a composition comprising a composition of any one of the claims 1 through 190 or 236 through 263.

265. A method of producing a non-immunogenic CAR T cell comprising:
(a) modifying a genome of a cell to reduce or eliminate cell surface expression of active HLA-1 proteins in the cell and its progeny;
(b) introducing into the genome of the cell or one or more of its progeny a first polynucleotide coding for surface expression of a first CAR or portion thereof specific for a first antigen; and
(c) introducing into the genome of the cell or one or more of its progeny a second polynucleotide coding for surface expression of a second CAR or portion thereof specific for a second antigen.

266. The method of claim 265, wherein modifying genome of a cell to reduce or eliminate cell surface expression of active HLA-1 proteins comprises introducing a genomic modification into a B2M gene that partially or completely inactivates the B2M gene.

267. The method of claim 266, wherein modifying the genome comprises introducing a substitution, an insertion, a deletion, a nonsense mutation, or a truncation.

268. The method of claim 267, wherein the genomic modification comprises inserting a first transgene into a site within the B2M gene, wherein the first transgene codes for a B2M-HLA subunit fusion protein.

269. The method of claim 268, wherein the HLA subunit of the B2M-HLA subunit fusion protein comprises an HLA-C, -E, or -G subunit.

270. The method of claim 268, wherein the HLA subunit of the B2M-HLA subunit fusion protein comprises an HLA-E or -G subunit.

271. The method of claim 268, wherein the HLA subunit of the B2M-HLA subunit fusion protein comprises an HLA-E.

272. The method of claim 268, wherein the HLA subunit of the B2M-HLA subunit fusion protein comprises an HLA-G.

273. The method of any one of claims 265 through 272, wherein the first and/or second CAR or portion thereof comprises a CAR or portion thereof that binds B7H3, BCMA, GPRC5D, CD8, CD8a, CD19, CD20, CD22, CD28, 4-1BB, or CD3zeta.

274. The method of claim 273, wherein the CAR or portion thereof comprises a polypeptide at least 60, at least 70, at least 80, at least 90, at least 95, at least 99%, or 100% identical to any one of the amino acid sequences of SEQ ID NOs: 86-124.

275. The method of any one of claims 265 through 272, wherein the first and/or second CAR or portion thereof comprises a CAR or portion thereof that binds B7H3, BCMA, GPRC5D, CD8, CD8a, CD20, CD22, CD28, 4-1BB, or CD3zeta.

276. The method of claim 275, wherein the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

277. The method of any one of claims 265 through 276, wherein the polynucleotide coding for surface expression of a CAR is introduced at a site with a TCR subunit gene or a safe harbor site.

278. The method of any one of claims 265 through 277, further comprising:
(d) modifying the genome of the cell or one of its progeny to reduce or eliminate cell surface expression of one or more subunits of an HLA-2 protein.

279. The method of claim 278, wherein modifying a genome of the cell or one of its progeny to reduce or eliminate cell surface expression of one or more subunits of an HLA-2 protein comprises introducing a genomic modification into a gene coding for a transcription factor for one or more genes encoding the one or more subunits of an HLA-2 protein that partially or completely inactivates the gene for the transcription factor.

280. The method of claim 279, wherein the genomic modification comprises a substitution, an insertion, a deletion, a nonsense mutation, or a truncation.

281. The method of claim 279 or claim 280, wherein the transcription factor comprises CIITA.

282. The method of any one of claims 268 to 281, wherein introducing into the genome comprises delivering into the cell a nucleic acid-guided nuclease system, or one or more polynucleotides encoding for one or more parts of the system, comprising:
(i) a nucleic acid-guided nuclease; and
(ii) a guide nucleic acid compatible with and capable of binding to and activating the nucleic acid-guided nuclease, wherein the guide nucleic acid comprises:
(1) a targeter nucleic acid comprising a targeter stem sequence and a spacer sequence, wherein the spacer sequence is complementary to a target nucleotide sequence within a target polynucleotide of a genome of a human target cell; and
(2) a modulator nucleic acid comprising a modulator stem sequence complementary to the targeter stem sequence, and, optionally, a 5′ sequence;
wherein the nucleic acid-guided nuclease system target and cleave at least one strand in the target polynucleotide at or near the target nucleotide sequence.

283. The method of claim 282, wherein the nucleic acid-guided nuclease comprises a Class 1 or a Class 2 nuclease.

284. The method of claim 283, wherein the nucleic acid-guided nuclease comprises a Type II or a Type V nuclease.

285. The method of claim 284, wherein the nucleic acid-guided nuclease comprises a Type V-A, V-B, V-C, V-D, or V-E nuclease.

286. The method of claim 285, wherein the nucleic acid-guided nuclease comprises a Type V-A nuclease.

287. The method of claim 286, wherein the nucleic acid-guided nuclease comprises a MAD nuclease, an ART nuclease, or an ABW nuclease.

288. The method of claim 286, wherein the nucleic acid-guided nuclease comprises an amino acid sequence at least 80%, at least 85%, at least 90%, at least 95%, at least 99%, or 100% identical to the amino acid sequence of MAD, ART, or ABW nuclease.

289. The method of claim 286, wherein the nucleic acid-guided nuclease comprises a MAD1, MAD2, MAD3, MAD4, MADS, MAD6, MAD7, MAD8, MAD9, MAD10, MAD11, MAD12, MAD13, MAD14, MAD15, MAD16, MAD17, MAD18, MAD19, or MAD20 nuclease.

290. The method of claim 286, wherein the nucleic acid-guided nuclease comprises an ART1, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35 nuclease.

291. The method of claim 286, wherein the nucleic acid-guided nuclease comprises an amino acid sequence at least 80%, at least 85%, at least 90%, at least 95%, at least 99%, or 100% identical to the amino acid sequence of MAD2, MAD7, ART2, ART11, or ART11*.

292. The method of claim 286, wherein the nucleic acid-guided nuclease comprises an amino acid sequence that is at least 80%, at least 85%, at least 90%, at least 95%, 99%, or 100% identical, to the amino acid sequence of SEQ ID NO: 37.

293. The method of any one of claims 282 through 292, wherein the nucleic acid-guided nuclease comprises at least one nuclear localization signal (NLS), at least one purification tag, or at least one cleavage site.

294. The method of claim 293, wherein the nucleic acid-guided nuclease comprises at least 4 NLS.

295. The method of claim 294, wherein the nucleic acid-guided nuclease comprises one N-terminal and three C-terminal nuclease localization signals (NLS).

296. The method of any one of claims 293 through 295, wherein the nuclear localization signals comprise any one of SEQ ID NOs: 40-56.

297. The method of claim 296, wherein the NLS comprises SEQ ID NOs: 40, 51, and 56.

298. The method of claim 282 through 297, wherein the guide nucleic acid comprises a single polynucleotide.

299. The method of claim 282 through 297, wherein the guide nucleic acid comprises a dual guide nucleic acid, wherein the targeter nucleic acid and the modulator nucleic acid are separate polynucleotides.

300. The method of claim 299, wherein the dual guide nucleic acid is capable of binding to and activating a nucleic acid-guided nuclease, that, in a naturally occurring system, is activated by a single crRNA in the absence of a tracrRNA.

301. The method of claim 282 through 300, wherein the target nucleotide sequence is within at least 10, at least 20, at least 30, at least 40, or at least 50 nucleotides of a protospacer adjacent motif (PAM) that is recognized by a nuclease with which the guide nucleic acid is compatible.

302. The method of claim 282 through 301, wherein the guide nucleic acid and the nuclease form a nucleic acid-guided nuclease complex.

303. The method of claim 302, wherein the guide nucleic acid further comprises a donor template recruiting sequence.

304. The method of claim 282 through 303, wherein the guide nucleic acid comprises a spacer sequence is at least 80%, at least 85%, at least 90%, at least 95%, at least 99.5%, or 100% identical to any one of any one of SEQ ID NOs: 125-2019.

305. The method of claim 282 through 304, wherein some or all of the guide nucleic acid is RNA.

306. The method of claim 305, wherein at least 50%, at least 70%, at least 90%, at least 95%, or 100% of the guide nucleic acid comprises RNA.

307. The method of claim 282 through 306, wherein the guide nucleic acid comprises one or more chemical modifications to one or more nucleotides and/or internucleotide linkages at or near the 5′ end, at or near the 3′ end, and/or both.

308. The method of claim 307, wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, a suitable alternative, or a combination thereof.

309. The method of claim 282 through 308, wherein introducing into the genome further comprises delivering a donor template comprising the transgene.

310. The method of claim 309, wherein the donor template comprises two homology arms flanking the transgene.

311. The method of claim 310, wherein the homology arms comprise at most 1000, at most 900, at most 800, at most 700, at most 600, at most 500 nucleotides.

312. The method of any one of claims 309 through 311, wherein the donor template comprises single-stranded DNA, linear single-stranded RNA, linear double-stranded DNA, linear double-stranded RNA, circular single-stranded DNA, circular single-stranded RNA, circular double-stranded DNA, or circular double-stranded RNA.

313. The method of any one of claims 309 through 312, wherein the donor template comprises a mutation in a PAM sequence to partially or completely abolish binding of the RNP to the DNA.

314. The method of any one of claims 309 through 313, wherein the donor template comprises one or more promoters.

315. The method of claim 314, wherein the promoter shares at least 70%, at least 75%, at least 80%, at least 85%, at least 90%, at least 95%, at least 99.5%, or 100% sequence identity with any one of SEQ ID NOs: 78-85.

316. The method of any one of claims 309 through 315, wherein the donor template comprises one or more chemical modifications to one or more nucleotides and/or internucleotide linkages at or near the 5′ end, at or near the 3′ end, and/or both.

317. The method of claim 316, wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, a suitable alternative, or a combination thereof.

318. The method of any one of claims 309 through 317, wherein at least portion of the donor template is inserted by an innate cell repair mechanism at or near the strand break.

319. The method of claim 318, wherein the innate cell repair mechanism comprises homology directed repair (HDR).

320. The method of any one of claims 265 to 319, wherein the cell comprises a human cell.

321. The method of claim 320, wherein the human cell comprises an immune cell or a stem cell.

322. The method of claim 321, wherein the human cell comprises an immune cell comprising a neutrophil, eosinophil, basophil, mast cell, monocyte, macrophage, dendritic cell, natural killer cell, or a lymphocyte.

323. The method of claim 321, wherein the human cell comprises an immune cell comprising a T cell.

324. The method of claim 321, wherein the human cell comprises a stem cell comprising a human pluripotent, multipotent stem cell, embryonic stem cell, induced pluripotent stem cell, hematopoietic stem cell, CD34+ cell.

325. The method of claim 321, wherein the human cell comprises a stem cell comprising an induced pluripotent stem cell.

326. The method of any one of claims 268 to 325, wherein delivering comprises electroporation.

327. A method for producing a population of non-immunogenic CAR T cells comprising:
(a) modifying a genome of a first cell to reduce or eliminate cell surface expression of HLA-1 proteins in the first cell and its progeny;
(b) introducing into the genome of the first cell a first polynucleotide coding for surface expression of a first CAR specific for a first antigen on the first cell;
(c) modifying a genome of a second cell to reduce or eliminate cell surface expression of HLA-1 proteins in the second cell and its progeny; and
(d) introducing into the genome of the second cell a second polynucleotide coding for surface expression of a second CAR specific for a second antigen on the second cell, wherein the first and second cells are the same cell, the first cell is a progeny of the second cell, or the second cell is a progeny of the first cell.

328. A method of producing a cell with an engineered genome comprising
(a) modifying a B2M gene in the genome of a first cell to reduce or eliminate expression of the B2M gene;
(b) modifying a T cell receptor (TCR) subunit gene in the genome of a second cell to reduce or eliminate expression of the subunit;
(c) modifying a CIITA gene in the genome of a third cell to reduce or eliminate expression of the CIITA gene; and
(d) introducing a first transgene into the genome of a fourth cell, wherein the first transgene codes for a B2M-HLA subunit fusion protein.

329. The method of claim 328, wherein (a) through (d) are performed simultaneously, wherein the first, second, third, and fourth cells are the same cell.

330. The method of claim 328, wherein one or more of (a) through (d) are performed sequentially.

331. The method of claim 330, wherein one or more cells resulting from claim 330 are propagated prior to performing the remainder of (a) through (d) not performed in claim 330.

332. The method of any one of claims 328 through 331, wherein the TCR subunit comprises an alpha subunit or a beta subunit or a TRAC, TRBC, CD3E, CD3D, CD3G, or CD3Z gene.

333. The method of claim 332, wherein the TCR subunit comprises an alpha subunit.

334. The method of any one of claims 328 to 333, wherein the HLA subunit of the B2M-HLA subunit fusion protein comprises an HLA-C, -E, or -G subunit.

335. The method of claim 334, wherein the HLA subunit of the B2M-HLA subunit fusion protein comprises an HLA-E or -G subunit.

336. The method of claim 334, wherein the HLA subunit of the B2M-HLA subunit fusion protein comprises an HLA-E.

337. The method of claim 334, wherein the HLA subunit of the B2M-HLA subunit fusion protein comprises an HLA-G.

338. The method of any one of claims 328 to 337, wherein the first transgene is introduced at a site within the B2M gene.

339. The method of any one of claims 328 to 338, wherein the cell comprises a human cell.

340. The method of claim 339, wherein the human cell comprises an immune cell or a stem cell.

341. The method of claim 340, wherein the human cell comprises an immune cell comprising a neutrophil, cosinophil, basophil, mast cell, monocyte, macrophage, dendritic cell, natural killer cell, or a lymphocyte.

342. The method of claim 340, wherein the human cell comprises an immune cell comprising a T cell.

343. The method of claim 340, wherein the human cell comprises a stem cell comprising a human pluripotent, multipotent stem cell, embryonic stem cell, induced pluripotent stem cell, hematopoietic stem cell, CD34+ cell.

344. The method of claim 340, wherein the human cell comprises a stem cell comprising an induced pluripotent stem cell.

345. The method of any one of claims 328 to 344, further comprising:
(c) introducing a second transgene into the genome, wherein the second transgene codes for a chimeric antigen receptor (CAR) or portion thereof.

346. The method of claim 345, wherein the second transgene is introduced at a site within the TCR subunit gene.

347. The method of any one of claims 345 to 346, wherein the CAR or portion thereof comprises polypeptide that binds to B7H3, BCMA, GPRC5D, CD8, CD8a, CD19, CD20, CD22, CD28, 4-1BB, or CD3zeta.

348. The method of claim 347, wherein the CAR or portion thereof comprises a polypeptide at least 60, at least 70, at least 80, at least 90, at least 95, at least 99%, or 100% identical to any one of the amino acid sequences of SEQ ID NOs: 86-124.

349. The method of any one of claims 345 to 346, wherein the CAR or portion thereof comprises a polypeptide that binds at least one of B7H3, BCMA, GPRC5D, CD8, CD8a, CD20, CD22, CD28, 4-1BB, or CD3zeta.

350. The method of claim 349, wherein the CAR or portion thereof comprises a polypeptide at least 60, at least 70, at least 80, at least 90, at least 95, at least 99%, or 100% identical to any one of the amino acid sequences of SEQ ID NOs: 86-104 or 116-124.

351. The method of any one of claims 328 to 350, wherein the modifying of step (a) comprises contacting DNA of the genome with a first nucleic acid-guided nuclease complexed with a first compatible guide nucleic acid (gNA) targeted to a first target nucleotide sequence within the B2M gene so that the DNA is cleaved at or near the first target nucleotide sequence.

352. The method of any one of claims 328 to 351, wherein the modifying of step (b) comprises contacting DNA of the genome with a second nucleic acid-guided nuclease complexed with a second compatible guide nucleic acid targeted to a second target nucleotide sequence within the ‘gene so that the DNA is cleaved at or near the second target nucleotide sequence.

353. The method of anyone of claims 328 to 352, wherein the modifying of step (c) comprises contacting DNA of the genome with a third nucleic acid-guided nuclease complexed with a third compatible guide nucleic acid targeted to a third target nucleotide sequence within the CIITA subunit gene so that the DNA is cleaved at or near the third target nucleotide sequence.

354. A method of modifying a genome of a human cell comprising:
(a) modifying a B2M gene in the genome to reduce or eliminate expression of the B2M gene;
(b) modifying a T cell receptor (TCR) subunit gene in the genome to reduce or eliminate expression of the subunit; and
(c) modifying a CIITA gene in the genome to reduce or eliminate expression of the CIITA gene;
wherein at least 2 of (a) to (c) are performed sequentially, not simultaneously, thereby producing a modified human cell.

355. A composition comprising a modified human cell comprising:
(a) a first genomic modification comprising a first portion of a first polynucleotide, wherein the first portion comprises a transgene, inserted into a site with a TRC subunit gene, whereby the TRC subunit gene is partially or completely inactivated and the transgene is expressed; and
(b) a second genomic modification comprising a second polynucleotide coding for a fusion protein of B2M and HLA-E or HLA-G inserted into a B2M gene, whereby endogenous B2M is partially or completely inactivated and the fusion protein is expressed.

356. The composition of claim 355, wherein the TRC subunit gene is completely inactivated.

357. The composition of claim 355 or claim 356, wherein the endogenous B2M gene is completely inactivated.

358. The composition of claim 355, further comprising:
(c) a third genomic modification in a CIITA gene, wherein the CIITA gene is partially or completely inactivated.

359. The composition of claim 358, wherein the CIITA gene is completely inactivated.

360. The composition of any one of claims 355-359, wherein the TRC subunit gene comprises a TRAC, TRBC, CD3E, CD3D, CD3G, or CD3Z gene.

361. The composition of claim 360, wherein the TRC subunit gene comprises a TRAC gene.

362. The composition of claim 360, wherein the TRC subunit gene comprises a TRBC gene.

363. The composition of claim 360, wherein the TRC subunit gene comprises a CD3E gene.

364. The composition of claim 360, wherein the TRC subunit gene comprises a CD3D gene.

365. The composition of claim 360, wherein the TRC subunit gene comprises a CD3G gene.

366. The composition of claim 360, wherein the TRC subunit gene comprises a CD3Z gene.

367. The composition of any one of claims 355-366, wherein the transgene comprises a CAR or portion thereof, a cytokine, and/or a reporter gene.

368. The composition of claim 367, wherein the transgene comprises a CAR or portion thereof.

369. A composition comprising a modified human cell comprising:
(a) a first genomic modification comprising a first portion of a polynucleotide, wherein the first portion comprises a transgene, inserted into a site with a TRC subunit gene, whereby the TRC subunit gene is partially or completely inactivated and the transgene is expressed; and
(b) a second genomic modification in a CIITA gene, wherein the CIITA gene is partially or completely inactivated.

370. The composition of claim 369, wherein the TRC subunit gene is completely inactivated.

371. The composition of claim 369 or claim 356, wherein the CIITA gene is completely inactivated.

372. The composition of any one of claims 369-371, further comprising:
(c) a third genomic modification comprising a polynucleotide coding for a fusion protein of B2M and HLA-E or HLA-G inserted into a B2M gene, whereby endogenous B2M is partially or completely inactivated and the fusion protein is expressed.

373. The composition of claim 372, wherein endogenous B2M is completely inactivated.

374. The composition of any one of claims 369-373, wherein the TRC subunit gene comprises a TRAC, TRBC, CD3E, CD3D, CD3G, or CD3Z gene.

375. The composition of claim 374, wherein the TRC subunit gene comprises a TRAC gene.

376. The composition of claim 374, wherein the TRC subunit gene comprises a TRBC gene.

377. The composition of claim 374, wherein the TRC subunit gene comprises a CD3E gene.

378. The composition of claim 374, wherein the TRC subunit gene comprises a CD3D gene.

379. The composition of claim 374, wherein the TRC subunit gene comprises a CD3G gene.

380. The composition of claim 374, wherein the TRC subunit gene comprises a CD3Z gene.

381. The composition of any one of claims 369-380, wherein the transgene comprises a CAR or portion thereof, a cytokine, and/or a reporter gene.

382. The composition of claim 381, wherein the transgene comprises a CAR or portion thereof.

383. A composition comprising a modified human cell comprising:
(a) a first genomic modification comprising a polynucleotide coding for a fusion protein of B2M and HLA-E or HLA-G inserted into a B2M gene, whereby endogenous B2M is partially or completely inactivated and the fusion protein is expressed;
(b) a second genomic modification in a CIITA gene, wherein the CIITA gene is partially or completely inactivated; and
(c) a third genomic modification comprising a first portion of a polynucleotide, wherein the first portion comprises a transgene, inserted into a site with a TRC subunit gene, whereby the TRC subunit gene is partially or completely inactivated and the transgene is expressed.

384. The composition of claim 383, wherein endogenous B2M is completely inactivated.

385. The composition of claim 383 or claim 384, wherein the CIITA gene is completely inactivated.

386. The composition of any one of claims 383-385, wherein the TRC subunit gene is completely inactivated.

387. The composition of any one of claims 383-386, wherein the TRC subunit gene comprises a TRAC, TRBC, CD3E, CD3D, CD3G, or CD3Z gene.

388. The composition of claim 387, wherein the TRC subunit gene comprises a TRAC gene.

389. The composition of claim 387, wherein the TRC subunit gene comprises a TRBC gene.

390. The composition of claim 387, wherein the TRC subunit gene comprises a CD3E gene.

391. The composition of claim 387, wherein the TRC subunit gene comprises a CD3D gene.

392. The composition of claim 387, wherein the TRC subunit gene comprises a CD3G gene.

393. The composition of claim 387, wherein the TRC subunit gene comprises a CD3Z gene.

394. The composition of any one of claims 383-393, wherein the transgene comprises a CAR or portion thereof, a cytokine, and/or a reporter gene.

395. The composition of claim 394, wherein the transgene comprises a CAR or portion thereof.
